(19) United States
(12) Patent Application Publication
(10) Pub. No.: US 2026/0347315 A1
(43) Pub. Date: Nov. 5, 2026

(54) ONIUM SALT, PHOTO-ACID GENERATOR, CHEMICALLY AMPLIFIED RESIST COMPOSITION, AND PATTERNING PROCESS

(71) Applicant: Shin-Etsu Chemical Co., Ltd., Tokyo (JP)

(72) Inventor: Masahiro FUKUSHIMA, Joetsu-shi (JP)

(73) Assignee: Shin-Etsu Chemical Co., Ltd., Tokyo (JP)

(21) Appl. No.: 19/300,823

(22) Filed: Aug. 15, 2025

(30) Foreign Application Priority Data
Aug. 28, 2024 (JP) 2024-146932

(51) Int. Cl. C07C 381/12; G03F 7/004; G03F 7/039; G03F 7/20

(52) U.S. Cl. CPC C07C 381/12; G03F 7/0045; G03F 7/0392; G03F 7/2004

(57) ABSTRACT

The present invention is an onium salt, wherein the onium salt is represented by the following general formula (1). This provides: an onium salt used in a chemically amplified resist composition as a photo-acid generator having excellent solvent solubility, having high sensitivity and high contrast while also suppressing acid diffusion, and having excellent lithography performance in photolithography using high-energy beams; a photo-acid generator consisting of the onium salt; a chemically amplified resist composition containing the photo-acid generator; and a patterning process using the chemically amplified resist composition.

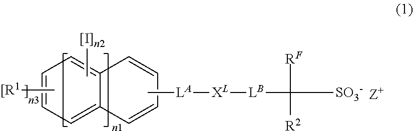

The present invention relates to an onium salt, a photo-acid generator, a chemically amplified resist composition, and a patterning process.

### BACKGROUND ART

In recent years, as the integration and speed of LSIs has increased, there has been a demand for finer pattern rules, and far-ultraviolet lithography and extreme ultraviolet (EUV) lithography are seen as promising next-generation microfabrication technologies.

Photolithography using ArF excimer laser light (ArF lithography) has begun to be partially used in the production of 130 nm node devices, and has become the main lithography technology for 90 nm node devices. Initially, lithography using an F₂ laser with a wavelength of 157 nm was seen as a promising lithography technology for the next node of 45 nm, but delays in development due to various issues have been pointed out. As a result, ArF immersion lithography, for which the numerical aperture (NA) of the projection lens can be designed to be 1.0 or more and a high resolution can be achieved by inserting a liquid with a higher refractive index than air, such as water, ethylene glycol, or glycerin, between the projection lens and the wafer, has suddenly emerged and is now in the practical-application stage. This immersion lithography requires a resist composition that does not easily dissolve in water.

In ArF lithography, in order to prevent deterioration of precise and expensive optical materials, a highly sensitive resist composition that can achieve sufficient resolution with a small amount of exposure is required. The most common method of achieving this is to select components that are highly transparent at a wavelength of 193 nm. For example, with regard to the base polymer, polyacrylic acid and its derivatives, norbornene-maleic anhydride alternating polymers, polynorbornene, ring-opening metathesis polymers, and hydrogenated ring-opening metathesis polymers have been proposed, and some degree of success has been achieved in terms of increasing the transparency of the resin itself.

In recent years, negative tone resists developed with organic solvents have also been attracting attention, along with positive tone resists developed with alkaline aqueous solutions. In order to resolve extremely fine hole patterns that cannot be achieved with positive tone exposure, to resolve with negative tone exposure, a high-resolution positive resist composition is used and development is performed with an organic solvent to form a negative pattern. Furthermore, studies are being conducted to obtain twice the resolution by combining two developments, namely, an alkaline aqueous solution development and an organic solvent development. Conventional positive ArF resist compositions can be used as ArF resist compositions for negative tone development with organic solvents, and patterning processes using these are stated in Patent Documents 1 to 3.

In order to adapt to the rapid miniaturization of recent years, the development of resist compositions is progressing day by day along with the process technology. Various studies have been conducted on photo-acid generators, and sulfonium salts comprising triphenylsulfonium cations and perfluoroalkanesulfonic acid anions are commonly used. However, the acid generated, namely, a perfluoroalkanesulfonic acid, and particularly perfluorooctanesulfonic acid (PFOS), does not decompose easily and is bioaccumulative and toxic, making its application to resist compositions difficult, and currently, photo-acid generators that generate perfluorobutanesulfonic acid are used. However, when this is used in resist compositions, the generated acid diffuses significantly, making it difficult to achieve a high resolution. In response to this problem, various partially fluorinated alkanesulfonic acids and their salts have been developed. For example, Patent Document 1 states, as prior art, photo-acid generators that generate α,α-difluoroalkanesulfonic acid on exposure to light, specifically di(4-tert-butylphenyl) iodonium 1,1-difluoro-2-(1-naphthyl) ethanesulfonate and photo-acid generators that generate α,α,β,β-tetrafluoroalkanesulfonic acid. However, although the fluorine substitution rate of these products is reduced, they do not have decomposable substituents such as ester structures, and are therefore insufficient from the viewpoint of environmental safety due to their ease of decomposition. In addition, there are limitations to the molecular design for changing the size of the alkanesulfonic acid, and the starting material containing fluorine atoms is expensive, among other problems.

In addition, as circuit line widths shrink, the effeCt of contrast degradation due to acid diffusion in resist compositions has been growing more severe. This is because the pattern dimensions are approaching the diffusion length of the acid, leading to a decrease in mask fidelity and deterioration of pattern rectangularity due to an increase in the dimensional deviation on the wafer relative to the value of the dimensional deviation of the mask (mask error factor (MEF)). Therefore, in order to fully harness the benefits of shorter wavelengths and higher NA light sources, it is necessary to increase dissolution contrast or suppress acid diffusion more than with conventional materials. One remedy is to lower the bake temperature, which reduces acid diffusion and improves the MEF, but this inevitably results in lower sensitivity.

Introducing a bulky substituent or polar group into a photo-acid generator is effeCtive for suppressing acid diffusion. Patent Document 4 states a photo-acid generator having 2-acyloxy-1,1,3,3,3-pentafluoropropane-1-sulfonic acid, which has excellent solubility and stability in solvents and enables a wide range of molecular designs. In particular, photo-acid generators having 2-(1-adamantyloxy)-1,1,3,3,3-pentafluoropropane-1-sulfonic acid with a bulky substituent introduced into them have low acid diffusion. Patent Documents 5 to 7 state photo-acid generators into which condensed ring lactones, sultones, or thiolactones have been introduced as polar groups. Although it has been confirmed that the introduction of polar groups results in a certain degree of performance improvement due to the acid diffusion suppression effeCt, it is still insufficient for advanced suppression of acid diffusion, and the lithography performance is not satisfactory from the overall viewpoint of MEF, pattern shape, sensitivity, etc.

Introducing a polar group into the anion of a photo-acid generator is effeCtive for suppressing acid diffusion, but is disadvantageous in terms of solvent solubility. In Patent Documents 8 and 9, attempts have been made to improve solvent solubility by introducing an alicyclic group into the cation moiety of the photo-acid generator, specifically, by introducing a cyclohexane ring or an adamantane ring. Although the introduction of such an alicyclic group improves solubility, a certain number of carbon atoms is required to ensure solubility, and as a result, the molecular structure of the photo-acid generator becomes bulky, which deteriorates lithography performance such as line width roughness (LWR) and dimensional uniformity (CDU) when forming fine patterns.

It has been confirmed that due to iodine atoms having extremely high absorption of EUV light with a wavelength of 13.5 nm, secondary electrons are generated from them during exposure, and this has been attracting attention in EUV lithography. Patent Documents 10 and 11 state photo-acid generators in which an iodine atom has been introduced into the anion, and Patent Document 12 states a polymerizable-group-containing photo-acid generator in which an iodine atom has been introduced into the anion. Patent Document 13 states a photo-acid generator in which an iodine atom has been introduced into both the cation and the anion. Although this has been confirmed to improve lithography performance to a certain extent, iodine atoms are not highly soluble in organic solvents, and there are concerns about precipitation in the solvent.

Patent Document 14 states a photo-acid generator in which a plurality of fluorine atoms have been introduced into the cation. Although the introduction of a plurality of fluorine atoms improves the solvent solubility of the photo-acid generator, this is not sufficient from the viewpoint of EUV absorption, and there is still room for improvement.

Patent Documents 15 to 19 state photo-acid generators and quenchers (acid diffusion suppression agents) in which the cations contain iodine atoms and fluorine atoms. Although it has been confirmed that their performance as resist materials is improved by these developments, they are still not satisfactory in terms of acid diffusion suppression, and there is a demand for the development of resist materials that are useful for forming finer patterns.

### CITATION LIST

### Patent Literature

Patent Document 1: JP2008-281974A
Patent Document 2: JP2008-281975A
Patent Document 3: JP4554665B2
Patent Document 4: JP2007-145797A
Patent Document 5: JP5061484B2
Patent Document 6: JP2016-147879A
Patent Document 7: JP2015-063472A
Patent Document 8: JP5573098B2
Patent Document 9: JP6461919B2
Patent Document 10: JP6720926B2
Patent Document 11: WO2024/057701A1
Patent Document 12: JP6973274B2
Patent Document 13: JP7041204B2
Patent Document 14: JP7389562B2
Patent Document 15: JP2021-123579A
Patent Document 16: JP2021-123580A
Patent Document 17: JP2022-123839A
Patent Document 18: JP2023-088869A
Patent Document 19: JP2023-088870A

### SUMMARY OF INVENTION

### Technical Problem

In response to the recent demand for high-resolution resist patterns, resist compositions using conventional onium salt type photo-acid generators are unable to sufficiently suppress acid diffusion, and this results in deterioration of lithography performance such as contrast, LWR, CDU, MEF, exposure latitude (EL), and depth of focus (DOF). In addition, the occurrence of pattern collapse due to swelling when forming fine patterns is an issue.

The present invention has been created taking into consideration the above circumstances, and aims to provide: an onium salt used in a chemically amplified resist composition as a photo-acid generator having excellent solvent solubility, having high sensitivity and high contrast while also suppressing acid diffusion, and having excellent lithography performance in photolithography using high-energy beams; a photo-acid generator consisting of the onium salt; a chemically amplified resist composition containing the photo-acid generator; and a patterning process using the chemically amplified resist composition.

### Solution to Problem

In order to solve the above problems, the present invention provides an onium salt, wherein the onium salt is represented by the following general formula (1),

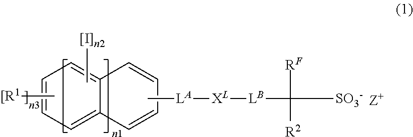

wherein “n1” is 0 or 1; “n2” is an integer from 1 to 4; “n3” is an integer from 0 to 3; provided that when “n1” is 0, 1≤n2+n3≤4, and when “n1” is 1, 1≤n2+n3≤6; R¹ is a halogen atom other than an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” is 2 or 3, each R¹ may be the same as or different from each other, and a plurality of R¹ may be bonded to each other to form a ring together with the carbon atoms to which they are bonded; R² is a hydrogen atom, or a hydrocarbyl group having 1 to 20 carbon atoms and optionally containing a heteroatom; L^{A} and L^{B} are each independently a single bond, an ether bond, an ester bond, a sulfonate ester bond, an amide bond, a sulfonamide bond, a carbonate bond, or a carbamate bond; X^{L} is a single bond, or a hydrocarbylene group having 1 to 40 carbon atoms and optionally containing a heteroatom; R^{F} is a fluorine atom, or a fluorinated saturated hydrocarbyl group having 1 to 6 carbon atoms; and Z⁺ represents an onium cation.

A chemically amplified resist composition containing such an onium salt as a photo-acid generator has excellent solvent solubility, has high sensitivity and high contrast while also suppressing acid diffusion, and has excellent lithography performance in photolithography using high-energy beams.

In addition, in the present invention, the onium salt is preferably represented by the following general formula (1A),

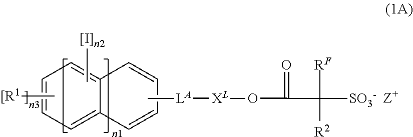

wherein “n1” to “n3”, R¹, R², R^{F}, L^{A}, X^{L}, and Z⁺ are the same as defined above.

For the onium salt of the present invention, ones such as these are more preferred.

In addition, in the present invention, the onium salt represented by the general formula (1A) is preferably represented by the following general formula (1B),

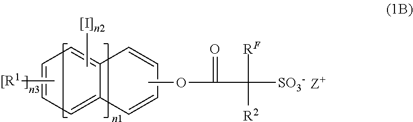

wherein “n1” to “n3”, R¹, R², R^{F}, and Z⁺ are as defined above.

For the onium salt of the present invention, ones such as these are further preferred.

In addition, in the present invention, Z⁺ is preferably a sulfonium cation represented by the following general formula (Z-1) or an iodonium cation represented by the following general formula (Z-2),

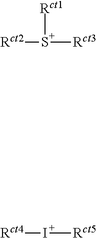

wherein R^{ct1} to R^{ct5} are each independently a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

The onium salt of the present invention preferably has such a cation.

In addition, in the present invention, Z⁺ is preferably a sulfonium cation represented by the following general formula (Z-3),

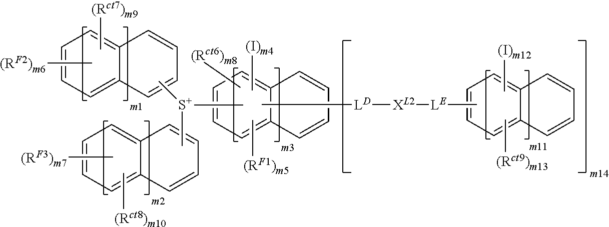

wherein “m1” is 0 or 1; “m2” is 0 or 1; “m3” is 0 or 1; “m4” is an integer from 0 to 4; “m5” is an integer from 0 to 4; “m6” is an integer from 0 to 6; “m7” is an integer from 0 to 6; “m8” is 0, 1 or 2; “m9” is 0, 1 or 2; “m10” is 0, 1 or 2; “m11” is 0 or 1; “m12” is an integer from 0 to 4; “m13” is 0, 1 or 2; “m14” is 0, 1 or 2; provided that when “m1” is 0, 0≤m6+m9≤4, and when “m1” is 1, 0≤m6+m9≤6; when “m2” is 0, 0≤m7+m10≤4, and when “m2” is 1, 0≤m7+m10≤6; when “m3” is 0, 1≤m4+m5+m8+m14≤4, and when “m3” is 1, 1≤m4+m5+m8+m14≤6; when “m11” is 0, 0≤m12+m13≤4, and when “m11” is 1, 0≤m12+m13≤6; also, 1≤m4+m12;
R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms; when “m5” is 2 or more, each R^{F1} may be the same as different from each other, when “m6” is 2 or more, each R^{F2} may be the same as or different from each other, and when “m7” is 2 or more, each R^{F3} may be the same as or different from each other;
R^{ct6} to R^{ct9} are a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “m8” is 2, two R^{ct6} may be the same as or different from each other, and two R^{ct6} may be bonded to each other to form a ring together with the carbon atoms to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two R^{ct9} may be bonded to each other to form a ring together with the carbon atoms to which they are bonded;
also, the aromatic rings directly bonded to S⁺ in the sulfonium cation may be bonded to each other to form a ring together with S⁺;
L^{D} and L^{E} are each independently a single bond, an ether bond, an ester bond, an amide bond, a sulfonate ester bond, a sulfonamide bond, a carbonate bond, or a carbamate bond; and
X^{L2} is a single bond, or a hydrocarbylene group having 1 to 40 carbon atoms and optionally containing a heteroatom.

The onium salt of the present invention also preferably has such a cation.

In addition, the present invention provides a photo-acid generator, wherein the photo-acid generator contains the onium salt described above.

Such a photo-acid generator can be used as a photoacid generator contained in a chemically amplified resist composition that has excellent solvent solubility, will have high sensitivity and high contrast while also suppressing acid diffusion, and will have excellent lithography performance in photolithography using high-energy beams.

In addition, the present invention provides a chemically amplified resist composition, wherein the chemically amplified resist composition contains the photo-acid generator described above.

Such a chemically amplified resist composition will have excellent solvent solubility, will have high sensitivity and high contrast while also suppressing acid diffusion, and will have excellent lithography performance in photolithography using high-energy beams.

In addition, in the present invention, the chemically amplified resist composition preferably further contains a base polymer containing at least one repeating unit selected from the group consisting of a repeating unit represented by the following general formula (a1), a repeating unit represented by the following general formula (a2), and a repeating unit represented by the following general formula (a3),

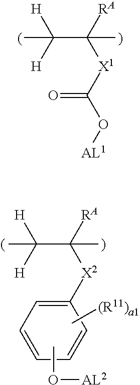

wherein “a1” is an integer from 0 to 4;
R^{A} is each independently a hydrogen atom, a fluorine atom, a methyl group, or a trifluoromethyl group;
X¹ is a single bond, a phenylene group, a naphthylene group, *—C(═O)—OX¹¹—, or *—C(═O)—NH—X¹¹, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ is a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² is a single bond, *—C(═O)—O—, or *—C(═O)—NH—;
“*” represents a bond to a main chain carbon atom;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2, 3, or 4, each R¹¹ may be the same as or different from each other; and
A^{L1} and A^{L2} are each independently an acid-labile group,

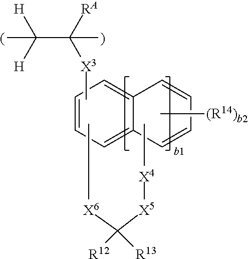

wherein “b1” is 0 or 1; “b2” is an integer from 0 to 3 when “b1” is 0, and an integer from 0 to 5 when “b1” is 1;
R^{A} is a hydrogen atom, a fluorine atom, a methyl group, or a trifluoromethyl group;
X³ is a single bond, *—C(═O)—O—, or *—C(═O)—NH—; “*” represents a bond to a main chain carbon atom;
X⁴ is a single bond, an aliphatic hydrocarbylene group having 1 to 4 carbon atoms, a carbonyl group, a sulfonyl group, or a group obtained by combining these;
X⁵ and X⁶ are each independently an oxygen atom or a sulfur atom; provided that X⁴ and X⁶ are bonded to adjacent carbon atoms of an aromatic ring;
R¹² and R¹³ are each independently a hydrogen atom or a hydrocarbyl group having 1 to 20 carbon atoms and optionally containing a heteroatom; also, R¹² and R¹³ may be bonded to each other to form a ring together with the carbon atom to which they are bonded;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, or a hydrocarbylthio group having 1 to 20 carbon atoms and optionally containing a heteroatom, or —N(R^{14A})(R^{14B}); R^{14A} and R^{14B} are each independently a hydrogen atom or a hydrocarbyl group having 1 to 6 carbon atoms; and when “b2” is 2 or more, each R¹⁴ may be the same as or different from each other, and a plurality of R¹⁴ may be bonded to each other to form a ring together with the carbon atoms of the aromatic ring to which they are bonded.

The chemically amplified resist composition of the present invention preferably comprises such a base polymer.

In addition, the base polymer preferably further contains at least one repeating unit selected from the group consisting of a repeating unit represented by the following general formula (b1) and a repeating unit represented by the following general formula (b2),

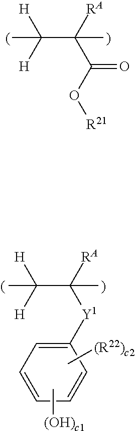

wherein “c1” is an integer from 1 to 4; “c2” is an integer from 0 to 4; and 1≤c1+c2≤5;
R^{A} is each independently a hydrogen atom, a fluorine atom, a methyl group, or a trifluoromethyl group;
Y¹ is a single bond or *—C(═O)—O—; “*” represents a bond to a main chain carbon atom;
R²¹ is a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R²² is a halogen atom, a hydroxy group, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” is 2, 3, or 4, each R²² may be the same as or different from each other.

In the present invention, the base polymer preferably has such a repeating unit.

In addition, the base polymer preferably further contains at least one constituent selected from a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5),

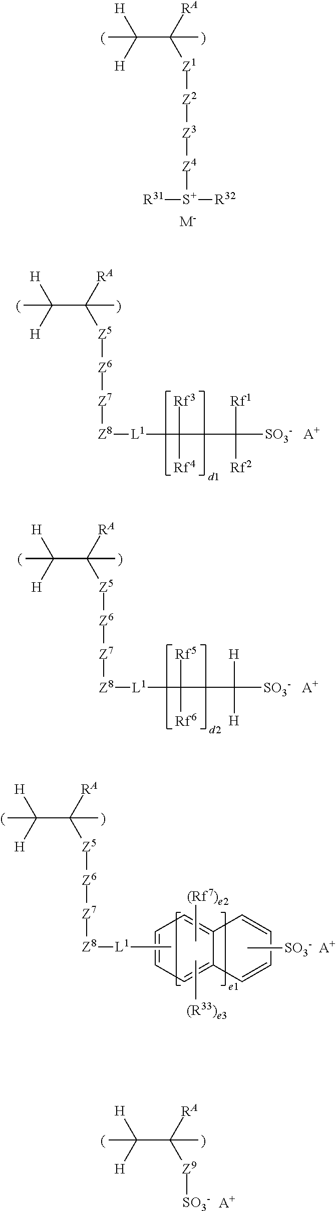

wherein “dl” and “d2” each independently represent an integer from 0 to 3; “e1” is 0 or 1; “e2” is an integer from 0 to 4; “e3” is an integer from 0 to 4; provided that when “e1” is 0, 0≤e2+e3≤4, and when “e1” is 1, 0≤e2+e3≤6;
R^{A} is each independently a hydrogen atom, a fluorine atom, a methyl group, or a trifluoromethyl group;
Z¹ is a single bond, or a phenylene group optionally containing a substituent;
Z² is a single bond, **—C(═O)—O—Z²¹—, **—C(═O)—NH—Z²¹—, or **—O—Z²¹—; Z²¹ is an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ is a single bond, an ether bond, an ester bond, an amide bond, a sulfonate ester bond, a sulfonamide bond, a carbonate bond, or a carbamate bond;
Z⁴ is a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ is each independently a single bond, a phenylene group or a naphthylene group optionally containing a substituent, or *—C(═O)—O—Z⁵¹—; Z⁵¹ is an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ is a single bond, an ether bond, an ester bond, an amide bond, a sulfonate ester bond, a sulfonamide bond, a carbonate bond, or a carbamate bond;
Z⁷ is each independently a single bond, ***—Z⁷¹—C(═O)—O—, ***—C(═O)—NH—Z⁷¹—, or ***—O—Z⁷¹—; Z⁷¹ is a hydrocarbylene group having 1 to 20 carbon atoms and optionally containing a heteroatom;
Z⁸ is each independently a single bond, ****—Z⁸¹—C(═O)—O—, ****—C(═O)—NH—Z⁸¹— or ****—O—Z⁸¹—; Z⁸¹ is a hydrocarbylene group having 1 to 20 carbon atoms and optionally containing a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹— or *—O—Z⁹¹—; Z⁹¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
“*” represents a bond to a main chain carbon atom; “**” represents a bond to Z¹; “***” represents a bond to Z⁶; “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom, or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms; provided that all Rf⁵ and Rf⁶ are not simultaneously hydrogen atoms;
Rf⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R³¹ and R³² are each independently a hydrocarbyl group having 1 to 20 carbon atoms and optionally containing a heteroatom; also, R³¹ and R³² may be bonded to each other to form a ring together with the sulfur atom to which they are bonded;
R³³ is a halogen atom other than a fluorine atom, or hydrocarbyl group having 1 to 20 carbon atoms and optionally containing a heteroatom; when “e3” is 2, 3, or 4, each R³³ may be the same as or different from each other, and a plurality of R³³ may be bonded to each other to form a ring together with the carbon atoms to which they are bonded;
M⁻ is a non-nucleophilic counter ion; and
A⁺ is an onium cation.

In the present invention, the base polymer preferably has such a repeating unit.

In addition, the chemically amplified resist composition preferably further contains at least one constituent selected from the group consisting of an organic solvent, a quencher, a photo-acid generator other than the photo-acid generator, and a surfactant.

The chemically amplified resist composition of the present invention may contain these additives.

In addition, in the present invention, a patterning process is provided, wherein the patterning process contains a step of forming a resist film on a substrate using the chemically amplified resist composition described above, a step of exposing the resist film with a high-energy beam, and a step of developing the exposed resist film using a developer.

Such a patterning process can provide a patterning process using a chemically amplified resist composition that has excellent solvent solubility, and has high sensitivity, high contrast, and excellent lithography performance while also suppressing acid diffusion in photolithography using high-energy beams.

When doing this, the high-energy beam is preferably KrF excimer laser light, ArF excimer laser light, an electron beam, or extreme ultraviolet light having a wavelength of 3 to 15 nm as the high-energy beam.

These can be used as the high-energy beam.

### Advantageous EffeCts of Invention

As described above, when patterning is performed using a chemically amplified resist composition containing the onium salt of the present invention as a photo-acid generator, particularly in photolithography using high-energy beams such as KrF excimer laser light, ArF excimer laser light, an electron beam (EB), EUV, etc., it is possible to form a resist pattern that has excellent solvent solubility, has high sensitivity and high contrast while also suppressing acid diffusion, has excellent lithography performance in terms of LWR, CDU, MEF, EL, DOF, etc., and suppresses the collapse of the resist pattern.

### DESCRIPTION OF EMBODIMENTS

As described above, there has been a demand for the development of an onium salt used in a chemically amplified resist composition as a photo-acid generator that has excellent solvent solubility, has high sensitivity and high contrast while also suppressing acid diffusion, and has excellent lithography performance in photolithography using high-energy beams, a photo-acid generator consisting of the onium salt, a chemically amplified resist composition that contains the photo-acid generator, and a patterning process that uses the chemically amplified resist composition.

As a result of intensive study conducted by the inventor to achieve the above object, he discovered that onium salts containing a monofluoroalkanesulfonate anion having an aromatic ring containing an iodine atom have excellent solvent solubility, and that chemically amplified resist compositions using these as a photo-acid generator have high sensitivity and high contrast while also suppressing acid diffusion, have excellent lithography performance in terms of LWR, CDU, MEF, EL, DOF, etc., and are extremely effeCtive for suppressing the collapse of the pattern in forming fine patterns. This leads him to create the present invention.

That is, the present invention is an onium salt, wherein the onium salt is represented by the following general formula (1),

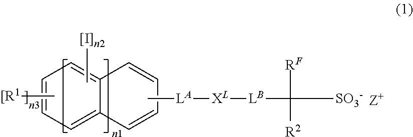

wherein “n1” is 0 or 1; “n2” is an integer from 1 to 4; “n3” is an integer from 0 to 3; provided that when “n1” is 0, 1≤n2+n3≤4, and when “n1” is 1, 1≤n2+n3≤6; R¹ is a halogen atom other than an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” is 2 or 3, each R¹ may be the same as or different from each other, and a plurality of R¹ may be bonded to each other to form a ring together with the carbon atoms to which they are bonded; R² is a hydrogen atom, or a hydrocarbyl group having 1 to 20 carbon atoms and optionally containing a heteroatom; L^{A} and L^{B} are each independently a single bond, an ether bond, an ester bond, a sulfonate ester bond, an amide bond, a sulfonamide bond, a carbonate bond, or a carbamate bond; X^{L} is a single bond, or a hydrocarbylene group having 1 to 40 carbon atoms and optionally containing a heteroatom; R^{F} is a fluorine atom, or a fluorinated saturated hydrocarbyl group having 1 to 6 carbon atoms; and Z⁺ represents an onium cation.

Hereinafter, the present invention will be described in detail. However, the present invention is not limited thereto.

### [Onium Salt]

The onium salt of the present invention is represented by the following general formula (1).

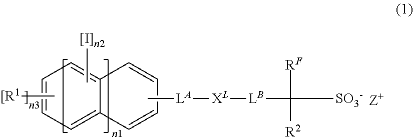

In the formula, “n1” is 0 or 1; “n2” is an integer from 1 to 4; “n3” is an integer from 0 to 3; provided that when “n1” is 0, 1≤n2+n3≤4, and when “n1” is 1, 1≤n2+n3≤6; R¹ is a halogen atom other than an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” is 2 or 3, each R¹ may be the same as or different from each other, and a plurality of R¹ may be bonded to each other to form a ring together with the carbon atoms to which they are bonded; R² is a hydrogen atom, or a hydrocarbyl group having 1 to 20 carbon atoms and optionally containing a heteroatom; L^{A} and L^{B} are each independently a single bond, an ether bond, an ester bond, a sulfonate ester bond, an amide bond, a sulfonamide bond, a carbonate bond, or a carbamate bond; X^{L} is a single bond, or a hydrocarbylene group having 1 to 40 carbon atoms and optionally containing a heteroatom; RE is a fluorine atom, or a fluorinated saturated hydrocarbyl group having 1 to 6 carbon atoms; and Z⁺ represents an onium cation.

In the general formula (1), “n1” is 0 or 1. When “n1” is 0, the structure is a benzene ring, and when “n1” is 1, it is a naphthalene ring; however, from the viewpoint of solvent solubility, “n1” is preferably 0, giving a benzene ring. “n2” is an integer from 1 to 4. From the viewpoint of raw material procurement, “n2” is preferably 1 to 3. “n3” is an integer from 0 to 3. From the viewpoint of raw material procurement, “n3” is preferably 0 or 1. Provided that when “n1” is 0, 1≤n2+n3≤4, and when “n1” is 1, 1≤n2+n3≤6.

In the general formula (1), R¹ is a halogen atom other than a iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Examples of the halogen atom include a fluorine atom, a chlorine atom, a bromine atom, etc., but it is preferably a fluorine atom. The hydrocarbyl group may be saturated or unsaturated, and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icocyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group, and a naphthyl group; aralkyl groups having 7 to 20 carbon atoms, such as a benzyl group, a 1-phenylethyl group, and a 2-phenylethyl group; and groups obtained by combining these. Of these, aryl groups are preferred. In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. When “n3” is 2 or 3, two R¹ may be the same as or different from each other. In addition, when “n3” is 2 or 3, two R¹ may be bonded to each other to form a ring together with the carbon atoms to which they are bonded. The ring is preferably a 5- to 8-member ring.

In the general formula (1), R² is a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include the same ones as for R¹, but are not limited thereto. From the viewpoint of raw material procurement, R² is preferably a hydrogen atom.

In the general formula (1), L^{A} and L^{B} are each independently a single bond, an ether bond, an ester bond, a sulfonate ester bond, an amide bond, a sulfonamide bond, a carbonate bond, or a carbamate bond. Among these, a single bond, an ether bond, an ester bond, or a sulfonate ester bond is preferable, and an ether bond or an ester bond is more preferable.

In the general formula (1), X^{L} is a single bond, or a hydrocarbylene group having 1 to 40 carbon atoms and optionally containing a heteroatom. The hydrocarbylene group may be linear, branched, or cyclic, and specific examples thereof include an alkanediyl group and a divalent saturated cyclic hydrocarbon group. Examples of the heteroatom include an oxygen atom, a nitrogen atom, and a sulfur atom.

Specific examples of the hydrocarbylene group having 1 to 40 carbon atoms optionally containing a heteroatom represented by X^{L} include, but are not limited to, those shown below. In addition, in the following formulae, “*” represents the bond to L^{A} and L^{B}.

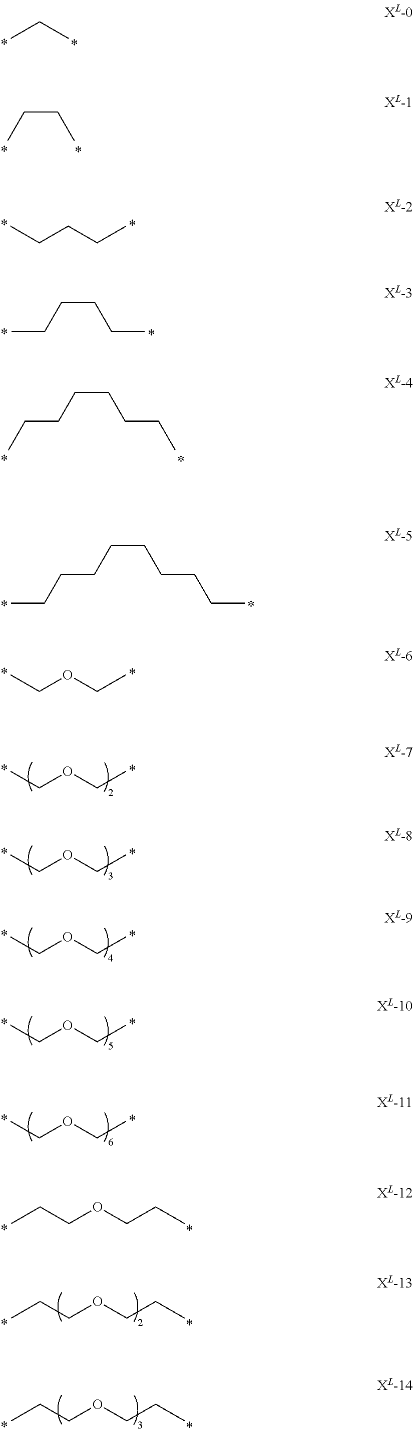

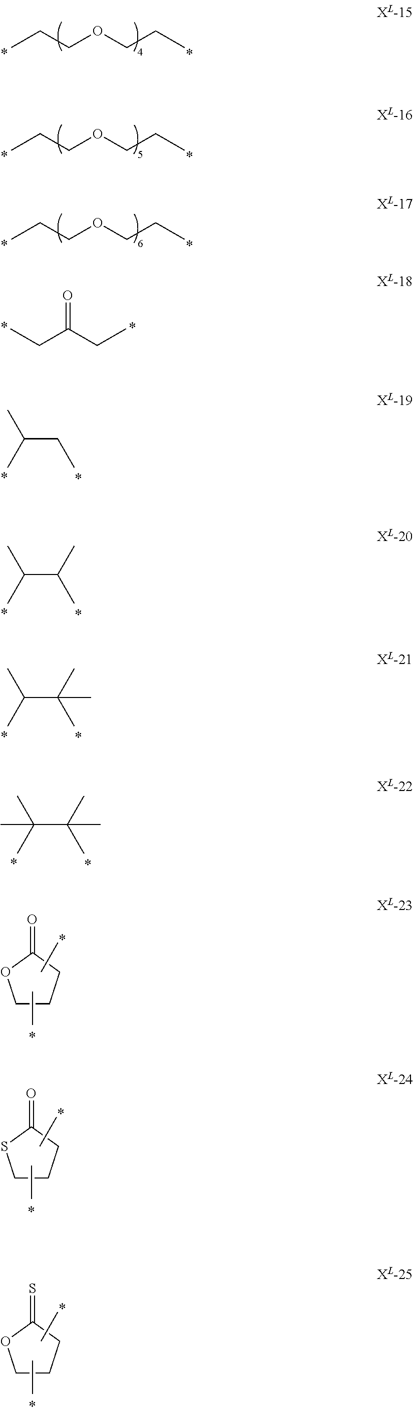

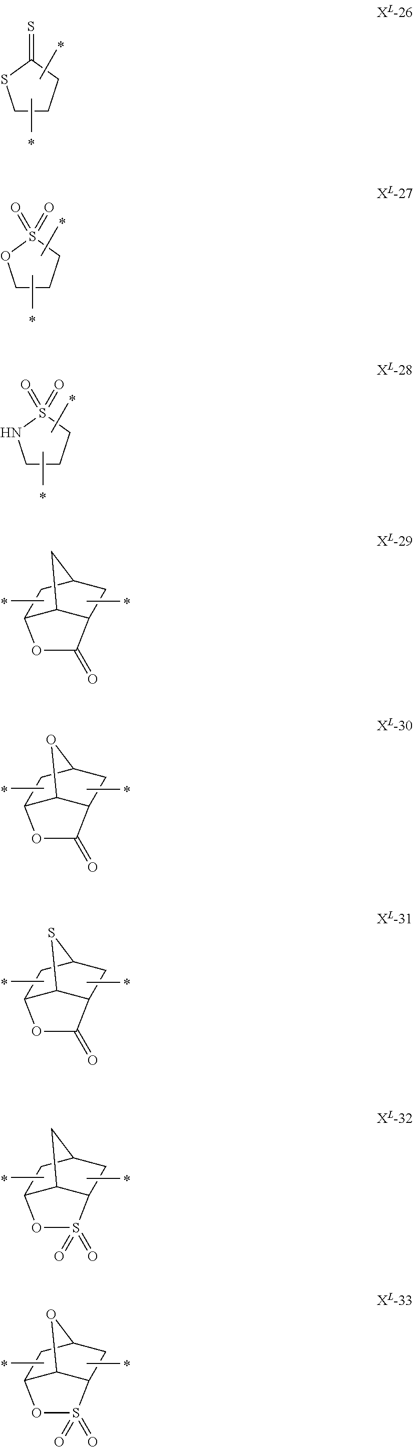

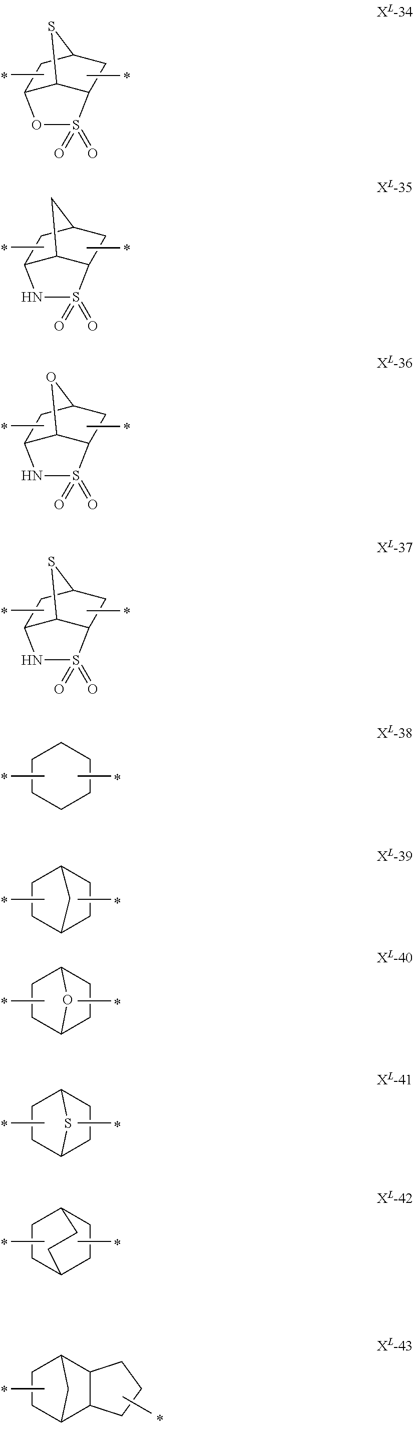

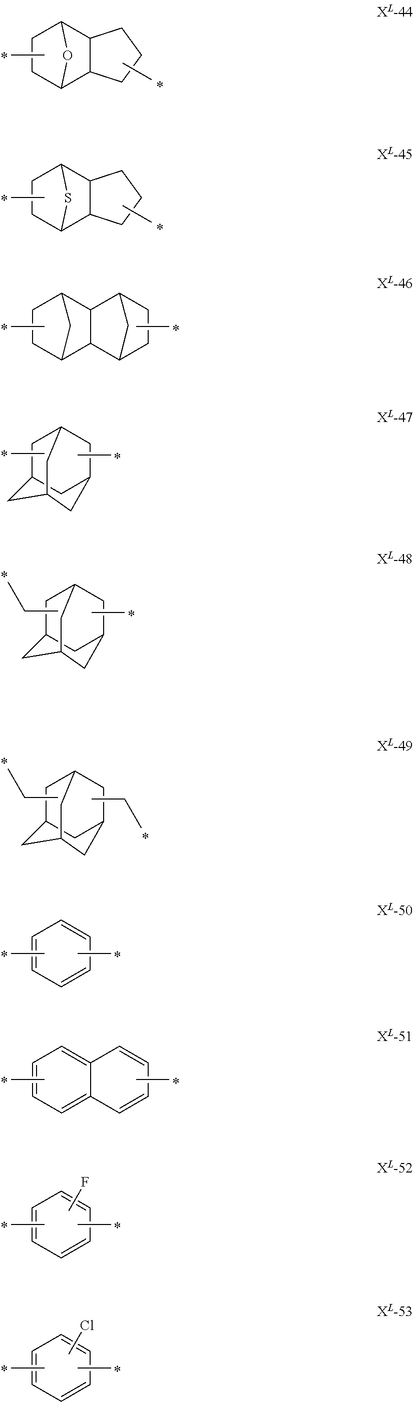

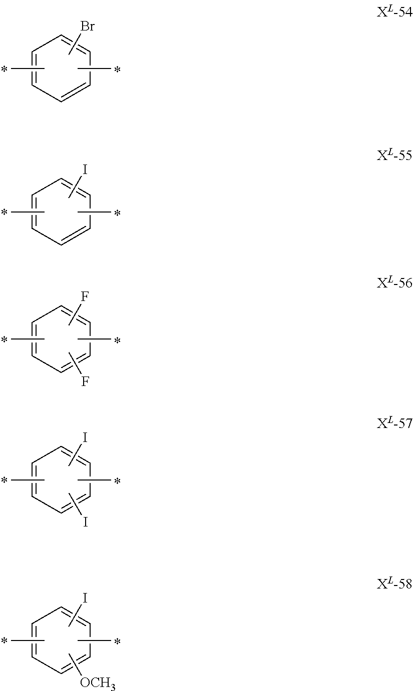

Of these, XL-0 to XL-22, XL-29 to XL-34, and XL-47 to XL-58 are preferable.

In the general formula (1), R^{F} is a fluorine atom or a fluorinated saturated hydrocarbyl group having 1 to 6 carbon atoms. The fluorinated saturated hydrocarbyl group having 1 to 6 carbon atoms is preferably a trifluoromethyl group.

The onium salt is preferably one represented by the following general formula (1A).

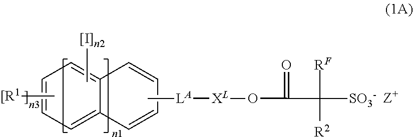

In the formula, “n1” to “n3”, R¹, R², R^{F}, L^{A}, X^{L}, and Z⁺ are as defined above.

The onium salt represented by the general formula (1A) is preferably one represented by the following general formula (1B).

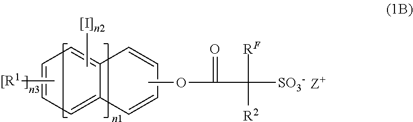

In the formula, “n1” to “n3”, R¹, R², R^{F}, and Z⁺ are the same as defined above.

Examples of the anion of the onium salt represented by the general formula (1) include, but are not limited to, those shown below. In addition, the positions of the substituents on the aromatic ring are not limited thereto. In addition, in the following formulae, Me is a methyl group.

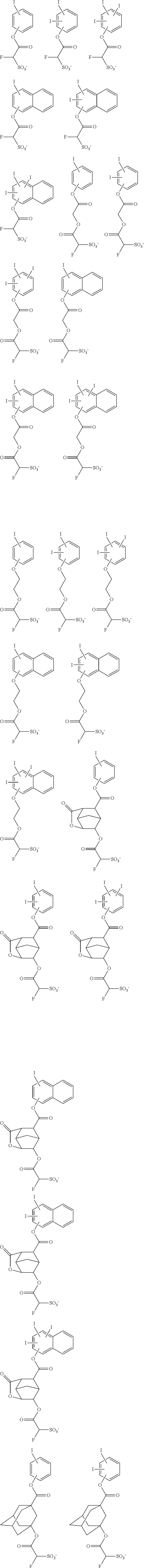

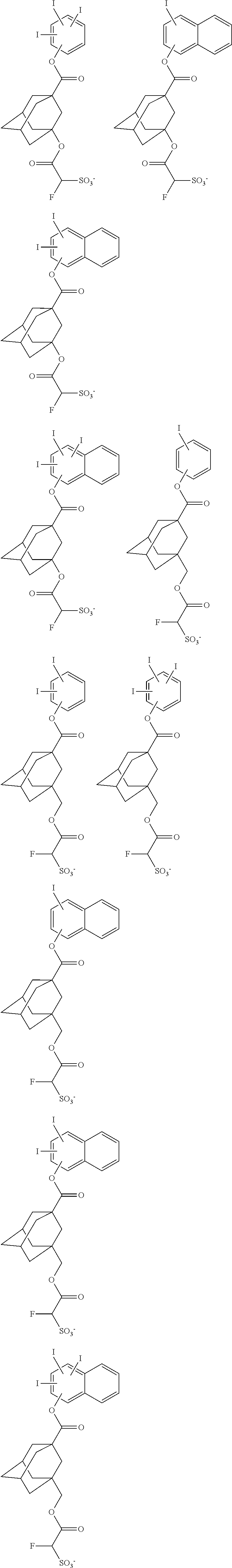

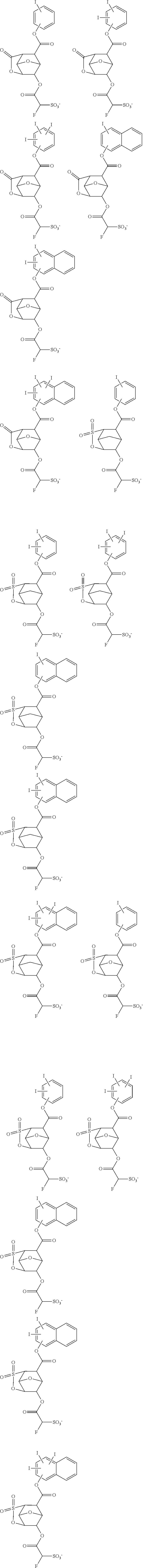

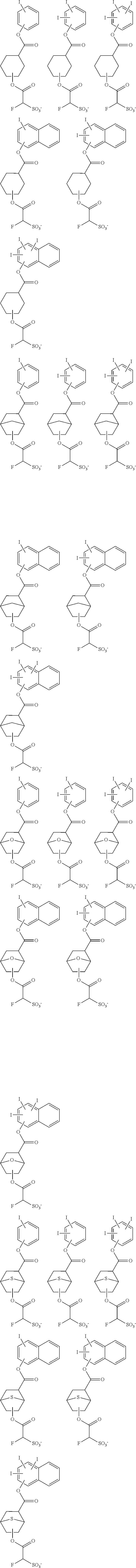

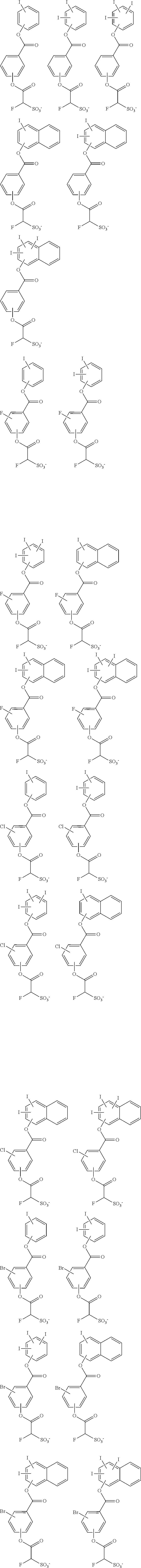

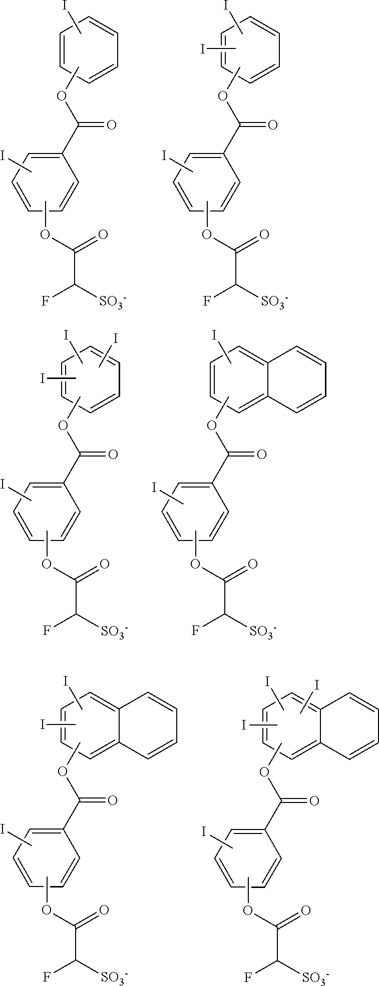

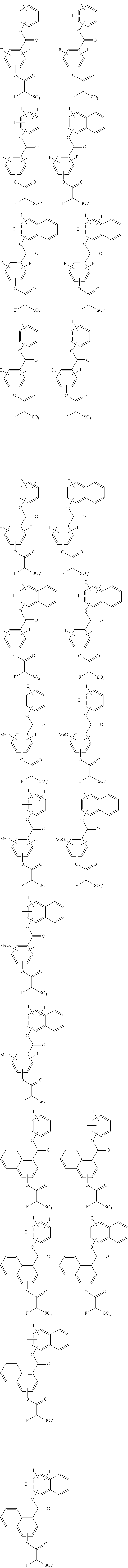

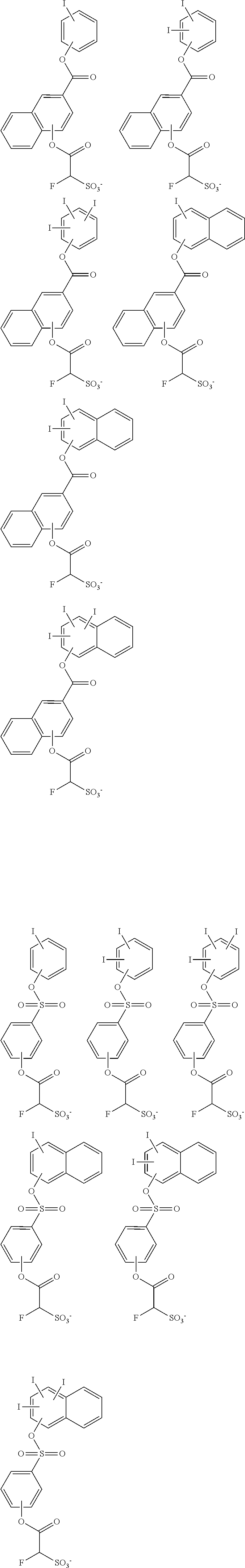

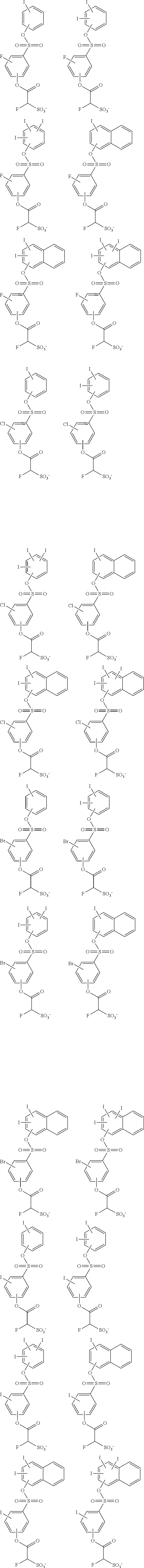

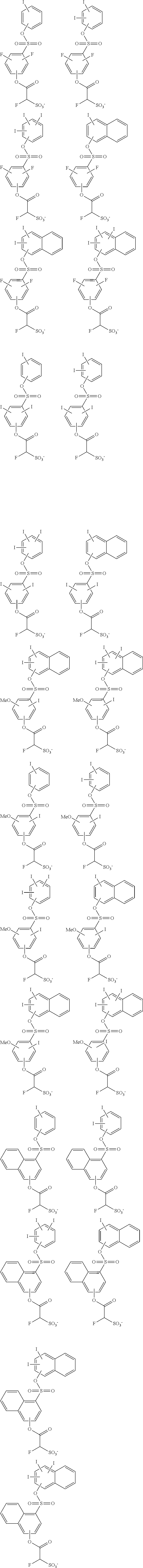

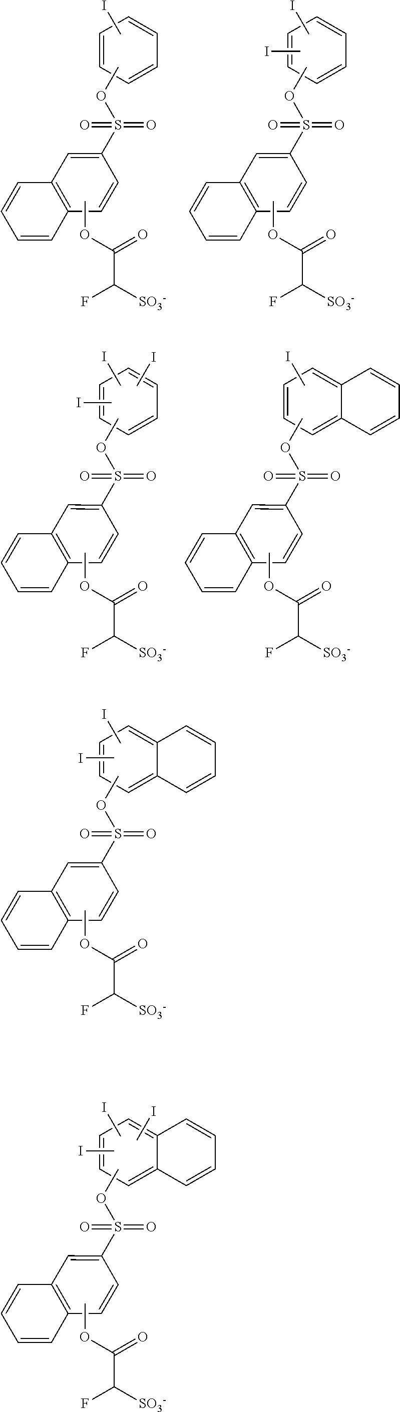

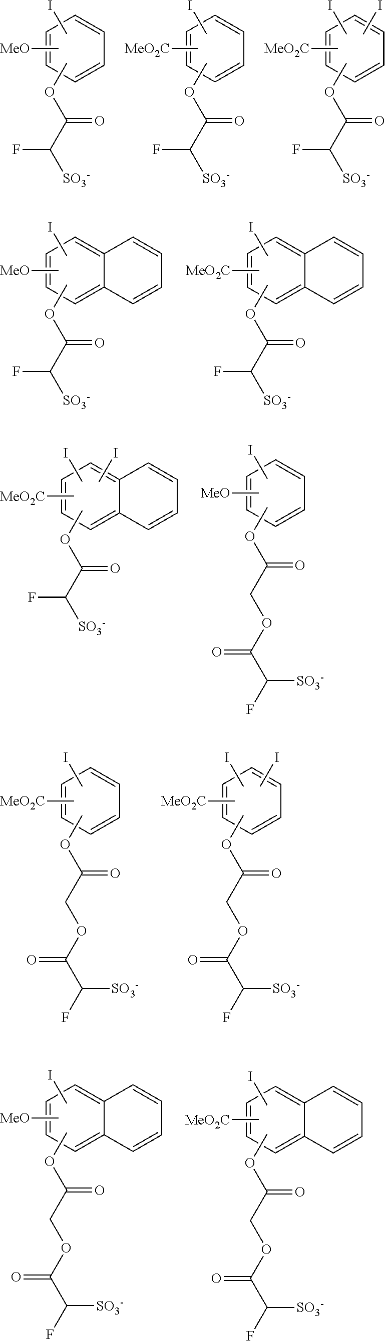

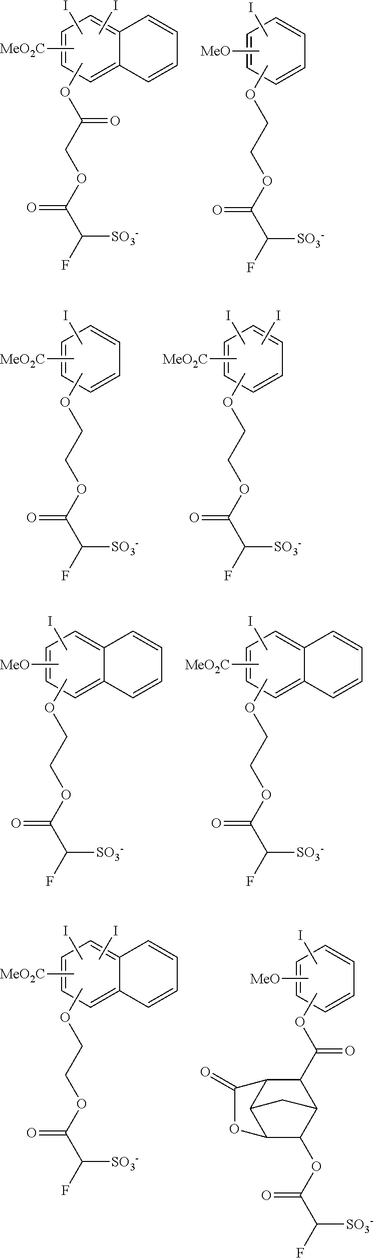

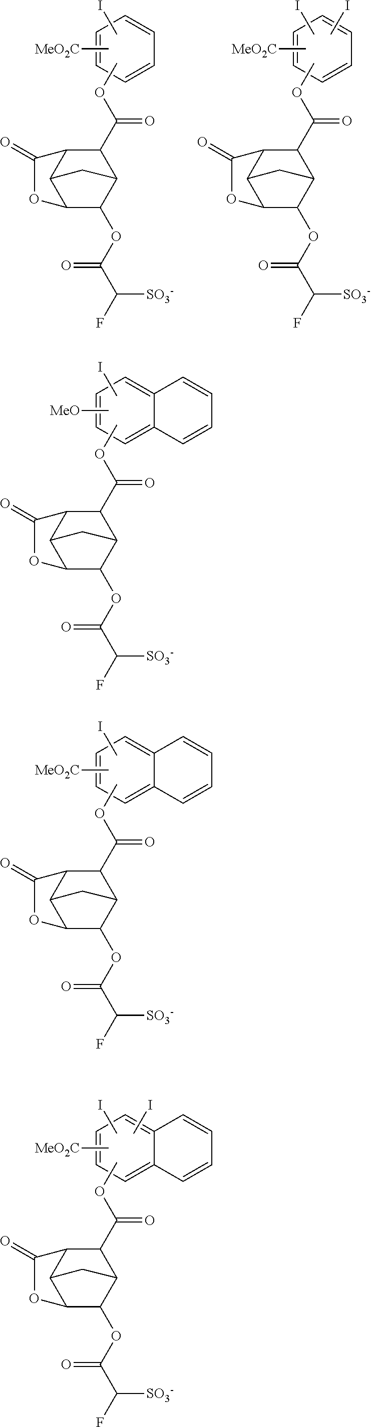

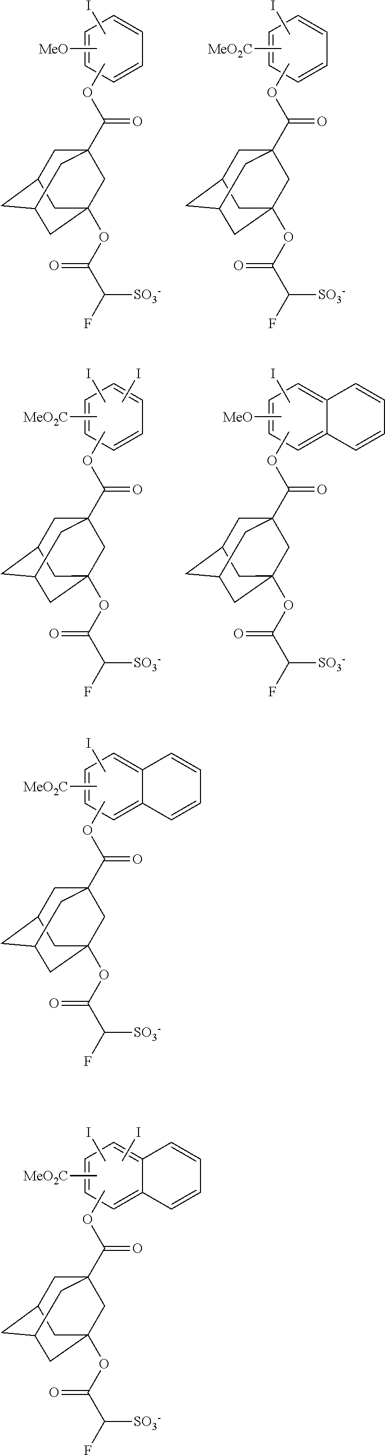

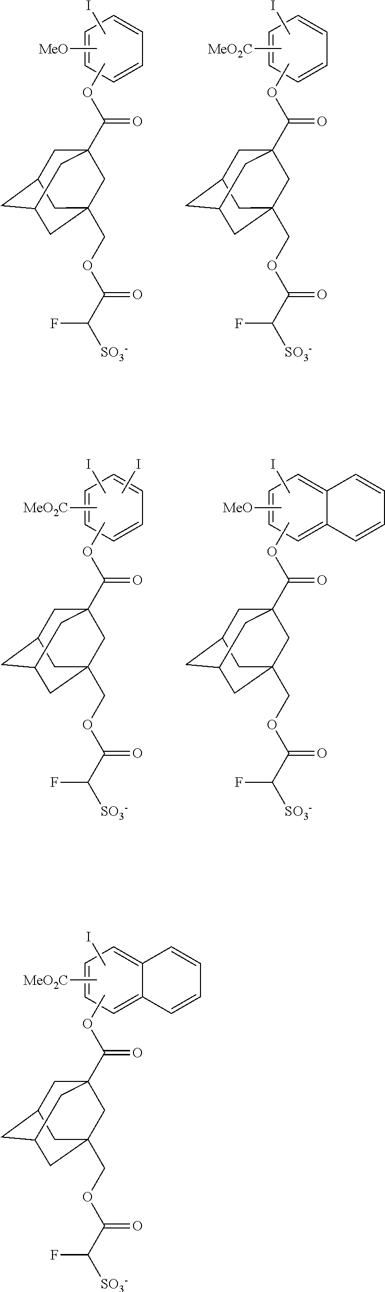

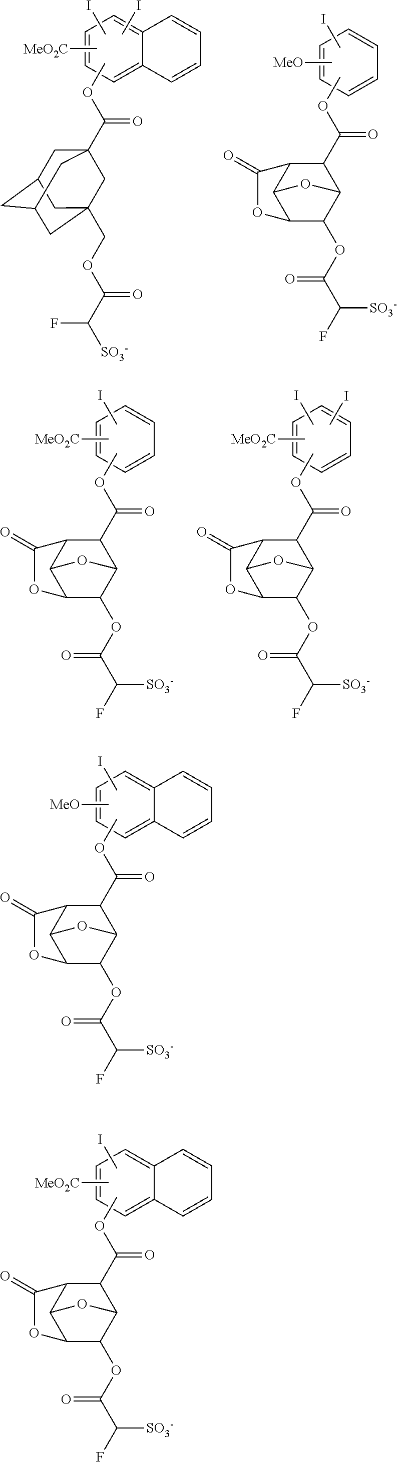

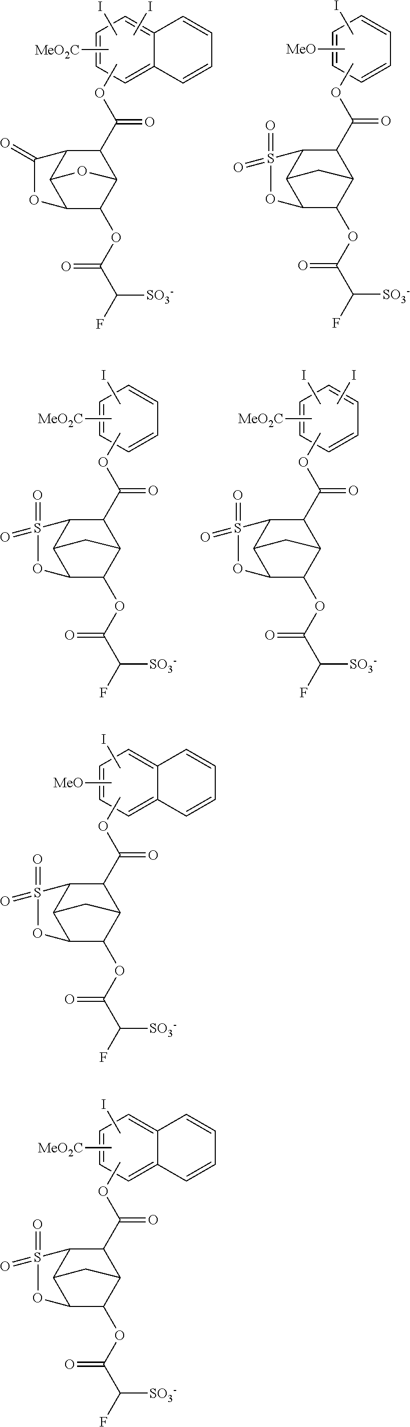

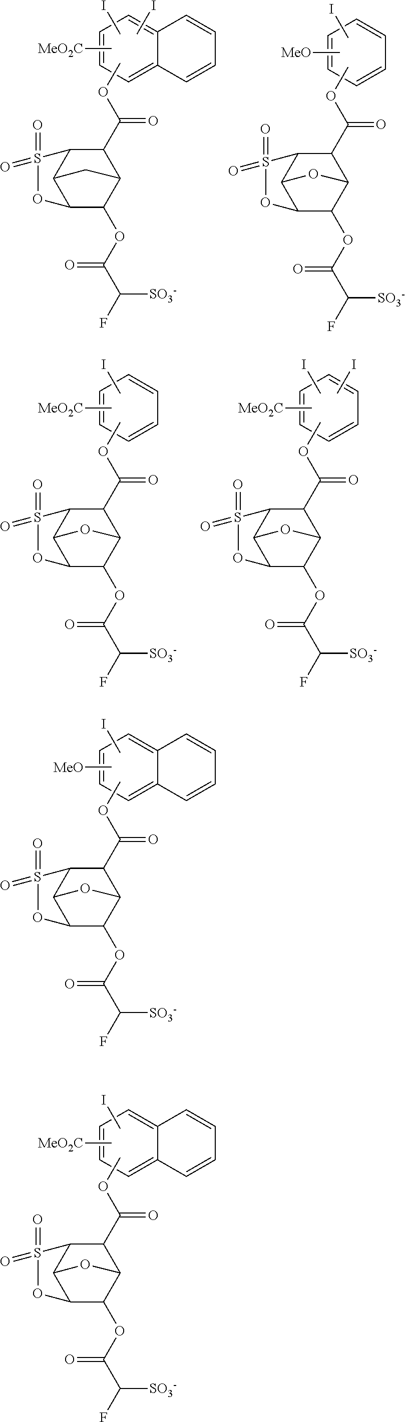

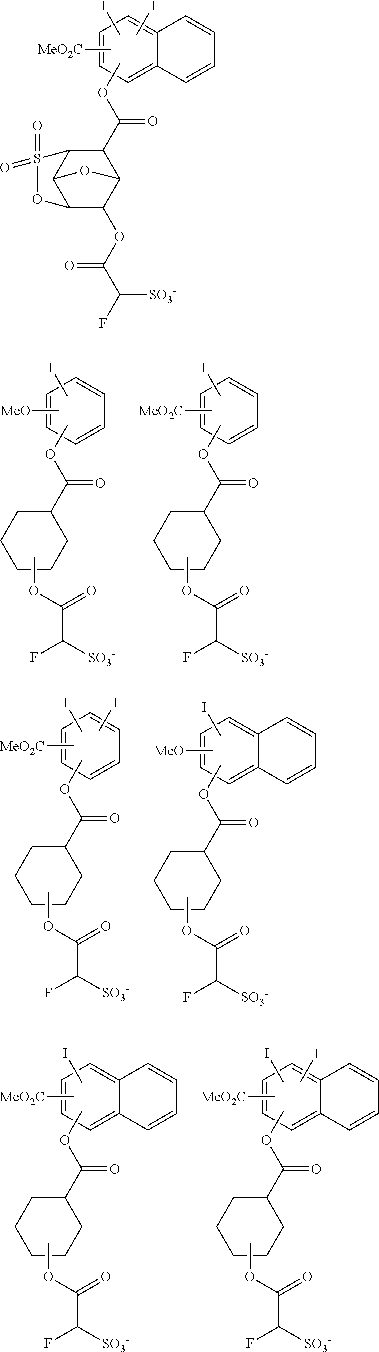

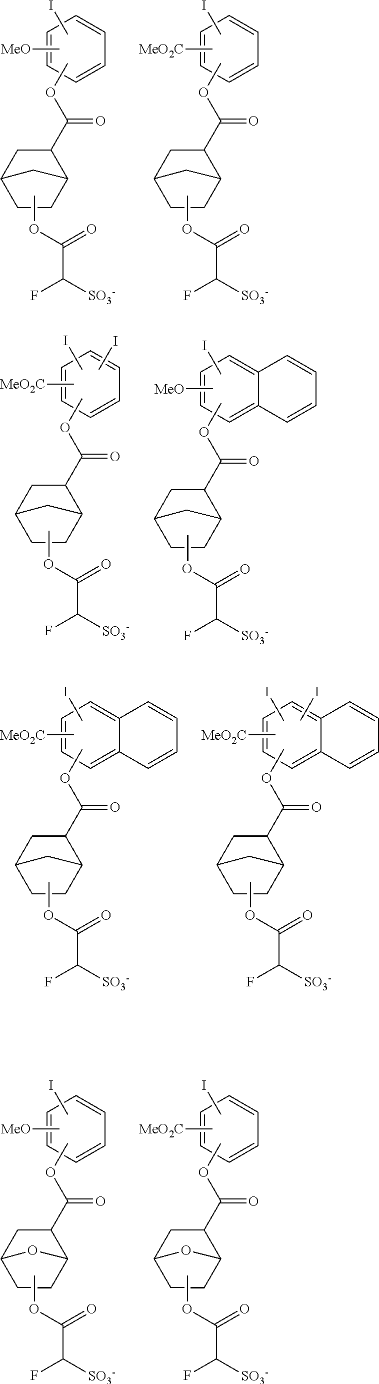

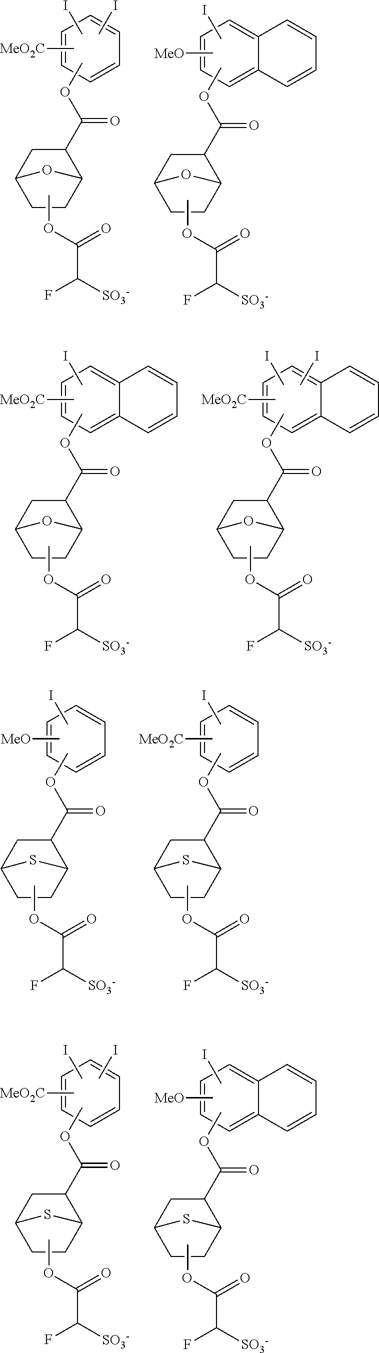

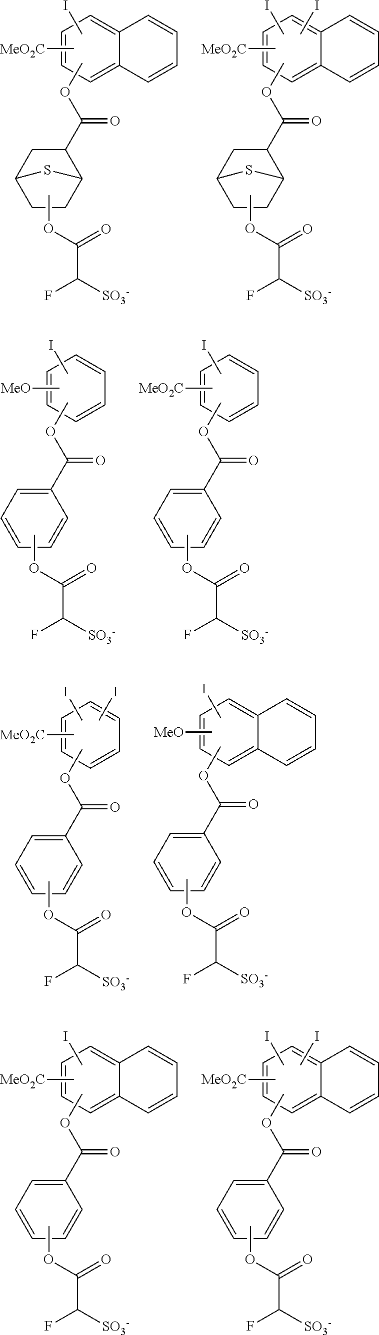

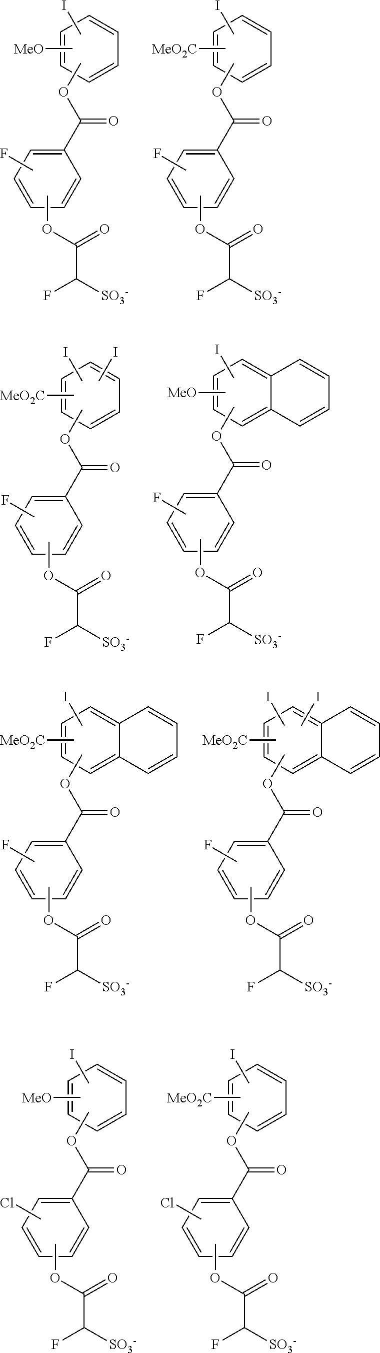

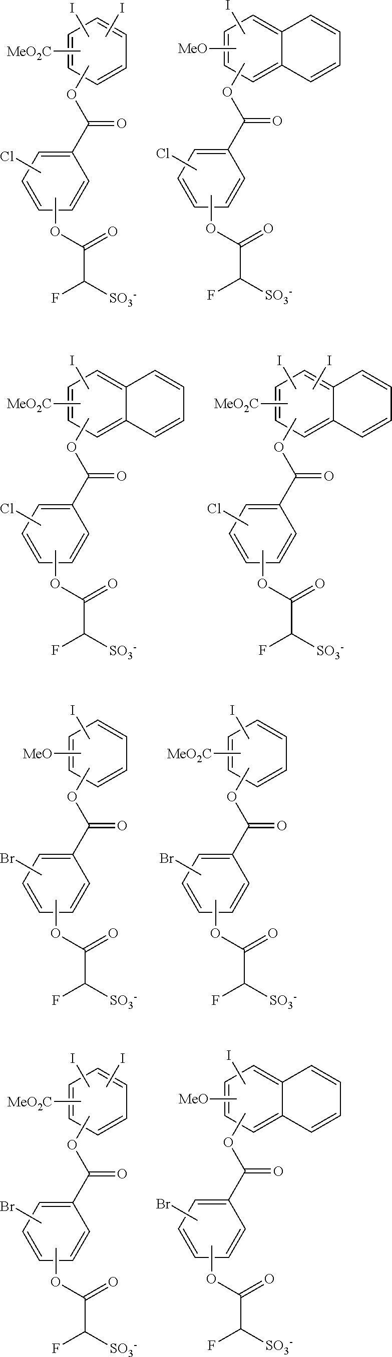

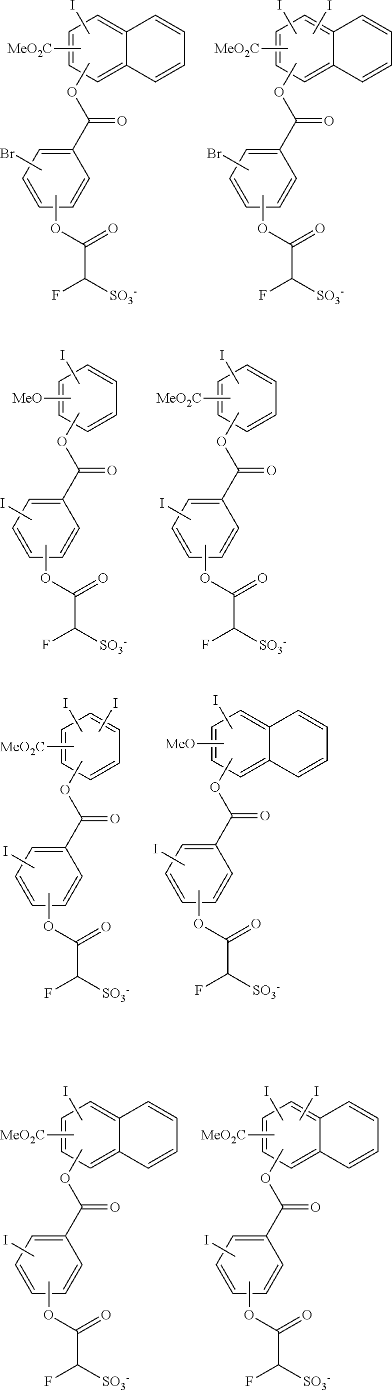

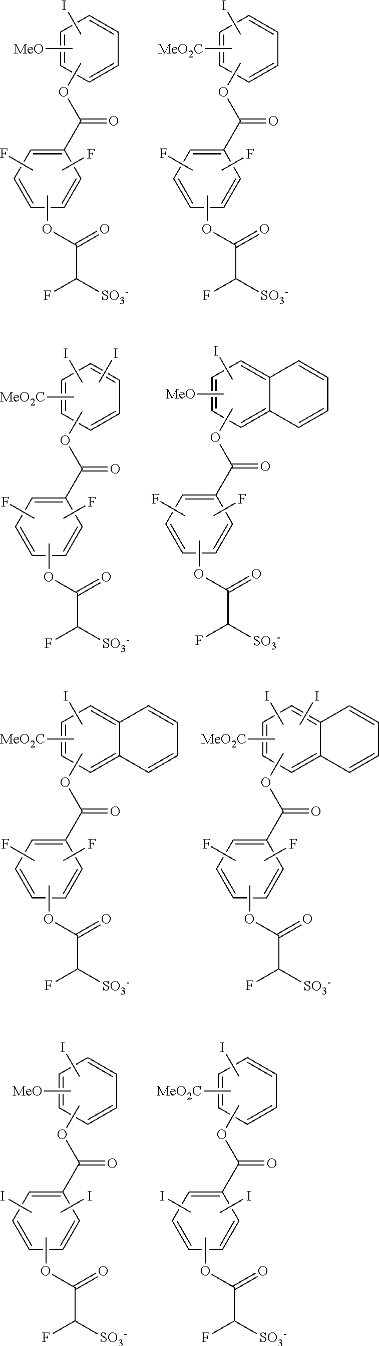

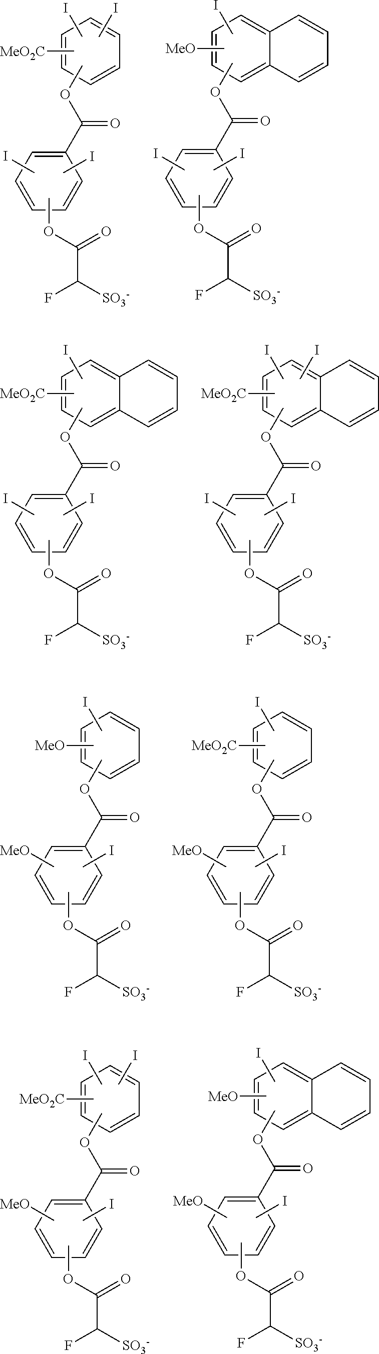

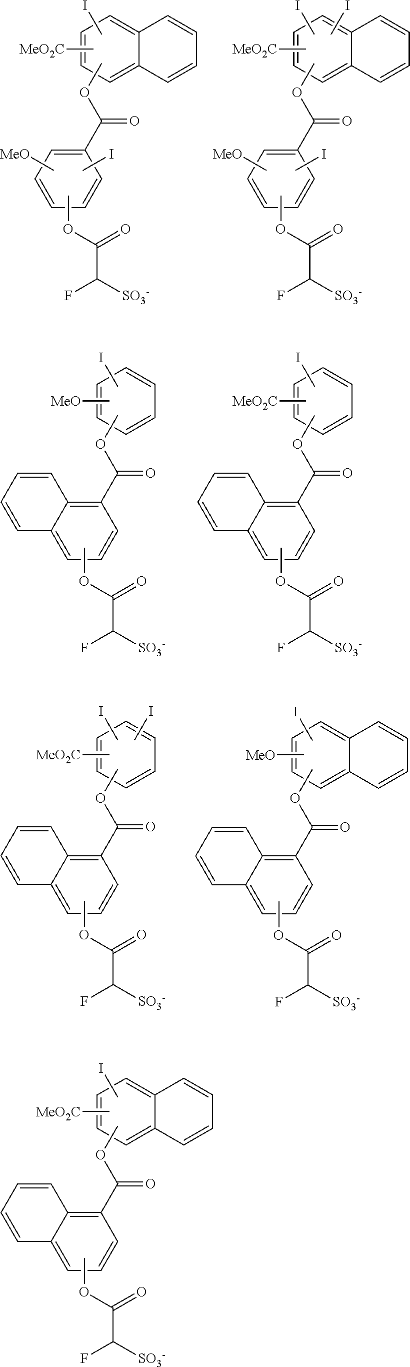

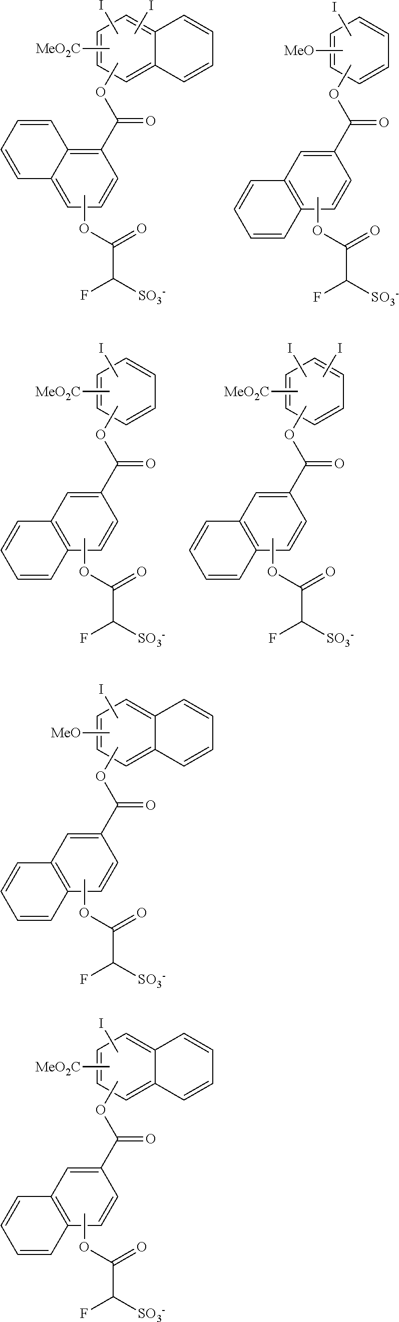

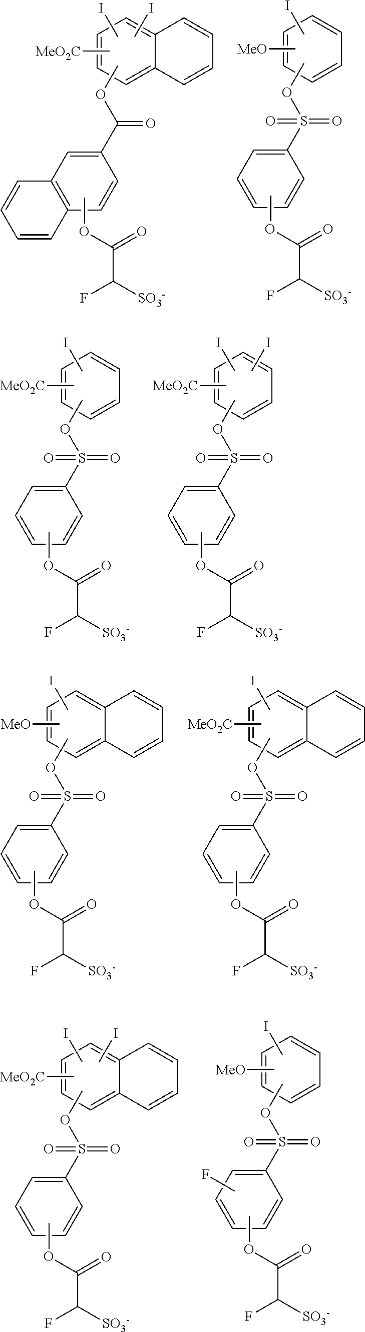

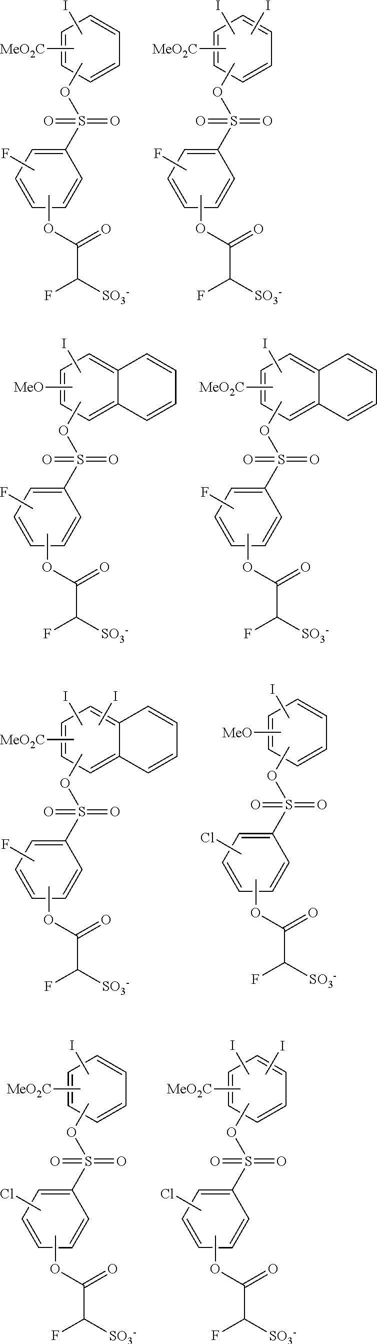

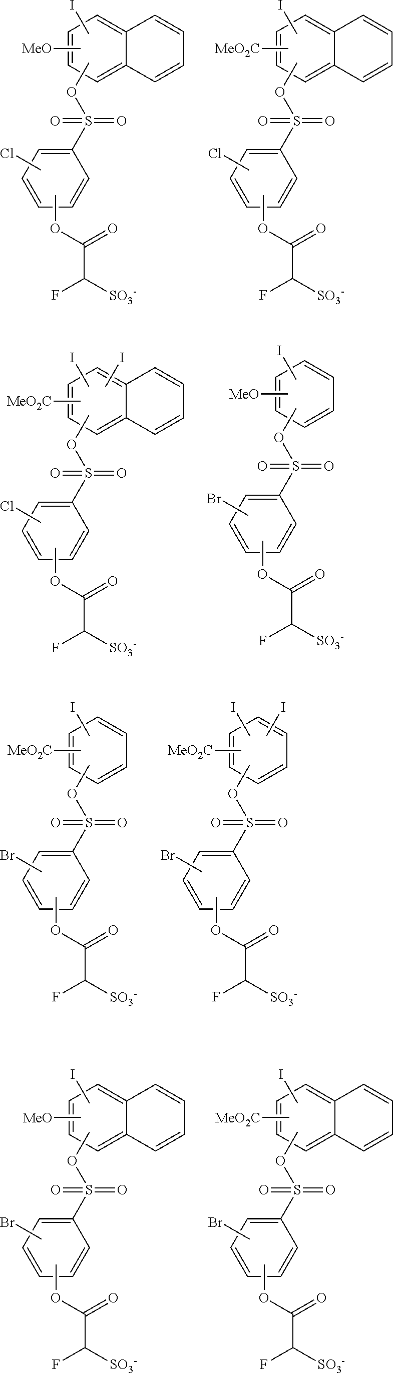

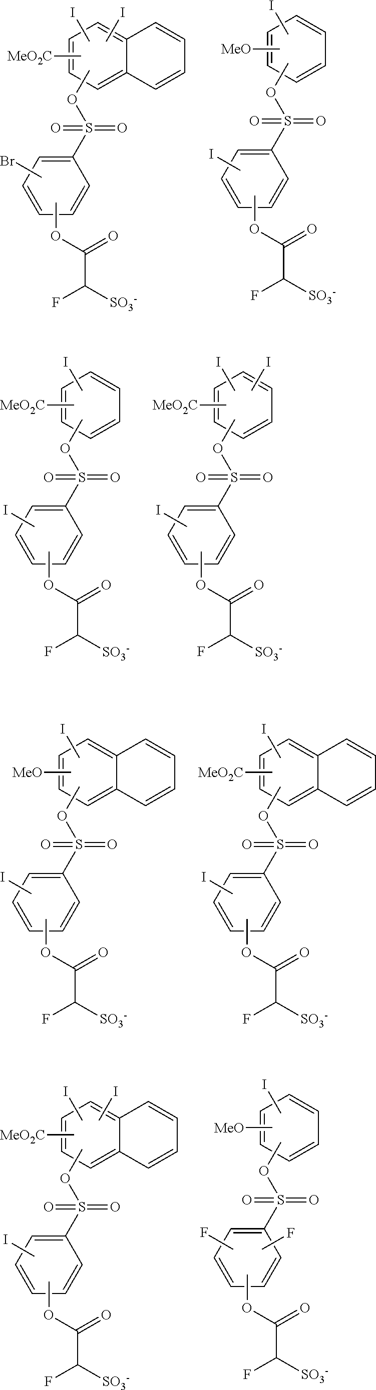

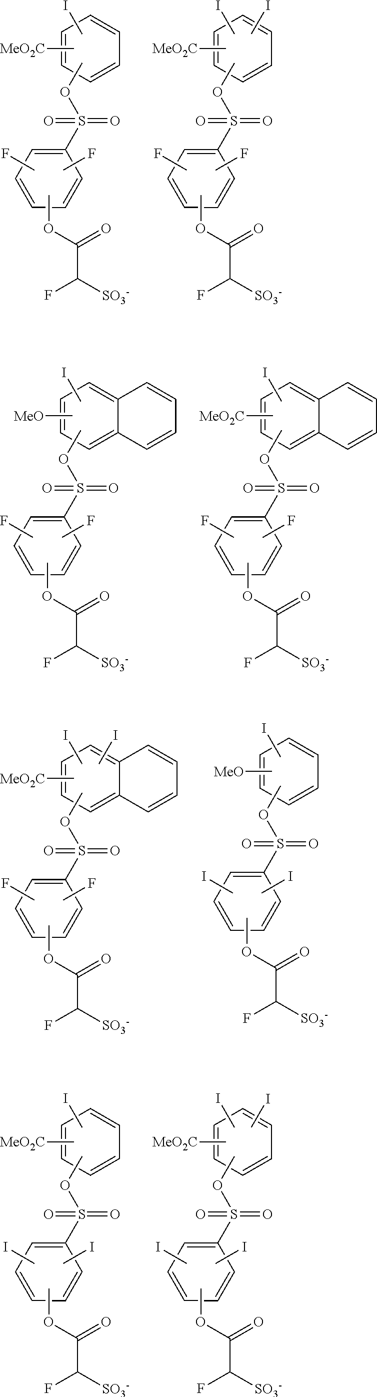

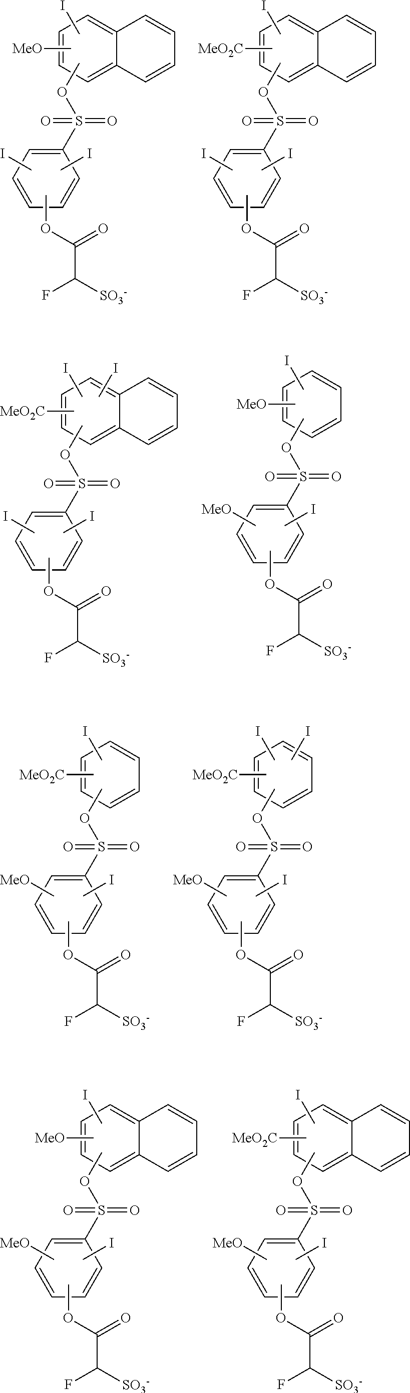

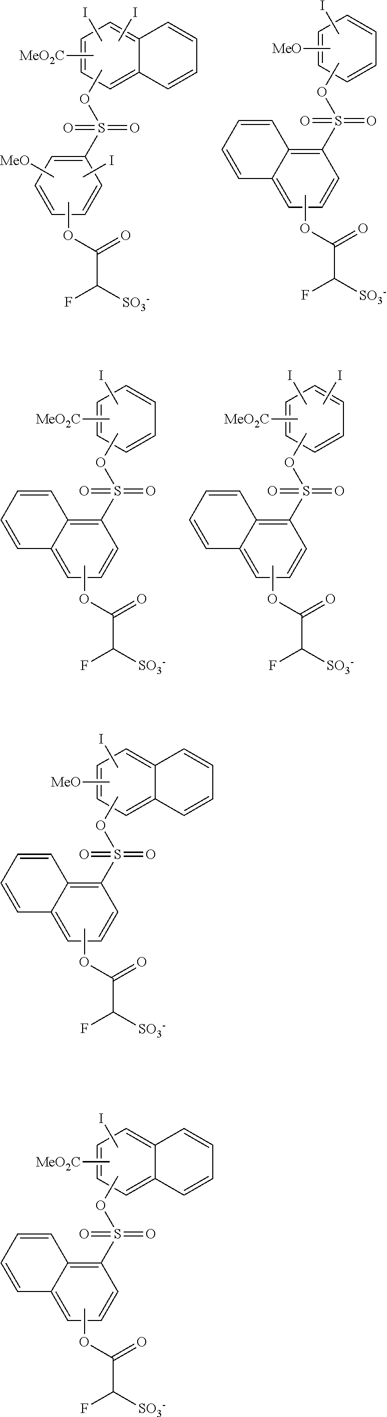

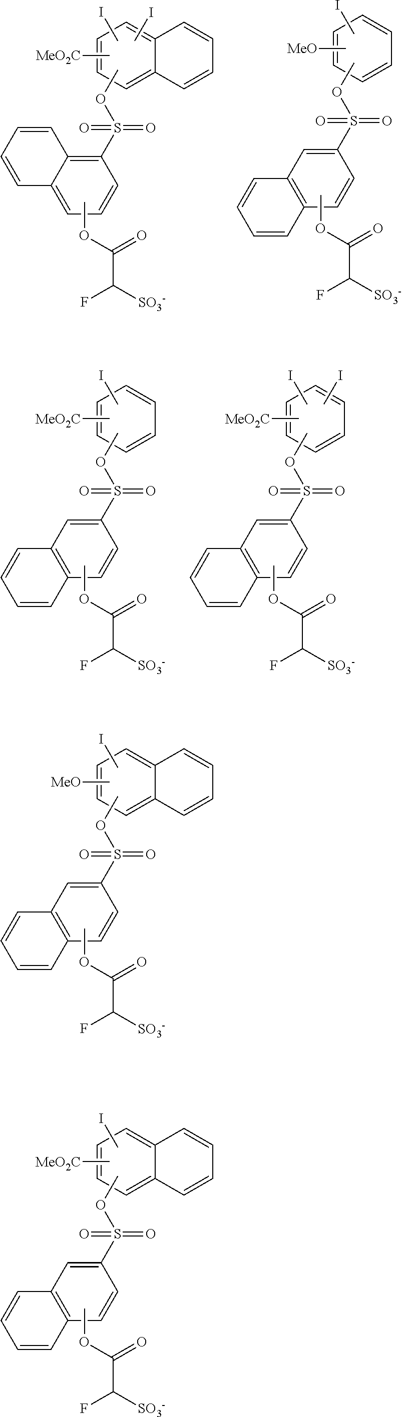

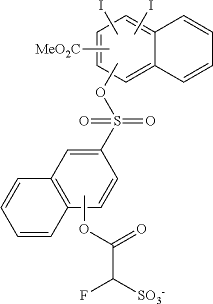

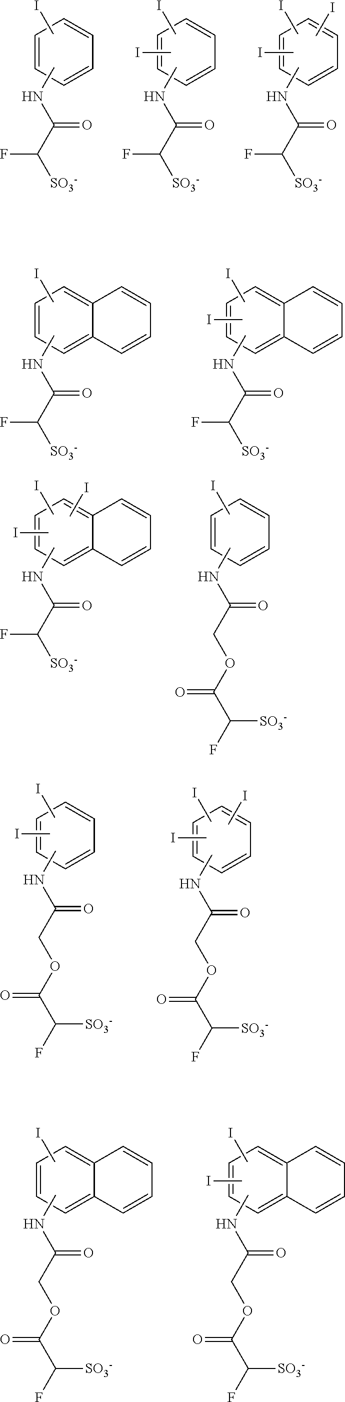

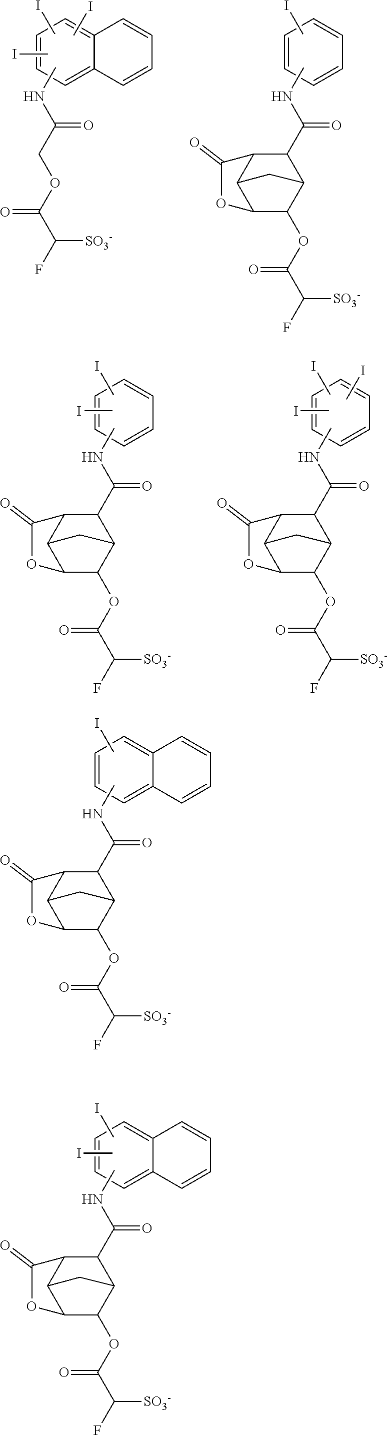

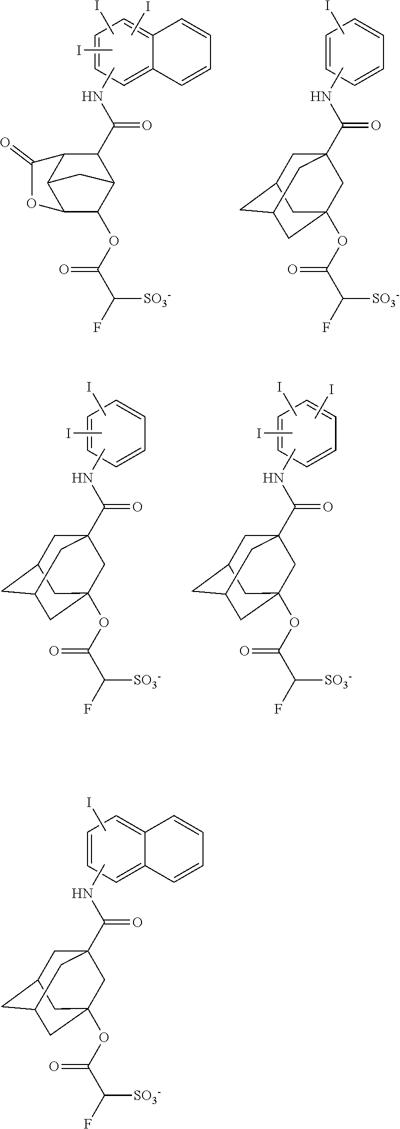

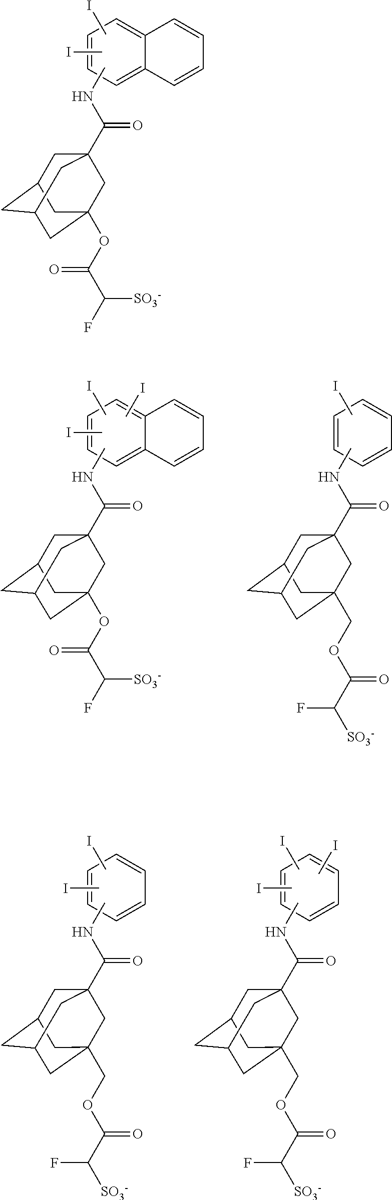

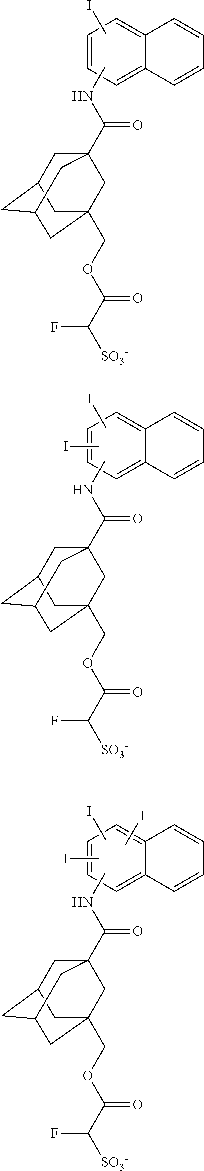

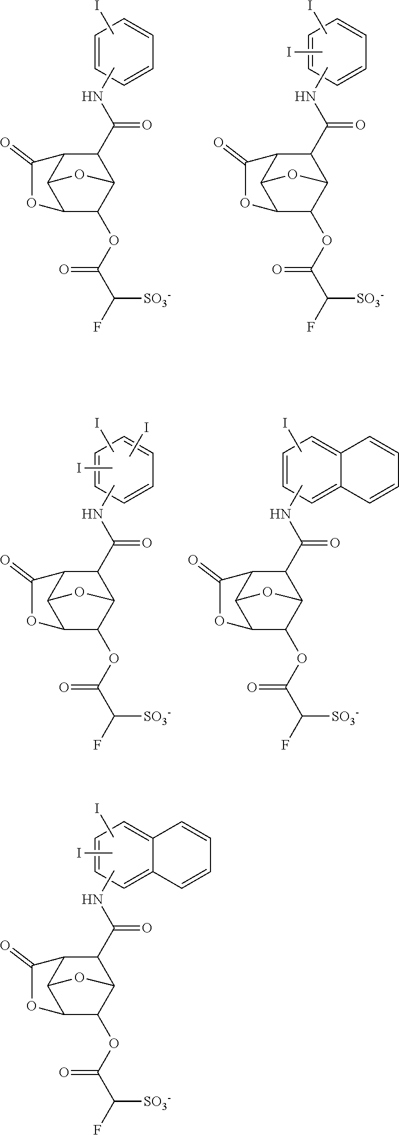

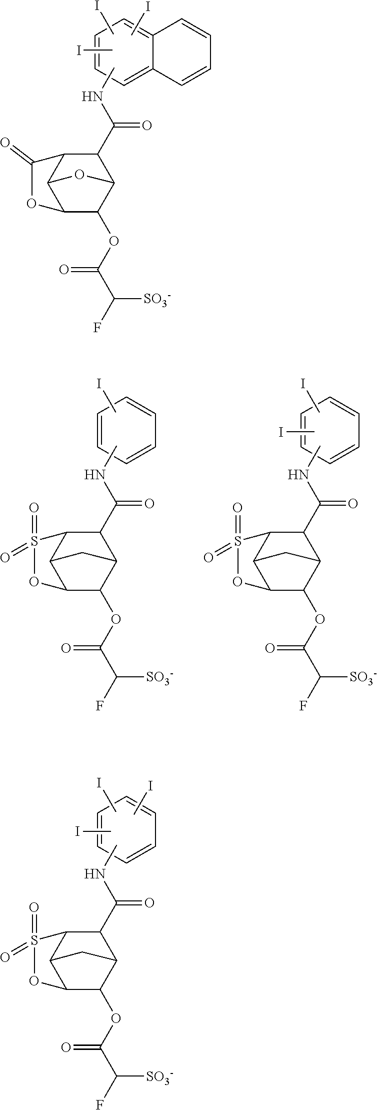

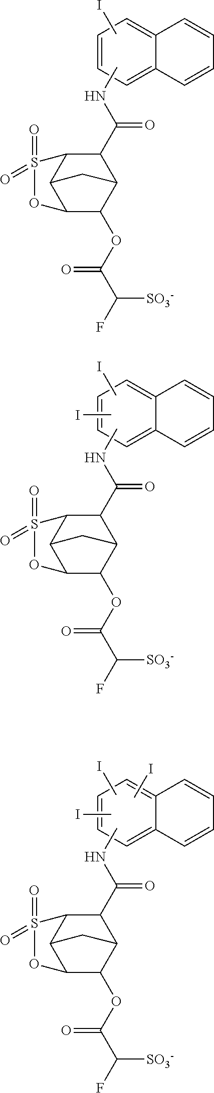

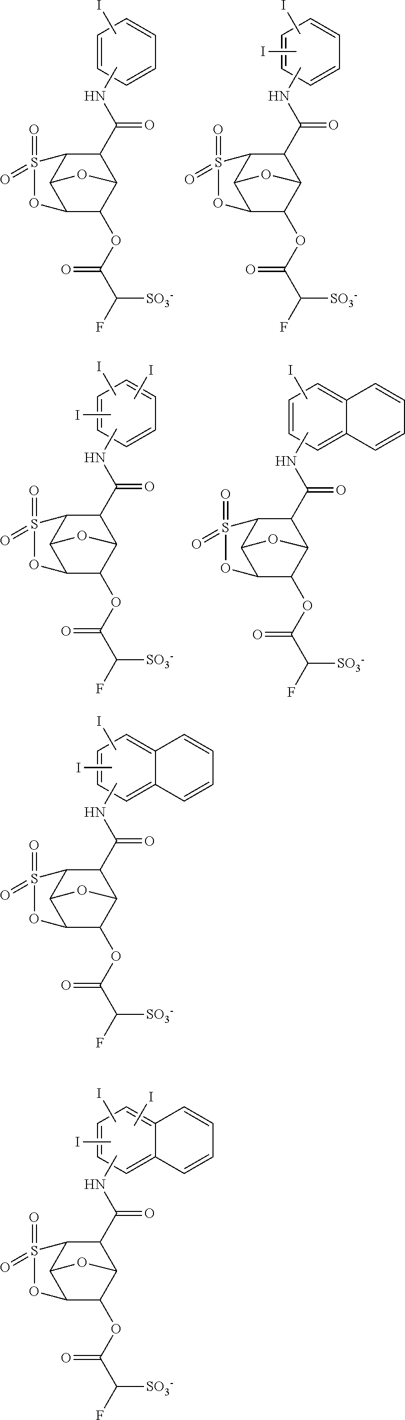

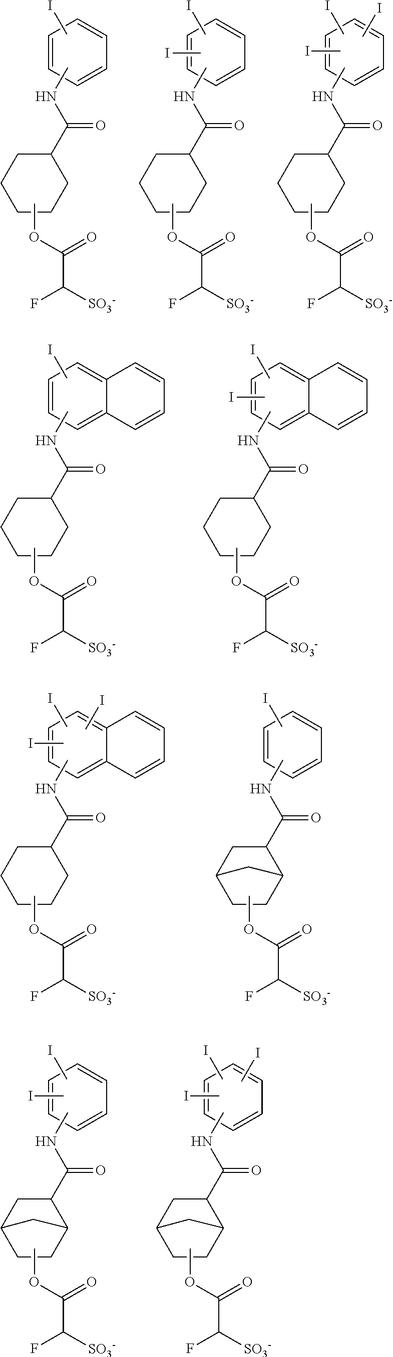

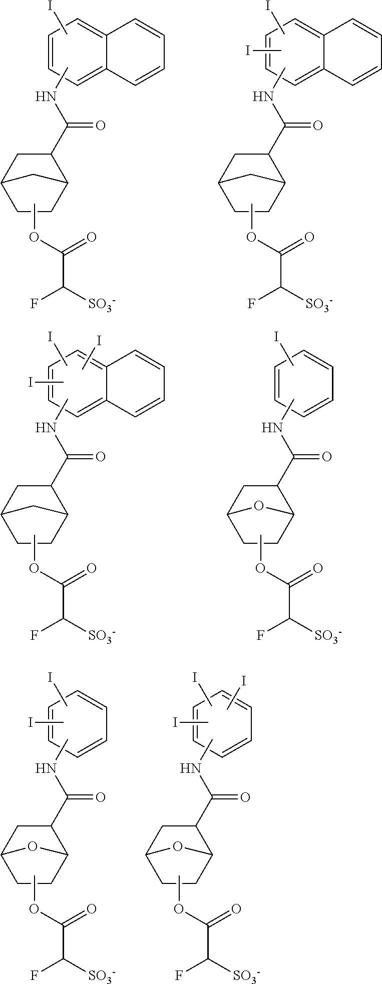

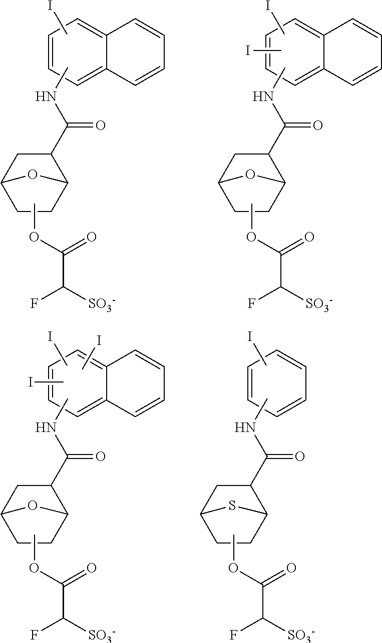

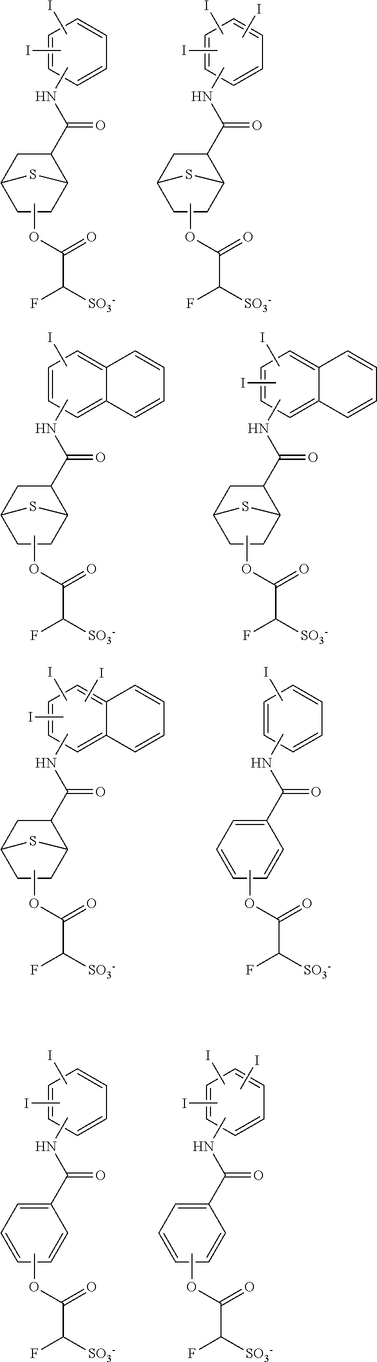

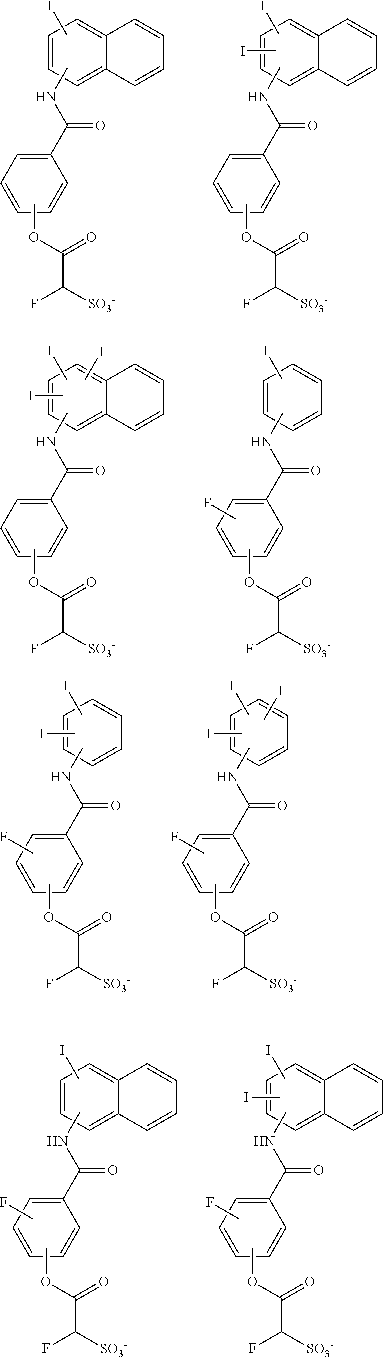

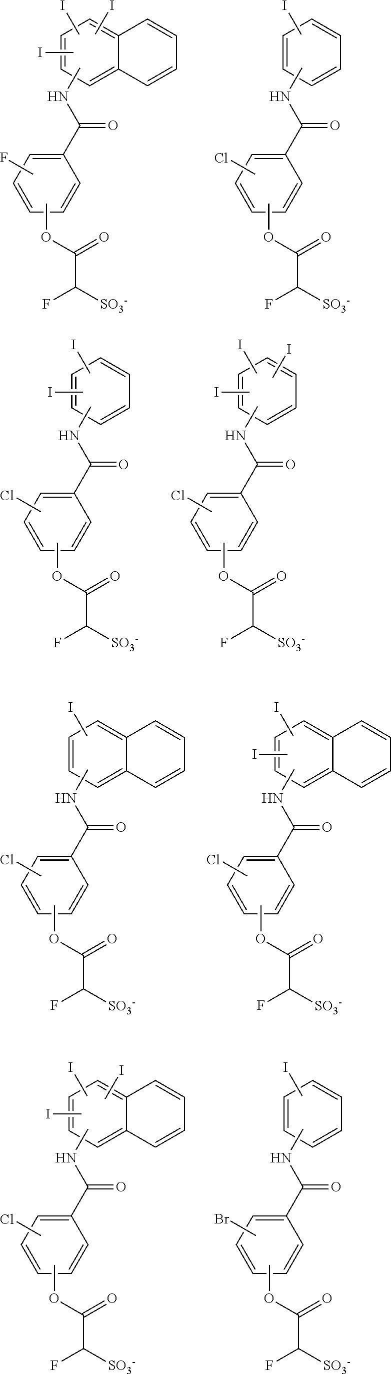

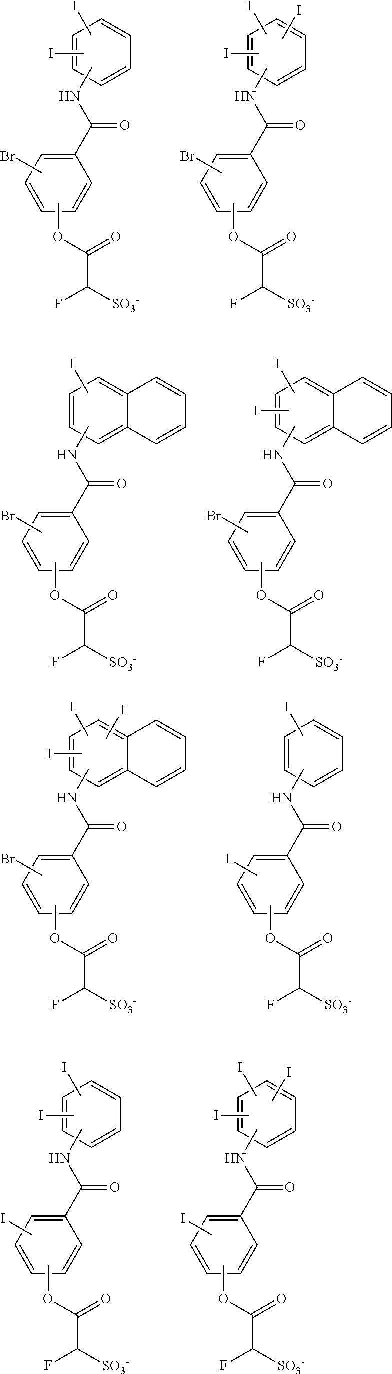

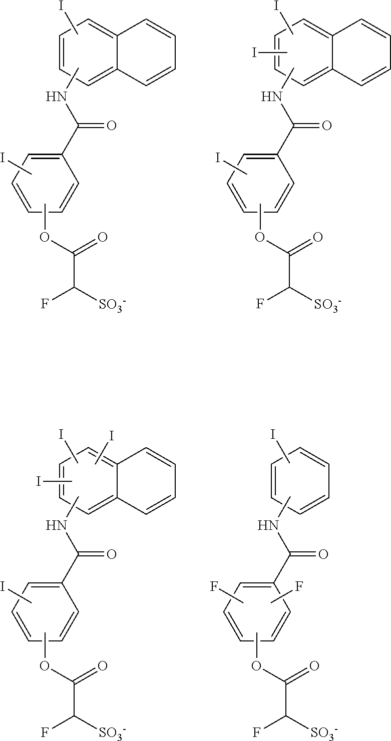

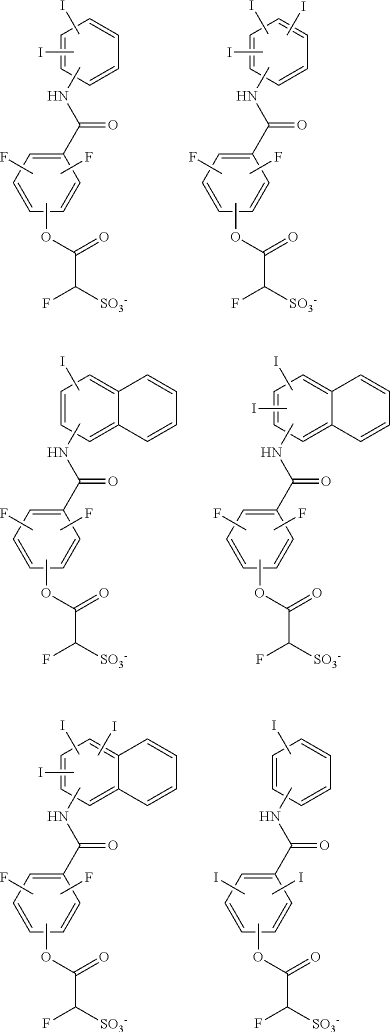

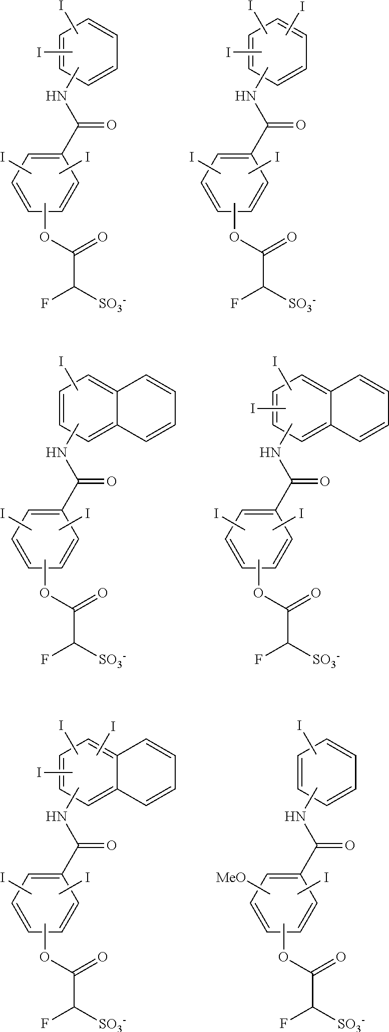

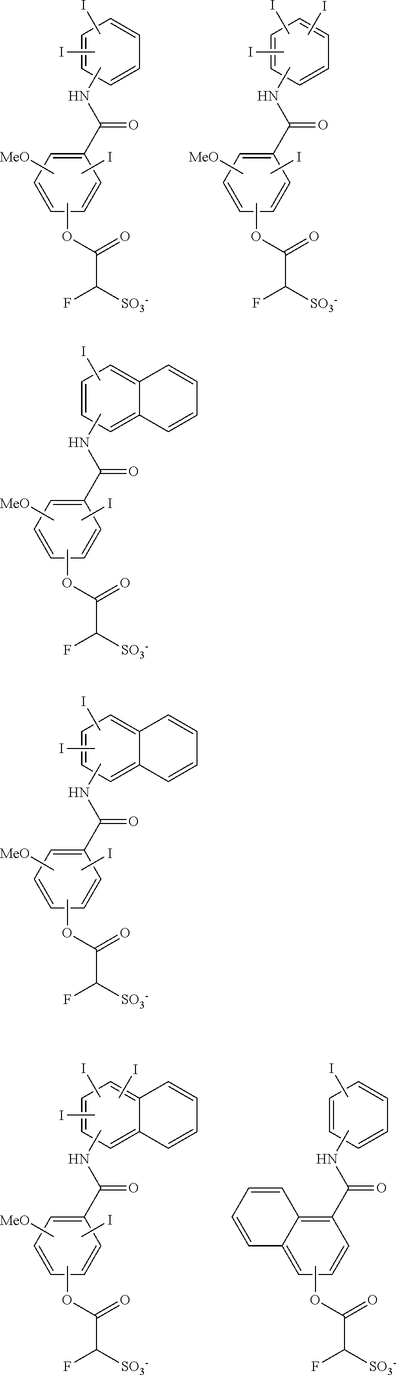

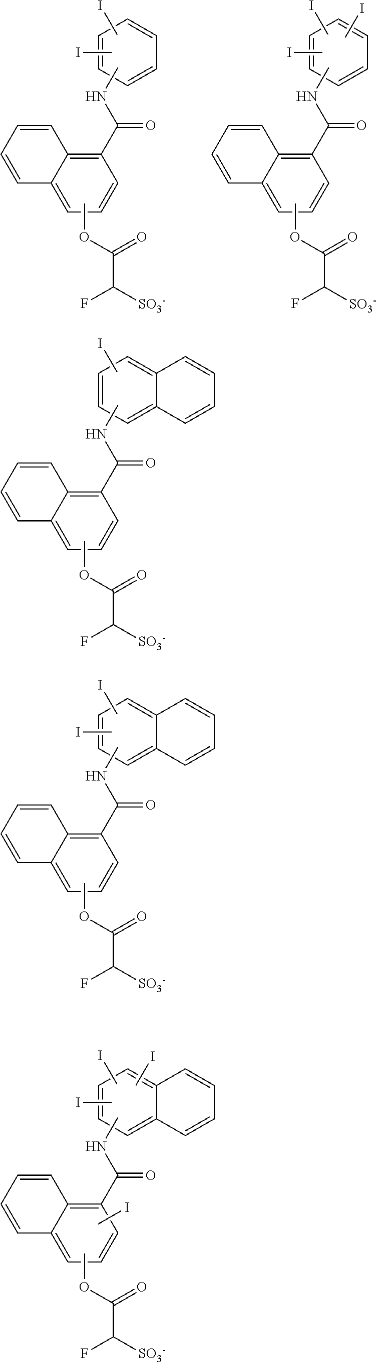

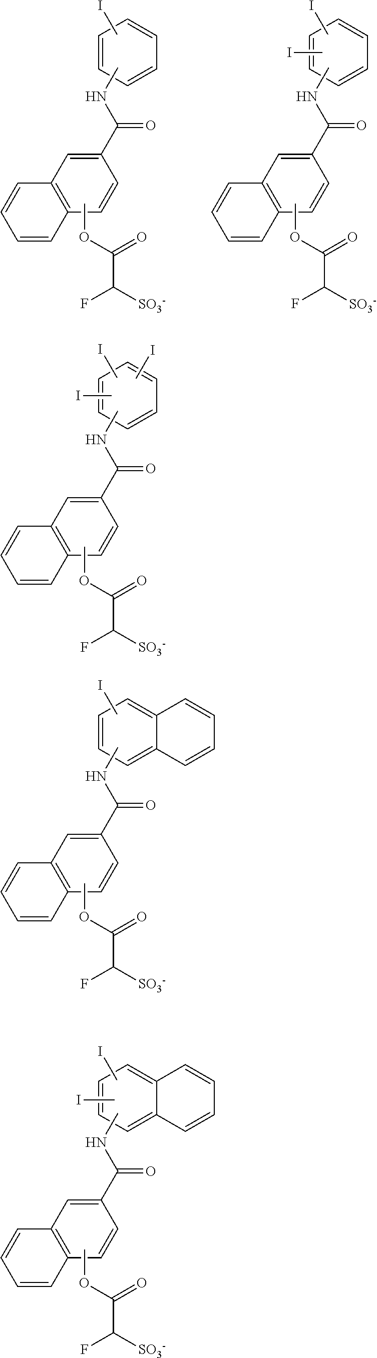

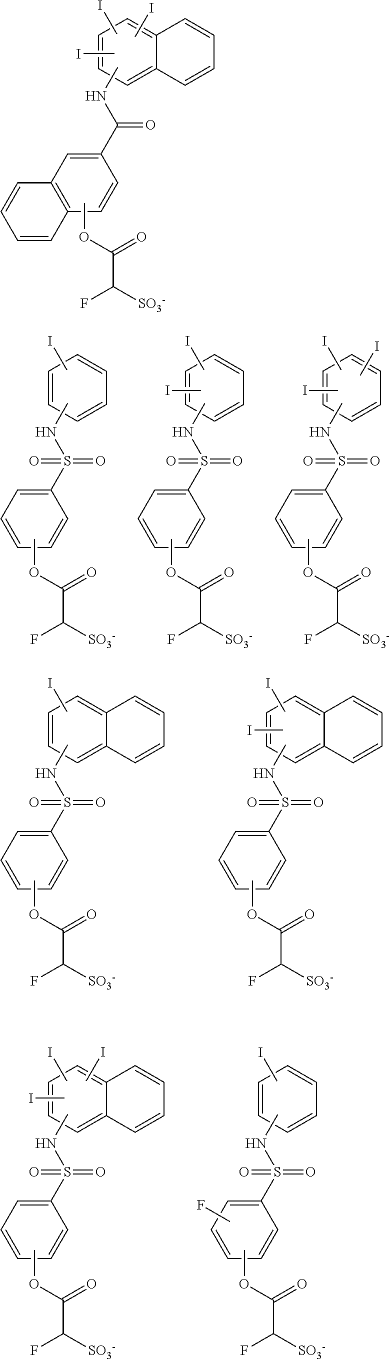

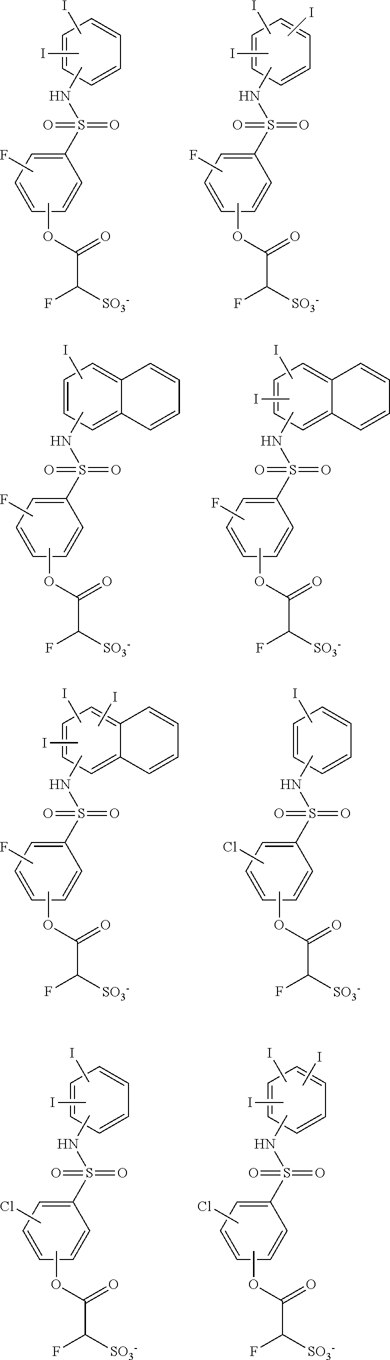

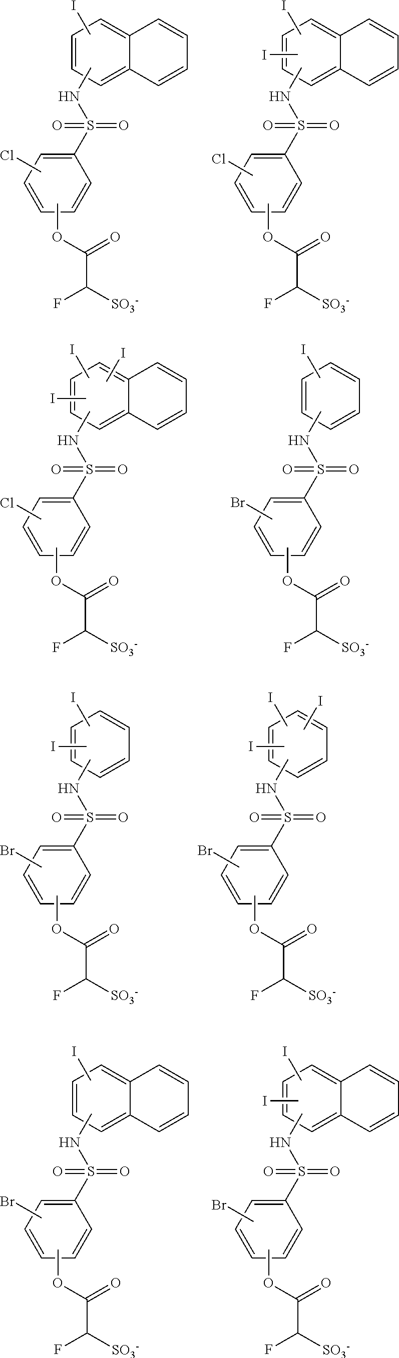

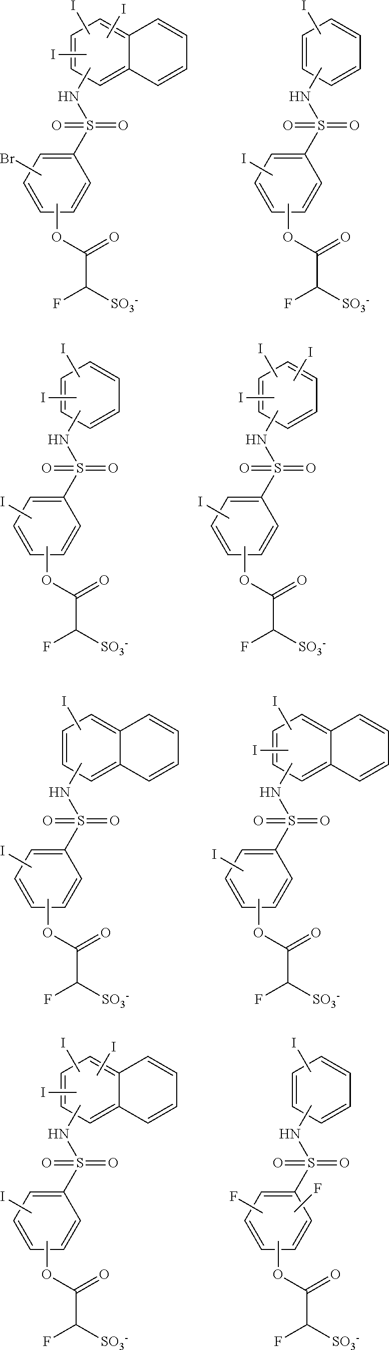

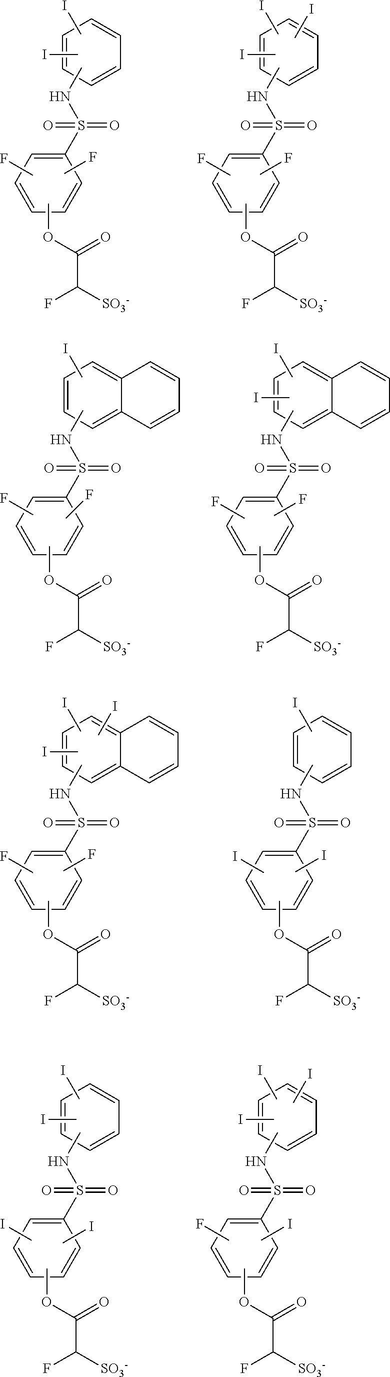

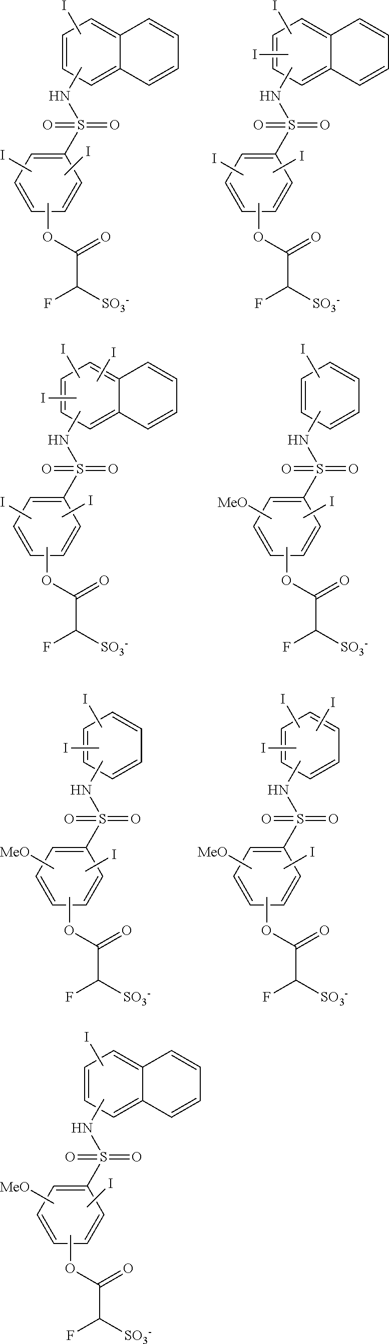

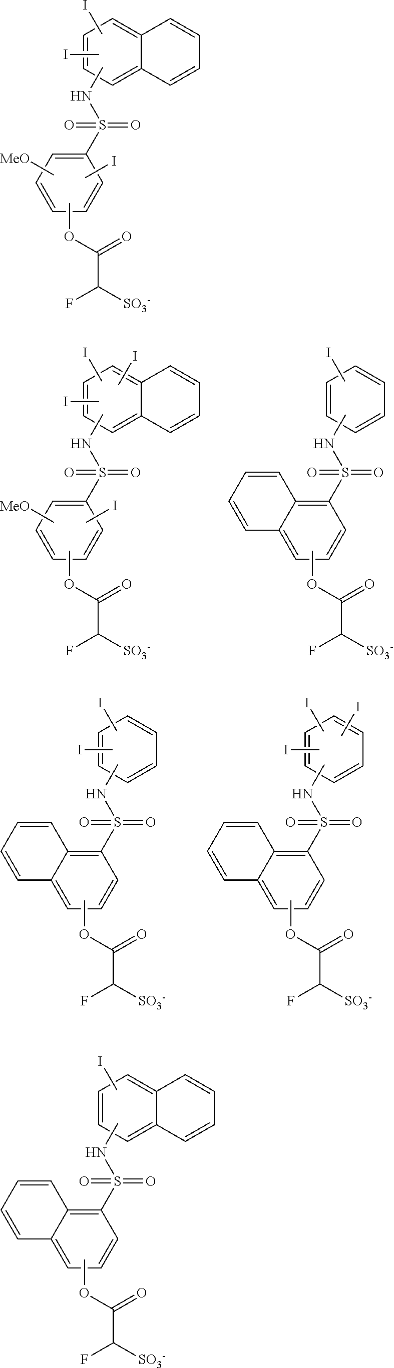

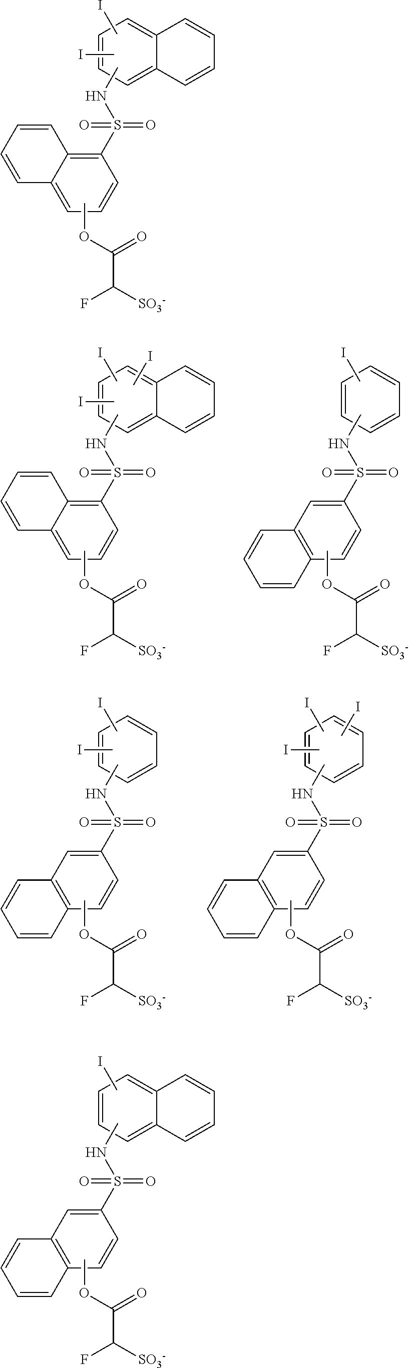

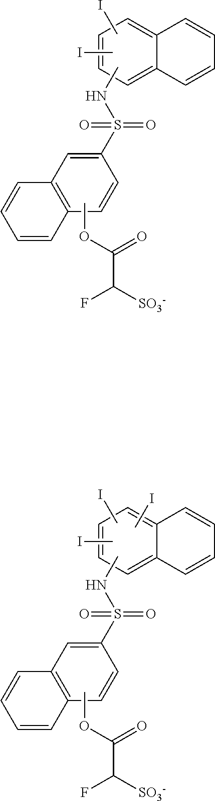

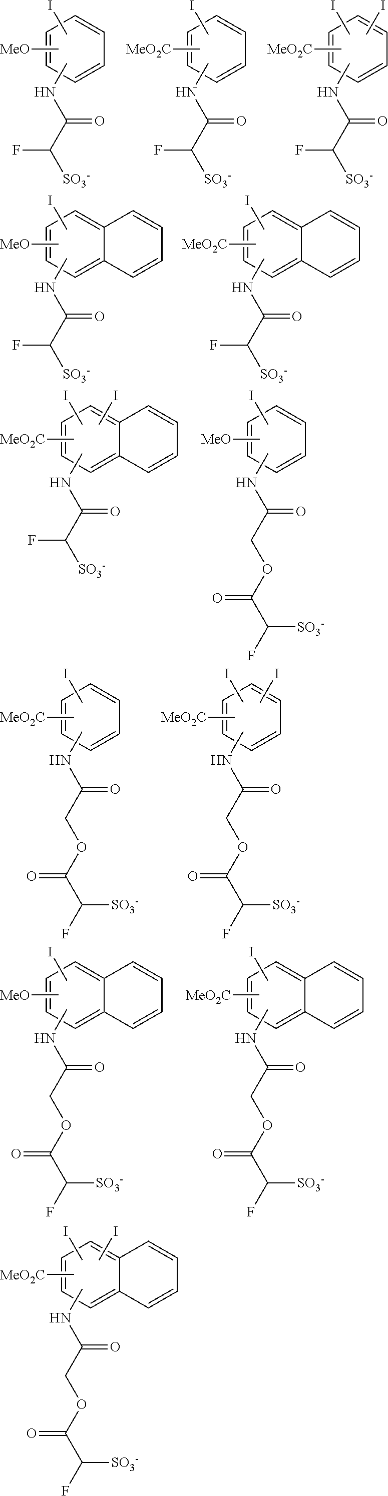

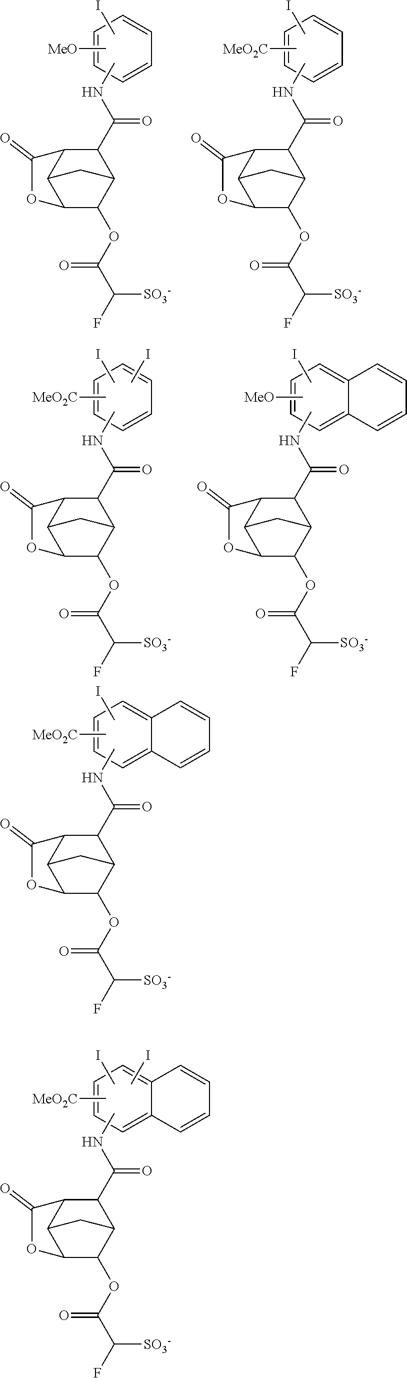

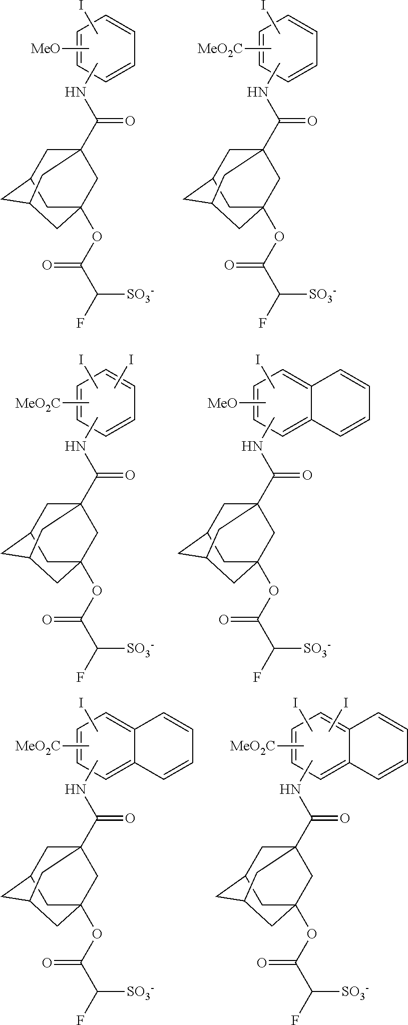

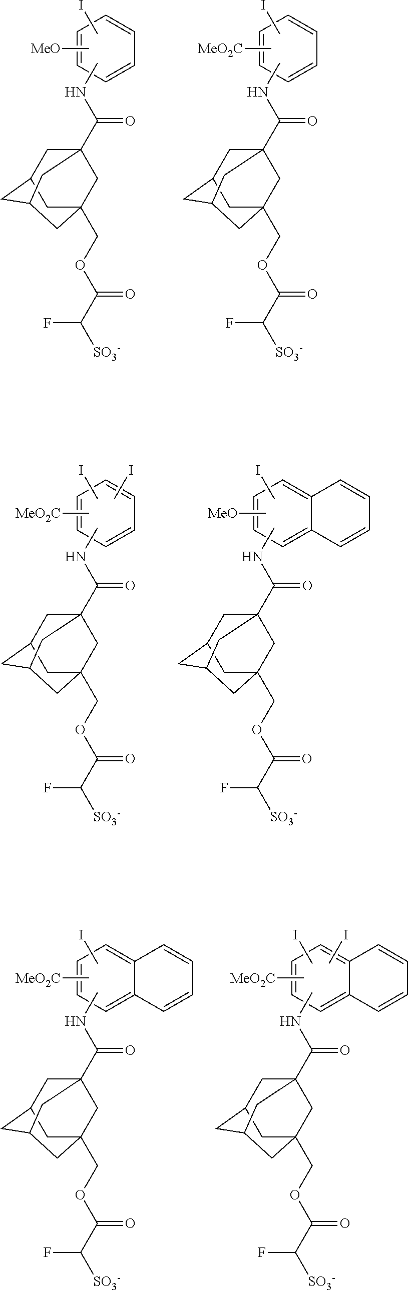

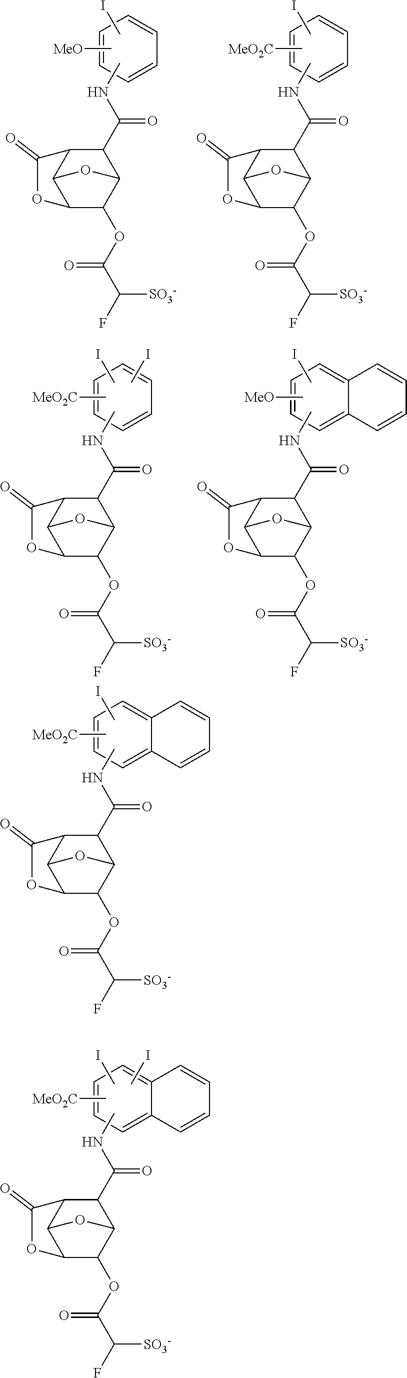

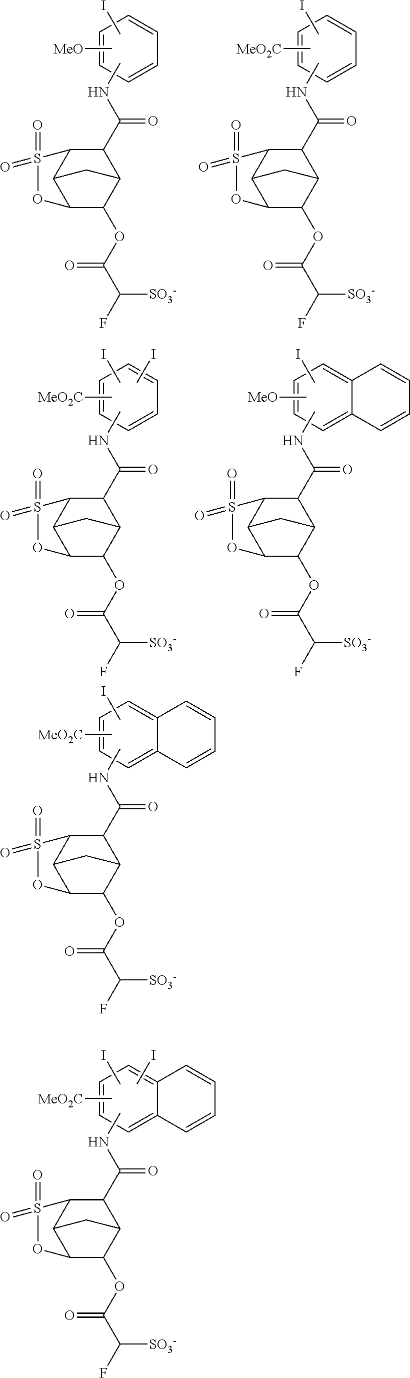

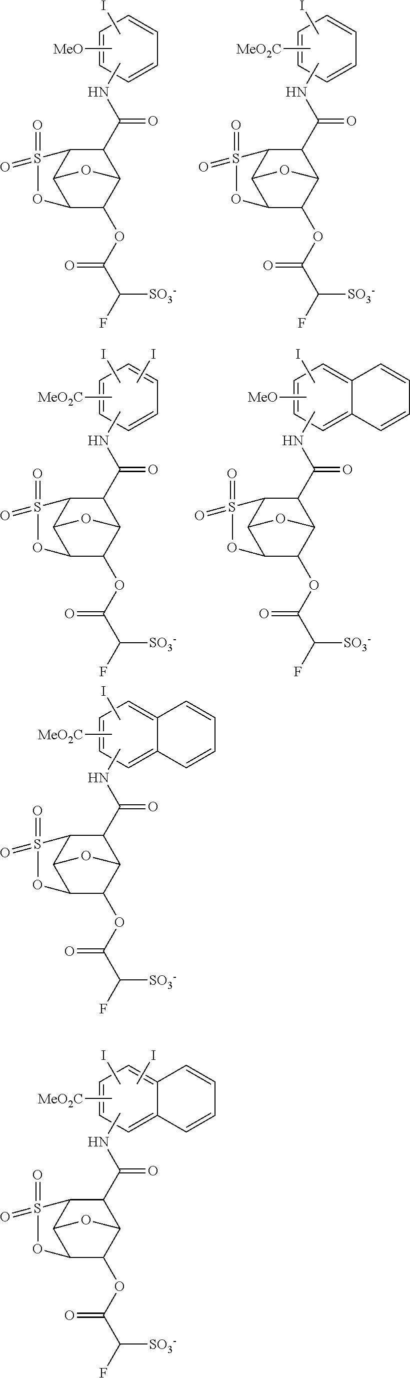

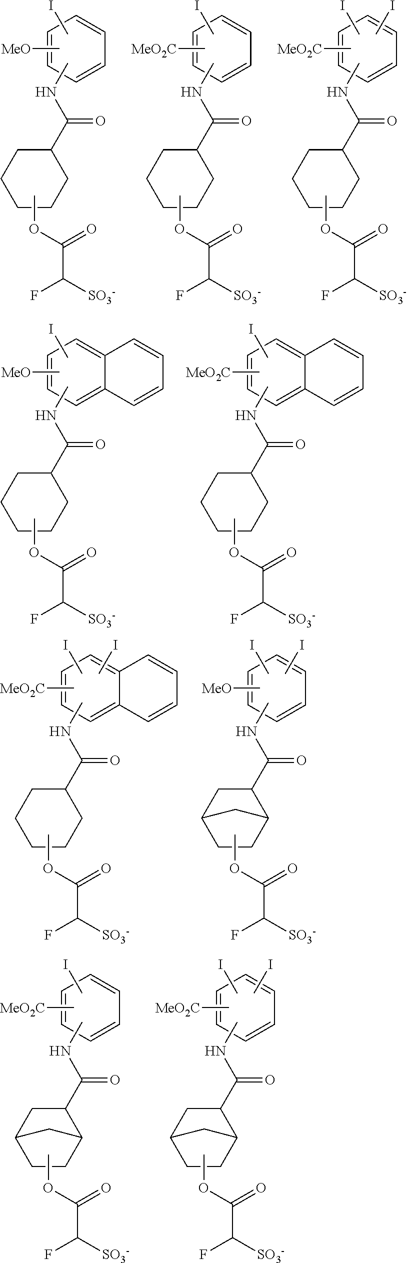

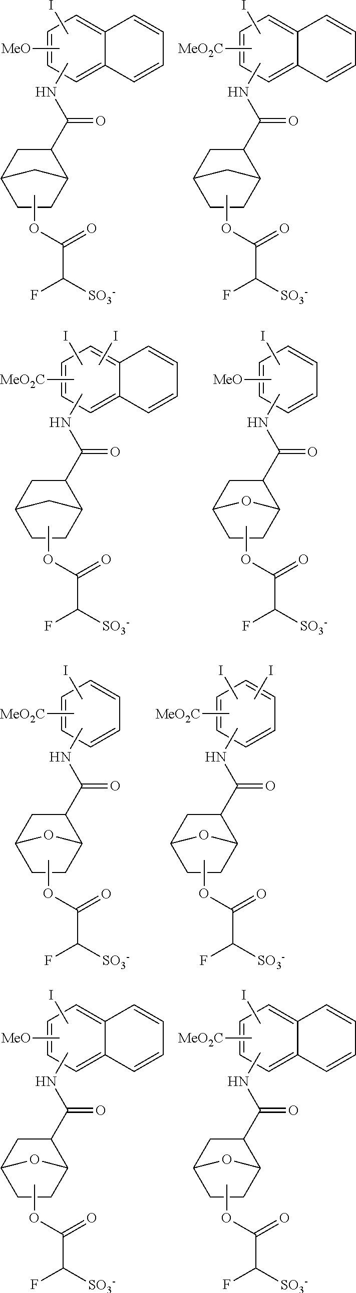

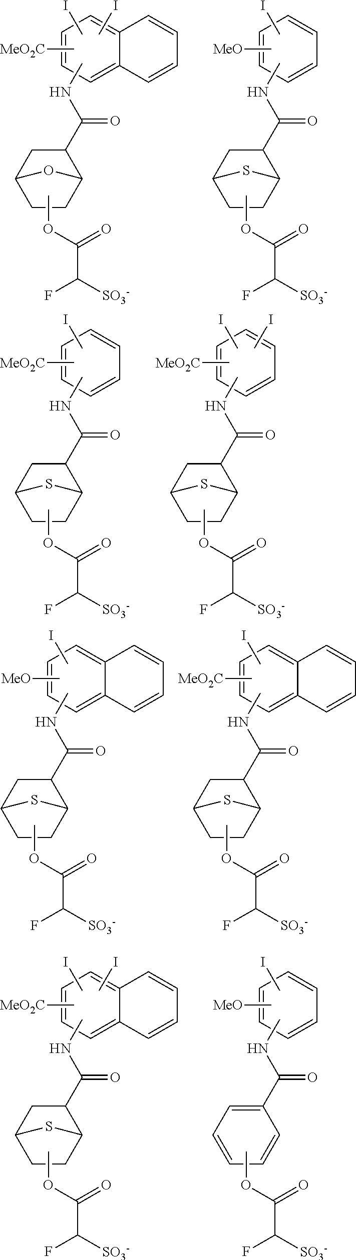

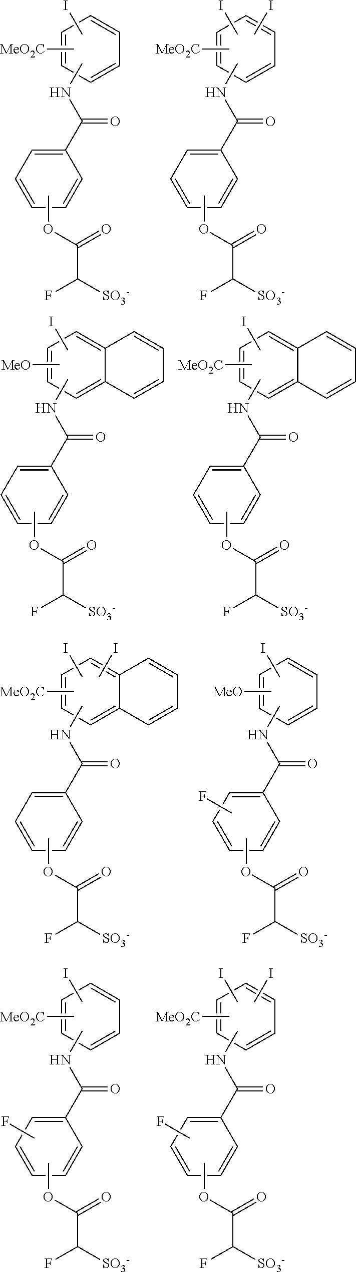

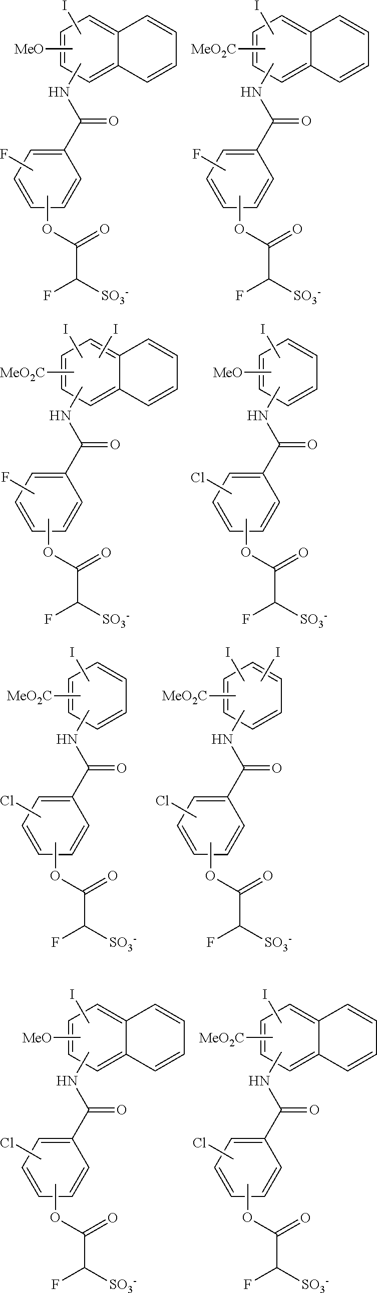

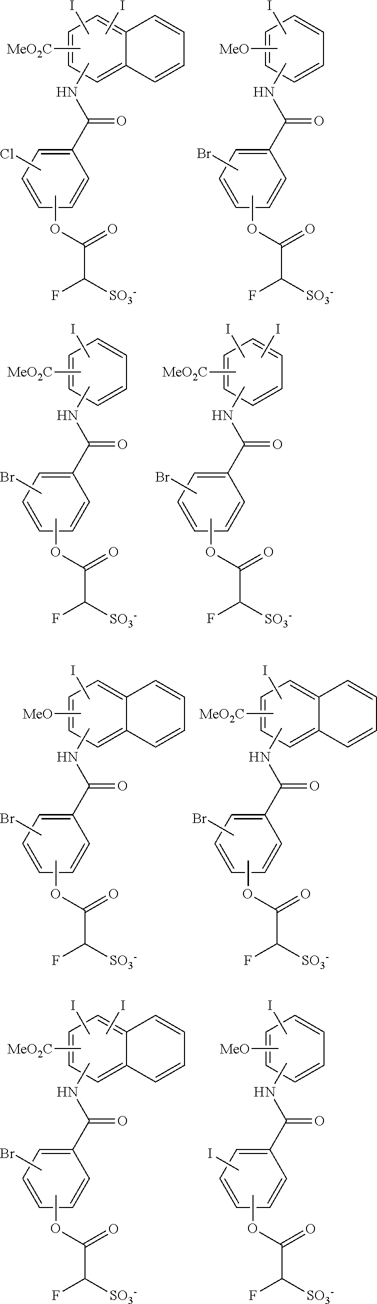

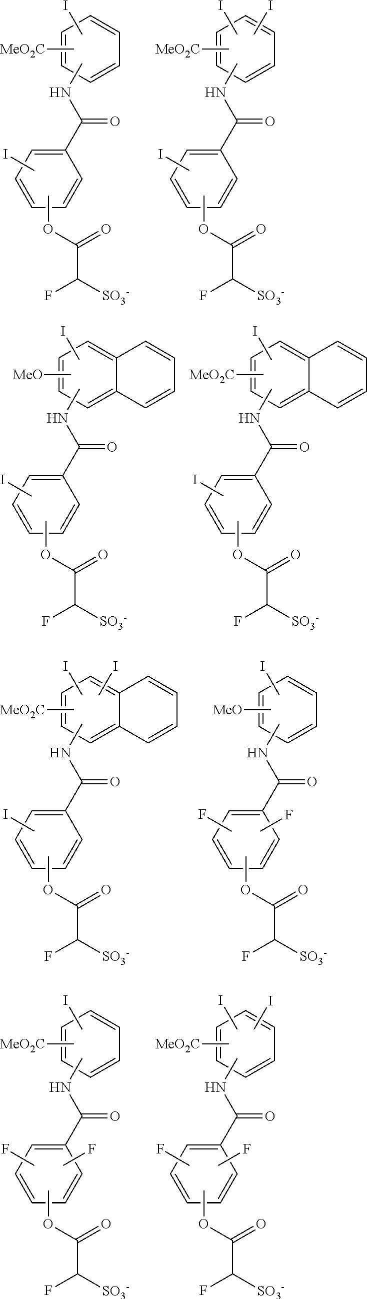

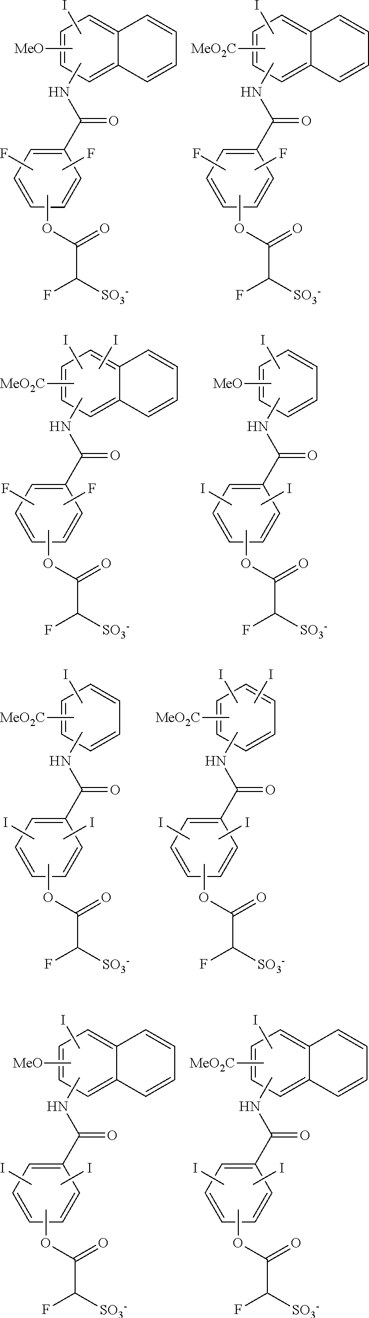

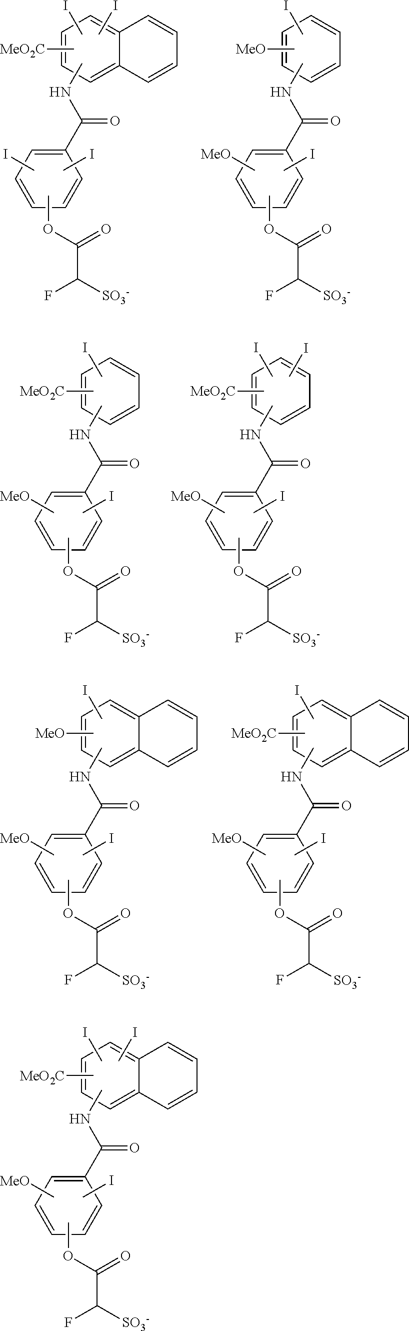

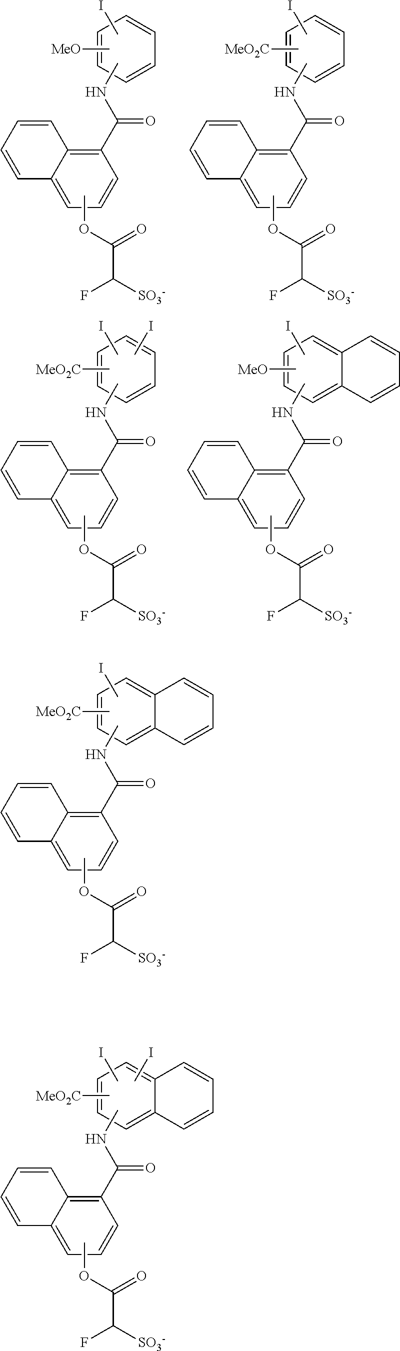

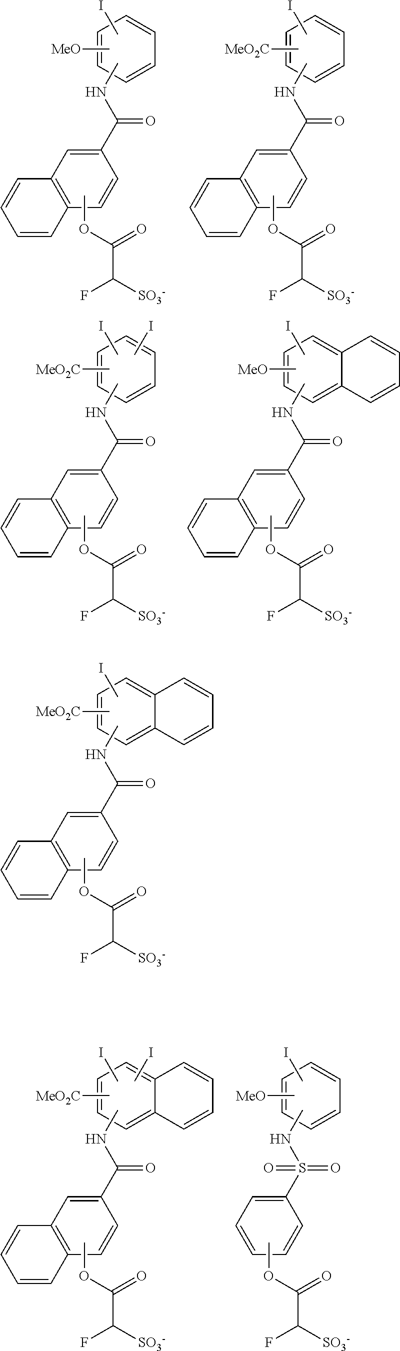

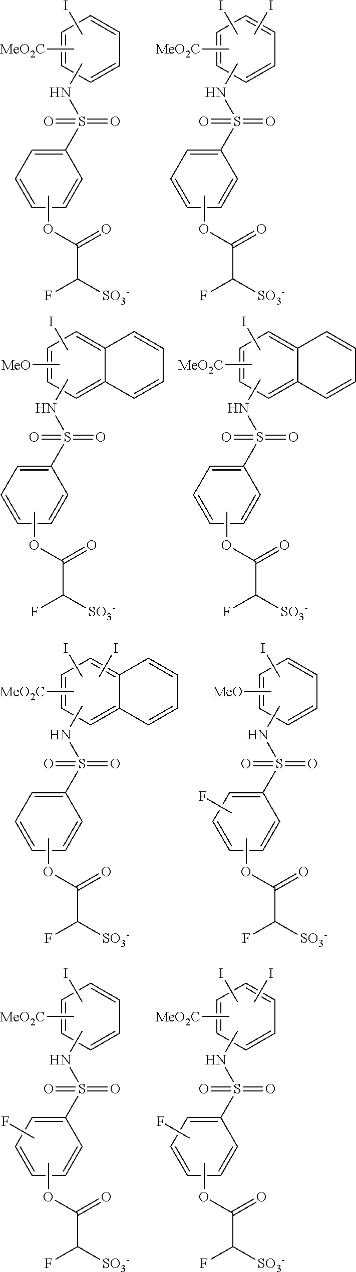

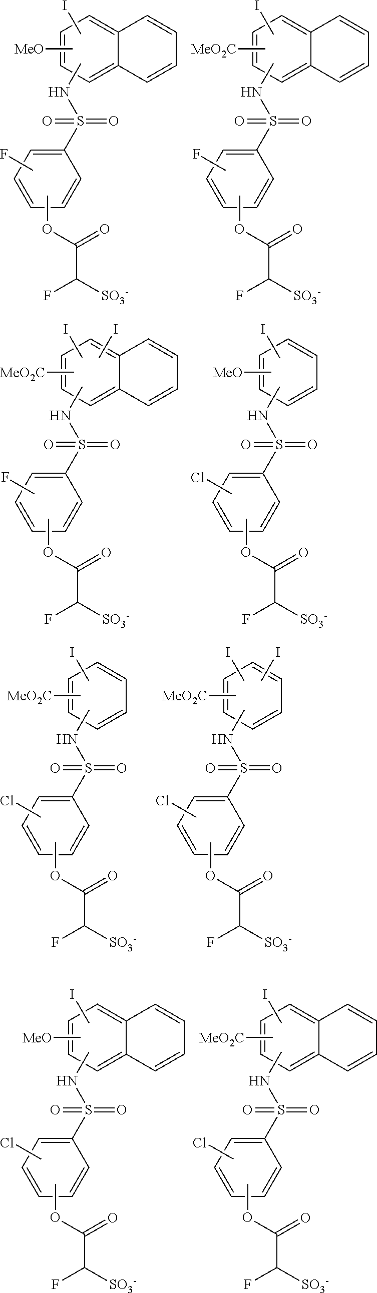

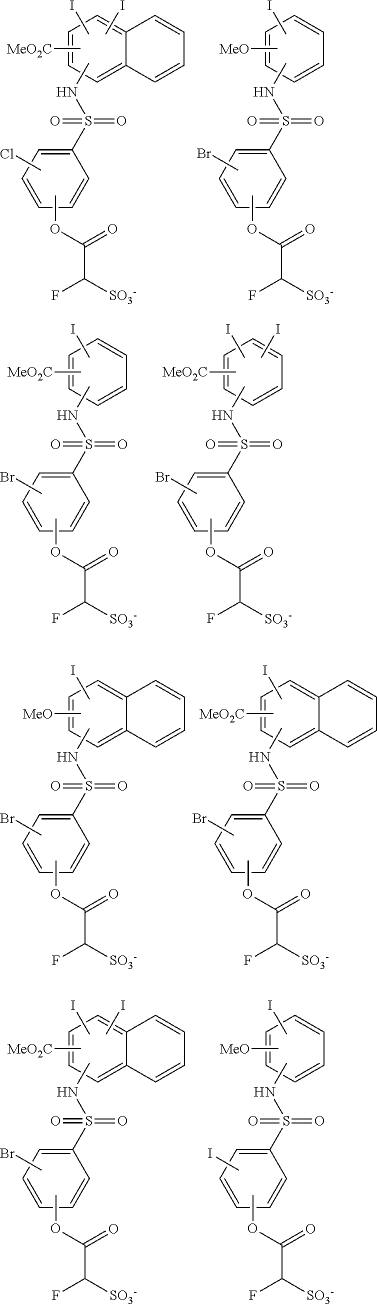

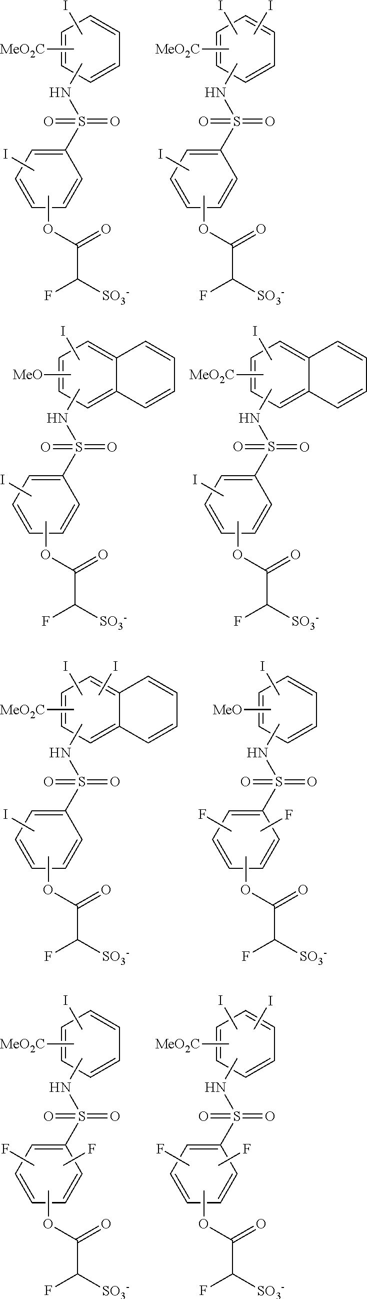

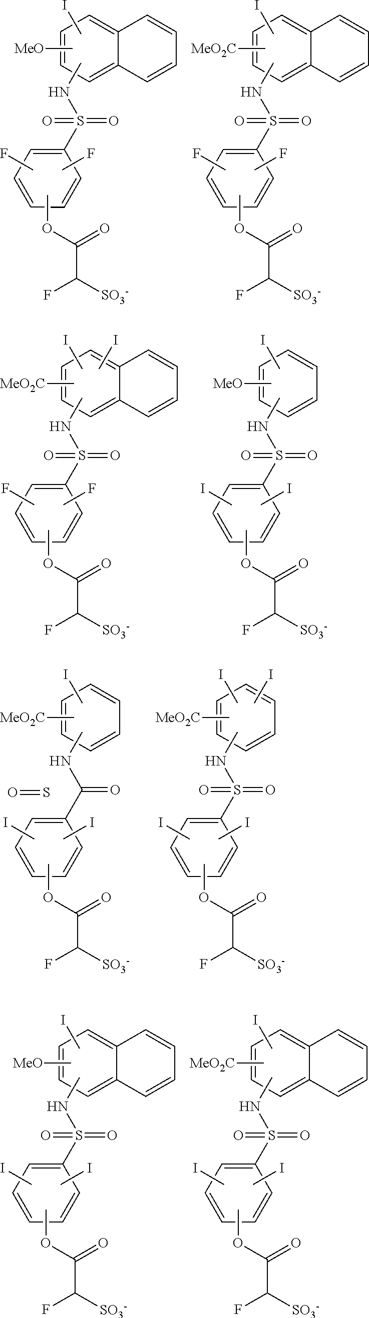

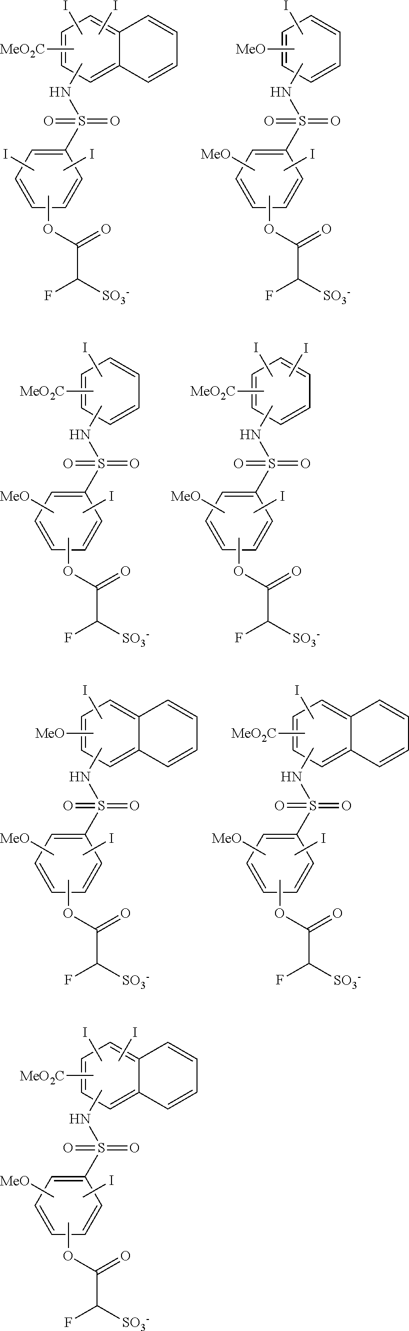

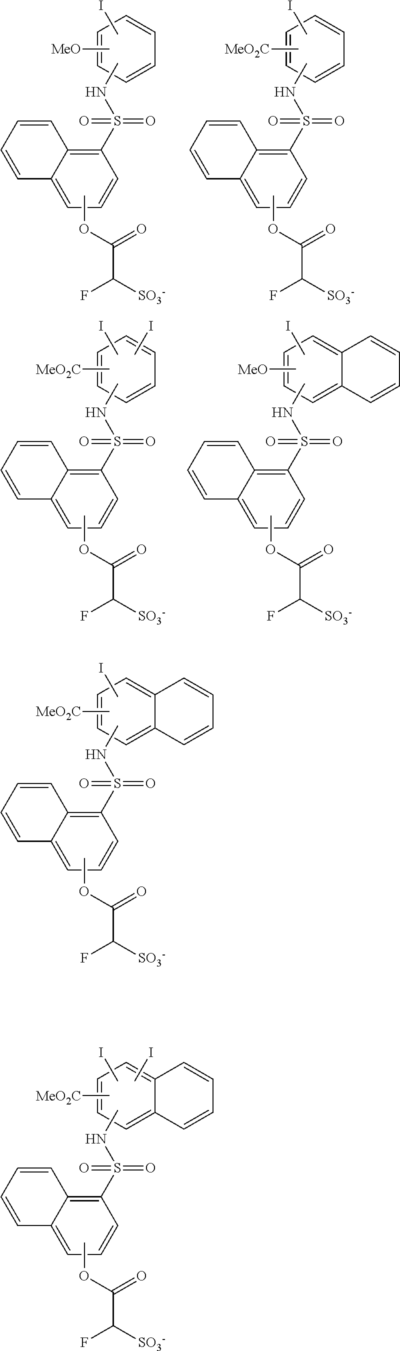

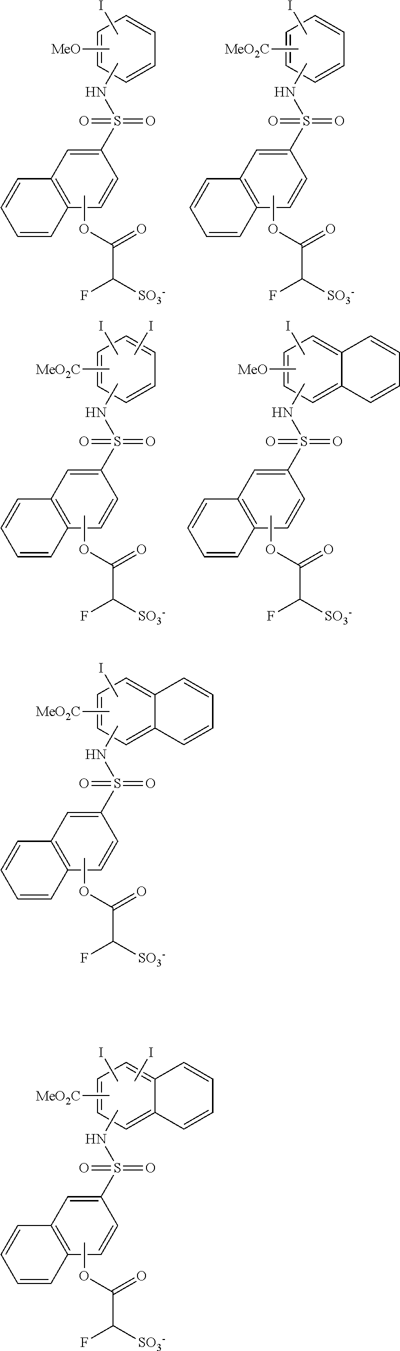

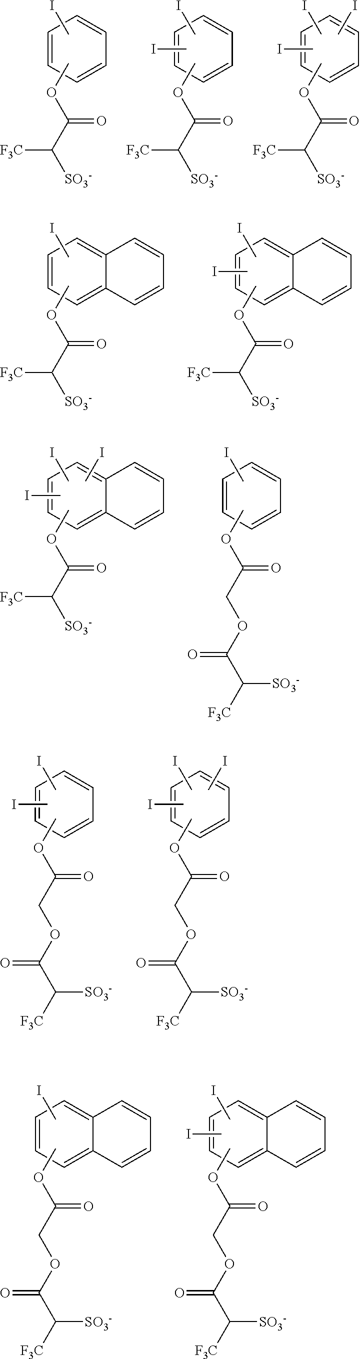

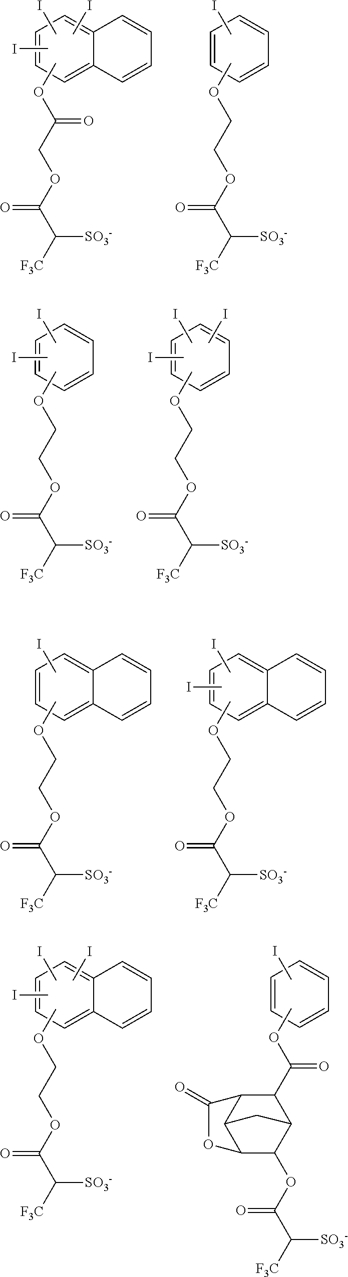

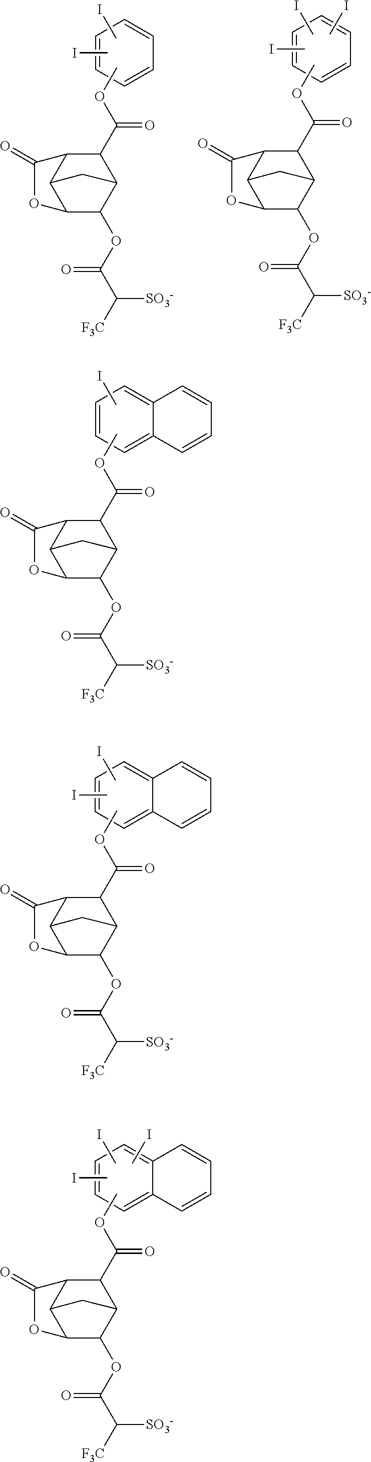

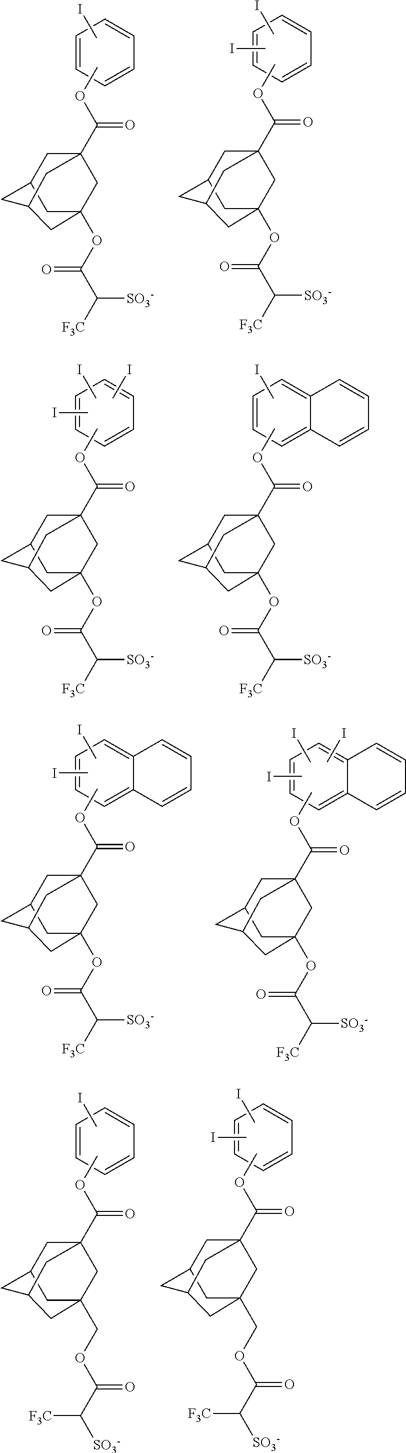

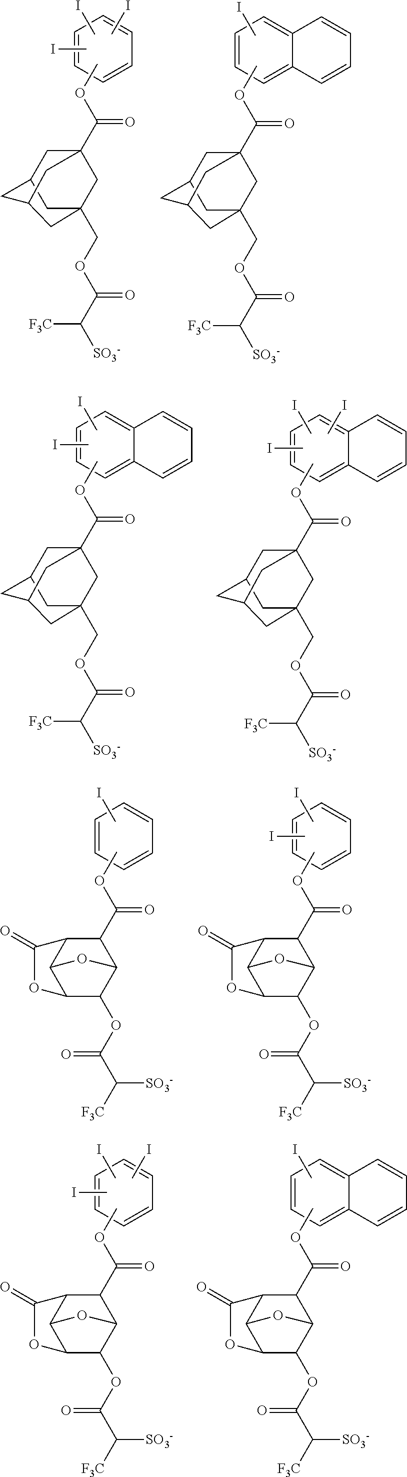

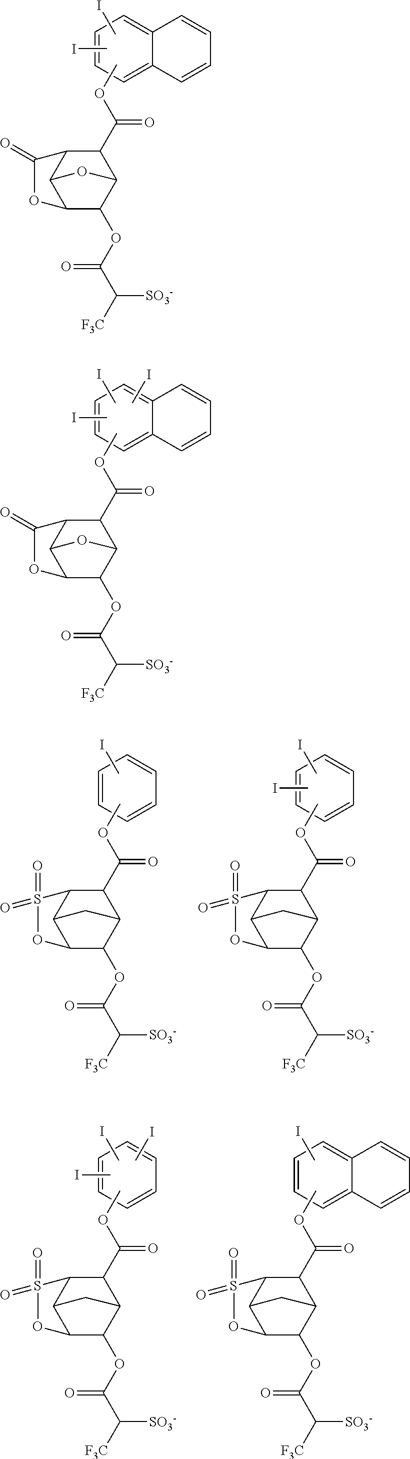

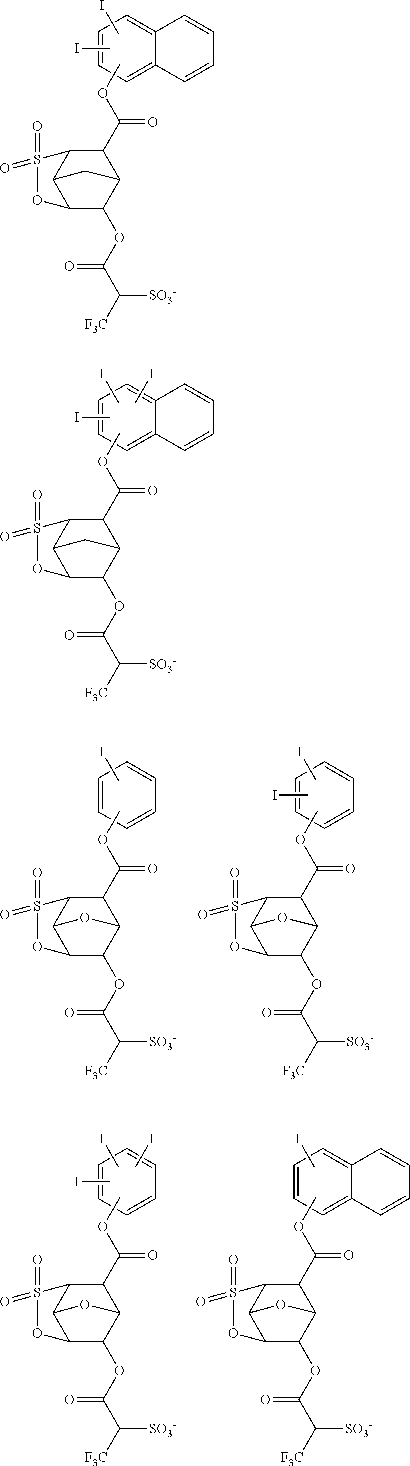

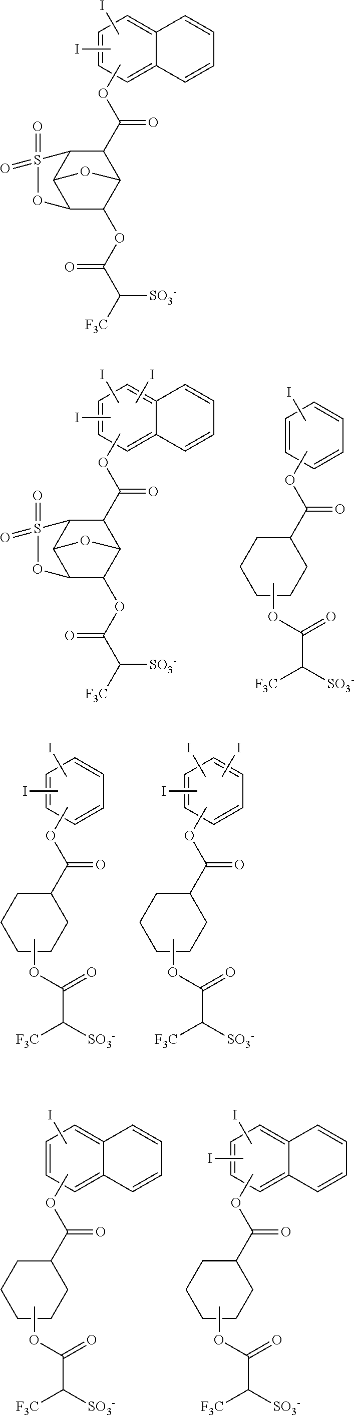

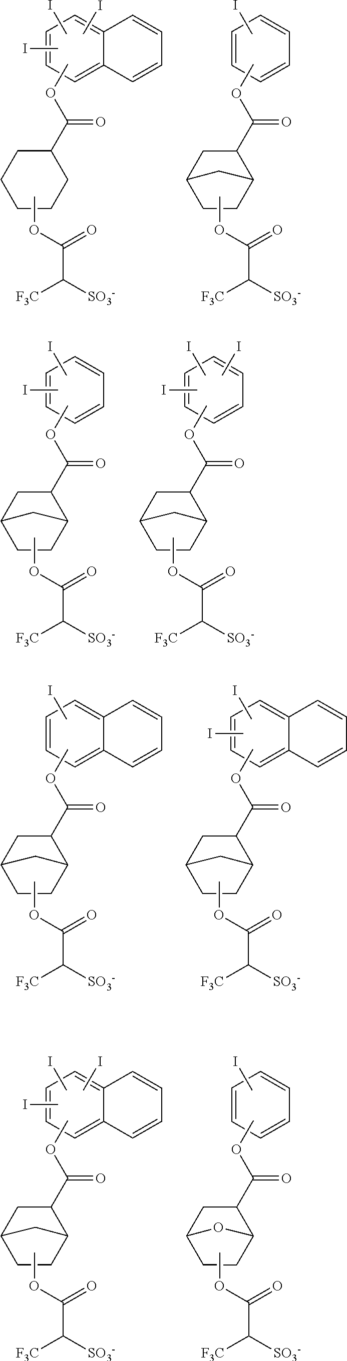

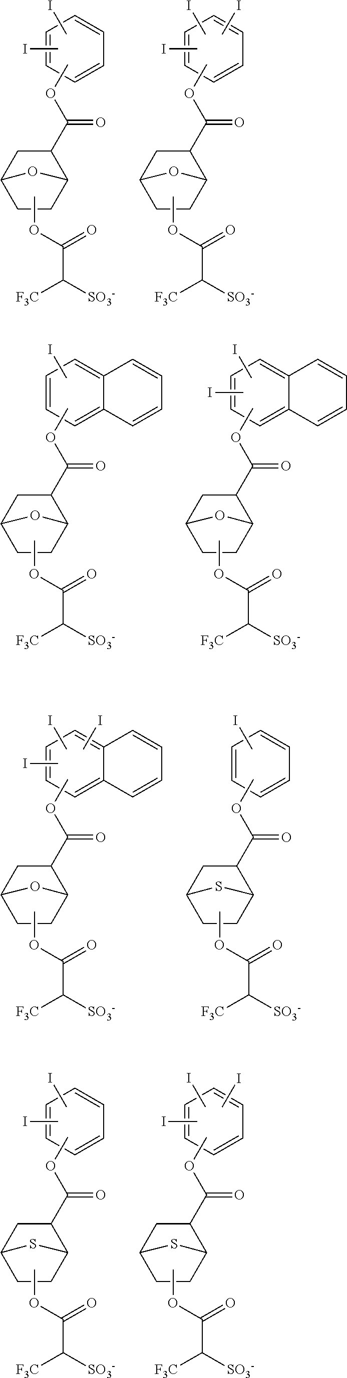

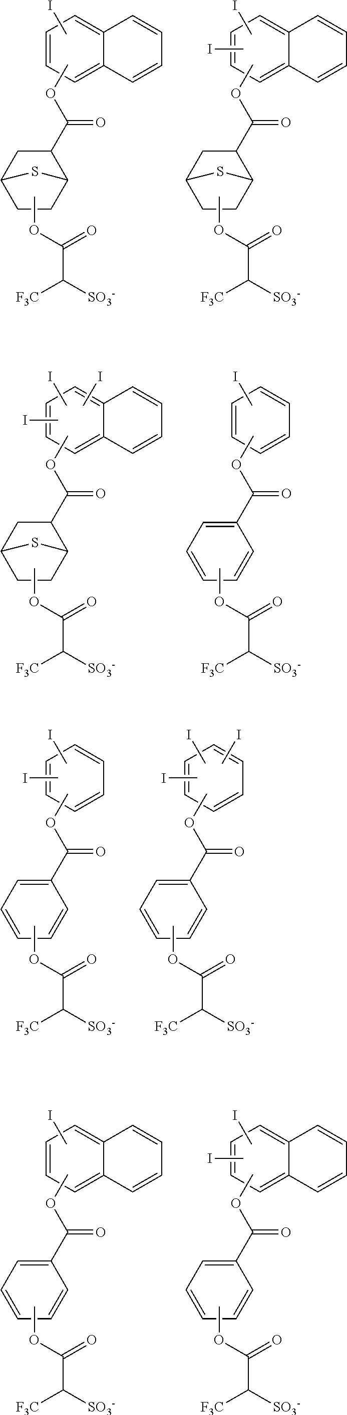

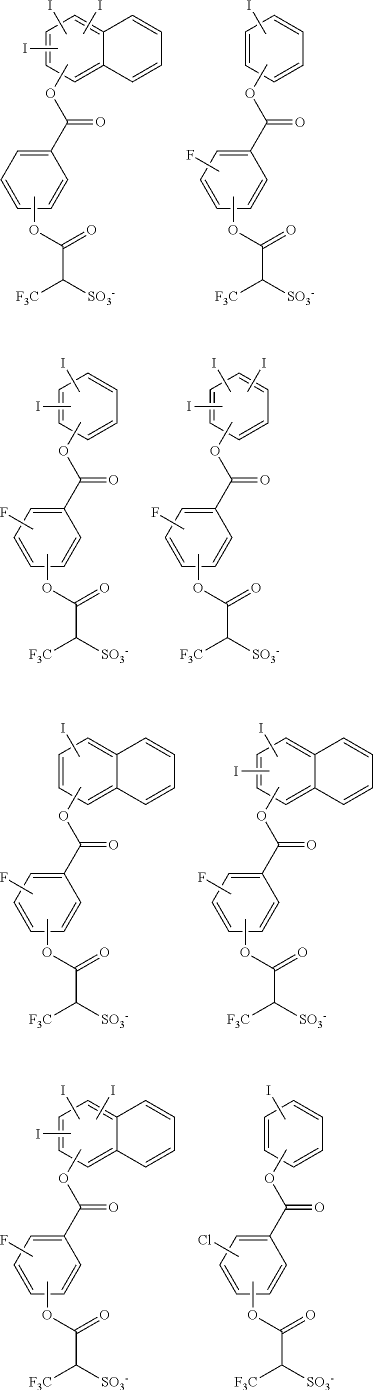

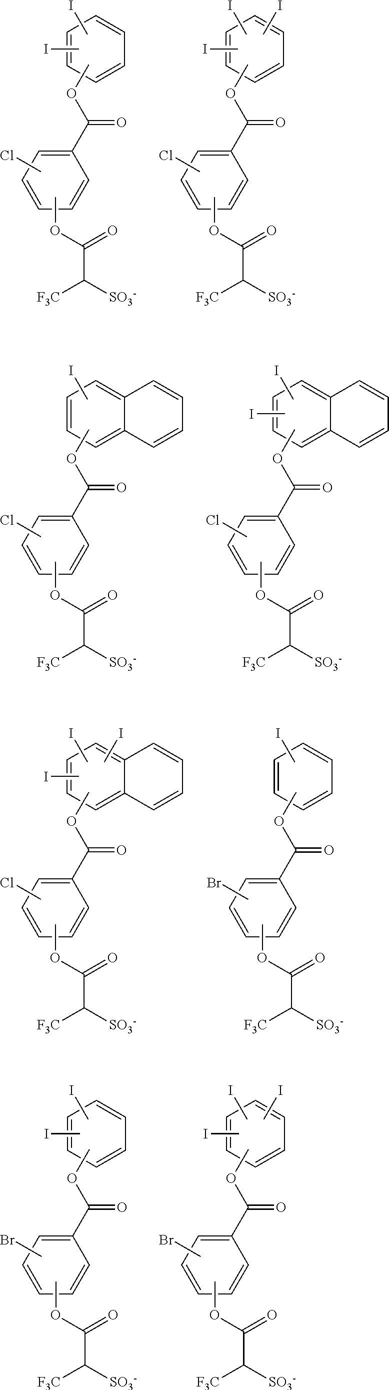

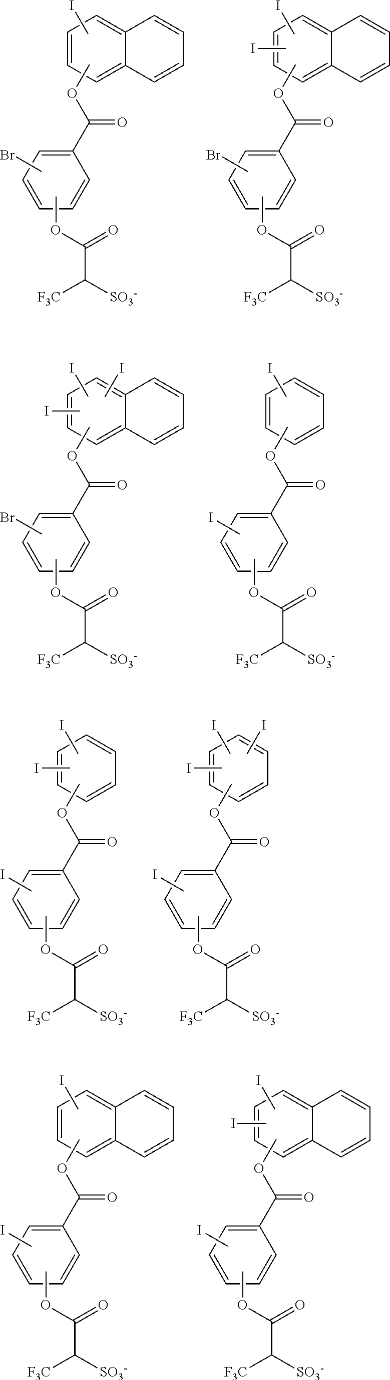

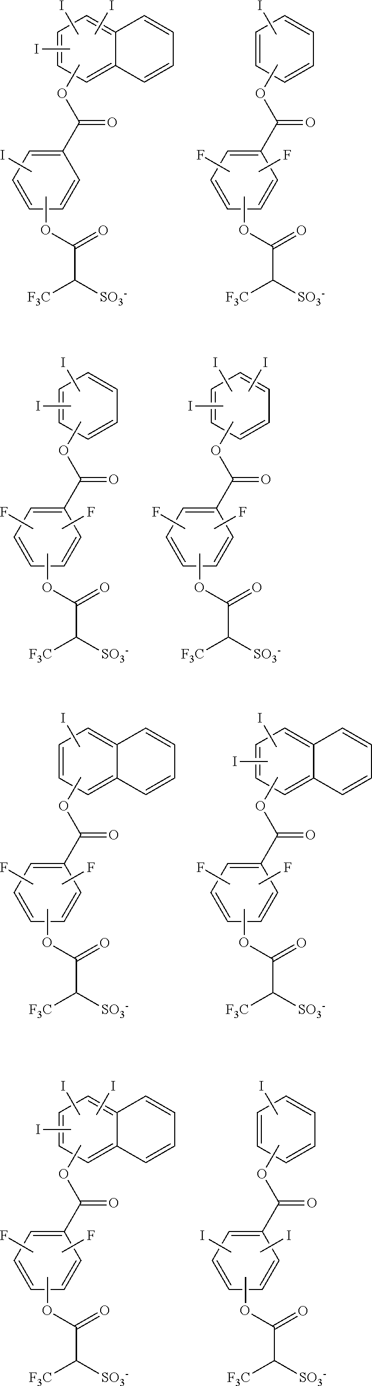

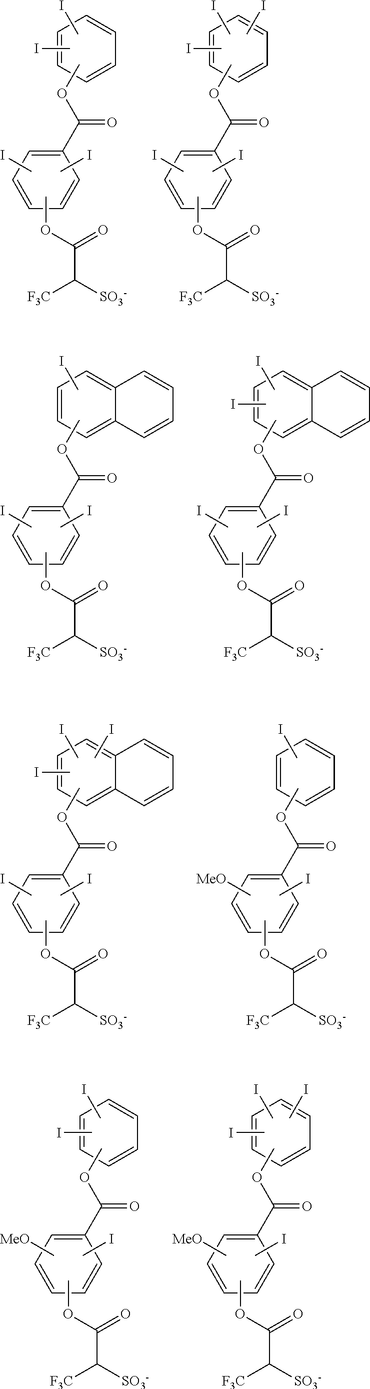

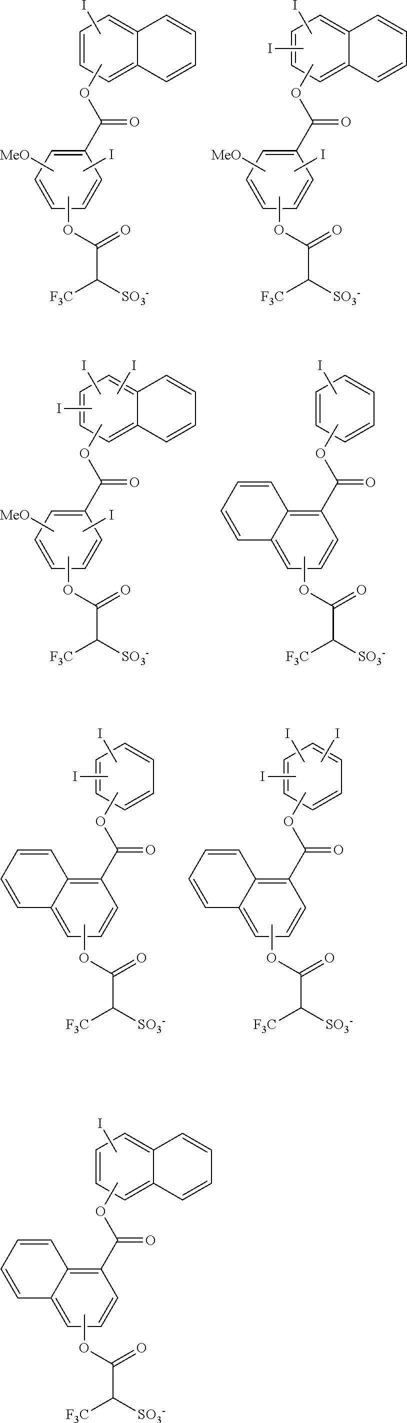

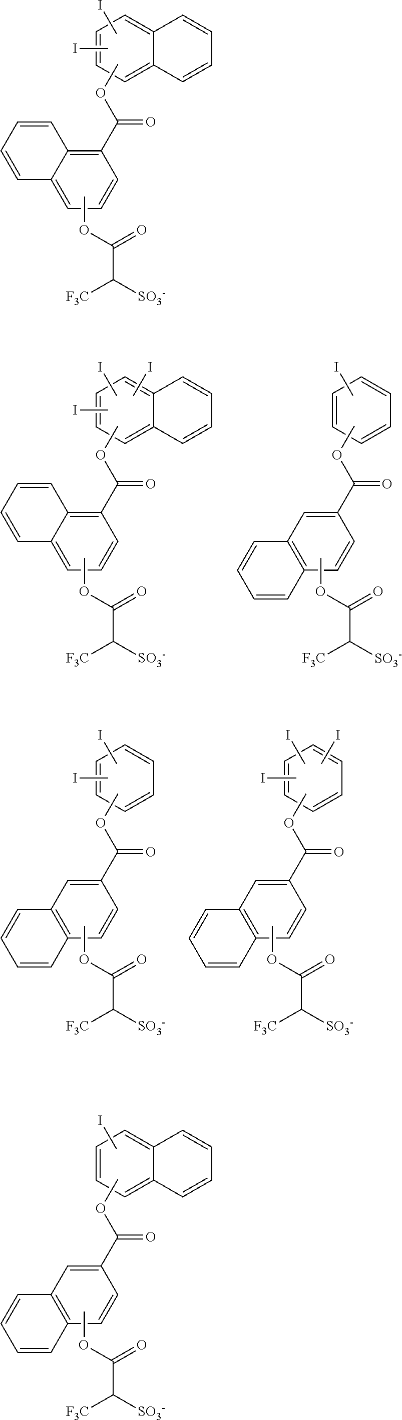

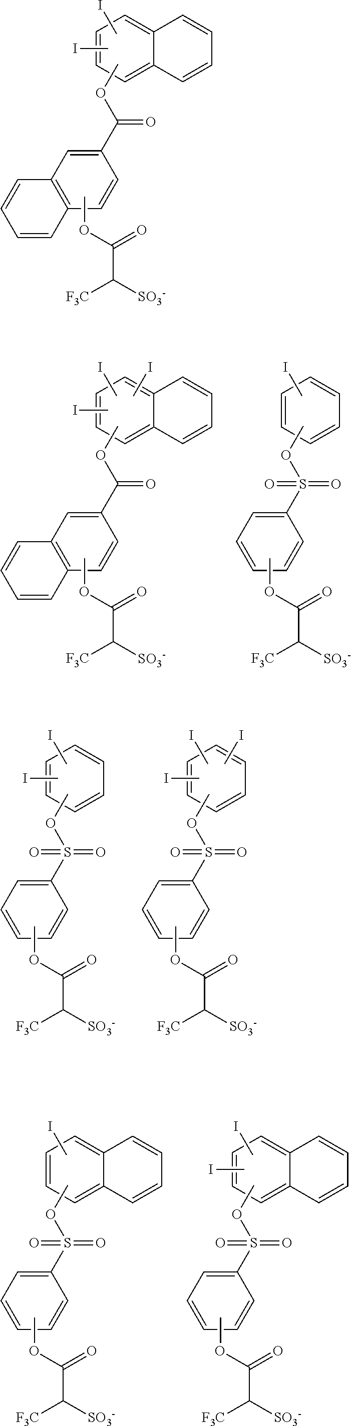

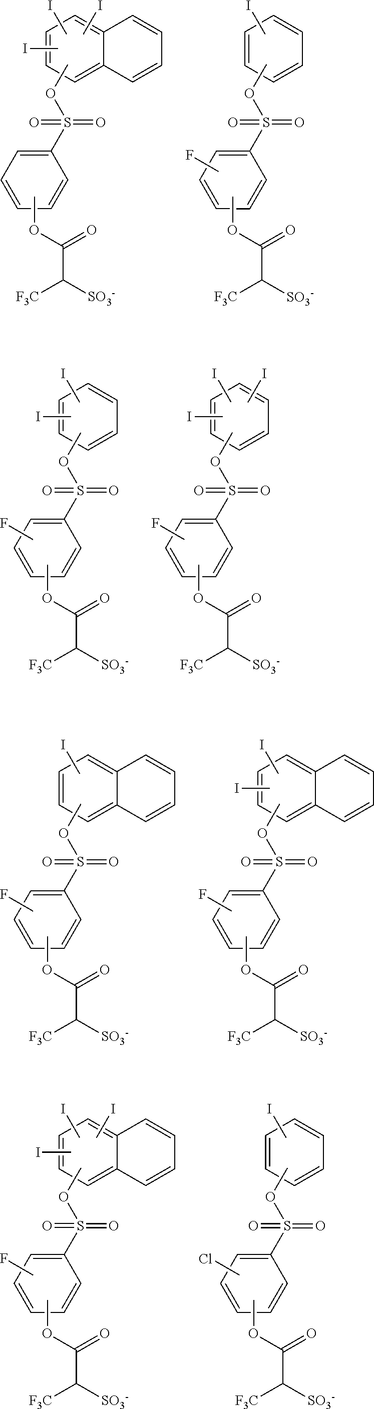

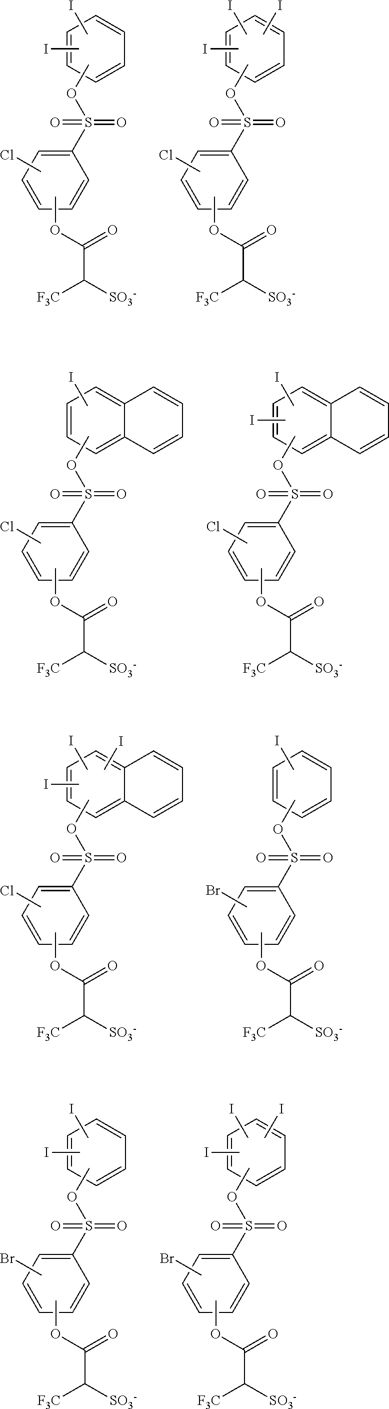

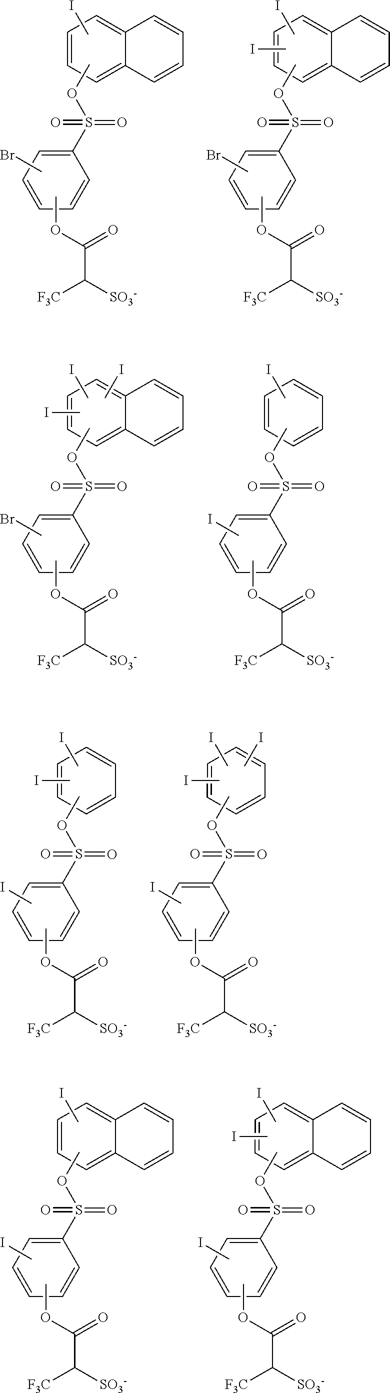

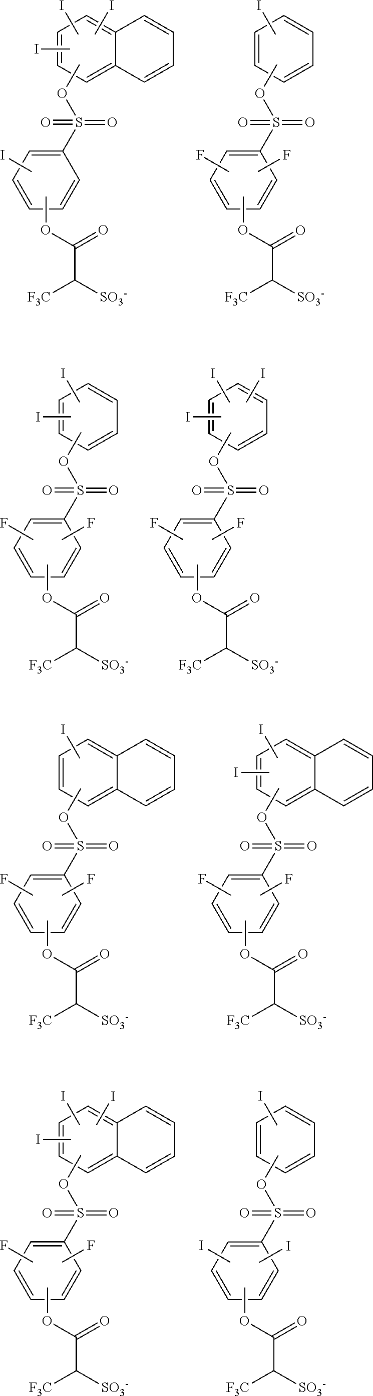

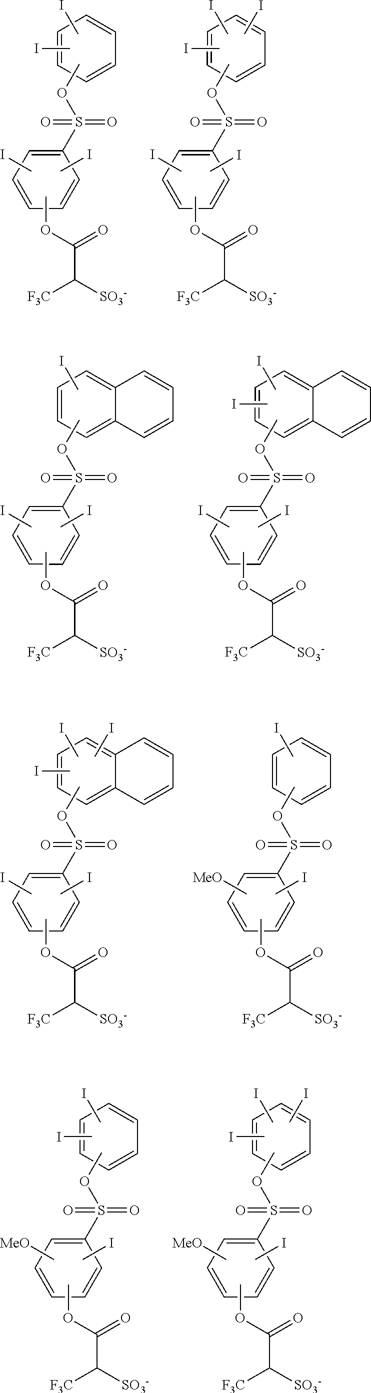

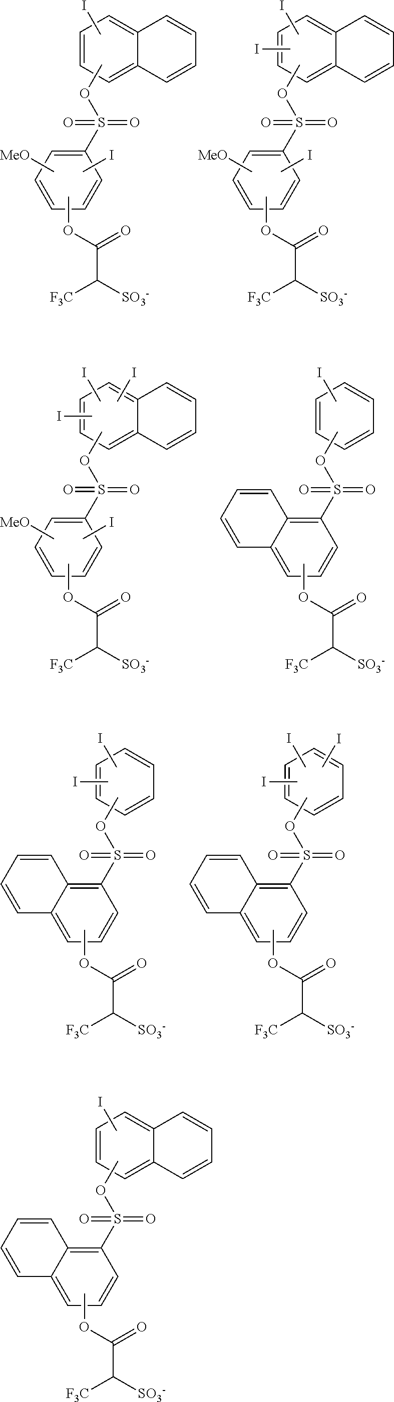

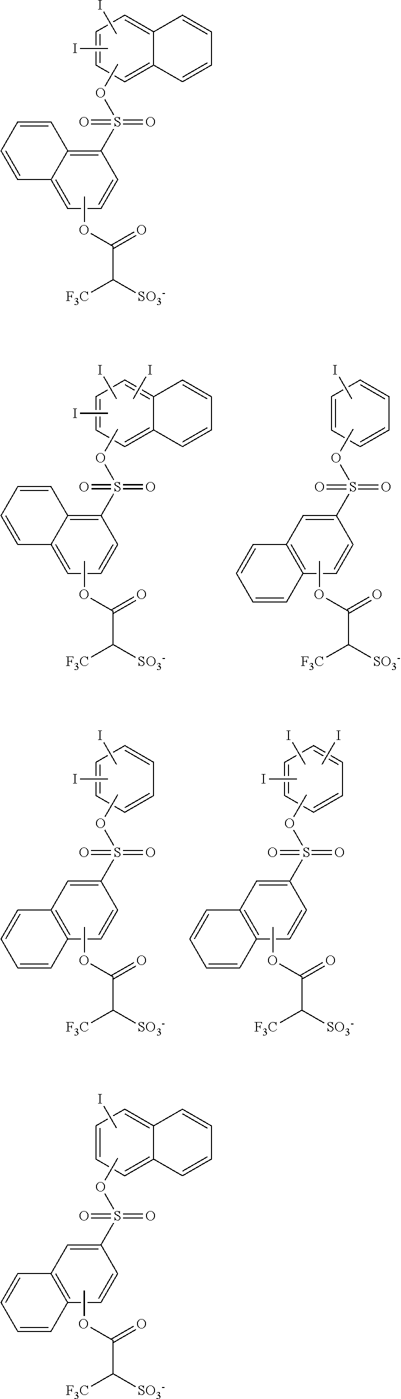

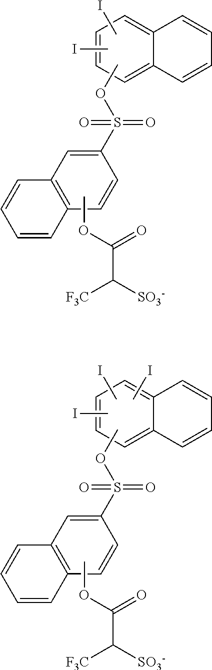

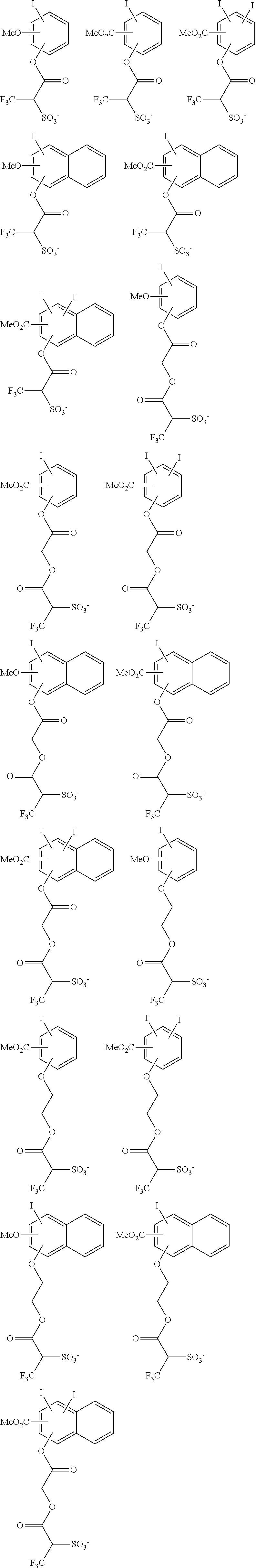

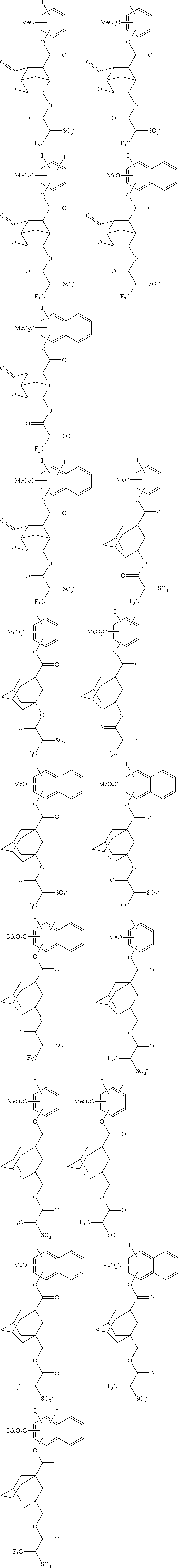

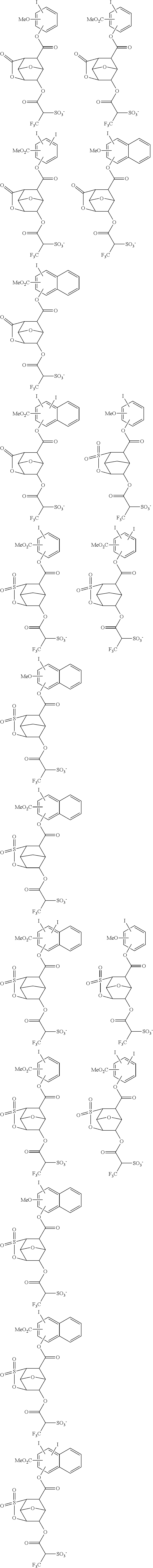

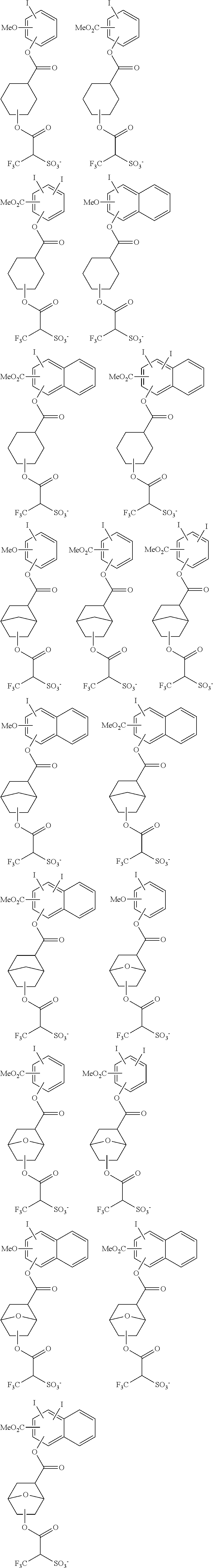

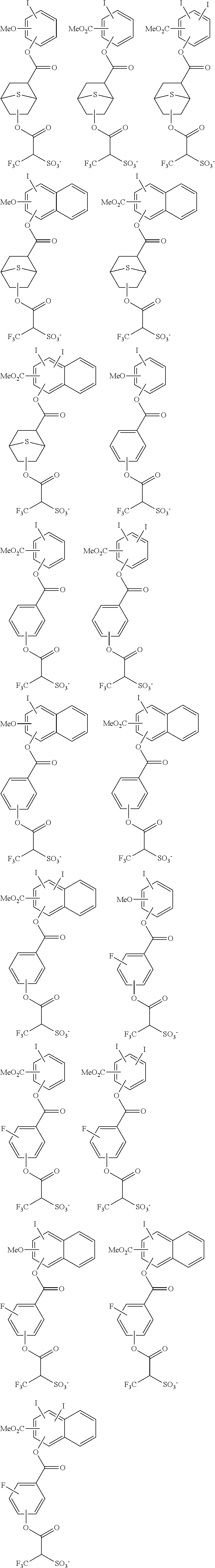

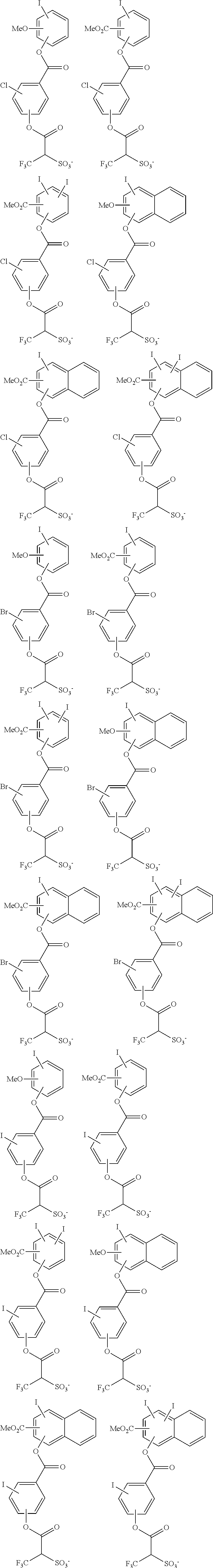

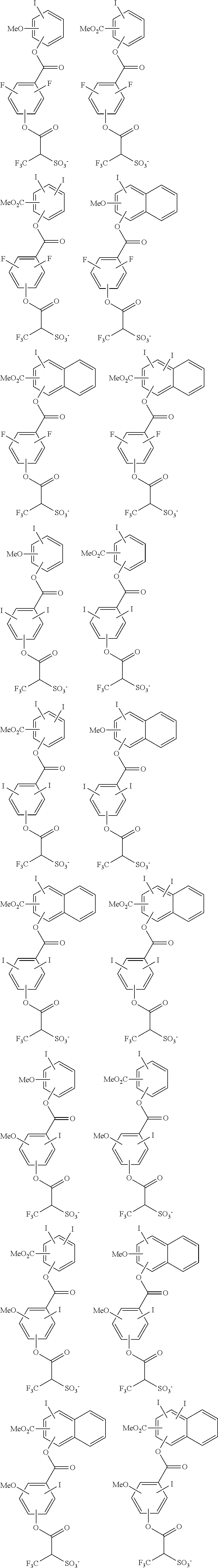

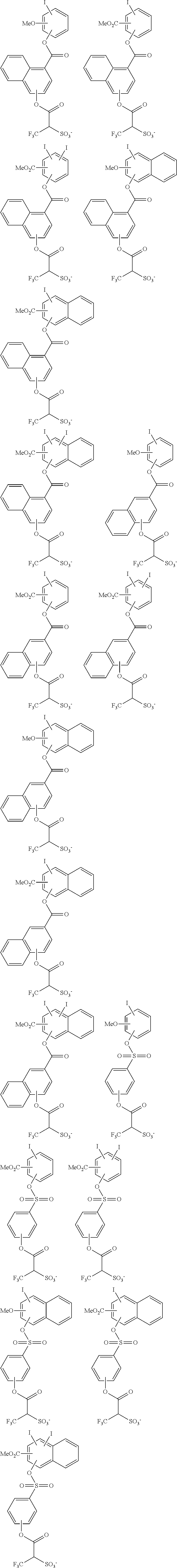

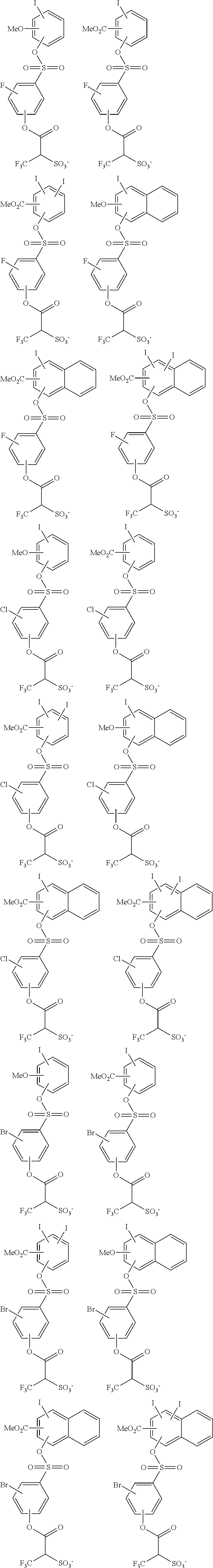

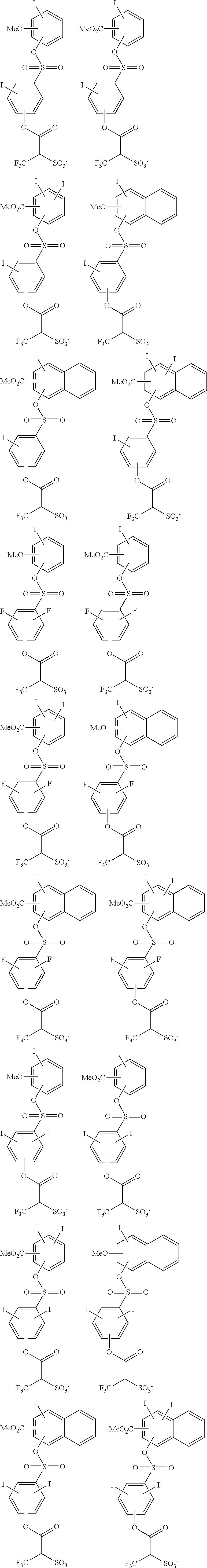

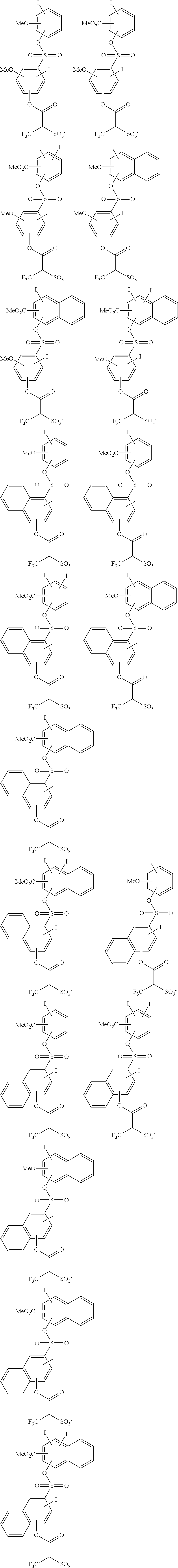

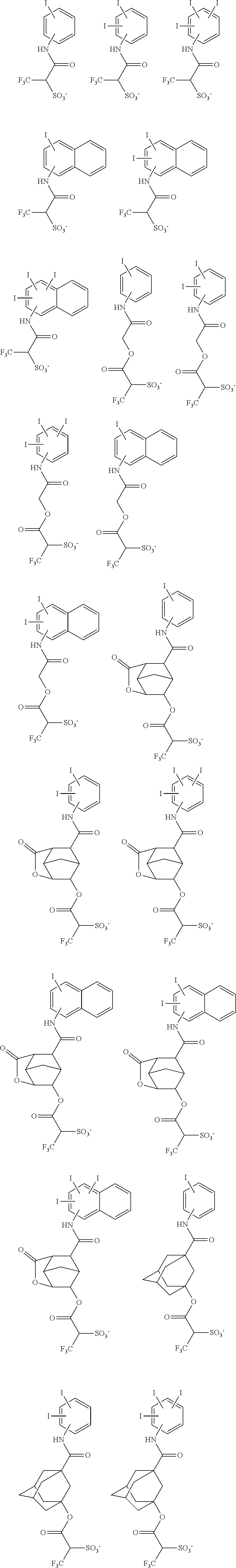

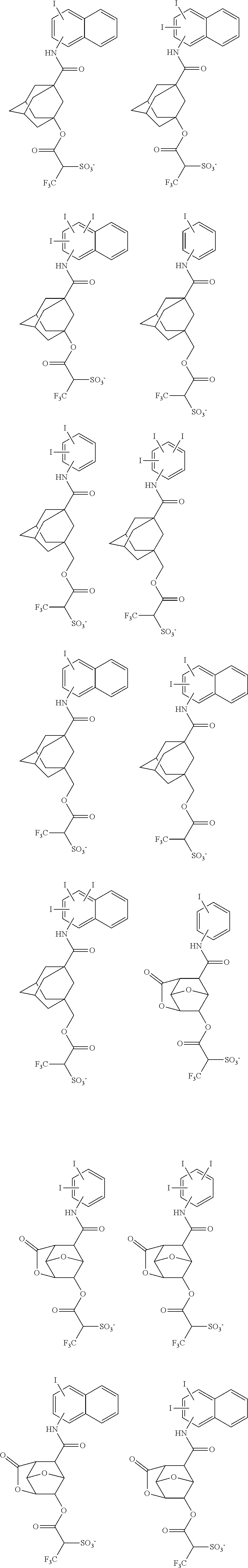

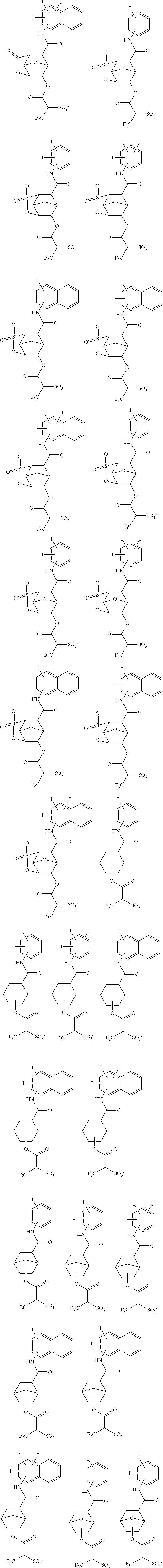

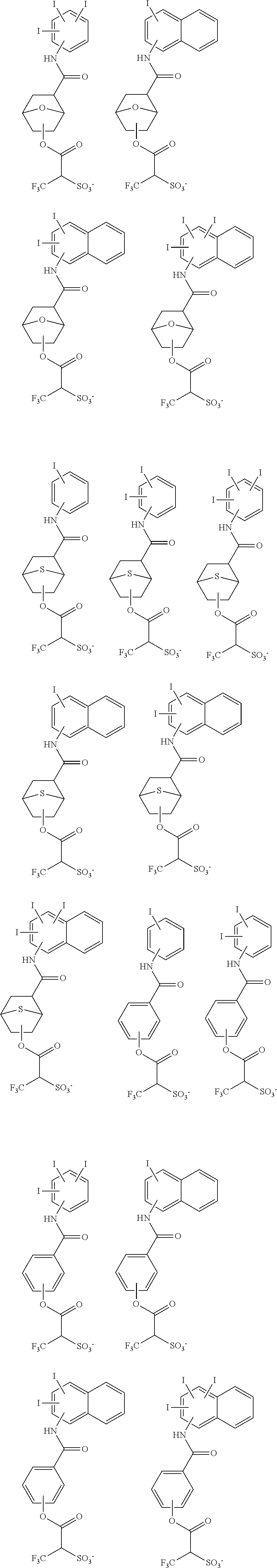

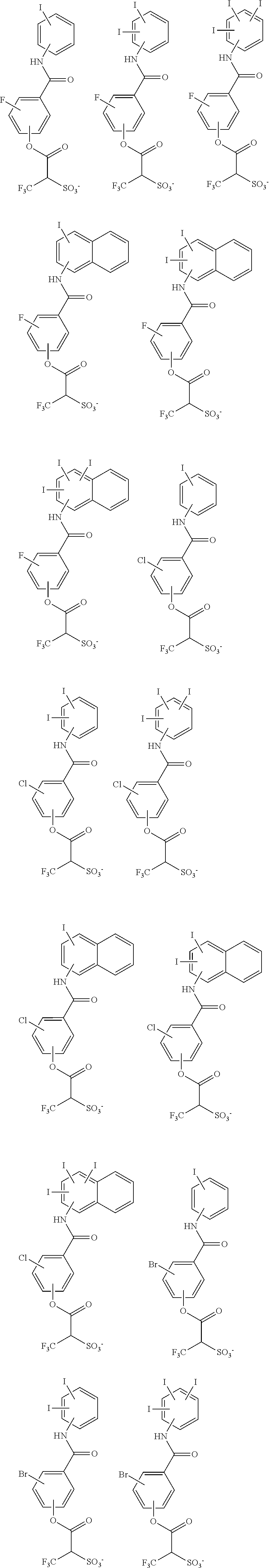

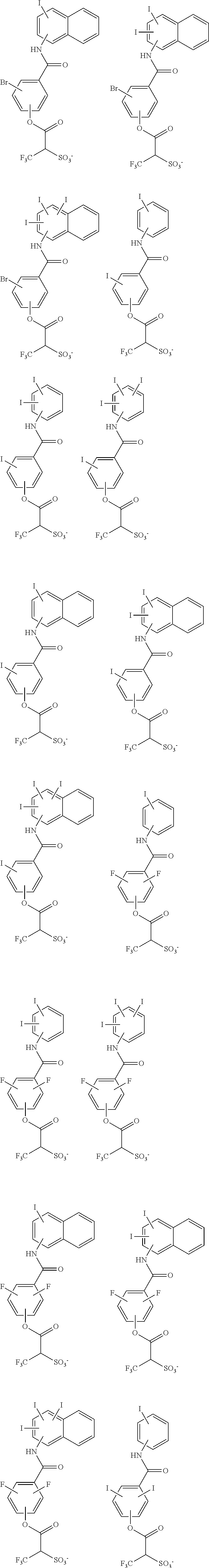

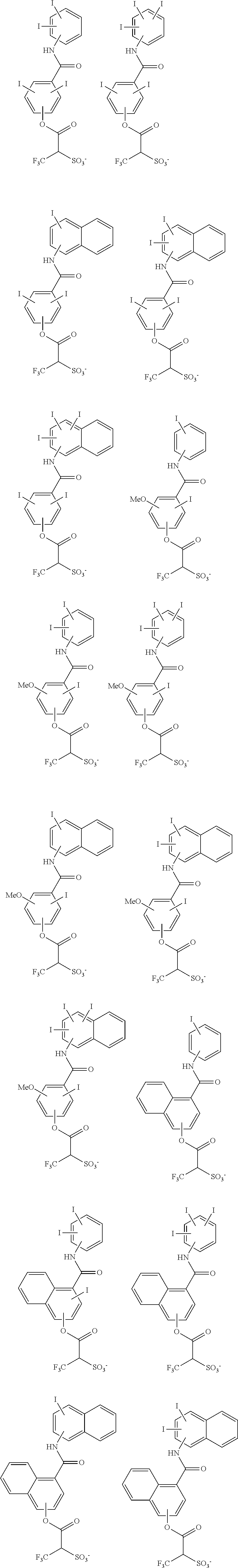

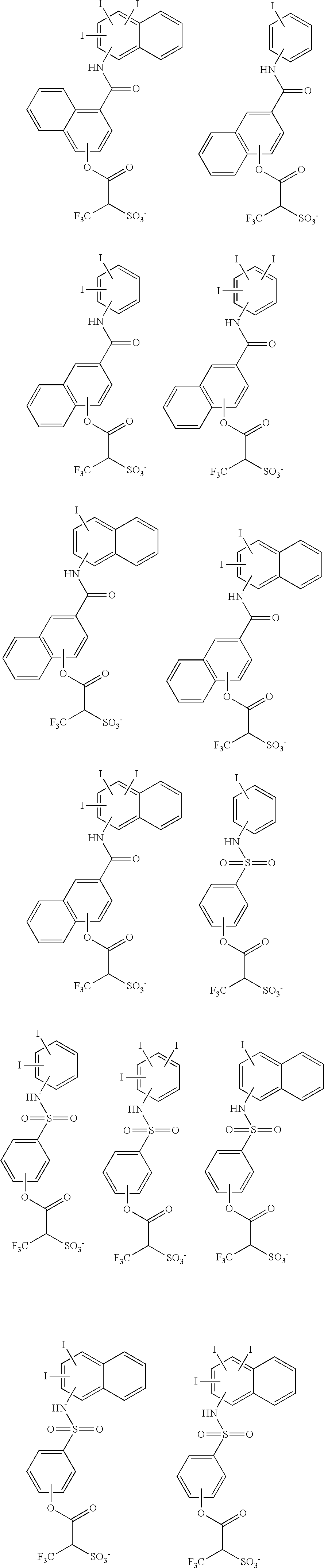

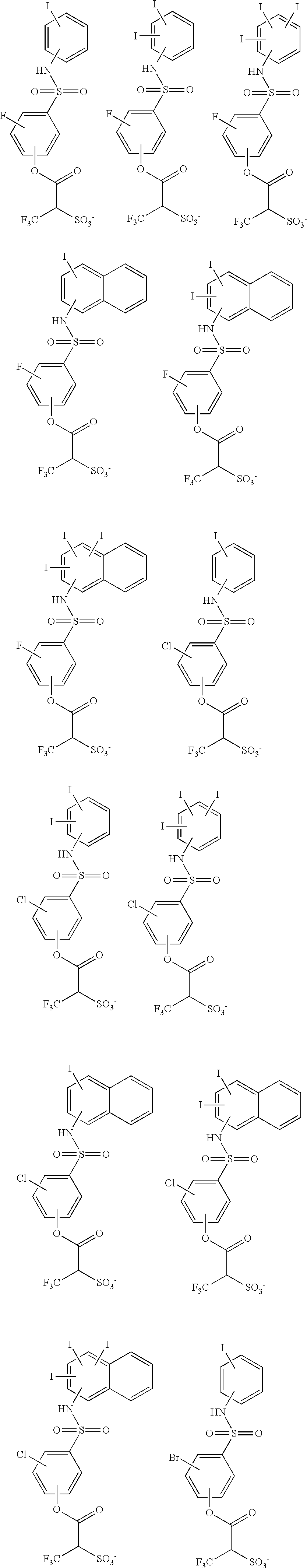

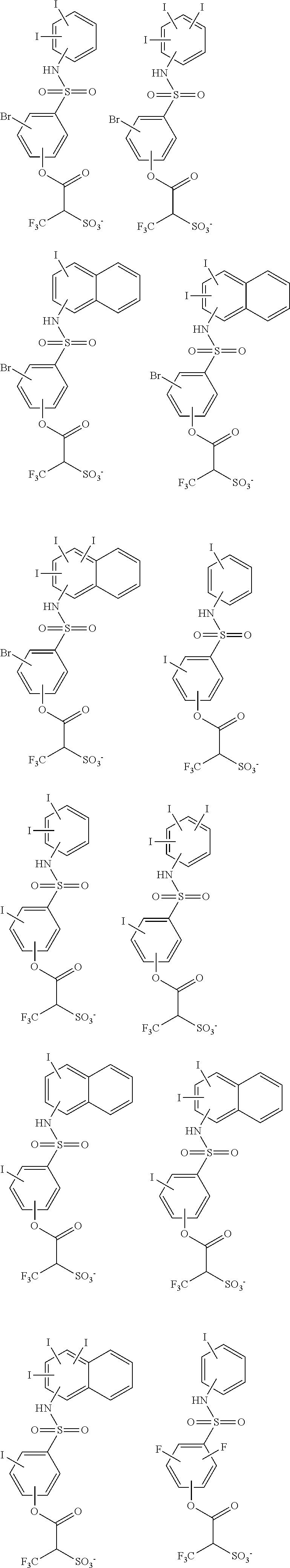

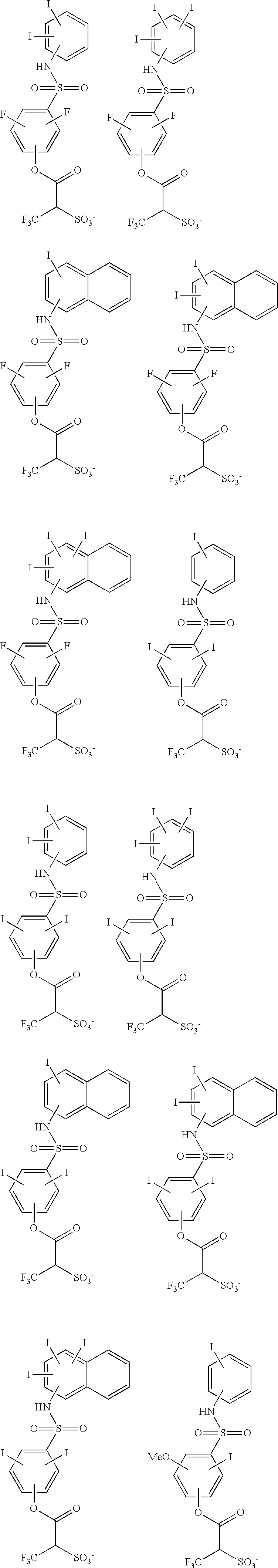

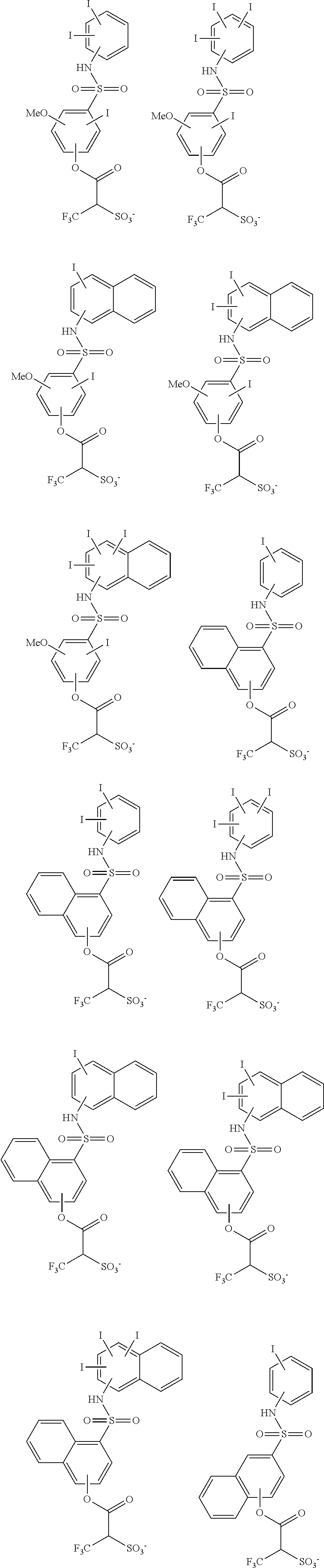

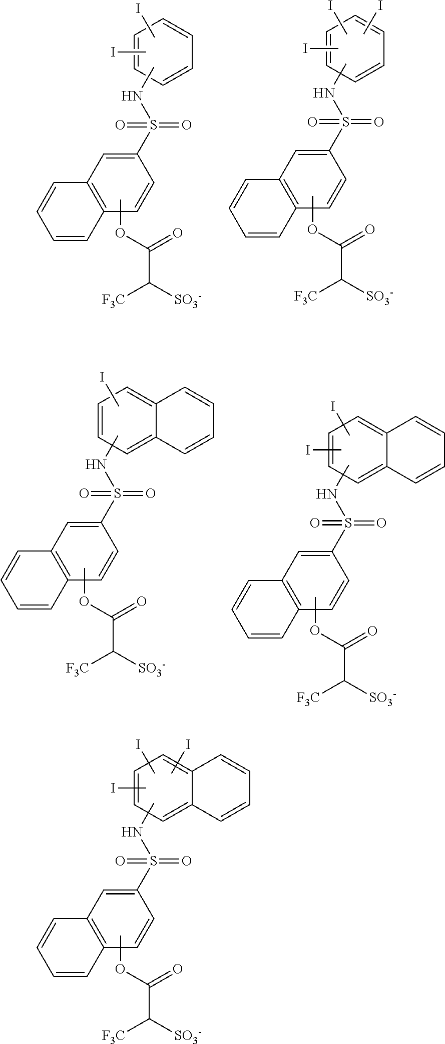

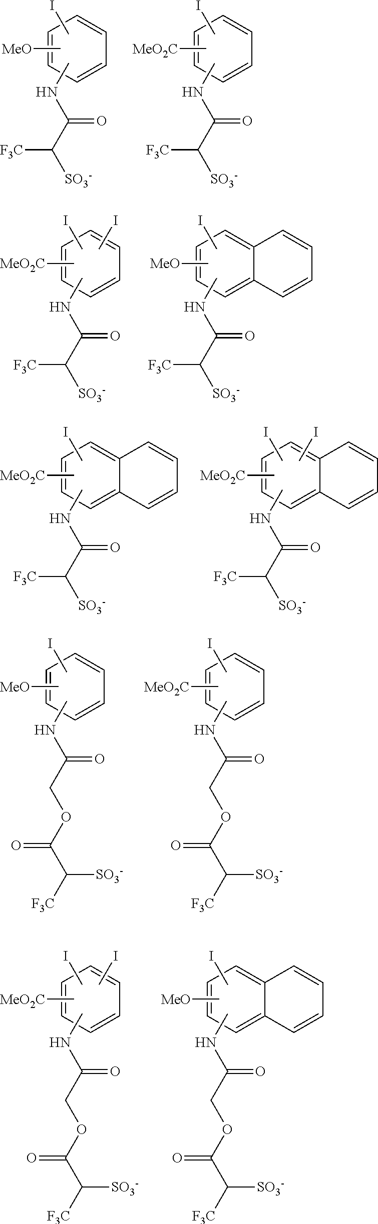

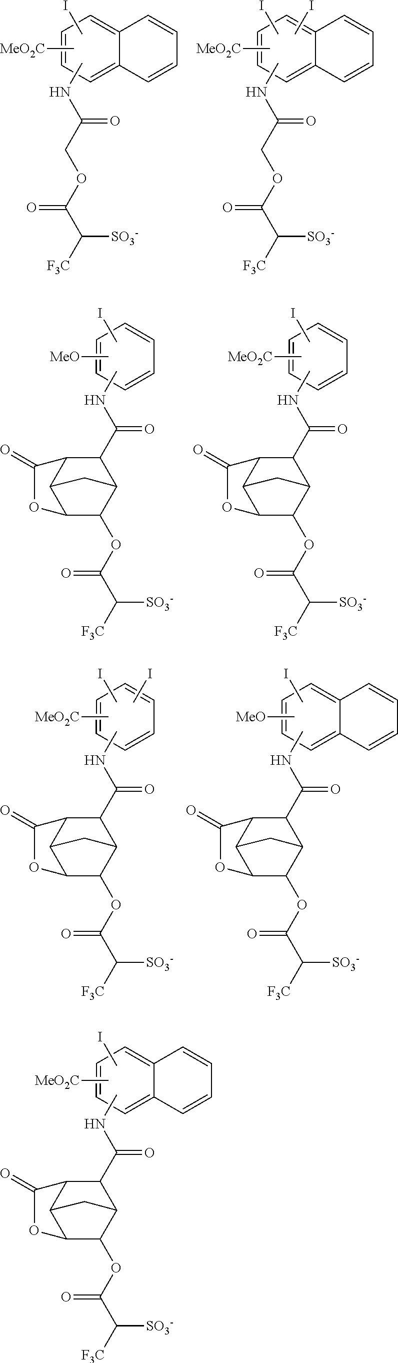

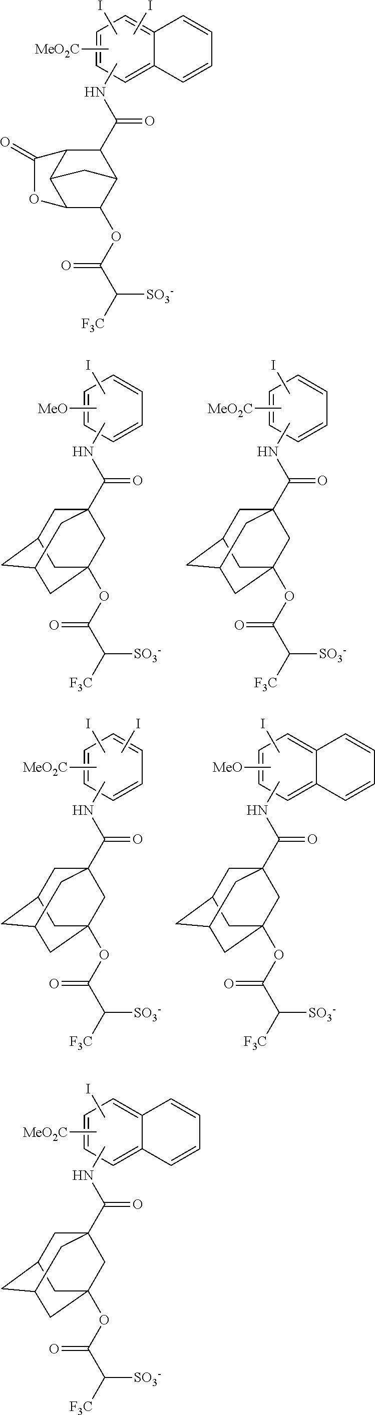

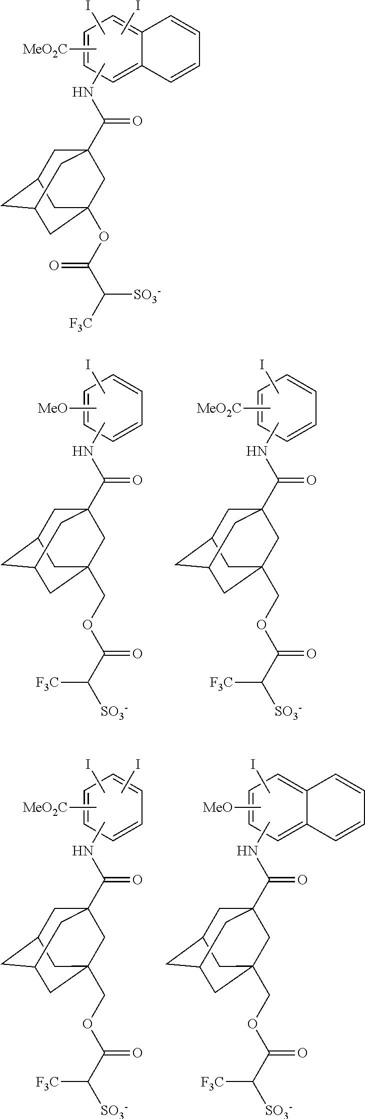

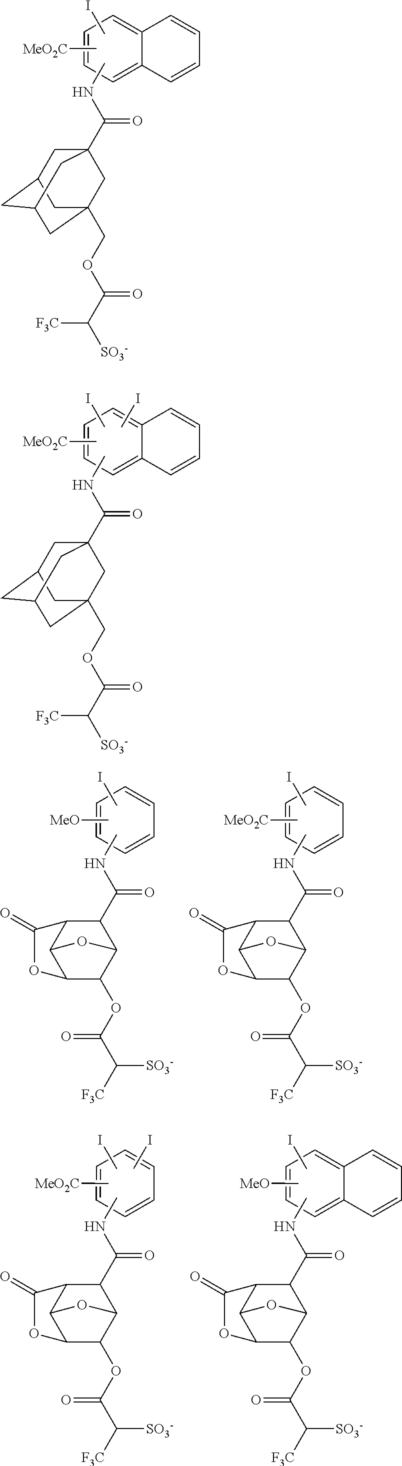

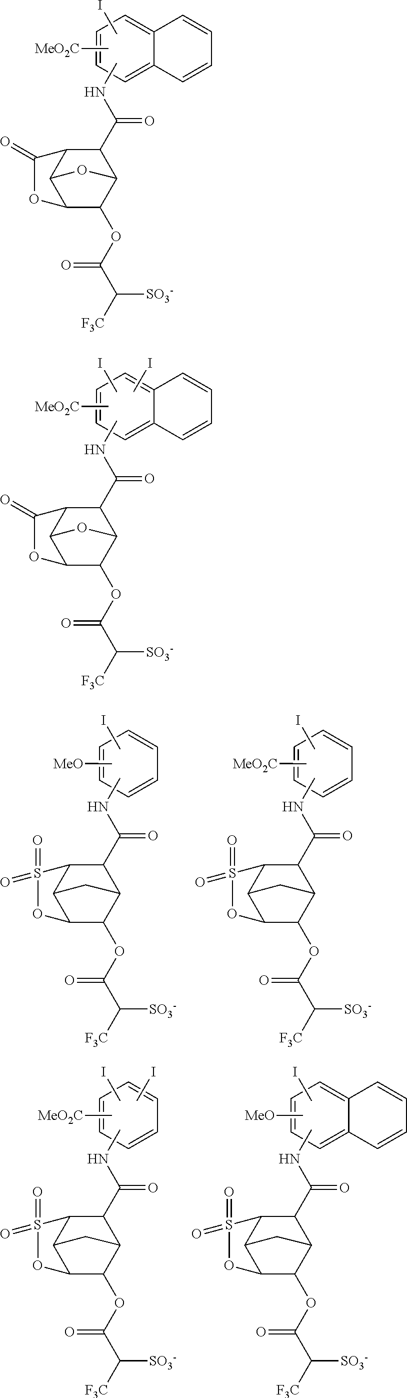

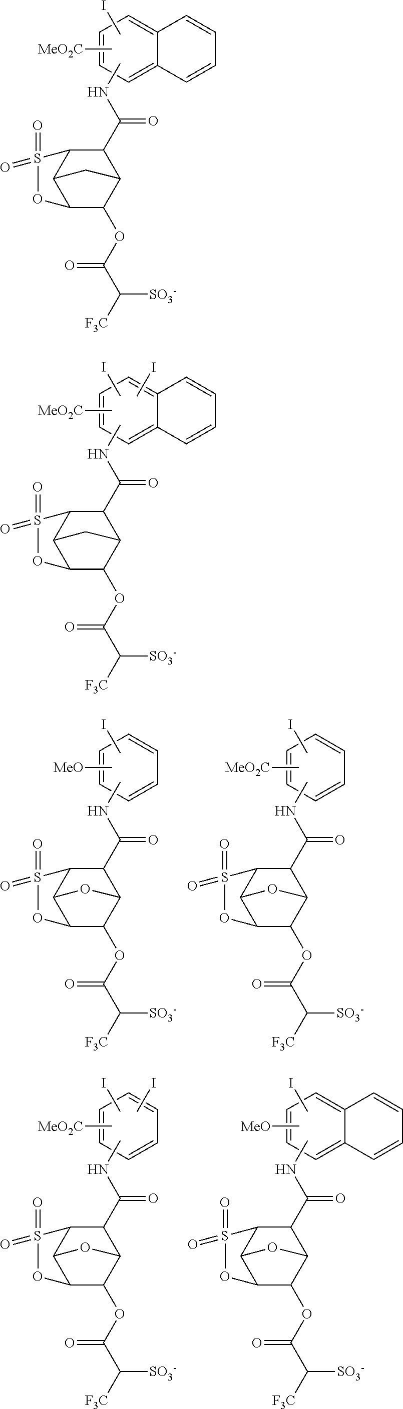

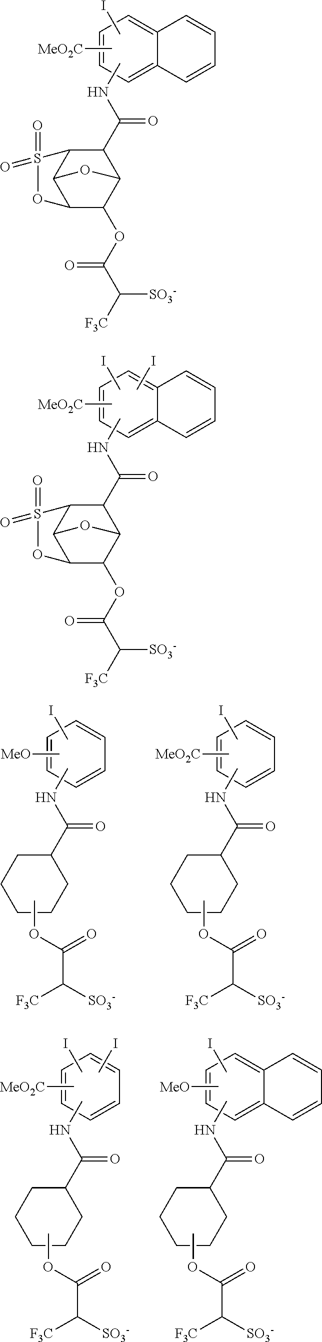

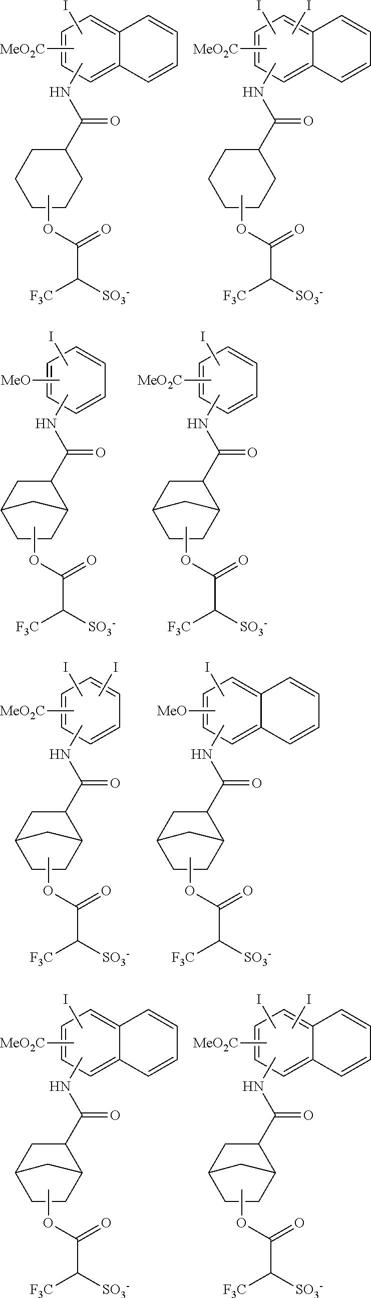

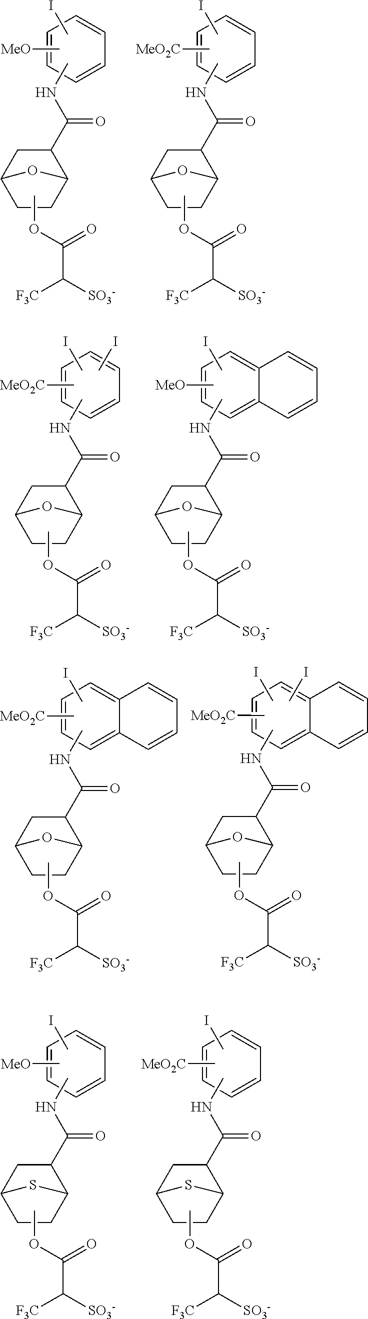

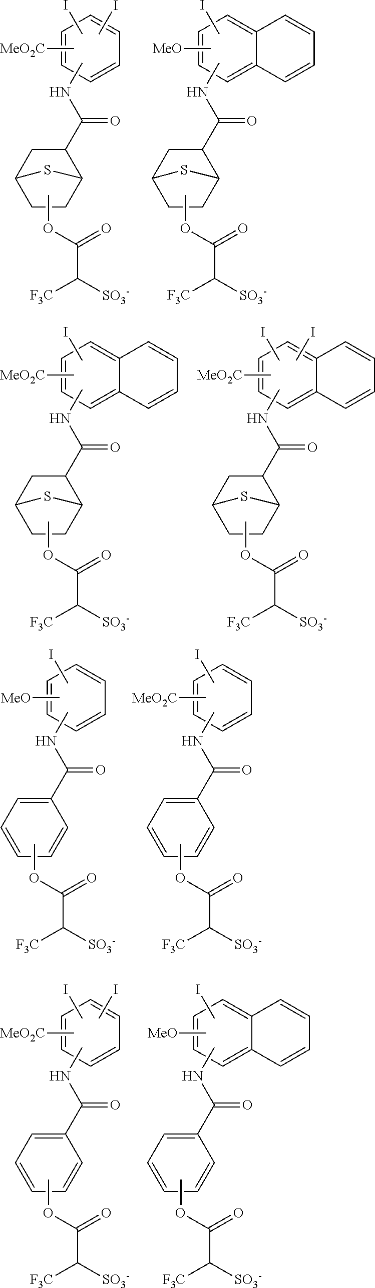

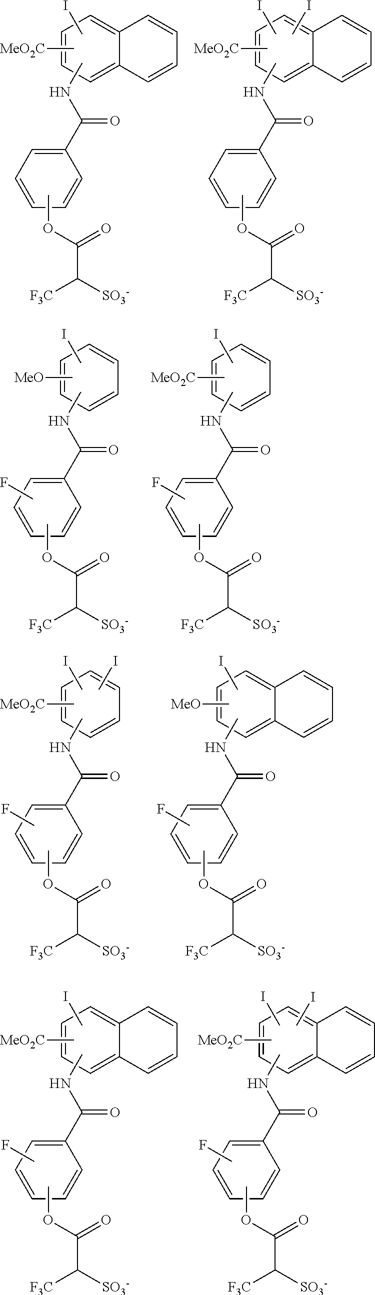

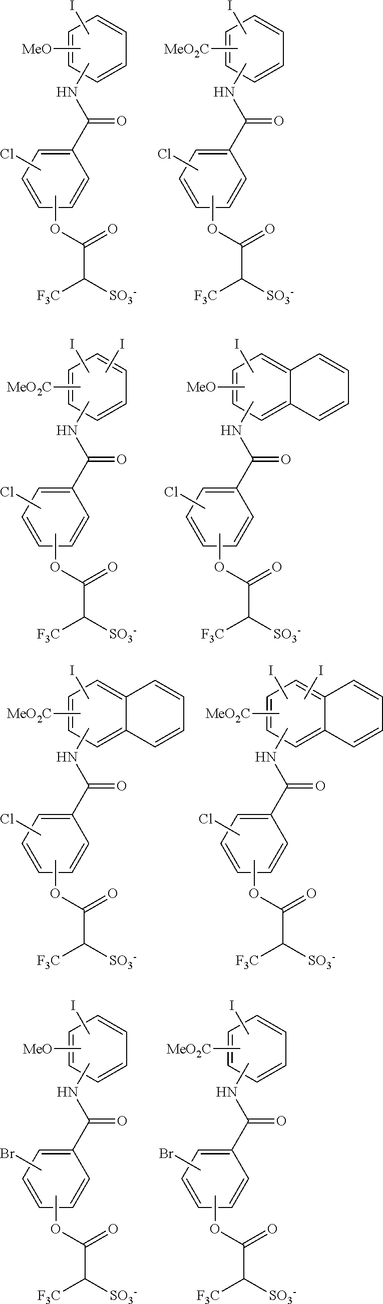

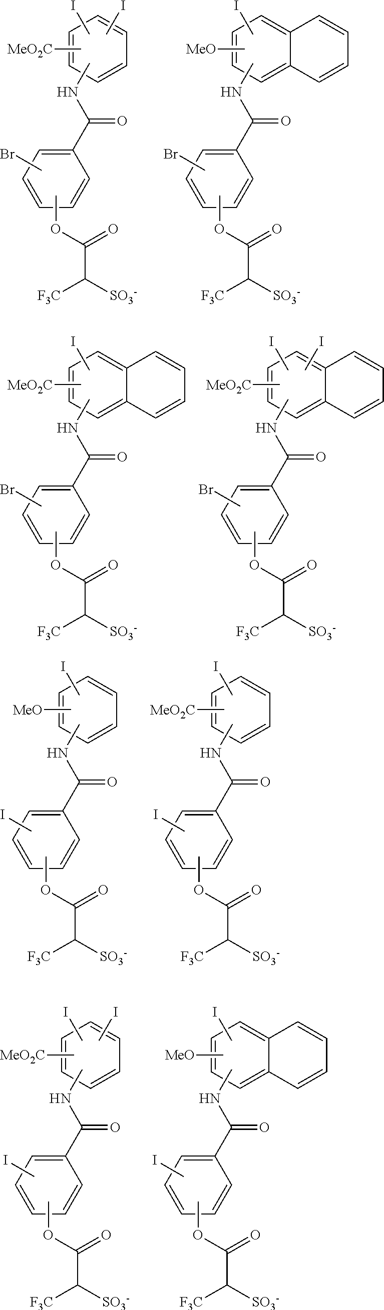

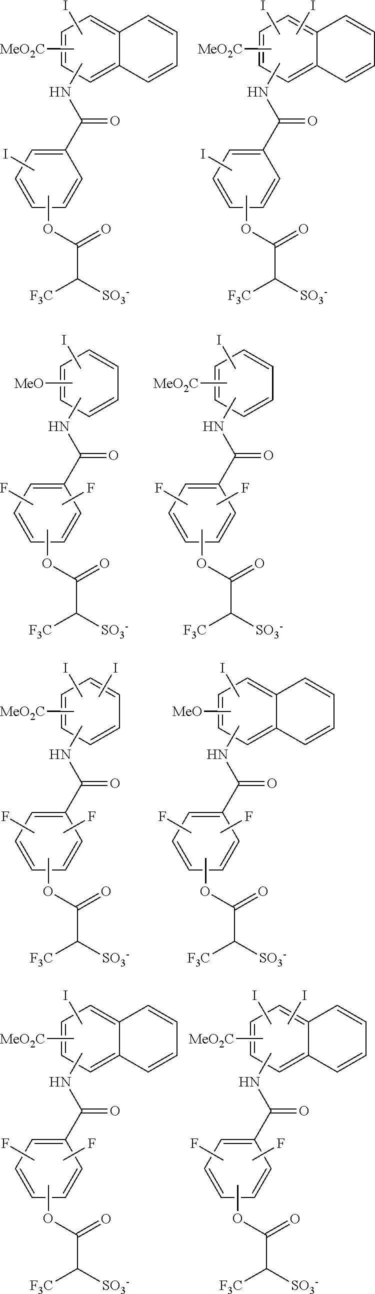

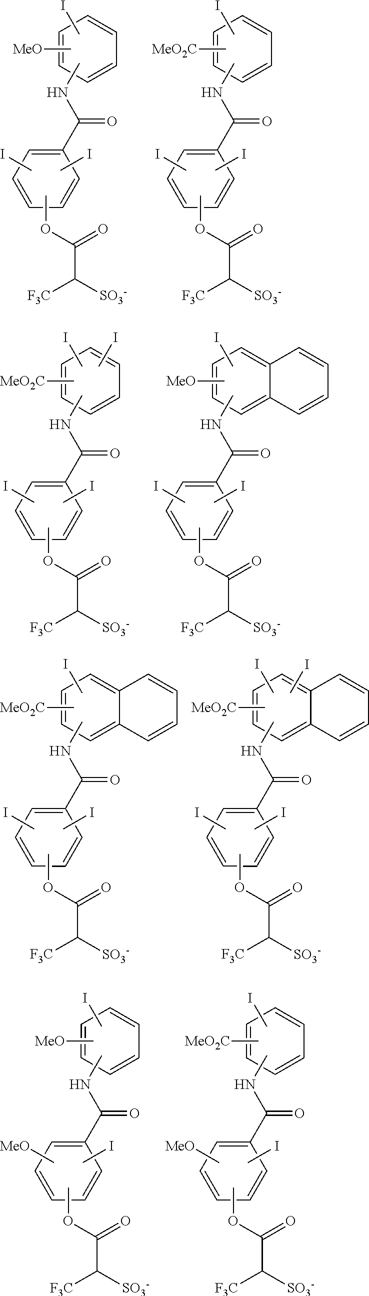

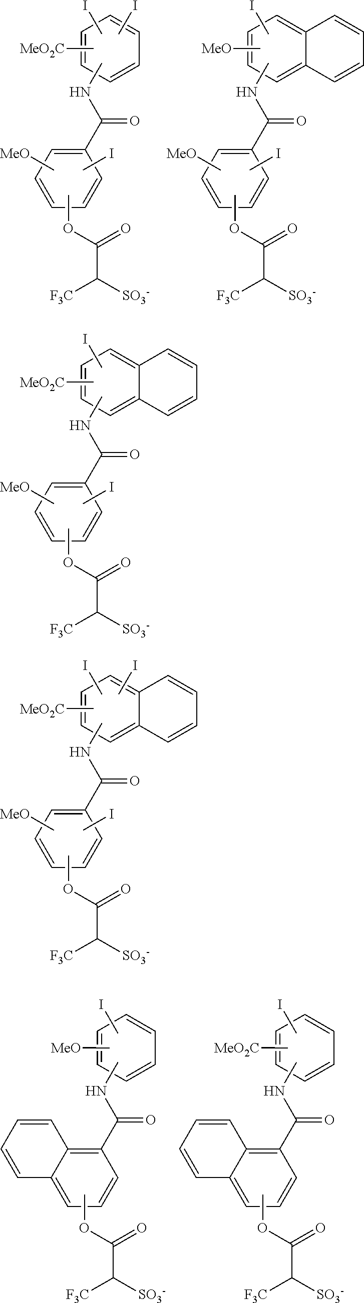

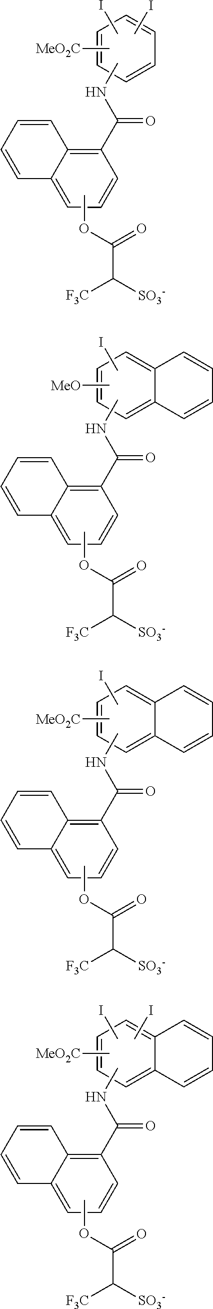

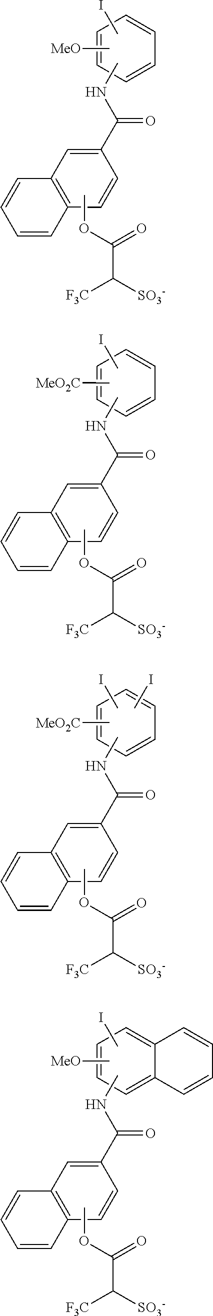

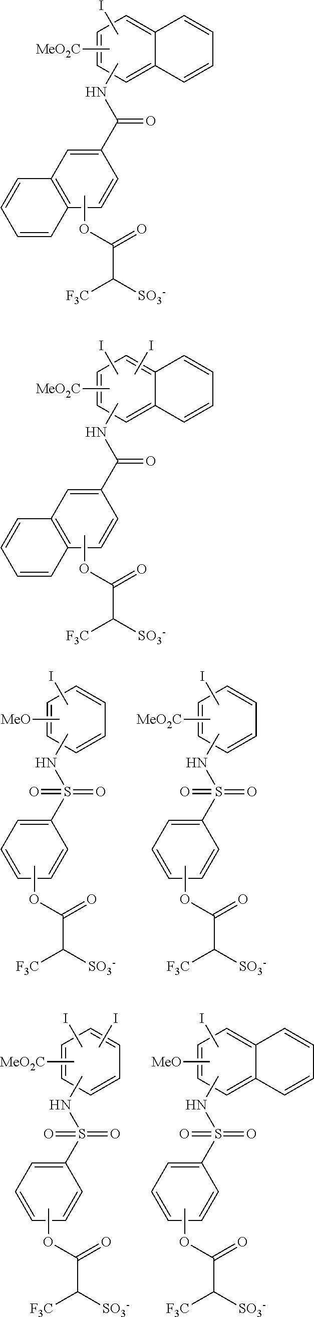

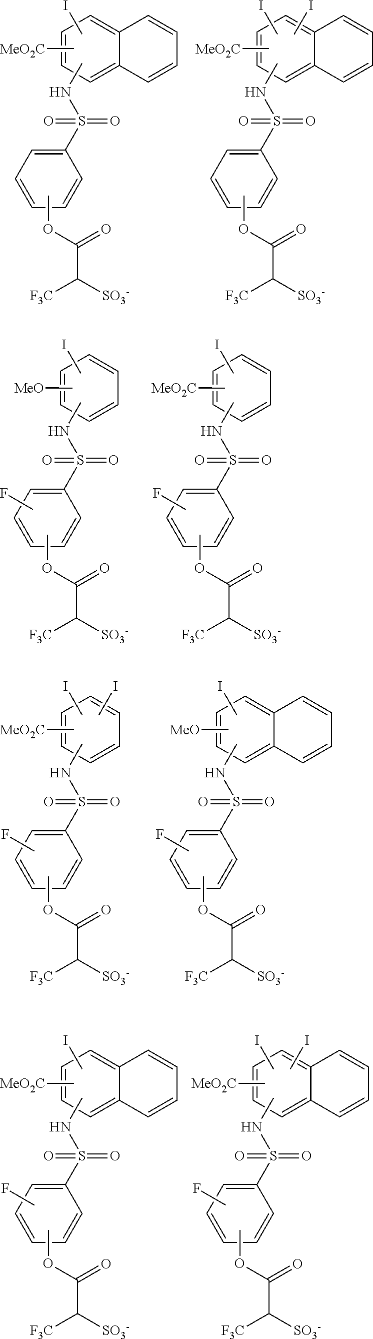

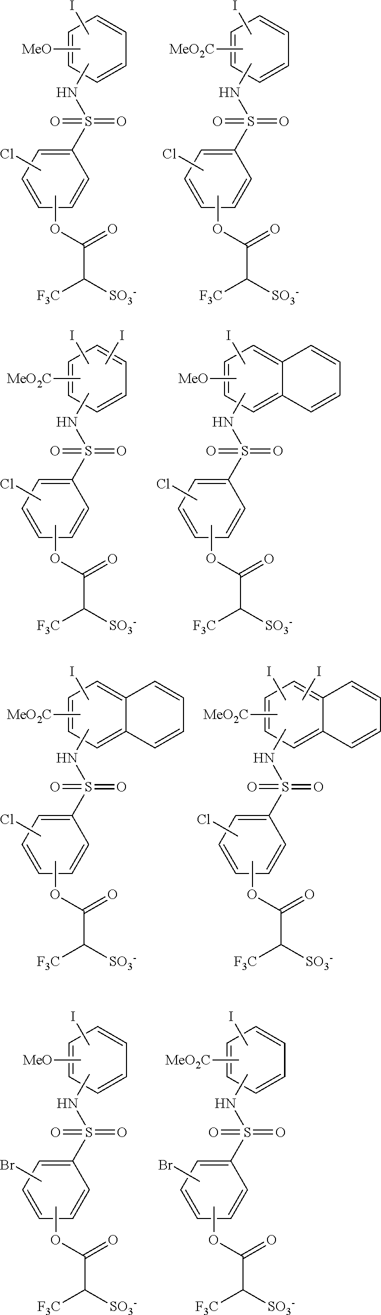

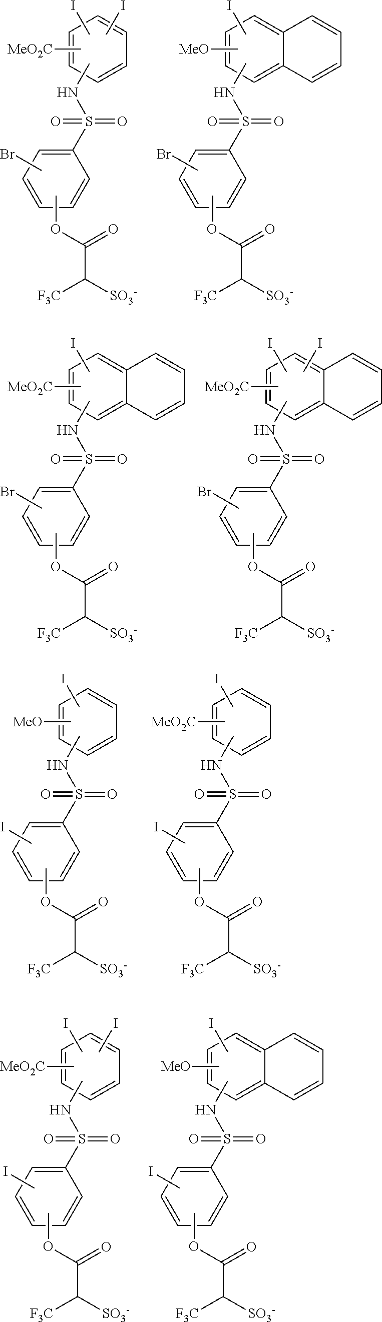

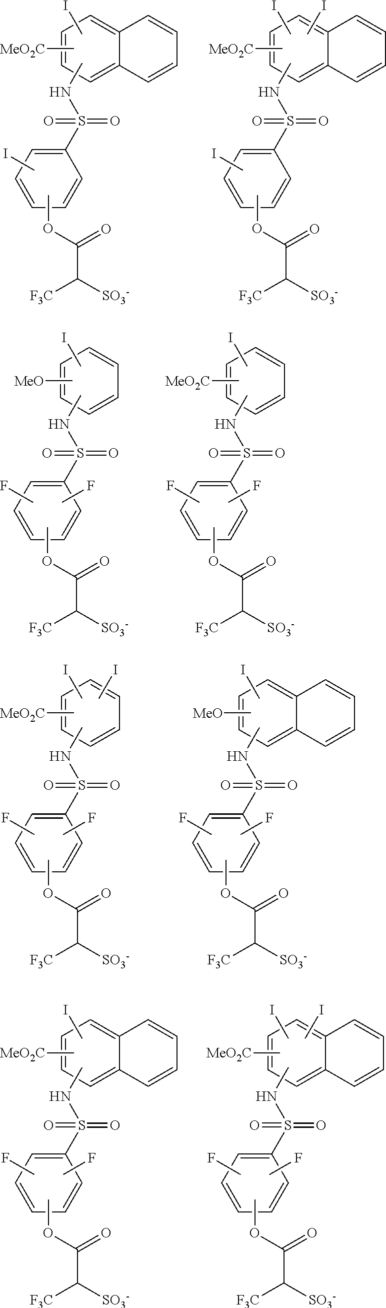

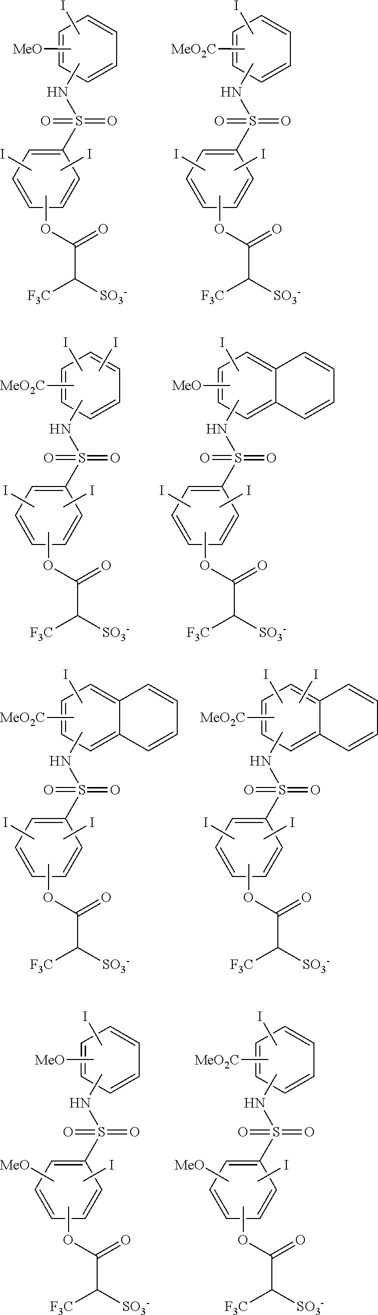

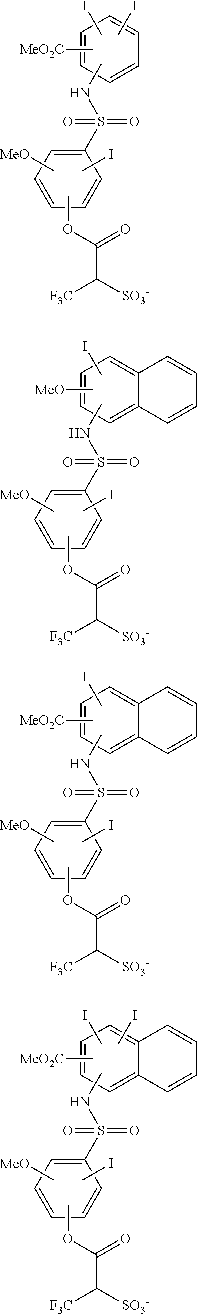

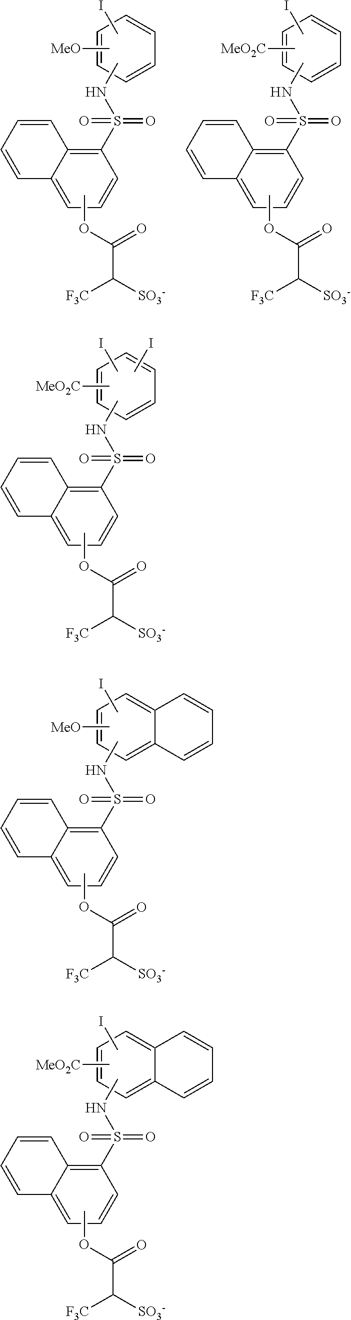

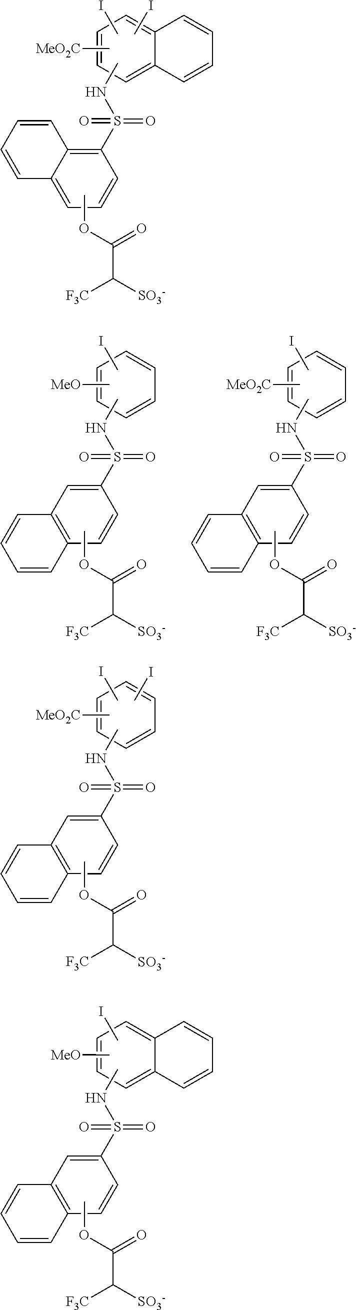

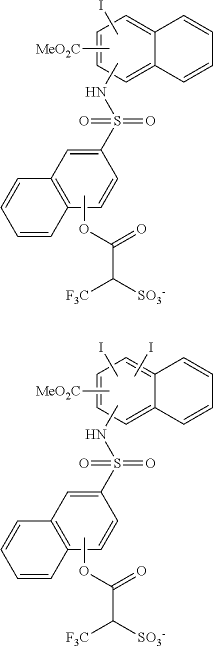

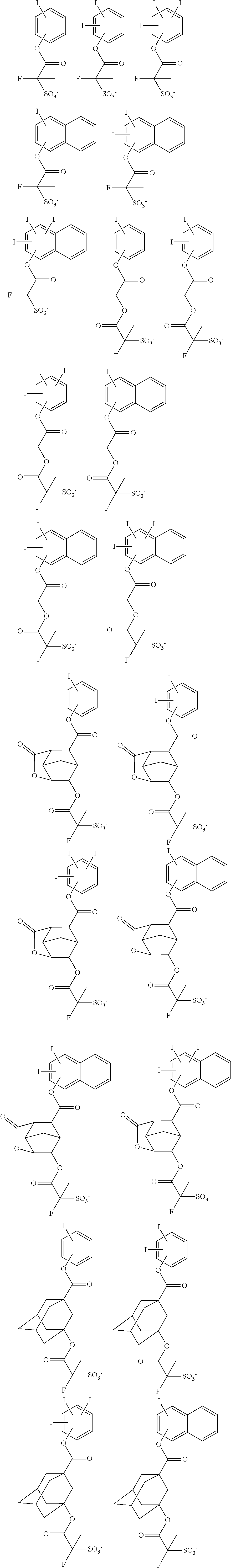

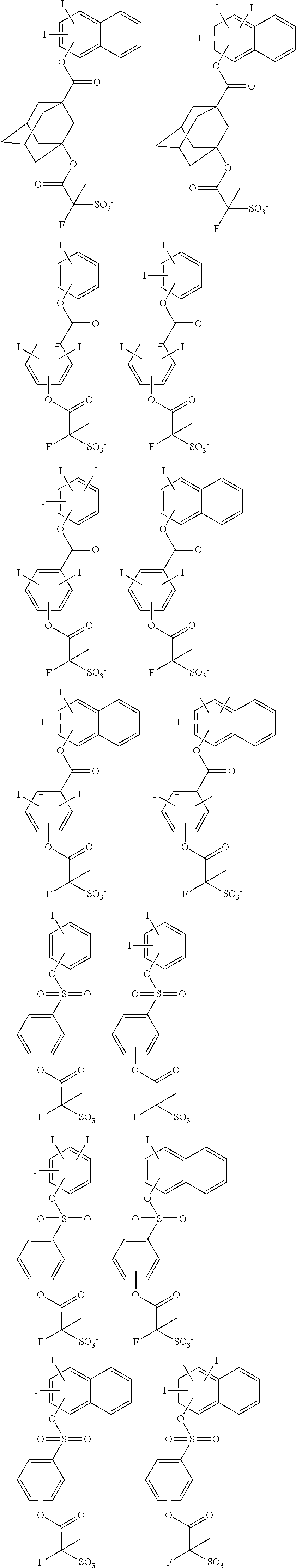

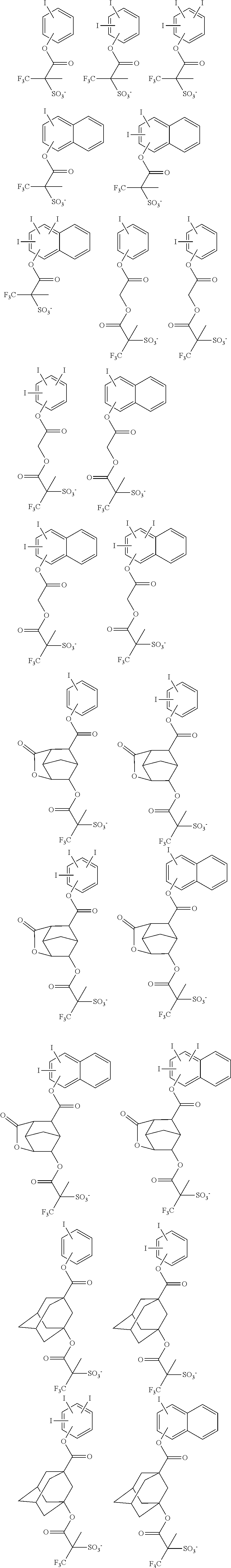

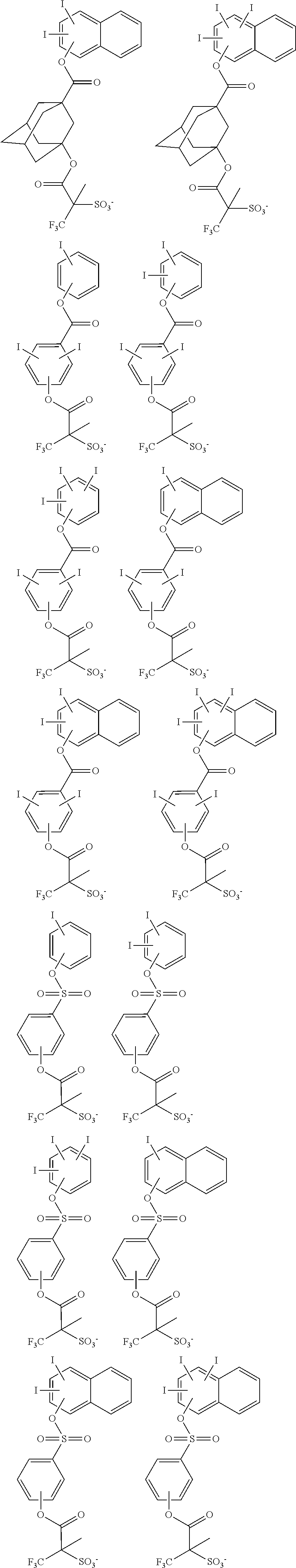

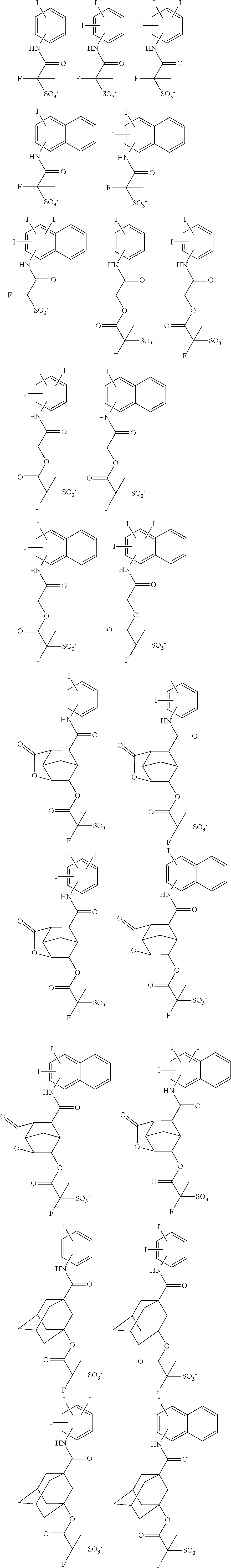

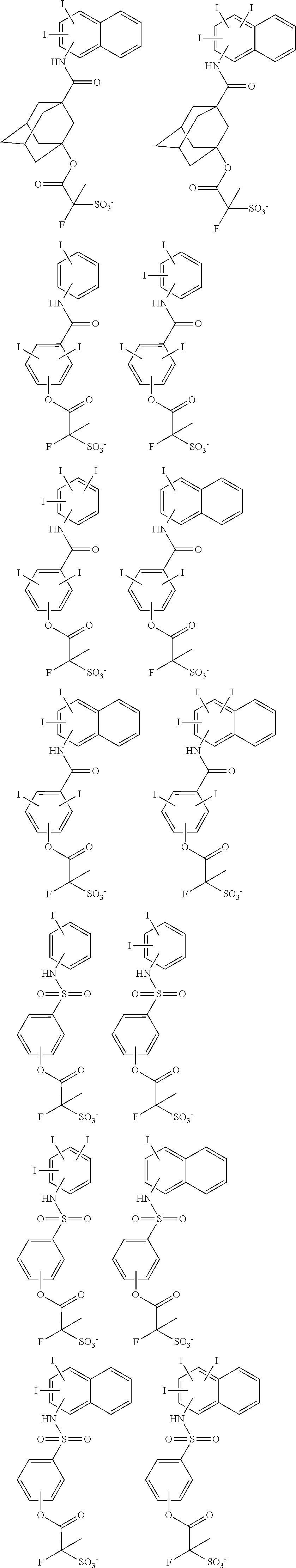

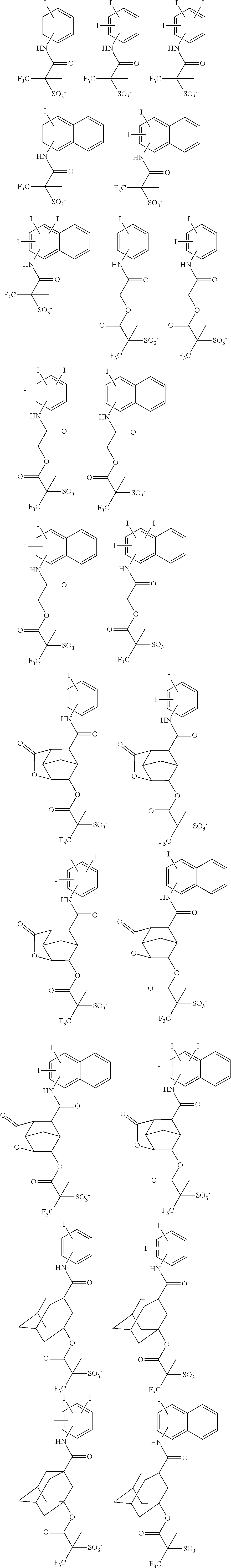

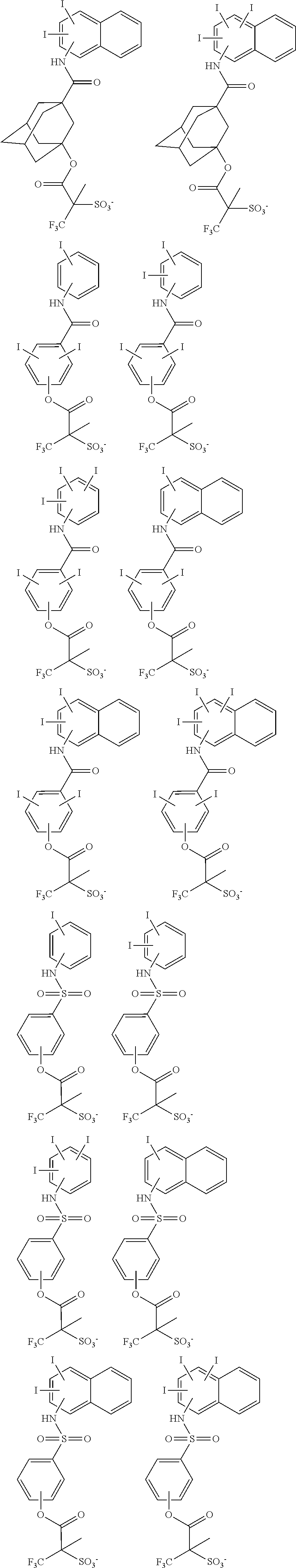

In the general formula (1), Z⁺ is an onium cation. Z⁺ is preferably a sulfonium cation represented by the following general formula (Z-1) or an iodonium cation represented by the following general formula (Z-2).

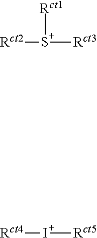

In the formula, R^{ct1} to R^{ct5} are each independently a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

In the general formulae (Z-1) and (Z-2), R^{ct1} to R^{ct5} are each independently a halogen atom, or a hydrocarbyl group having 1 to 30 carbon atoms and optionally containing a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, a 1-propenyl group, a 2-propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; and groups obtained by combining these groups, and aryl groups are preferred. In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In addition, R^{ct1} and R^{ct2} may be bonded to each other to form a ring together with the sulfur atom to which they are bonded. In this case, specific examples of the ring structure include those represented by the following formulae.

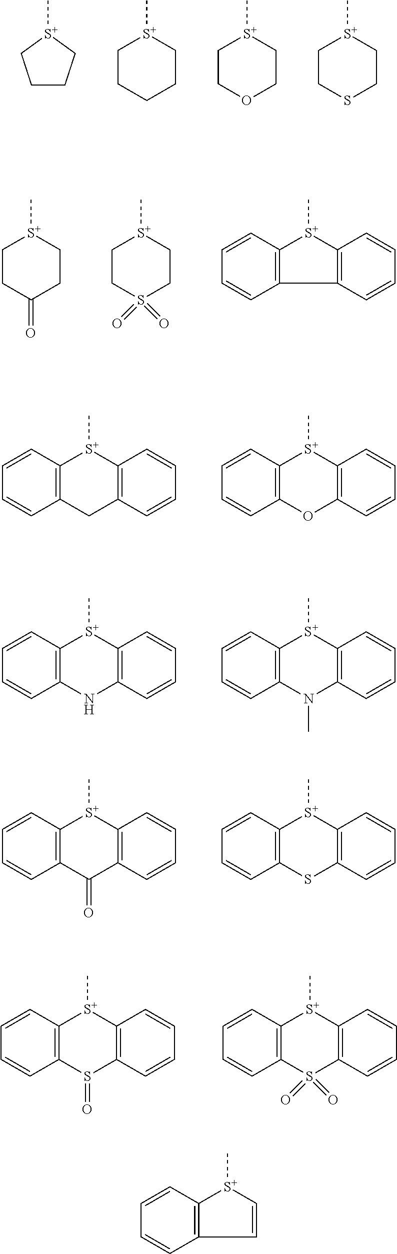

In the formulae, the dashed line indicates the bond to R^{ct3}.

Specific examples of the sulfonium cation represented by the general formula (Z-1) include those described in paragraphs to of JP2024-003744A, those described in paragraphs to of WO2024/128017A1, and those described in paragraphs to of JP7491173B2, but are not limited thereto.

Specific examples of the iodonium cation represented by the general formula (Z-2) include those described in paragraph of JP2024-000259A, but are not limited thereto.

Z⁺ is also preferably a sulfonium cation represented by the following general formula (Z-3).

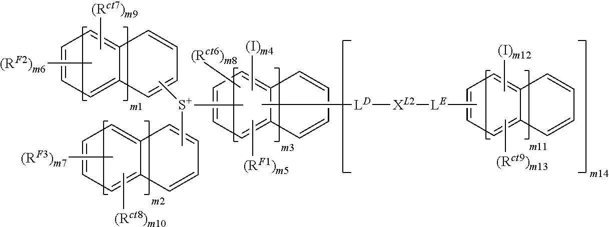

In the formula, “m1” is 0 or 1; “m2” is 0 or 1; “m3” is 0 or 1; “m4” is an integer from 0 to 4; “m5” is an integer from 0 to 4; “m6” is an integer from 0 to 6; “m7” is an integer from 0 to 6; “m8” is 0, 1 or 2; “m9” is 0, 1 or 2; “m10” is 0, 1 or 2; “m11” is 0 or 1; “m12” is an integer from 0 to 4; “m13” is 0, 1 or 2; “m14” is 0, 1 or 2; provided that when “m1” is 0, 0≤m6+m9≤4, and when “m1” is 1, 0≤m6+m9≤6; when “m2” is 0, 0≤m7+m10≤4, and when “m2” is 1, 0≤m7+m10≤6; when “m3” is 0, 1≤m4+m5+m8+m14≤4, and when “m3” is 1, 1≤m4+m5+m8+m14≤6; when “m11” is 0, 0≤m12+m13≤4, and when “m11” is 1, 0≤m12+m13≤6; also, 1≤m4+m12;
R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms; when “m5” is 2 or more, each R^{F1} may be the same as different from each other, when “m6” is 2 or more, each R^{F2} may be the same as or different from each other, and when “m7” is 2 or more, each R^{F3} may be the same as or different from each other;

R^{ct6} to R^{ct9} are a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “m8” is 2, two R^{ct6} may be the same as or different from each other, and two R^{ct6} may be bonded to each other to form a ring together with the carbon atoms to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two Roty may be bonded to each other to form a ring together with the carbon atoms to which they are bonded;
also, the aromatic rings directly bonded to S⁺ in the sulfonium cation may be bonded to each other to form a ring together with S⁺;
L^{D} and L^{E} are each independently a single bond, an ether bond, an ester bond, an amide bond, a sulfonate ester bond, a sulfonamide bond, a carbonate bond, or a carbamate bond; and
X¹² is a single bond, or a hydrocarbylene group having 1 to 40 carbon atoms and optionally containing a heteroatom.

In the general formula (Z-3), “m1” is 0 or 1. When “m1” is 0, the structure is a benzene ring, and when “m1” is 1, it is a naphthalene ring; however, from the viewpoint of solvent solubility, “m1” is preferably 0, giving a benzene ring. “m2” is 0 or 1. When “m2” is 0, the structure is a benzene ring, and when “m2” is 1, it is a naphthalene ring; however, from the viewpoint of solvent solubility, “m2” is preferably 0, giving a benzene ring. “m3” is 0 or 1. When “m3” is 0, the structure is a benzene ring, and when “m3” is 1, it is a naphthalene ring; however, from the viewpoint of solvent solubility, “m3” is preferably 0, giving a benzene ring.

In the general formula (Z-3), “m4” is an integer from 0 to 4. The larger the number of iodine atoms in the cationic structure, the higher the absorption, particularly for EUV, but the lower the solubility in solvents, and there is a concern that they may precipitate in the solvent. Therefore, “m4” is preferably an integer from 0 to 3, and more preferably 0, 1, or 2.

In the general formula (Z-3), “m5” is an integer from 0 to 4. From the viewpoint of raw material procurement, “m5” is preferably an integer from 0 to 3, and more preferably 0, 1, or 2. “m6” is an integer from 0 to 6. From the viewpoint of raw material procurement, “m6” is preferably an integer from 0 to 3, and more preferably 0, 1, or 2. “m7” is an integer from 0 to 6. From the viewpoint of raw material procurement, “m7” is preferably an integer from 0 to 3, and more preferably 0, 1, or 2.

In the general formula (Z-3), “m8” is 0, 1, or 2. From the viewpoint of raw material procurement, “m8” is preferably 0 or 1. “m9” is 0, 1, or 2. From the viewpoint of raw material procurement, “m9” is preferably 0 or 1. “m10” is 0, 1, or 2. From the viewpoint of raw material procurement, “m10” is preferably 0 or 1.

In the general formula (Z-3), “m11” is 0 or 1. When “m11” is 0, the structure is a benzene ring, and when “m11” is 1, it is a naphthalene ring; however, from the viewpoint of solvent solubility, “m11” is preferably 0, giving a benzene ring.

In the general formula (Z-3), “m12” is an integer from 0 to 4. The larger the number of iodine atoms in the cationic structure, the higher the absorption, particularly for EUV, but the lower the solubility in solvents, and there is a concern that they may precipitate in the solvent. Therefore, “m12” is preferably an integer from 0 to 3, and more preferably 0, 1, or 2.

In the general formula (Z-3), “m13” is 0, 1, or 2. From the viewpoint of raw material procurement, “m13” is preferably 0 or 1. “m14” is 0, 1, or 2. From the viewpoint of synthesis, “m14” is preferably 0 or 1. Provided that when “m1” is 0, 0≤m6+m9≤4, and when “m1” is 1, 0≤m6+m9≤6. When “m2” is 0, 0≤m7+m10≤4, and when “m2” is 1, 0≤m7+m10≤6. When “m3” is 0, 1≤m4+m5+m8+m14≤4, and when “m13” is 1, 1≤m4+m5+m8+m14≤6. When “m11” is 0, 0≤m12+m13≤4, and when “m11” is 1, 0≤m12+m13≤6. Also, 1≤m4+m12.

In the general formula (Z-3), R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Of these, a trifluoromethyl group, a trifluoromethoxy group, or a trifluorothiomethoxy group is preferable. When “m5” is 2 or more, each R^{F1} may be the same as or different from each other, when “m6” is 2 or more, each R^{F2} may be the same as or different from each other, and when “m7” is 2 or more, each R^{F3} may be the same as or different from each other.

In the general formula (Z-3), R^{ct6} to R^{ct9} are a halogen atom other than an iodine atom and fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl group and the hydrocarbyl moiety of the hydrocarbyloxy group and hydrocarbylthio group may be saturated or unsaturated, and may be linear, branched, or cyclic. Specific examples thereof include the same ones as those given as examples of the hydrocarbyl group represented by R¹ in the general formula (1). In addition, some or all of the hydrogen atoms of the hydrocarbyl group and the hydrocarbyl moiety of the hydrocarbyloxy group and the hydrocarbylthio group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In addition, when “m8” is 2, two R^{ct6} may be the same as or different from each other, and two R^{ct6} may be bonded to each other to form a ring together with the carbon atoms to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two R^{ct9} may be bonded to each other to form a ring together with the carbon atoms to which they are bonded. Specific examples of the ring formed in this case include a cyclopropane ring, a cyclobutane ring, a cyclopentane ring, a cyclohexane ring, a norbornane ring, and an adamantane ring. In addition, some or all of the hydrogen atoms in the ring may be substituted with groups containing heteroatoms such as an oxygen atom, a sulfur atom, a nitrogen atom, and a halogen atom, and some of the —CH₂— may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In addition, the aromatic rings directly bonded to S⁺ in the sulfonium cation represented by the general formula (Z-3) may bonded to each other to form a ring together with S⁺. In this case, specific examples of the ring structure include those represented by the following formulae.

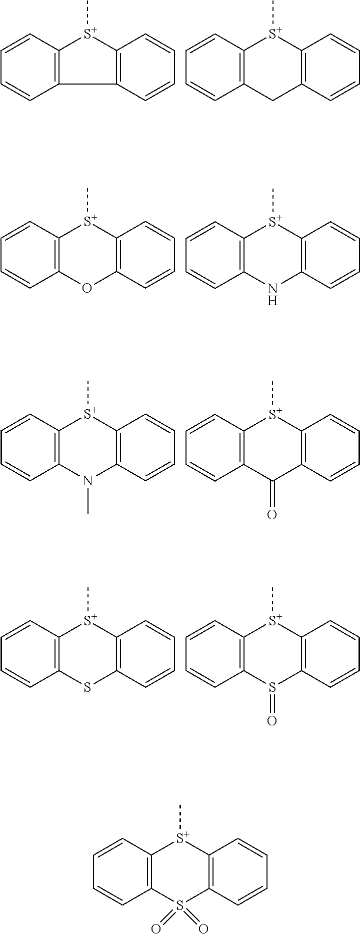

In the formulae, the dashed lines represent bonding point.

In the general formula (Z-3), L^{D} and L^{E} are each independently a single bond, an ether bond, an ester bond, an amide bond, a sulfonate ester bond, a sulfonate amide bond, a carbonate bond, or a carbamate bond. Of these, L′ is preferably a single bond, an ether bond, an ester bond, or a sulfonate ester bond, and more preferably an ester bond or a sulfonate ester bond. LE is preferably a single bond, an ether bond, or an ester bond, and more preferably a single bond.

In the general formula (Z-3), X¹² is a single bond, or a hydrocarbylene group having 1 to 40 carbon atoms and optionally containing a heteroatom. Examples of the hydrocarbylene group having 1 to 40 carbon atoms include the same ones as those given as specific examples of the hydrocalbylene group having 1 to 40 carbon atoms optionally containing a heteroatom represented by X^{L}, but are not limited thereto.

The sulfonium cation represented by the general formula (Z-3) is preferably one represented by the following general formula (Z-3-1).

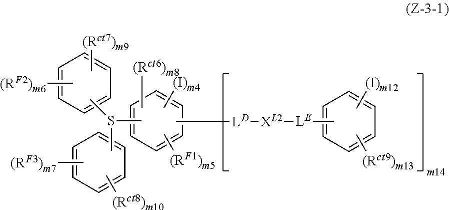

In the formula, “m4” to “m10”, “m12” to “m14”, R^{F1} to R^{F3}, R^{ct6} to R^{ct9}, LD, LE, and X¹² are as defined above.

The sulfonium cation represented by the general formula (Z-3-1) is preferably one represented by the following general formula (Z-3-2).

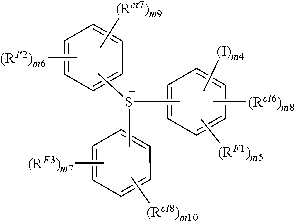

In the formula, “m4” to “m10”, R^{F1} to R^{F3}, and R^{ct6} to R^{ct8} are as defined above.

Specific examples of the sulfonium cation represented by the general formula (Z-3) include, but are not limited to, those shown below. In addition, in the following formulae, Me is a methyl group.

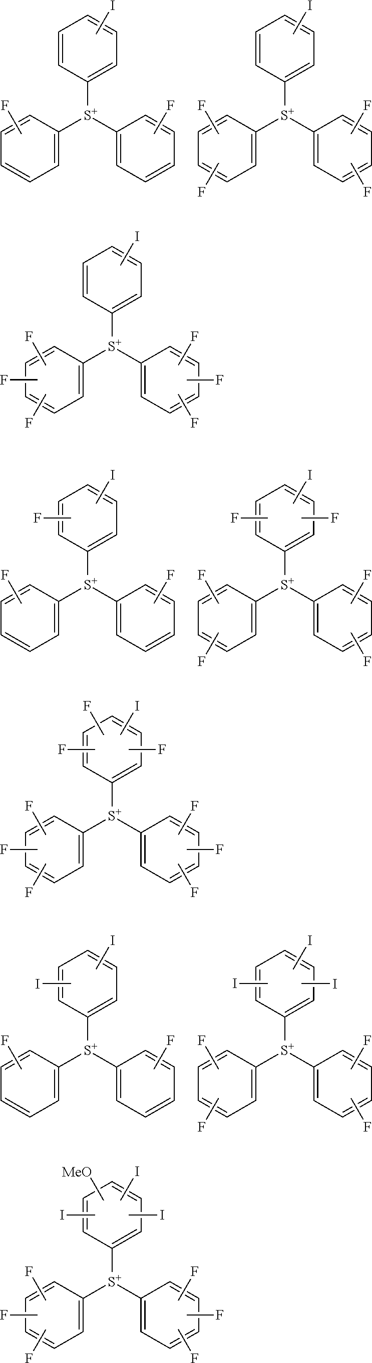

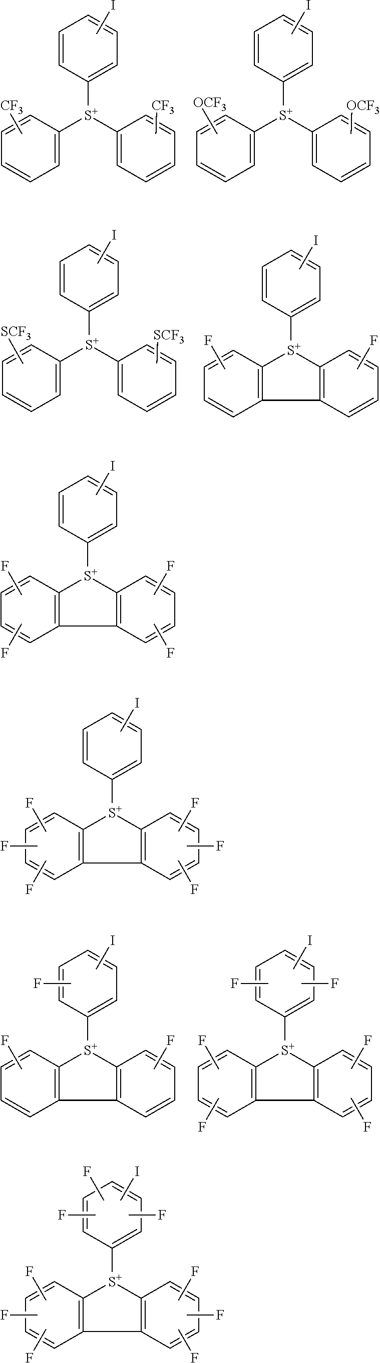

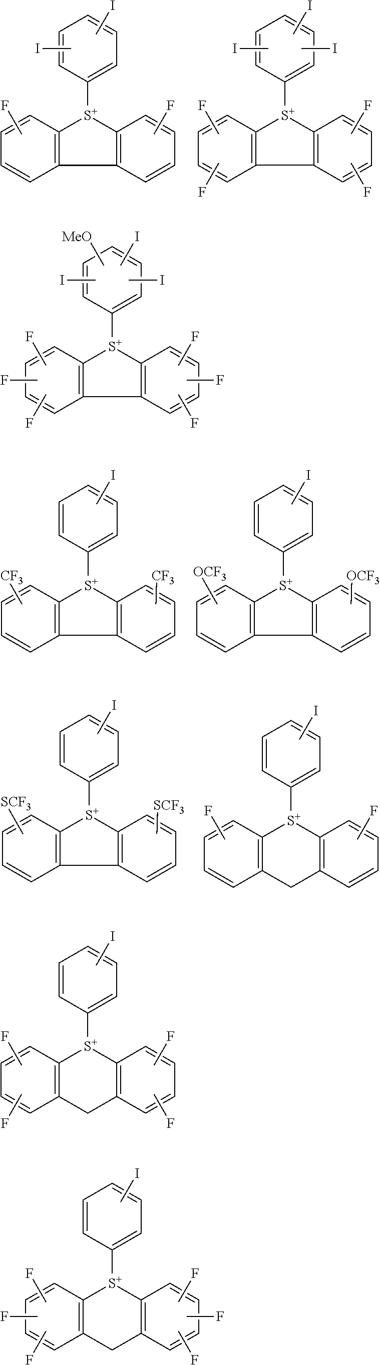

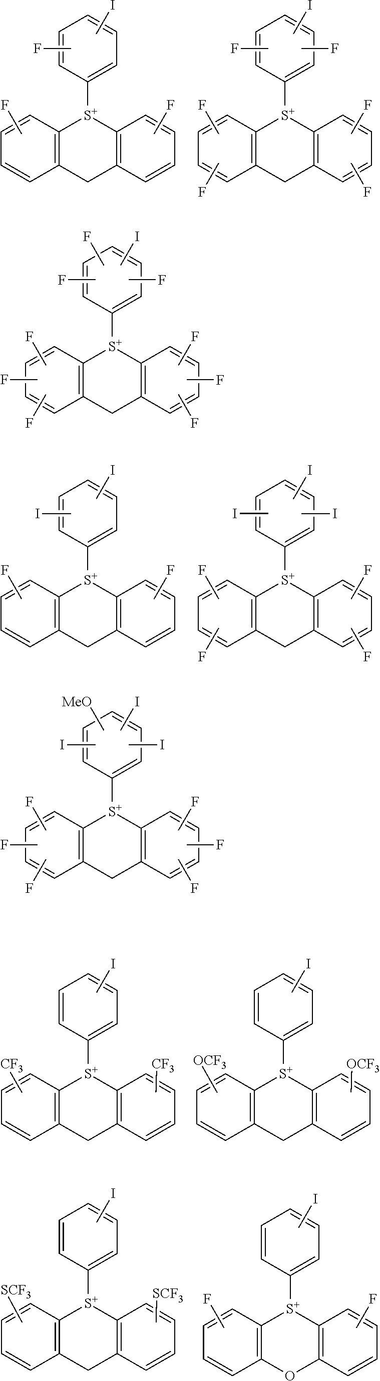

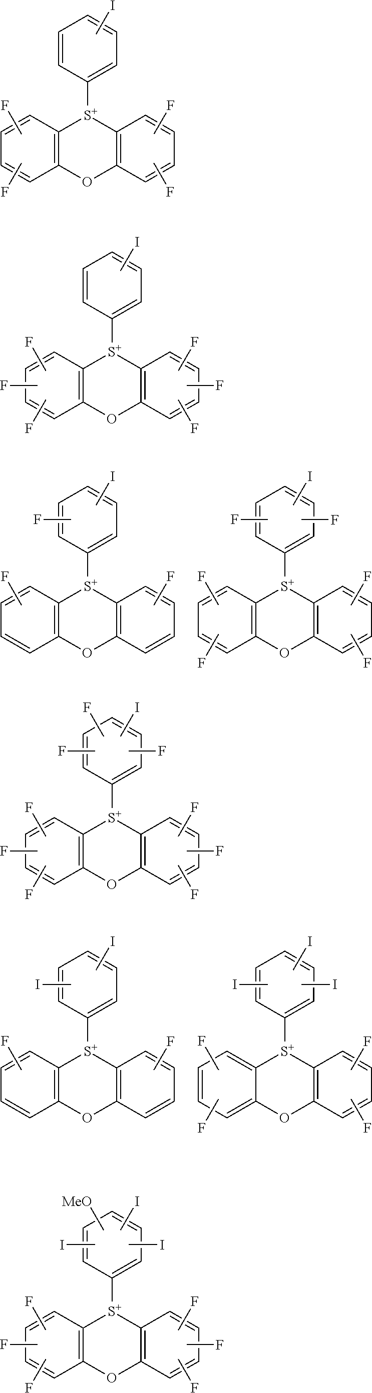

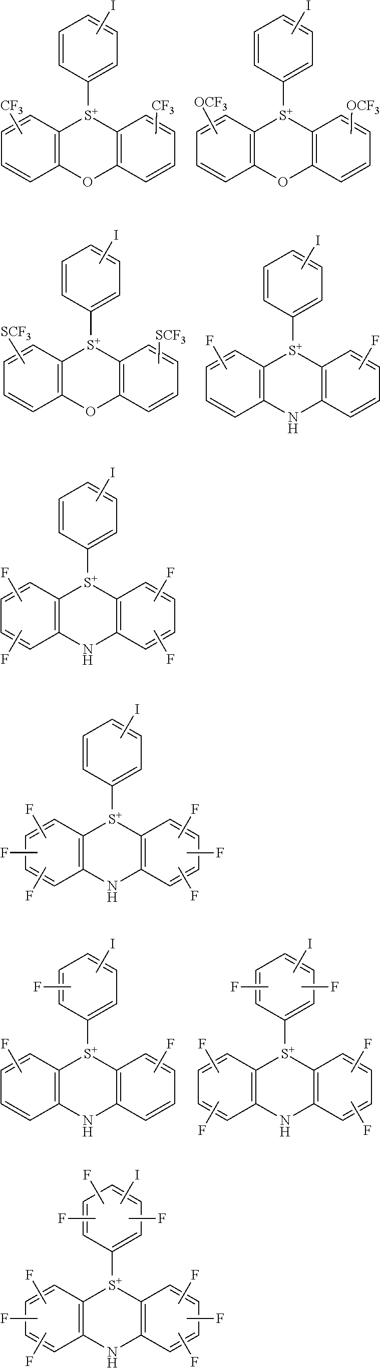

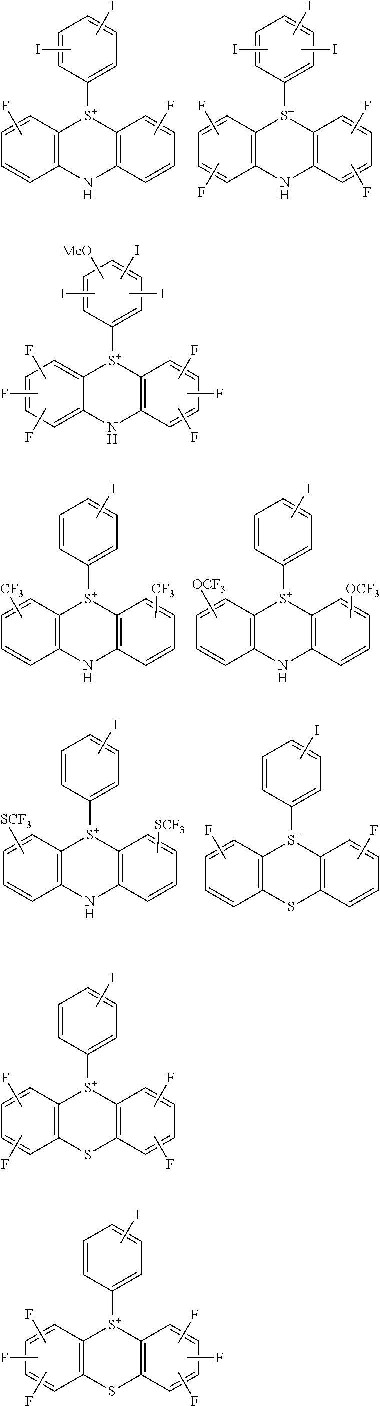

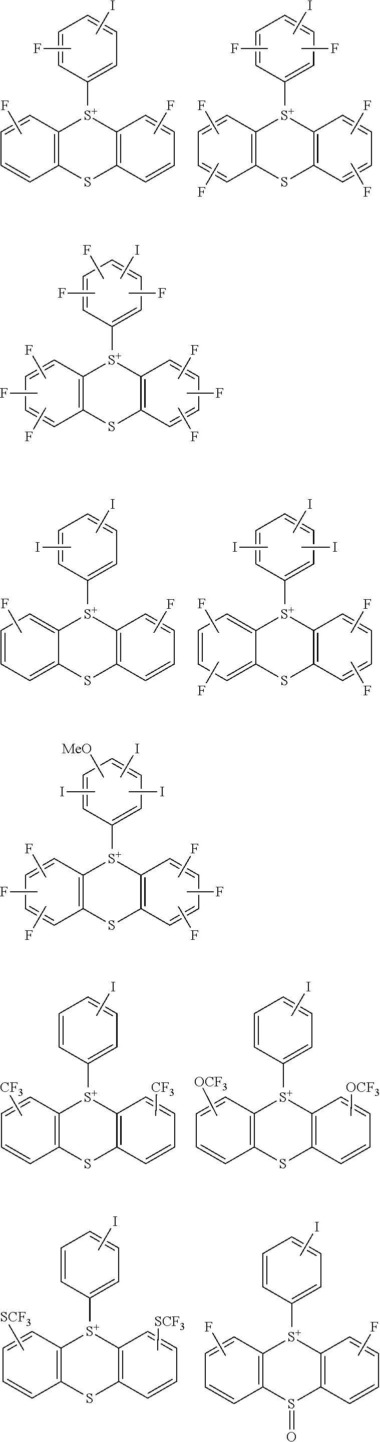

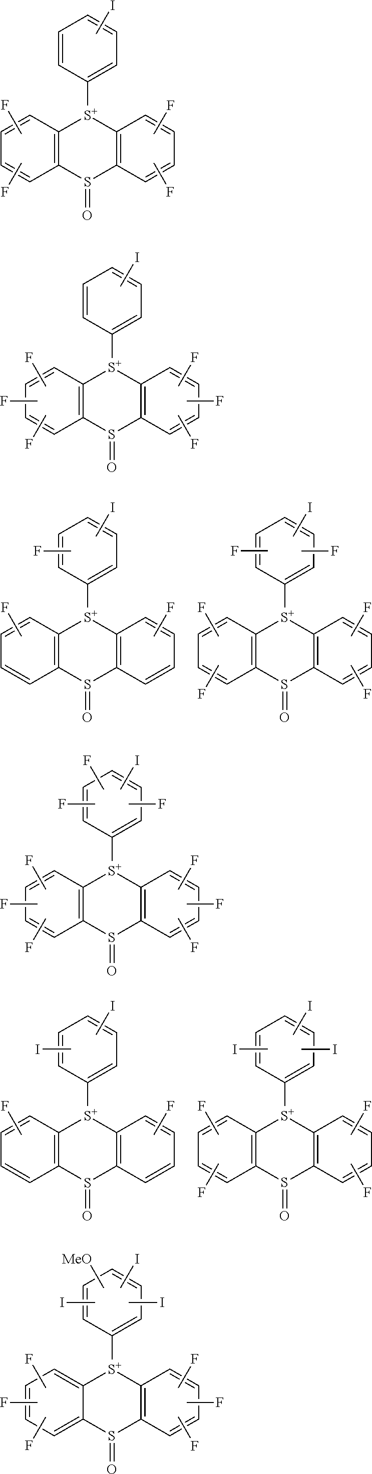

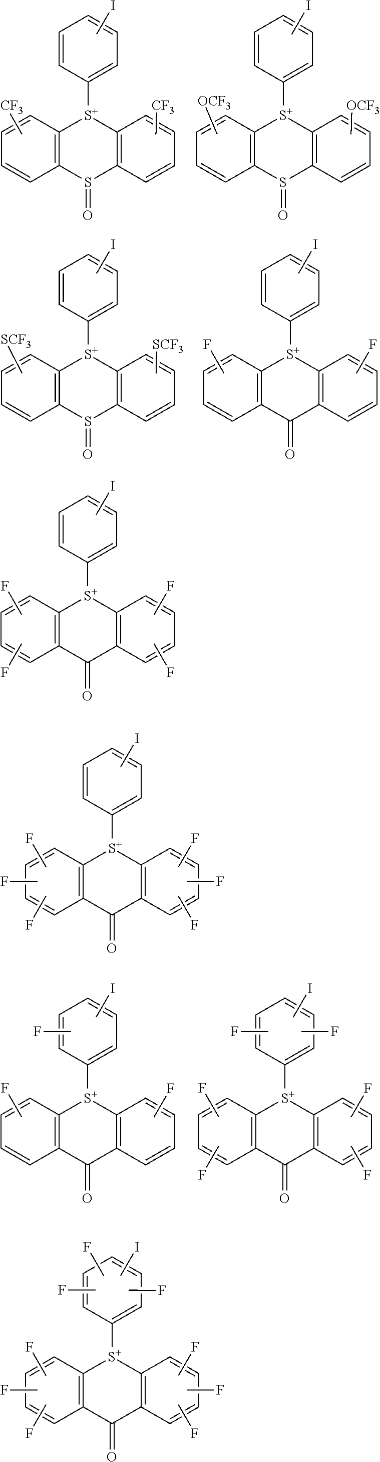

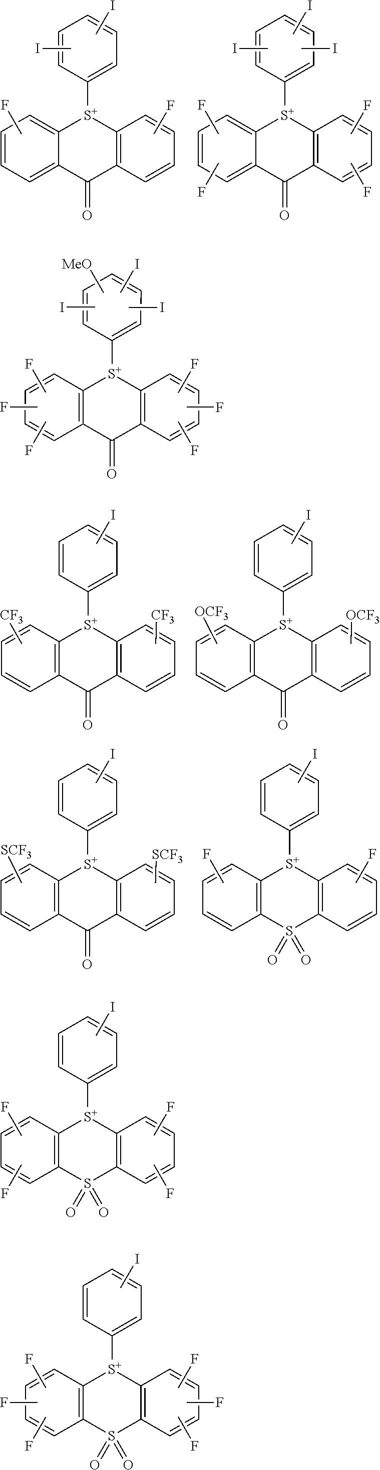

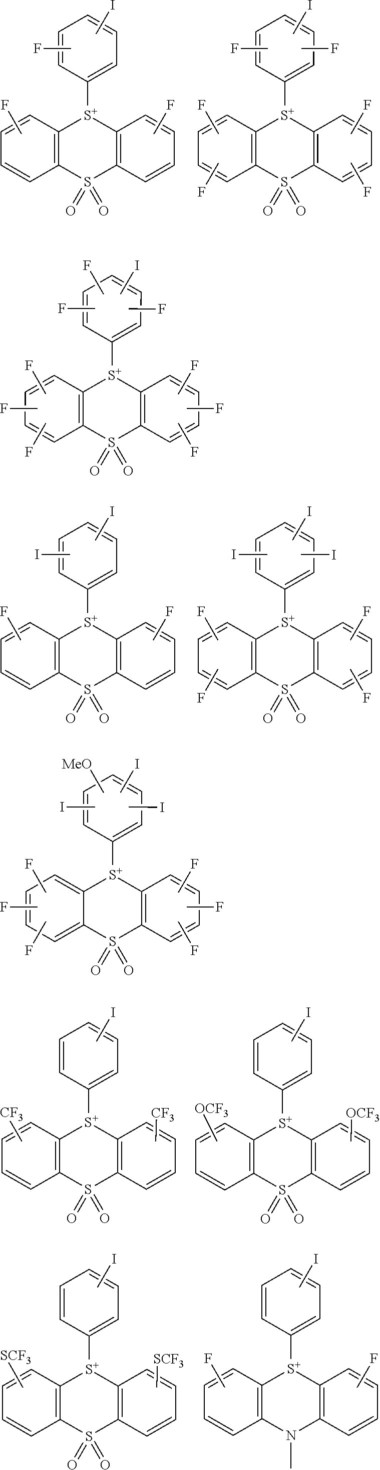

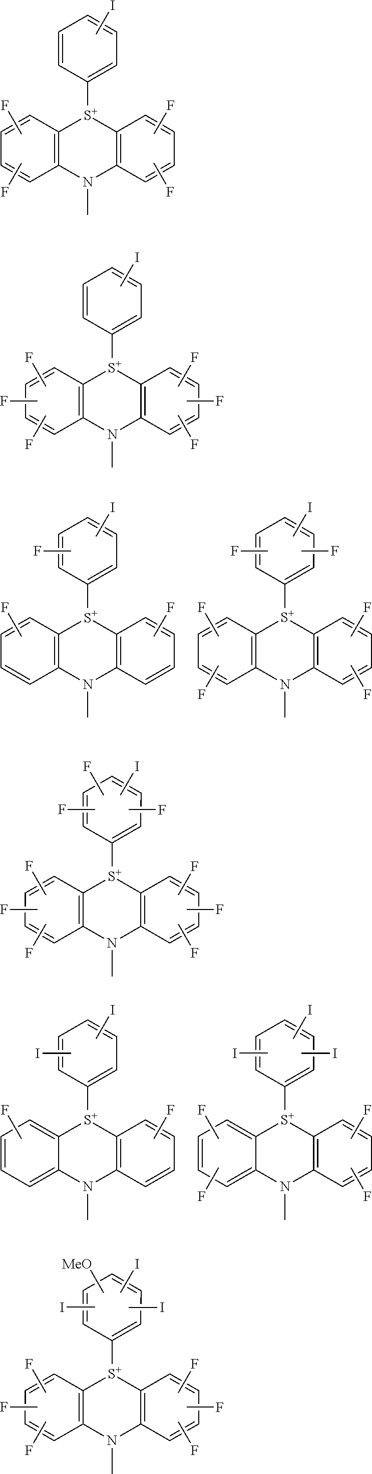

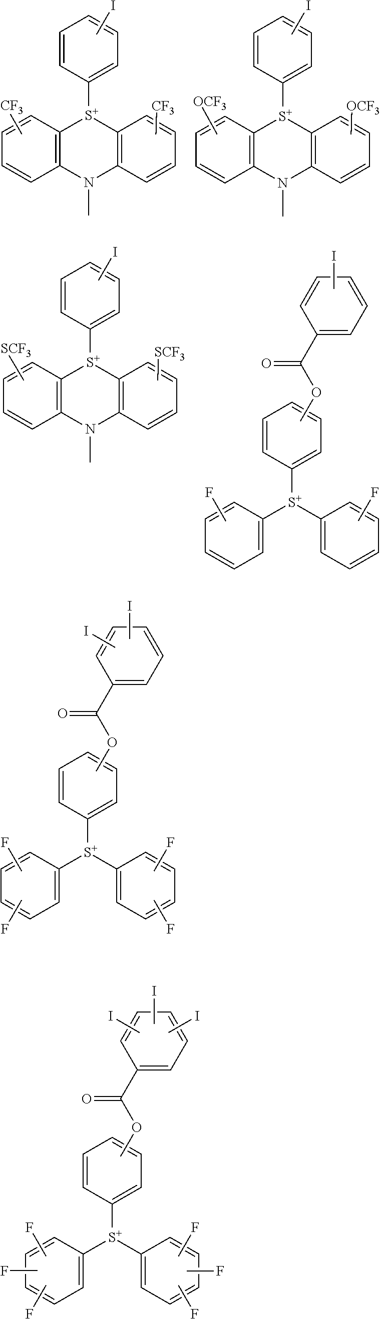

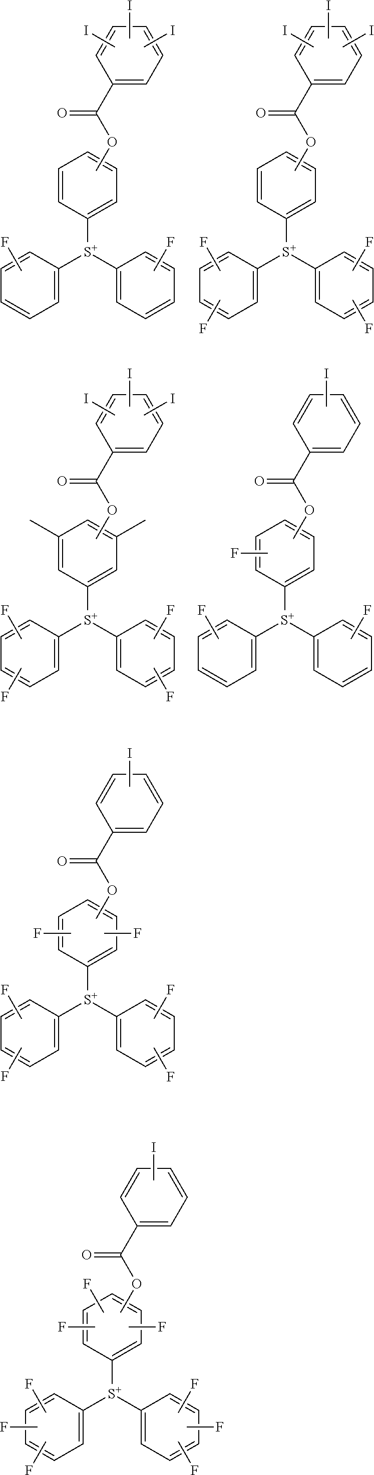

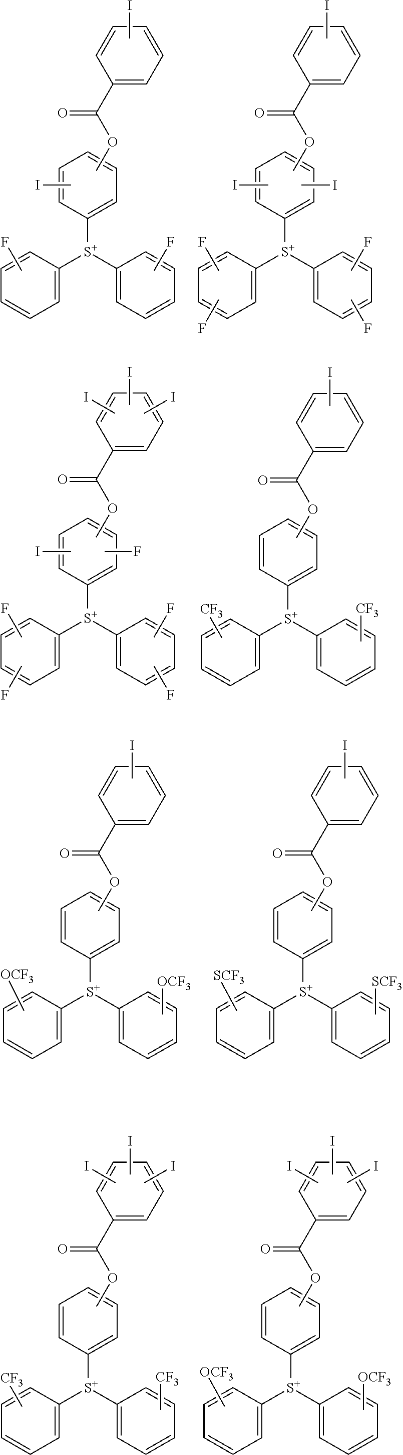

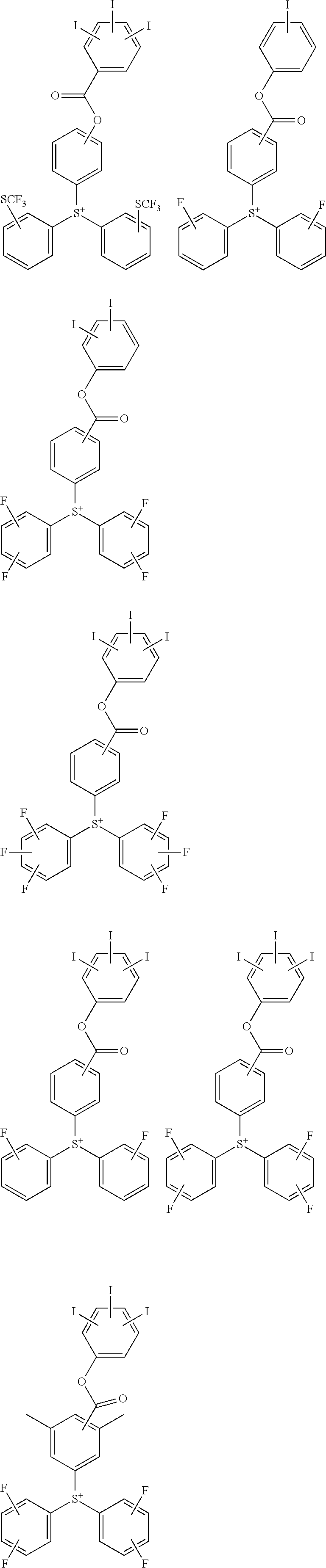

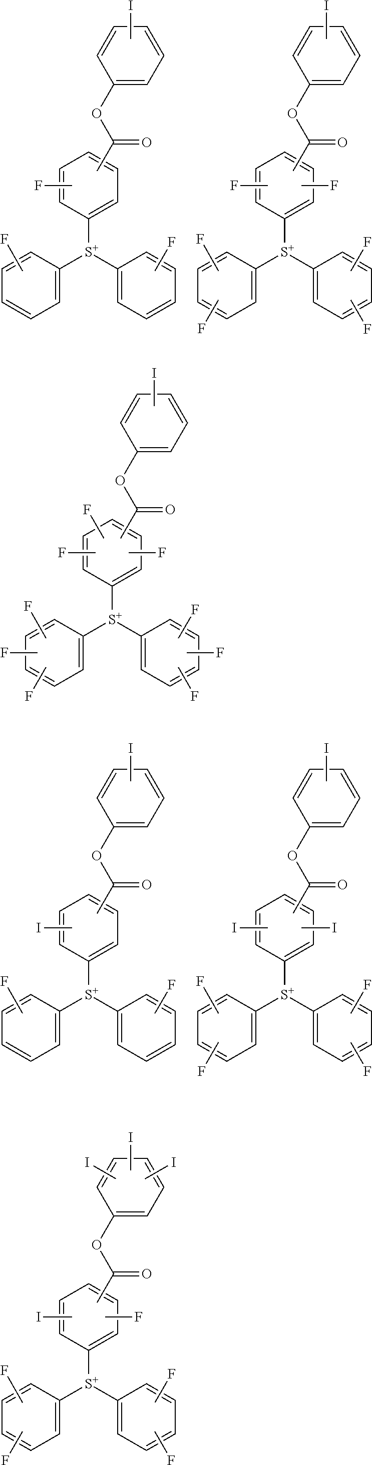

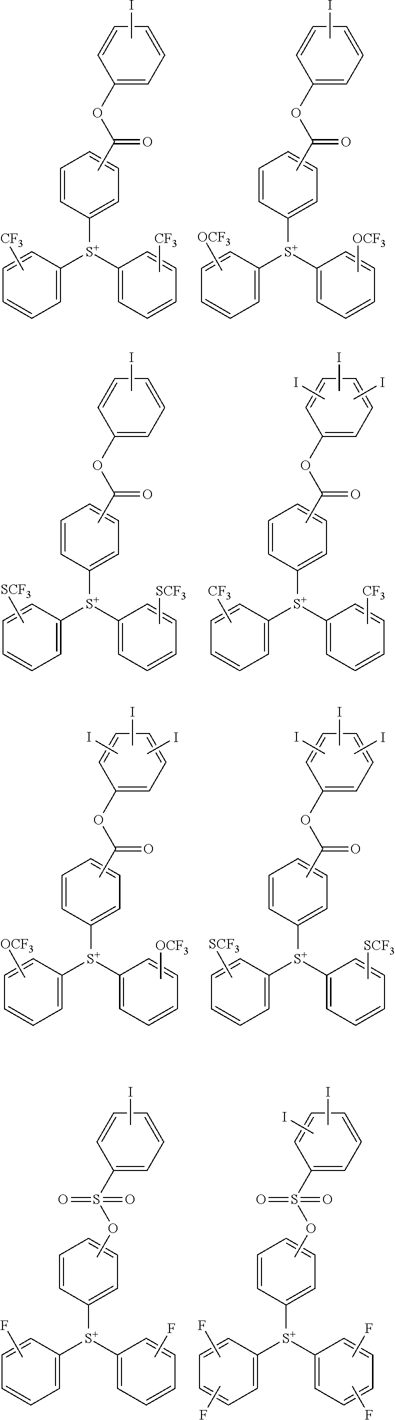

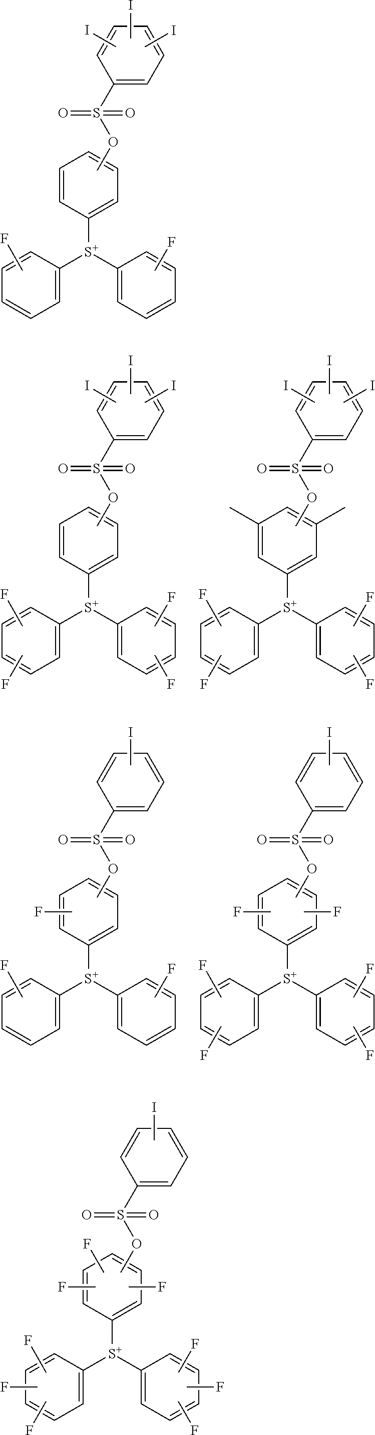

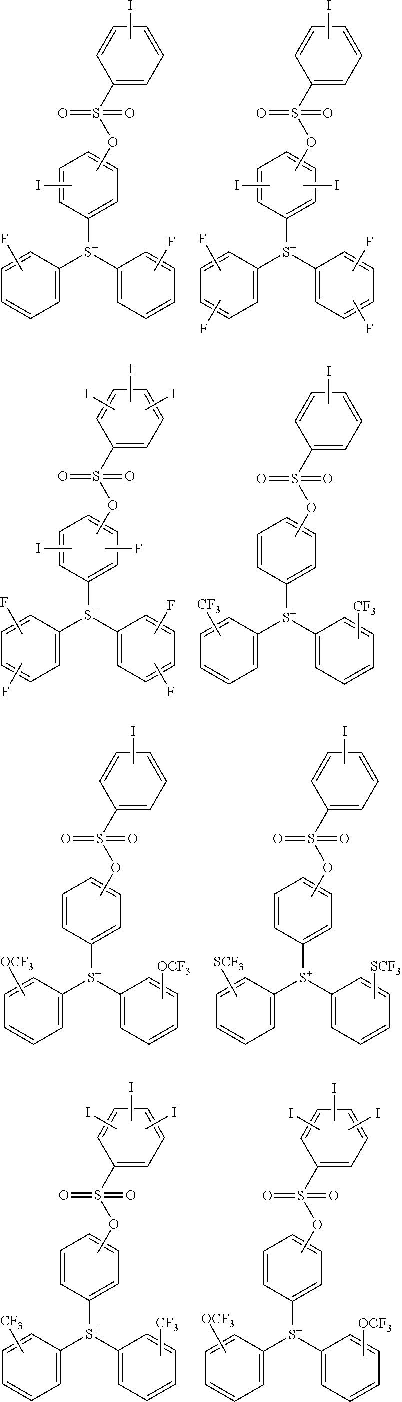

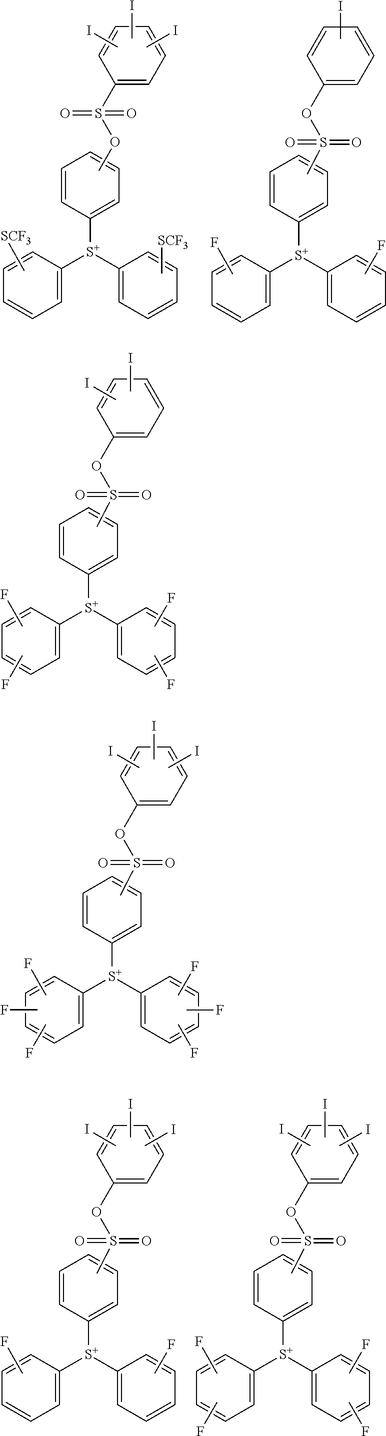

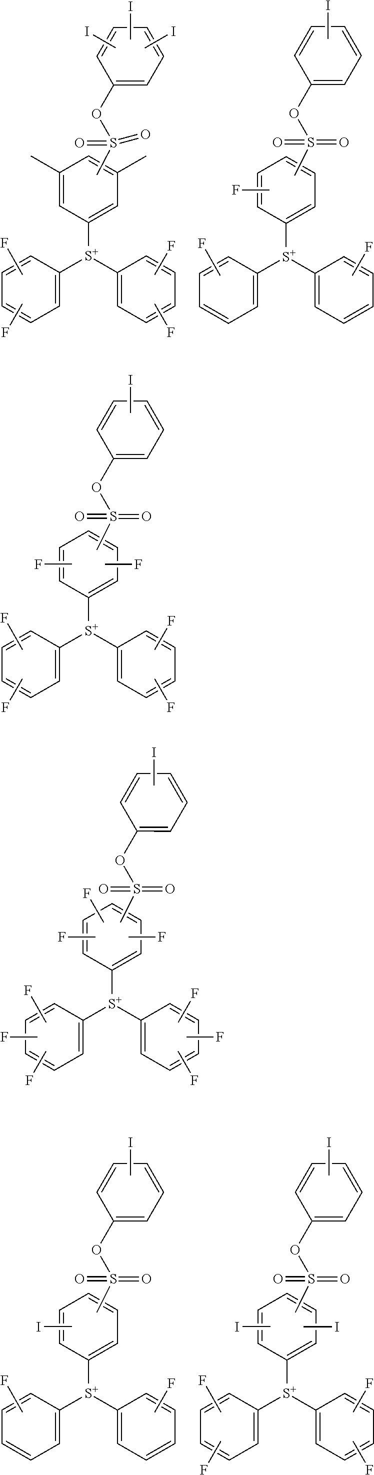

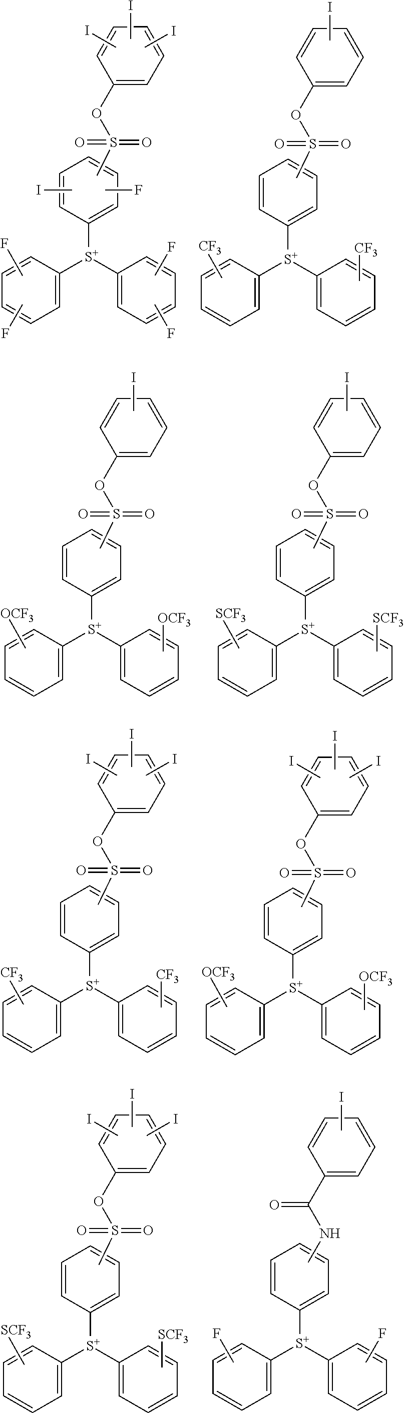

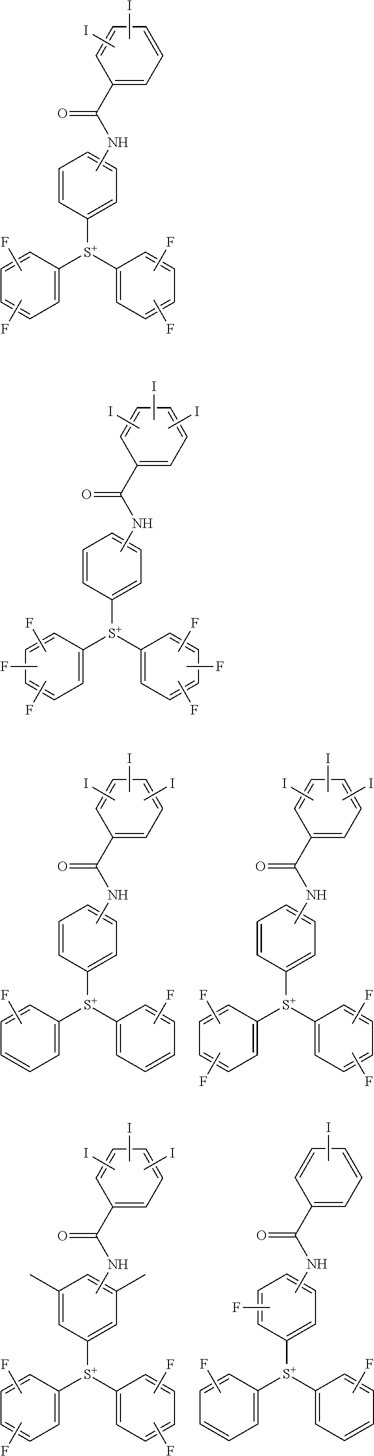

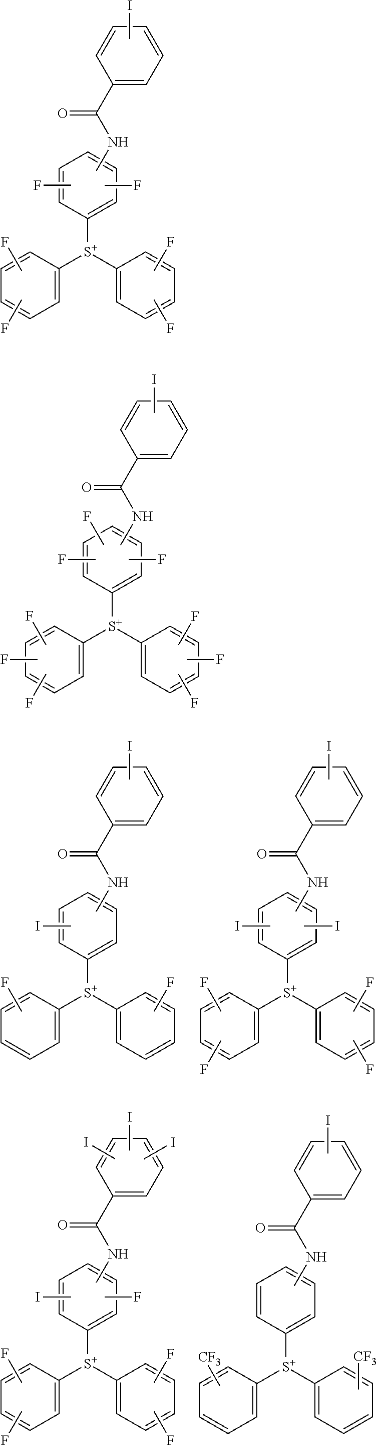

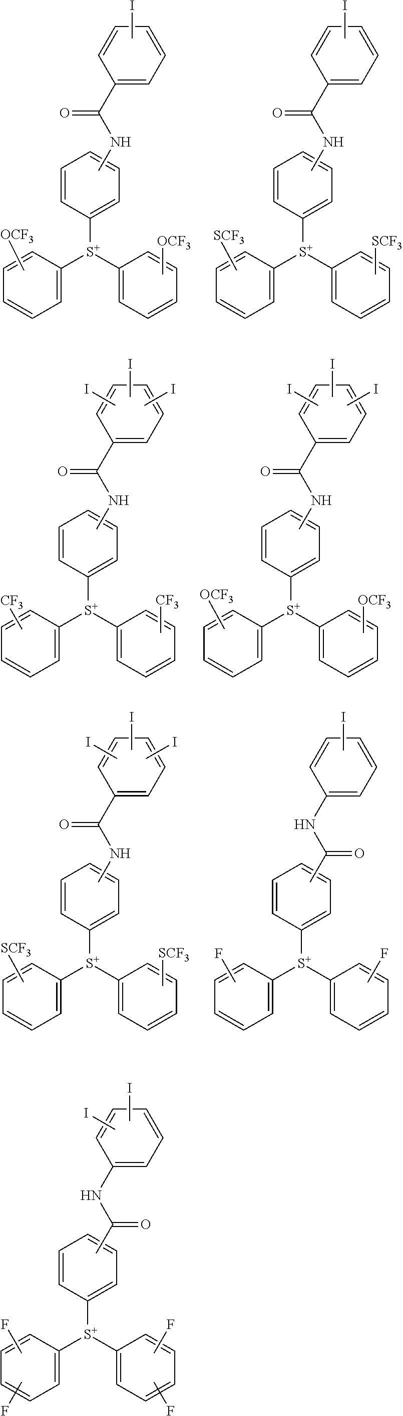

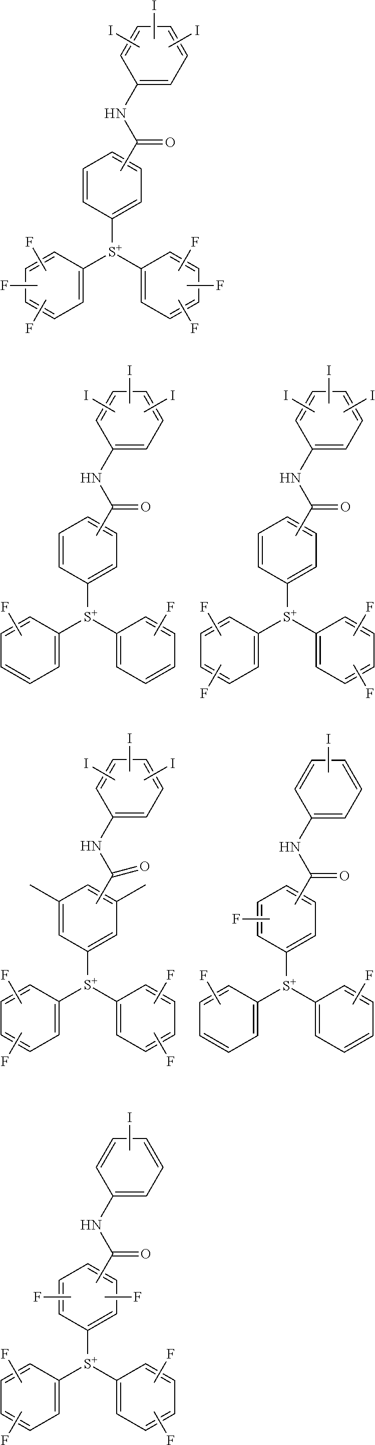

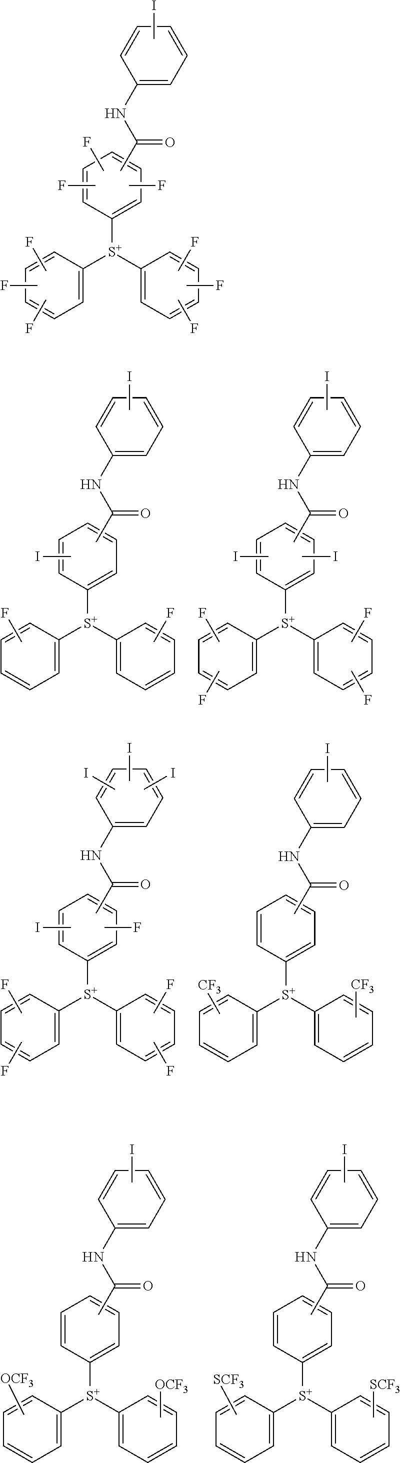

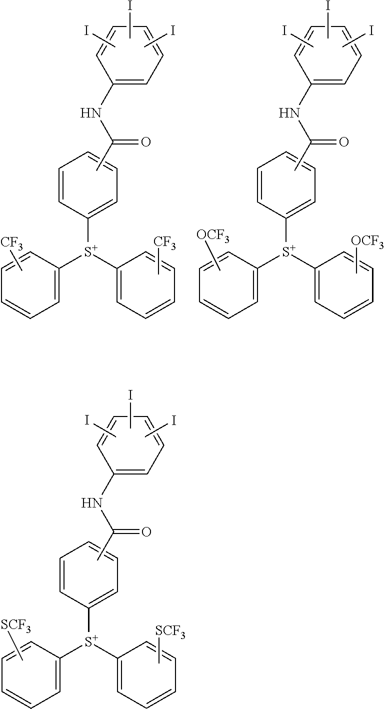

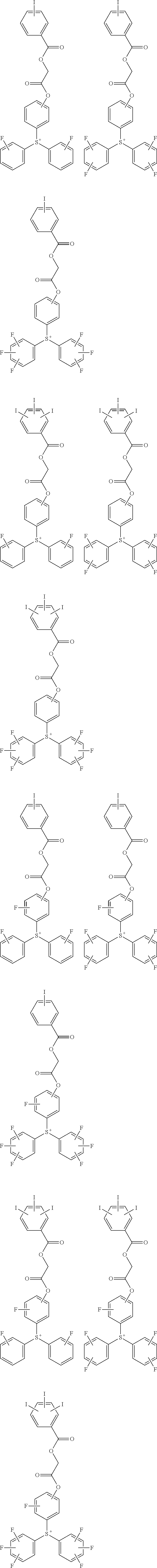

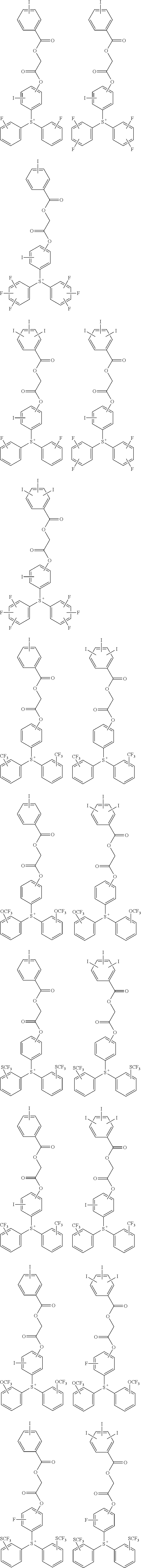

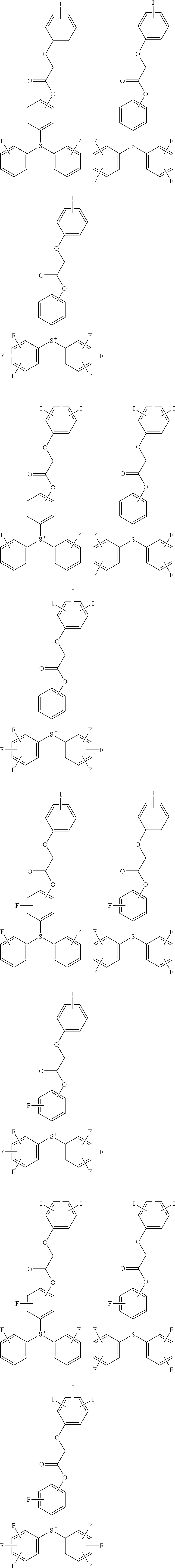

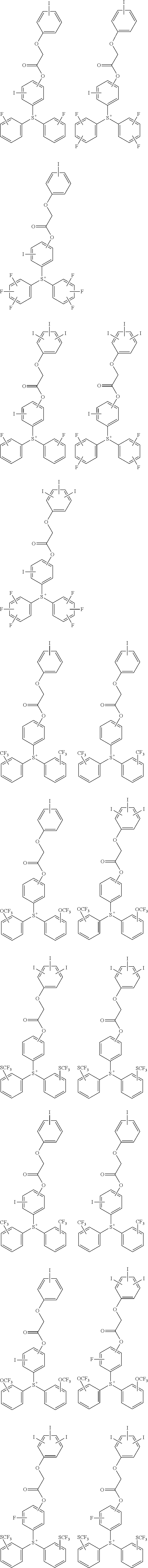

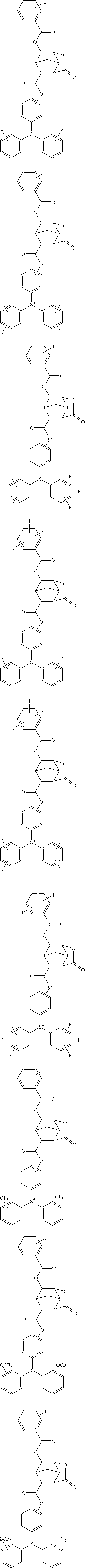

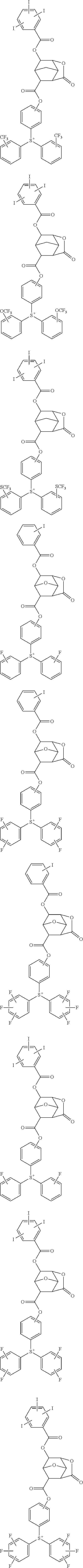

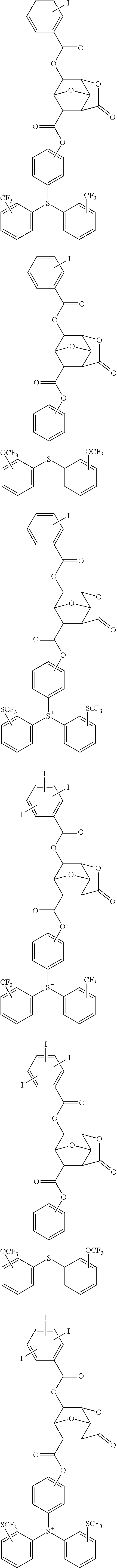

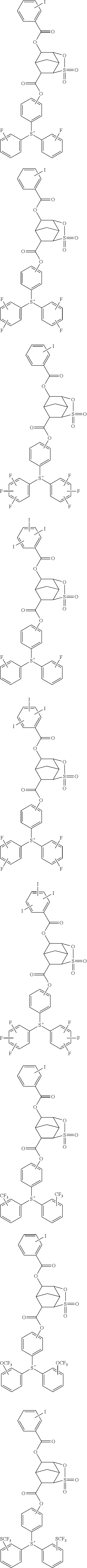

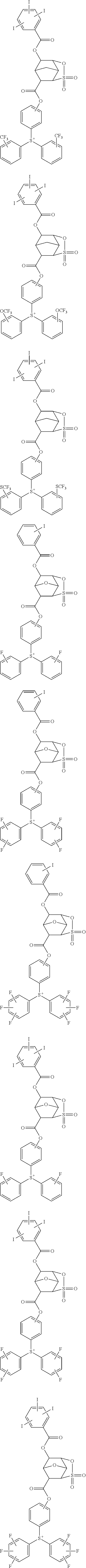

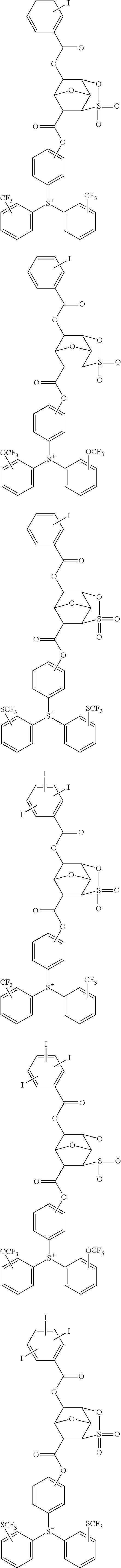

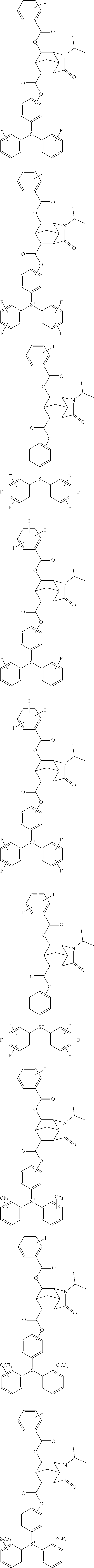

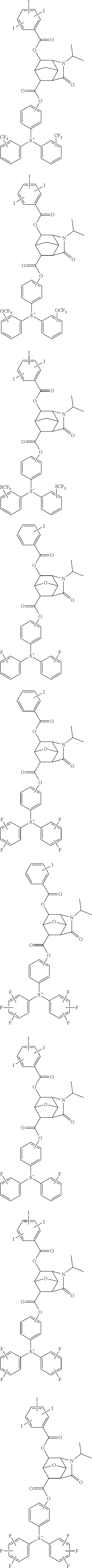

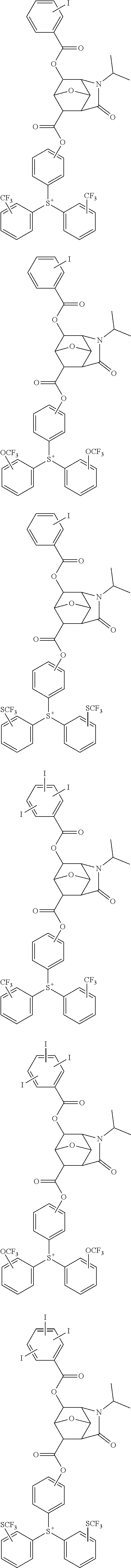

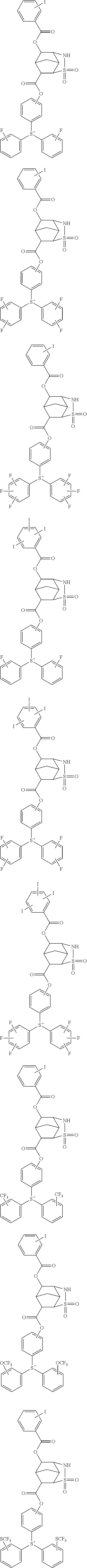

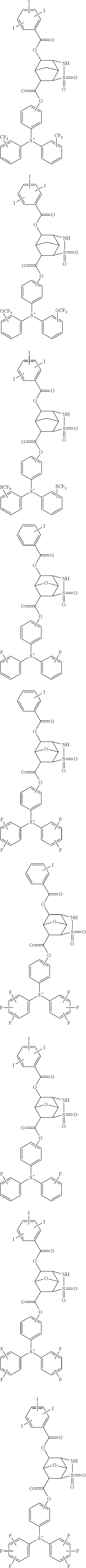

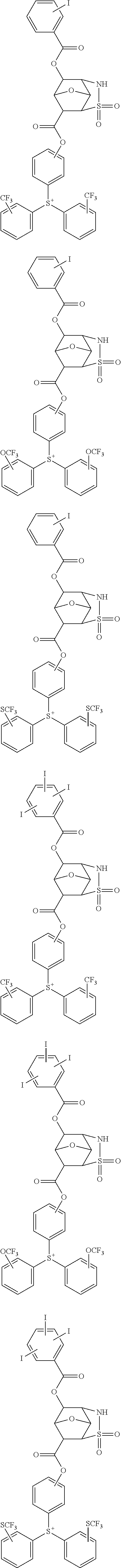

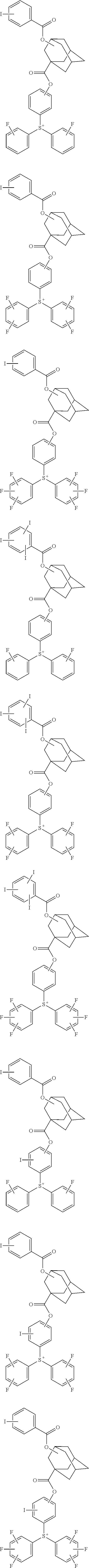

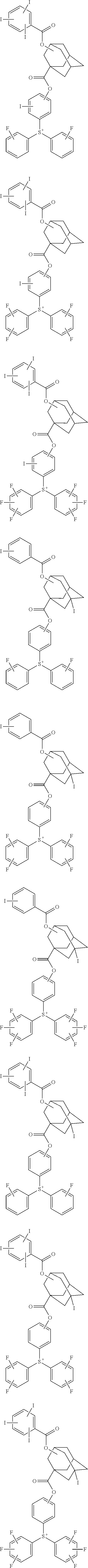

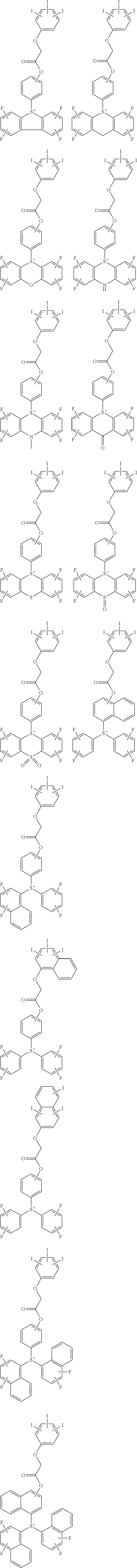

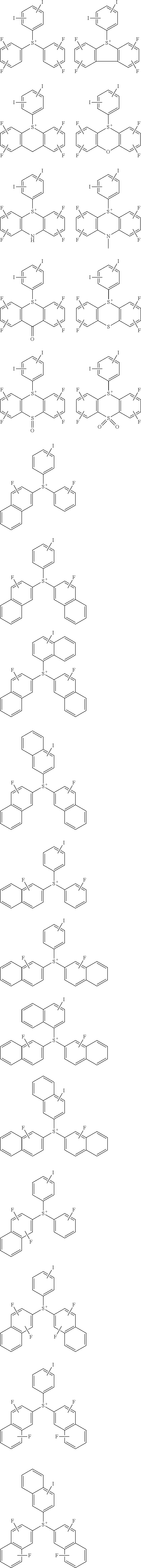

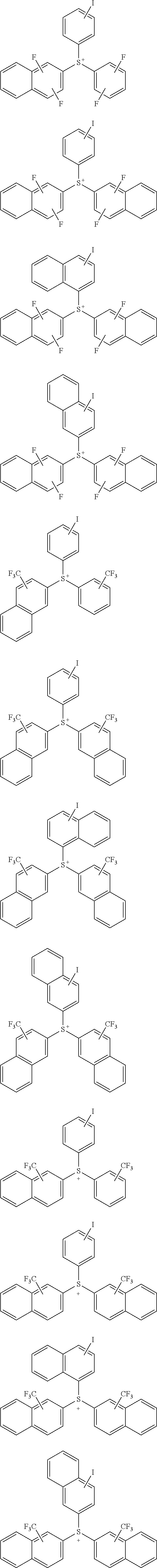

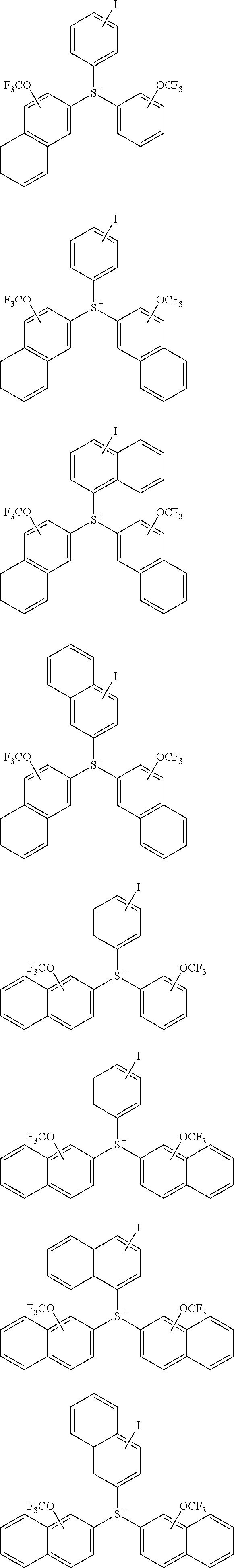

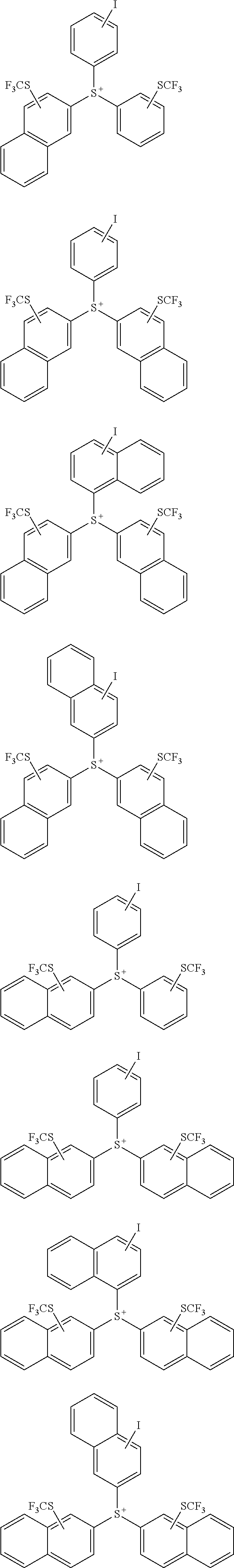

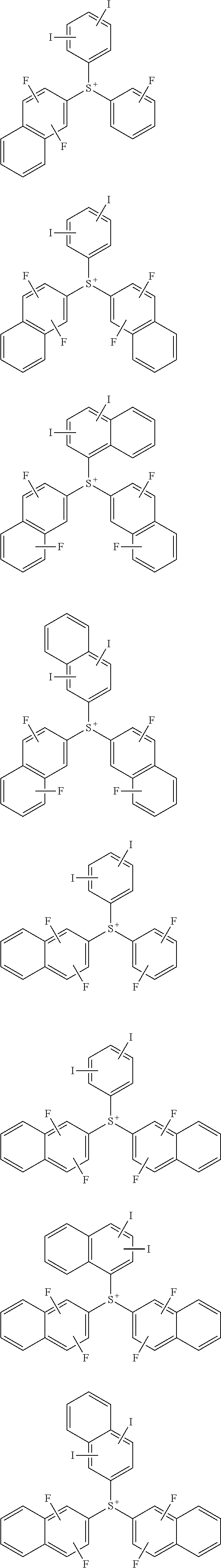

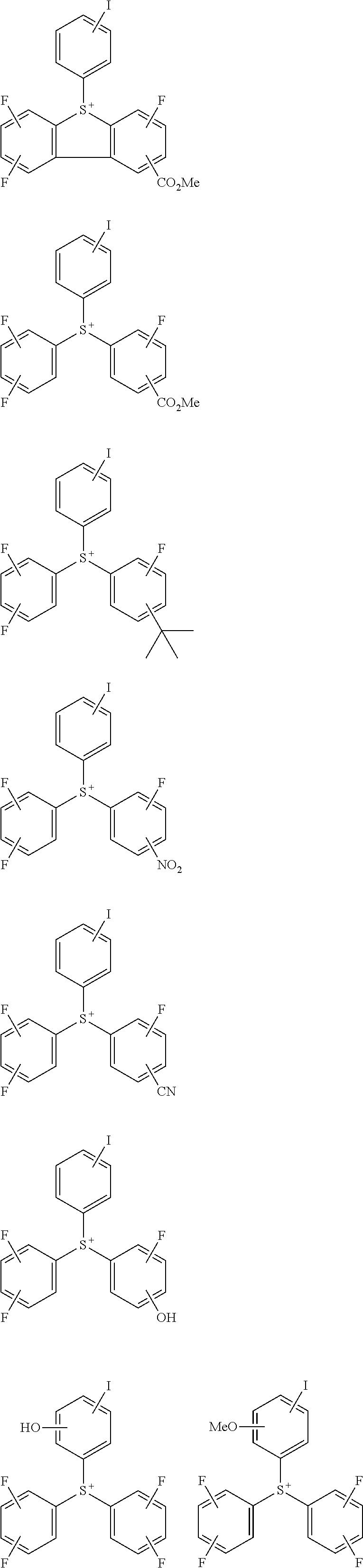

Specific examples of the onium salt of the present invention include any combination of the above-mentioned anions and cations.

The onium salt of the present invention can be synthesized by a known method. As an example, a method for manufacturing the onium salt represented by the following general formula (PAG-A-ex) will be described, but the synthesis method is not limited thereto.

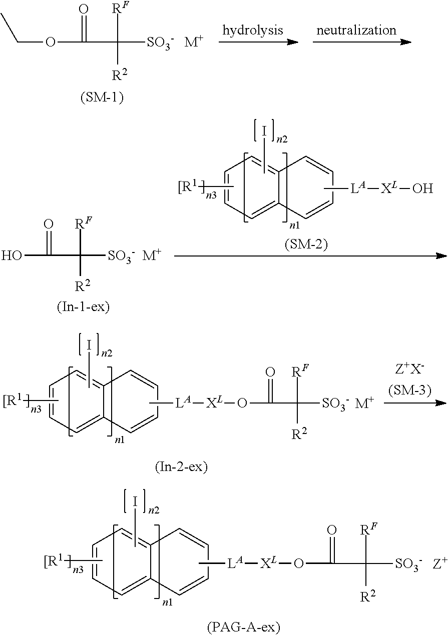

In the formulae, R¹, R², R^{F}, L^{A}, X^{L}, “n1” to “n3”, and Z⁺ are as defined above. M⁺ is a counter cation. X⁻ is a counter anion.

The first step is a step of obtaining the intermediate In-1-ex by a hydrolysis reaction and then a neutralization reaction of the ester moiety of the raw material SM-1, which is commercially available or obtained by a known synthesis method. The hydrolysis of the ester of the raw material SM-1 is preferably carried out under alkaline conditions, and the base used is preferably an alkali metal hydroxide such as sodium hydroxide or potassium hydroxide. The raw material SM-1 is dissolved in a solvent such as tetrahydrofuran or 1,4-dioxane, and a base is added to carry out the reaction. To ensure the reaction proceeds smoothly, the reaction can be heated as necessary. From the viewpoint of yield, it is desirable to follow the reaction by silica gel thin layer chromatography (TLC) to complete the reaction, but the reaction time is usually about 4 to 12 hours. After checking the progress of the reaction, a small excess of acid over the base is added to carry out a neutralization reaction. The acid used is preferably a strong acid, with specific examples including hydrochloric acid, sulfuric acid, and nitric acid. After the neutralization reaction, the solvent is concentrated to obtain the intermediate In-1-ex and, if necessary, the obtained intermediate In-1-ex may be purified by conventional methods such as chromatography and recrystallization.

The second step is a step of obtaining the intermediate In-2-ex by the reaction of the intermediate In-1-ex and the raw material SM-2. When directly forming an ester bond from the carboxy group of the intermediate In-1-ex and the hydroxy group of the raw material SM-2, various condensation agents can be used. Condensation agents that can be used include N,N′-dicyclohexylcarbodiimide, N,N′-diisopropylcarbodiimide, 1-[3-(dimethylamino) propyl]-3-ethylcarbodiimide, 1-ethyl-3-(3-dimethylaminopropyl) carbodiimide hydrochloride, etc., but from the viewpoint of ease of removal of the urea compound generated as a by-product after the reaction, it is preferable to use 1-ethyl-3-(3-dimethylaminopropyl) carbodiimide hydrochloride. The reaction is carried out by dissolving the intermediate In-1-ex and the raw material SM-2 in a halogen-based solvent such as methylene chloride, and adding a condensation agent. The reaction rate can be improved by adding 4-dimethylaminopyridine as a catalyst. From the viewpoint of yield, it is desirable to follow the reaction by silica gel thin layer chromatography (TLC) to complete the reaction, but the reaction time is usually about 12 to 24 hours. After the reaction is stopped, the by-product urea compound is removed by filtration or washing with water if necessary, the reaction solution is then be subjected to normal aqueous work-up, and then the intermediate In-2-ex can be obtained. If necessary, the obtained intermediate In-2-ex can be purified by conventional methods such as chromatography and recrystallization.

The third step is a step of salt-exchanging the obtained intermediate In-2-ex with an onium salt (raw material SM-3) represented by Z⁺X⁻ to obtain an onium salt (PAG-A-ex). Note that, as X⁻, a chloride ion, a bromide ion, an iodide ion, or a methyl sulfate anion is preferable, because the exchange reaction easily proceeds quantitatively. From the viewpoint of yield, it is desirable to monitor the reaction by TLC to complete the reaction, but the reaction time is usually about 4 to 12 hours. The onium salt type monomer (PAG-A-ex) can be obtained from the reaction mixture by normal aqueous work-up. If necessary, it can be purified by a normal method such as chromatography or recrystallization.

In the above scheme, the ion exchange in the third step can be easily carried out by a known method; for example, see JP2007-145797A.

Note that, the above manufacturing method is merely one example, and the manufacturing method of the onium salt of the present invention is not limited to this.

The structural features of the onium salt of the present invention include that it has an aromatic ring containing an iodine atom and an alkane sulfonate anion having only one fluorine atom or one fluorine-containing alkyl group at the α-position from the sulfo group. In addition, it preferably further has a carbonyl group at the β-position from the sulfo group. Iodine atoms have extremely high absorption of EUV light with a wavelength of 13.5 nm, so secondary electrons are generated during exposure, and the energy of the secondary electrons is transferred to the acid generator, promoting its decomposition, and thereby resulting in high sensitivity. In addition, since iodine atoms are an element with a large molecular weight, substituting them into an anion increases its molecular weight, thereby reducing acid diffusion. In addition, the carbonyl group at the β-position, the fluorine atom at the α-position, or the fluorine-containing alkyl group at the α-position of the sulfo group of the generated acid can moderately increase the acidity of the generated acid due to their respective electron-withdrawing effects. Although the acidity is lower than that of α,α-difluorosulfonic acid, it is still sufficient to deprotect the acid-labile groups of the base polymer. Therefore, it is preferable to combine the onium salt of the present invention with a base polymer containing an acid-labile unit whose activation energy in the deprotection reaction is not so high. Specifically, it is preferable to select a deprotection reaction with a relatively low activation energy and is driven by the generation of an allyl cation or benzyl cation. When using an acid-labile group that generates a simple tertiary carbocation, the group is preferably a tertiary ester or tertiary ether having a cyclic structure. Due to the above synergistic effects, when the onium salt of the present invention is used, excessive acid diffusion is suppressed while maintaining high sensitivity, so it is possible to form patterns that have high dissolution contrast, have excellent line pattern LWR and hole pattern CDU, and are resistant to pattern collapse, making it particularly suitable as a positive resist composition.

### [Photo-Acid Generator]

In addition, the present invention provides a photo-acid generator, wherein the photo-acid generator is composed of the onium salt described above. That is, the onium salt can be suitably used as a photo-acid generator.

### [Chemically Amplified Resist Composition]

The chemically amplified resist composition of the present invention contains a photo-acid generator (A) containing the photo-acid generator consisting of an onium salt represented by general formula (1). That is, the present invention provides a chemically amplified resist composition, wherein the chemically amplified resist composition contains the above-described photo-acid generator.

In the chemically amplified resist composition of the present invention, the content of component (A), namely, the photo-acid generator consisting of an onium salt represented by general formula (1), is preferably 0.1 to 40 parts by mass, and more preferably 0.5 to 30 parts by mass, per 80 parts by mass of the base polymer described below. When the content of component (A) is within the above range, the sensitivity and resolution are good, and there is no risk of problems with foreign matter occurring after development of the resist film or during stripping, so it is preferable. The photo-acid generator (A) may be used alone or in a combination of two or more types.

### [Base Polymer (B)]

The chemically amplified resist composition of the present invention may contain a base polymer as component (B). The base polymer (B) contains a repeating unit represented by the following general formula (a1)(hereinafter also referred to as repeating unit-a1) and/or a repeating unit represented by the following general formula (a2)(hereinafter also referred to as repeating unit-a2).

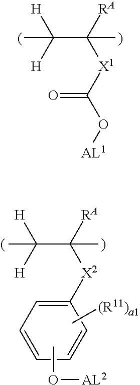

In the formulae, “a1” is an integer from 0 to 4;
R^{A} is each independently a hydrogen atom, a fluorine atom, a methyl group, or a trifluoromethyl group;
X¹ is a single bond, a phenylene group, a naphthylene group, *—C(═O)—OX¹¹—, or *—C(═O)—NH—X¹¹, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ is a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² is a single bond, *—C(═O)—O—, or *—C(═O)—NH—;
“*” represents a bond to a main chain carbon atom;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2, 3, or 4, each R¹¹ may be the same as or different from each other; and
A^{L1} and A^{L2} are each independently an acid-labile group.

In the general formulae (a1) and (a2), R^{A} is each independently a hydrogen atom, a fluorine atom, a methyl group, or a trifluoromethyl group.

In the general formula (a1), X¹ is a single bond, a phenylene group, a naphthylene group, *—C(═O)—O—X¹¹—, or *—C(═O)—NH—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom. X¹¹ is a saturated hydrocarbylene group having 1 to 10 carbon atoms, a phenylene group, or a naphthylene group, and the saturated hydrocarbylene group may contain a hydroxy group, an ether bond, an ester bond, or a lactone ring. “*” represents a bond to a main chain carbon atom.

In the general formula (a2), X² is a single bond, *—C(═O)—O—, or *—C(═O)—NH—. “*” represents a bond to a main chain carbon atom. “a1” is an integer from 0 to 4, preferably 0 or 1. R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. When “a1” is 2, 3, or 4, each R¹¹ may be the same as or different from each other.

In the general formulae (a1) and (a2), A^{L1} and A^{L2} are each independently an acid-labile group. Specific examples of the acid-labile group include those described in JP2013-080033A and JP2013-083821A.

Typical examples of the acid-labile group include those represented by the following general formulae (AL-3) to (AL-5).

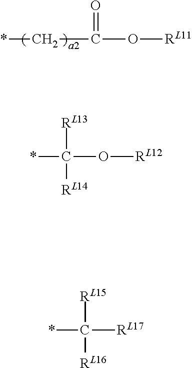

In the formulae, the dashed lines represent bonding point.

In the general formulae (AL-3) and (AL-4), R^{L11} and R^{L12} are each independently a hydrocarbyl group having 1 to 40 carbon atoms and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms.

In the general formula (AL-3), “a2” is an integer from 0 to 10, and preferably an integer from 1 to 5.

In the general formula (AL-4), R^{L13} and R^{L14} are each independently a hydrogen atom or a hydrocarbyl group having 1 to 20 carbon atoms and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms. In addition, any two of the R^{L12}, R^{L13}, and R^{L14} may be bonded to each other to form a ring having 3 to 20 carbon atoms together with the carbon atom or the carbon atom and oxygen atom to which they are bonded. The ring is preferably a ring having 4 to 16 carbon atoms, and particularly preferably an alicyclic ring.

In the general formula (AL-5), R^{L15}, R^{L16}, and R^{L17} are each independently a hydrocarbyl group having 1 to 20 carbon atoms and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms. In addition, any two of R^{L15}, R^{L16}, and R^{L17} may be bonded to each other to form a ring having 3 to 20 carbon atoms together with the carbon atom to which they are bonded. The ring is preferably a ring having 4 to 16 carbon atoms, and particularly preferably an alicyclic ring.

Specific examples of the repeating unit-a1 include, but are not limited to, those shown below. Note that, in the following formulae, R^{A} and ALL is as defined above.

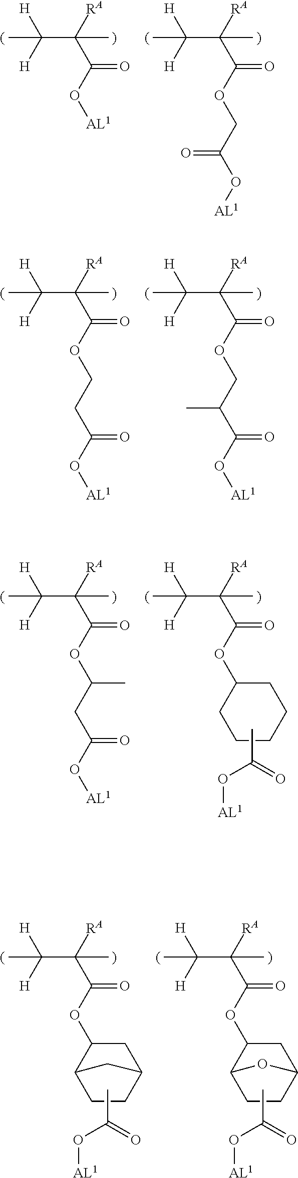

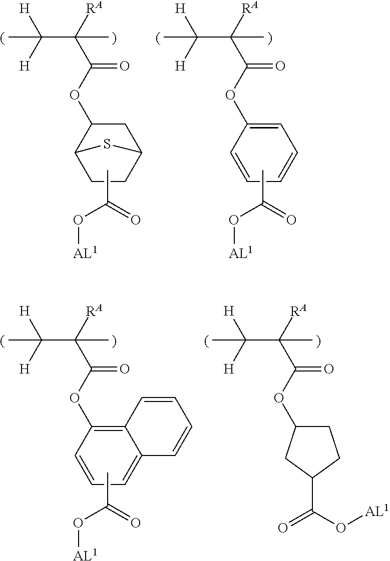

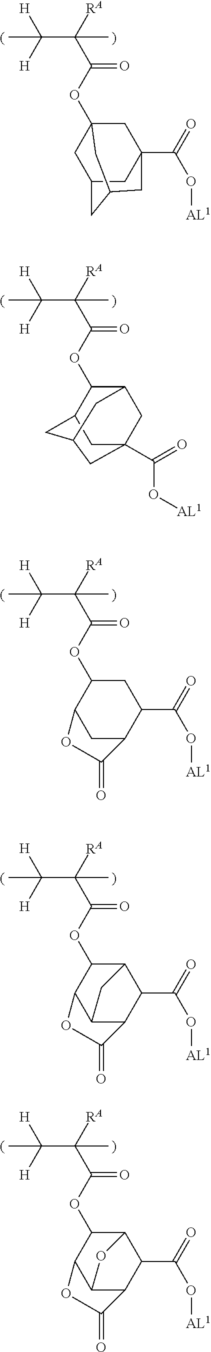

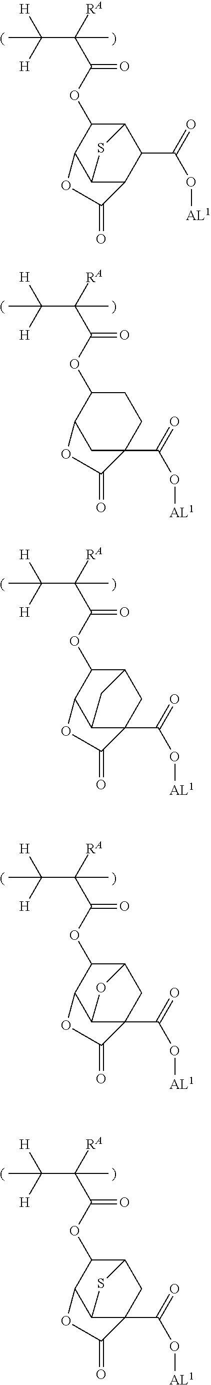

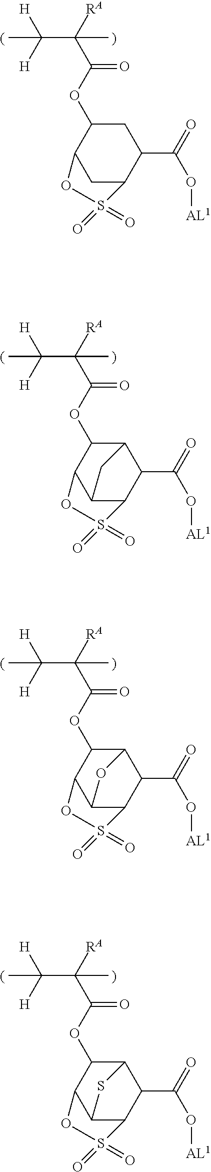

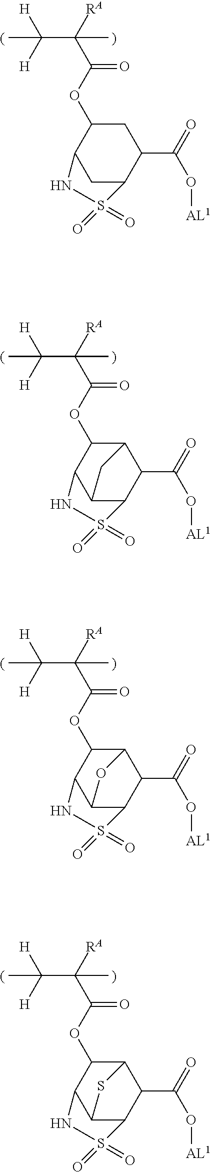

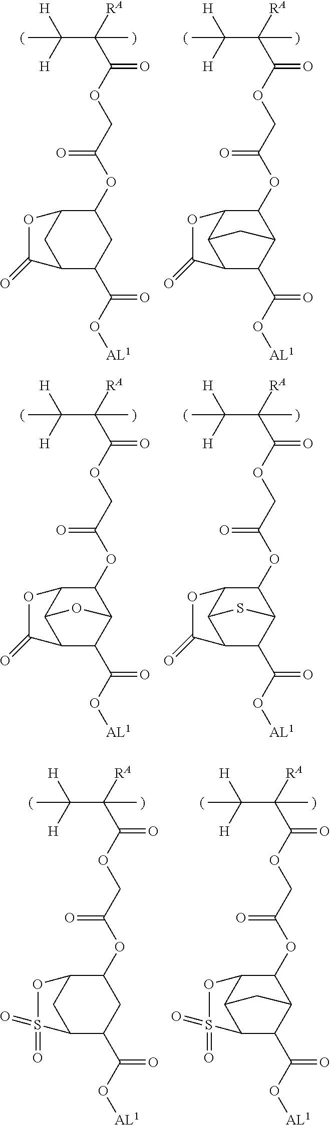

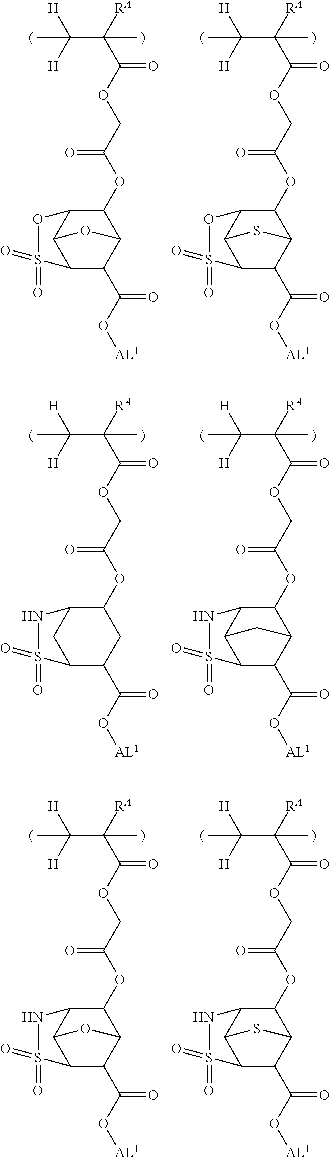

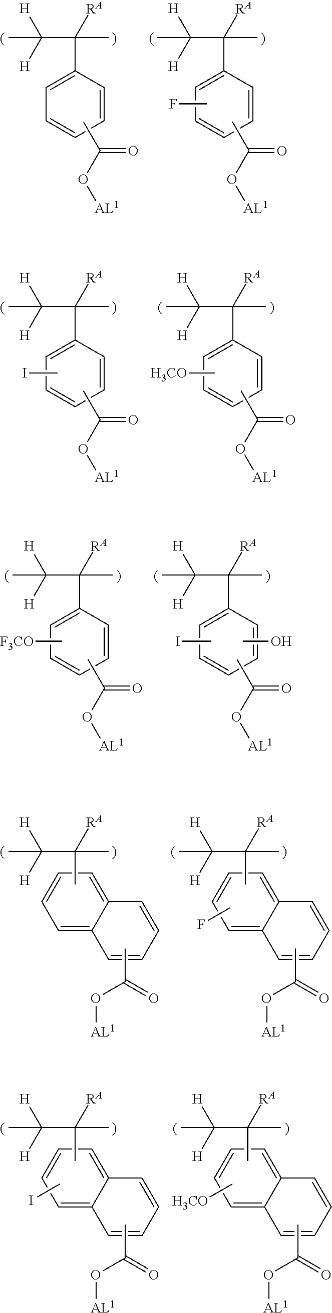

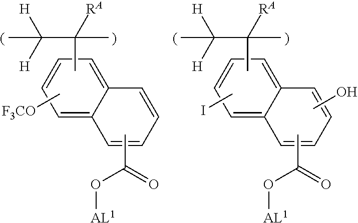

Specific examples of the repeating unit-a2 include, but are not limited to, those shown below. Note that, in the following formulae, R^{A} and A^{L2} are as defined above.

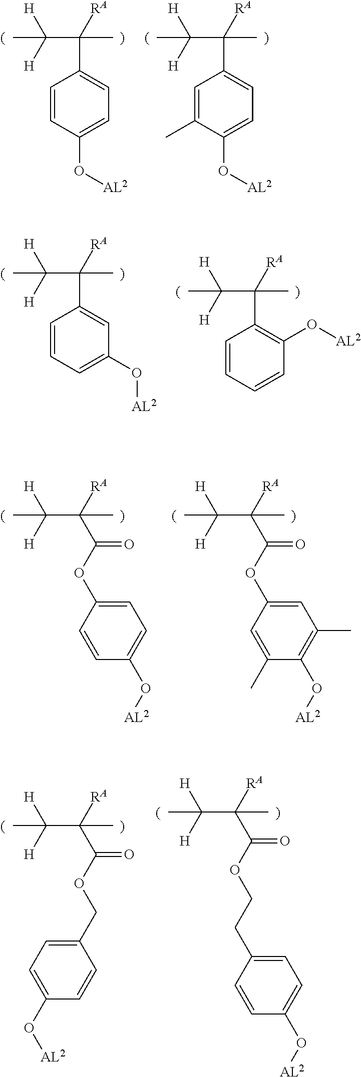

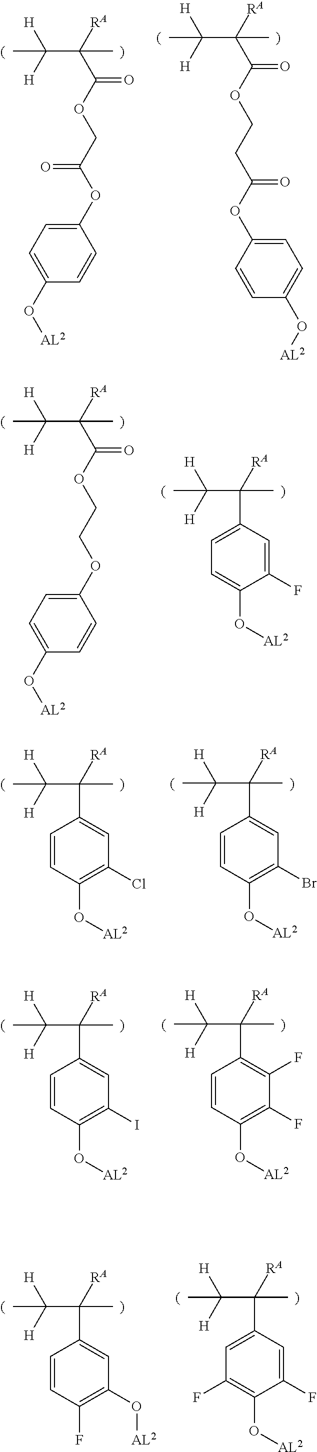

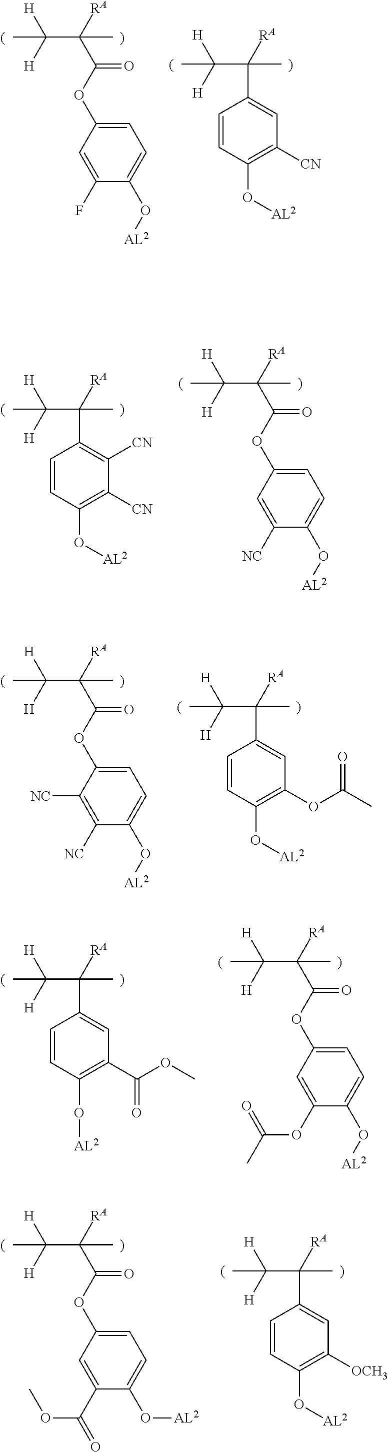

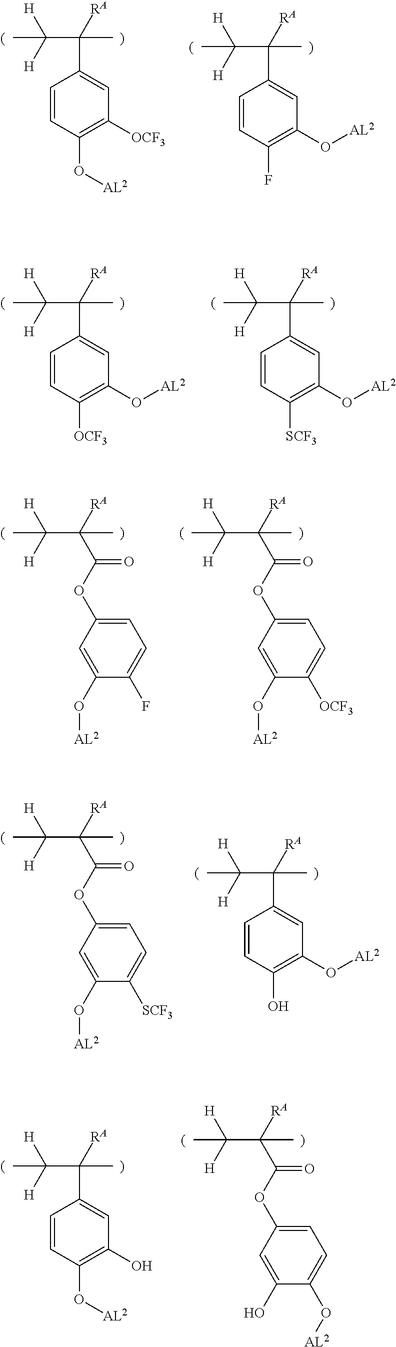

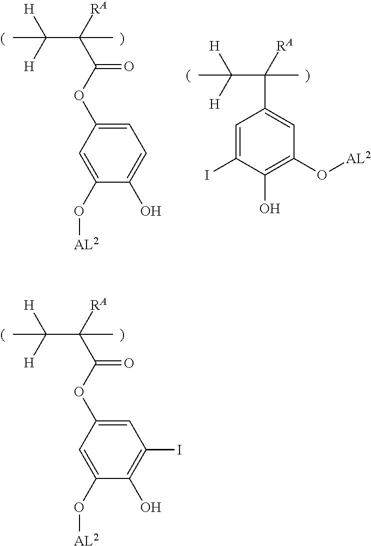

The base polymer (B) may also contain a repeating unit represented by the following general formula (a3) (hereinafter also referred to as repeating unit-a3).

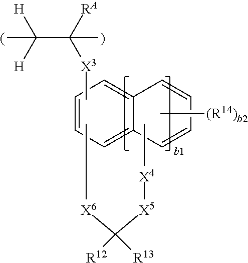

In the formula, “b1” is 0 or 1; “b2” is an integer from 0 to 3 when “b1” is 0, and an integer from 0 to 5 when “b1” is 1;
R^{A} is a hydrogen atom, a fluorine atom, a methyl group, or a trifluoromethyl group;
X³ is a single bond, *—C(═O)—O—, or *—C(═O)—NH—; represents a bond to a main chain carbon atom;
X⁴ is a single bond, an aliphatic hydrocarbylene group having 1 to 4 carbon atoms, a carbonyl group, a sulfonyl group, or a group obtained by combining these;
X⁵ and X⁶ are each independently an oxygen atom or a sulfur atom; provided that X⁴ and X⁶ are bonded to adjacent carbon atoms of an aromatic ring;
R¹² and R¹³ are each independently a hydrogen atom or a hydrocarbyl group having 1 to 20 carbon atoms and optionally containing a heteroatom; also, R¹² and R¹³ may be bonded to each other to form a ring together with the carbon atom to which they are bonded;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, or a hydrocarbylthio group having 1 to 20 carbon atoms and optionally containing a heteroatom, or —N(R^{14A})(R^{14B}); R^{14A} and R^{14B} are each independently a hydrogen atom or a hydrocarbyl group having 1 to 6 carbon atoms; and when “b2” is 2 or more, each R¹⁴ may be the same as or different from each other, and a plurality of R¹⁴ may be bonded to each other to form a ring together with the carbon atoms of the aromatic ring to which they are bonded.

In the general formula (a3), “b1” is 0 or 1. When “b1” is 0, the structure is a benzene ring, and when “b1” is 1, it is a naphthalene ring; however, from the viewpoint of solvent solubility, “b1” is preferably 0, giving a benzene ring. “b2” is an integer from 0 to 3 when “b1” is 0, and is an integer from 0 to 5 when “b1” is 1. From the viewpoint of raw material procurement, “b2” is preferably an integer from 0 to 3, and more preferably 0, 1, or 2.

In the general formula (a3), R^{A} is a hydrogen atom, a fluorine atom, a methyl group, or a trifluoromethyl group. Among these, a hydrogen atom or a methyl group is preferable, and a hydrogen atom is more preferable.

In the general formula (a3), X³ is a single bond, *—C(═O)—O—, or *—C(═O)—NH—. “*” represents a bond to a main chain carbon atom. Among these, a single bond or *—C(═O)—O— is preferable, and a single bond is more preferable.

In the general formula (a3), X⁴ is a single bond, an aliphatic hydrocarbylene group having 1 to 4 carbon atoms, a carbonyl group, a sulfonyl group, or a group obtained by combining these. Among these, from the viewpoint of raw material procurement, a single bond, a carbonyl group, or a sulfonyl group is preferable, and from the viewpoint of a polar group generated after the reaction, a single bond or a carbonyl group is more preferable.

In the general formula (a3), X⁵ and X⁶ are each independently an oxygen atom or a sulfur atom. Provided that X⁴ and X⁶ are bonded to adjacent carbon atoms of an aromatic ring. X⁵ and X⁶ may be the same as or different from each other, but from the viewpoint of reactivity, X⁵ and X⁶ are preferably both oxygen atoms.

In the general formula (a3), R¹² and R¹³ are each independently a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; and groups obtained by combining these groups. In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In addition, R¹² and R¹³ may be bonded to each other to form a ring together with the carbon atom to which they are bonded. Specific examples of the ring formed in this case include a cyclopropane ring, a cyclobutane ring, a cyclopentane ring, a cyclohexane ring, a norbornane ring, and an adamantane ring. In addition, some or all of the hydrogen atoms in the ring may be substituted with groups containing heteroatoms such as an oxygen atom, a sulfur atom, a nitrogen atom, and a halogen atom, and some of the —CH₂— may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In the general formula (a3), R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}). R^{14A} and R^{14B} are each independently a hydrogen atom or a hydrocarbyl group having 1 to 6 carbon atoms. The halogen atom is preferably a fluorine atom, a chlorine atom, a bromine atom, or an iodine atom, and more preferably a fluorine atom or an iodine atom. The hydrocarbyl group and the hydrocarbyl moiety of the hydrocarbyloxy group, hydrocarbyloxycarbonyl group, and hydrocarbylthio group may be saturated or unsaturated, and may be linear, branched, or cyclic. Specific examples thereof include the same ones as those given as examples of the hydrocarbyl group represented by R¹² and R¹³. In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. When “b2” is 2 or more, each R¹⁴ may be the same as or different from each other.

In addition, when “b2” is 2 or more, a plurality of R¹⁴ may be bonded to each other to form a ring together with the carbon atoms of the aromatic ring to which they are bonded. Specific examples of the ring formed in this case include a cyclopropane ring, a cyclobutane ring, a cyclopentane ring, a cyclohexane ring, a norbornane ring, and an adamantane ring. In addition, some or all of the hydrogen atoms in the ring may be substituted with groups containing heteroatoms such as an oxygen atom, a sulfur atom, a nitrogen atom, and a halogen atom, and some of the —CH₂— may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

That is, the chemically amplified resist composition preferably further contains a base polymer containing at least one repeating unit selected from the group consisting of a repeating unit represented by the general formula (a1), a repeating unit represented by the general formula (a2), and a repeating unit represented by the general formula (a3).

Specific examples of the repeating unit-a3 include, but are not limited to, those shown below. Note that, in the following formulae, R^{A} is as defined above, and Me is a methyl group. In addition, the bonding positions of the various substituents on the aromatic rings may be interchanged.

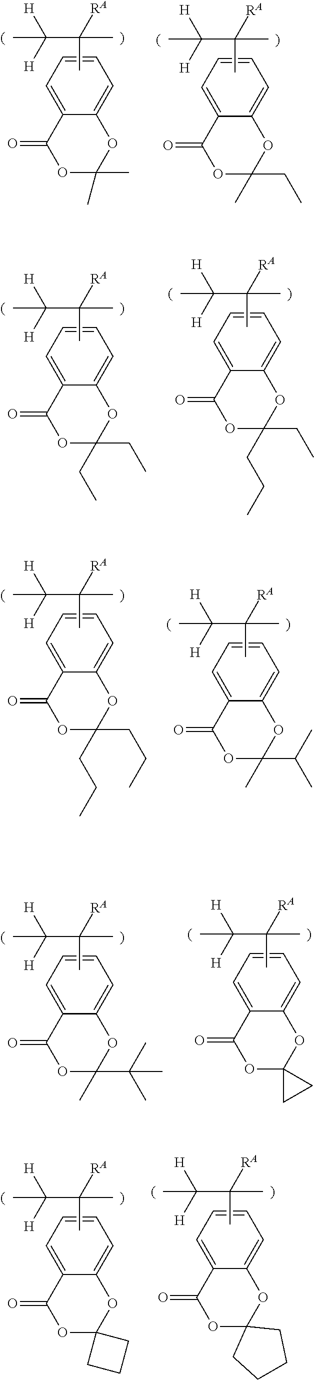

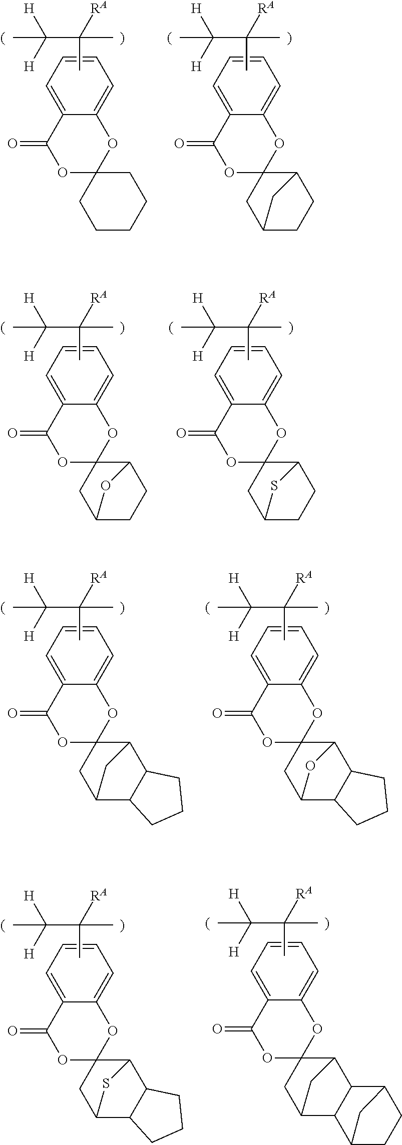

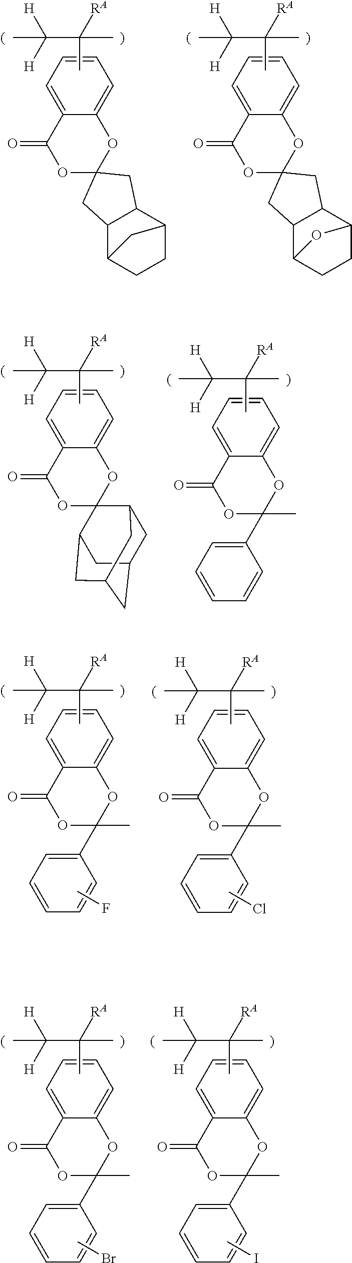

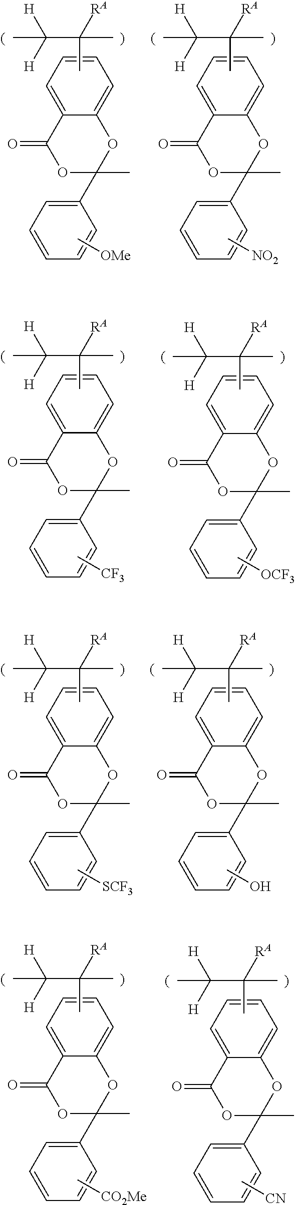

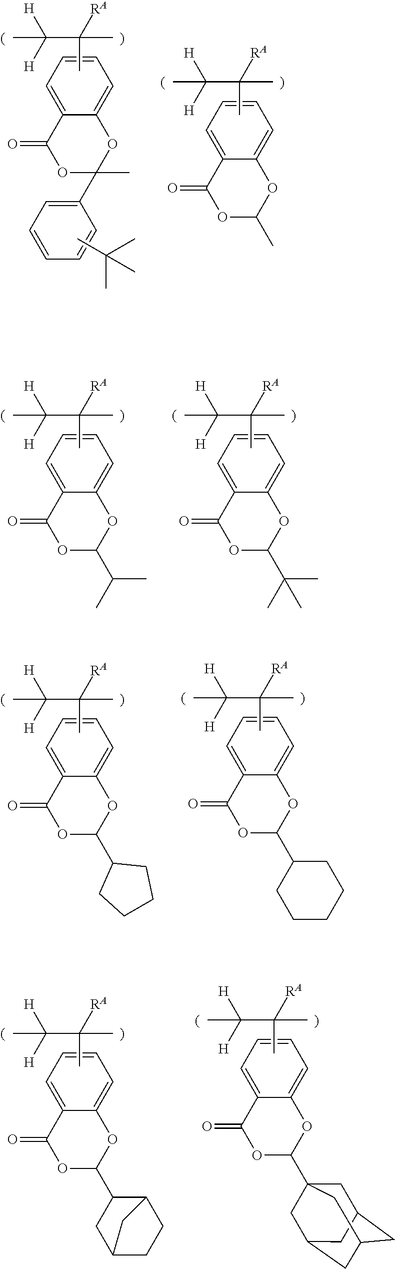

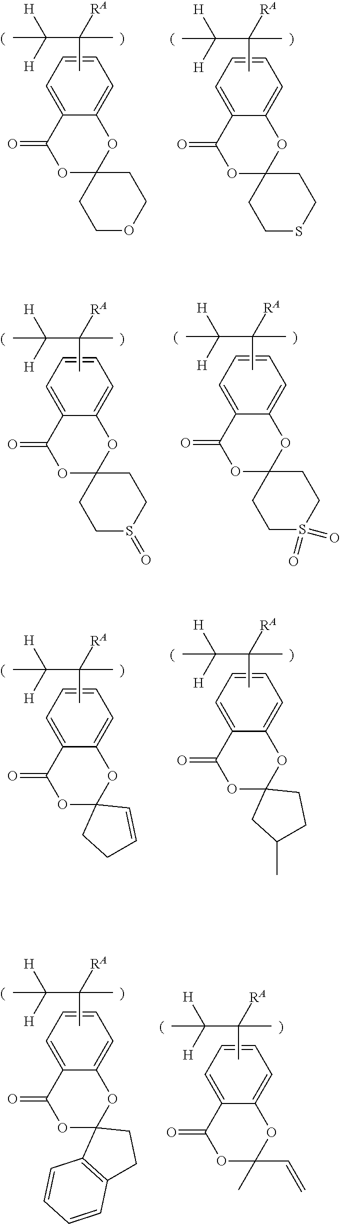

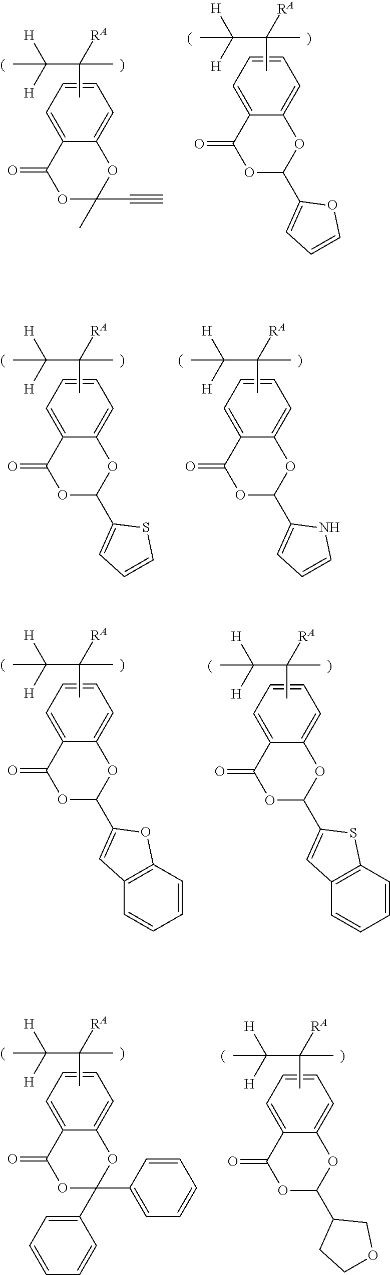

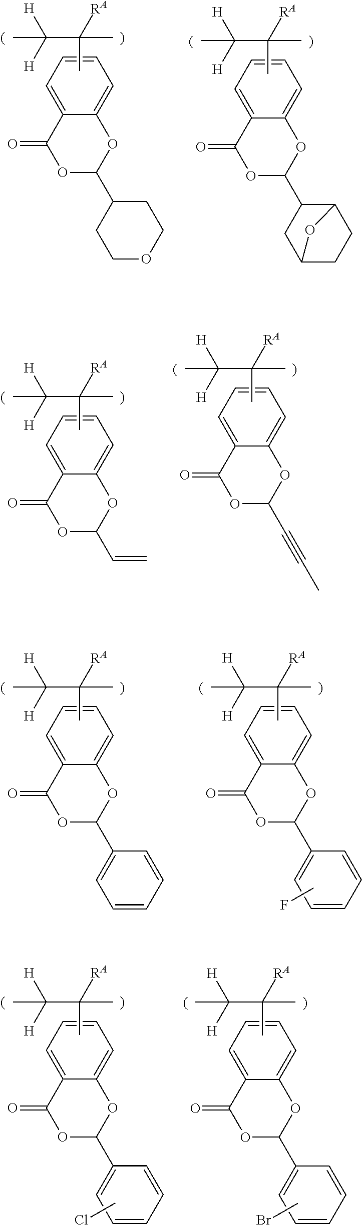

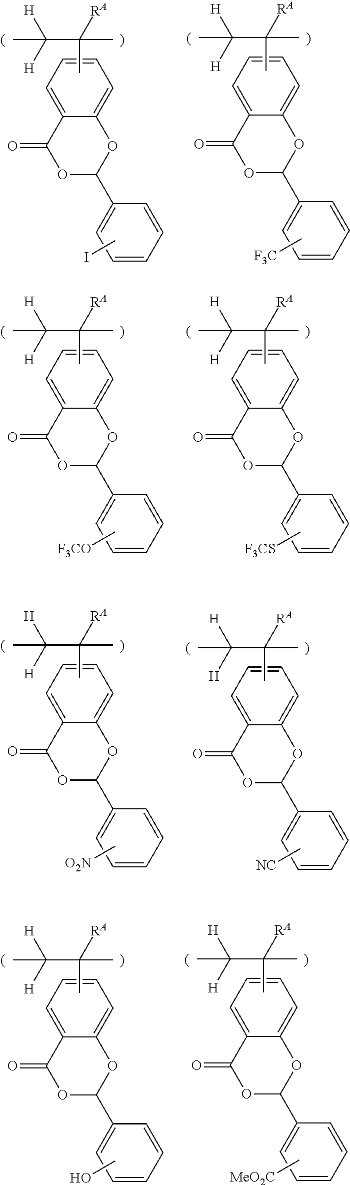

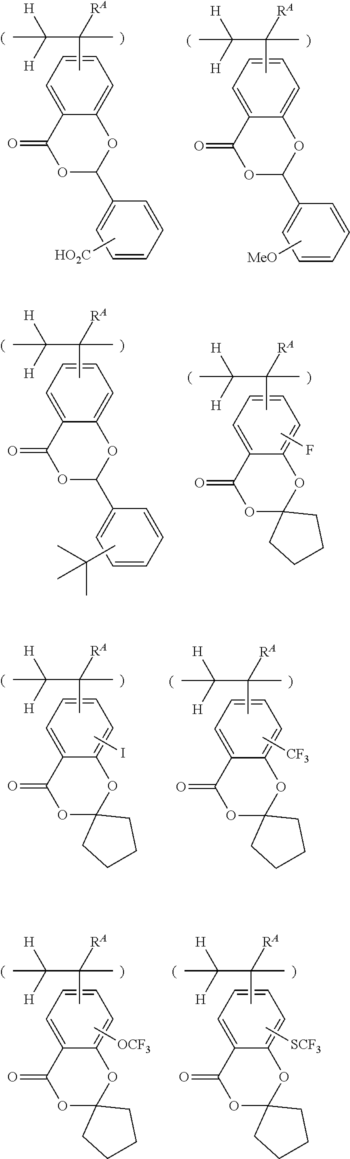

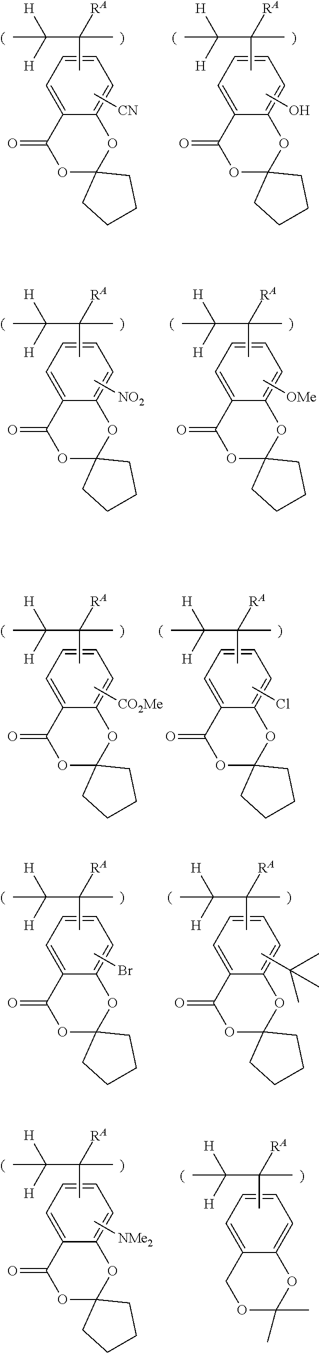

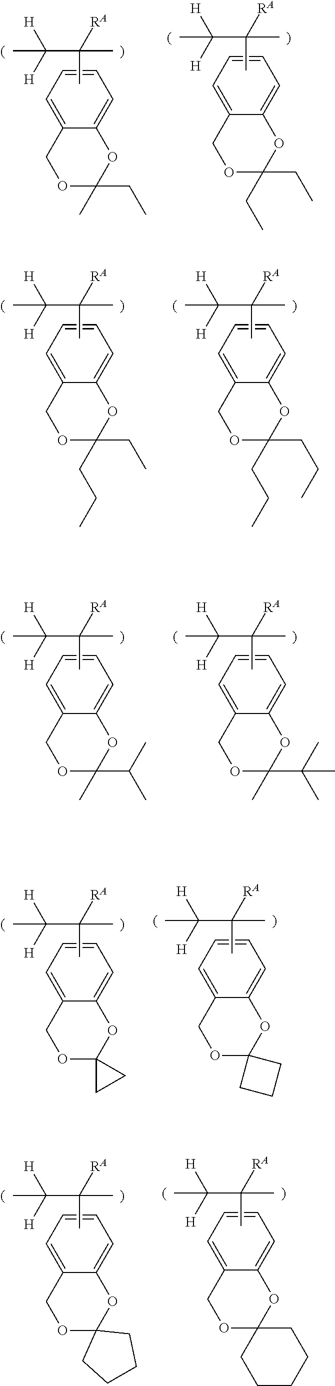

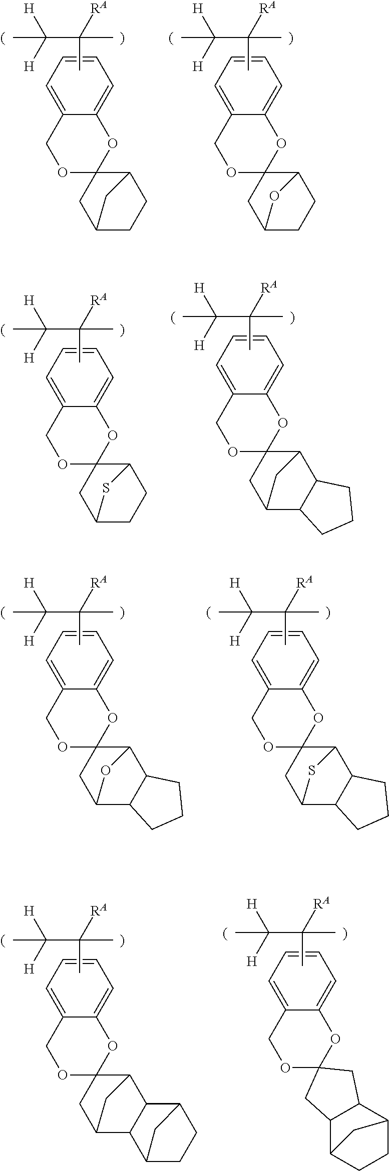

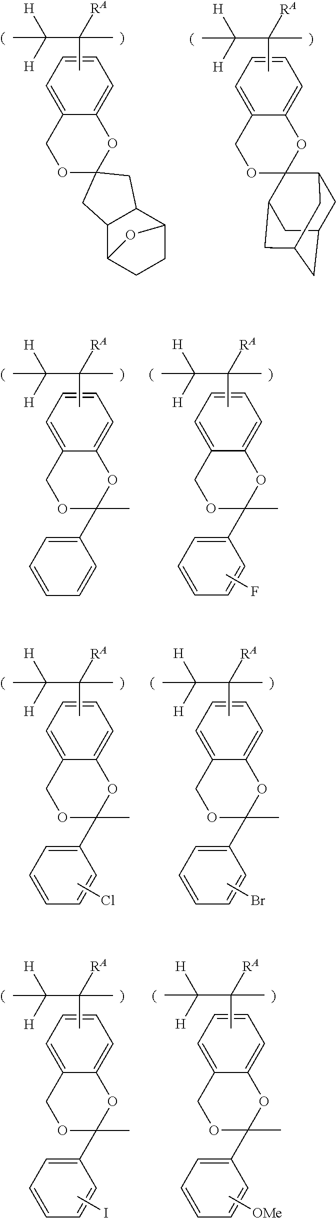

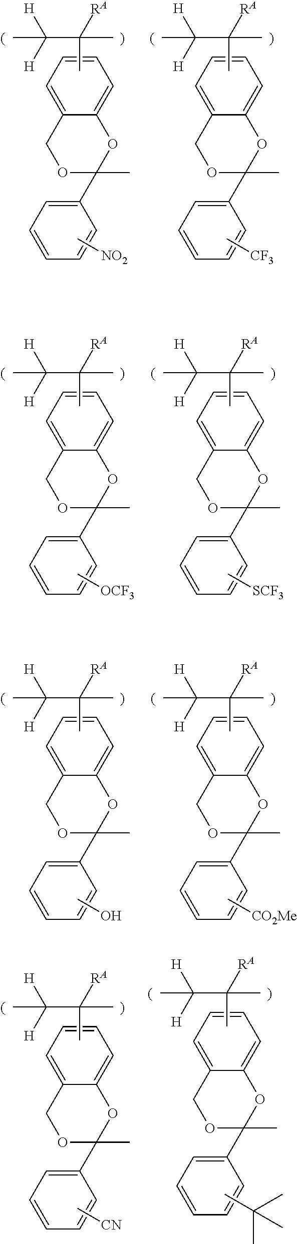

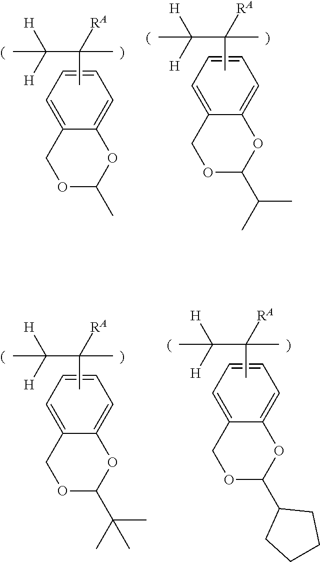

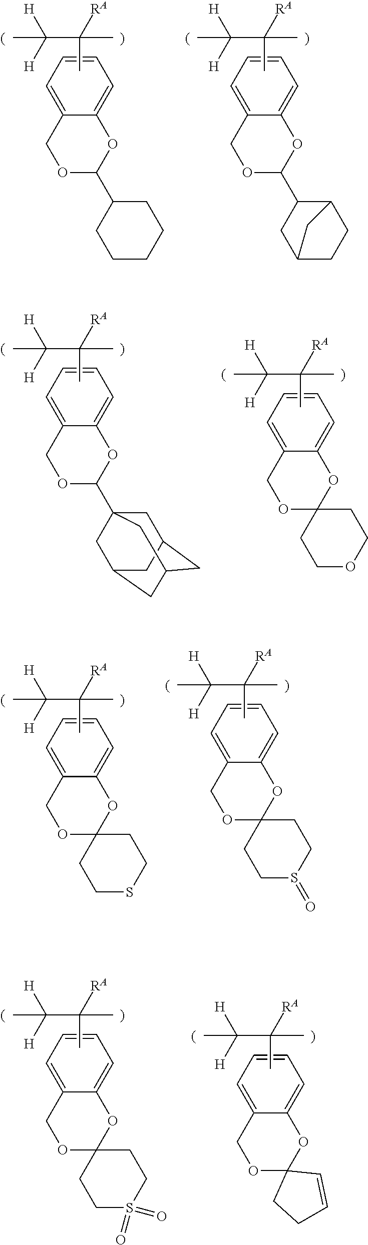

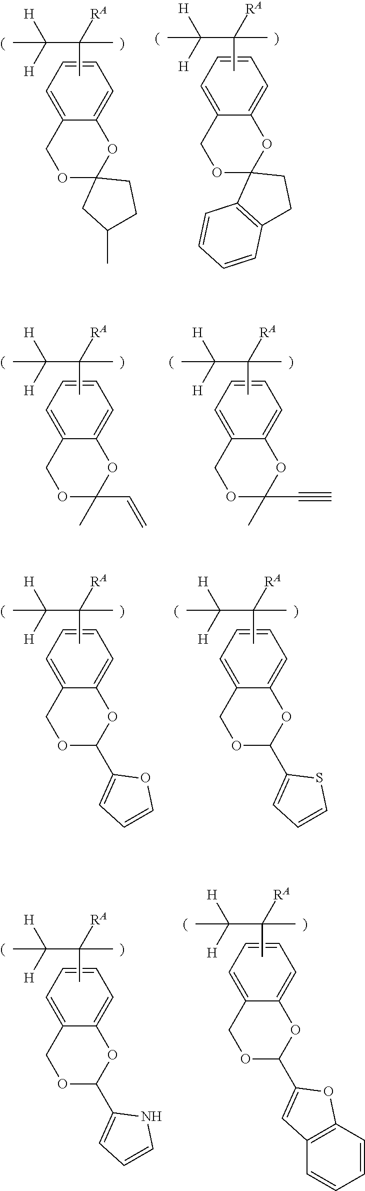

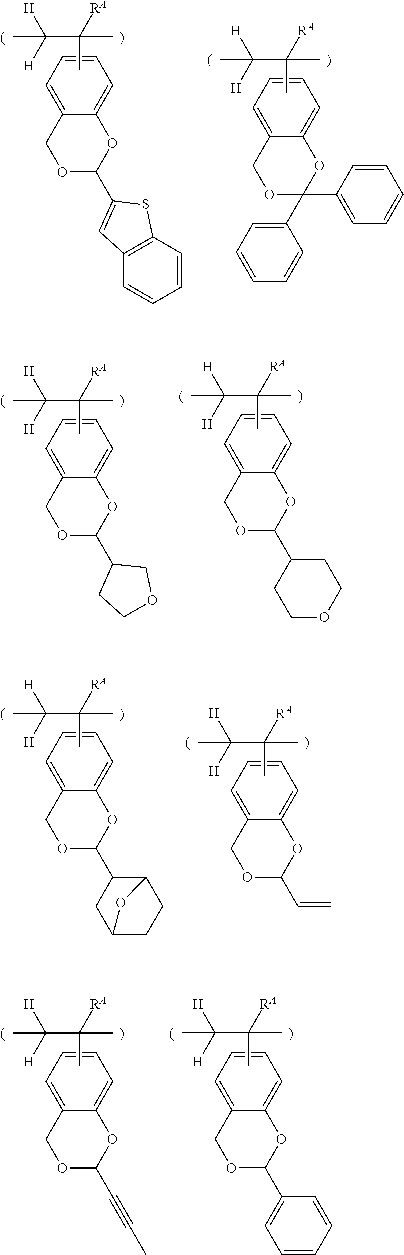

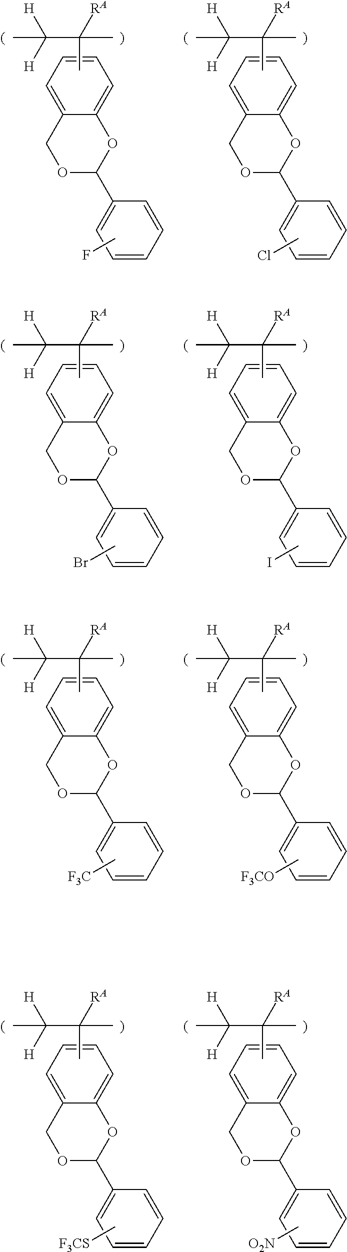

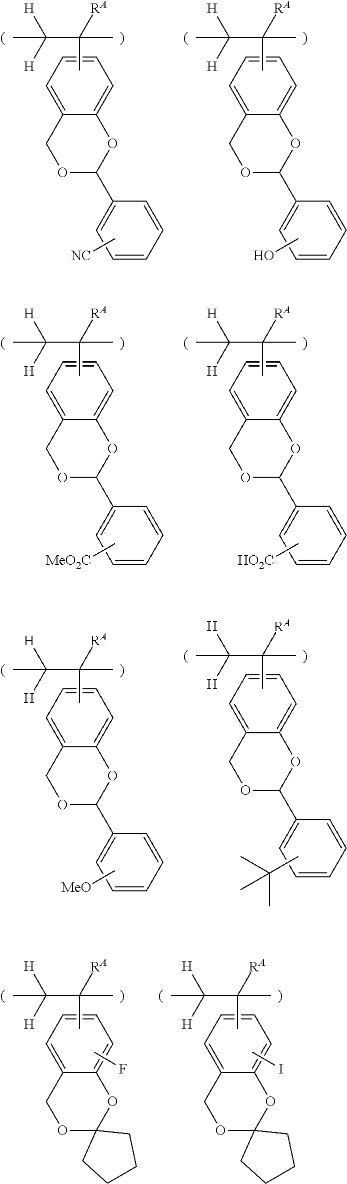

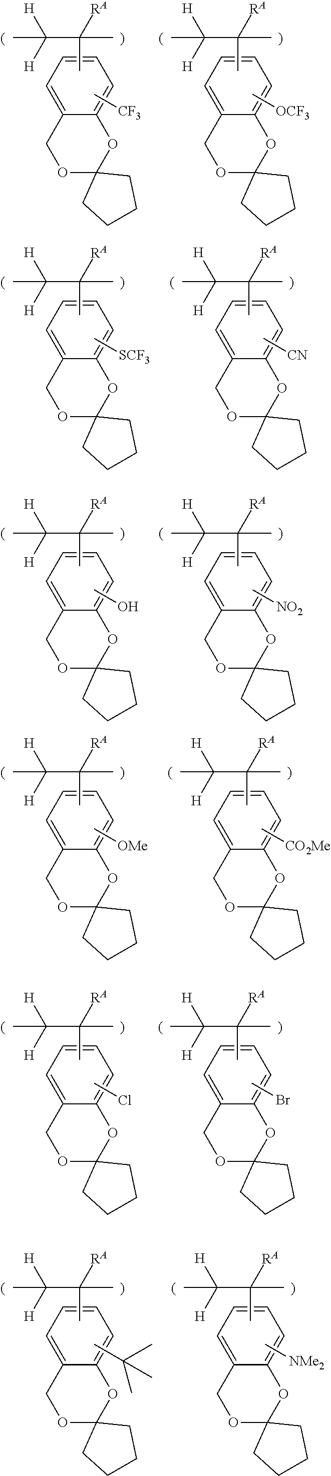

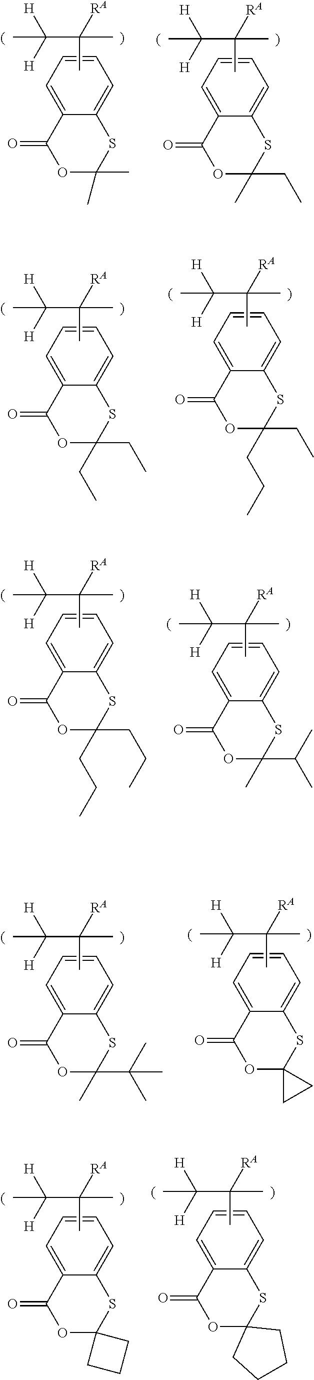

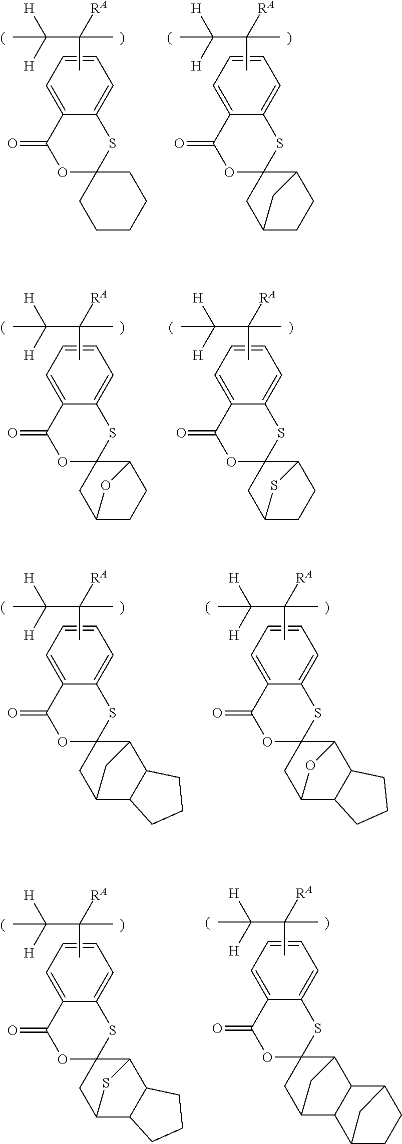

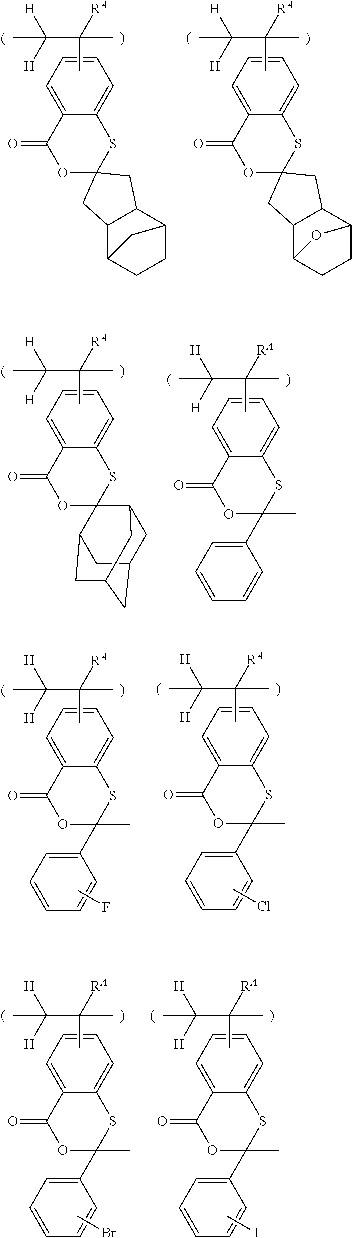

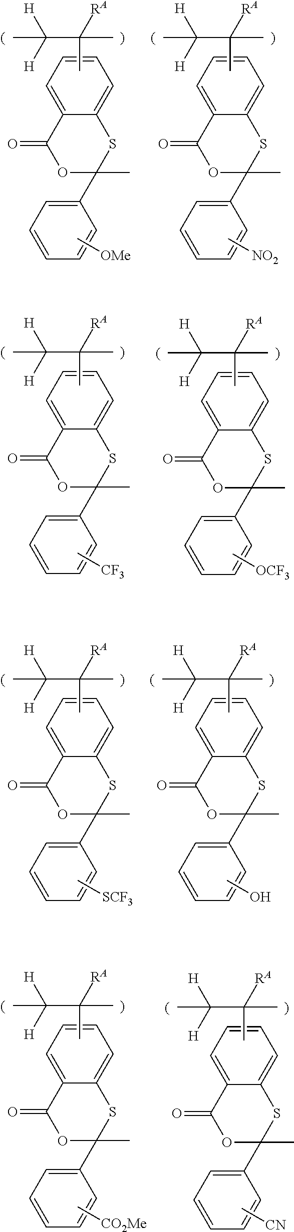

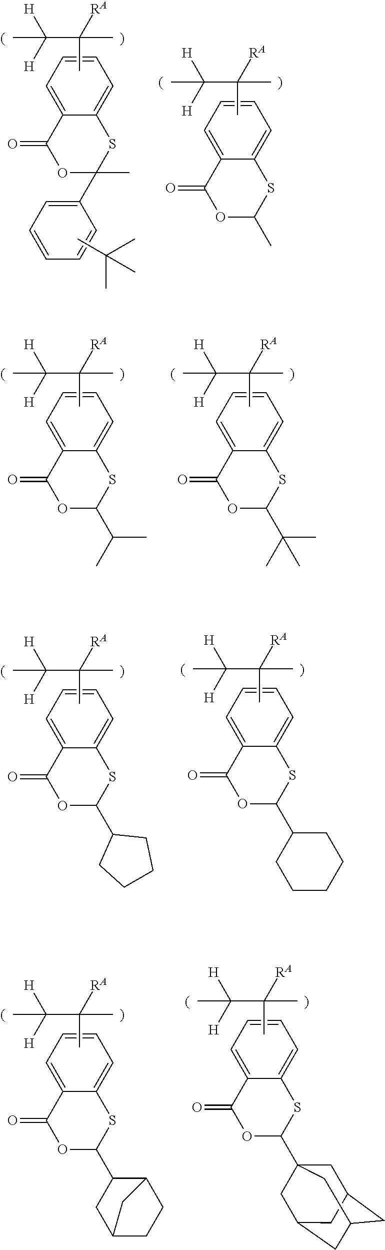

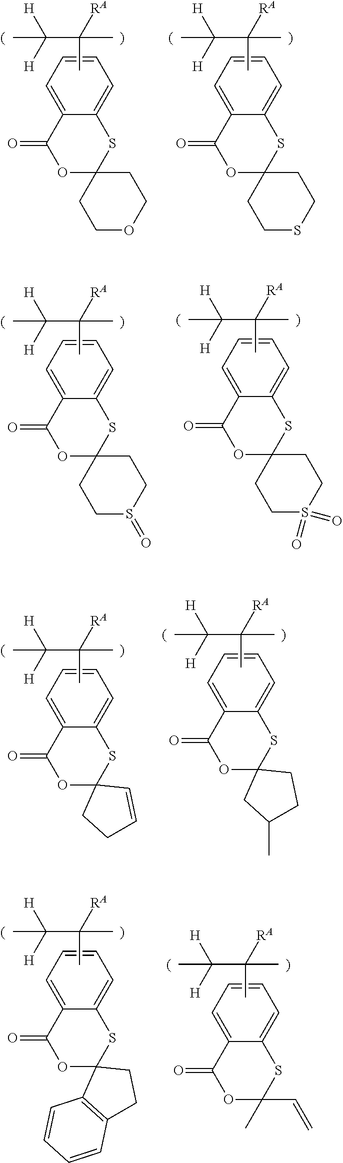

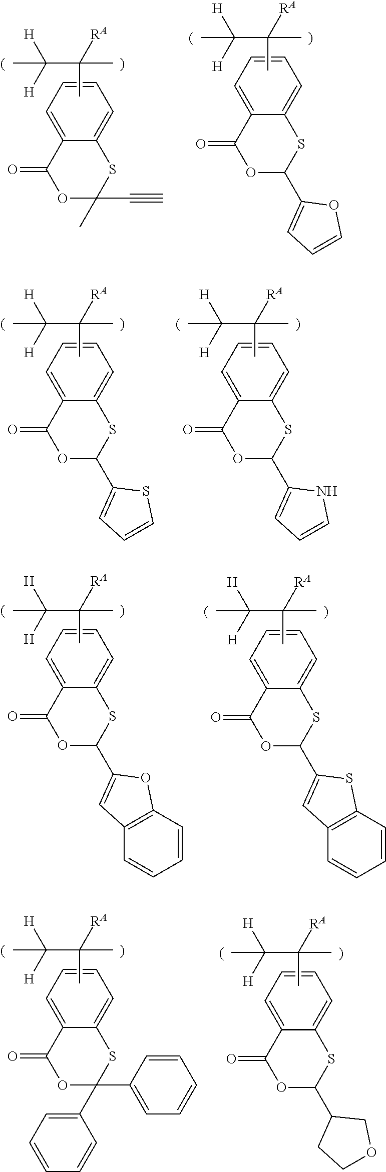

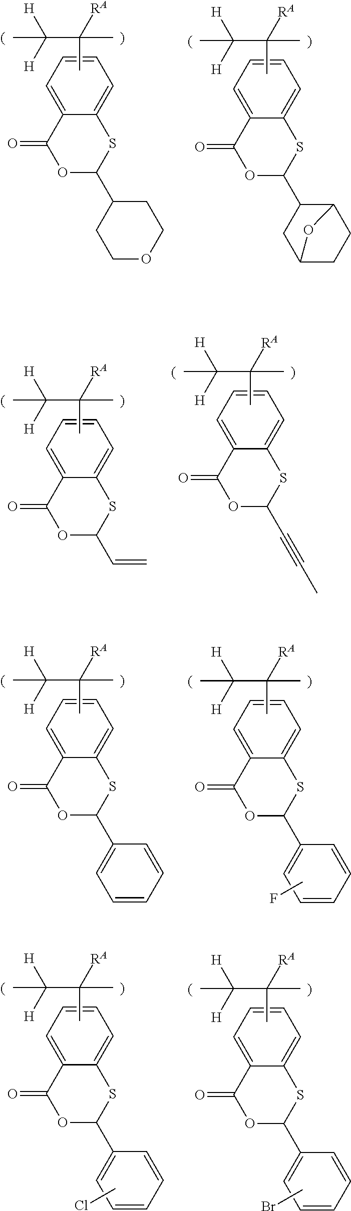

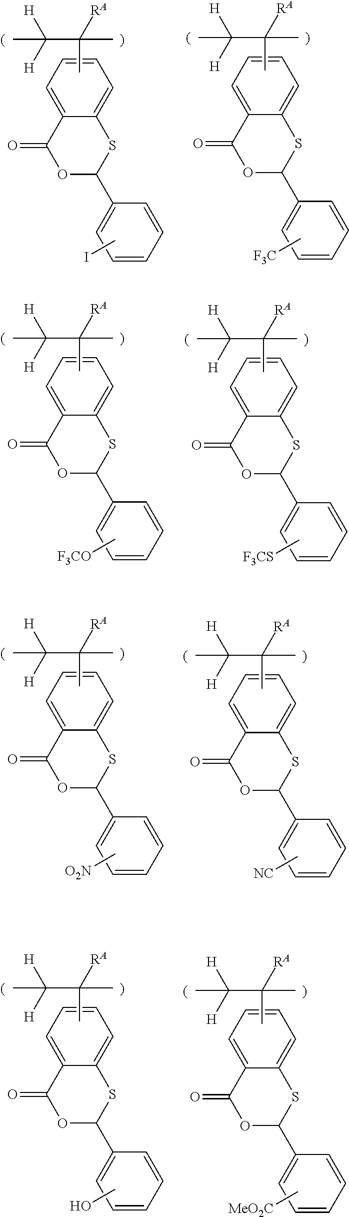

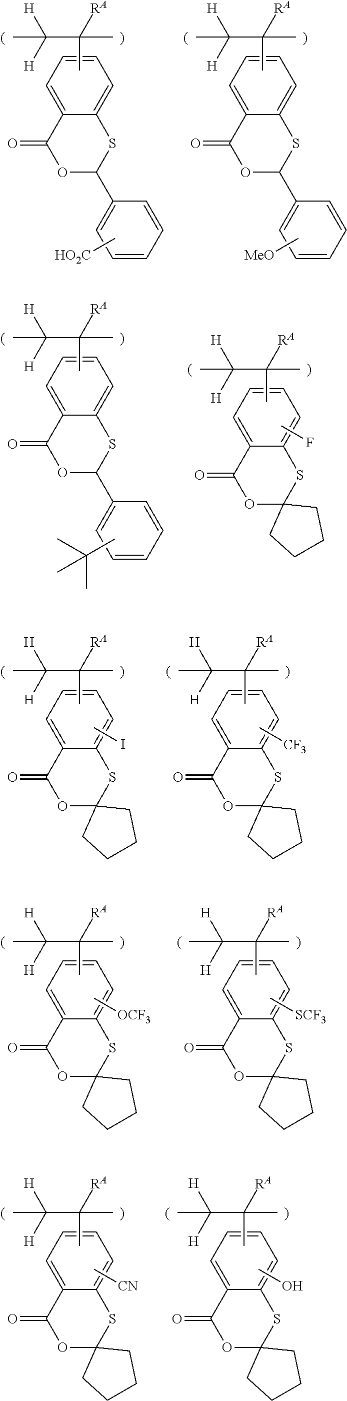

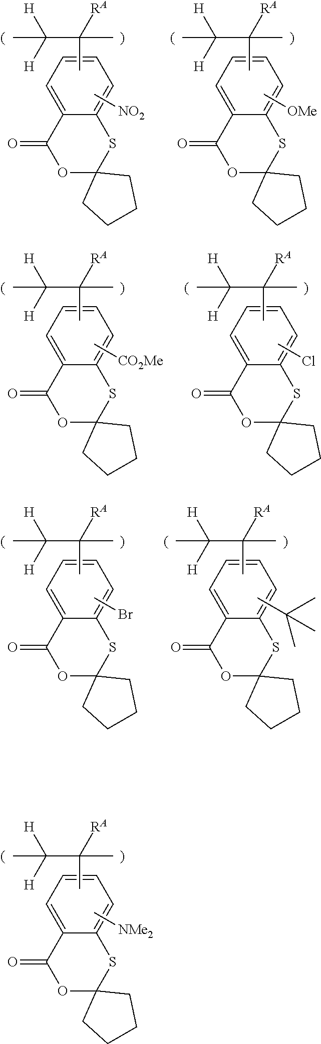

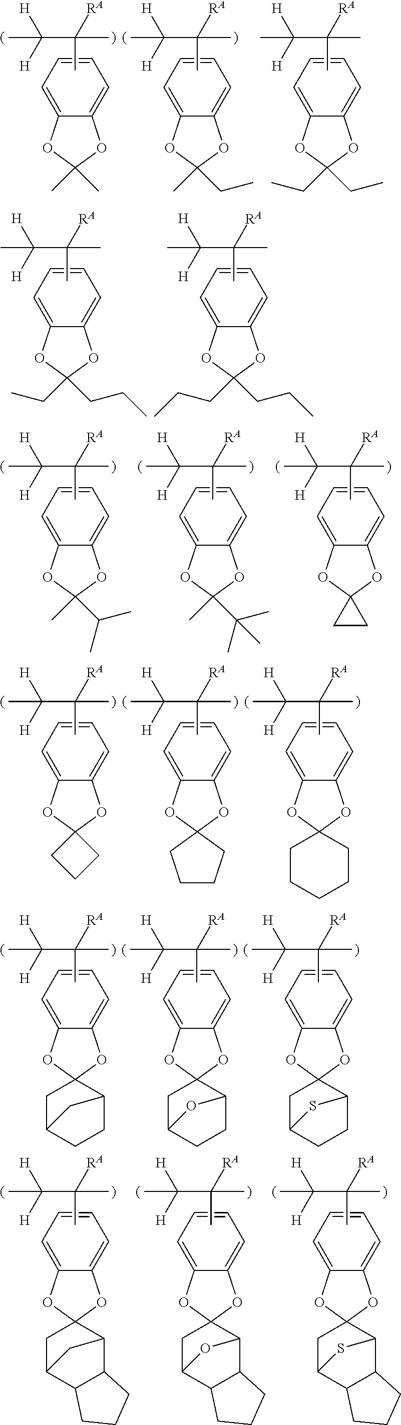

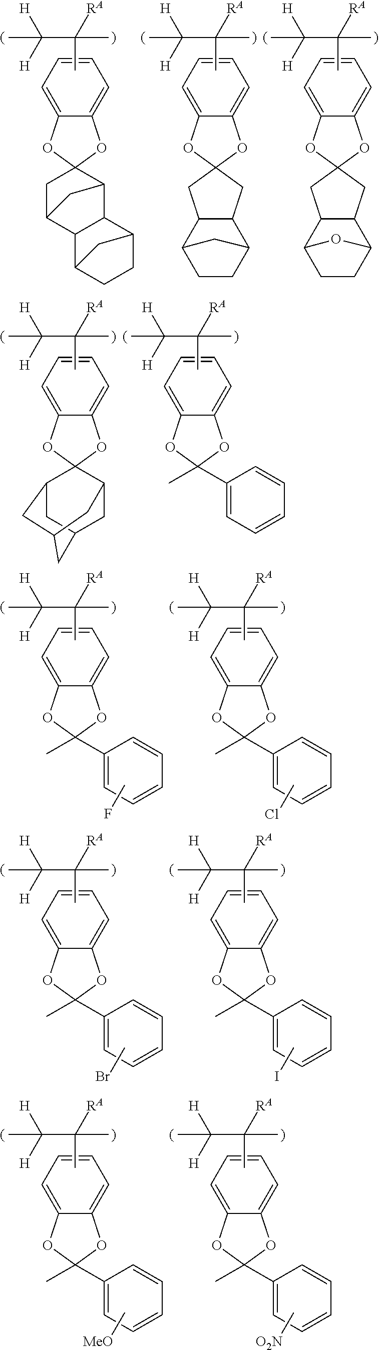

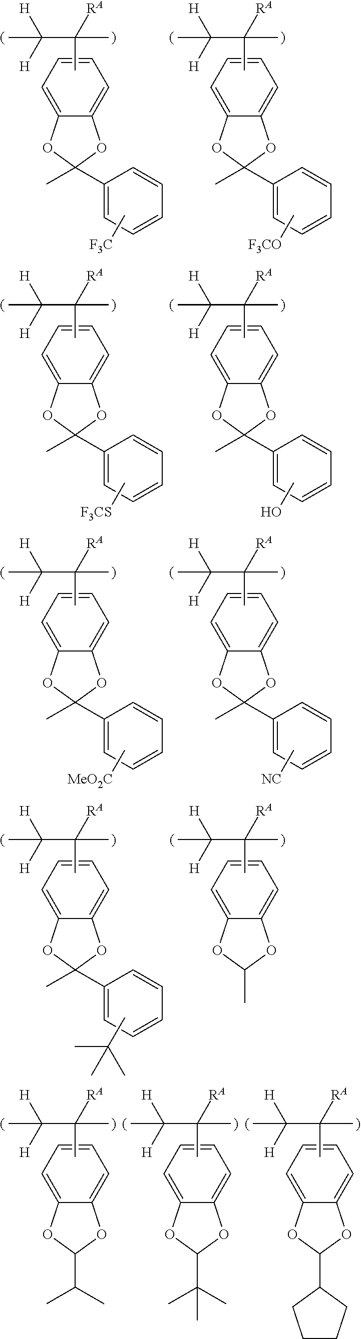

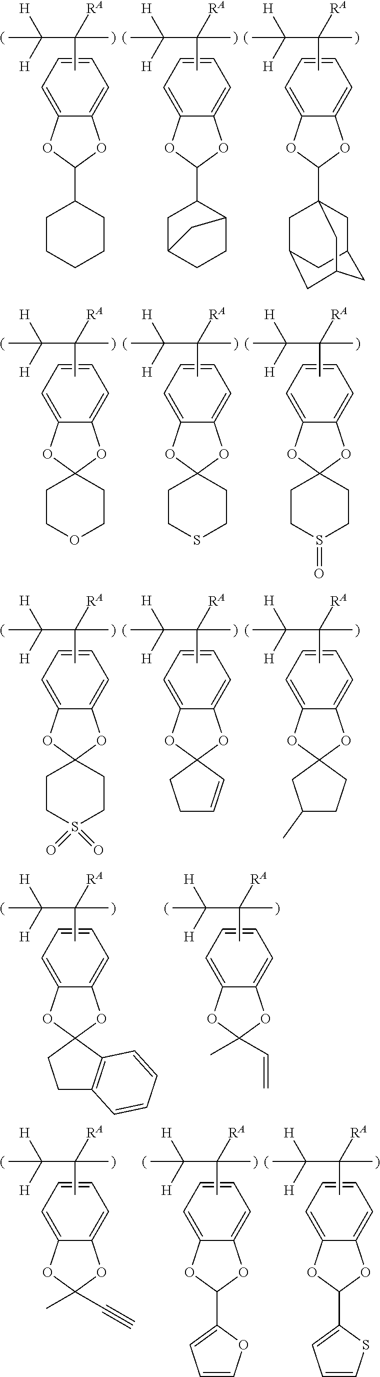

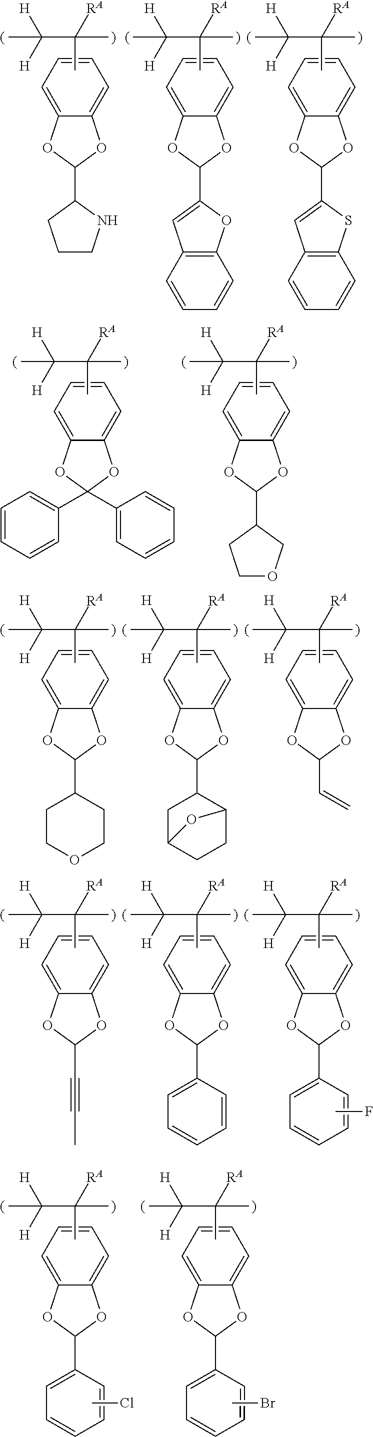

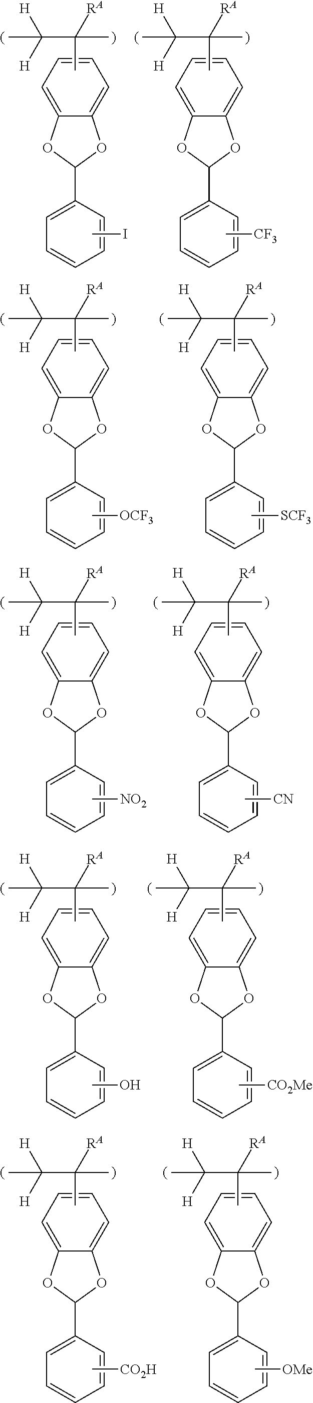

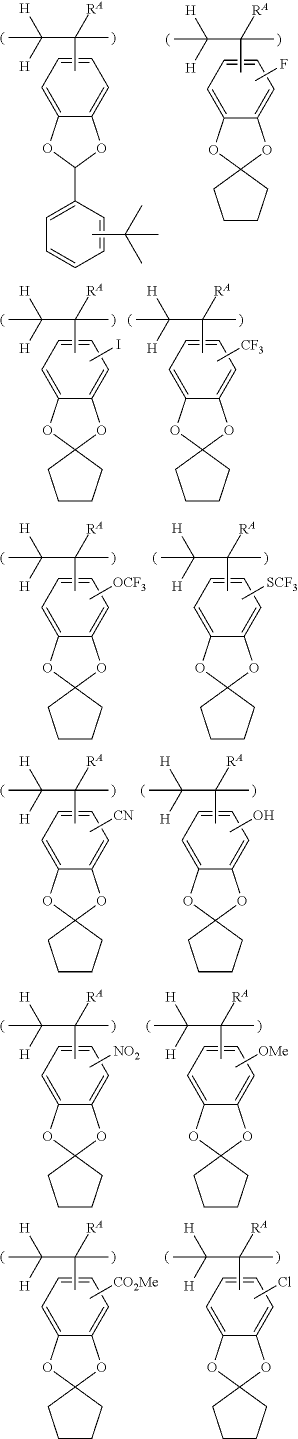

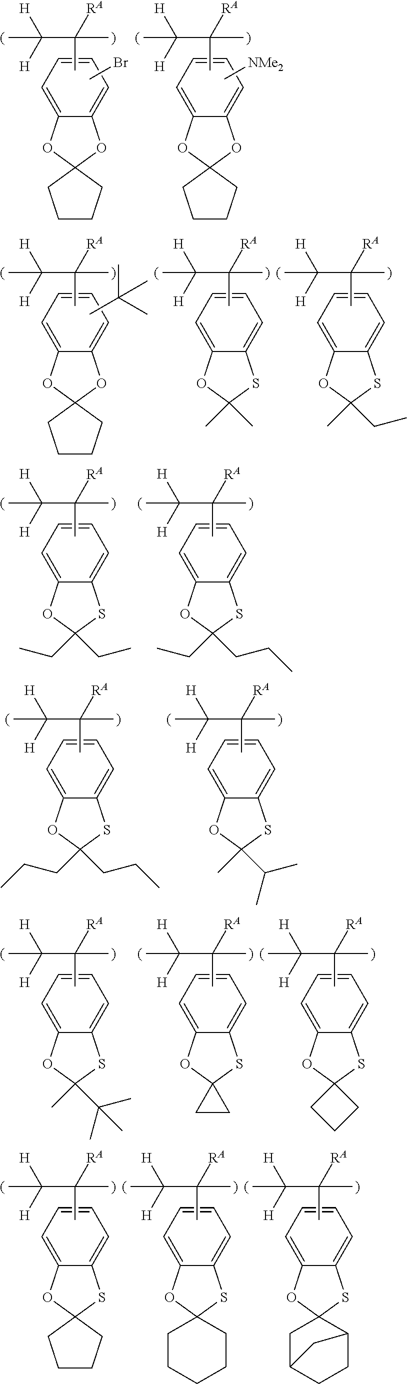

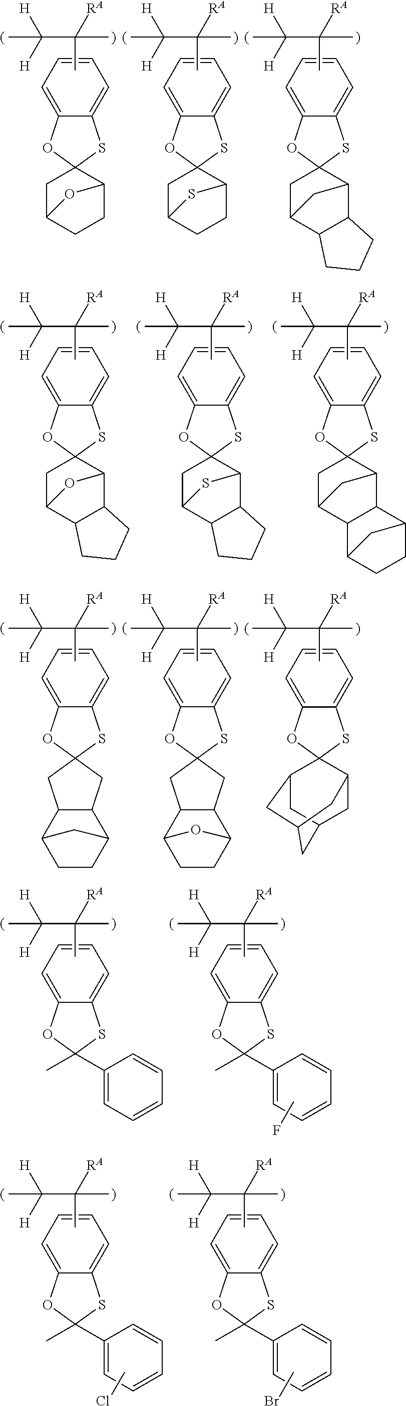

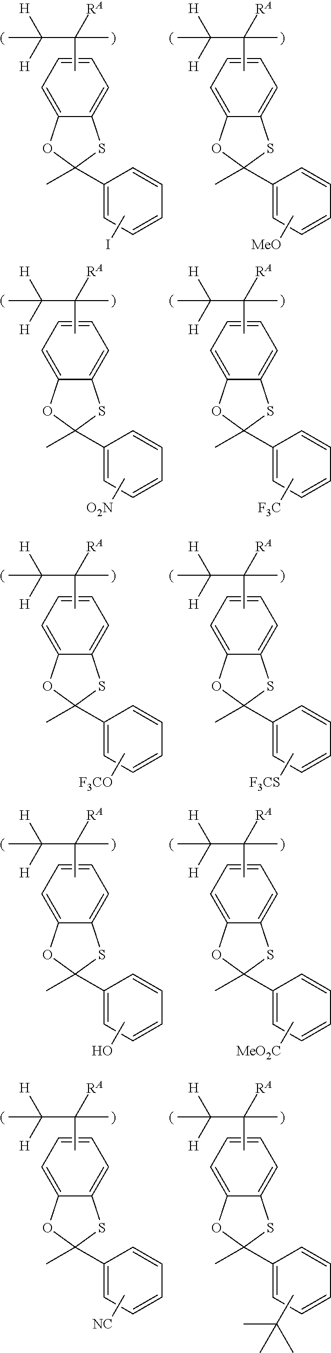

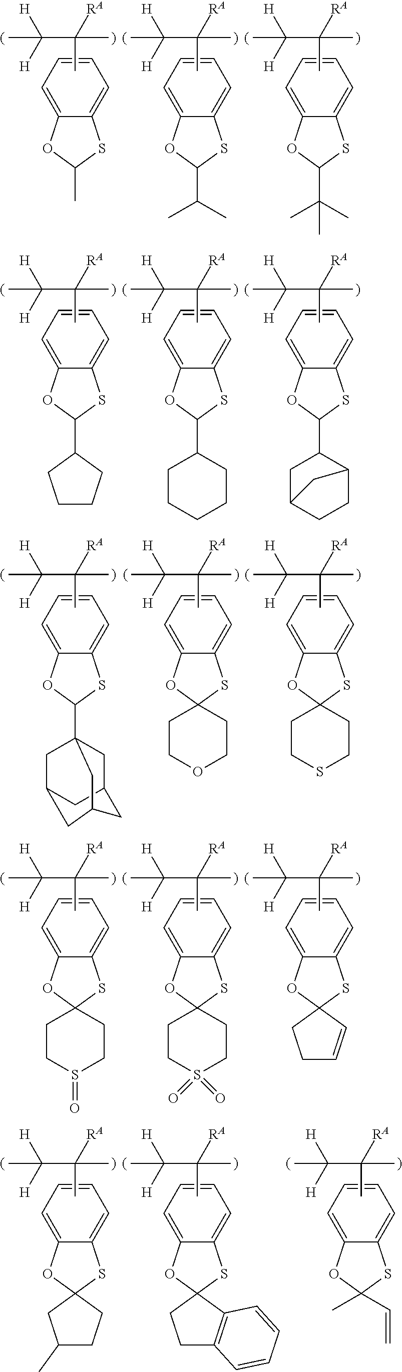

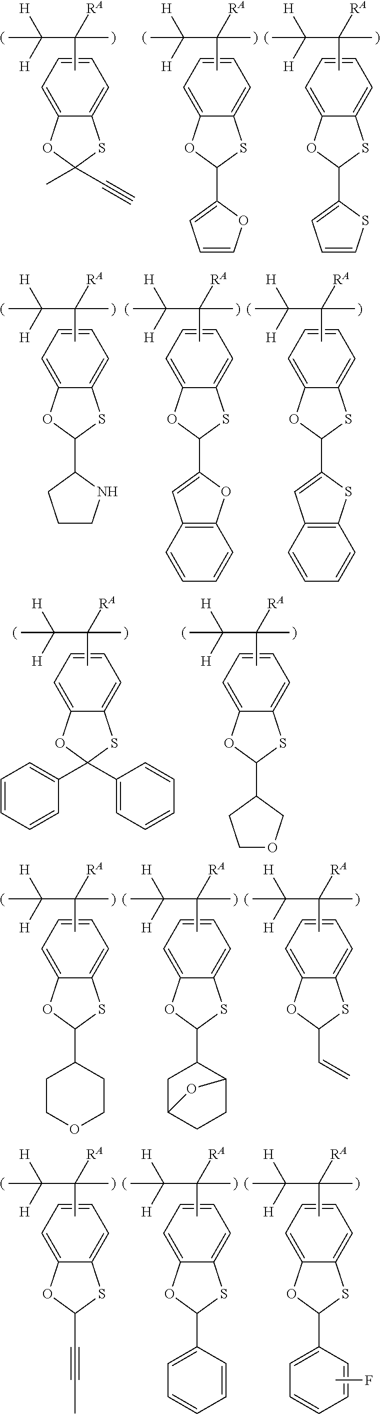

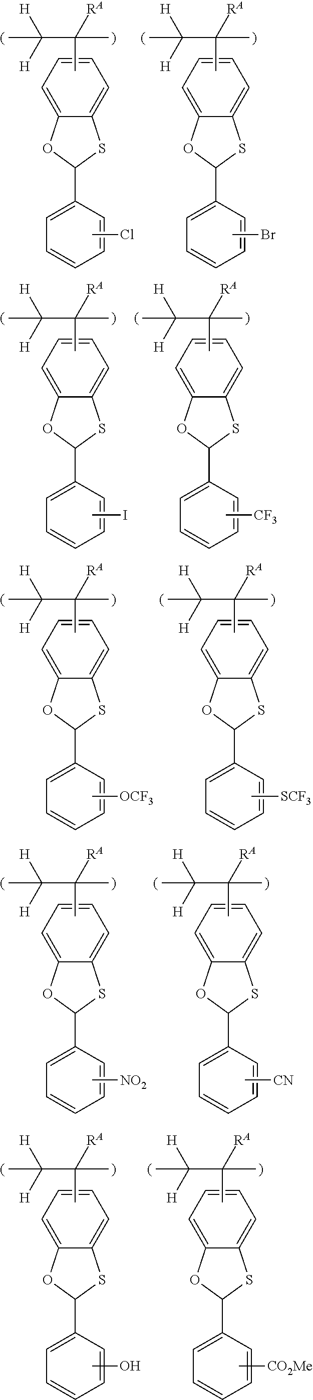

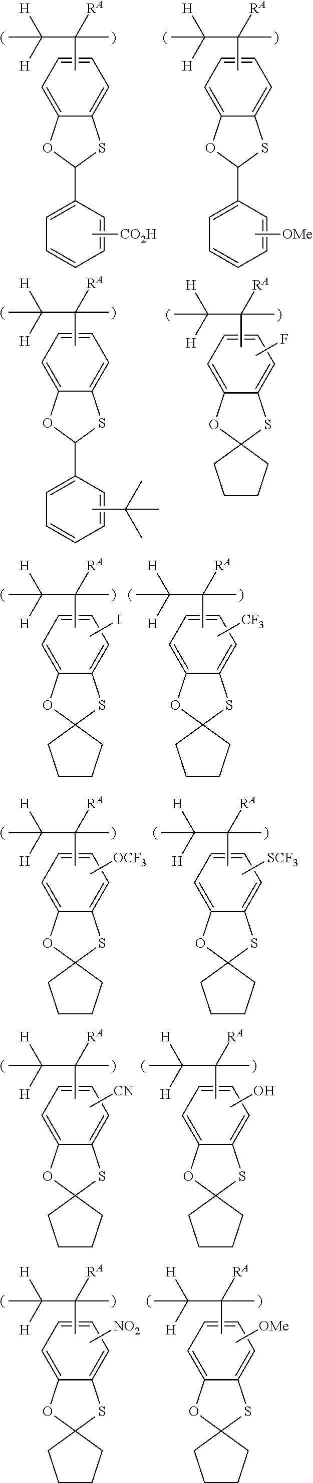

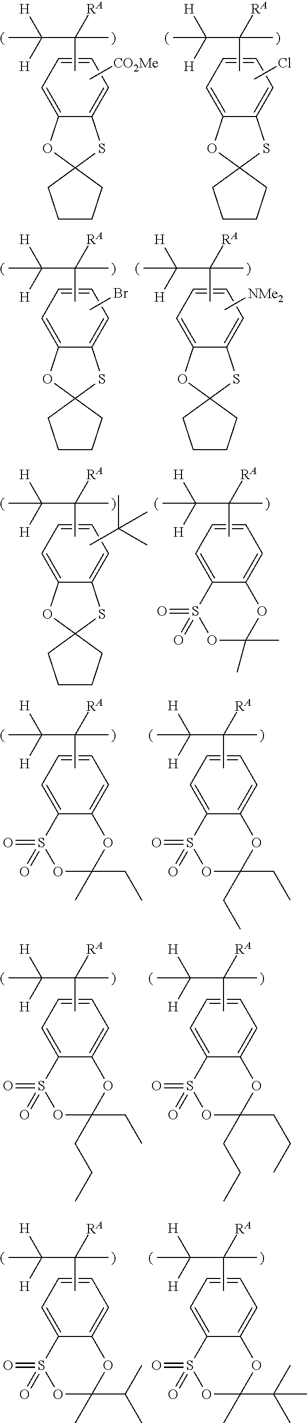

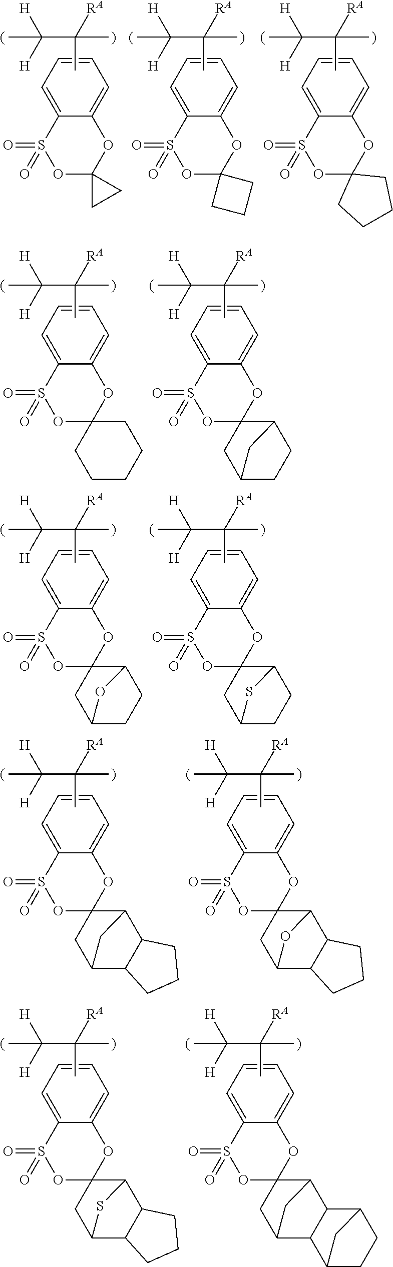

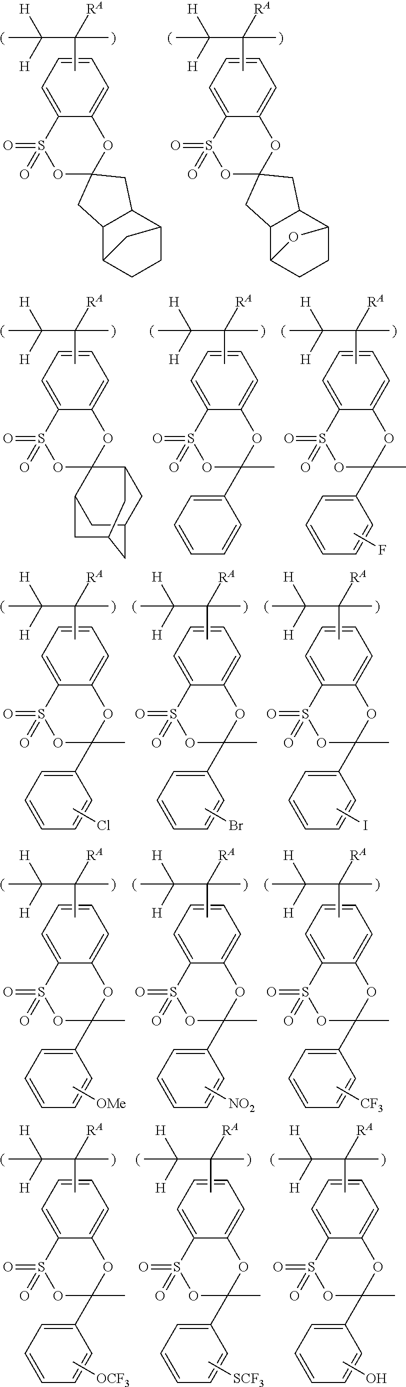

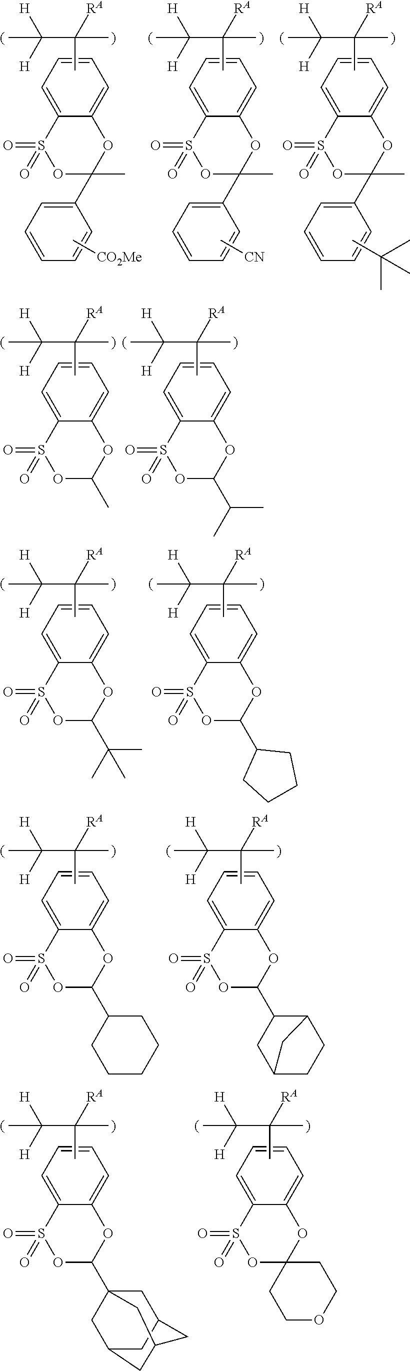

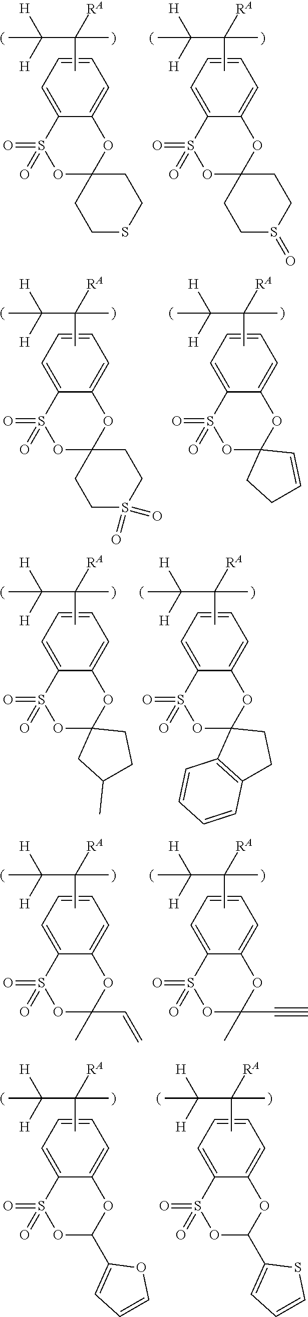

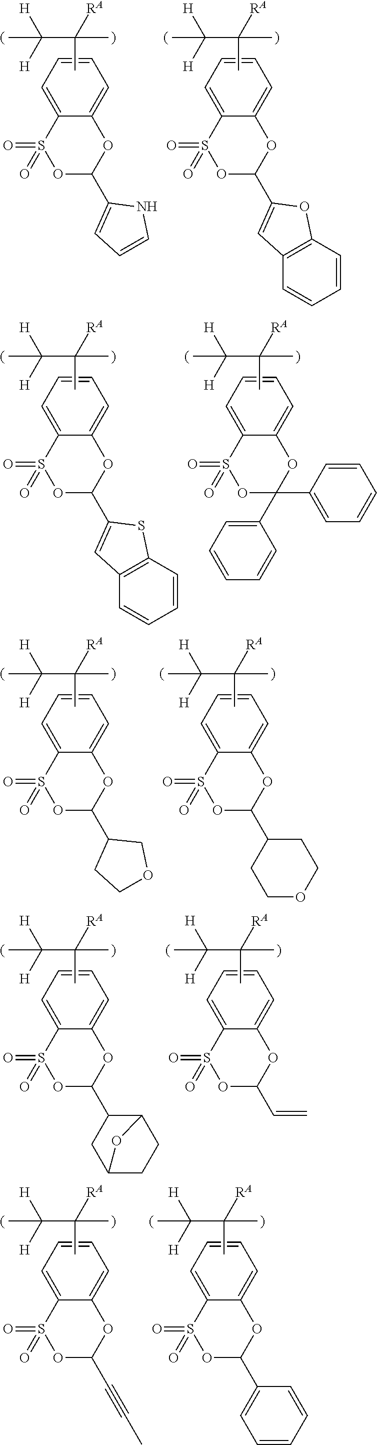

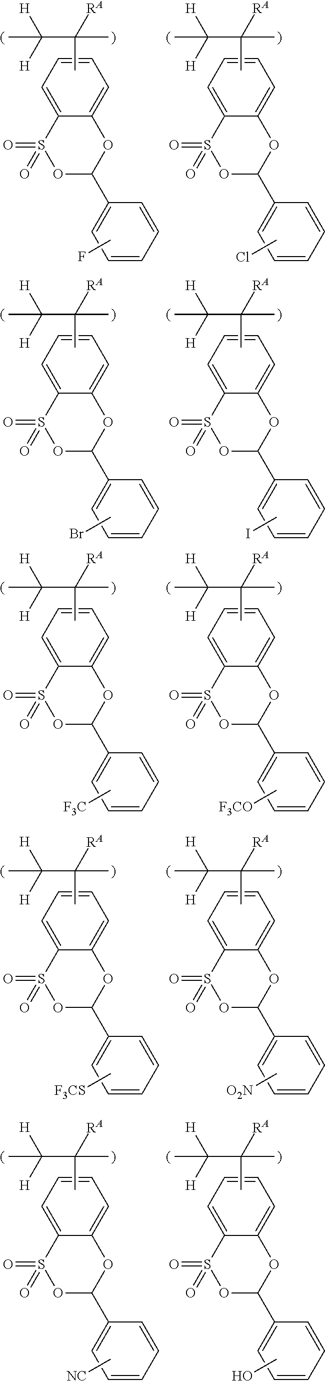

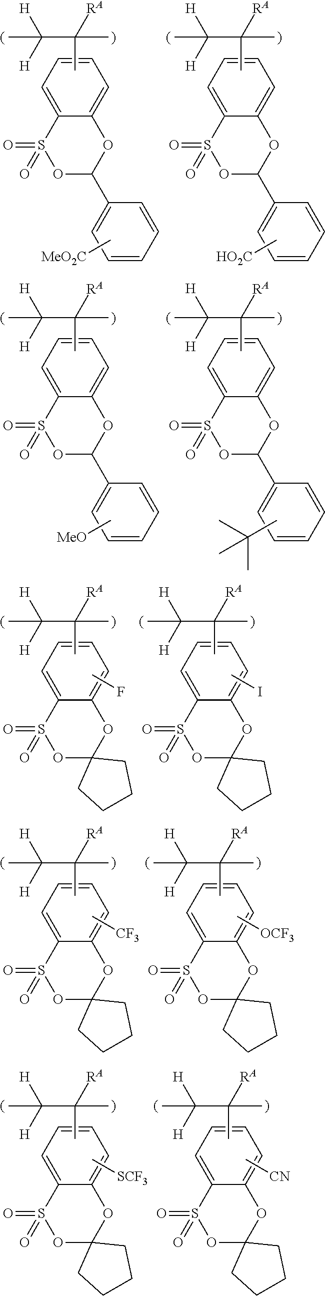

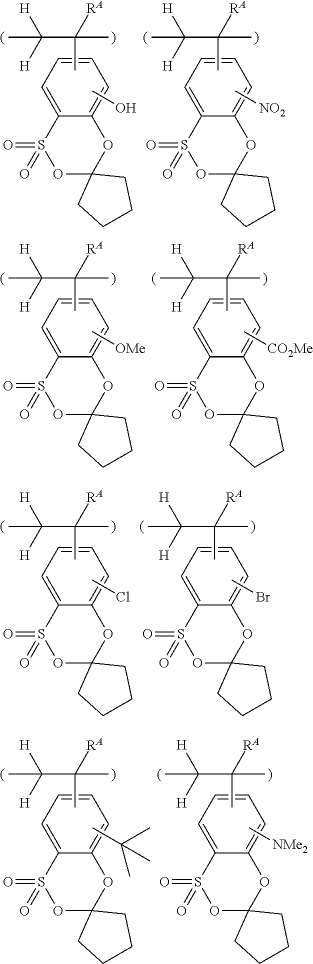

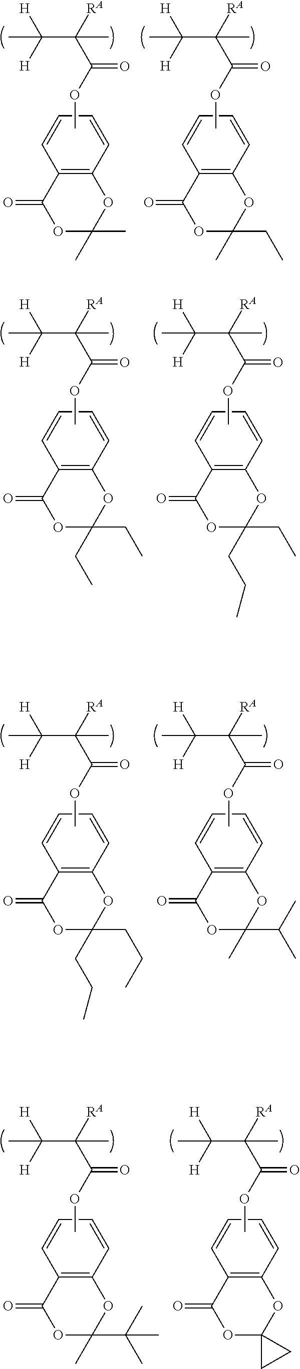

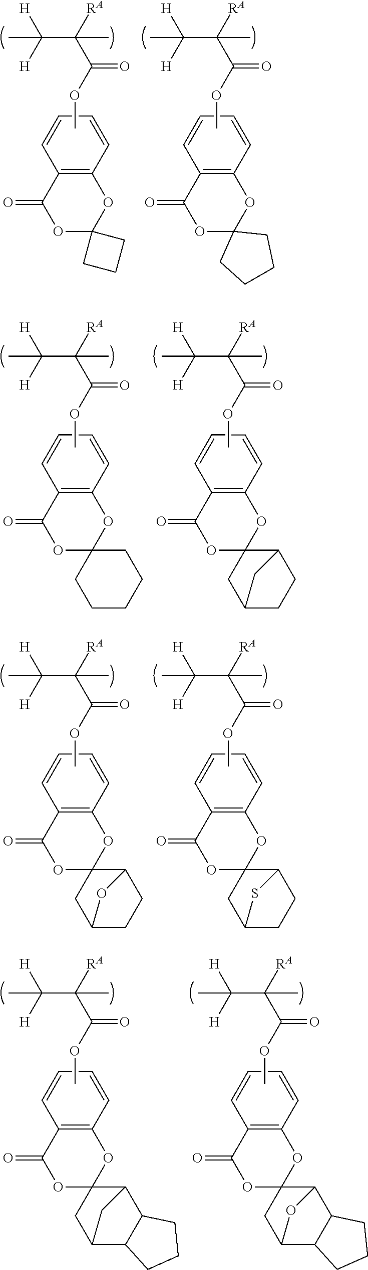

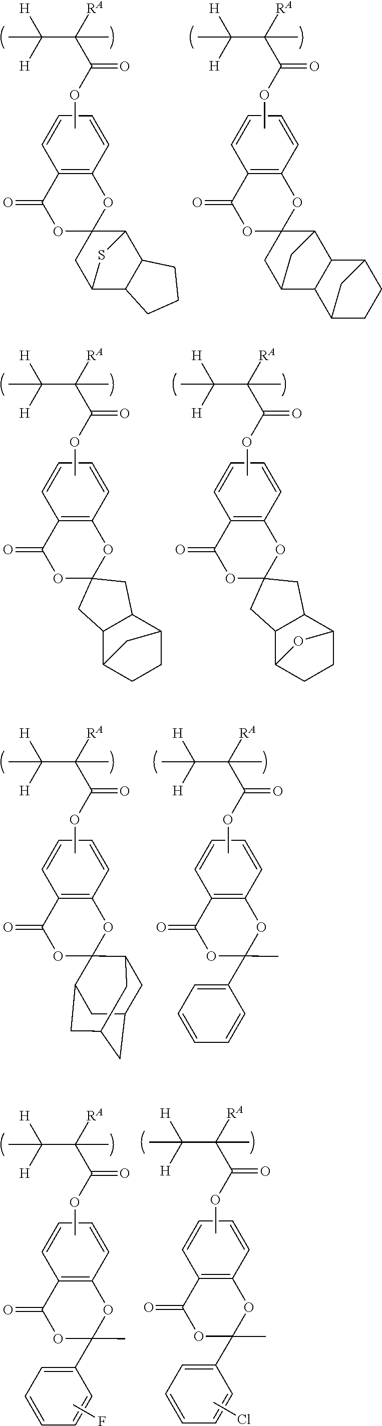

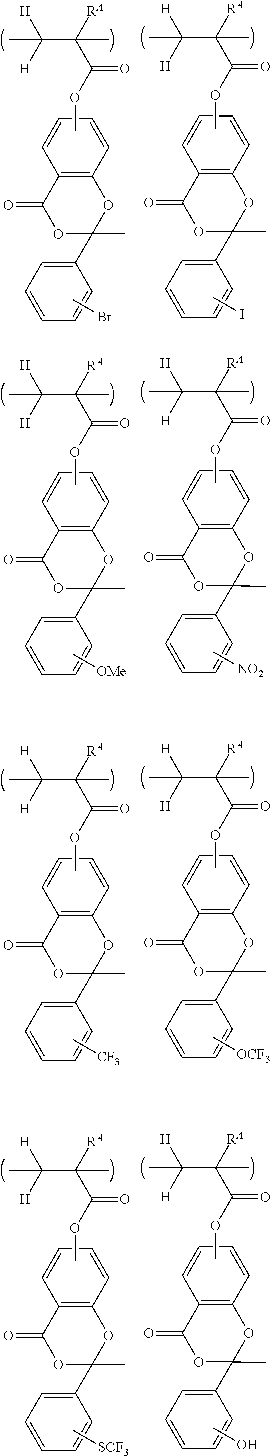

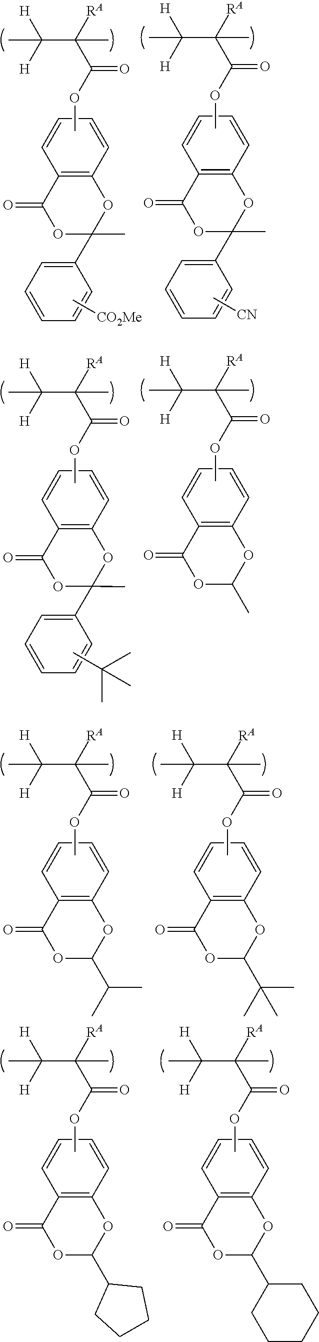

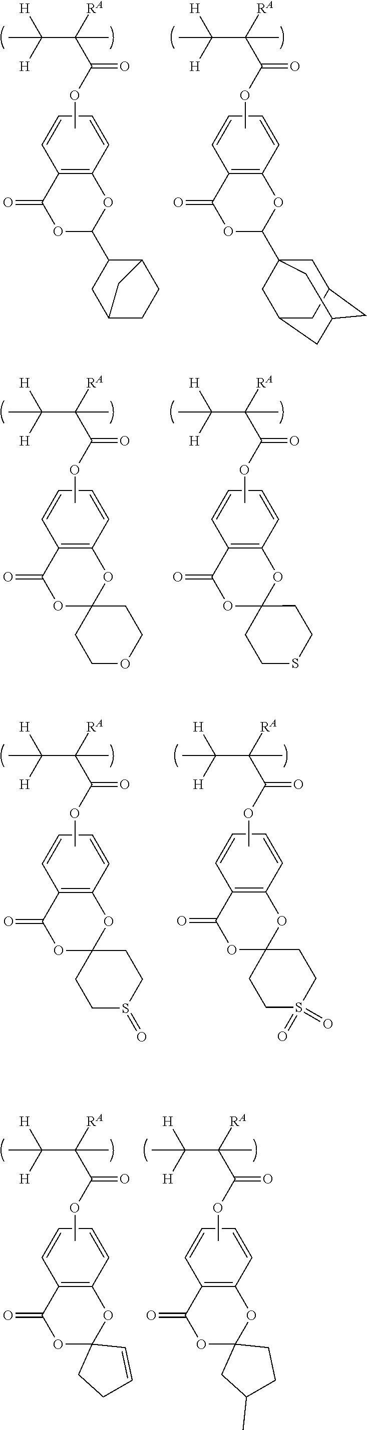

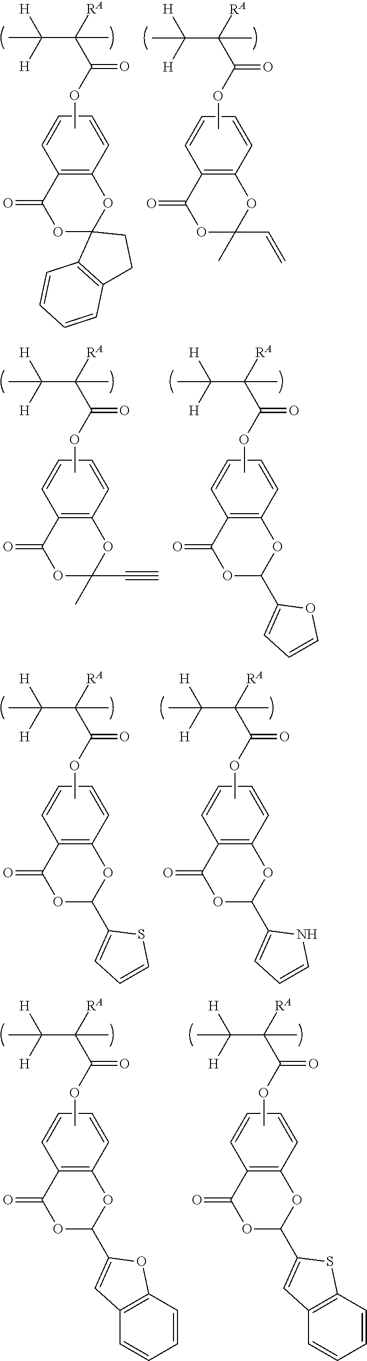

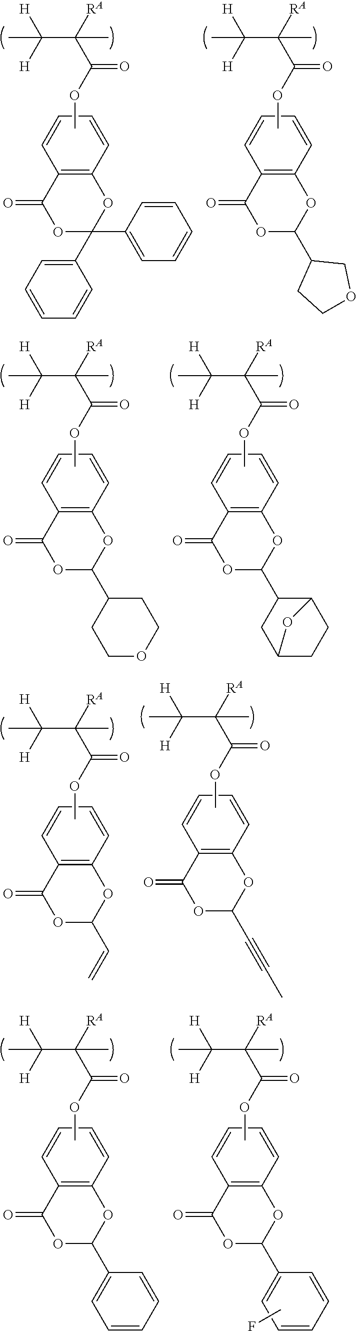

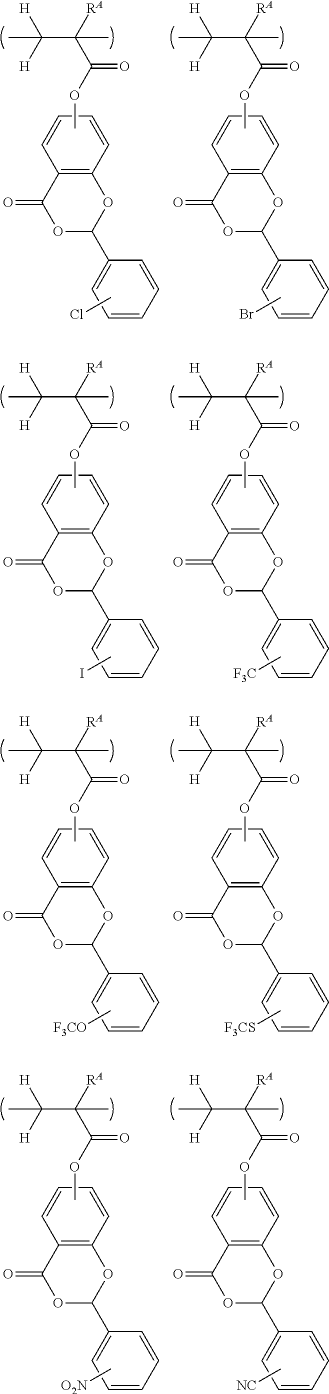

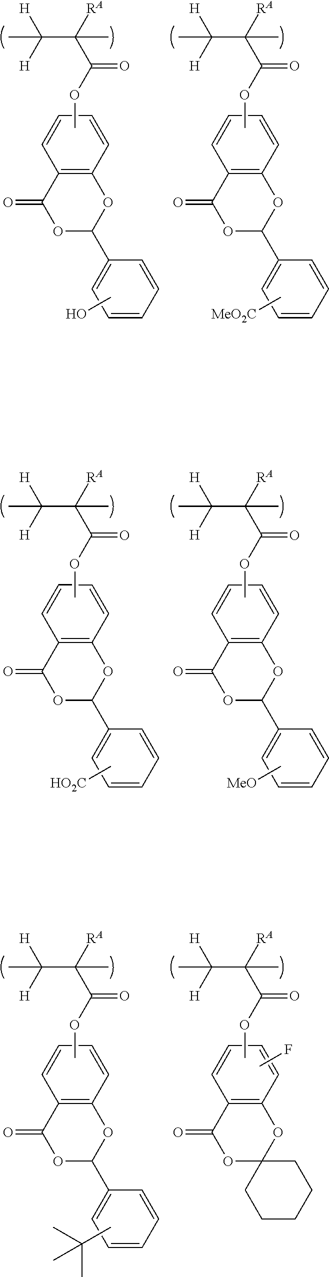

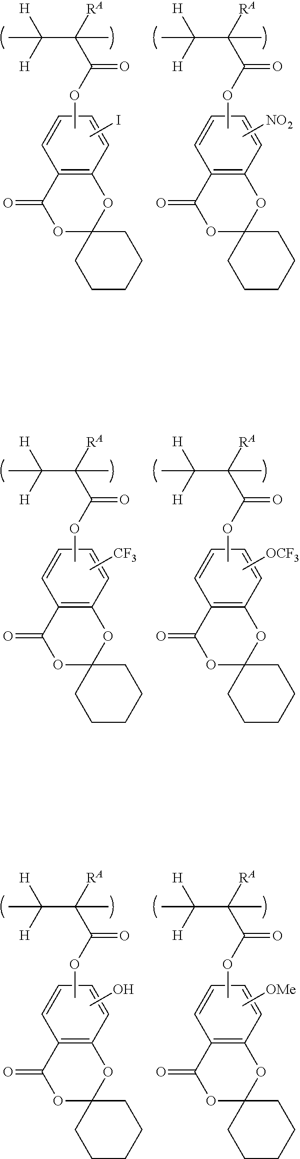

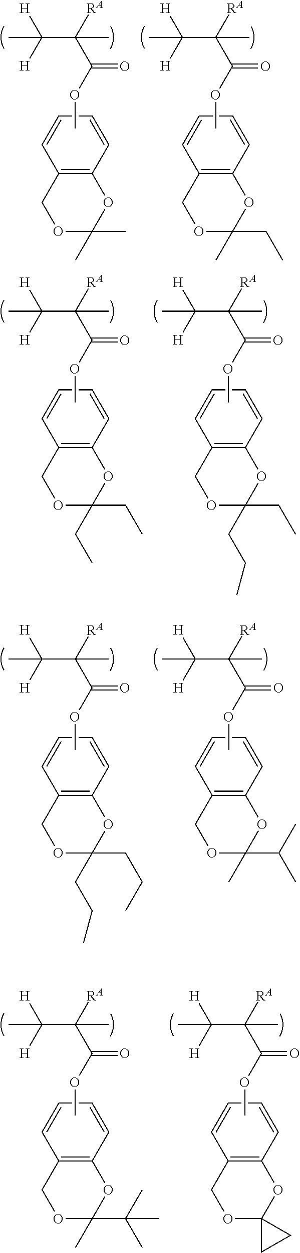

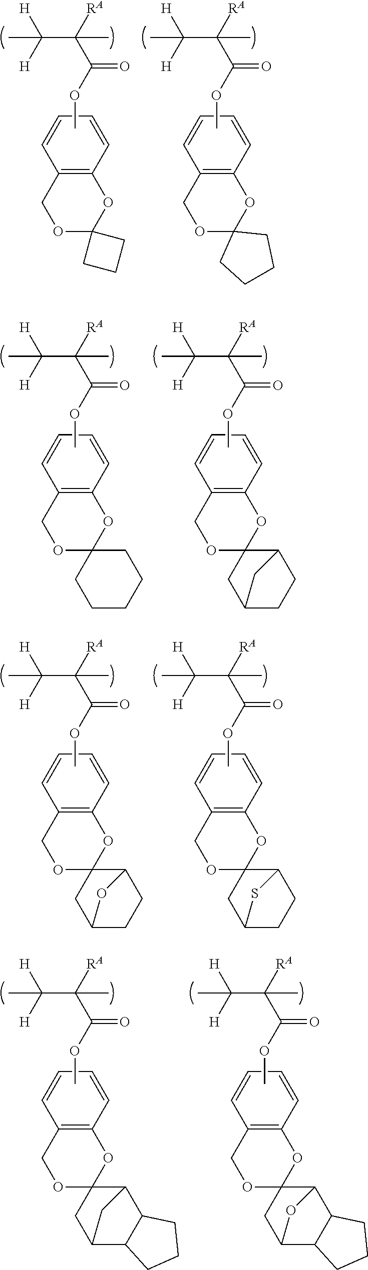

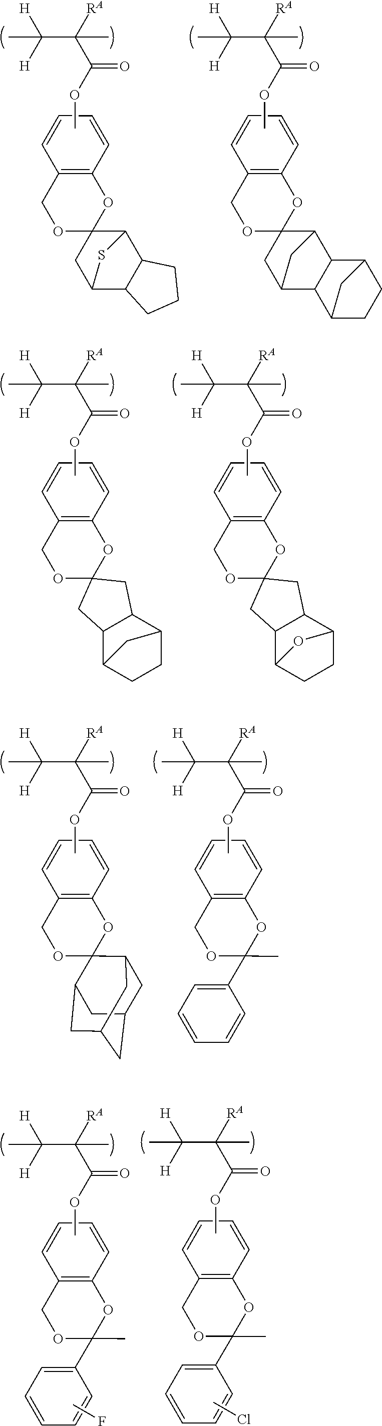

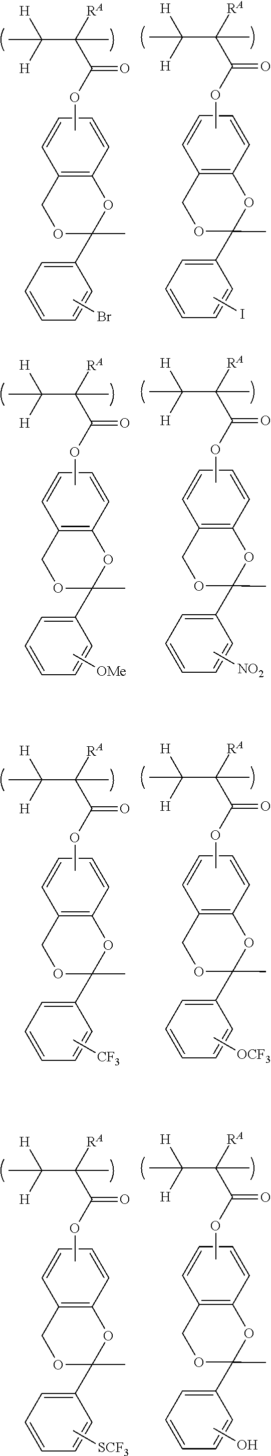

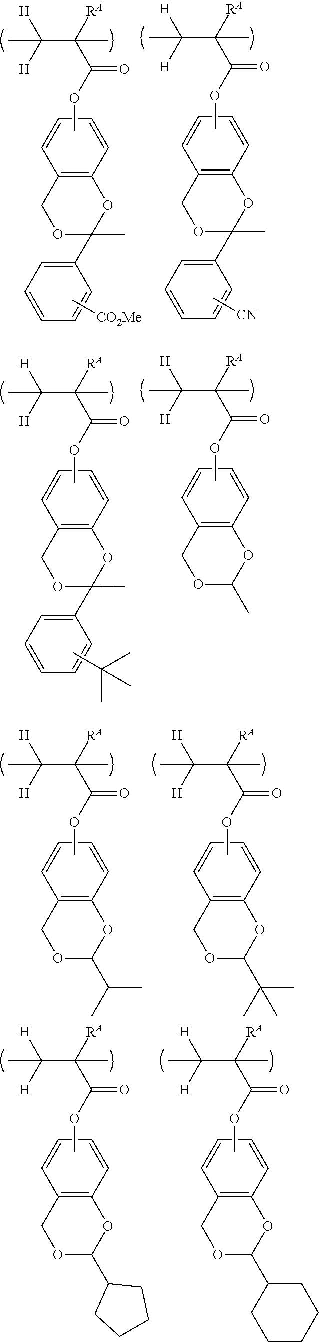

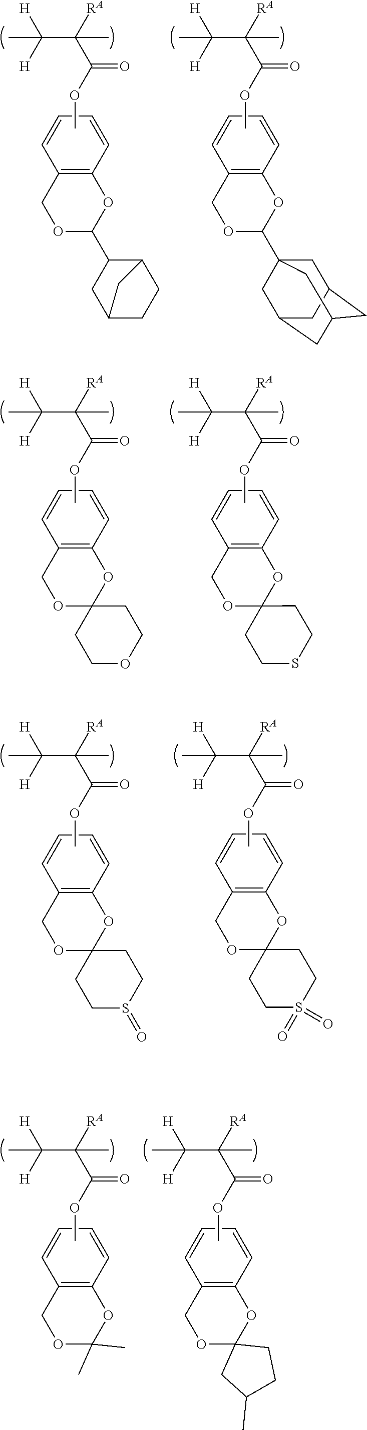

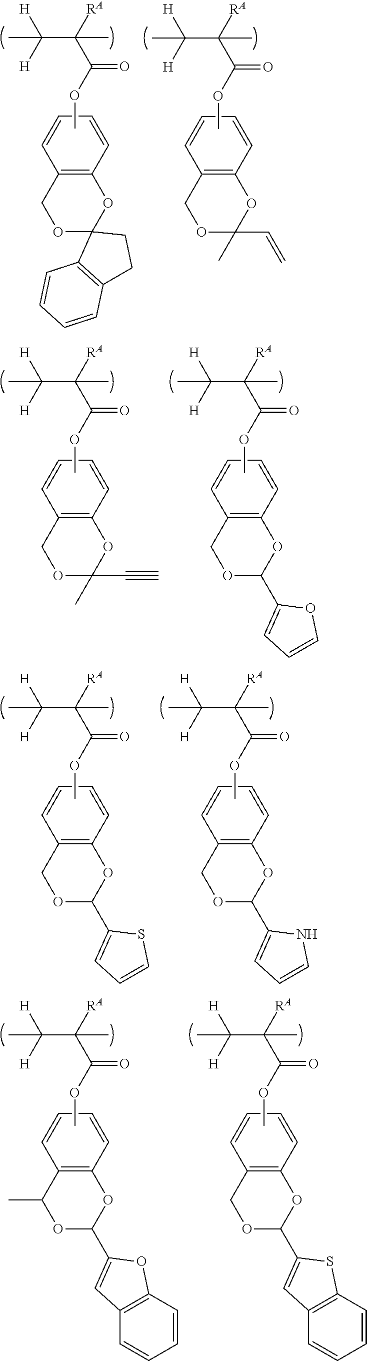

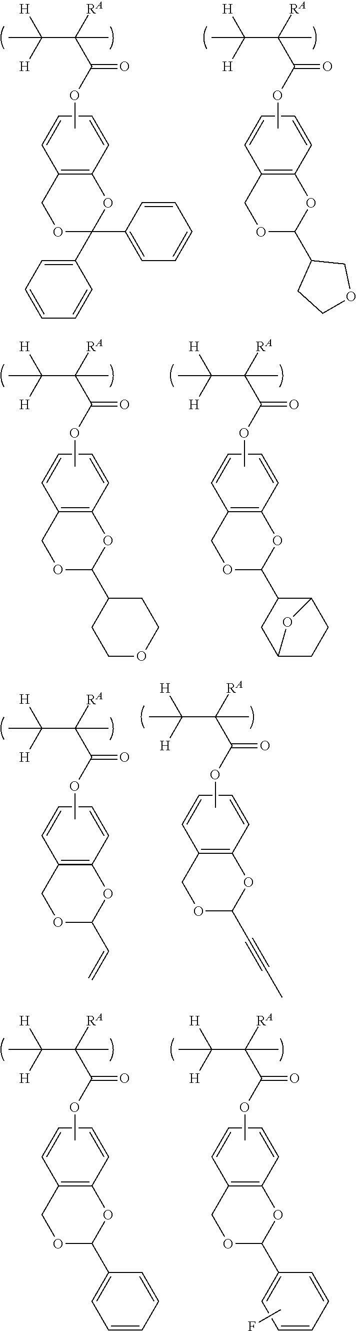

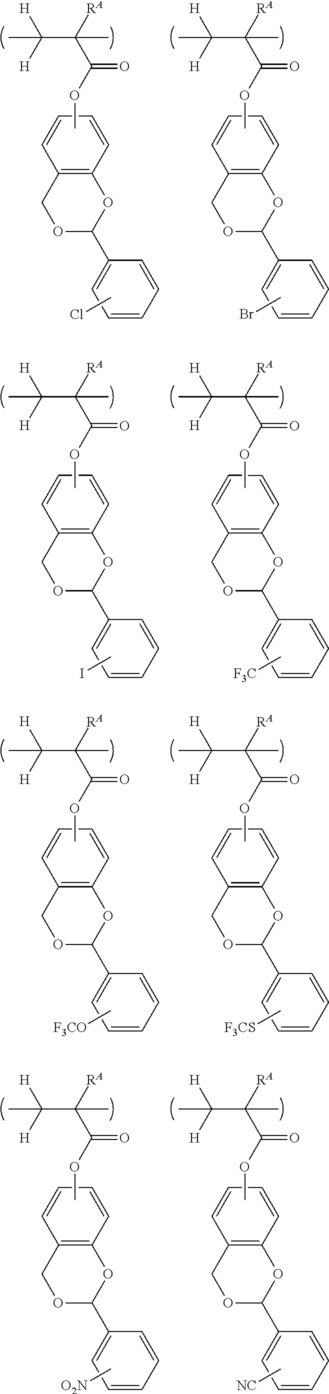

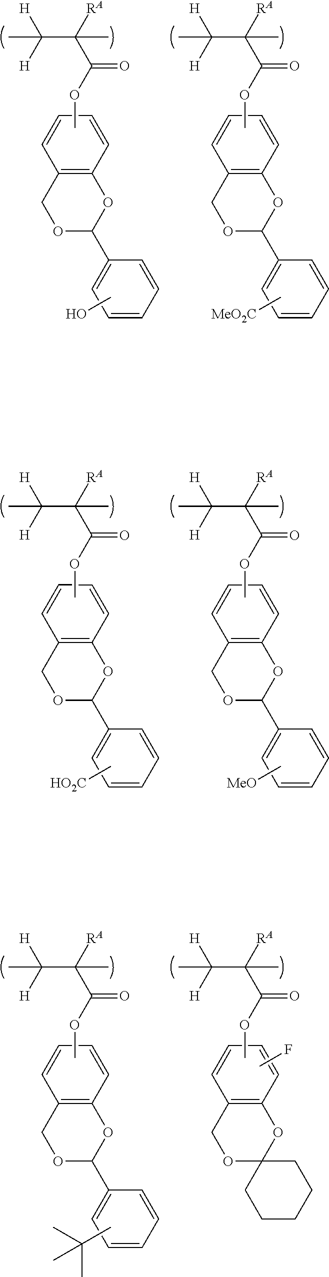

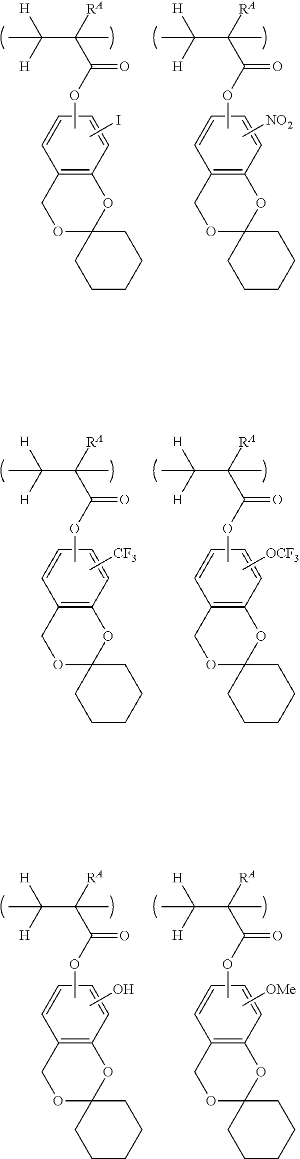

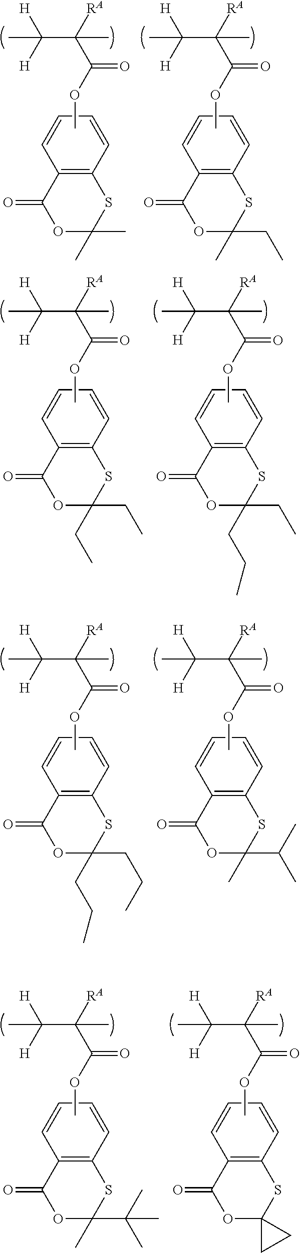

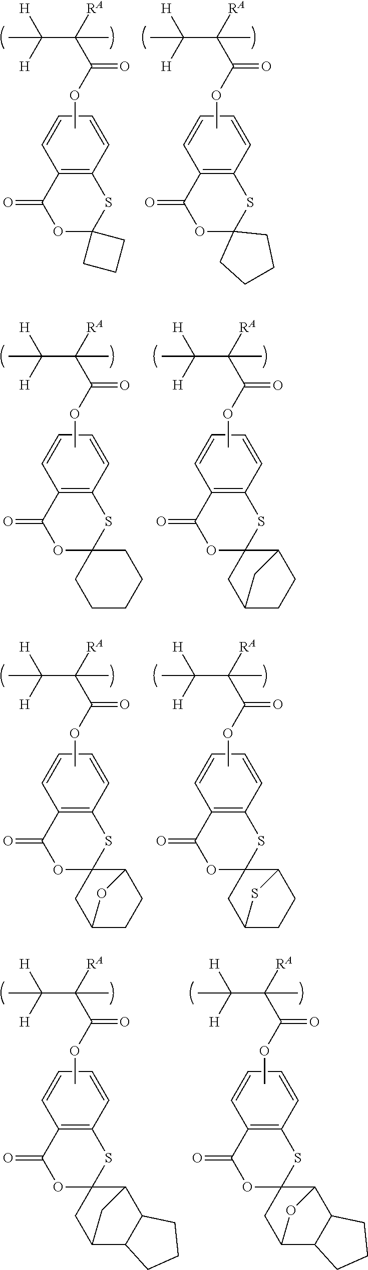

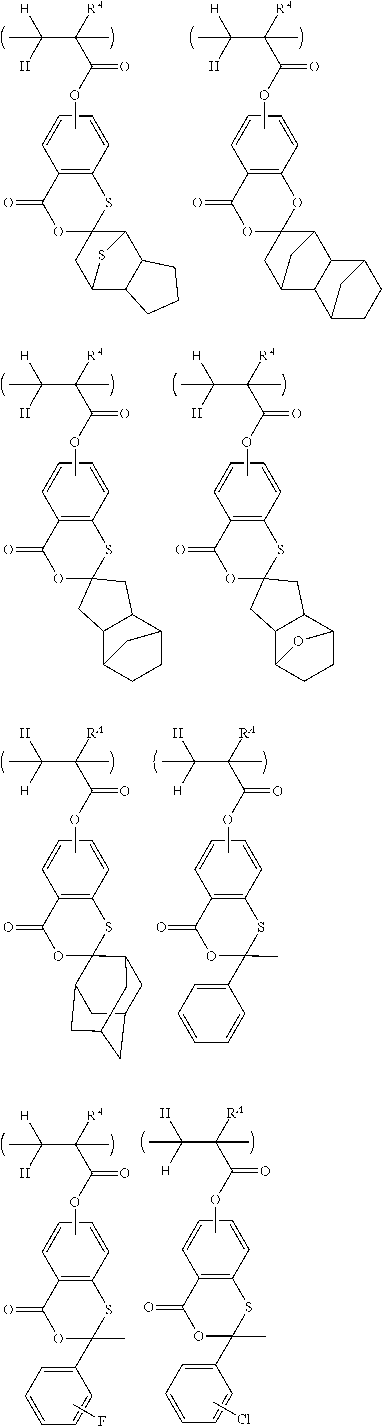

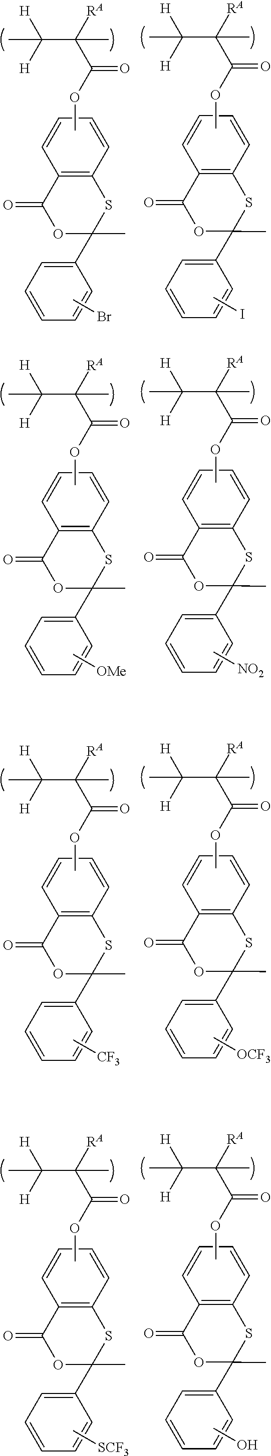

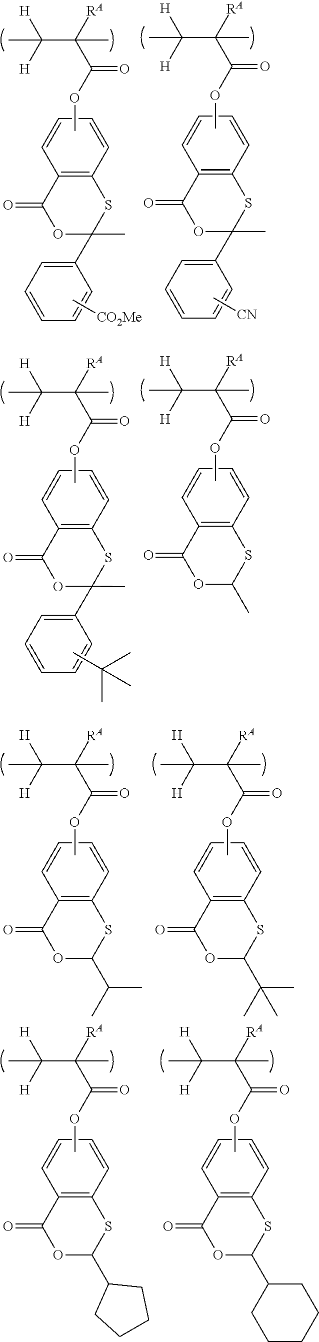

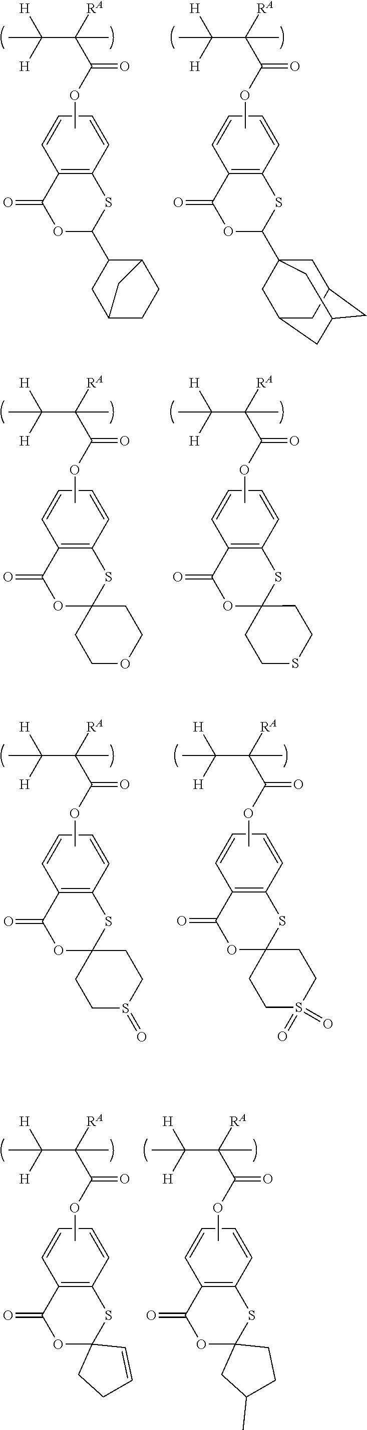

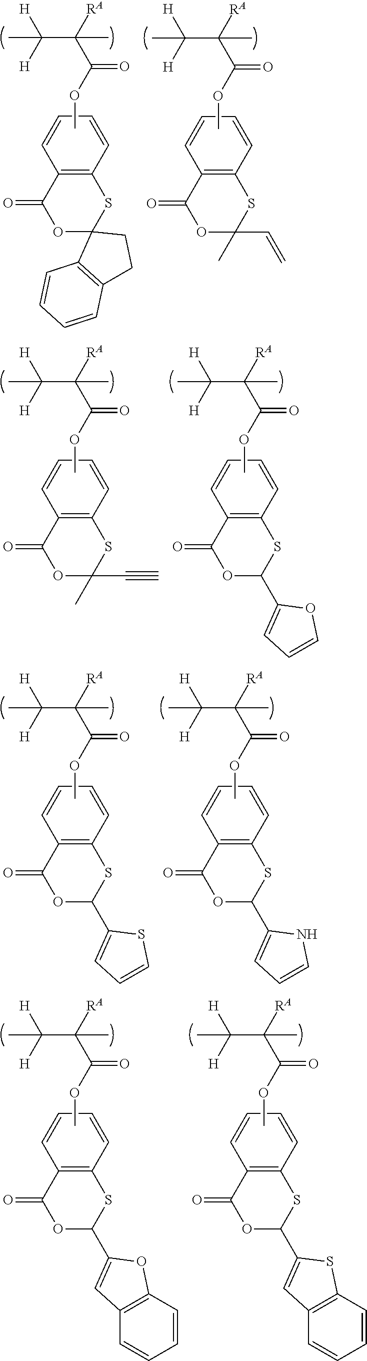

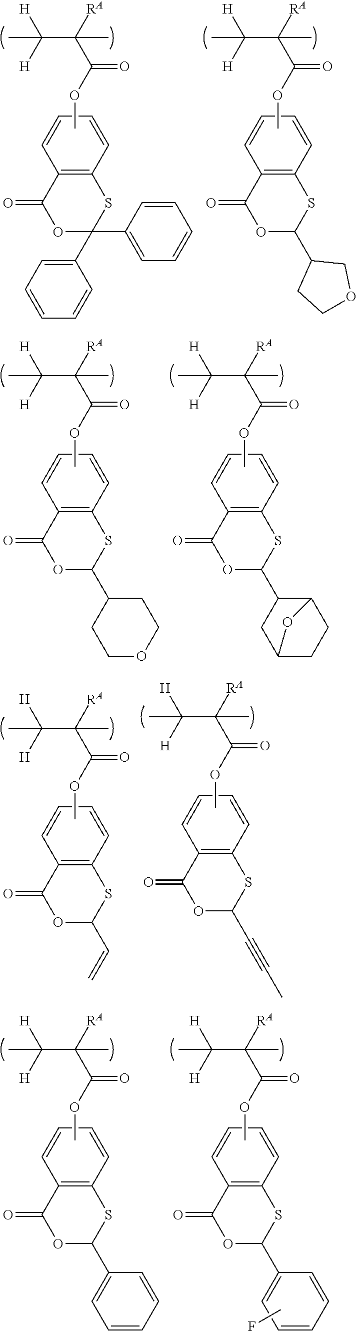

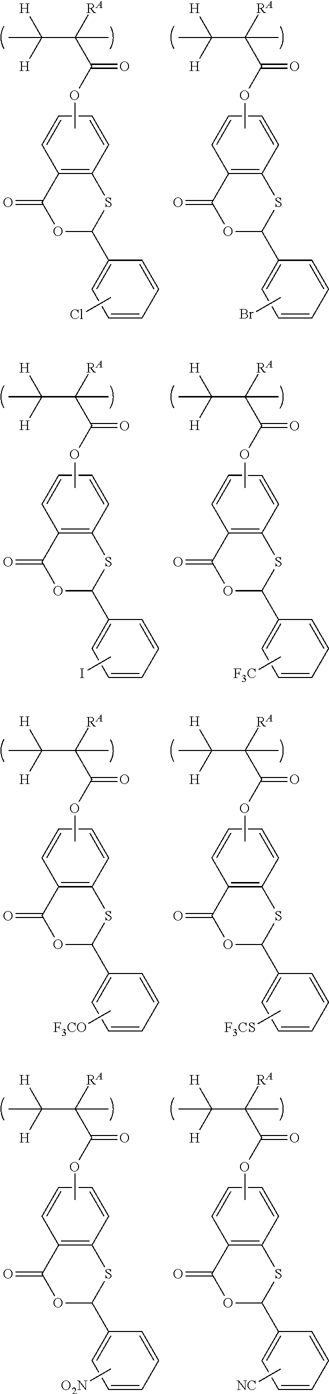

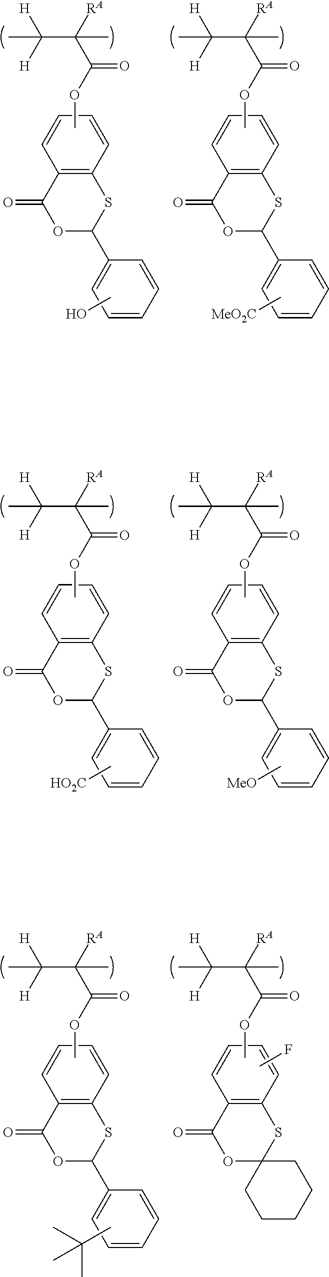

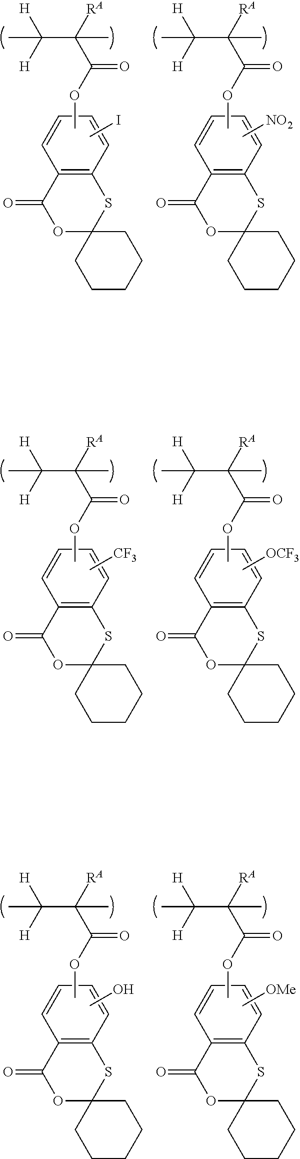

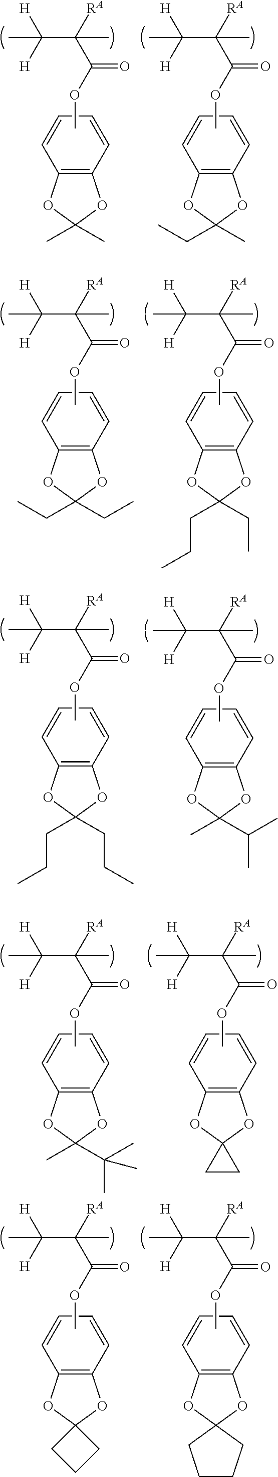

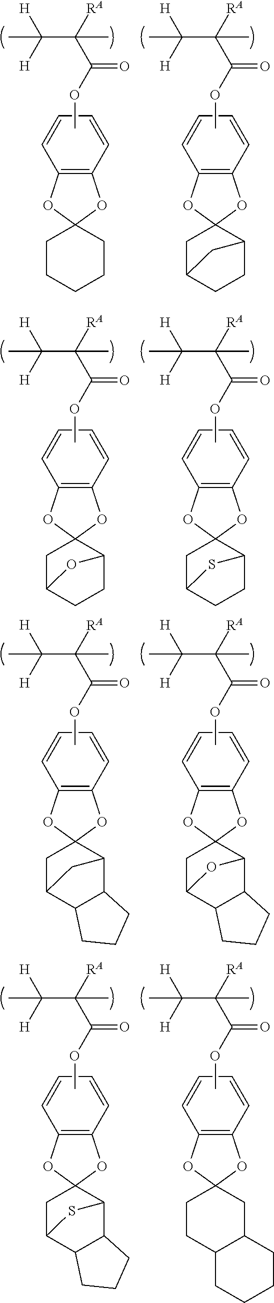

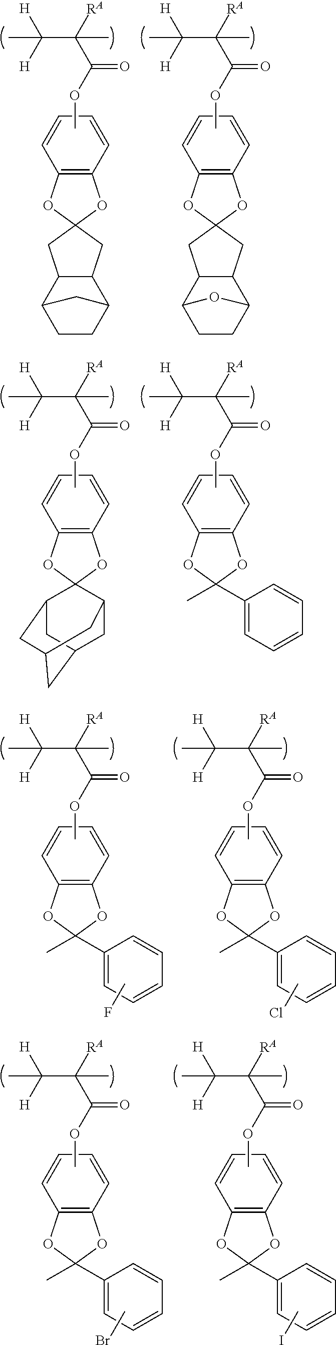

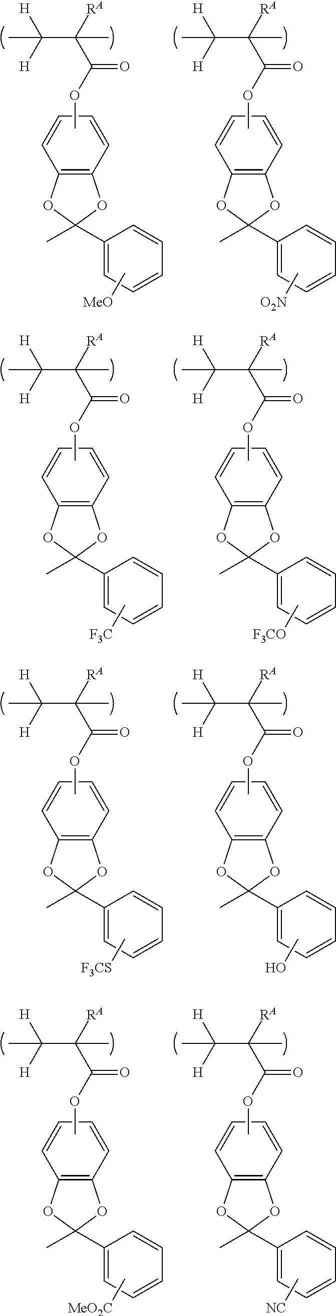

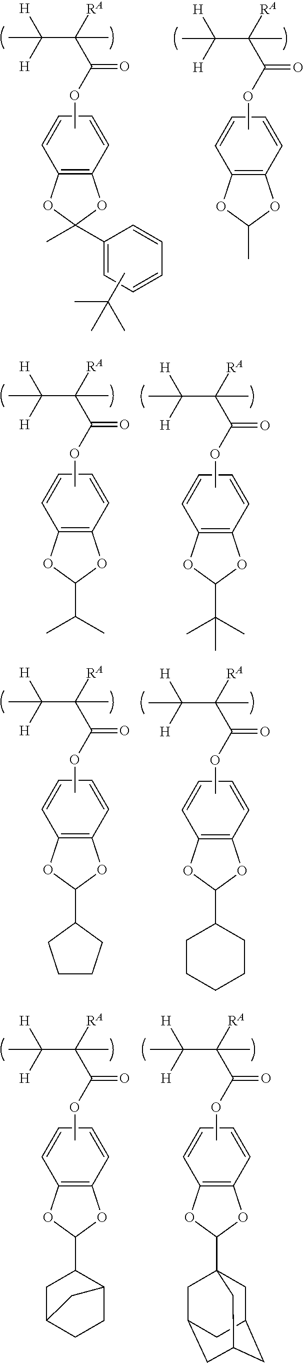

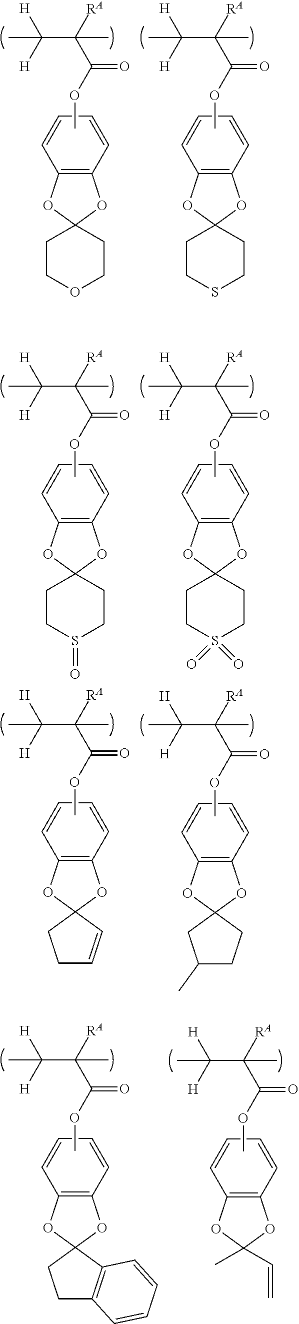

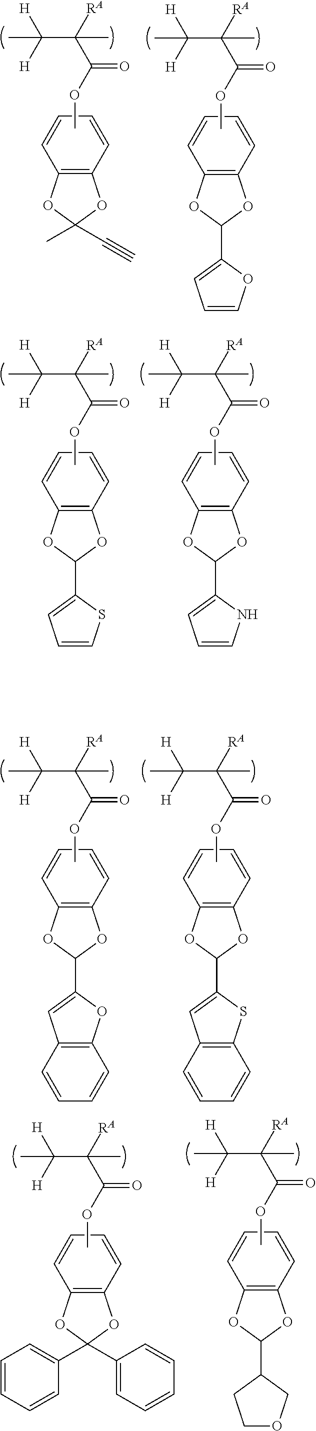

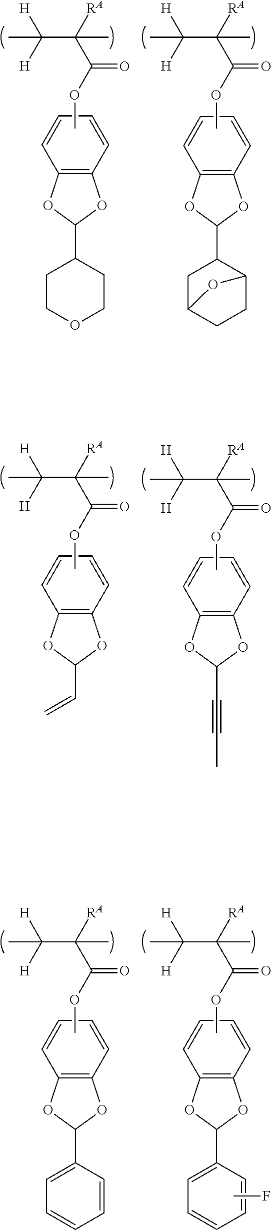

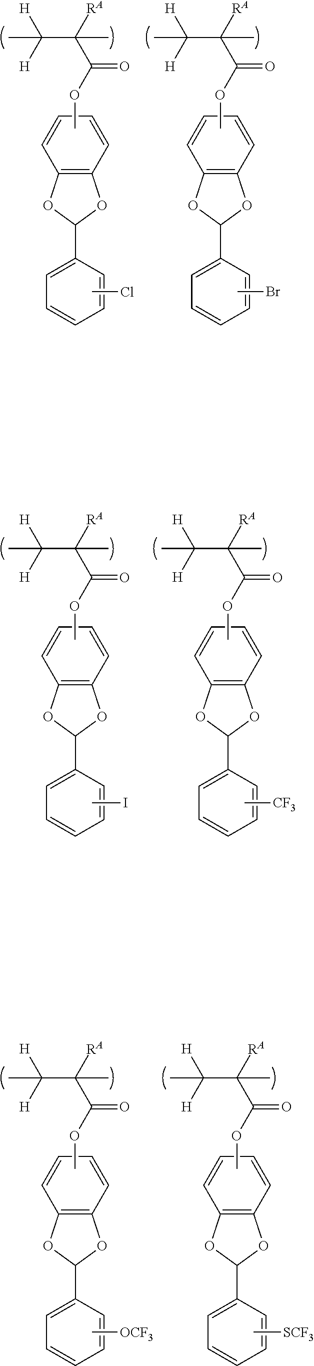

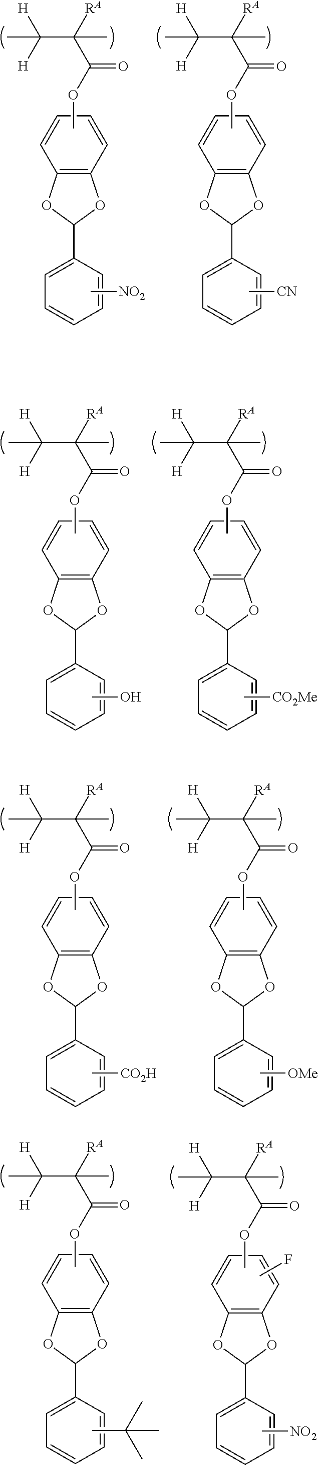

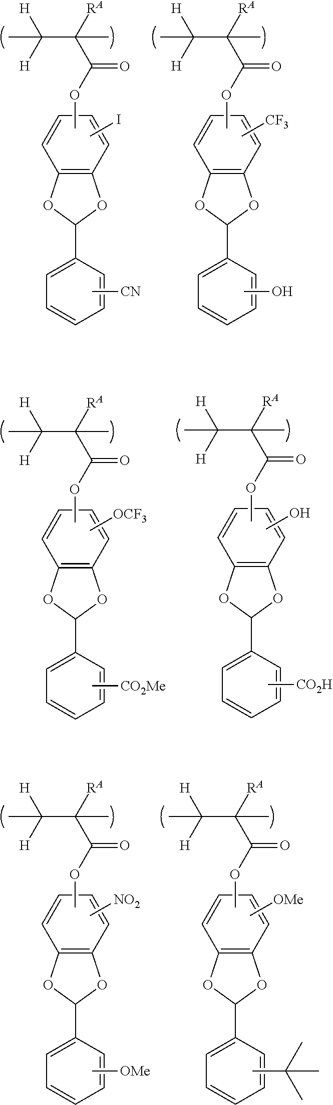

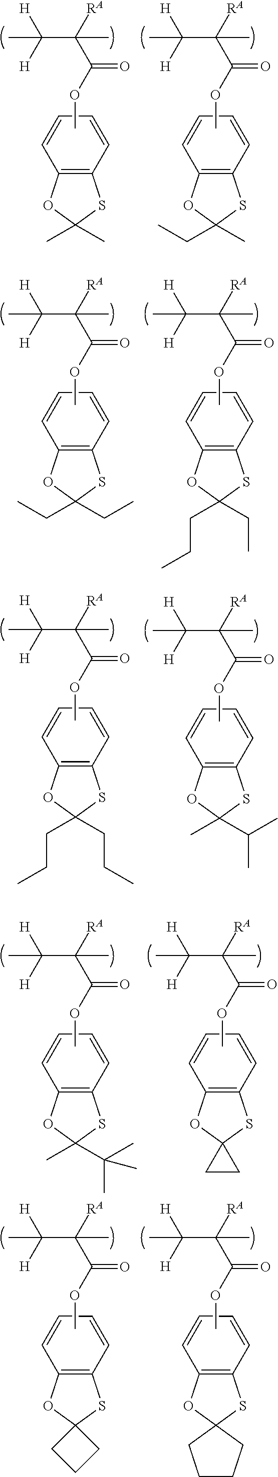

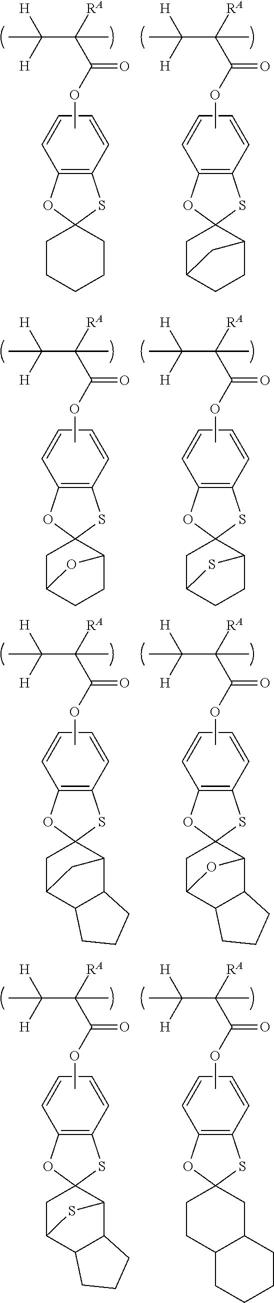

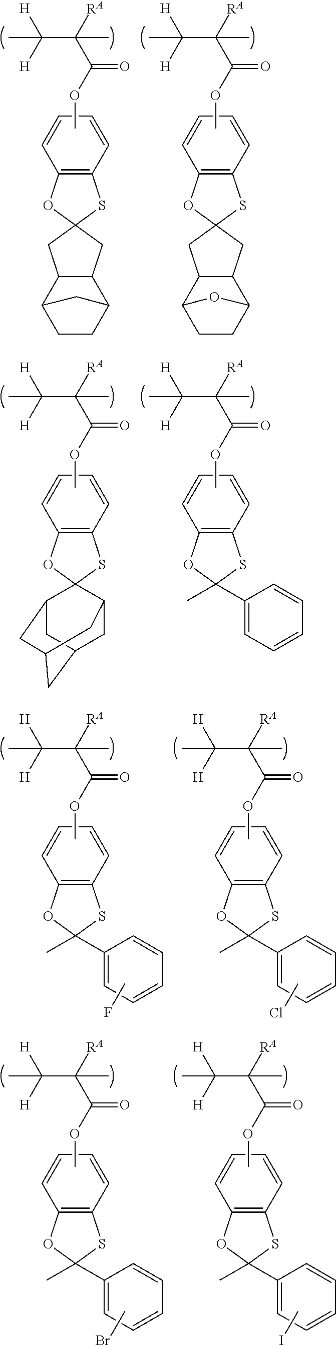

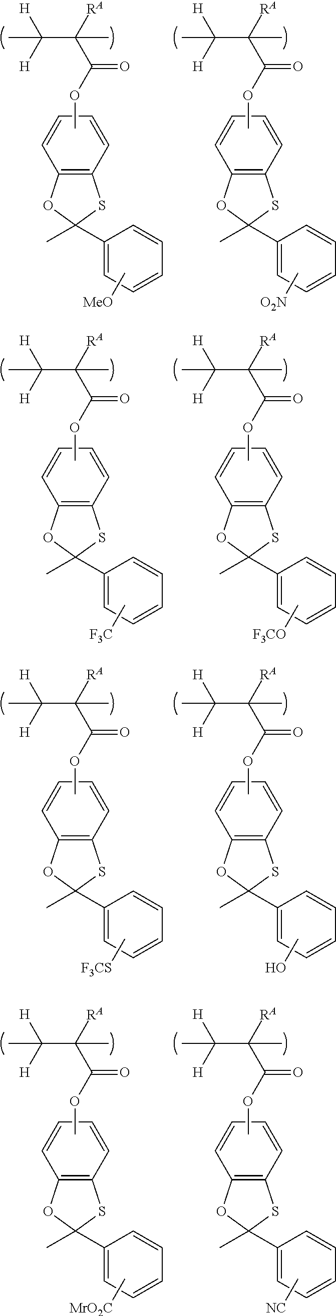

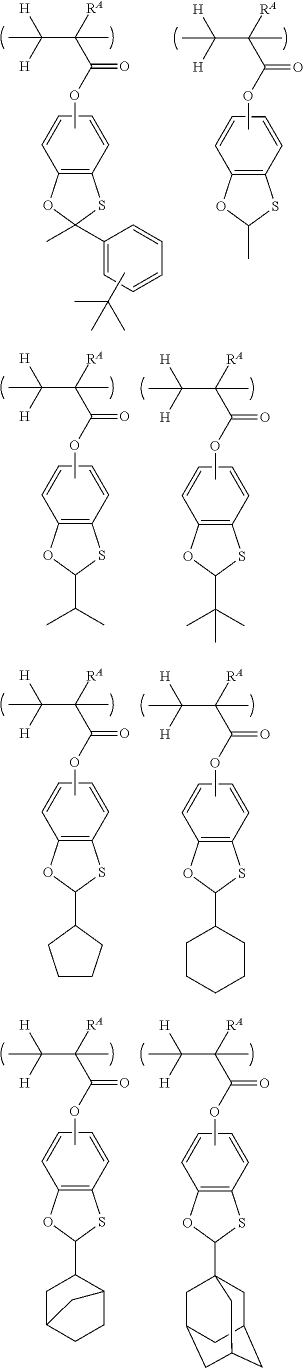

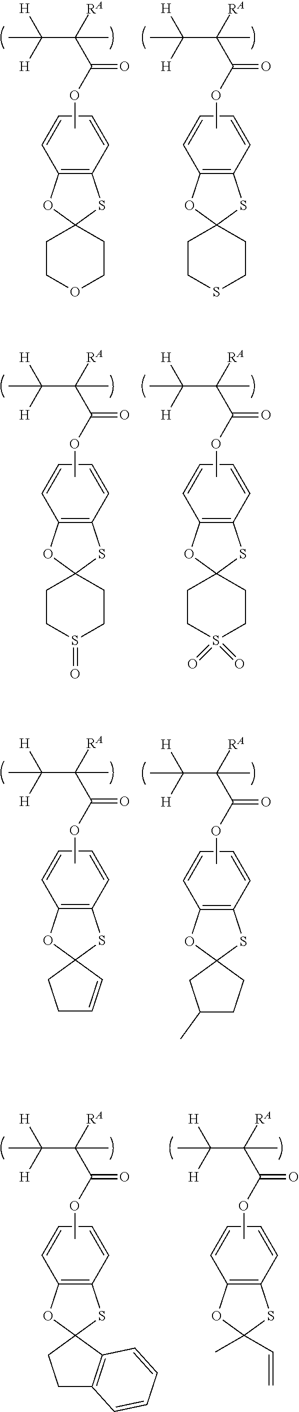

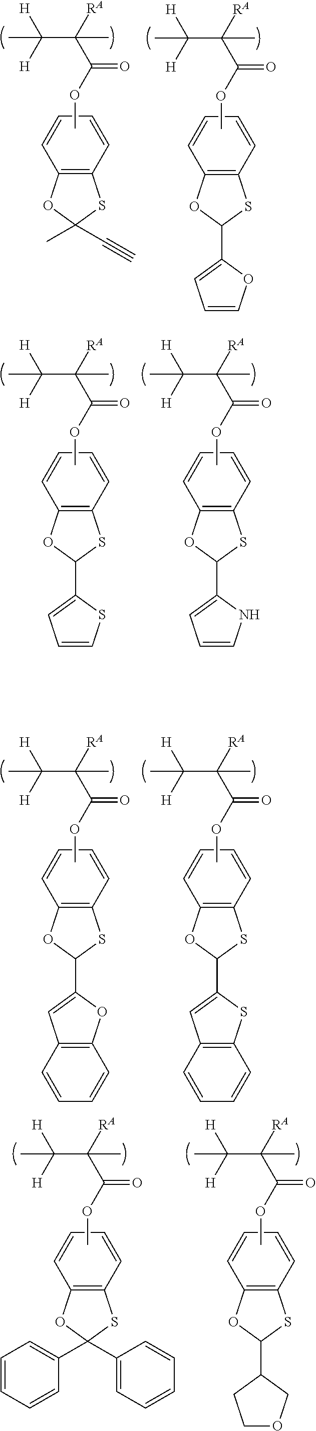

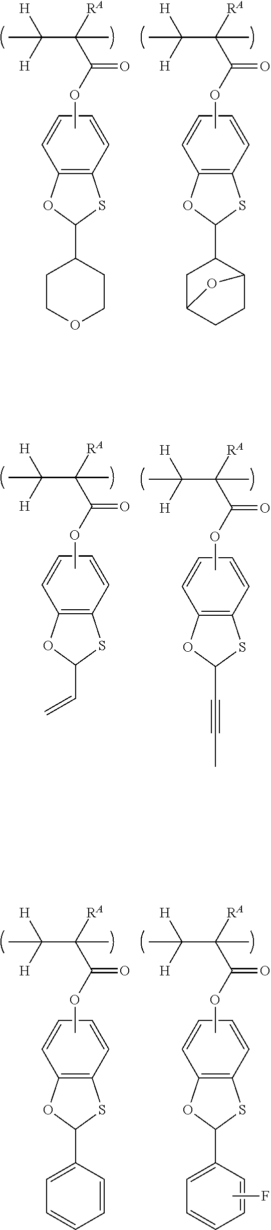

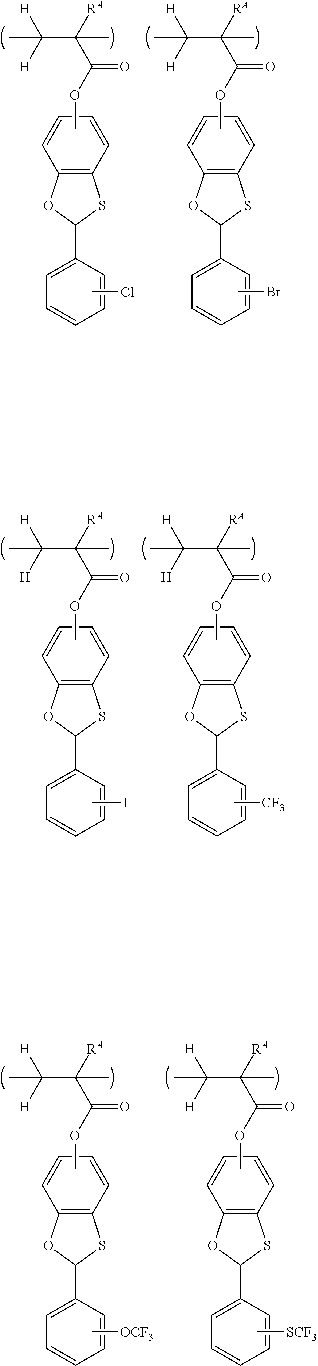

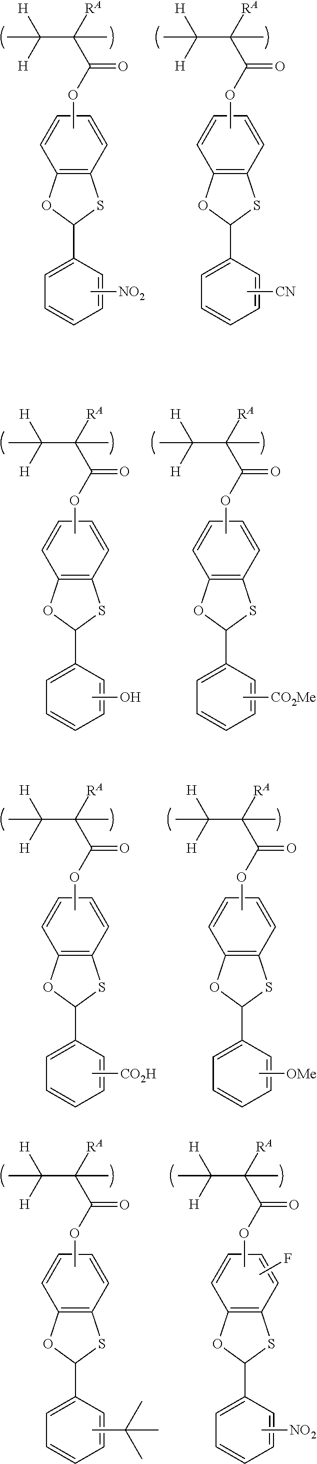

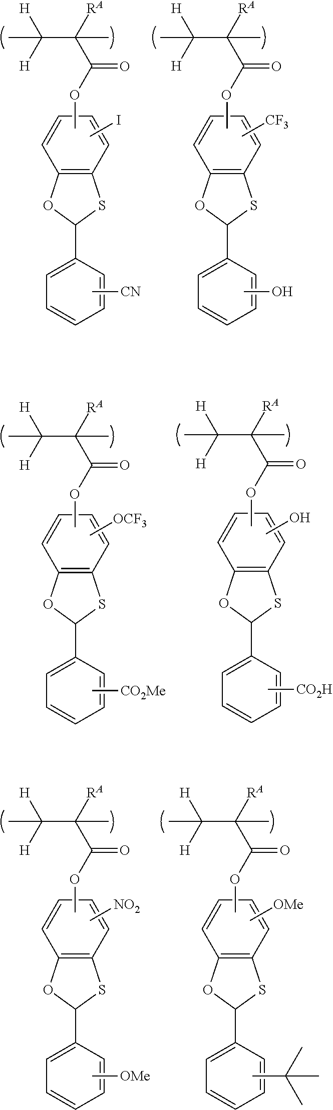

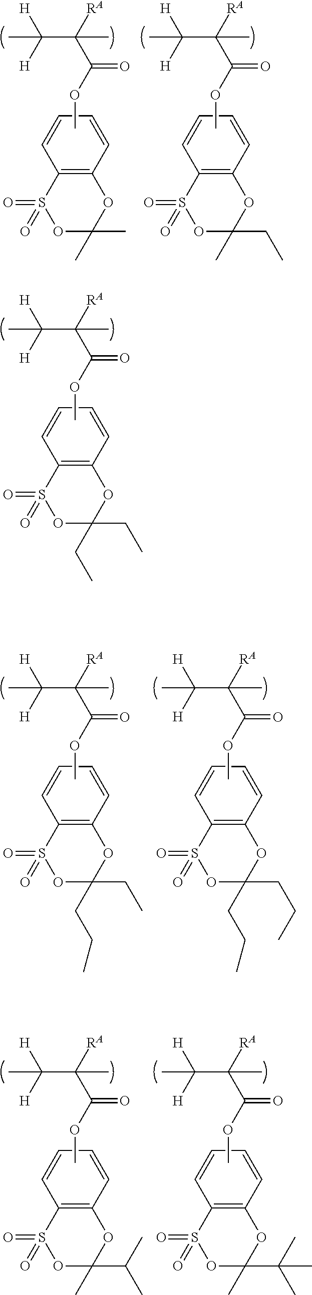

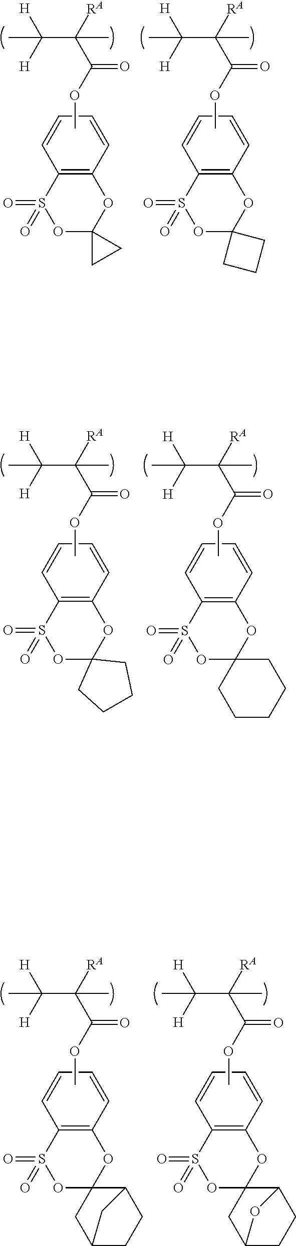

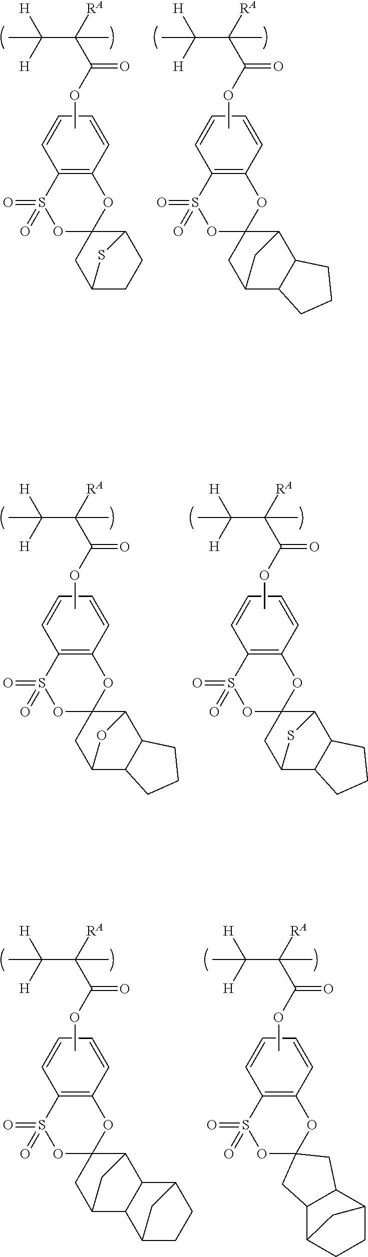

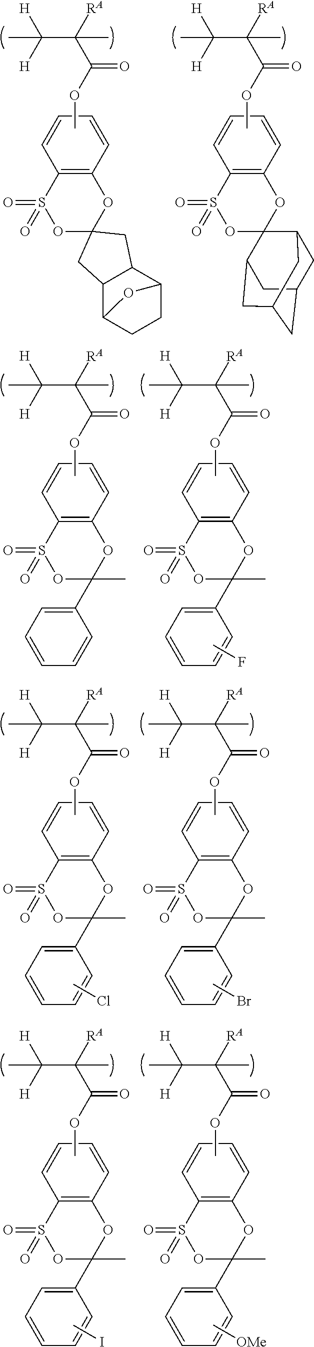

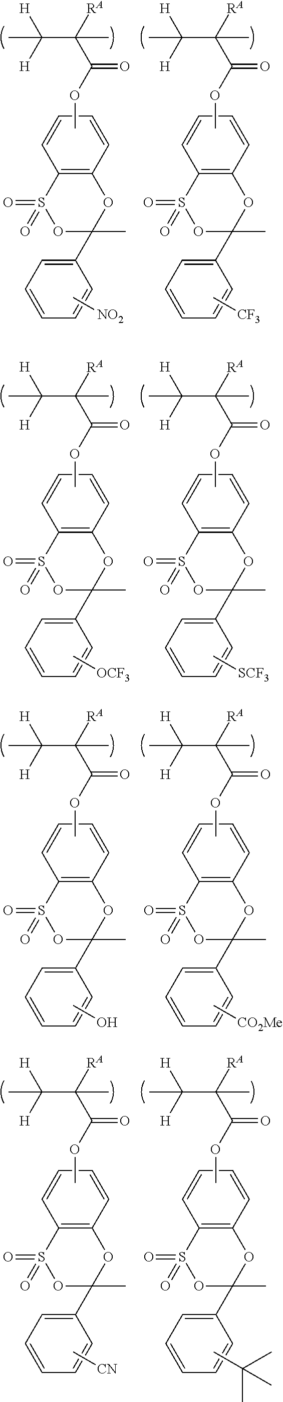

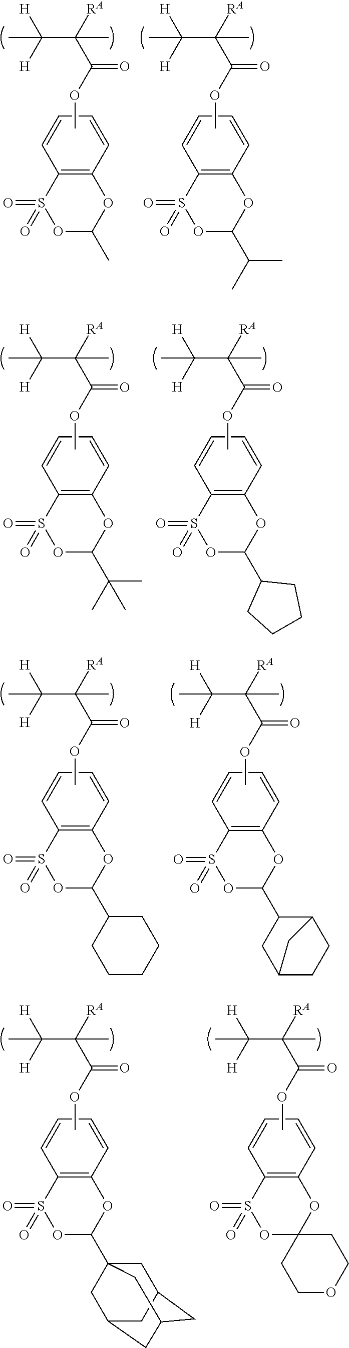

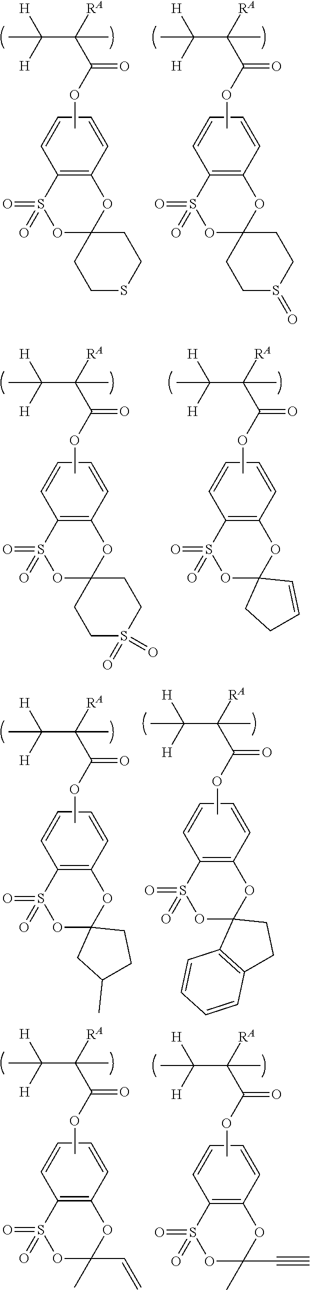

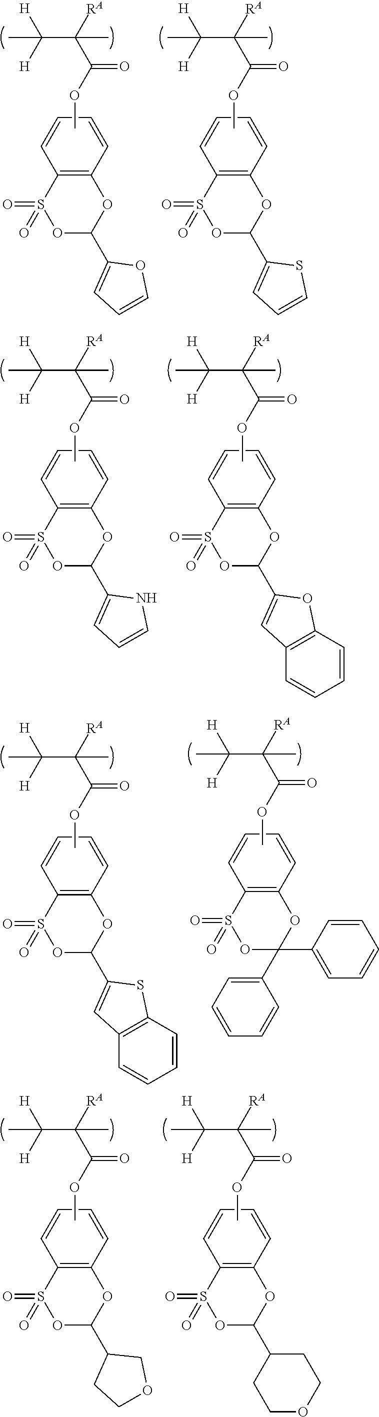

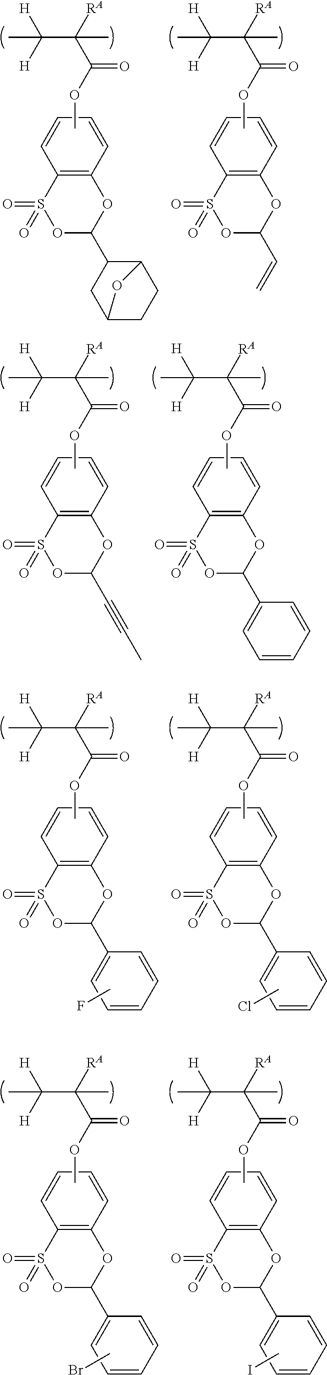

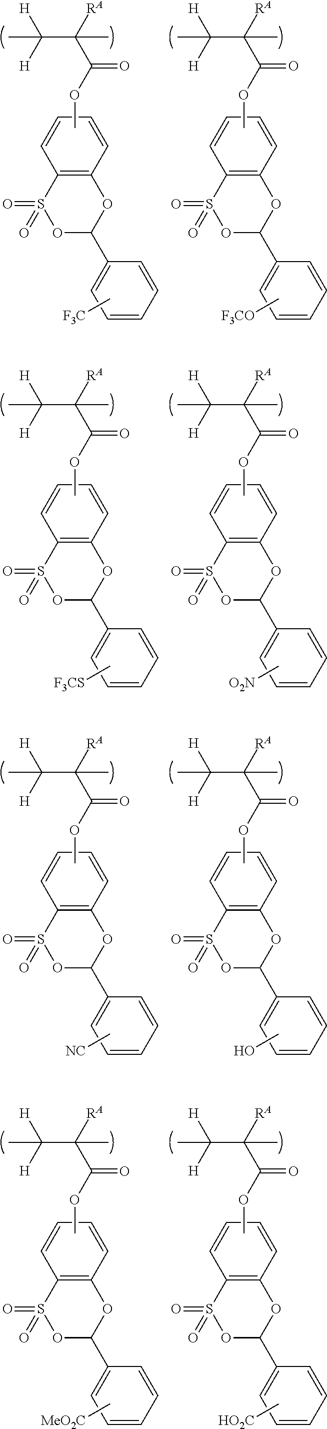

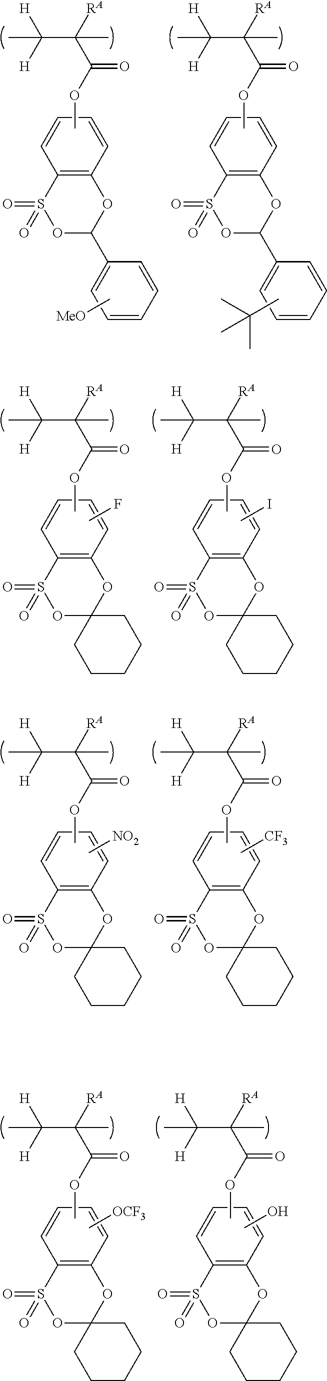

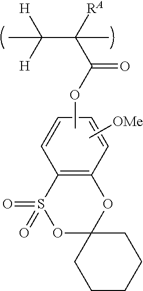

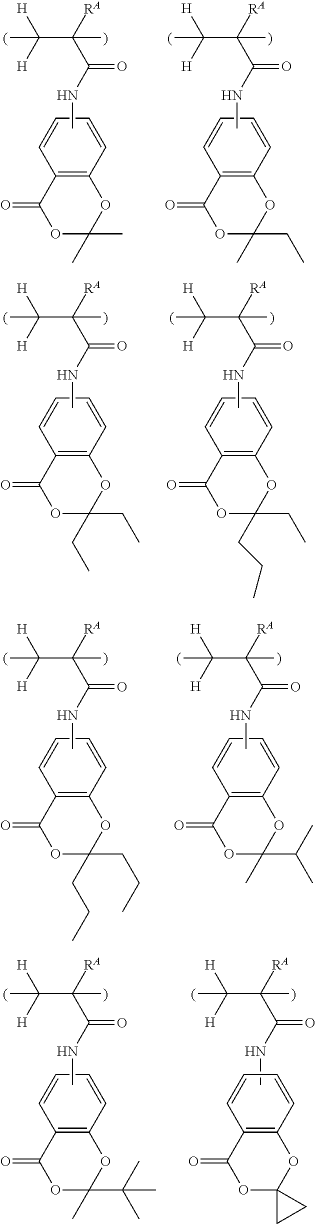

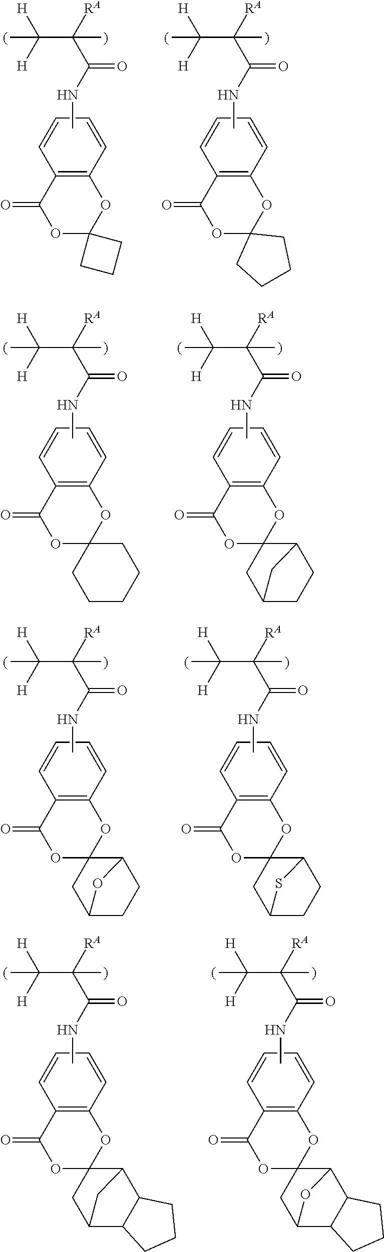

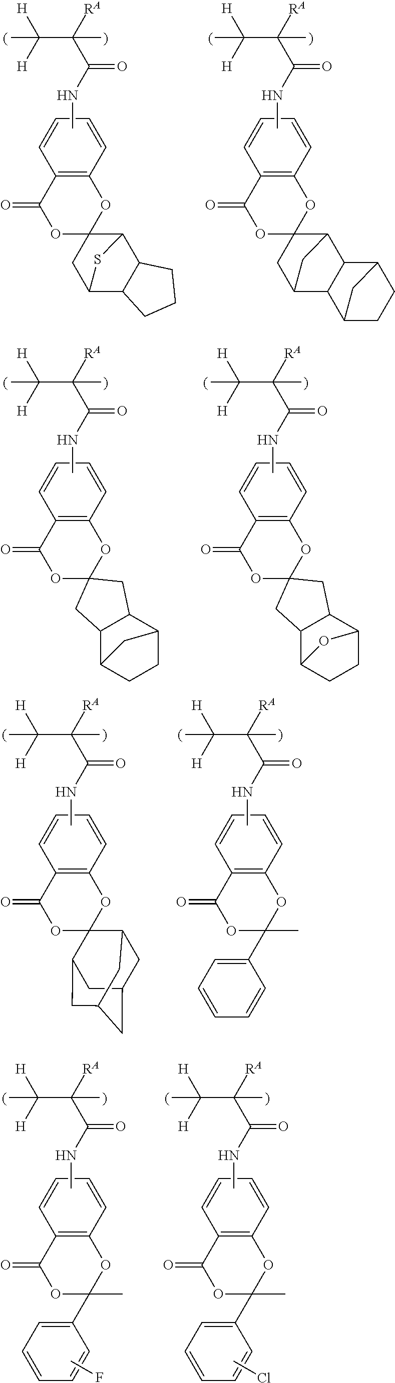

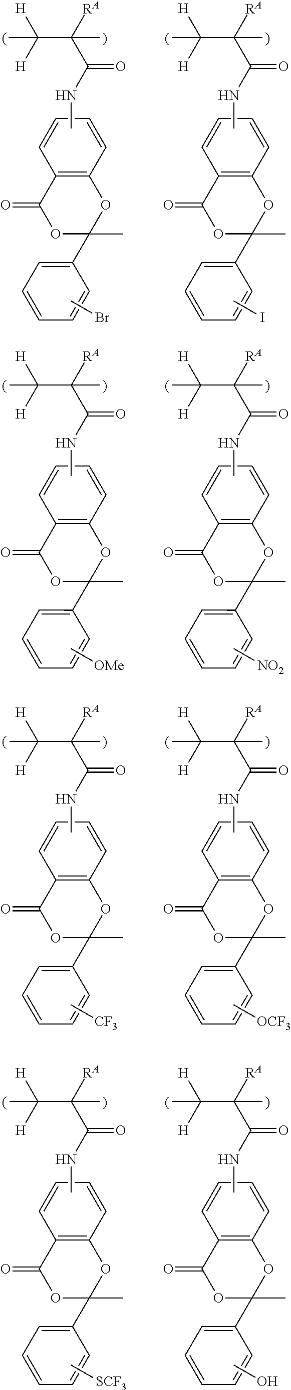

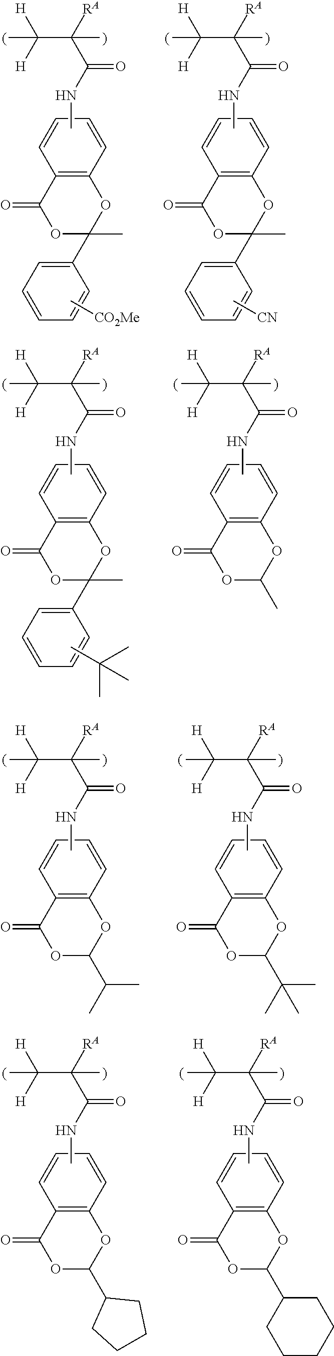

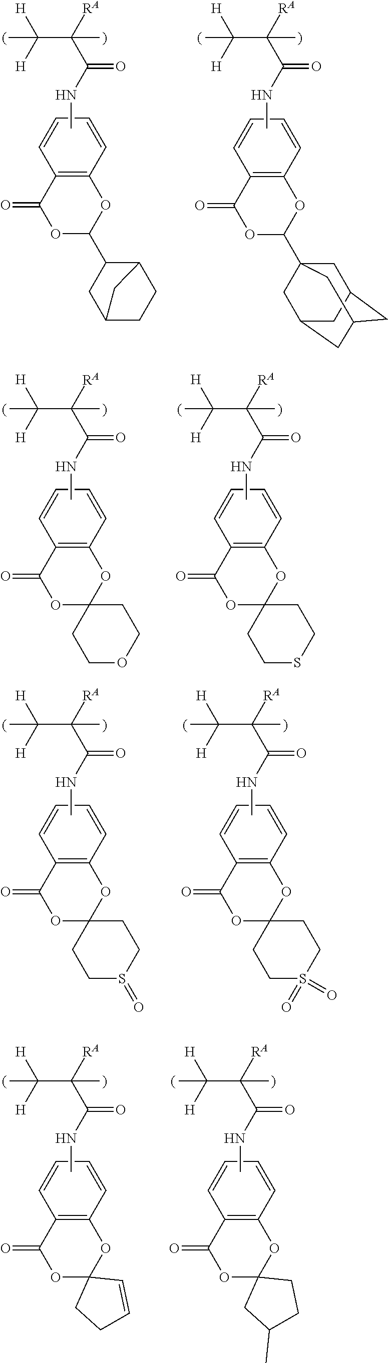

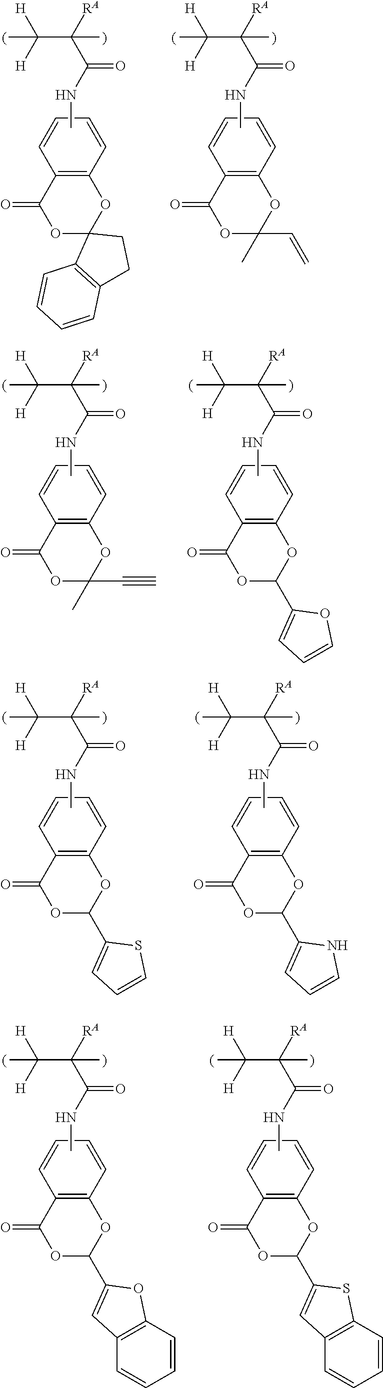

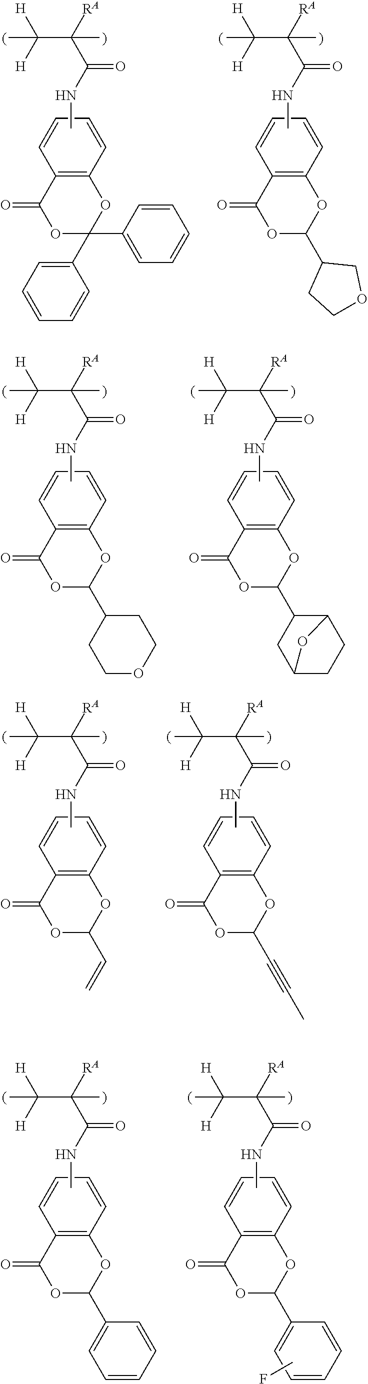

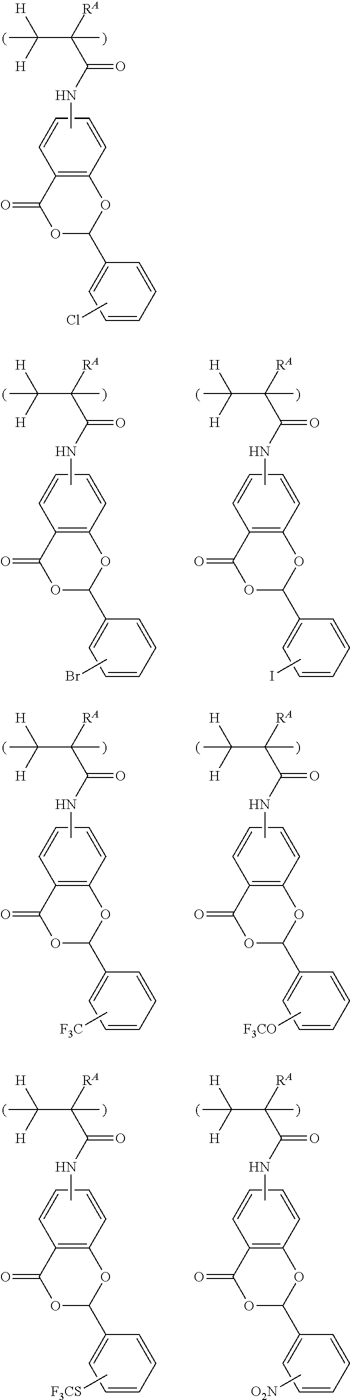

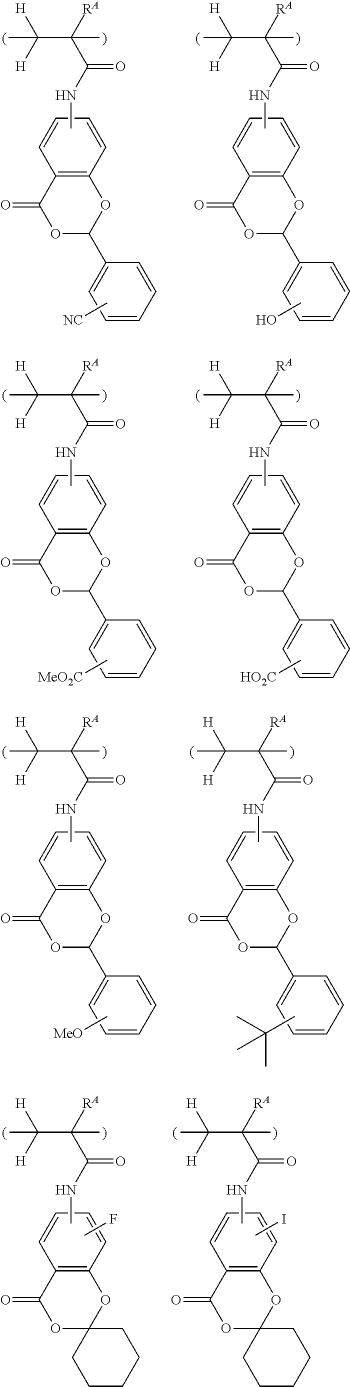

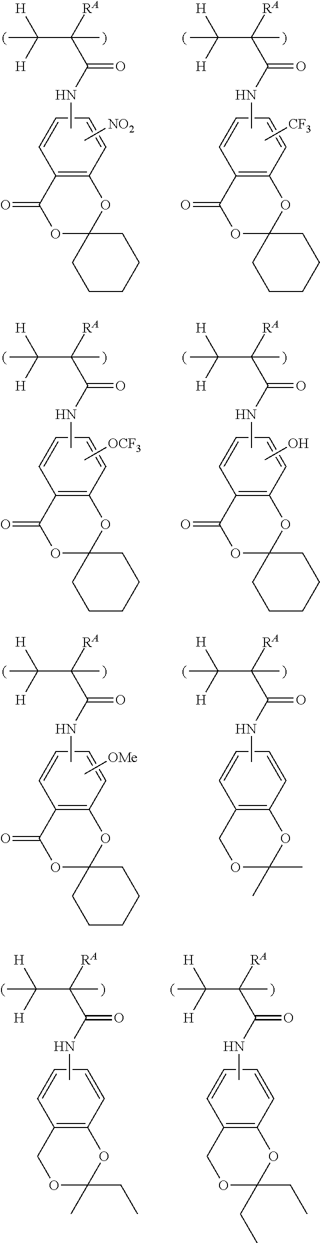

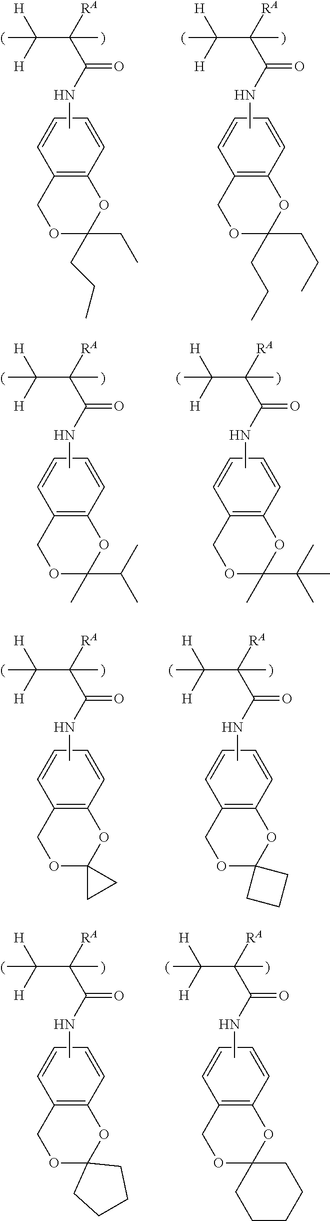

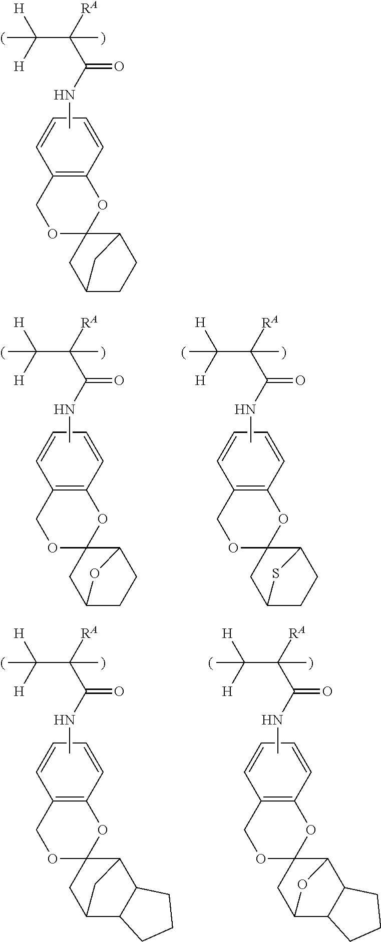

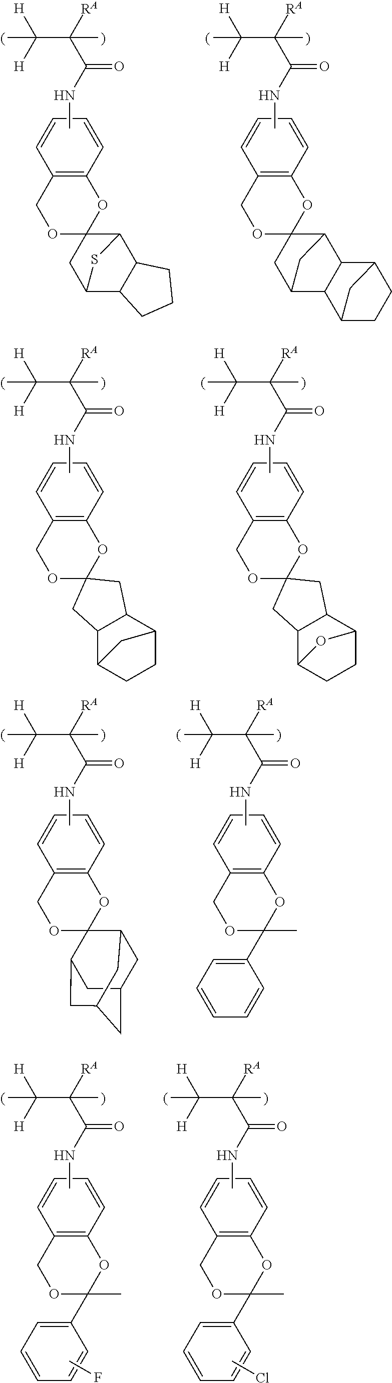

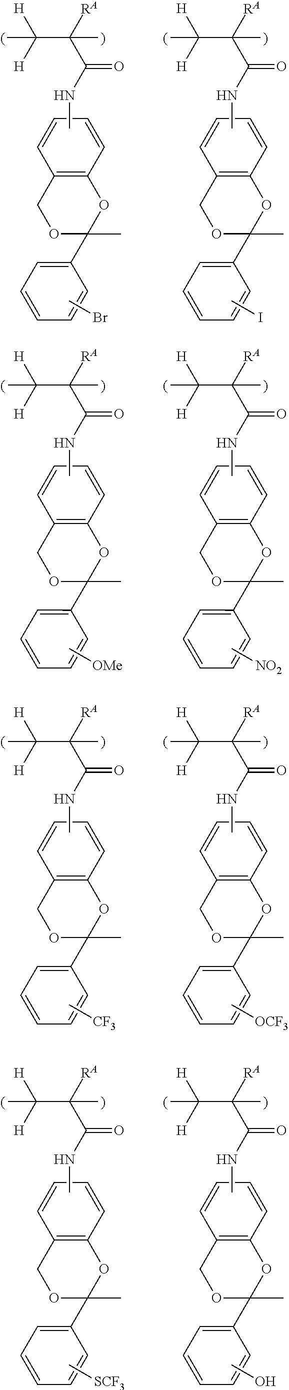

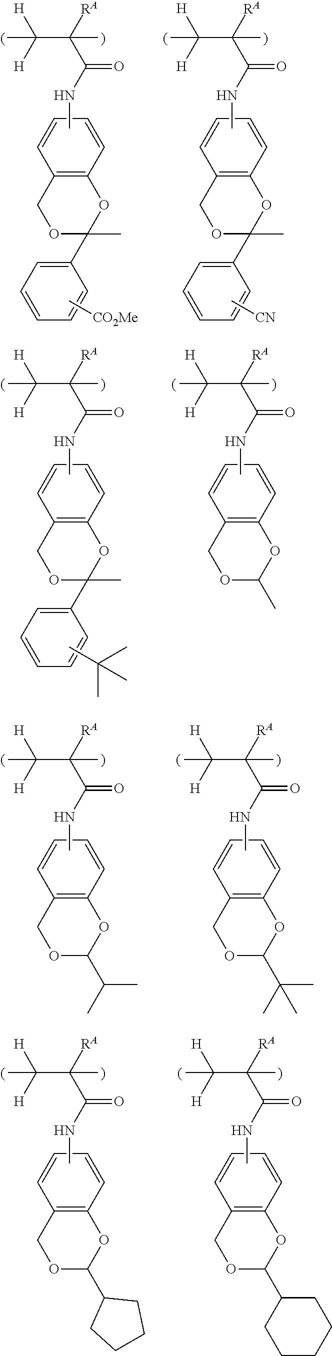

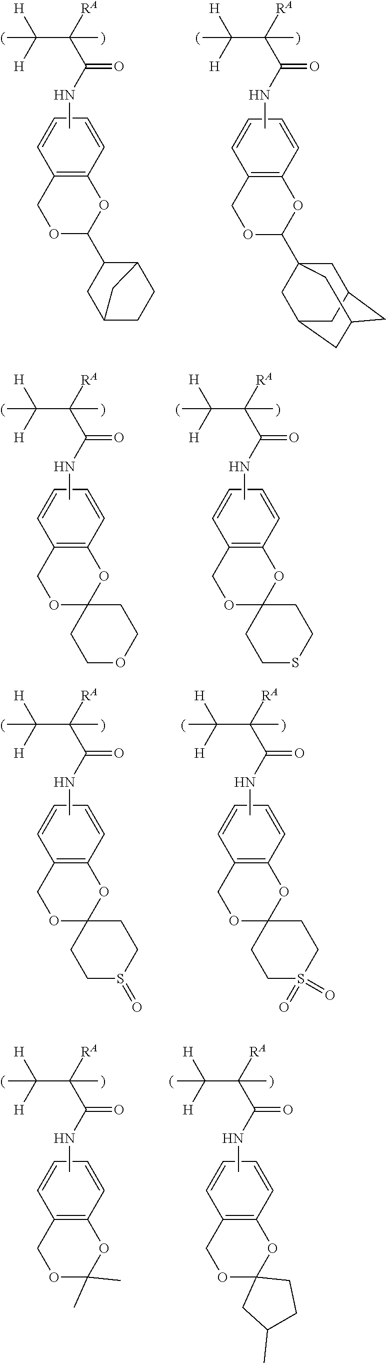

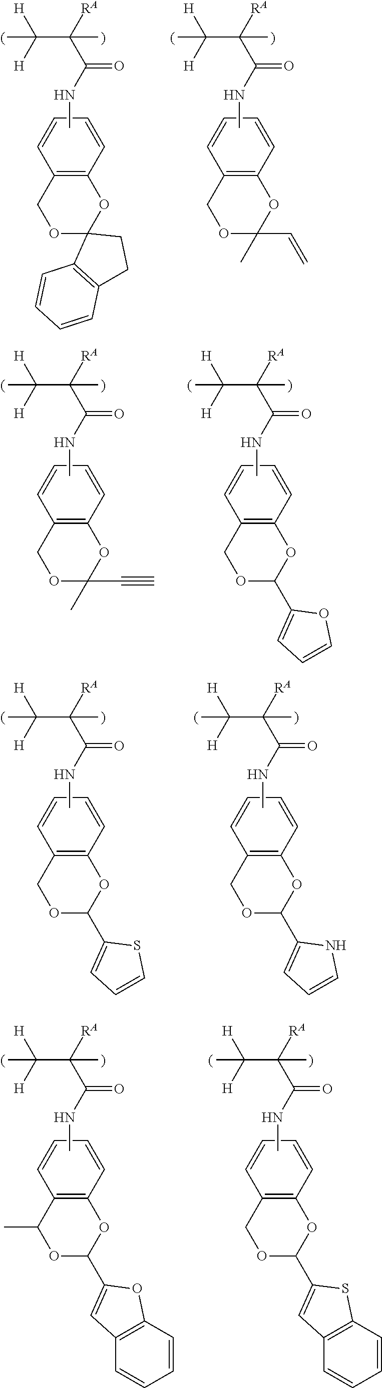

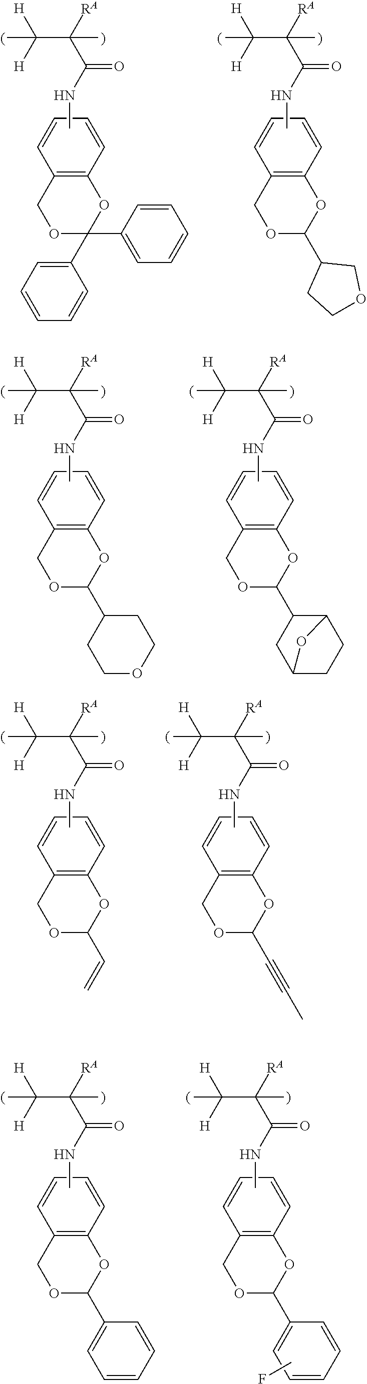

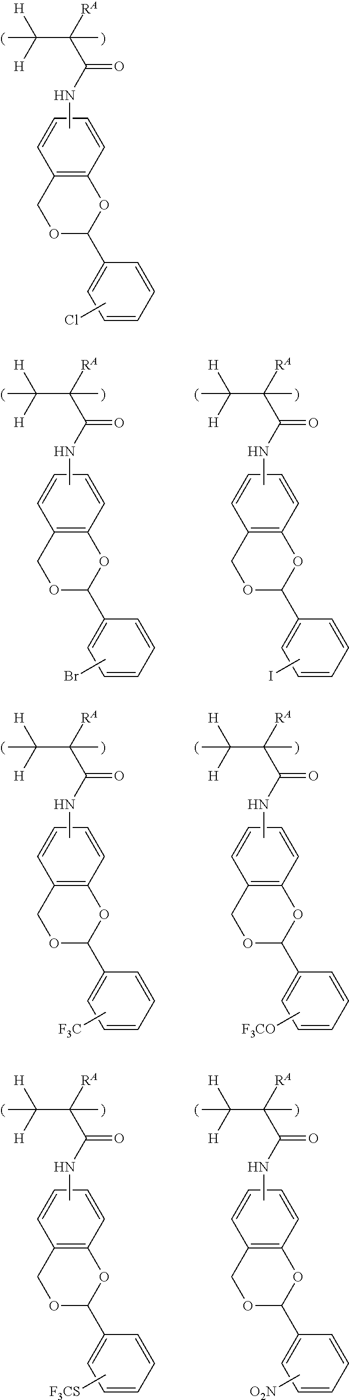

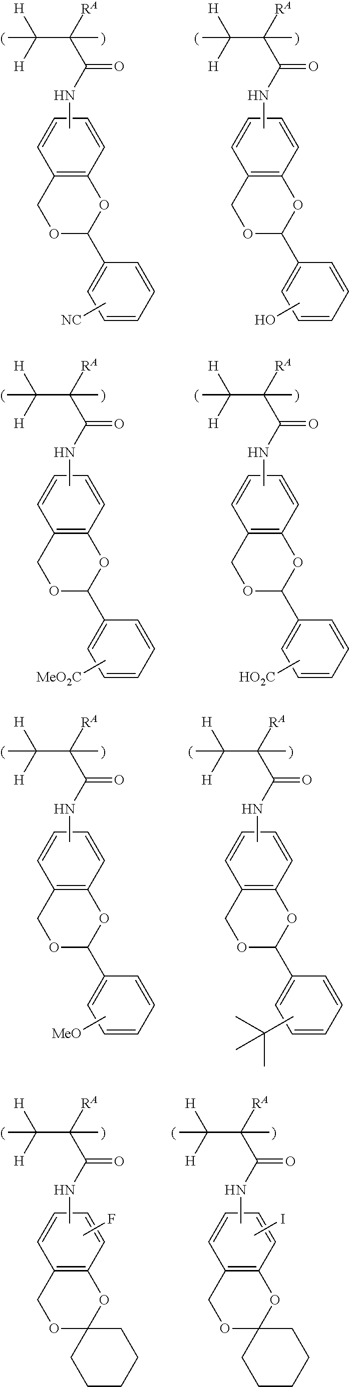

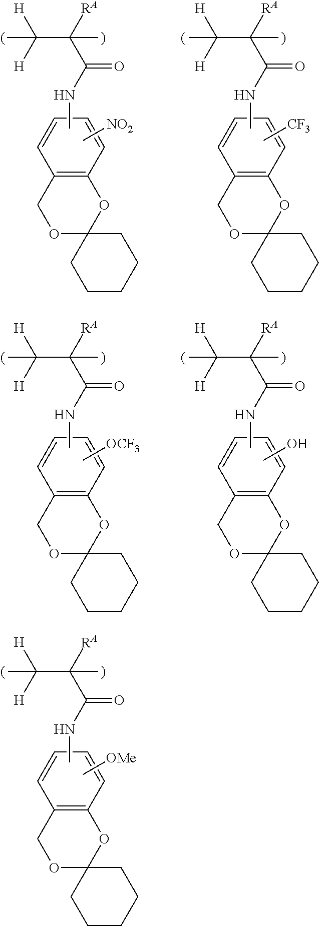

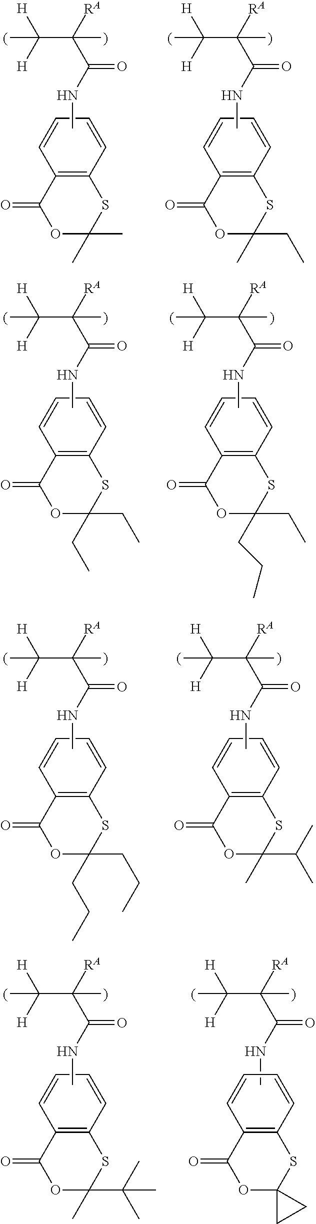

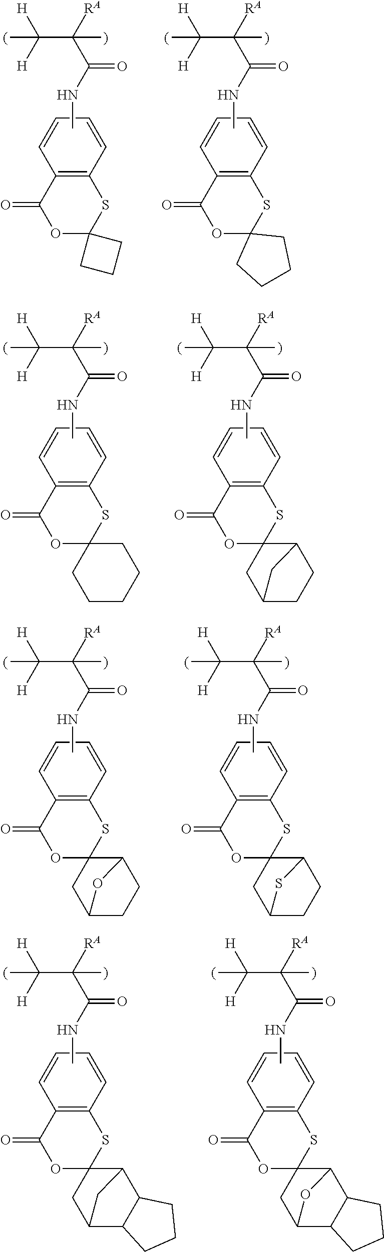

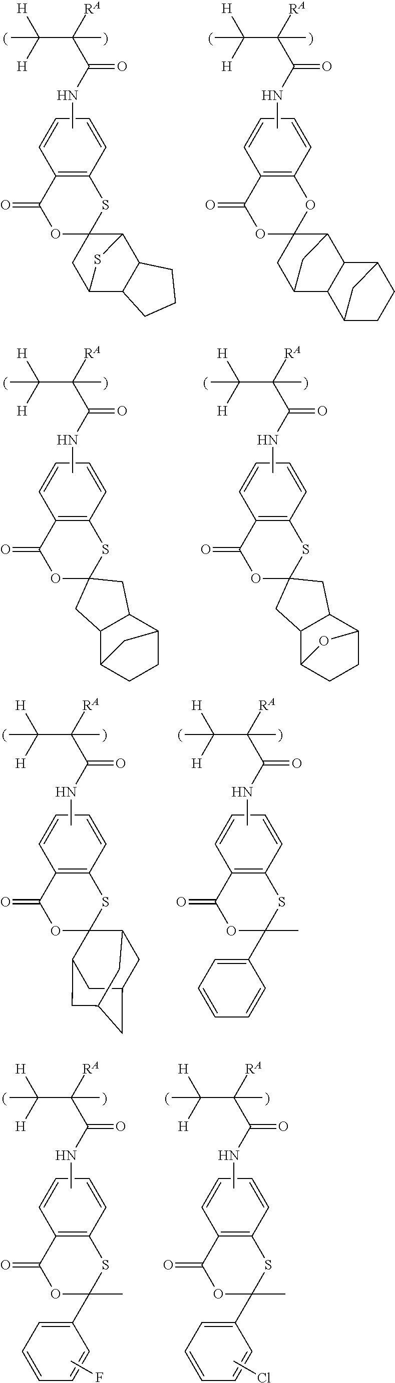

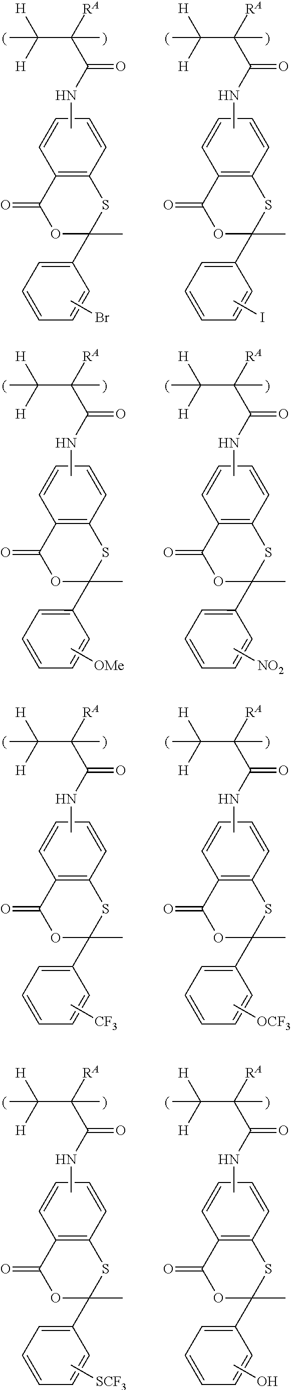

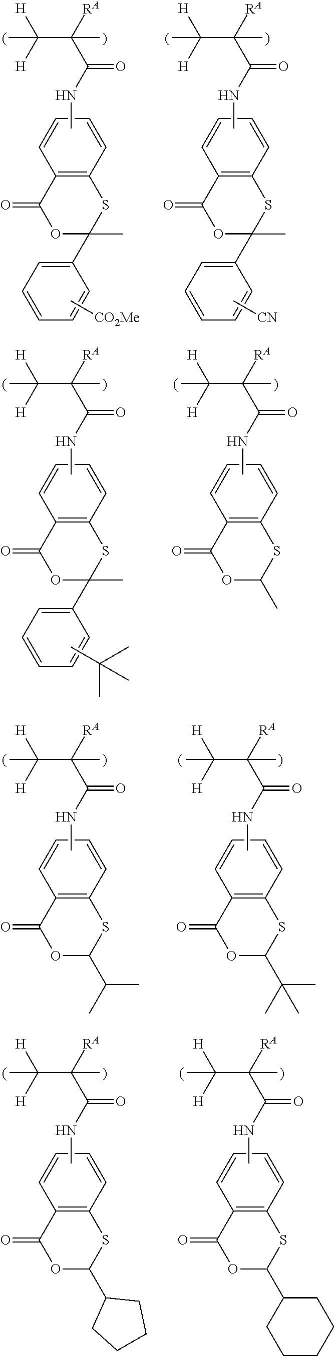

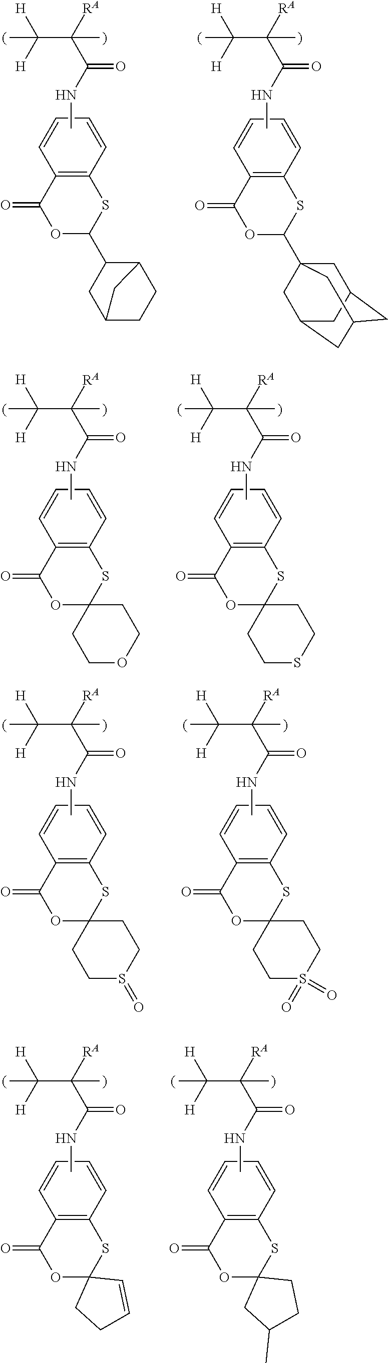

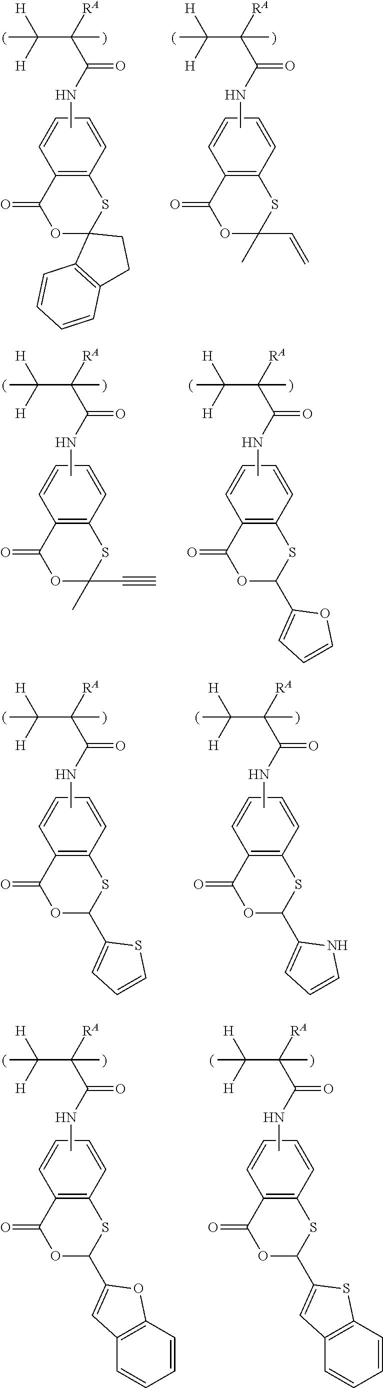

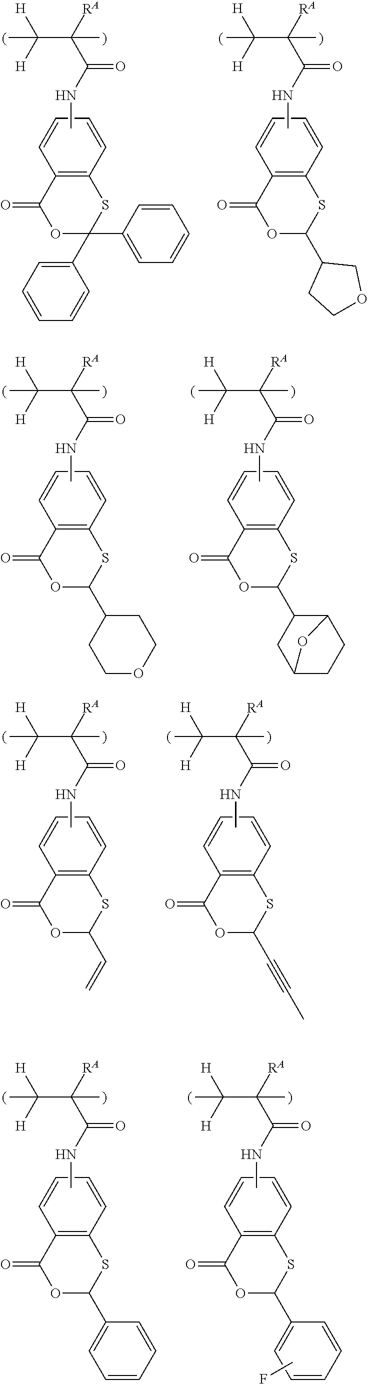

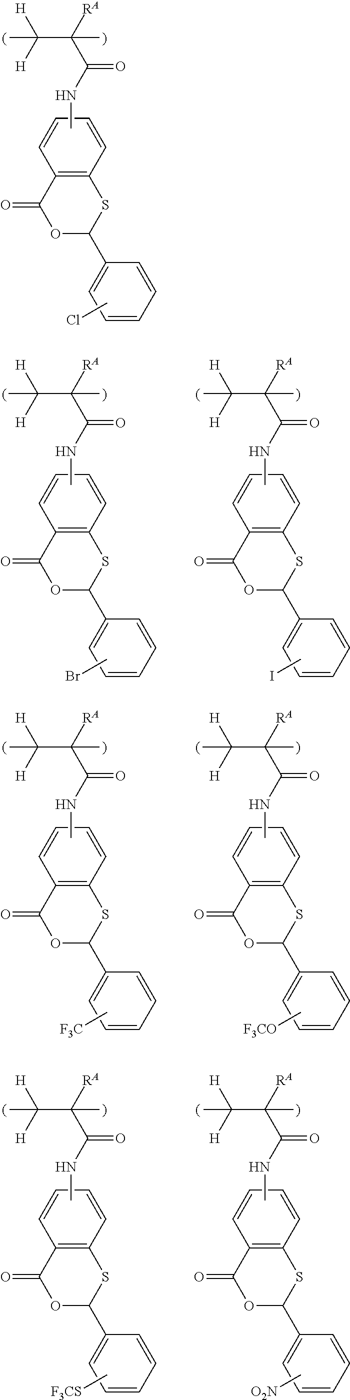

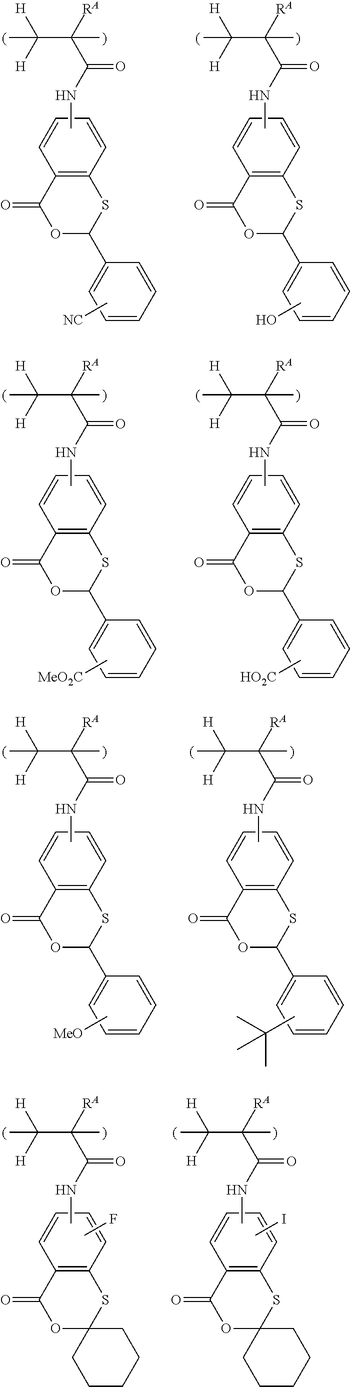

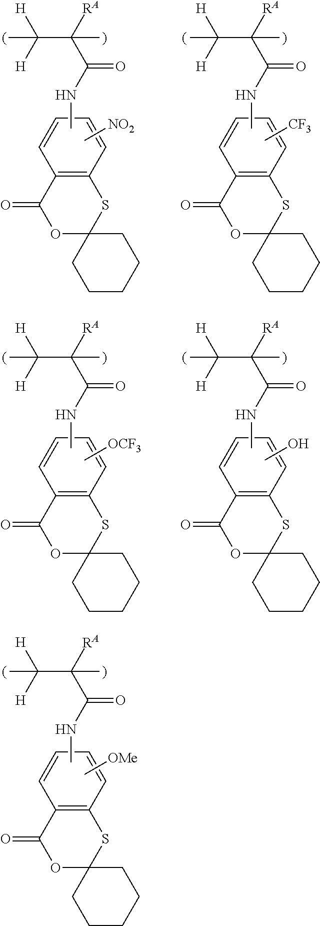

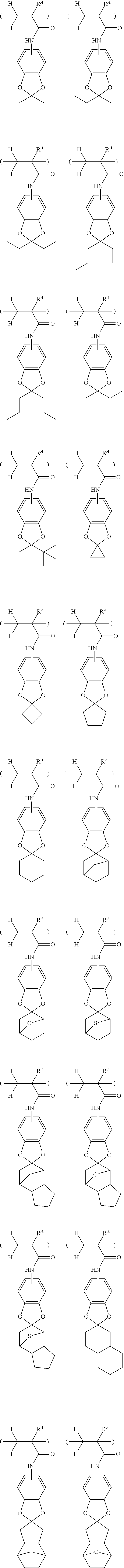

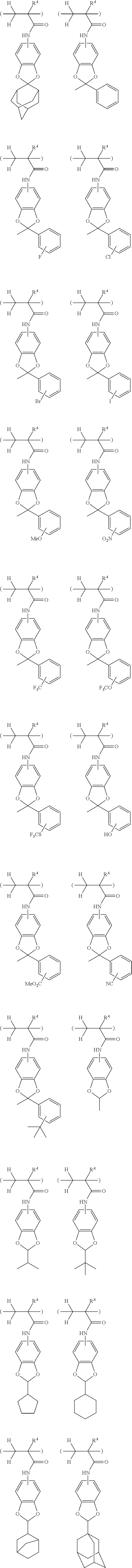

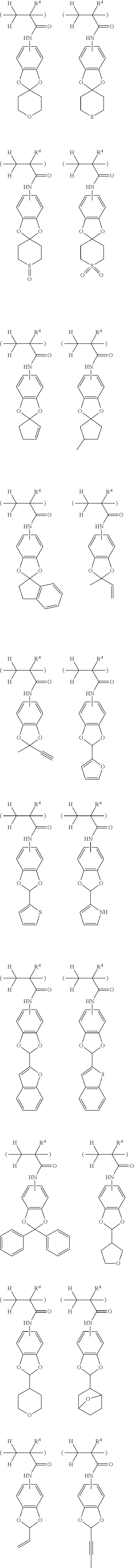

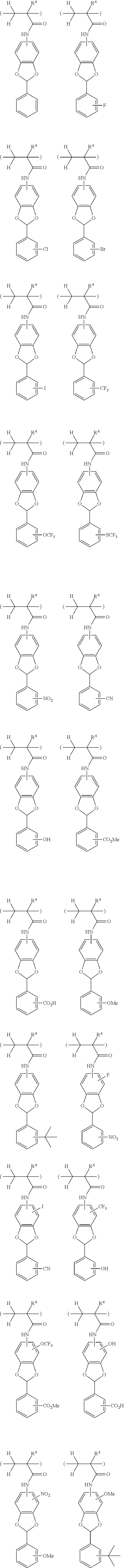

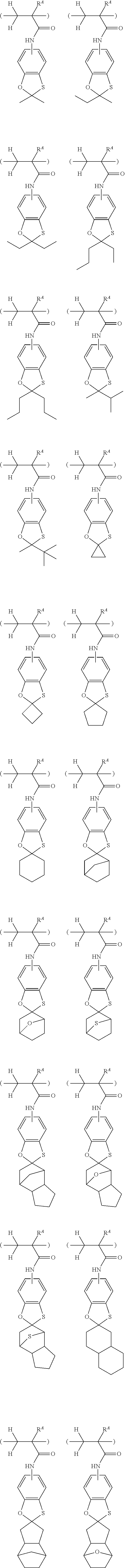

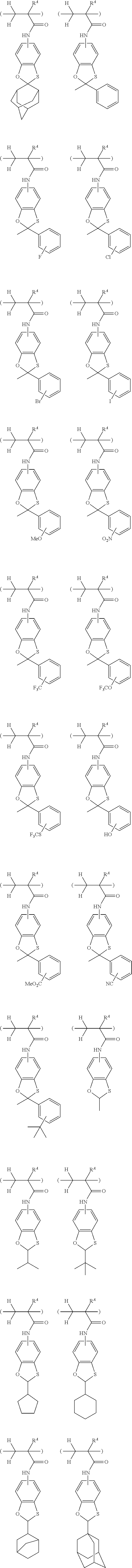

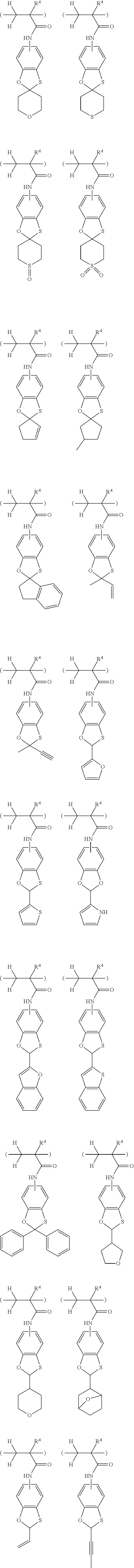

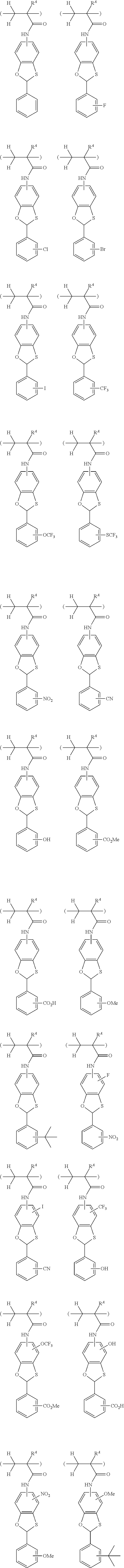

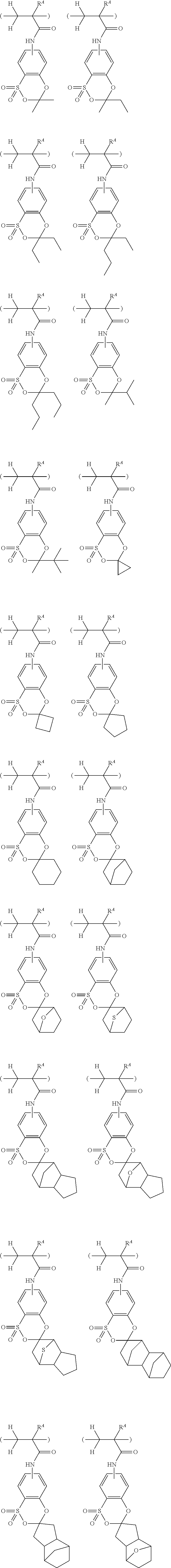

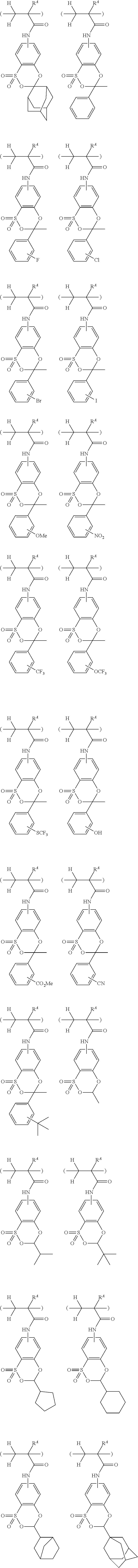

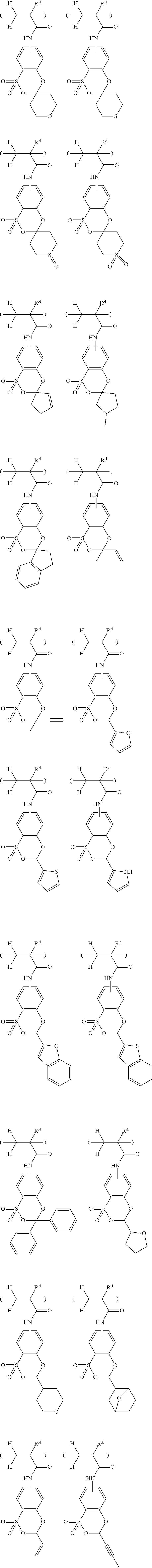

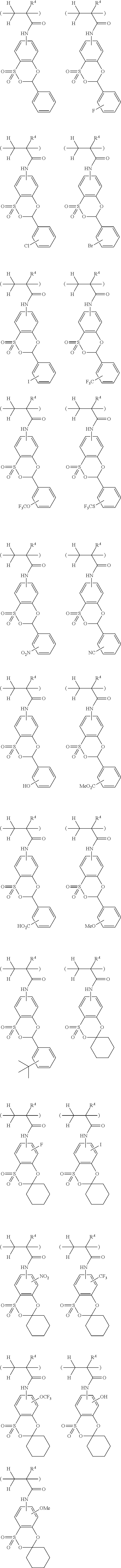

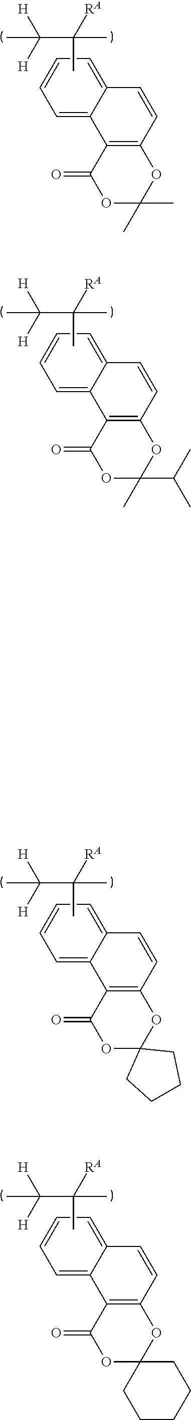

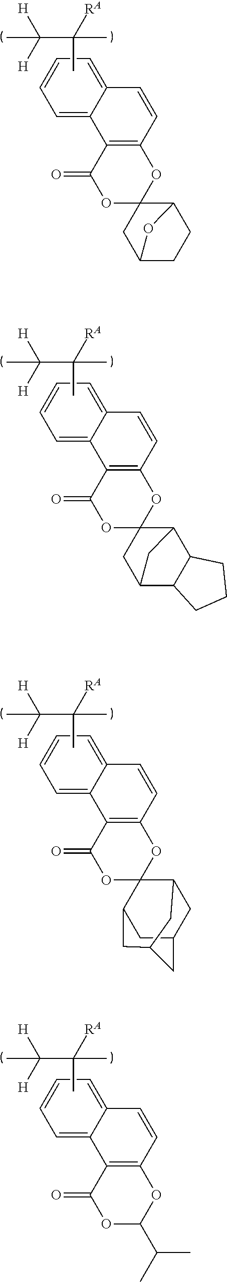

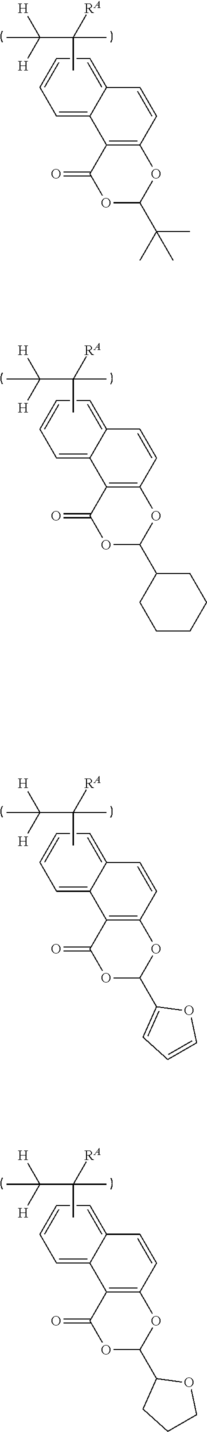

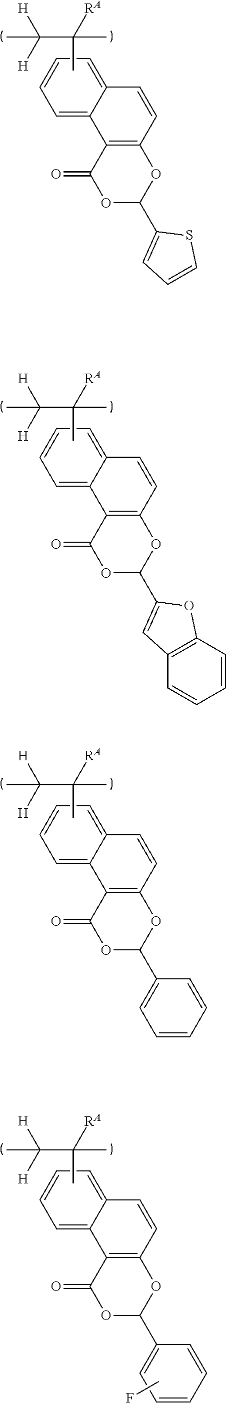

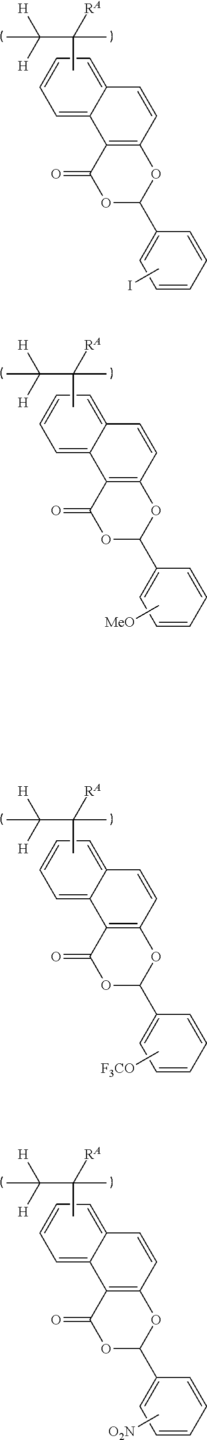

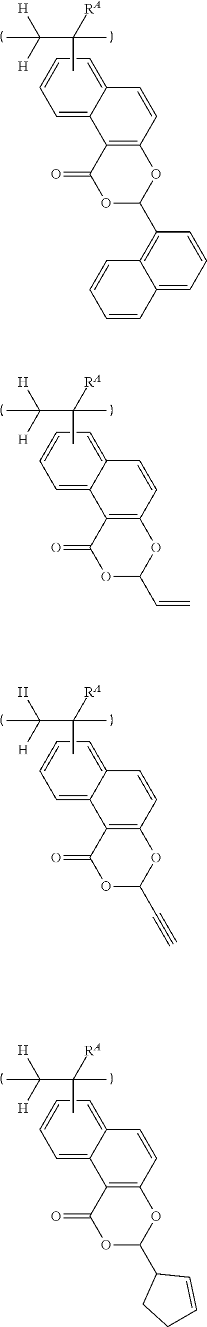

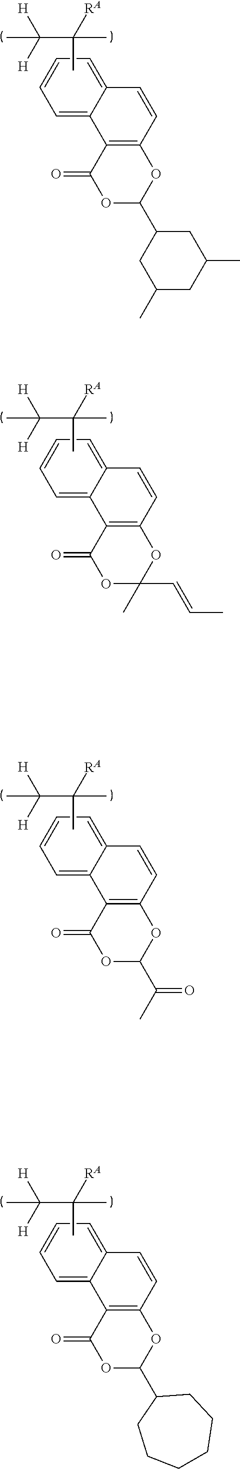

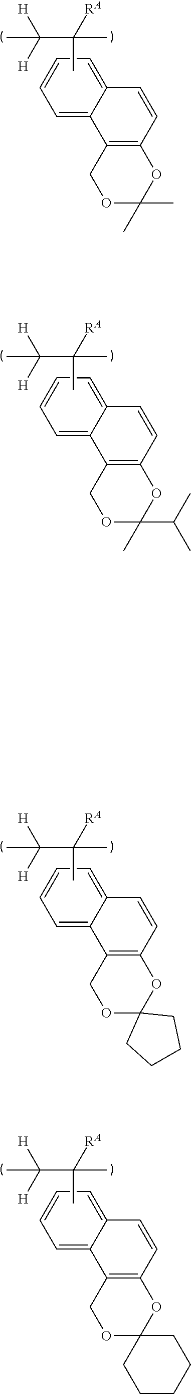

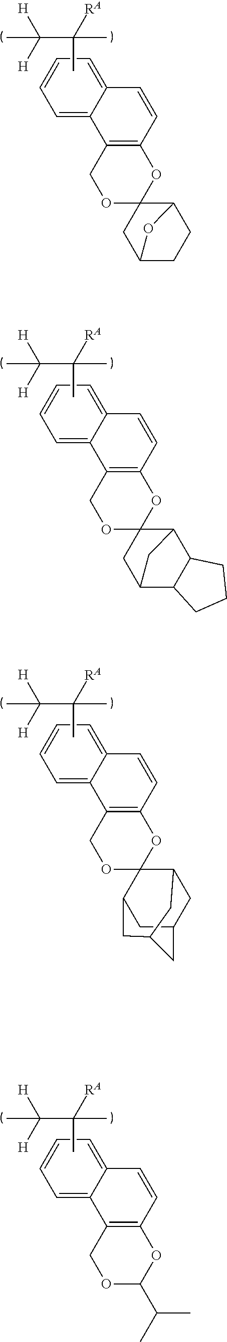

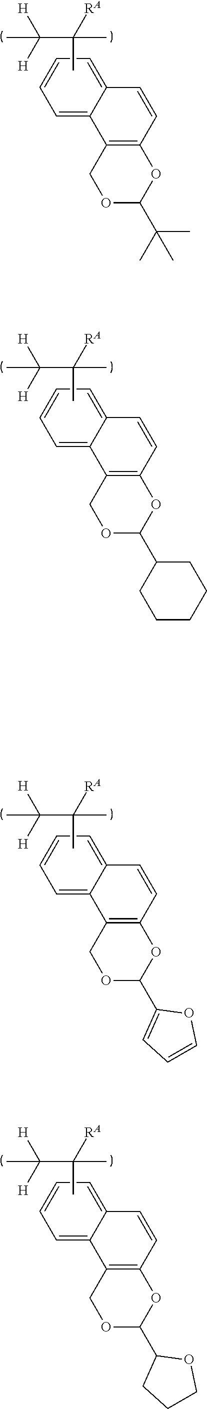

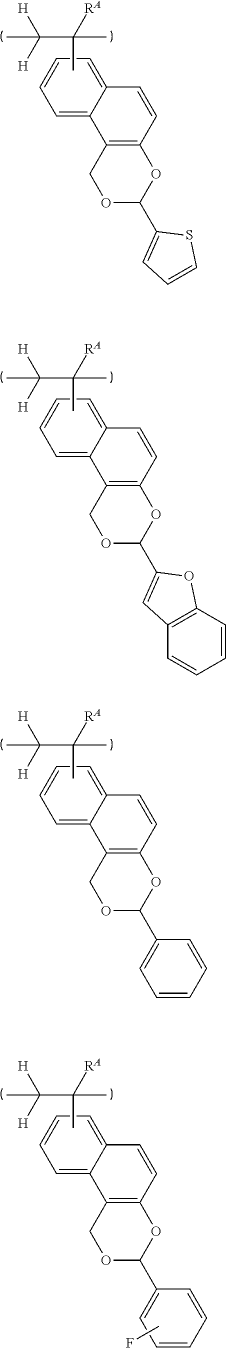

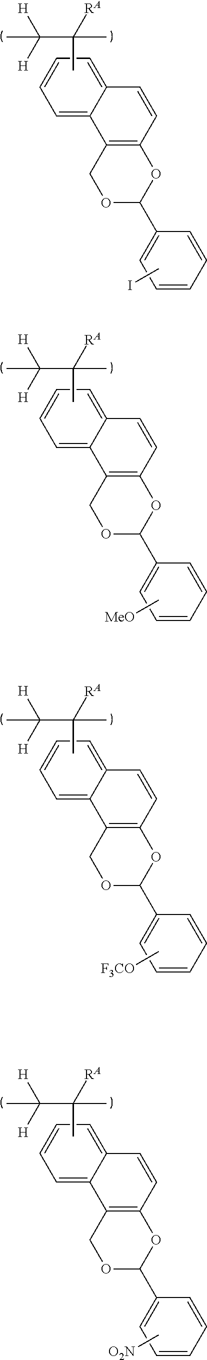

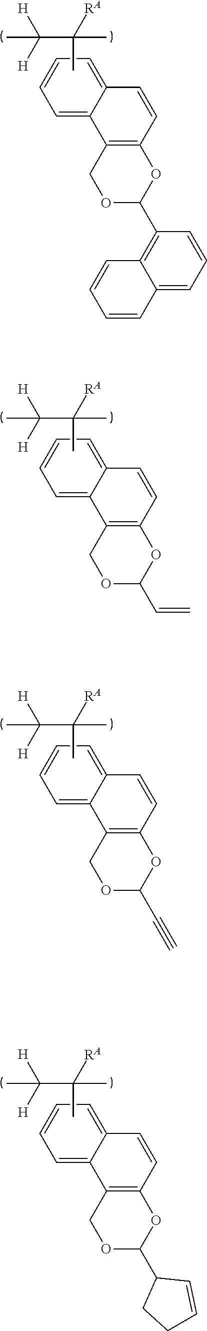

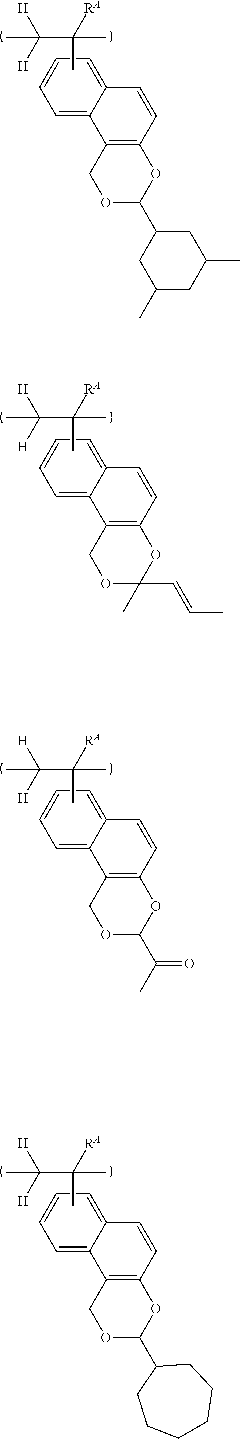

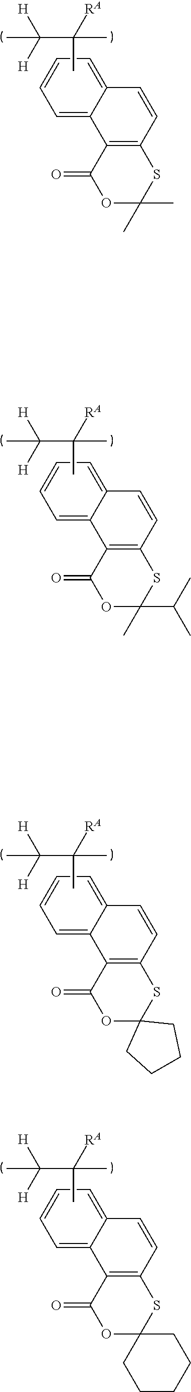

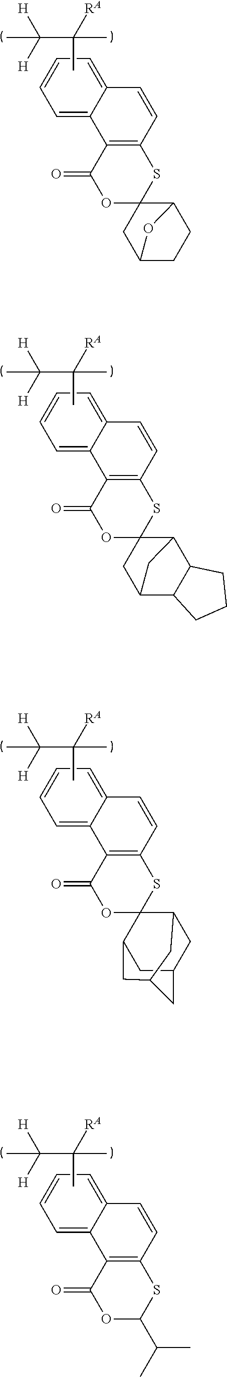

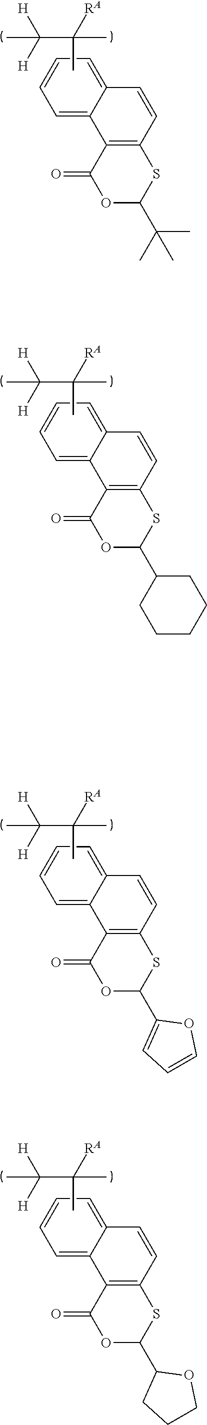

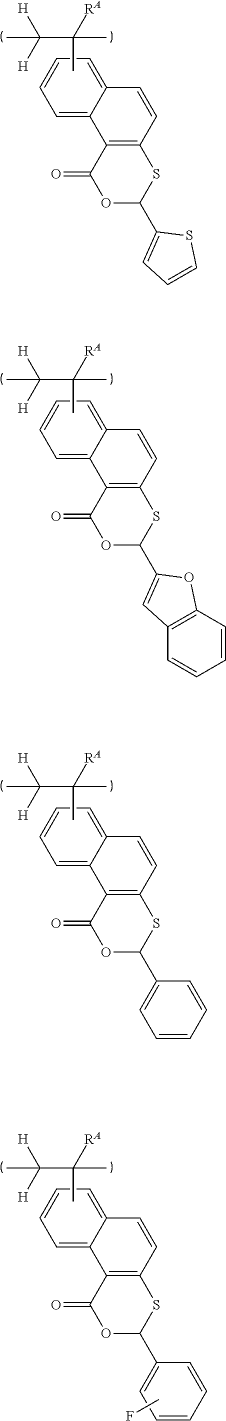

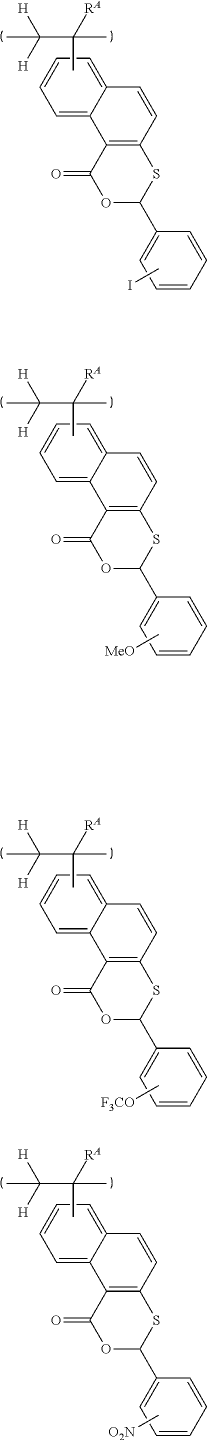

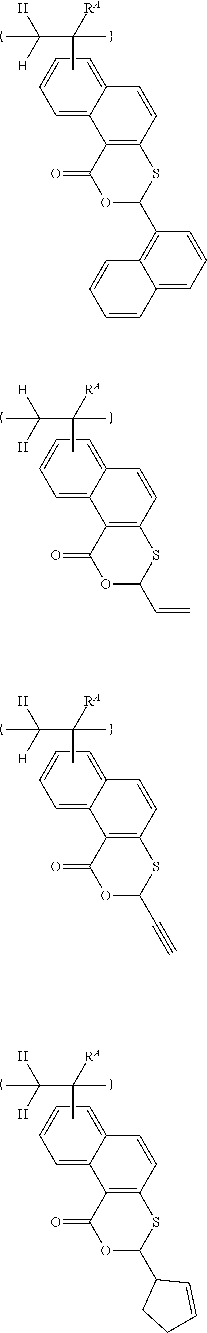

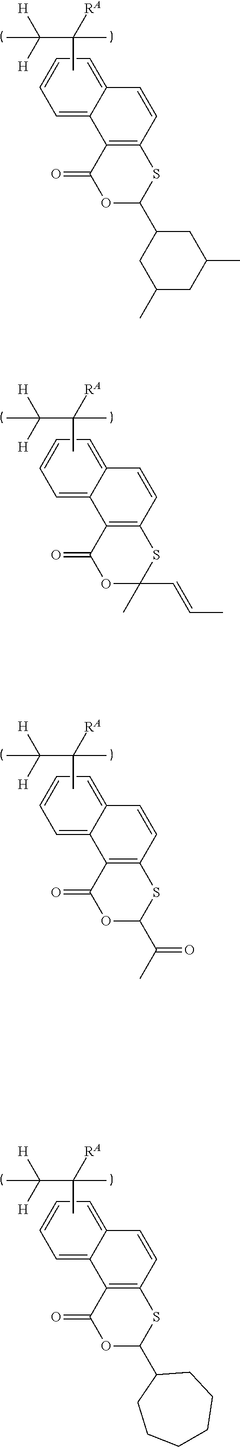

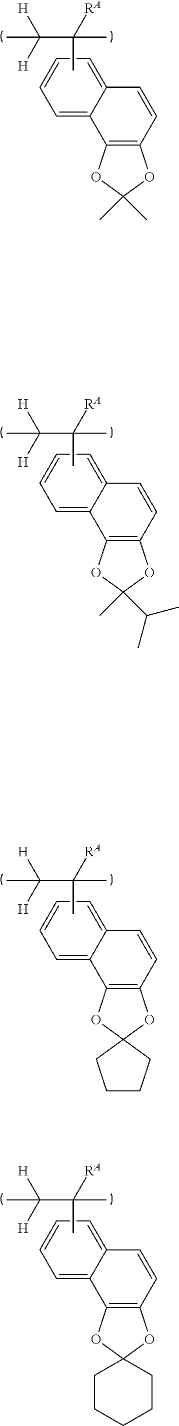

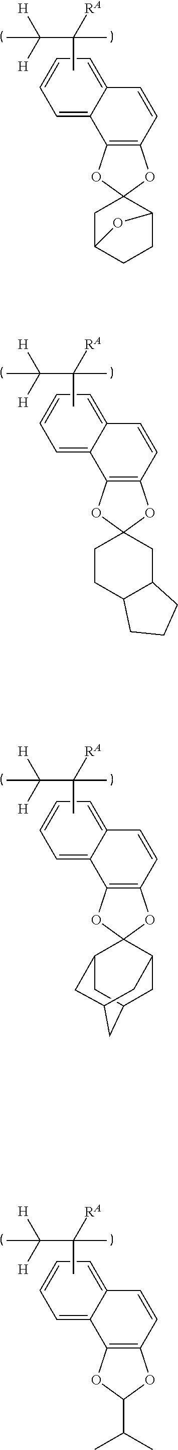

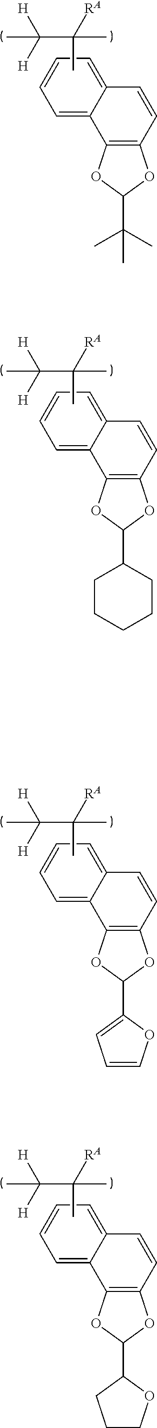

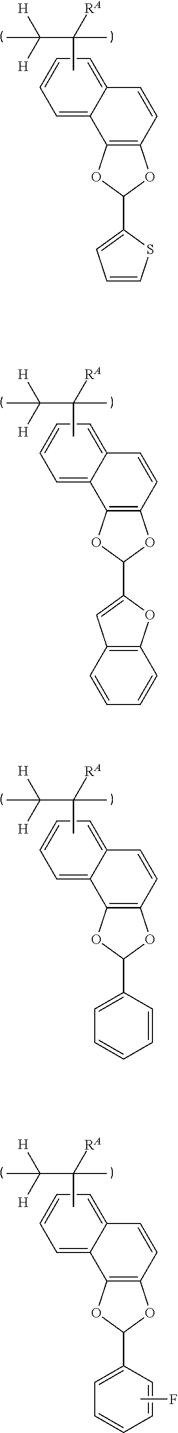

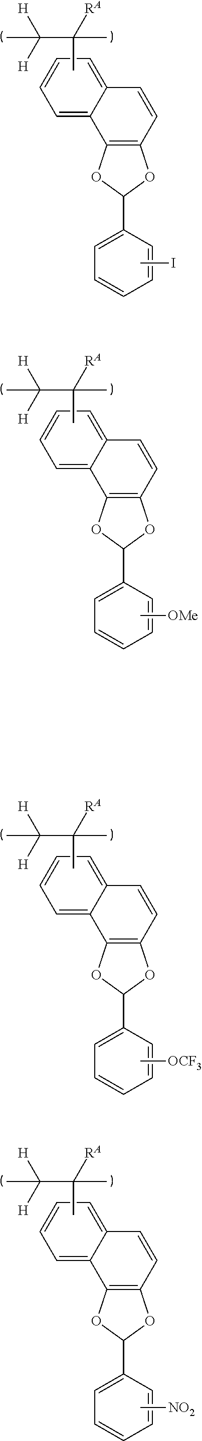

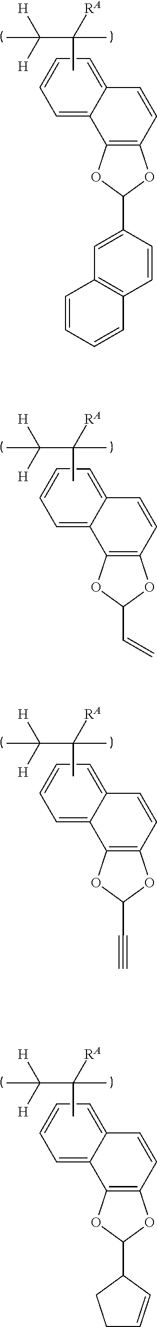

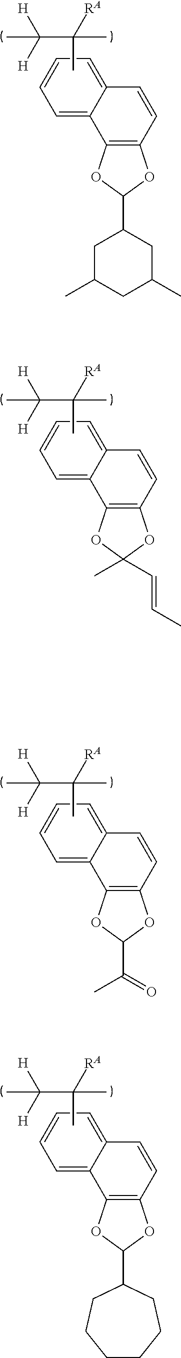

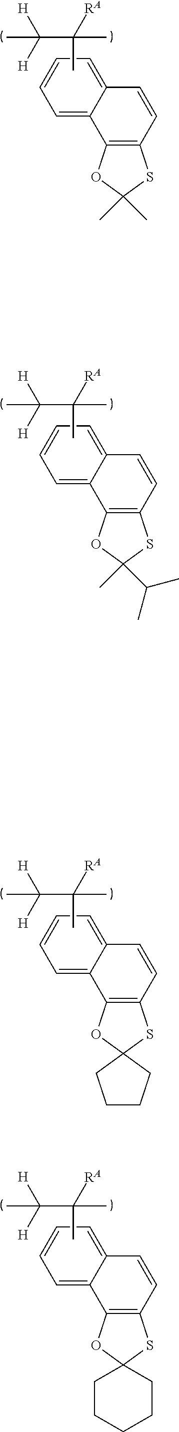

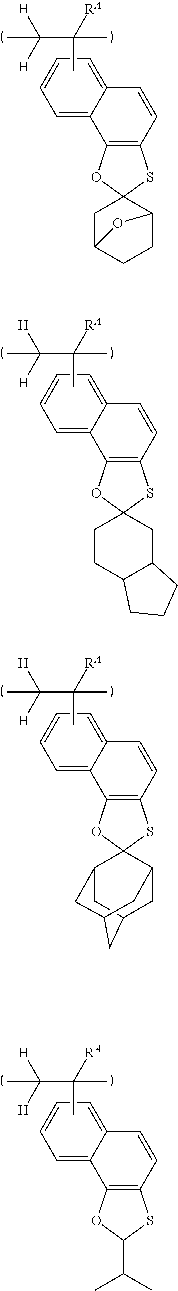

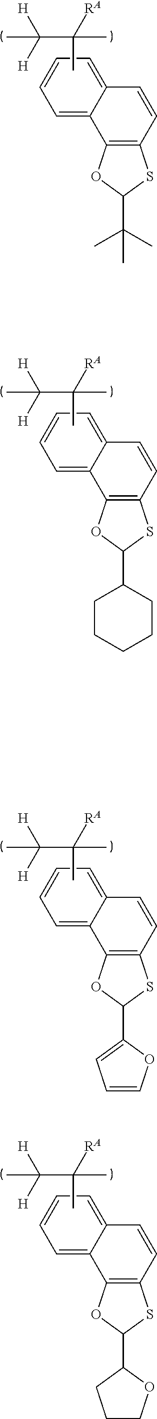

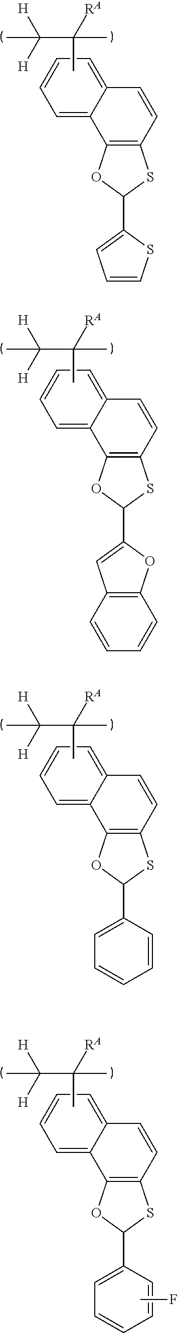

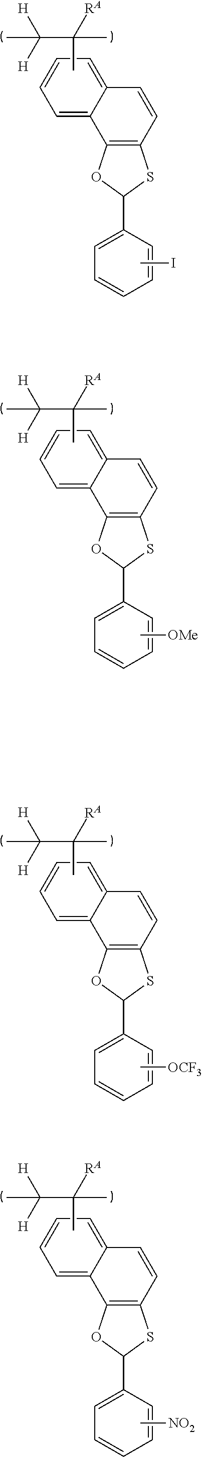

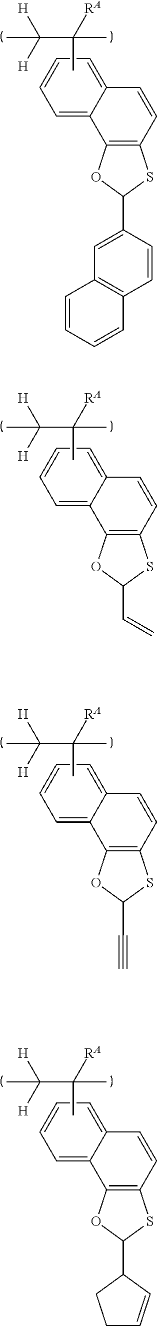

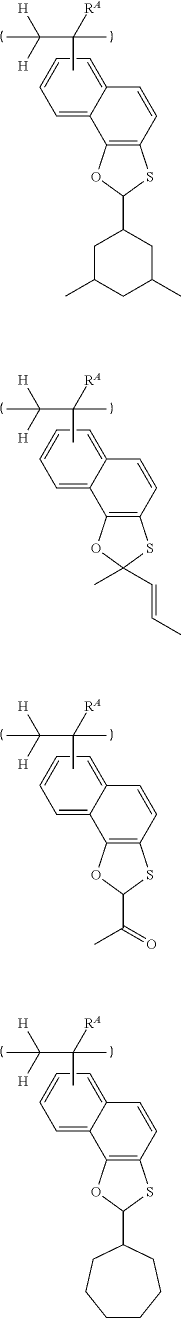

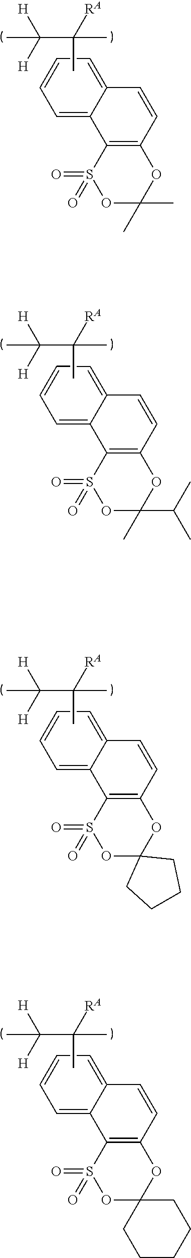

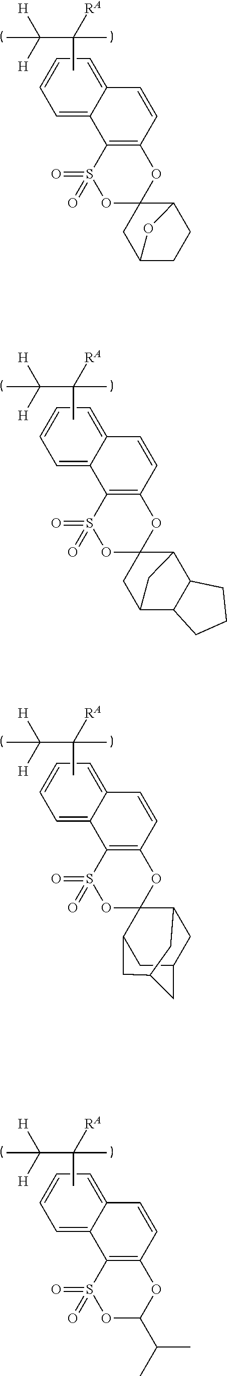

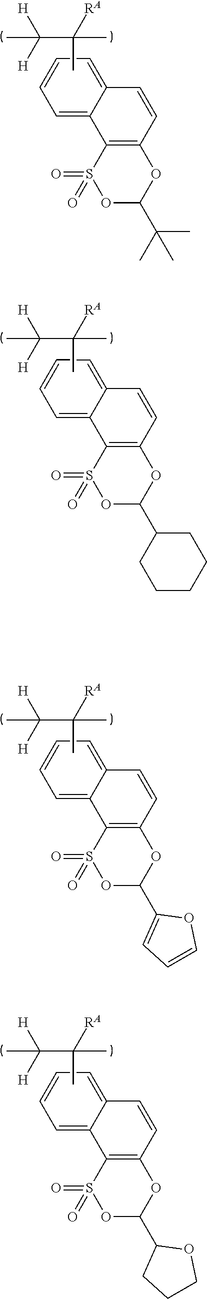

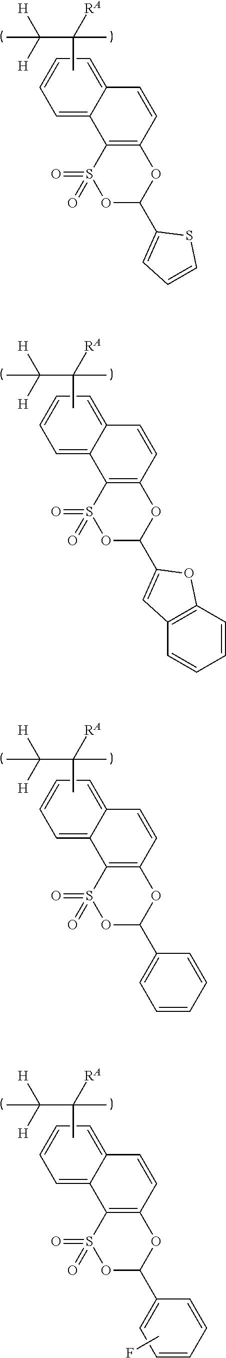

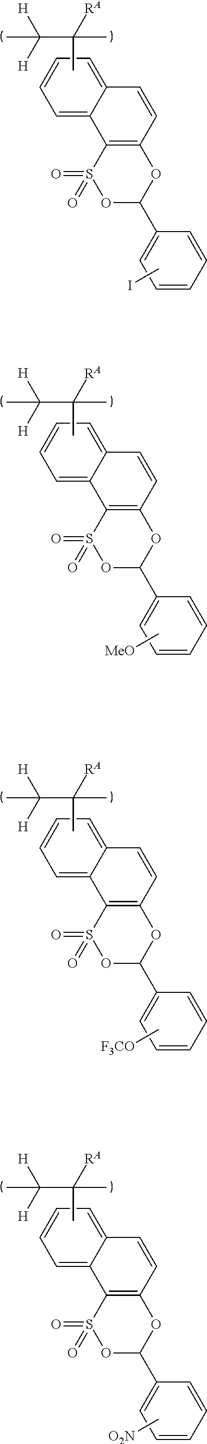

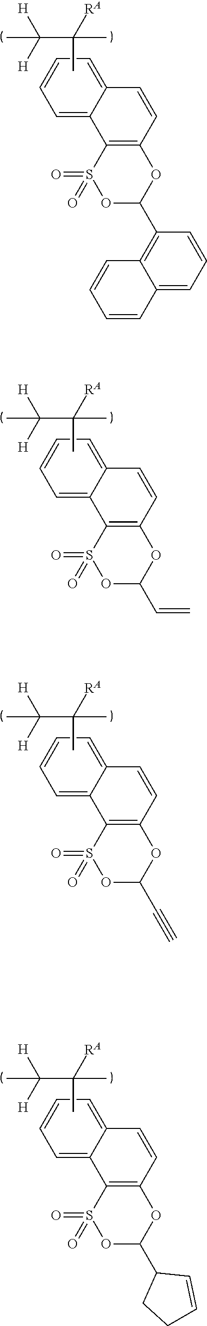

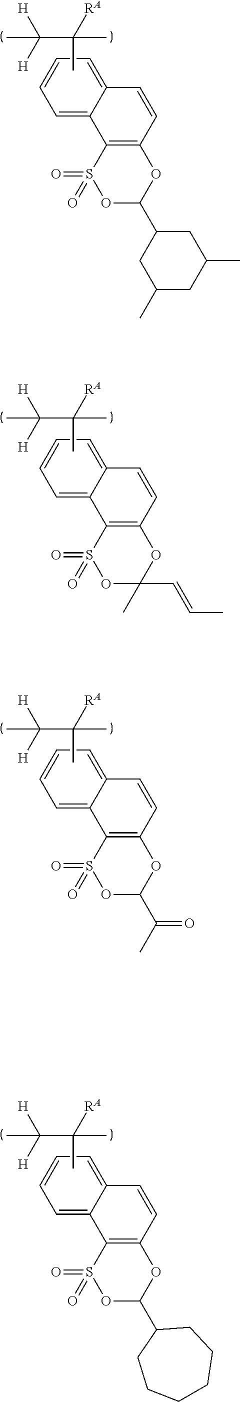

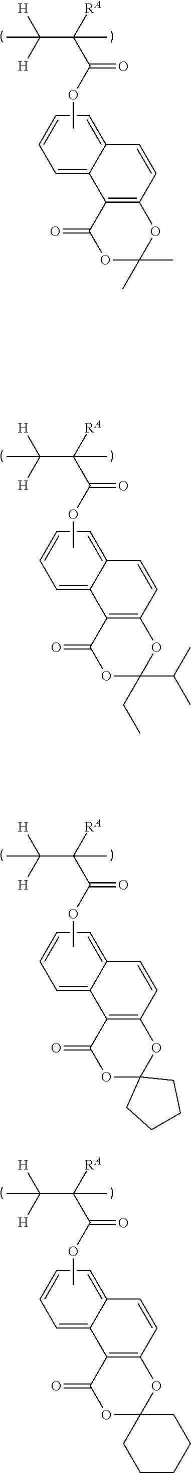

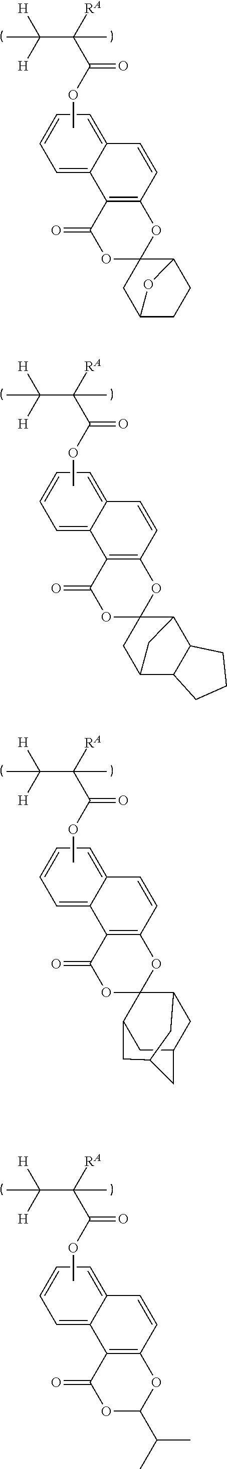

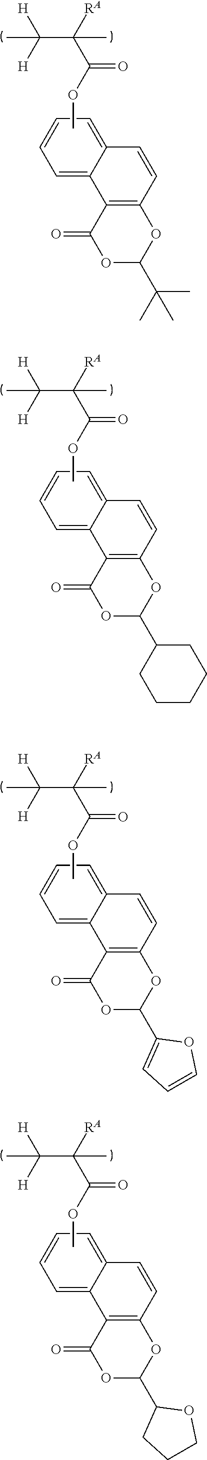

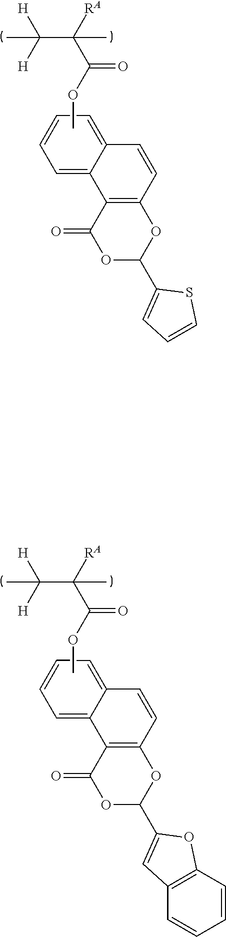

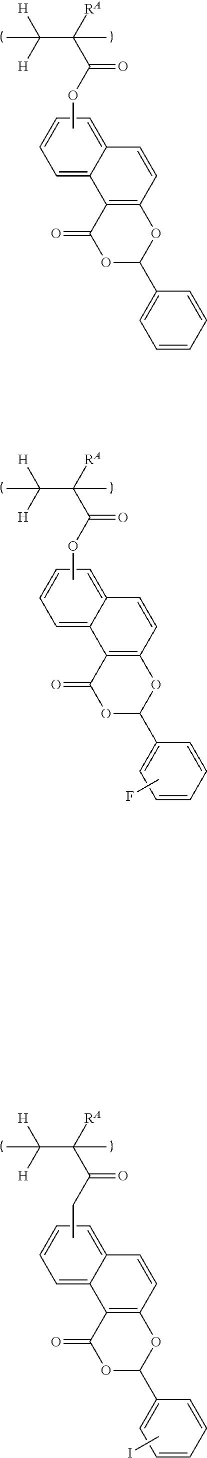

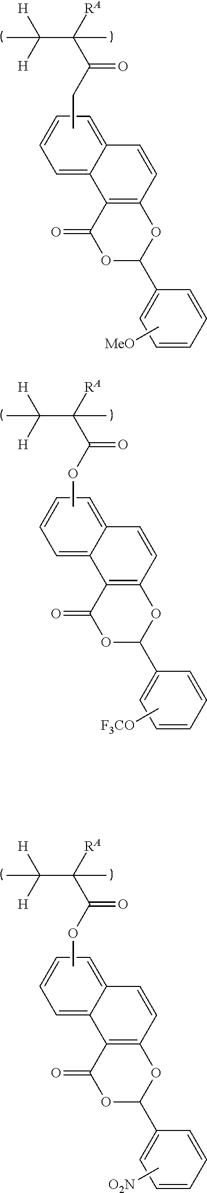

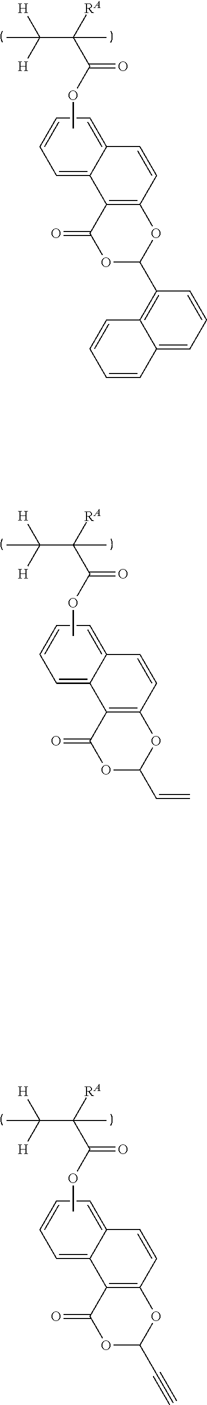

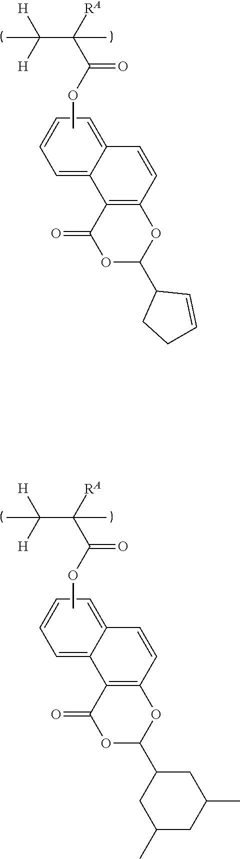

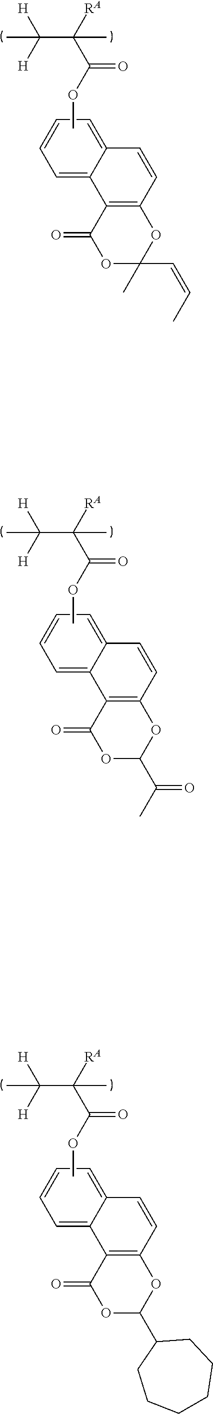

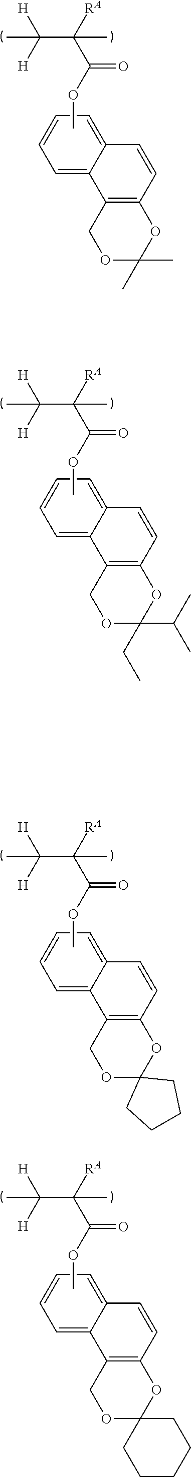

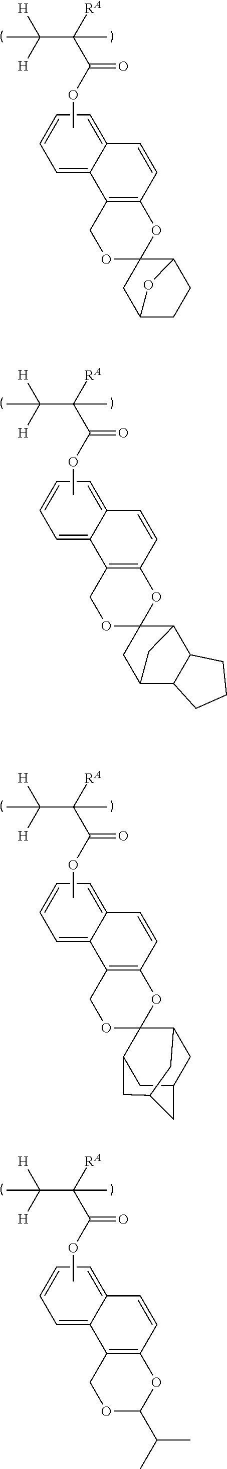

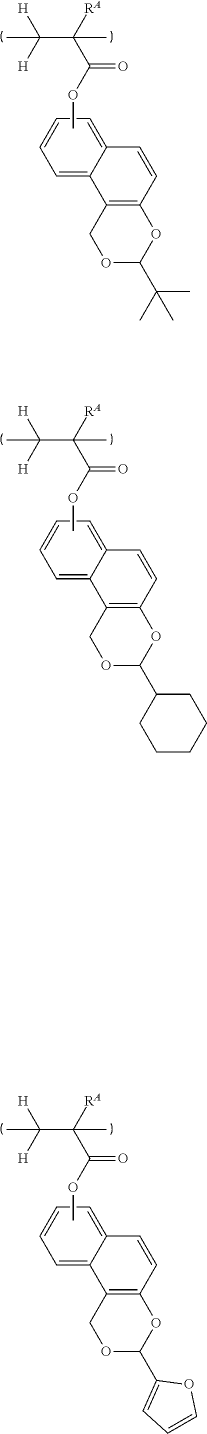

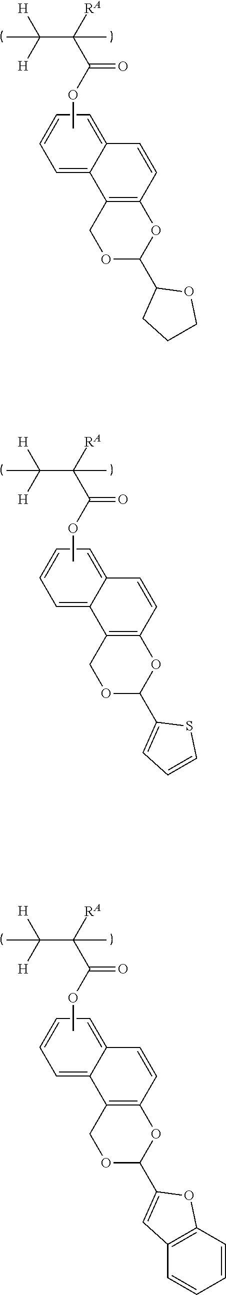

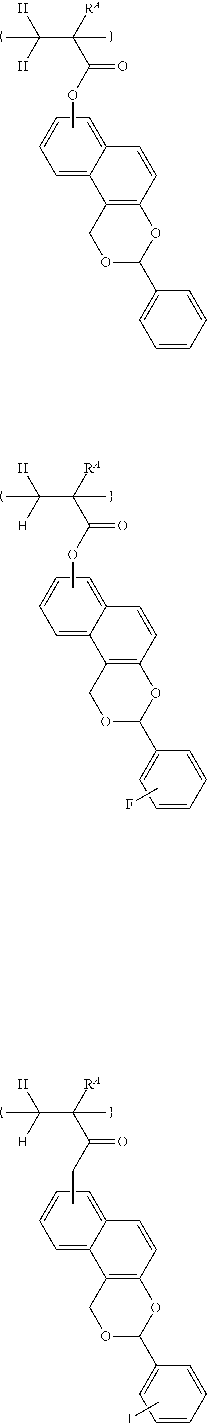

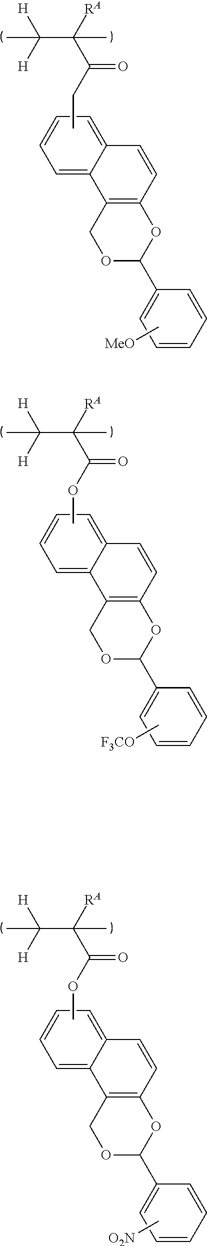

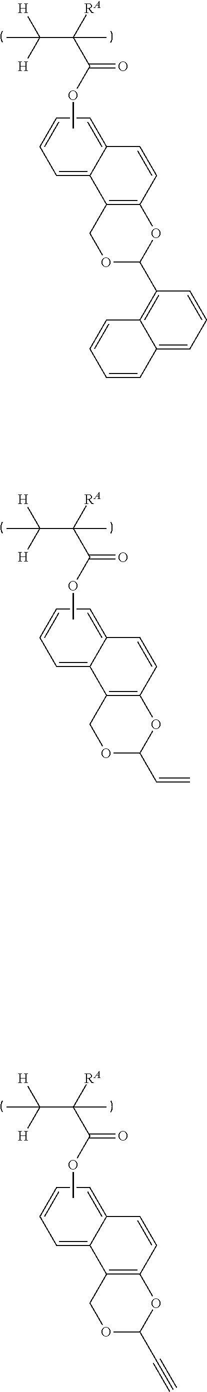

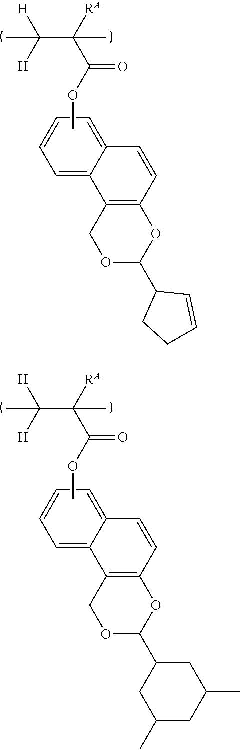

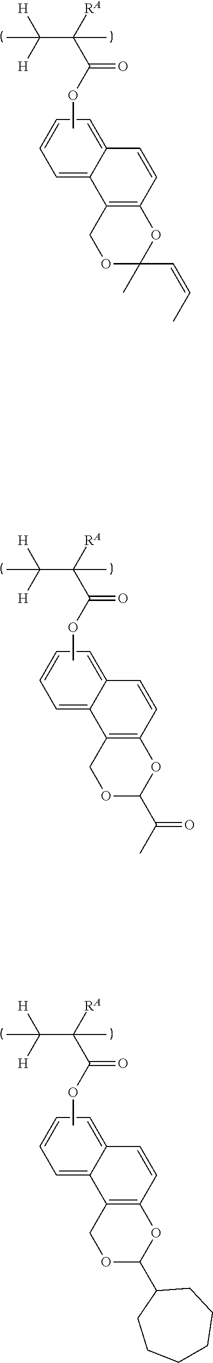

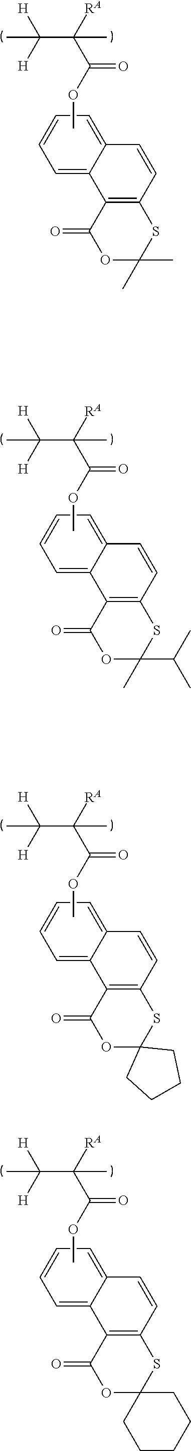

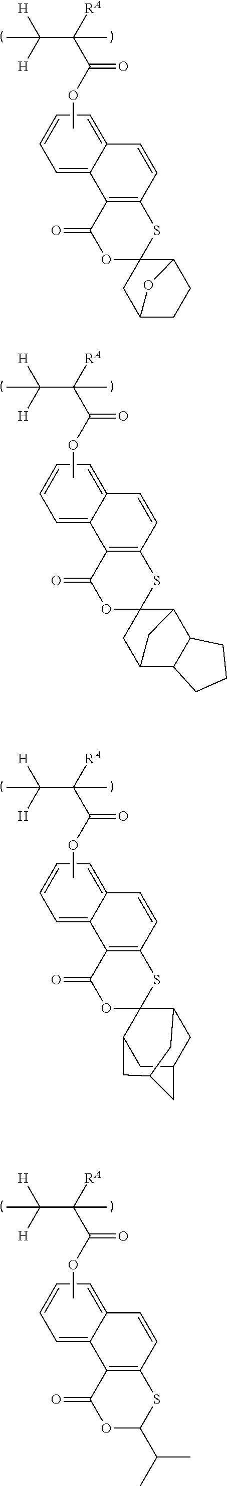

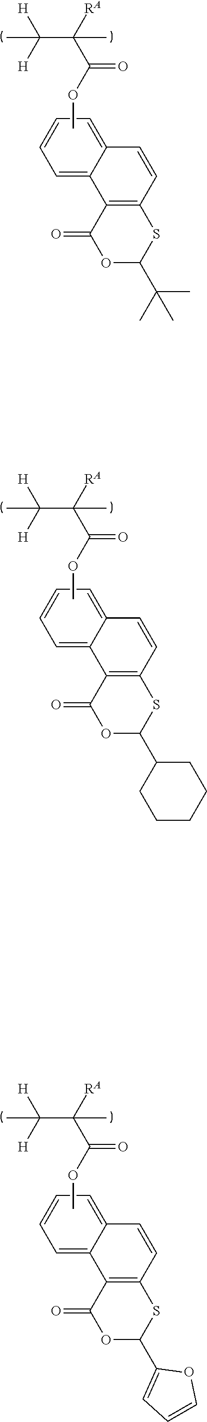

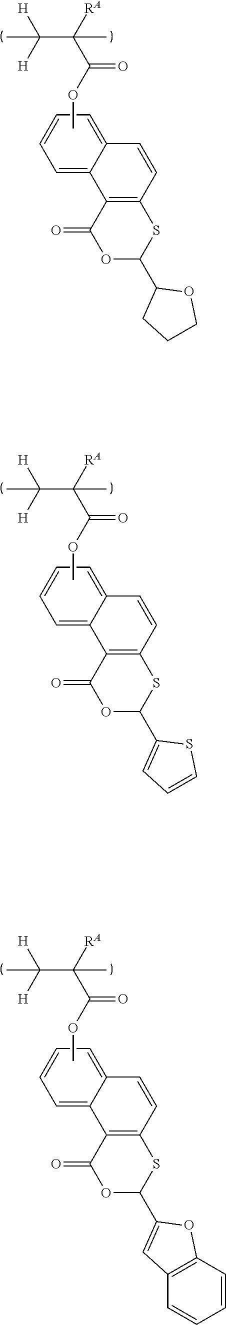

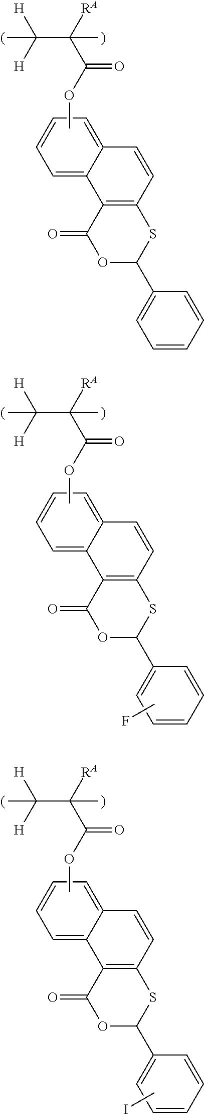

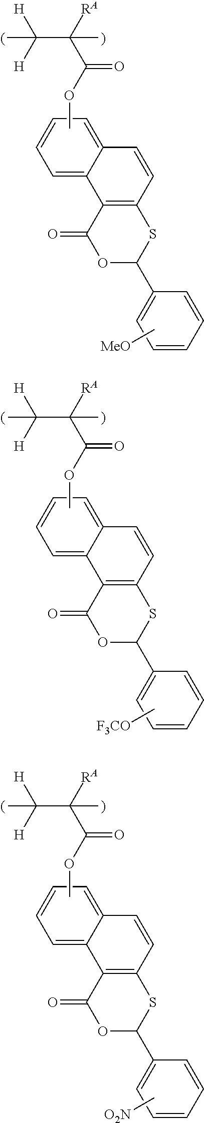

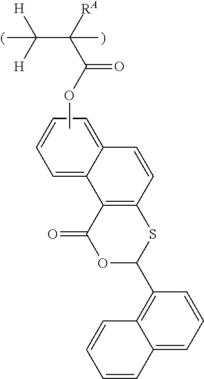

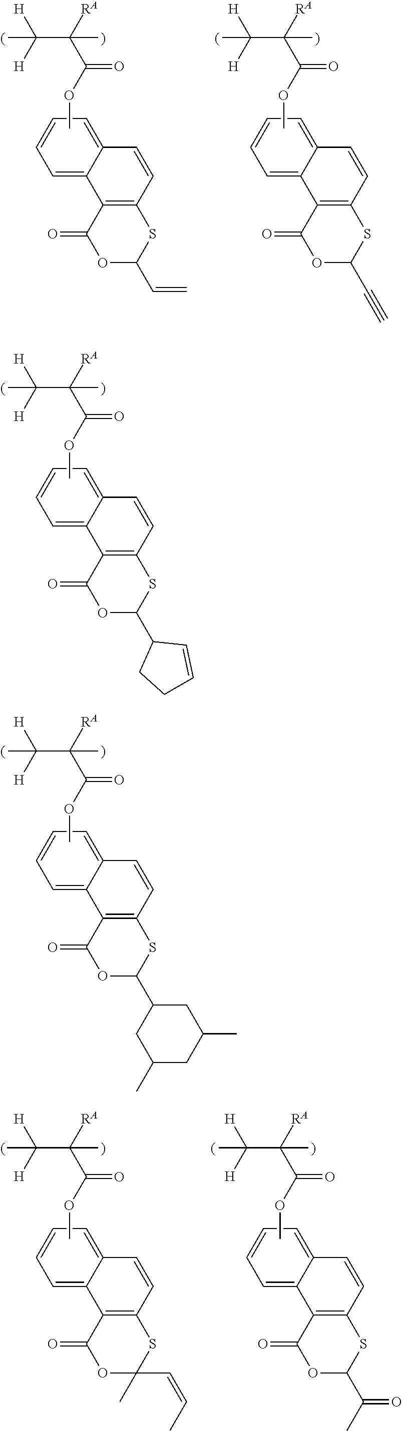

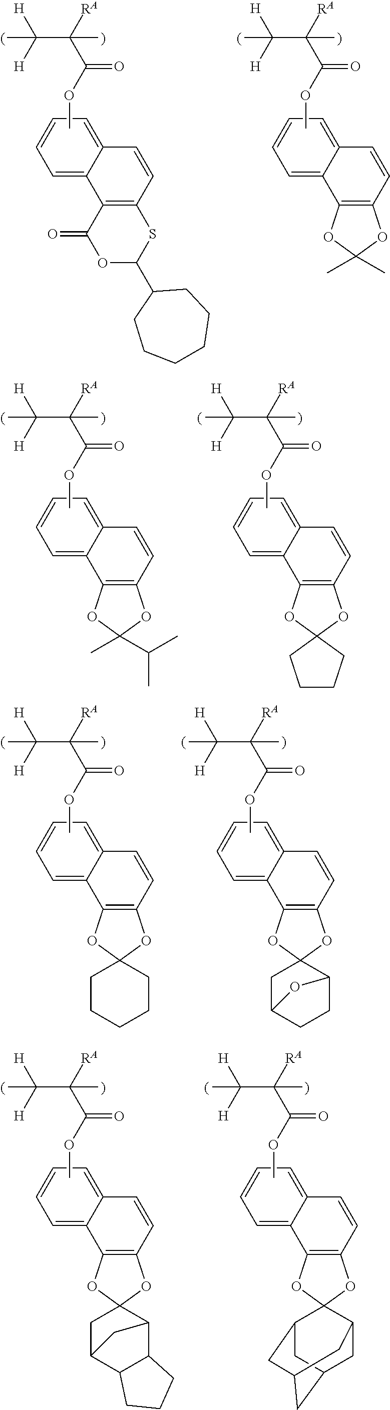

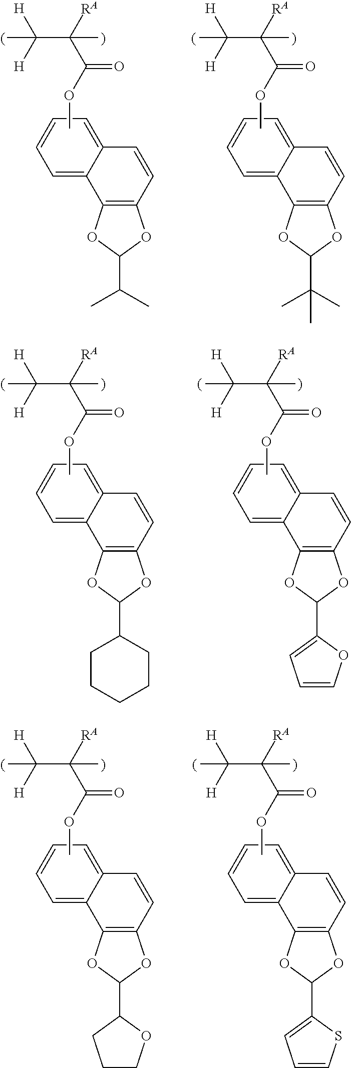

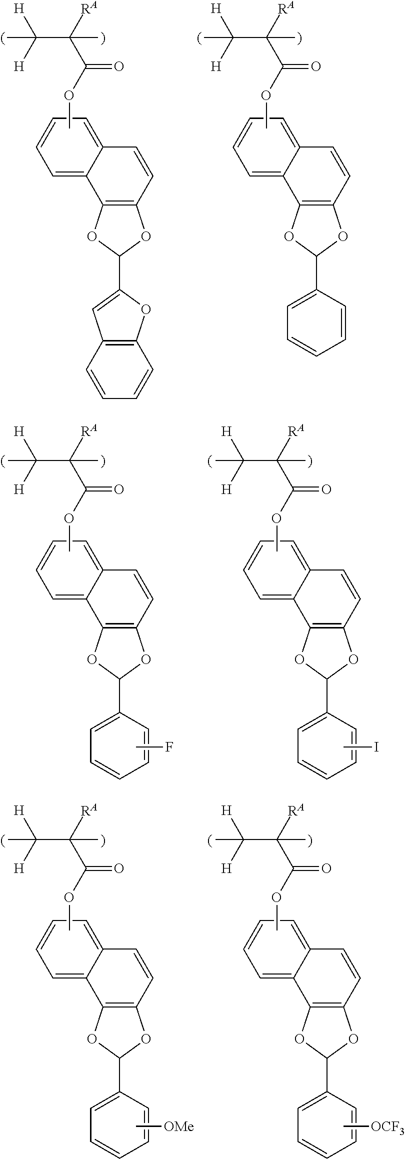

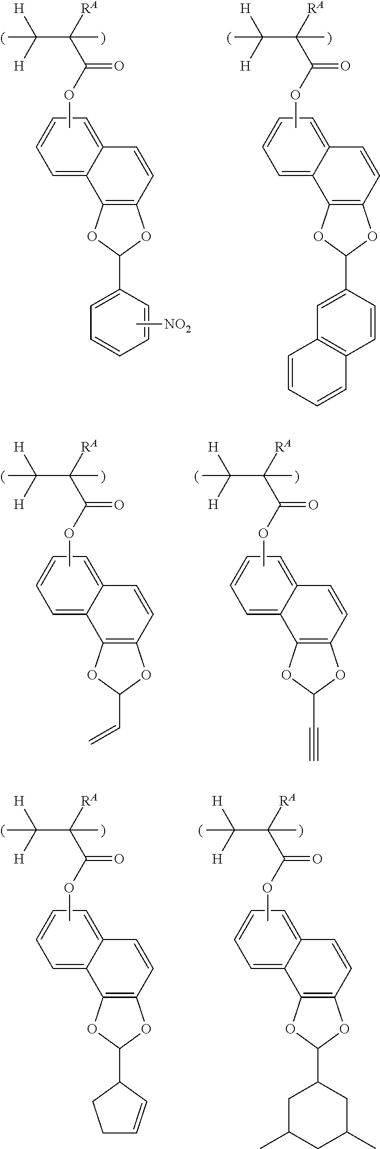

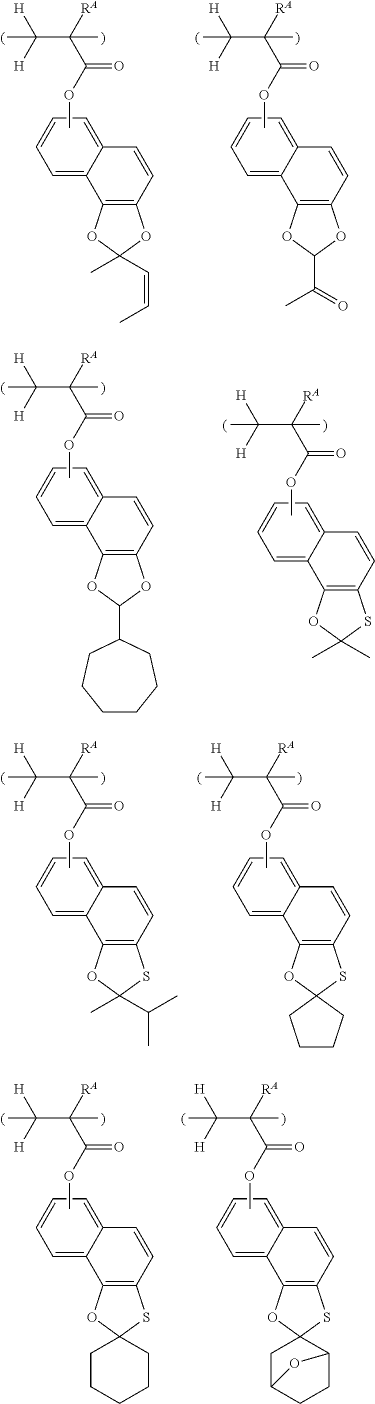

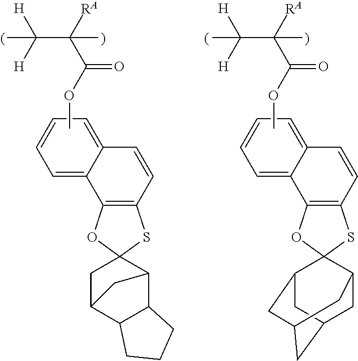

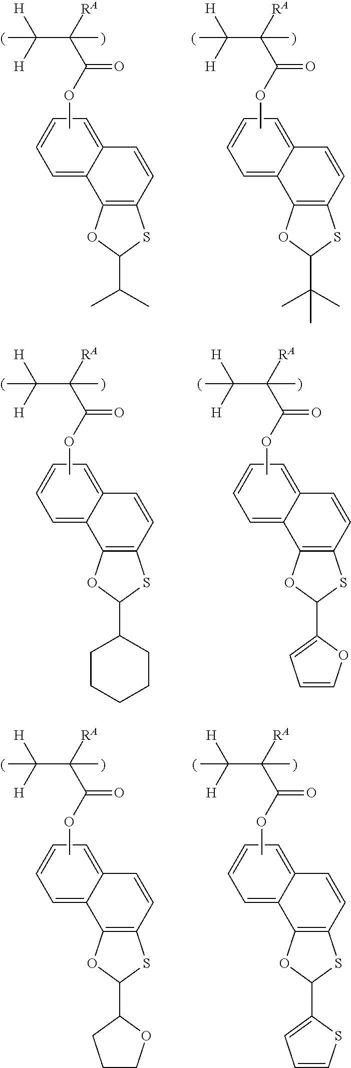

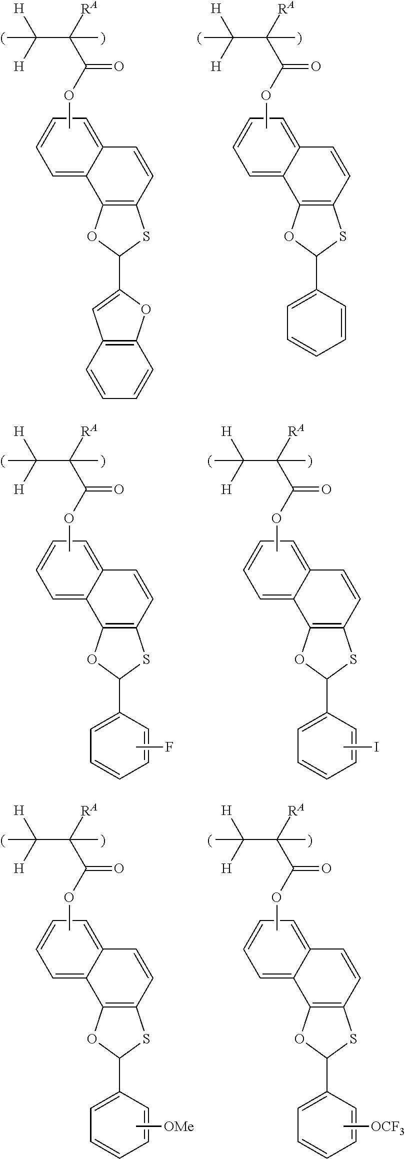

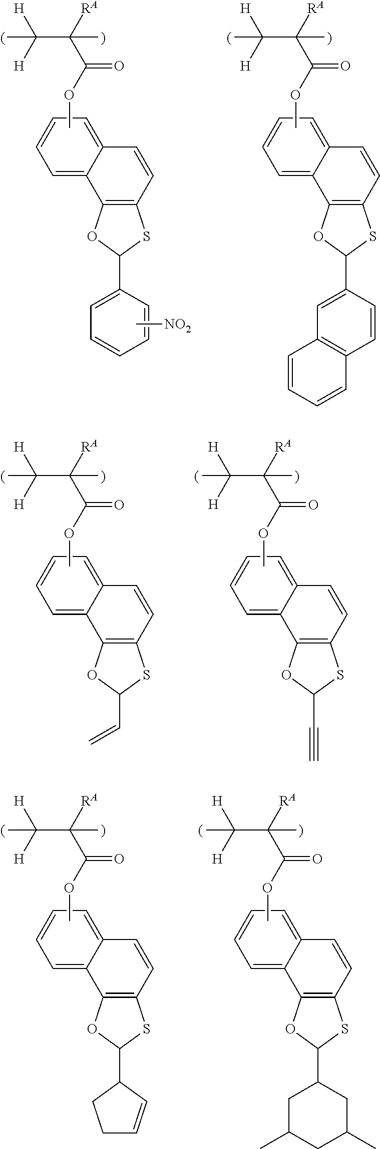

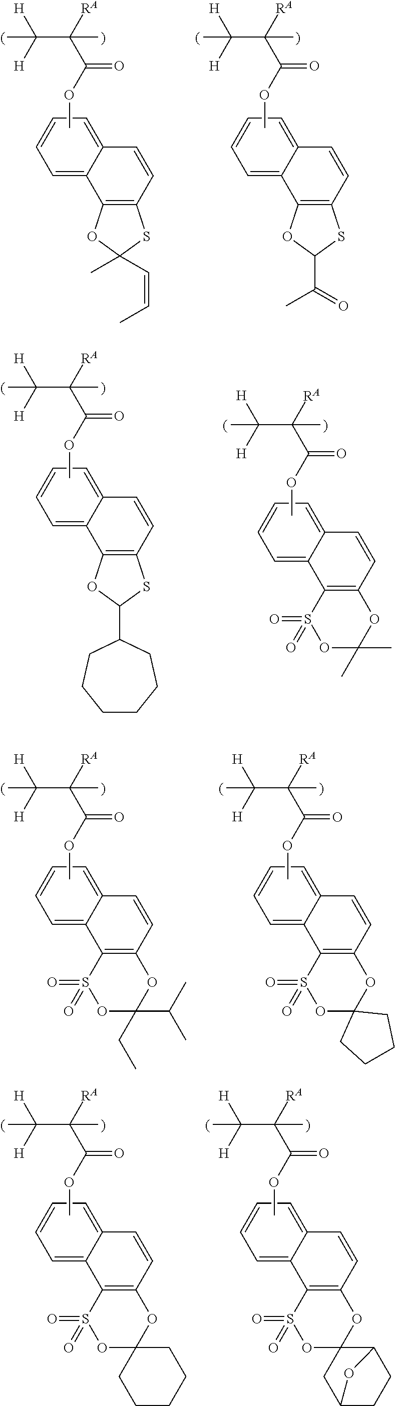

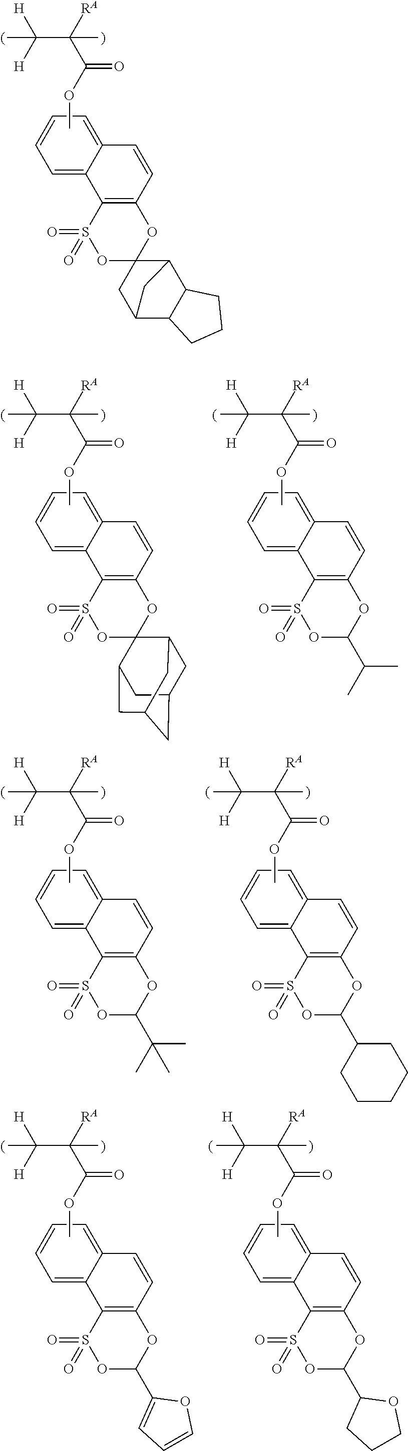

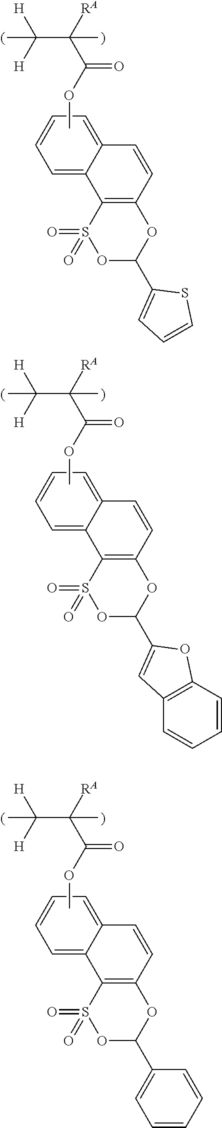

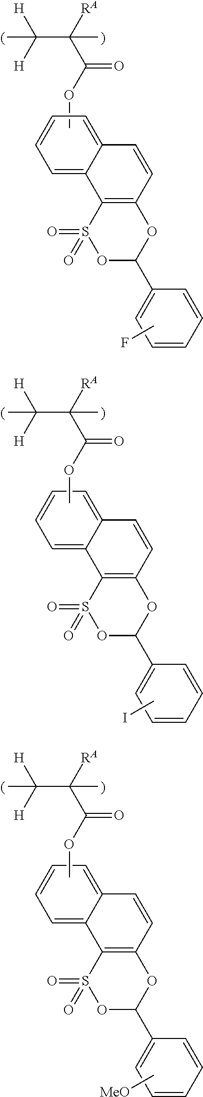

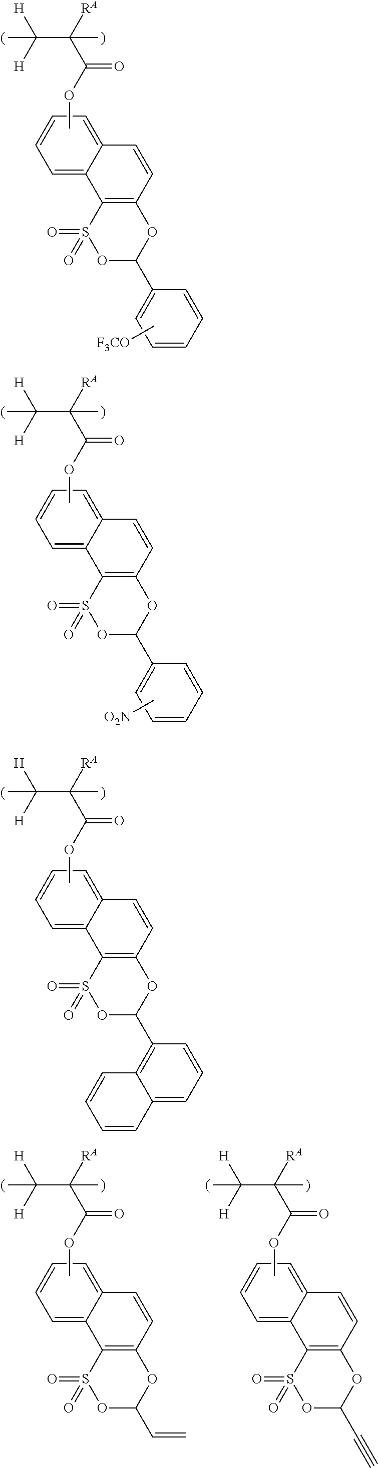

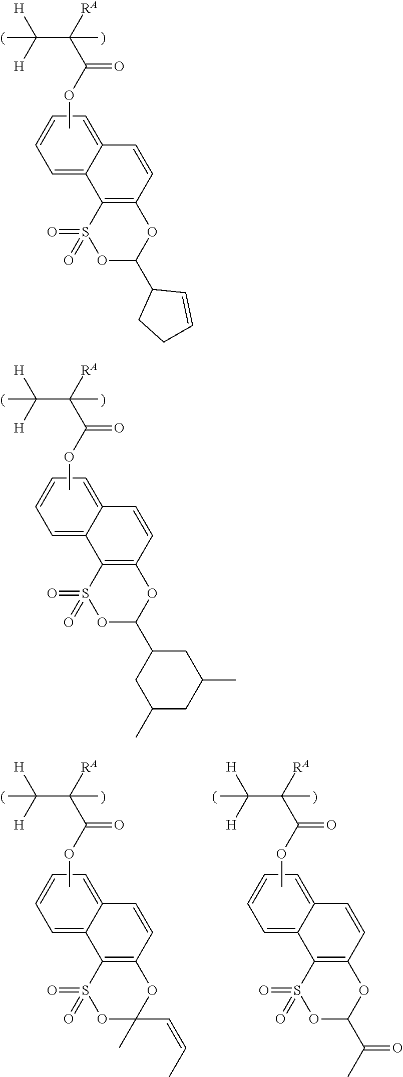

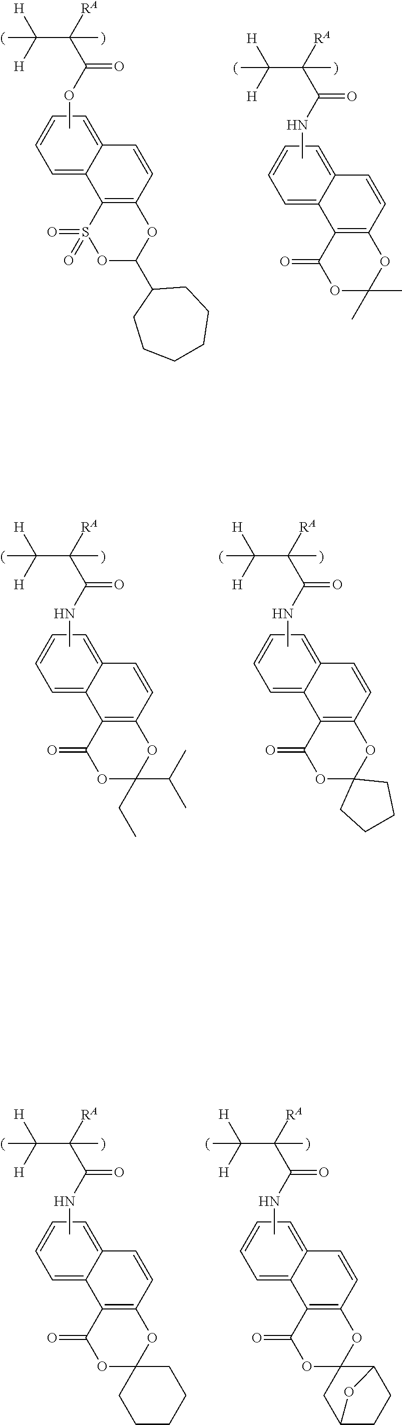

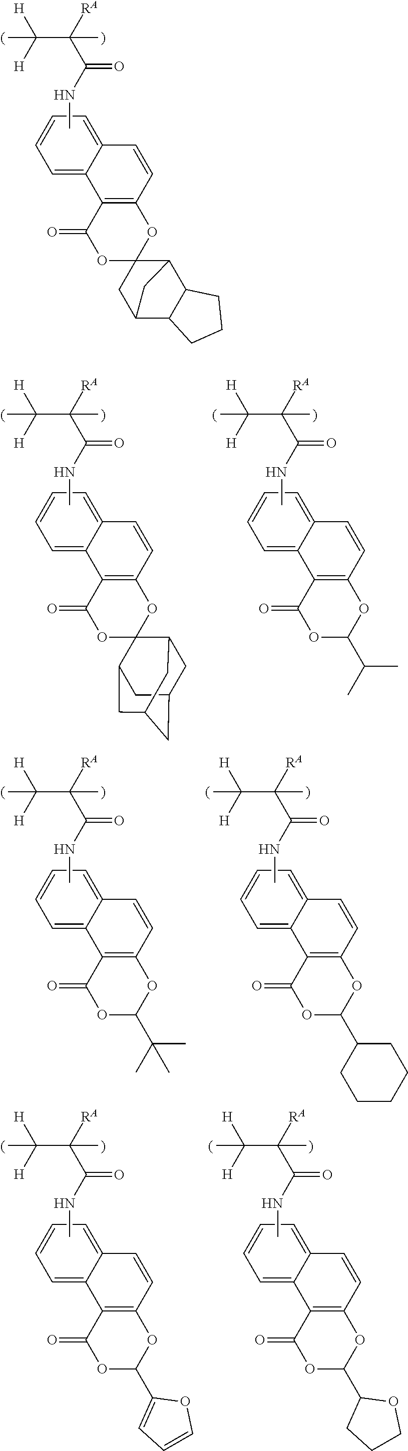

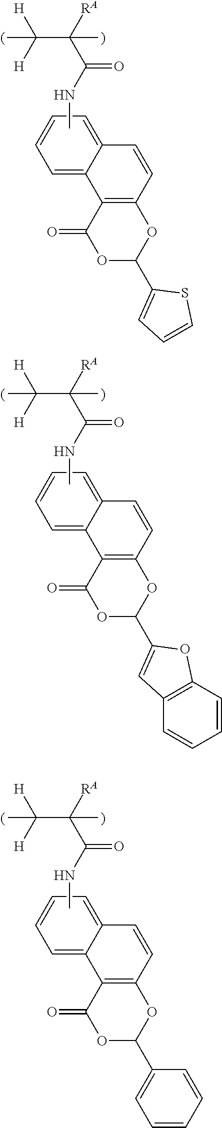

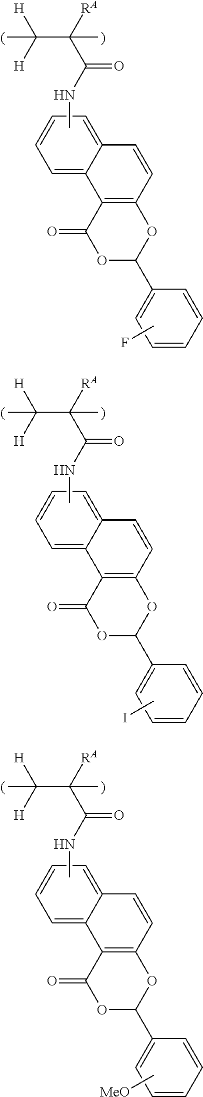

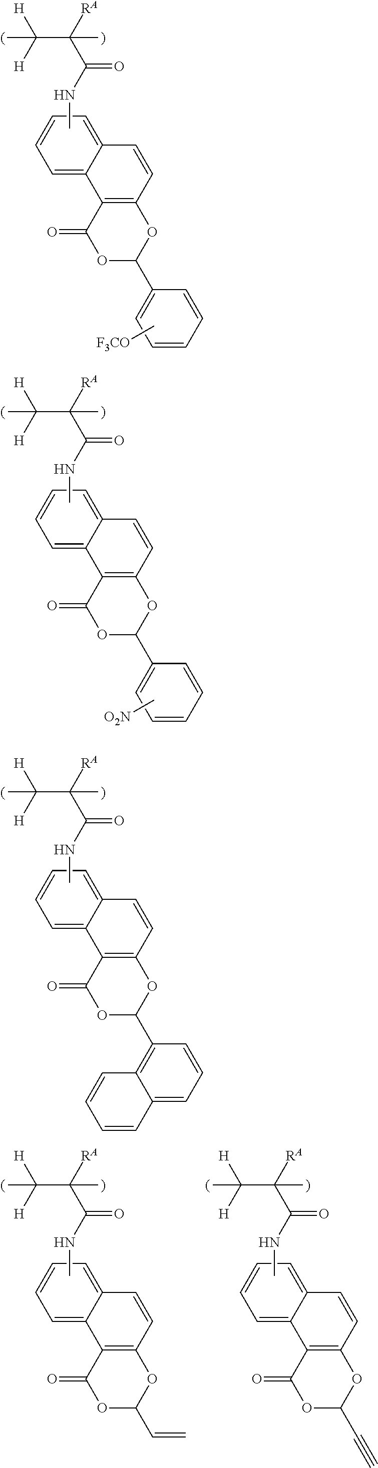

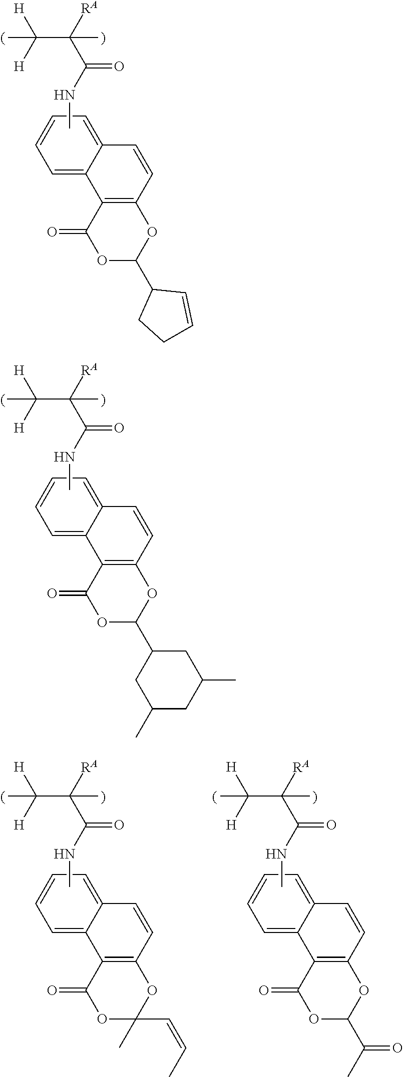

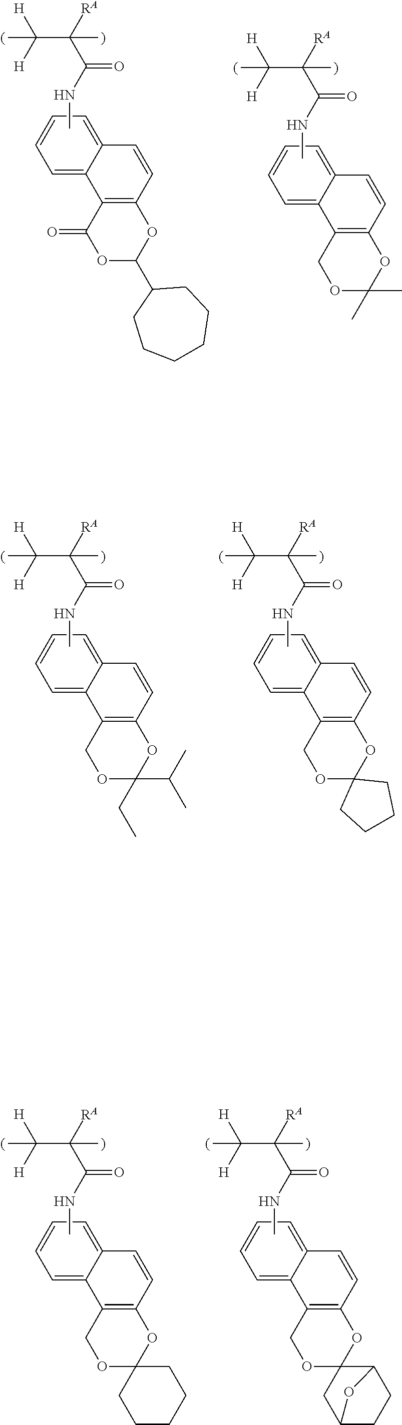

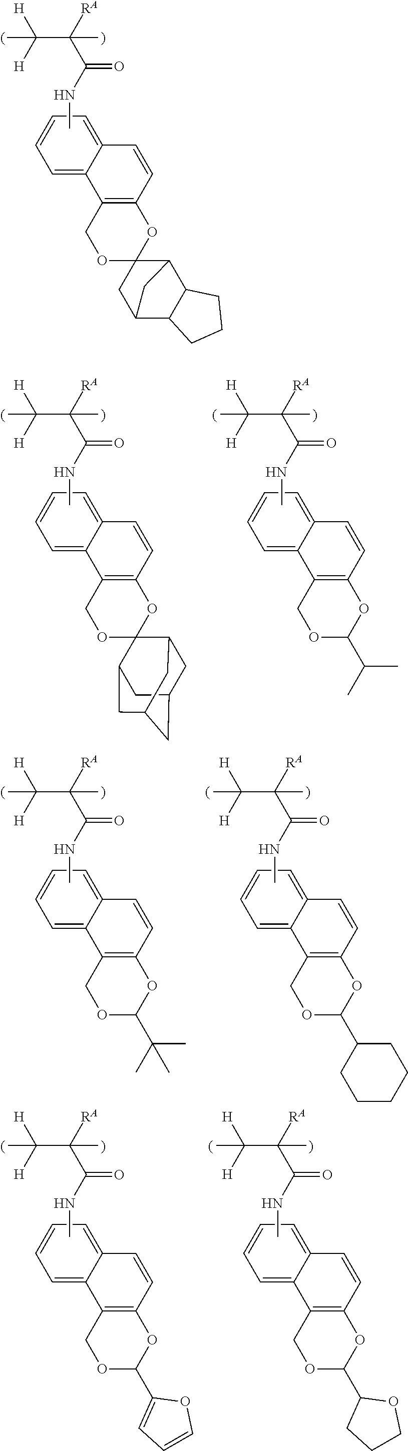

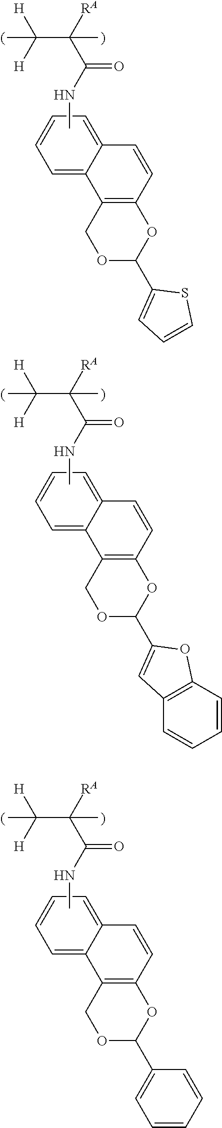

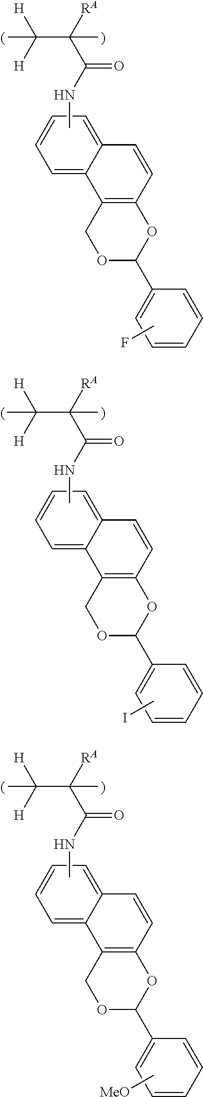

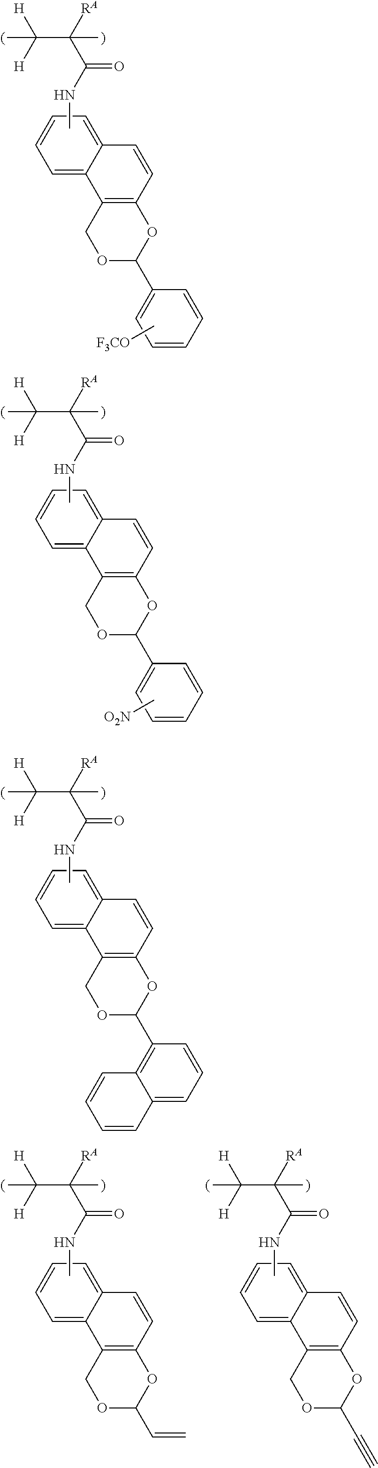

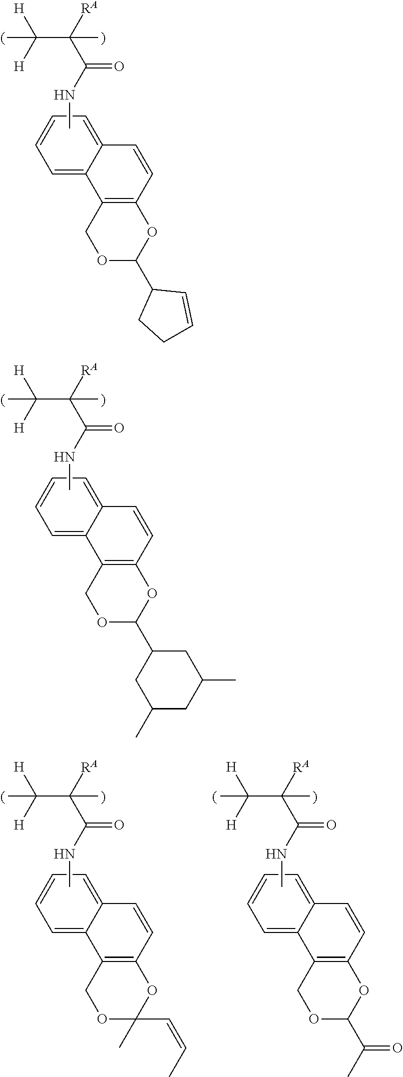

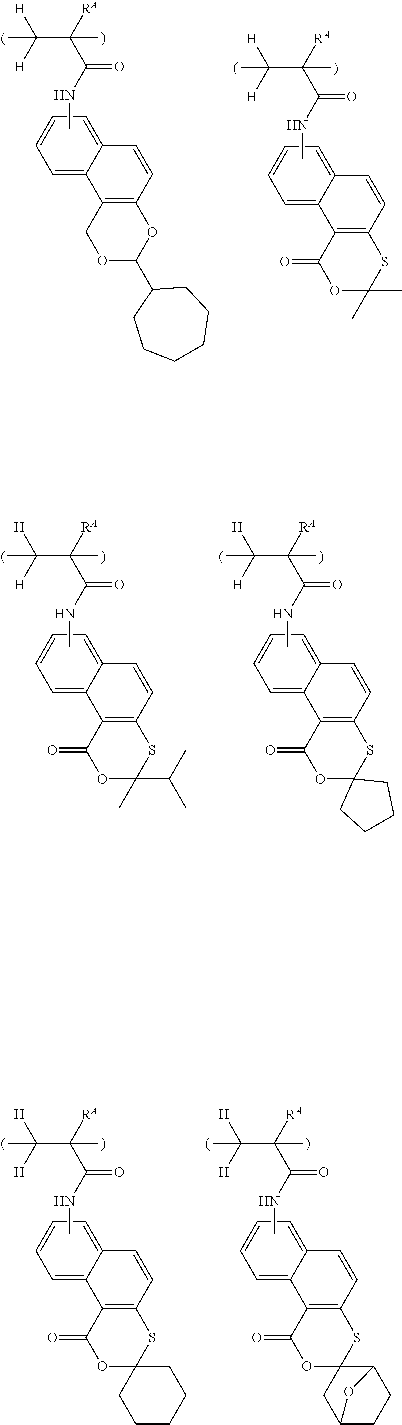

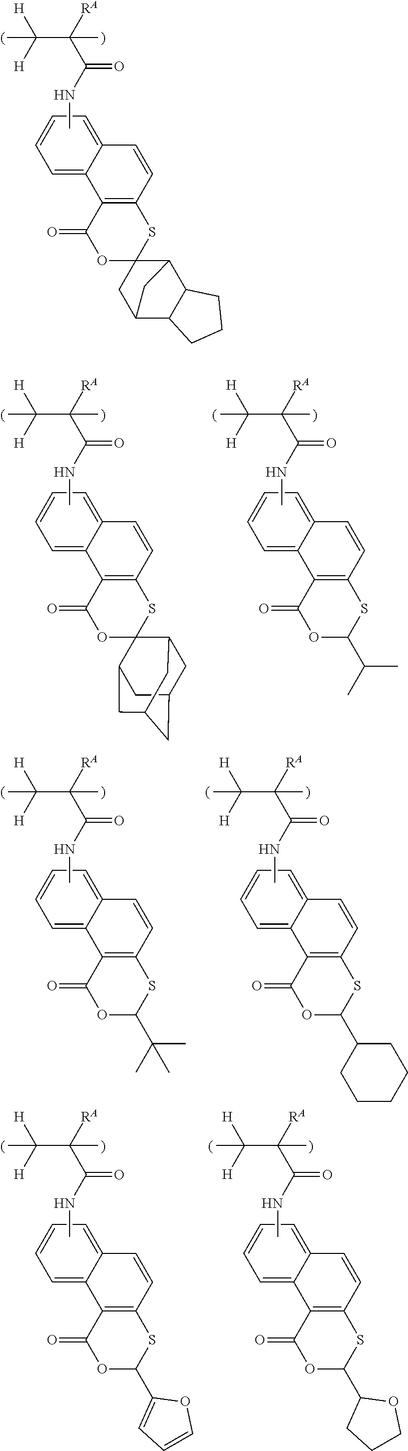

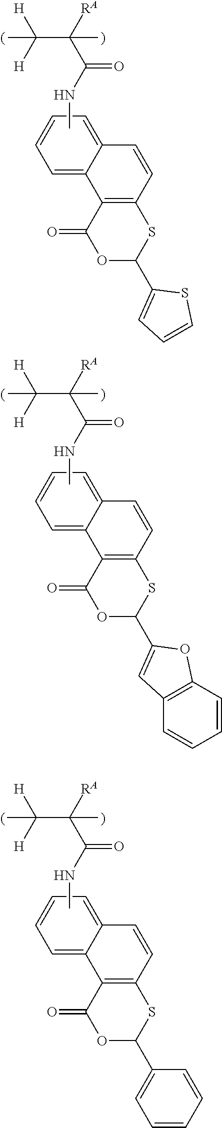

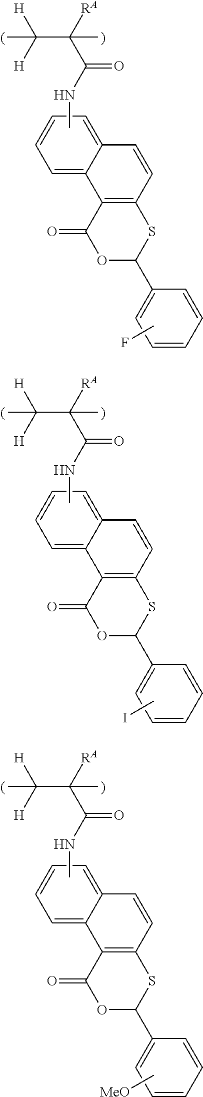

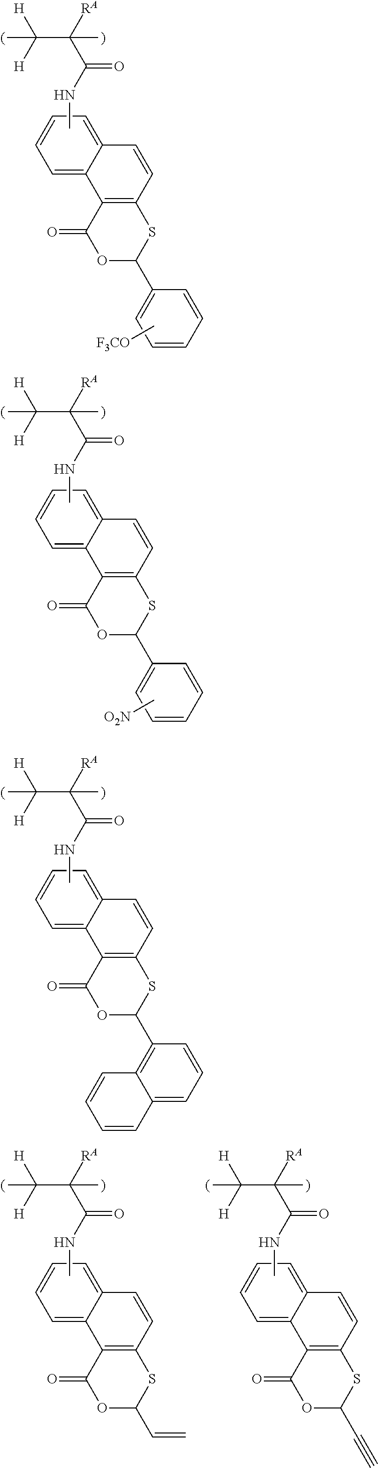

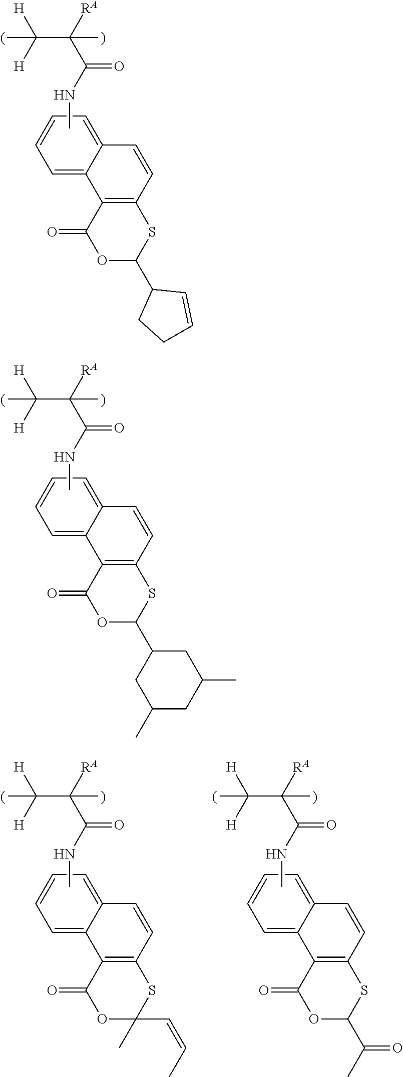

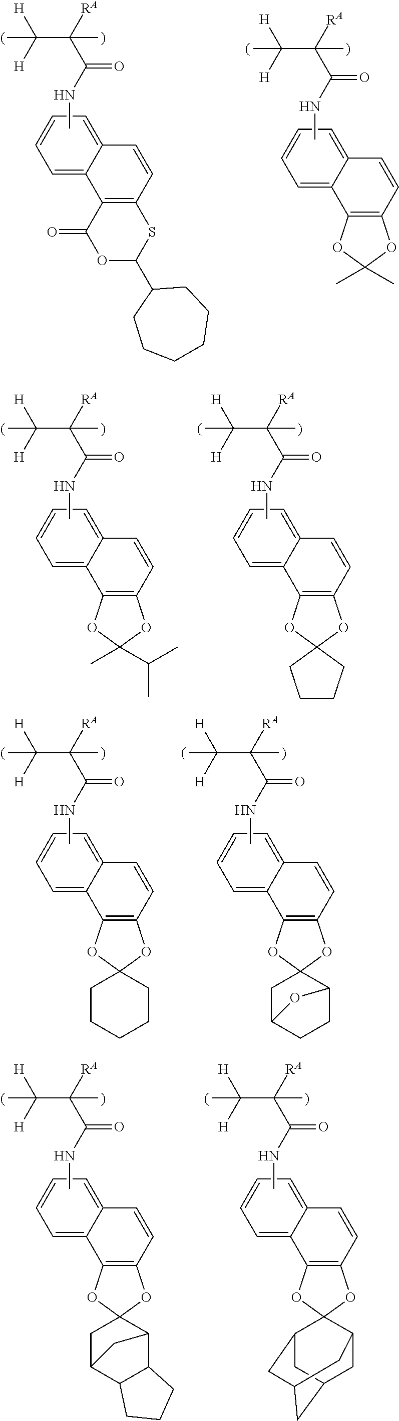

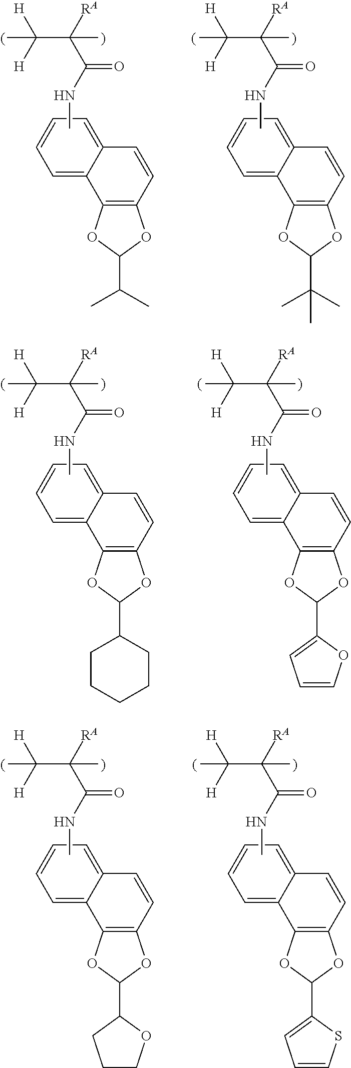

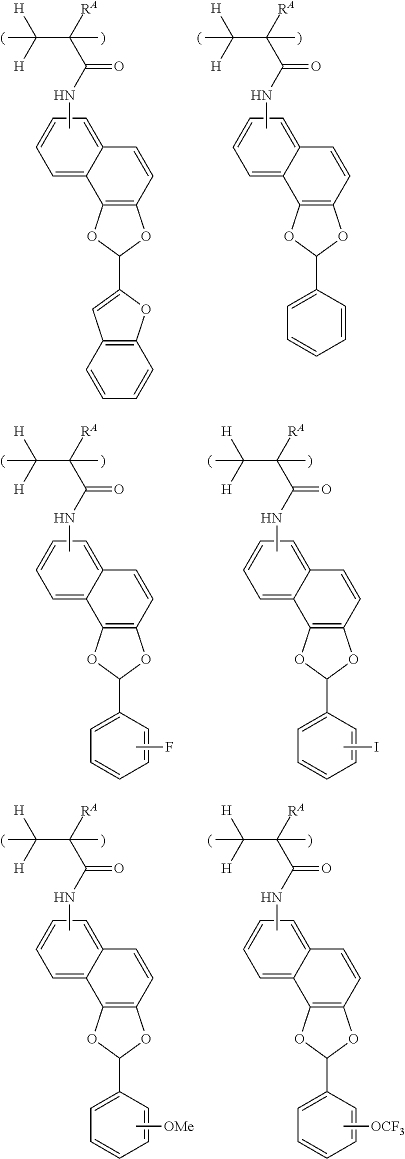

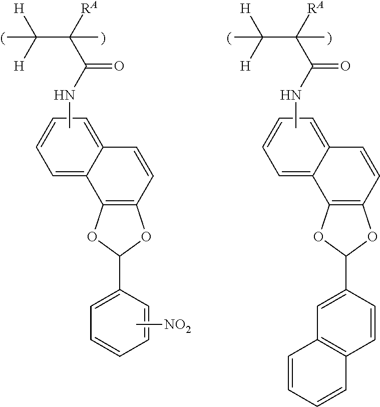

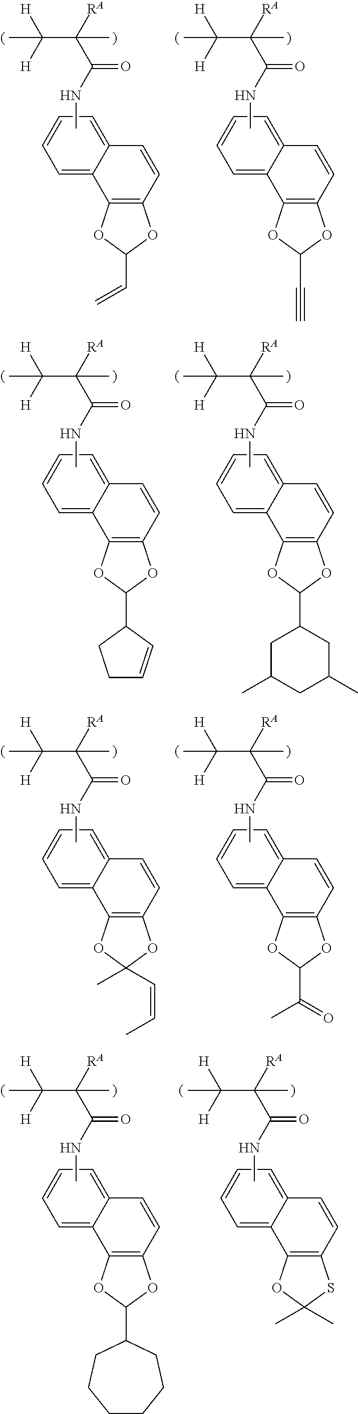

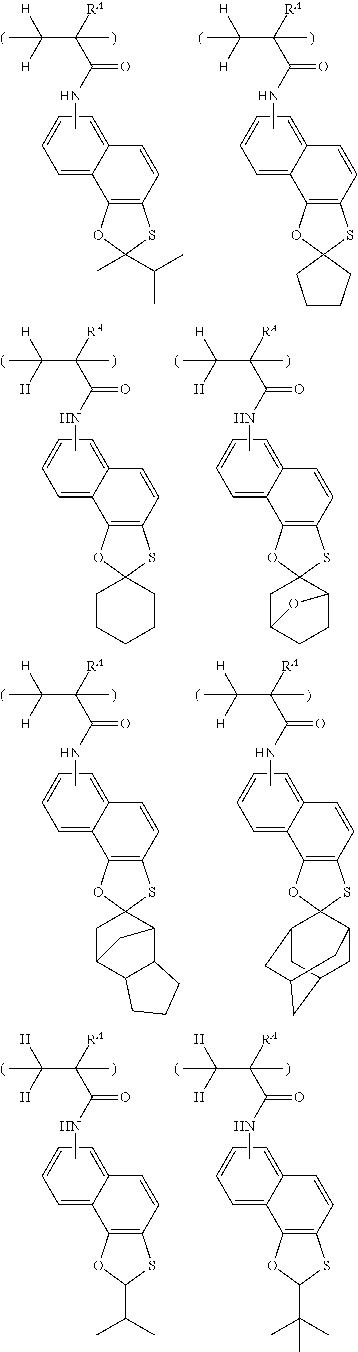

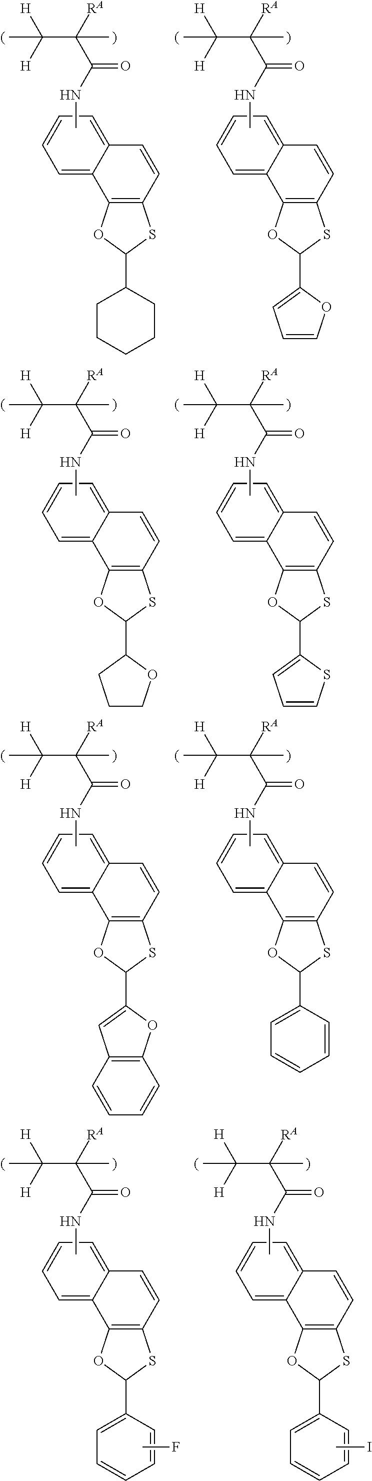

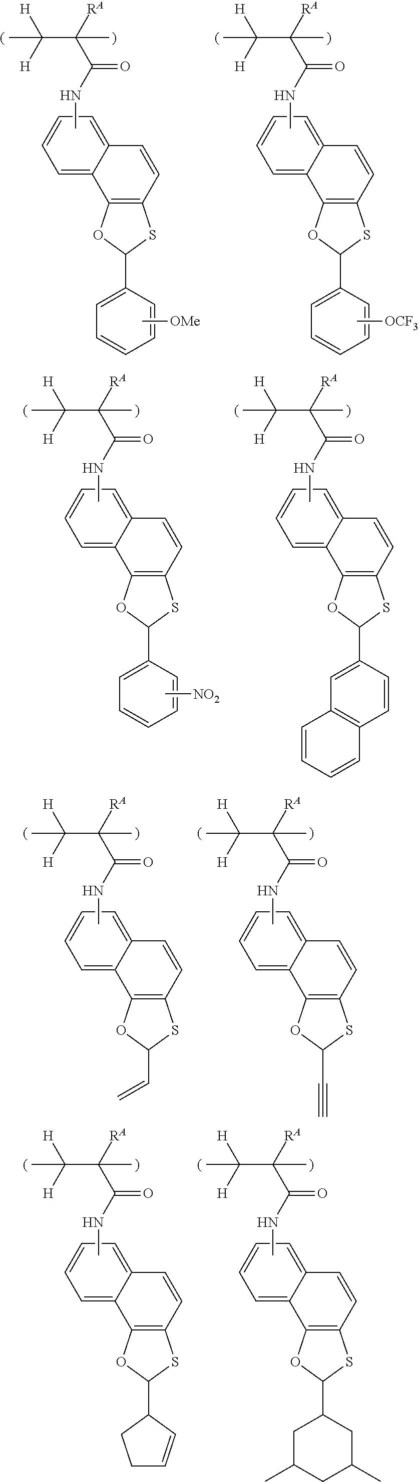

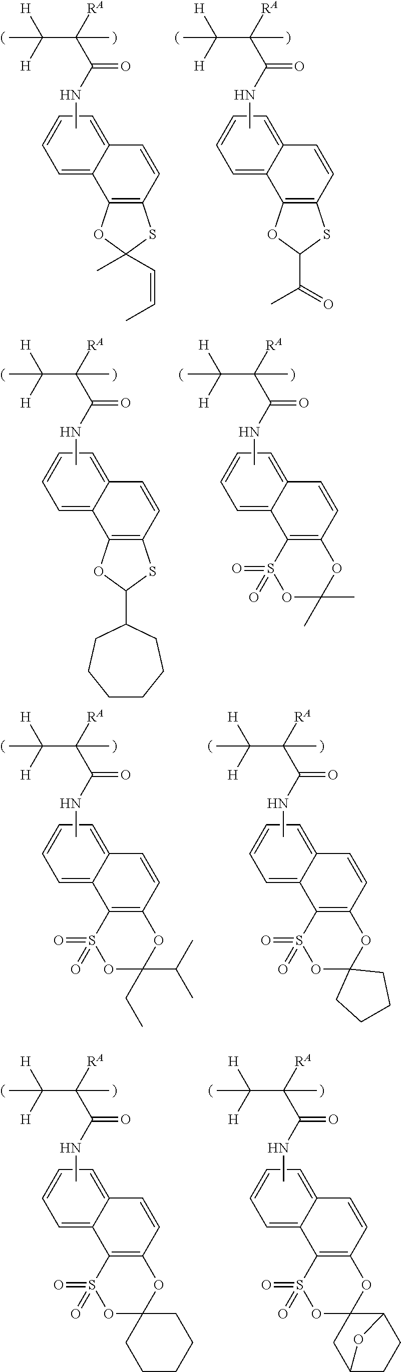

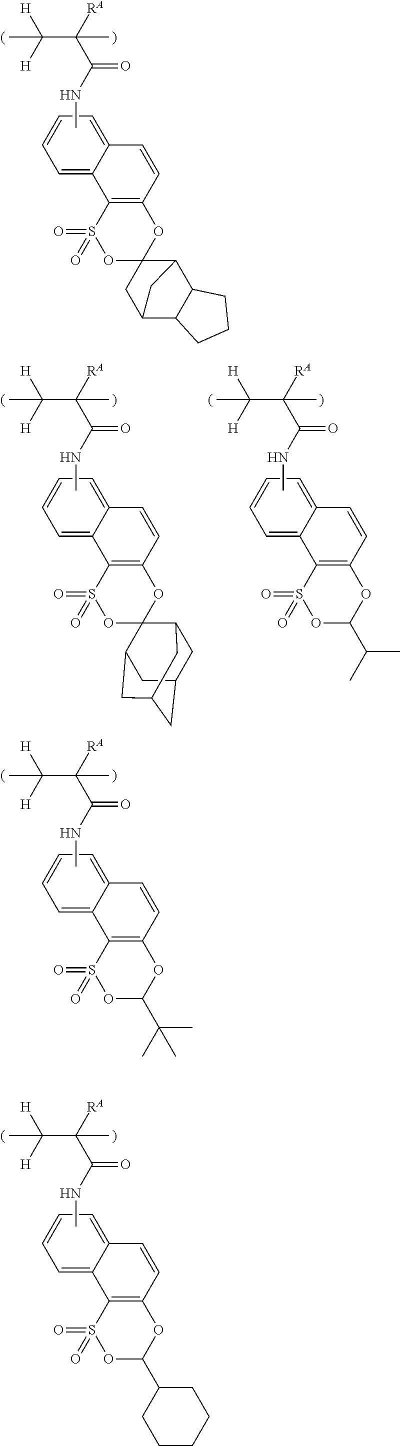

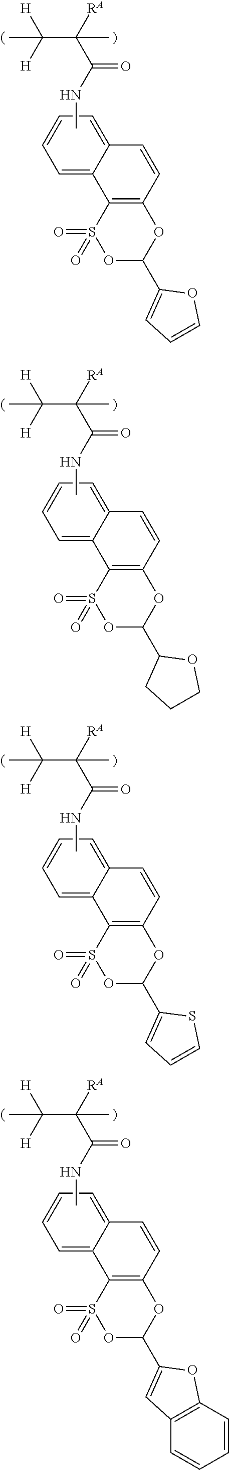

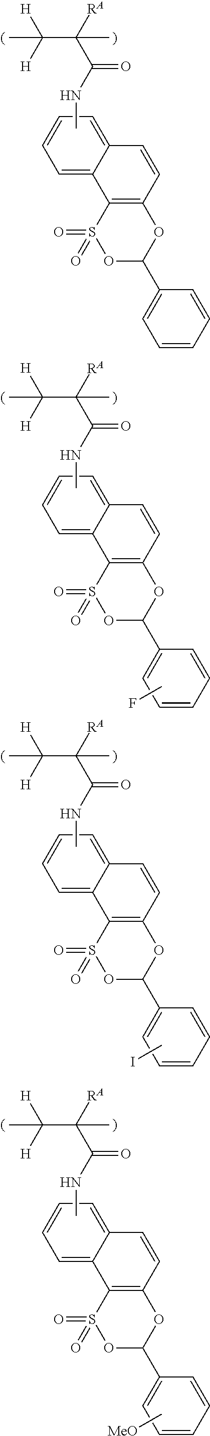

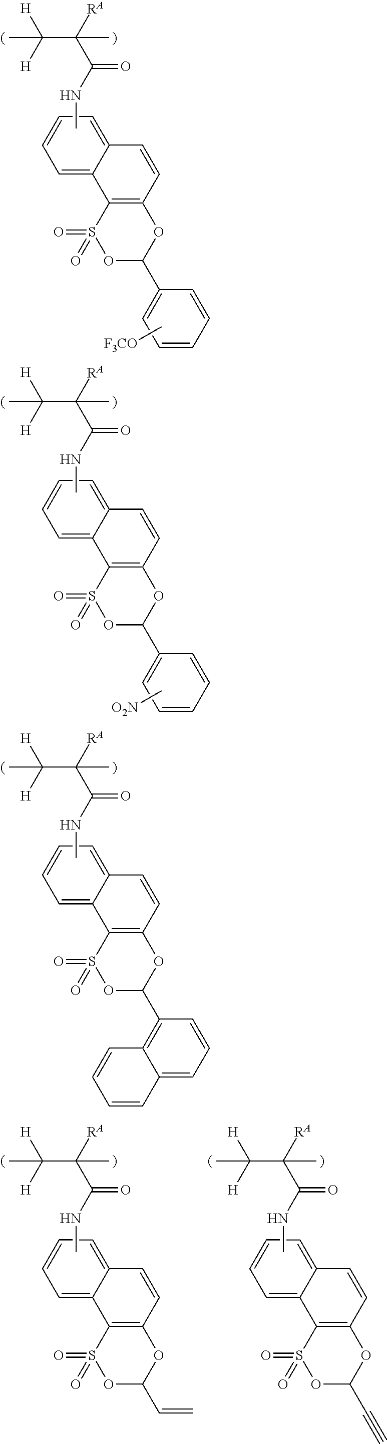

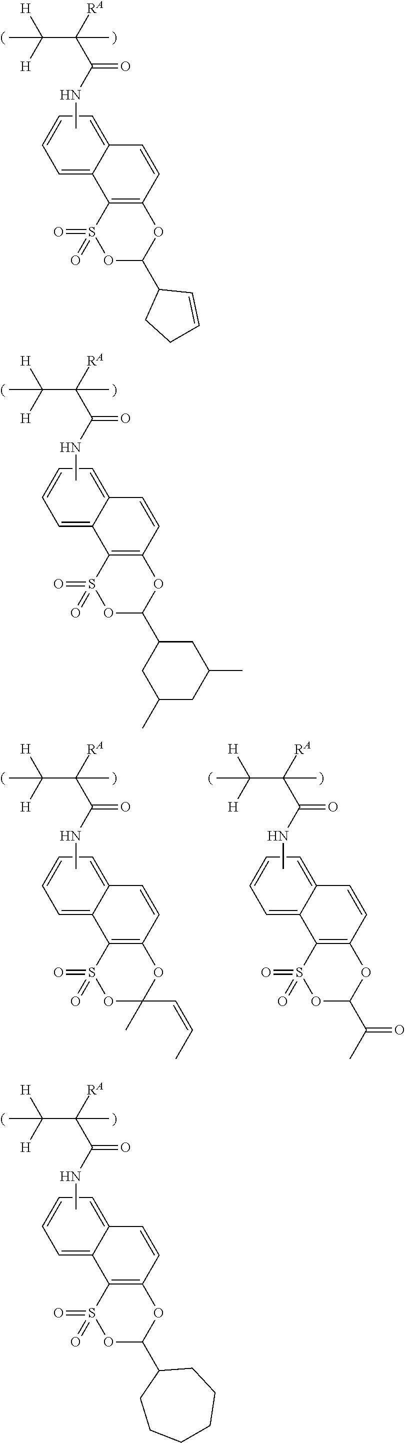

The base polymer (B) further comprises at least one constituent selected from a repeating unit represented by the following general formula (b1)(hereinafter also referred to as repeating unit-b1) and a repeating unit represented by the following general formula (b2) (hereinafter also referred to as repeating unit-b2).

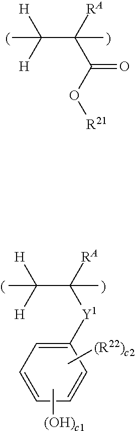

In the formulae, “c1” is an integer from 1 to 4; “c2” is an integer from 0 to 4; and 1≤c1+c2≤5;
R^{A} is each independently a hydrogen atom, a fluorine atom, a methyl group, or a trifluoromethyl group;
Y¹ is a single bond or *—C(═O)—O—; “*” represents a bond to a main chain carbon atom;
R²¹ is a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R²² is a halogen atom, a hydroxy group, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” is 2, 3, or 4, each R²² may be the same as or different from each other.

In the general formulae (bl) and (b2), R^{A} is each independently a hydrogen atom, a fluorine atom, a methyl group, or a trifluoromethyl group. Y¹ is a single bond or *—C(═O)—O—. “*” represents a bond to a main chain carbon atom. R²¹ is a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—). R²² is a halogen atom, a hydroxy group, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “c1” is an integer from 1 to 4. “c2” is an integer from 0 to 4. Note, 15c1+c255. When “c2” is 2, 3, or 4, each R²² may be the same as or different from each other.

Specific examples of the repeating unit-b1 include, but are not limited to, those shown below. Note that, in the following formulae, R^{A} is as defined above.

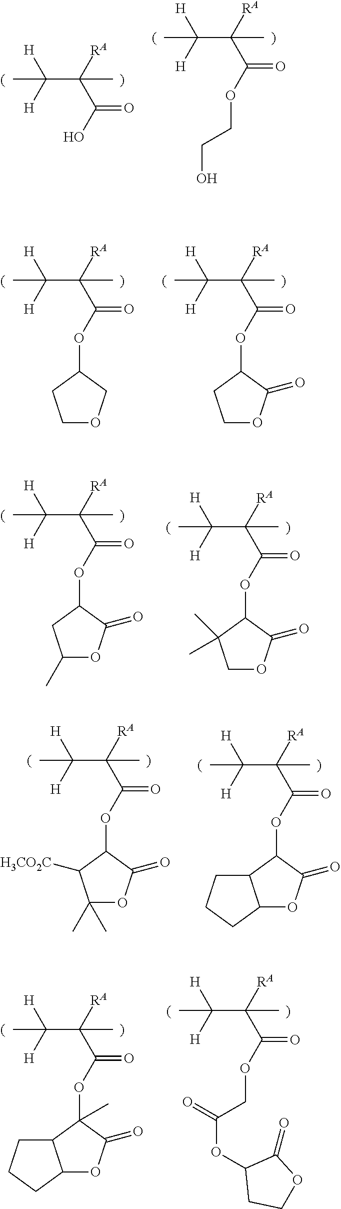

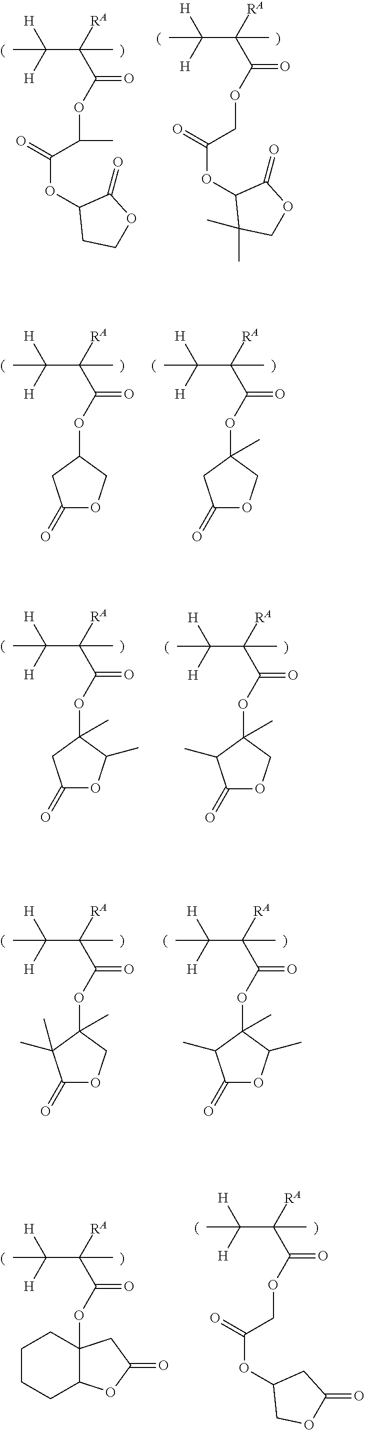

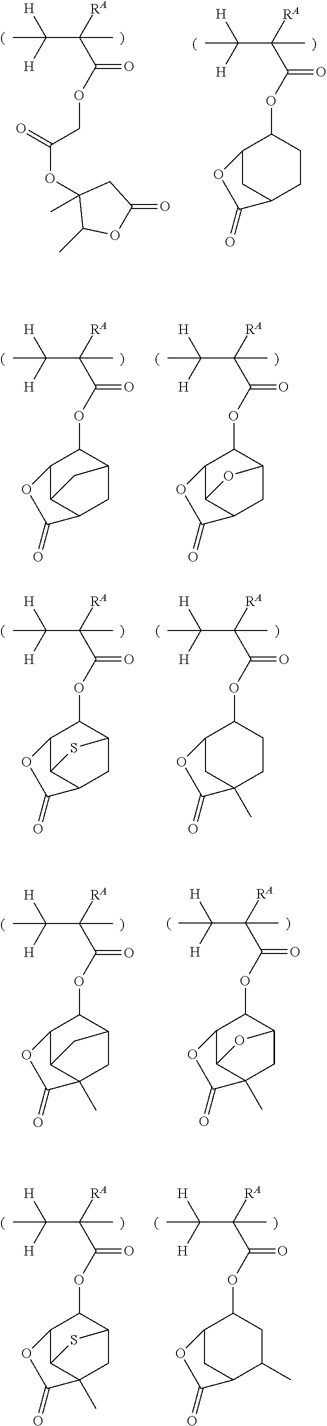

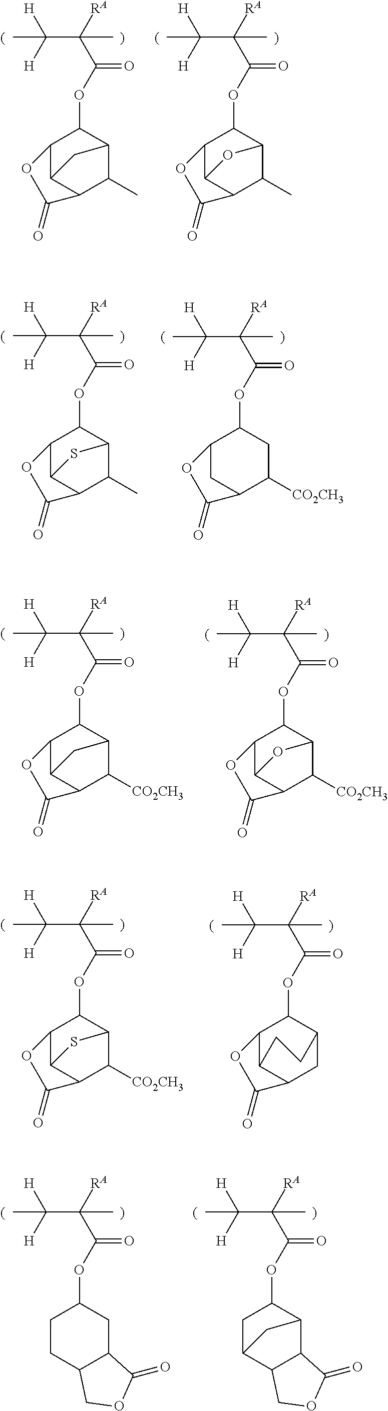

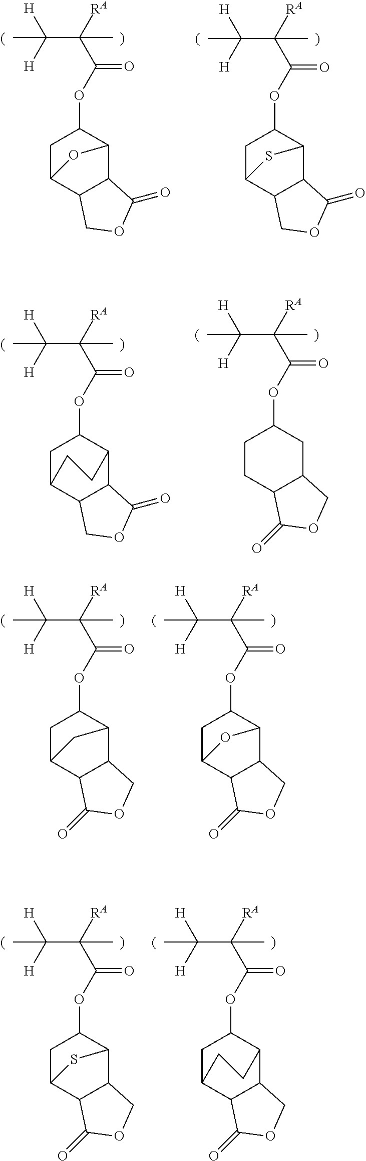

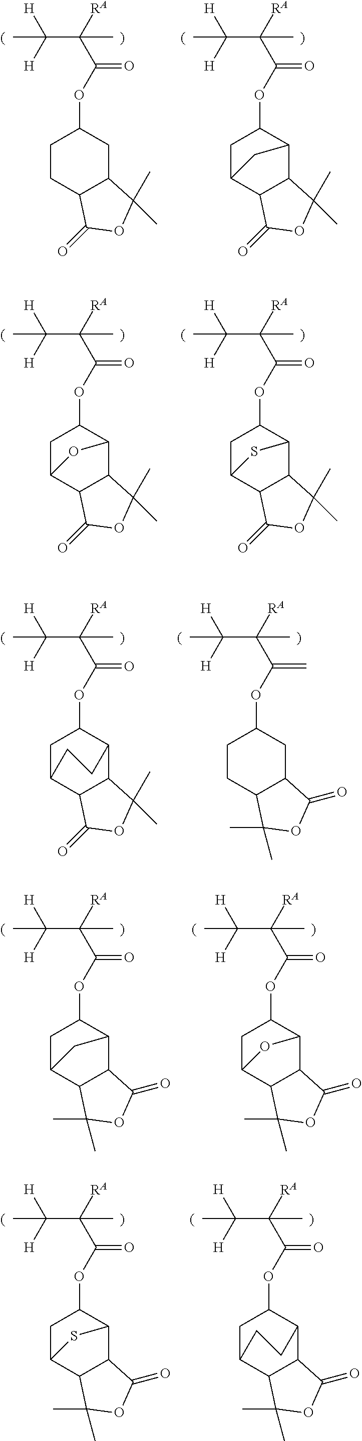

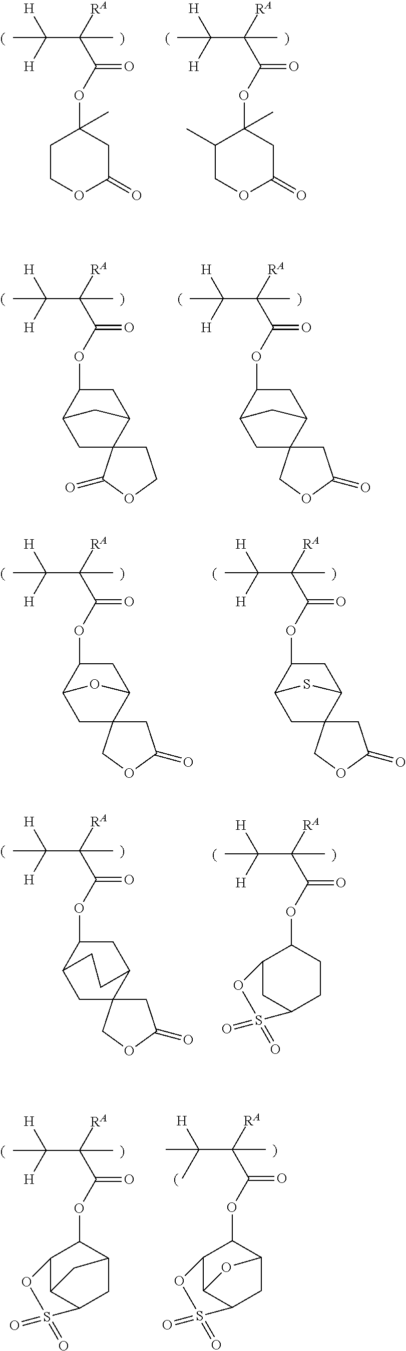

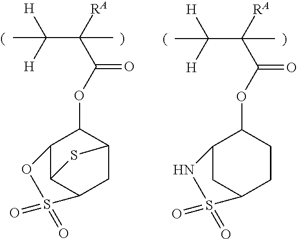

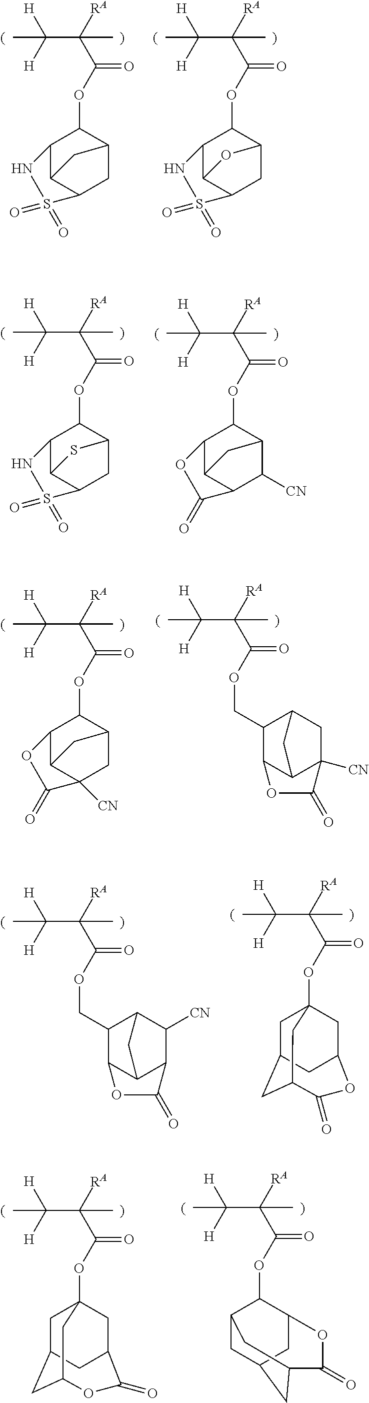

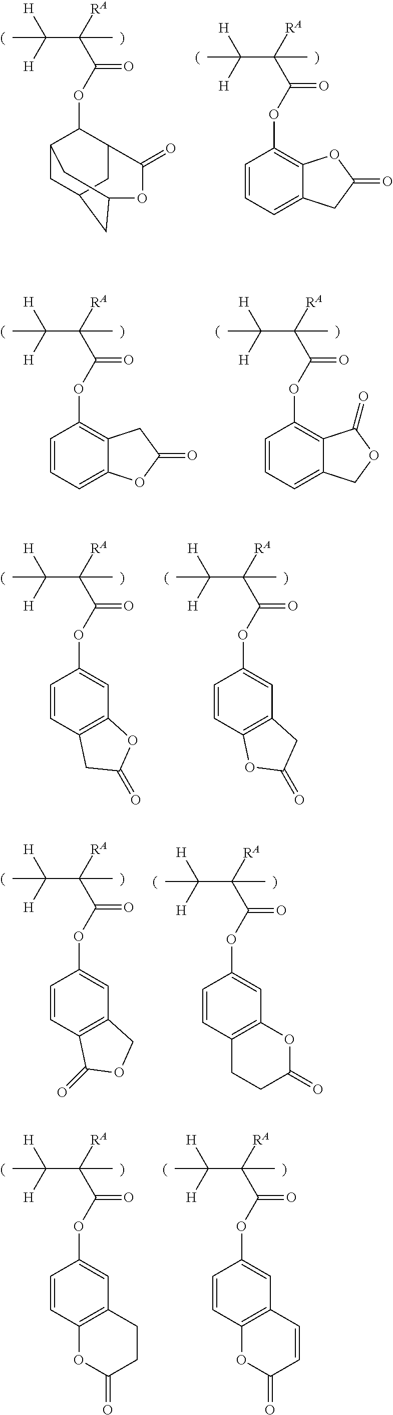

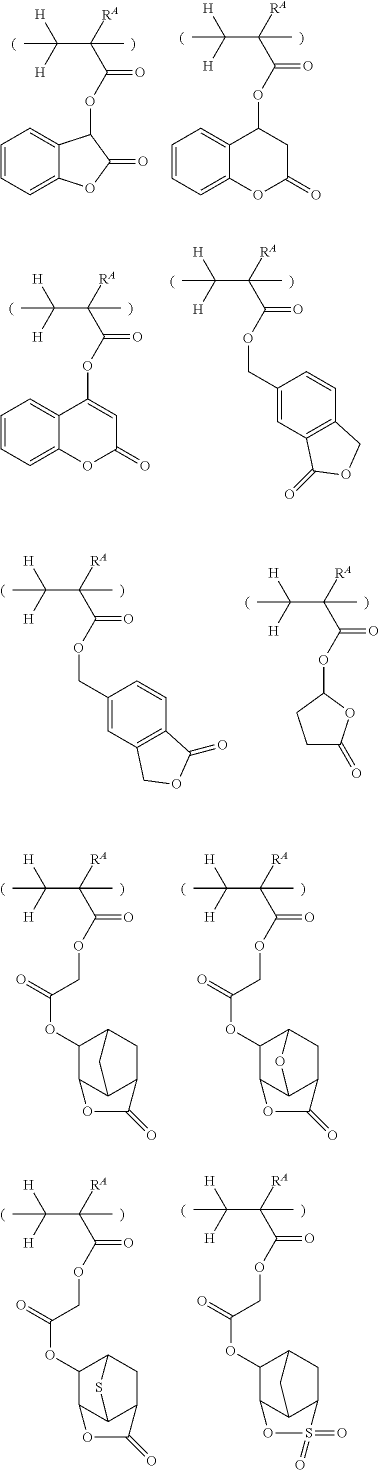

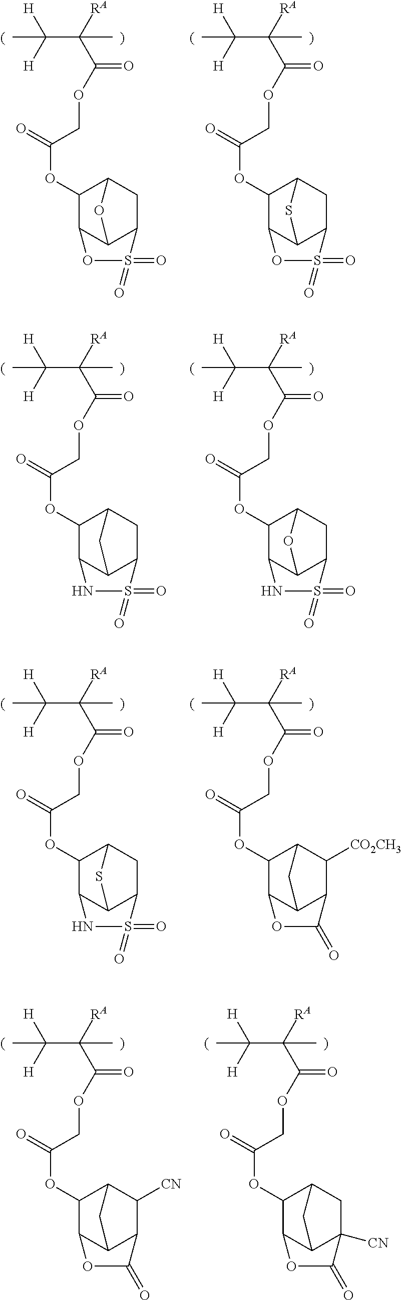

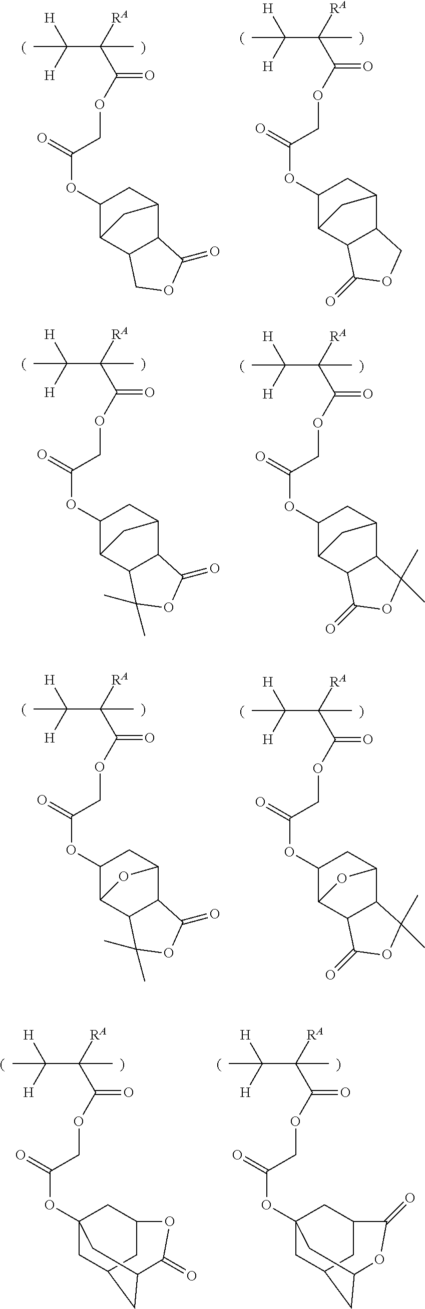

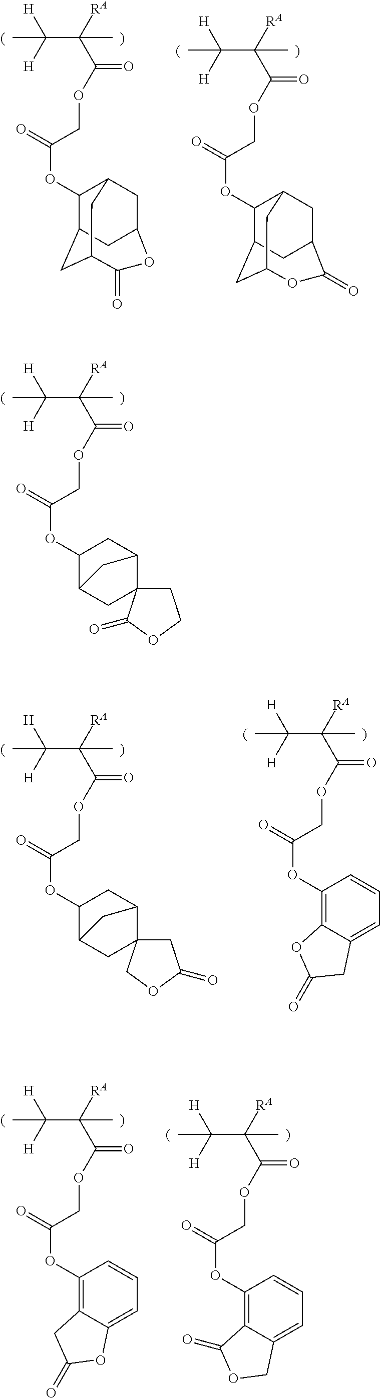

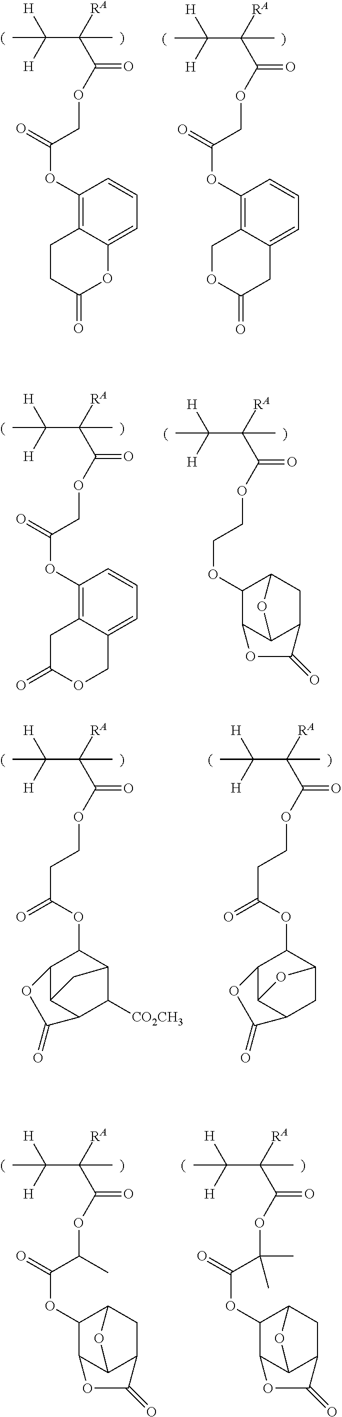

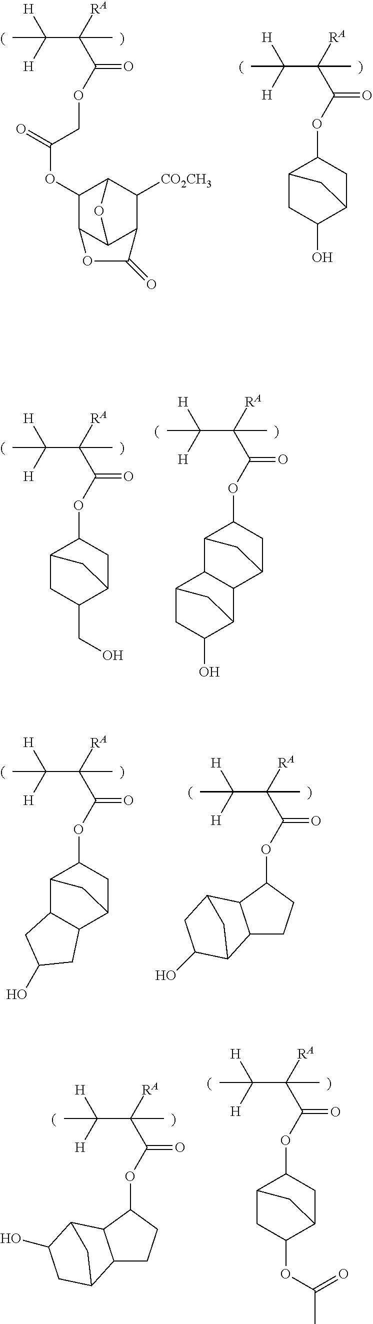

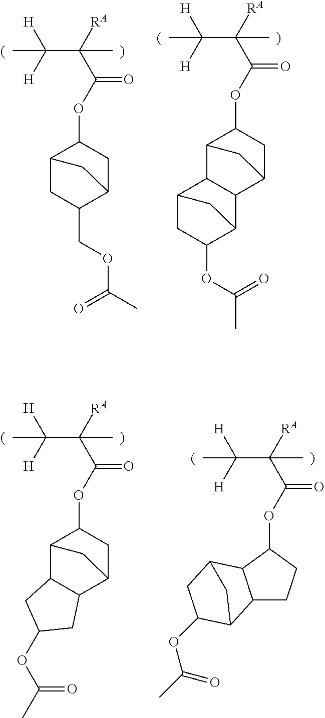

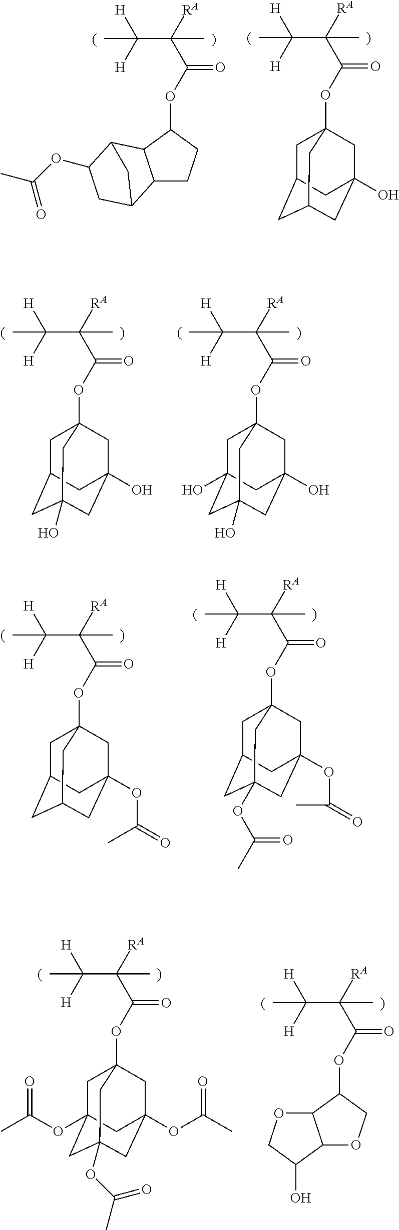

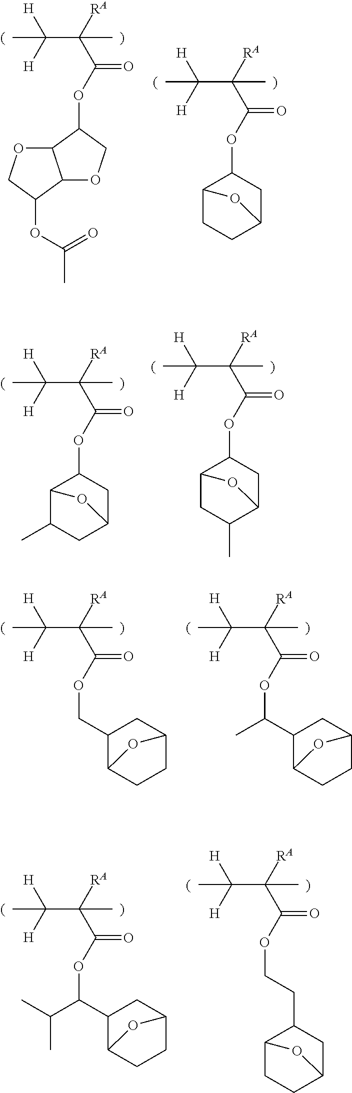

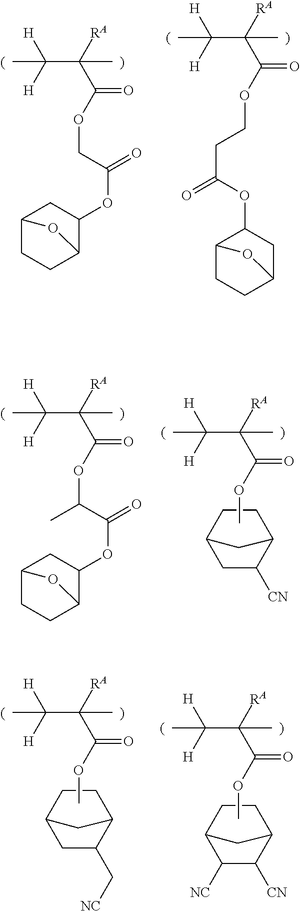

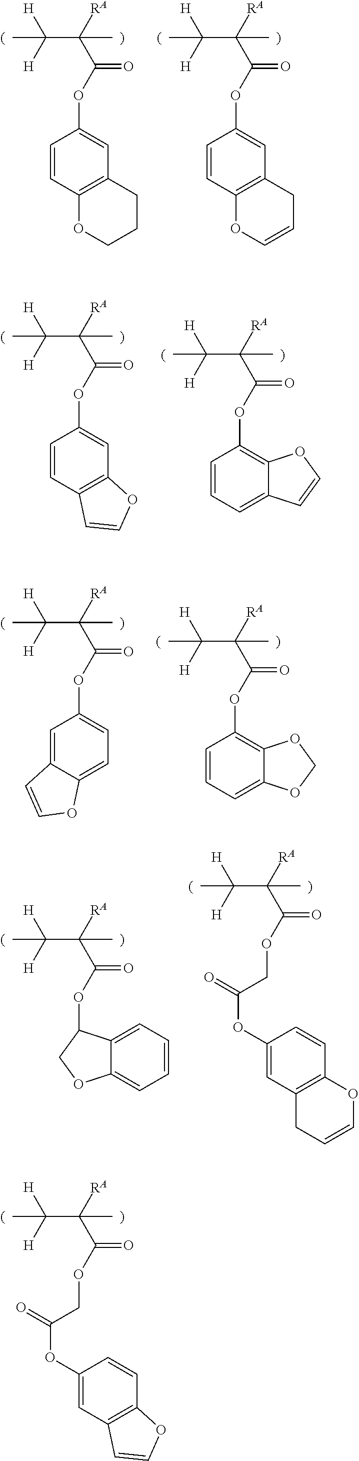

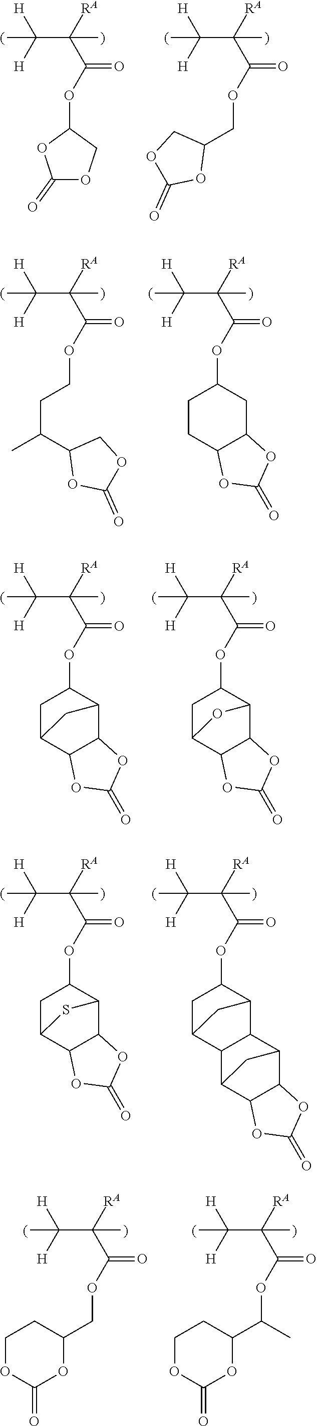

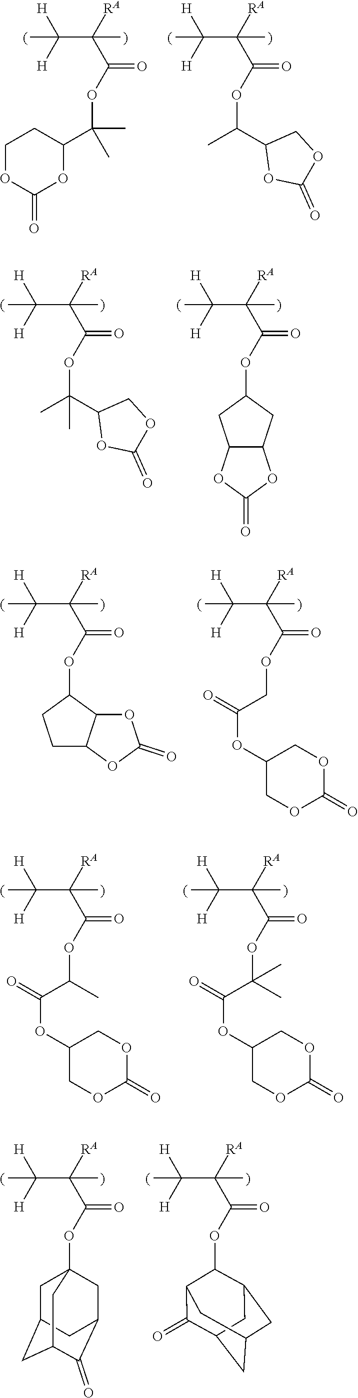

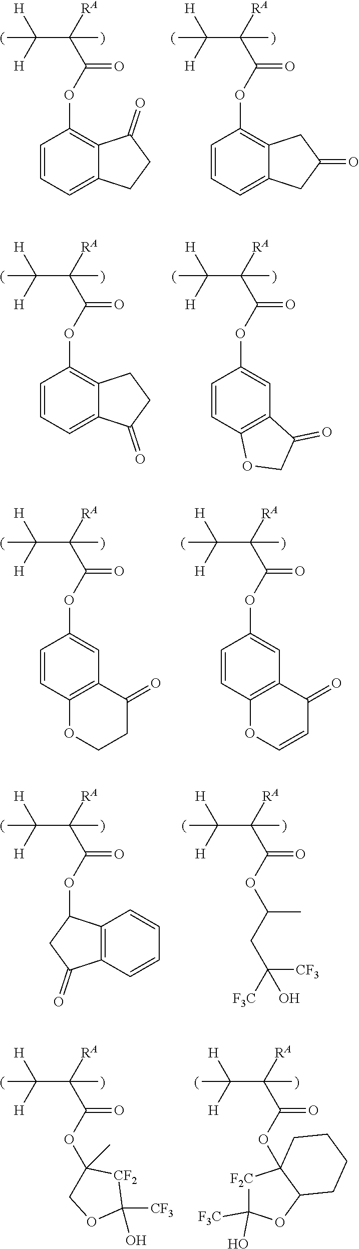

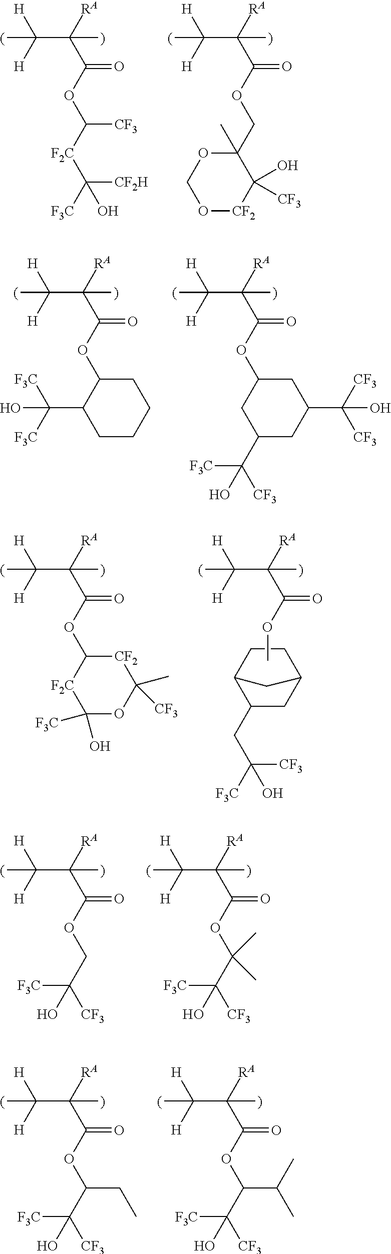

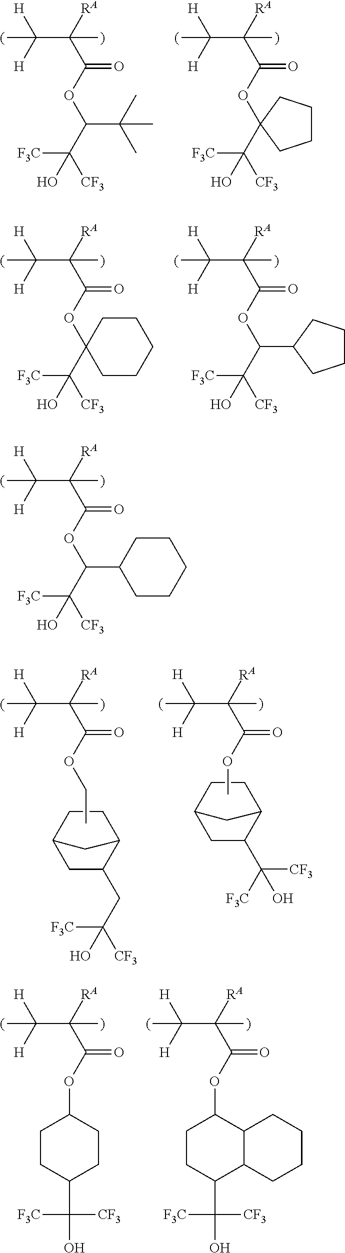

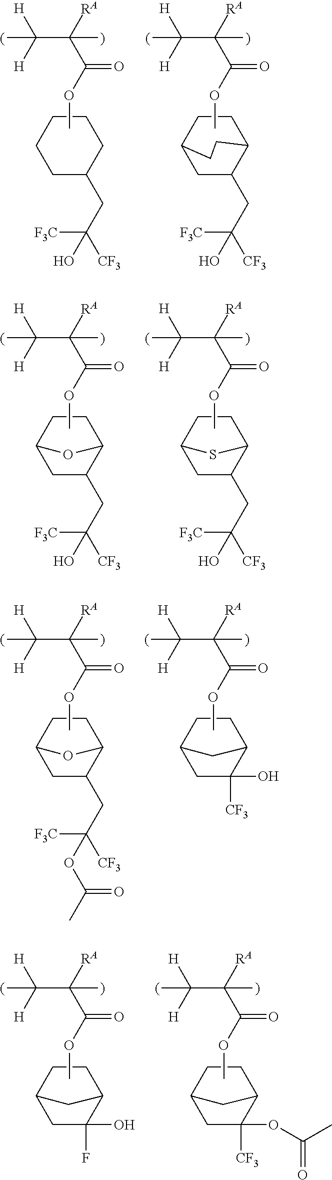

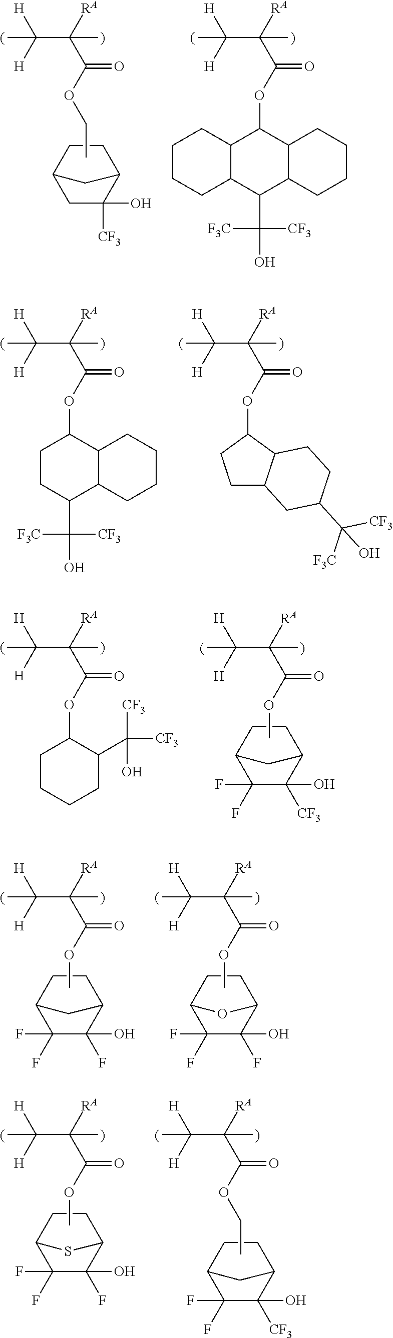

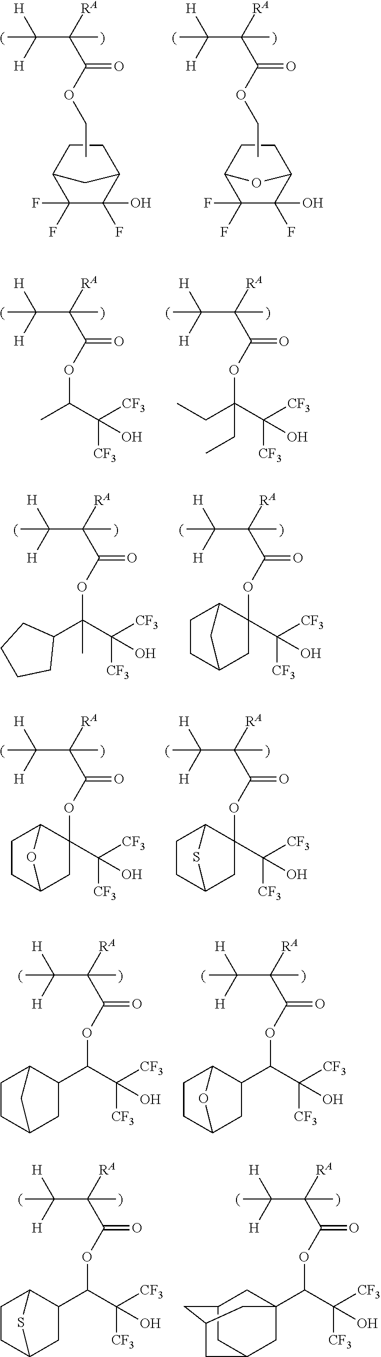

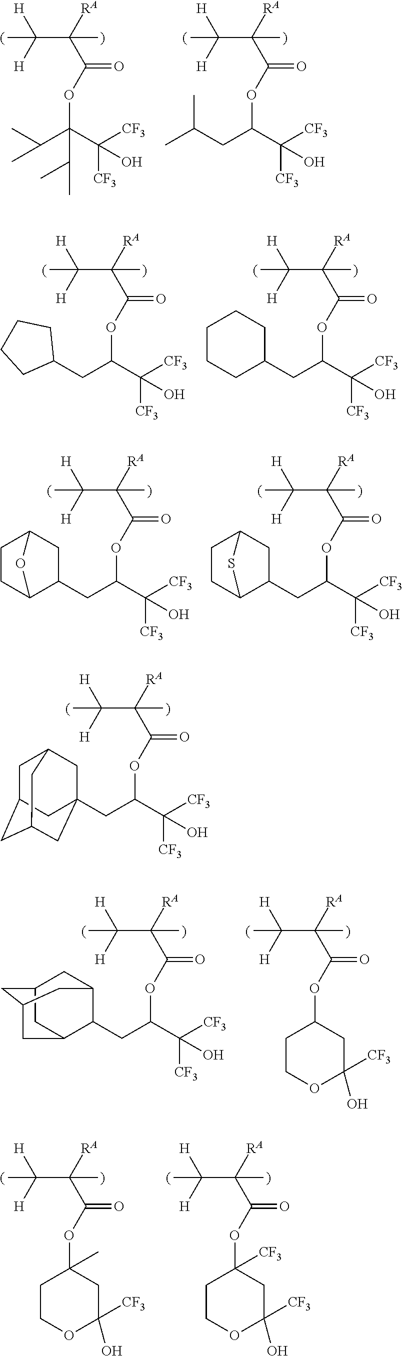

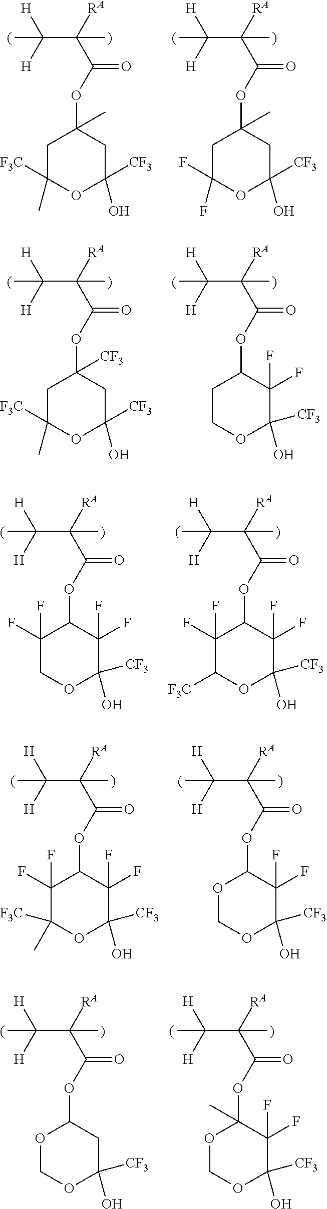

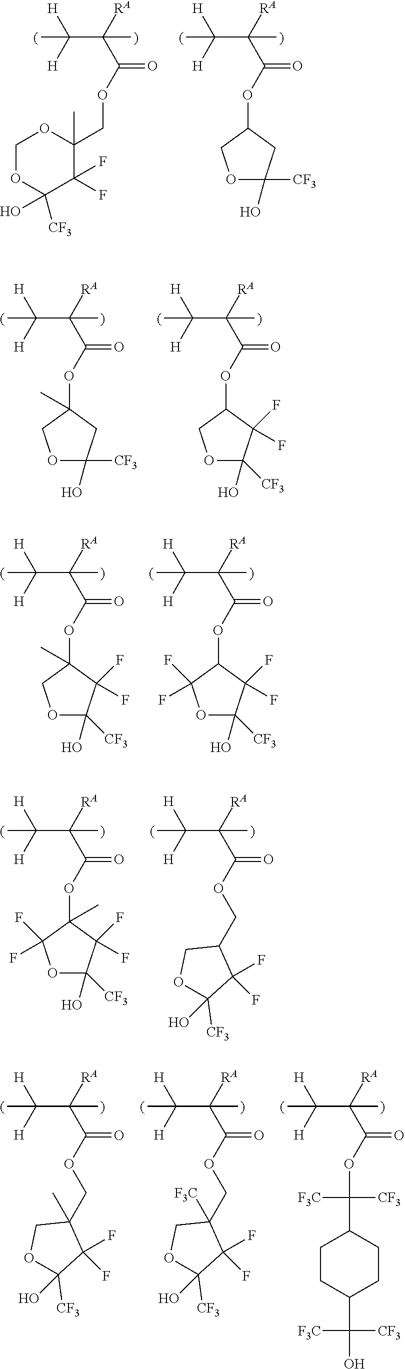

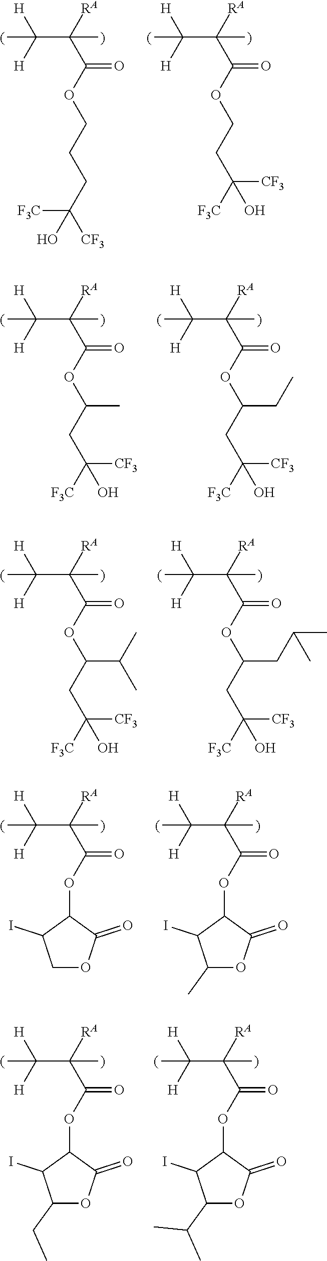

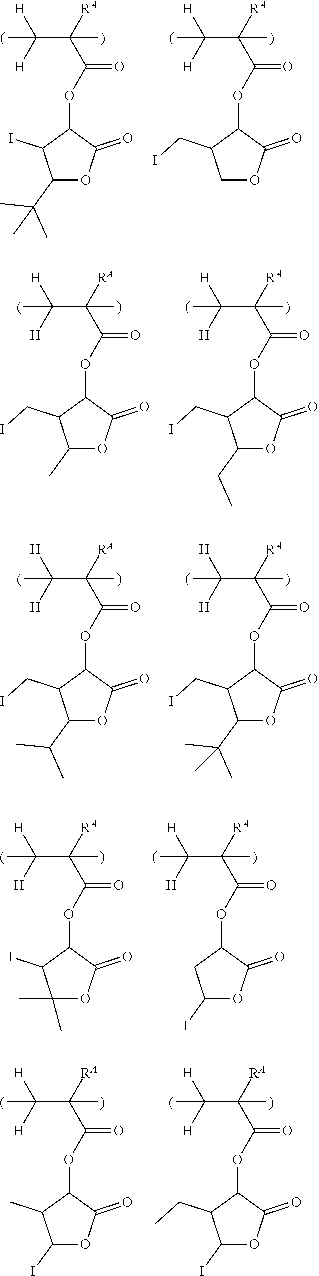

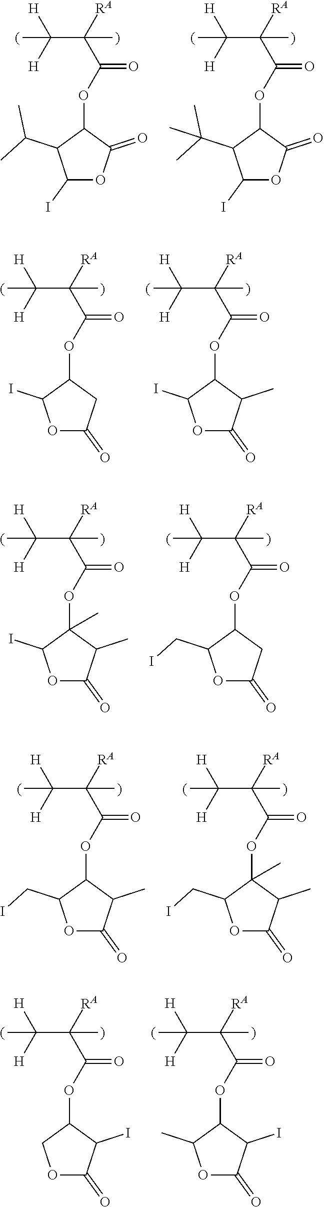

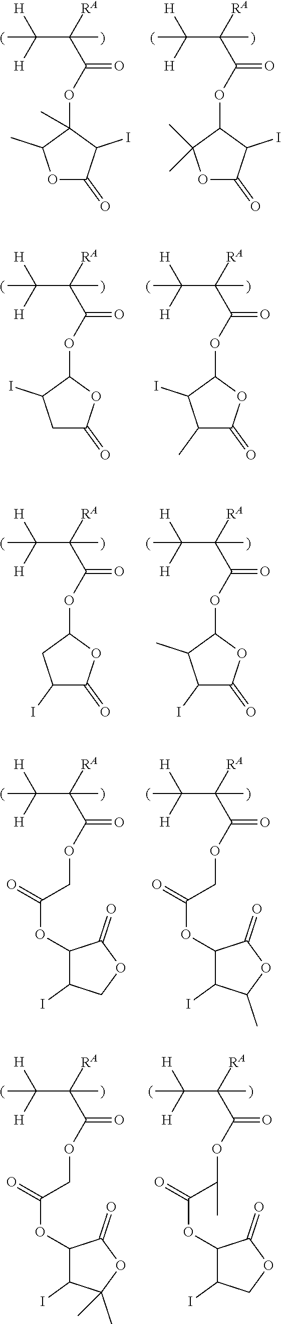

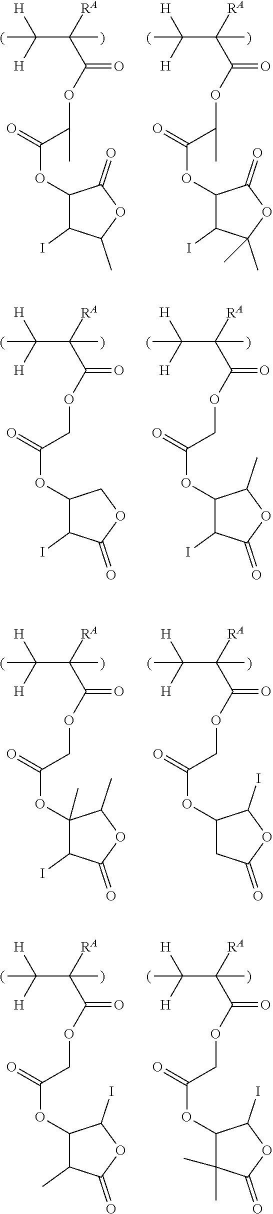

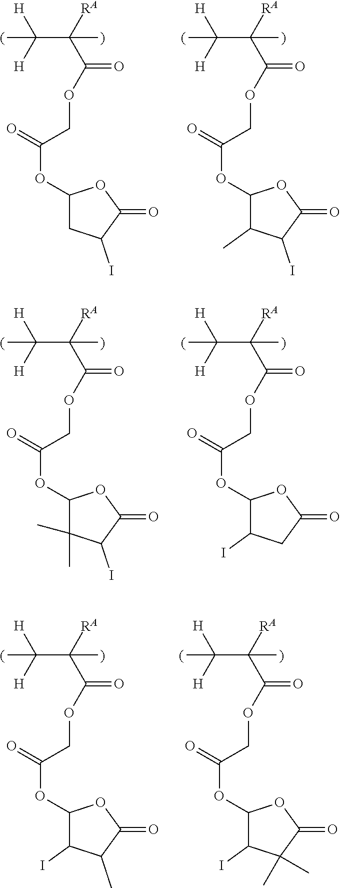

Specific examples of the repeating unit-b2 include, but are not limited to, those shown below. Note that, in the following formulae, R^{A} is as defined above.

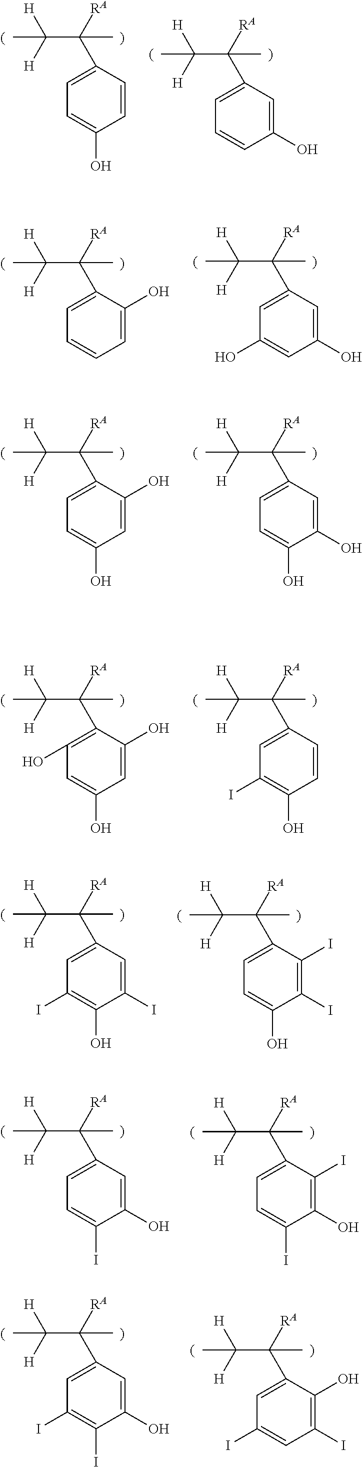

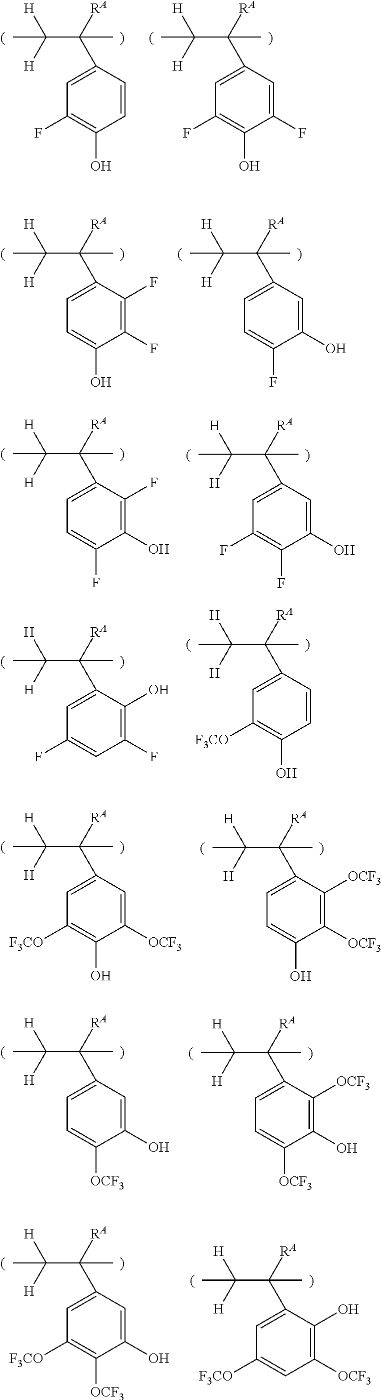

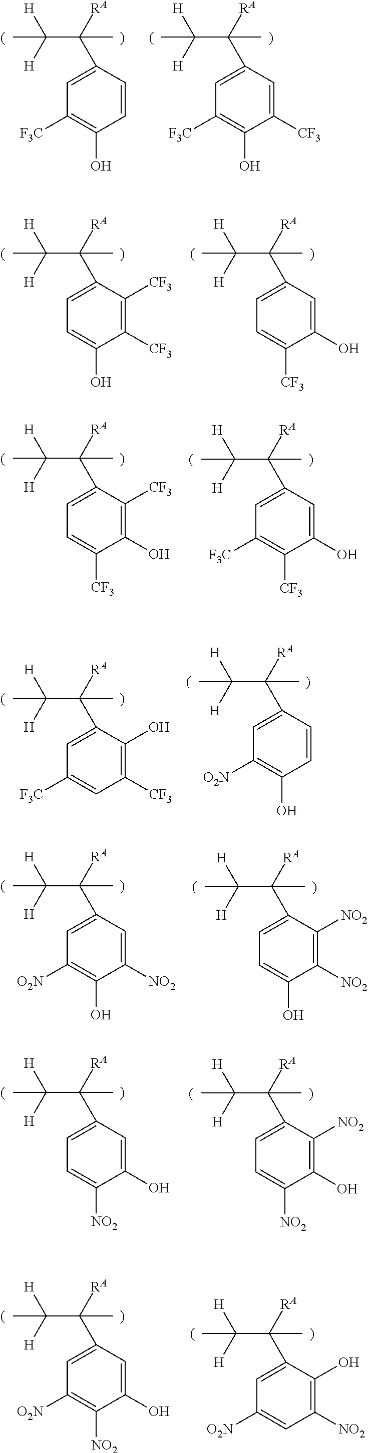

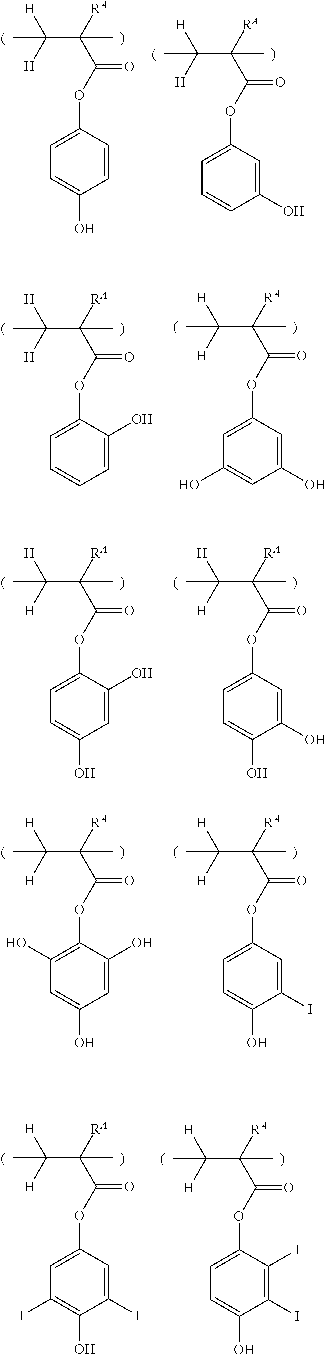

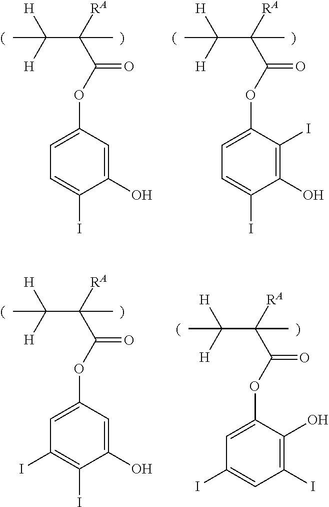

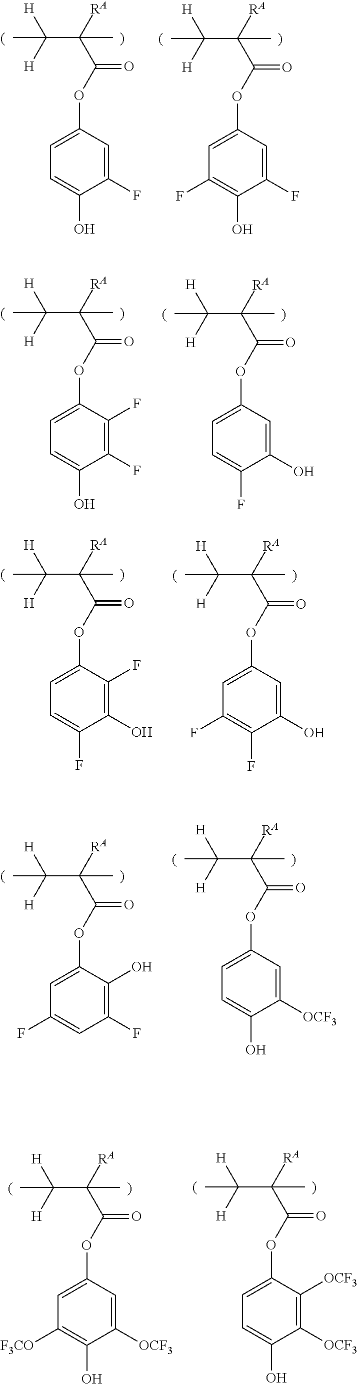

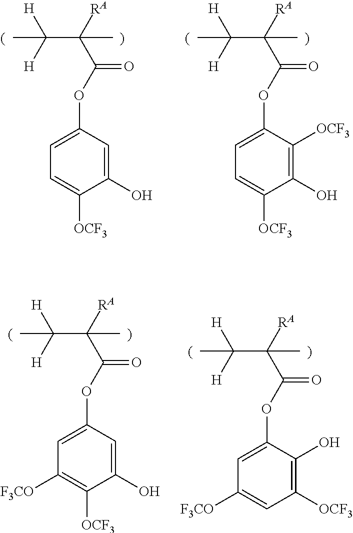

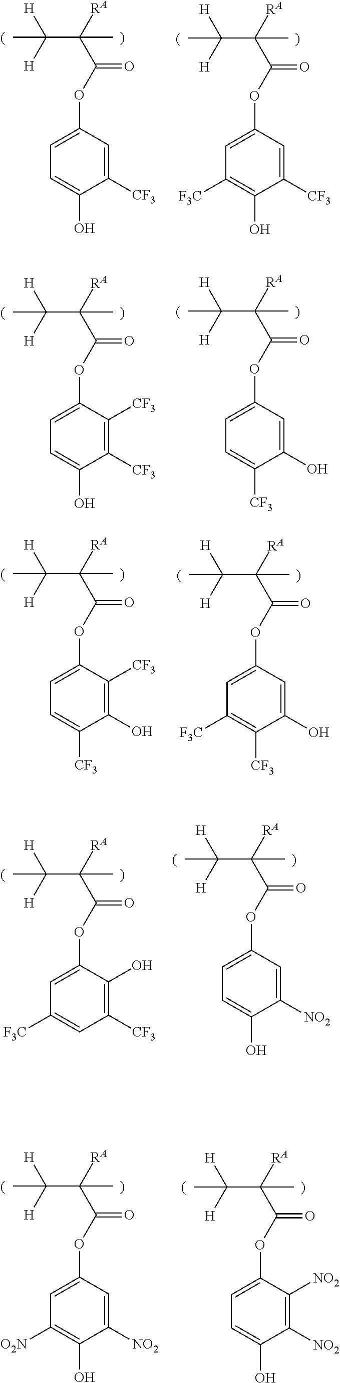

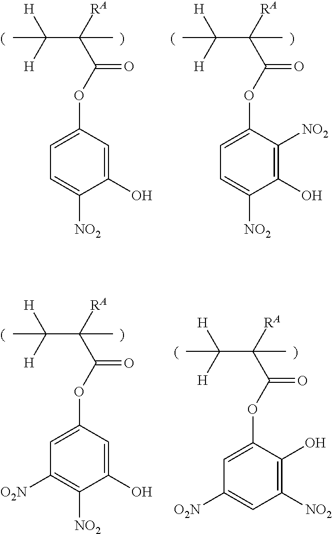

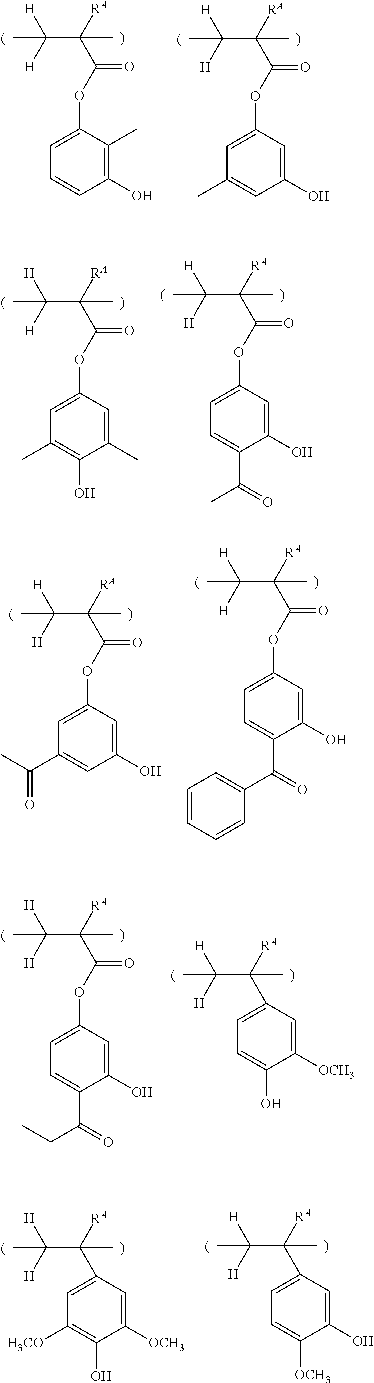

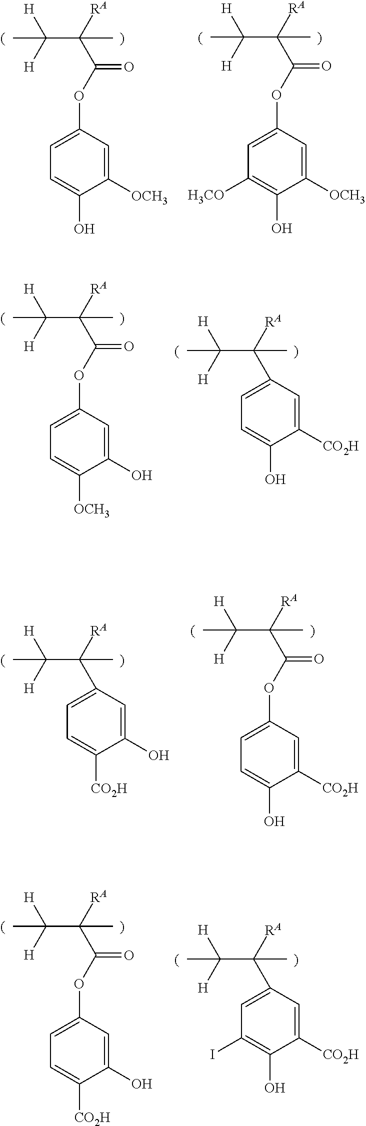

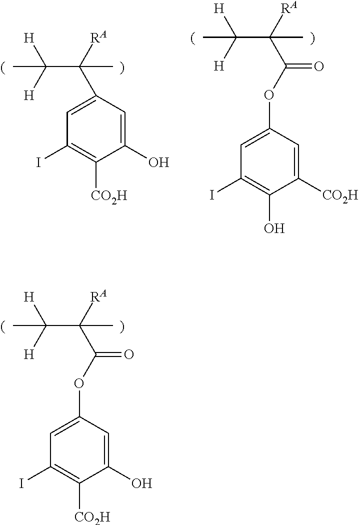

As the repeating unit-bl or b2, in ArF lithography, one having a lactone ring as a polar group is particularly preferable, and in KrF lithography, EB lithography, and EUV lithography, one having a phenol moiety is preferable.

The base polymer (B) preferably further contains at least one constituent selected from a repeating unit represented by the following general formula (c1) (hereinafter also referred to as repeating unit-c1), a repeating unit represented by the following general formula (c2)(hereinafter also referred to as repeating unit-c2), a repeating unit represented by the following general formula (c3)(hereinafter also referred to as repeating unit-c3), a repeating unit represented by the following general formula (c4)(hereinafter also referred to as repeating unit-c4), and a repeating unit represented by the following general formula (c5) (hereinafter also referred to as repeating unit-c5).

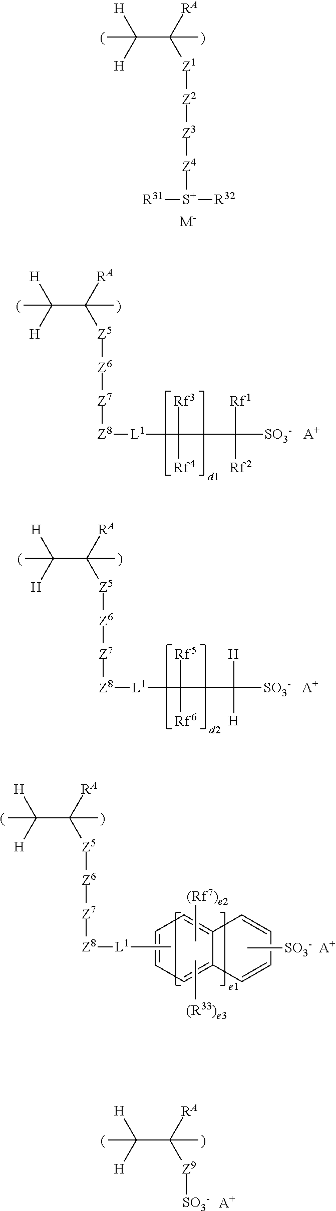

In the formulae, “dl” and “d2” each independently represent an integer from 0 to 3; “e1” is 0 or 1; “e2” is an integer from 0 to 4; “e3” is an integer from 0 to 4; provided that when “e1” is 0, 0≤e2+e354, and when “e1” is 1, 0≤e2+e3≤6;
R^{A} is each independently a hydrogen atom, a fluorine atom, a methyl group, or a trifluoromethyl group;
Z¹ is a single bond, or a phenylene group optionally containing a substituent;
Z² is a single bond, **—C(═O)—O—Z²¹—, **—C(═O)—NH—Z²¹—, or **—O—Z²¹—; Z²¹ is an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ is a single bond, an ether bond, an ester bond, an amide bond, a sulfonate ester bond, a sulfonamide bond, a carbonate bond, or a carbamate bond;
Z⁴ is a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ is each independently a single bond, a phenylene group or a naphthylene group optionally containing a substituent, or *—C(═O)—O—Z⁵¹—; Z⁵¹ is an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ is a single bond, an ether bond, an ester bond, an amide bond, a sulfonate ester bond, a sulfonamide bond, a carbonate bond, or a carbamate bond;
Z⁷ is each independently a single bond, ***—Z⁷¹—C(═O)—O—, ***—C(═O)—NH—Z⁷¹—, or ***—O—Z⁷¹—; Z⁷¹ is a hydrocarbylene group having 1 to 20 carbon atoms and optionally containing a heteroatom;
Z⁸ is each independently a single bond, ****—Z⁸¹—C(═O)—O—, ****—C(═O)—NH—Z⁸¹— or ****—O—Z⁸¹—; Z⁸¹ is a hydrocarbylene group having 1 to 20 carbon atoms and optionally containing a heteroatom;
Z⁰ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹— or *—O—Z⁹¹—; Z⁹¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
“*” represents a bond to a main chain carbon atom; “**” represents a bond to Z¹; “***” represents a bond to Z⁶; “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom, or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms; provided that all Rf⁵ and Rf⁶ are not simultaneously hydrogen atoms;
Rf⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R³¹ and R³² are each independently a hydrocarbyl group having 1 to 20 carbon atoms and optionally containing a heteroatom; also, R³¹ and R³² may be bonded to each other to form a ring together with the sulfur atom to which they are bonded;
R³³ is a halogen atom other than a fluorine atom, or hydrocarbyl group having 1 to 20 carbon atoms and optionally containing a heteroatom; when “e3” is 2, 3, or 4, each R³³ may be the same as or different from each other, and a plurality of R³³ may be bonded to each other to form a ring together with the carbon atoms to which they are bonded;
M⁻ is a non-nucleophilic counter ion; and
A⁺ is an onium cation.

In the general formulae (c1) to (c5), R^{A} is each independently a hydrogen atom, a fluorine atom, a methyl group, or a trifluoromethyl group. Z¹ is a single bond, or a phenylene group optionally containing a substituent. Z² is a single bond, **—C(═O)—O—Z²¹—, **—C(═O)—NH—Z²¹—, or **—O—Z²¹—. Z²¹ is an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group. Z³ is a single bond, an ether bond, an ester bond, an amide bond, a sulfonate ester bond, a sulfonamide bond, a carbonate bond, or a carbamate bond. Z⁴ is a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group. Z⁵ is each independently a single bond, a phenylene group or a naphthylene group optionally containing a substituent, or *—C(═O)—O—Z⁵¹—. Z⁵¹ is an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring. Z⁶ is a single bond, an ether bond, an ester bond, an amide bond, a sulfonate ester bond, a sulfonamide bond, a carbonate bond, or a carbamate bond. Z⁷ is each independently a single bond, ***—Z⁷¹—C(═O)—O—, ***—C(═O)—NH—Z⁷¹—, or ***—O—Z⁷¹—. Z⁷¹ is a hydrocarbylene group having 1 to 20 carbon atoms and optionally containing a heteroatom. Z⁸ is each independently a single bond, ****—Z⁸¹—C(═O)—O—, ****—C(═O)—NH—Z⁸¹—, or ****—O—Z⁸¹—. Z⁸¹ is a hydrocarbylene group having 1 to 20 carbon atoms and optionally containing a heteroatom. Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—. Z⁹¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group. “*” represents a bond to a main chain carbon atom. “**” represents a bond to Z¹. “***” represents a bond to Z⁶. “****” represents a bond to Z⁷.

The aliphatic hydrocarbylene group represented by Z²¹, 251, and Z⁹¹ may be linear, branched, or cyclic, and specific examples thereof include alkanediyl groups such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cycloalkanediyl groups such as a cyclopropanediyl group, a cyclobutanediyl group, a cyclopentanediyl group, and a cyclohexanediyl group; and groups obtained by combining these.

The hydrocarbylene group optionally containing a heteroatom represented by Z⁷¹ and Z⁸¹ may be saturated or unsaturated, and may be linear, branched, or cyclic. Specific examples thereof include, but are not limited to, those shown below.

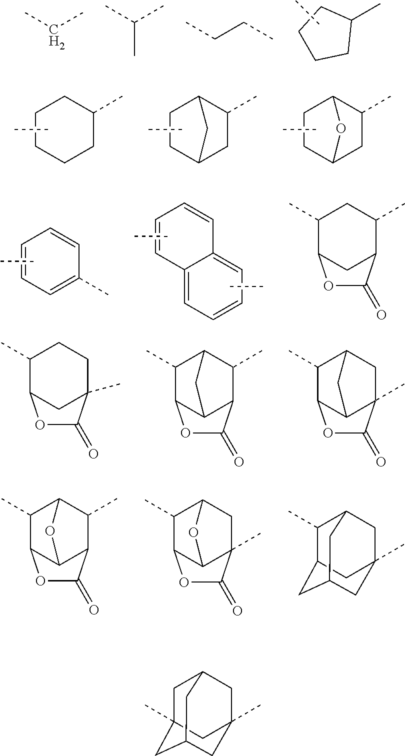

In the formulae, the dashed lines represent bonding points.

In the general formula (c1), R³¹ and R³² are each independently a hydrocarbyl group having 1 to 20 carbon atoms and optionally containing a heteroatom. The hydrocarbyl group may be saturated or unsaturated, and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group, a naphthyl group, and a thienyl group; aralkyl groups having 7 to 20 carbon atoms, such as a benzyl group, a 1-phenylethyl group, and a 2-phenylethyl group; and groups obtained by combining these, and aryl groups are preferred. In addition, some or all of the hydrogen atoms of the hydrocarbyl group may be substituted with a group containing a heteroatom such as an oxygen atom, a sulfur atom, a nitrogen atom, or a halogen atom, and some the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Also, R³¹ and R³² may be bonded to each other to form a ring together with the sulfur atom to which they are bonded. In this case, specific examples of the ring include those represented by the following formulae.

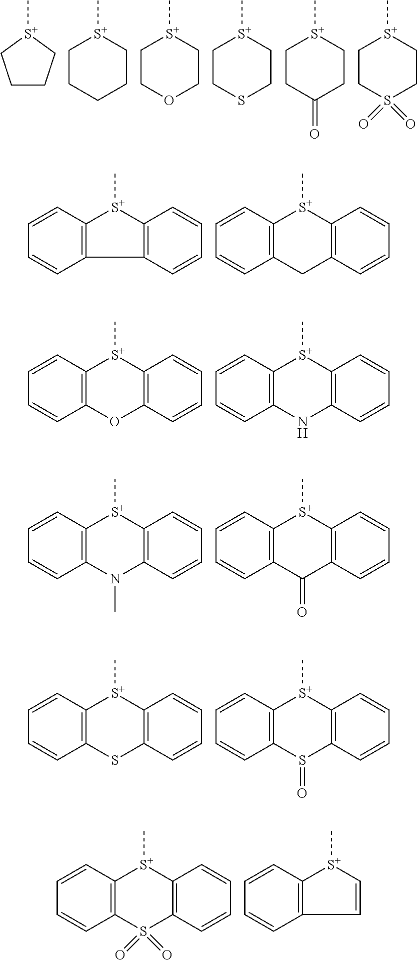

In the formulae, the dashed line indicates the bonding point to Z⁴.

Specific examples of the cation of the repeating unit-c1 include, but are not limited to, those shown below. Note that, in the following formulae, R^{A} is as defined above.

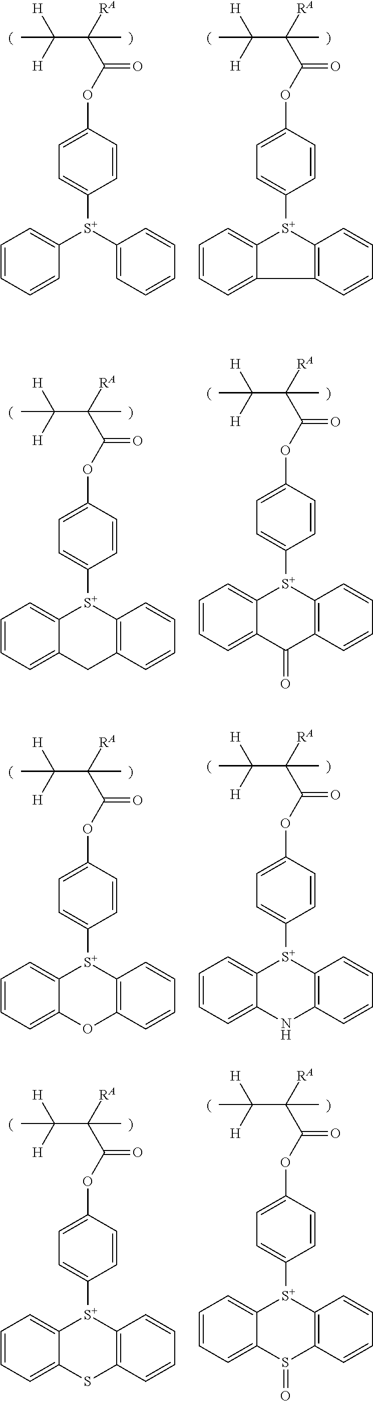

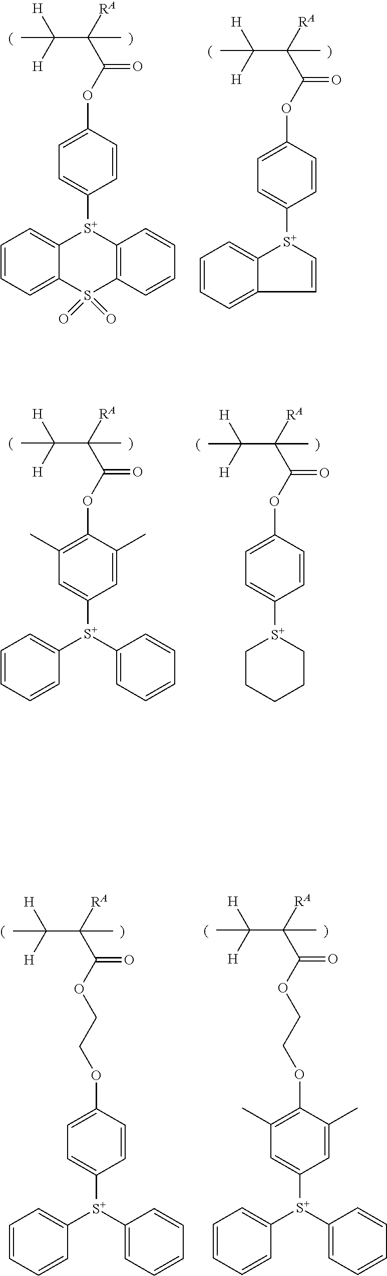

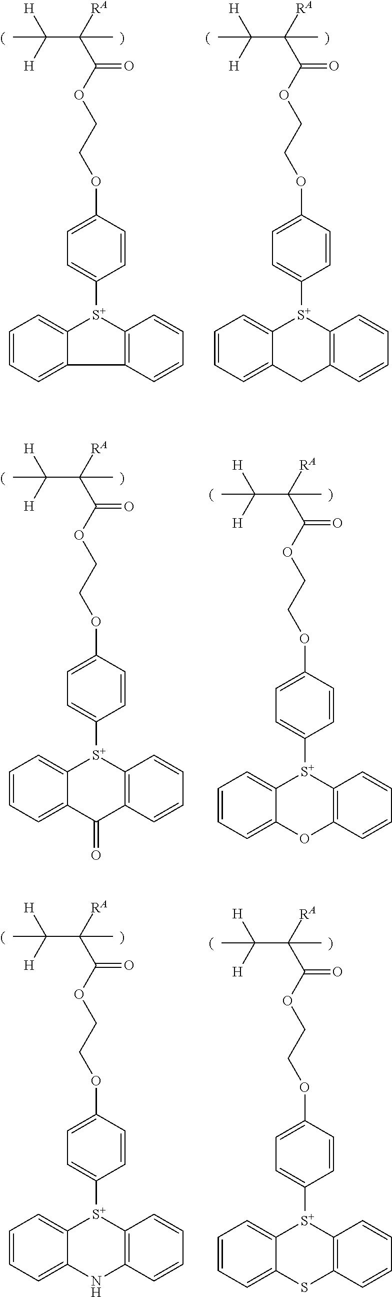

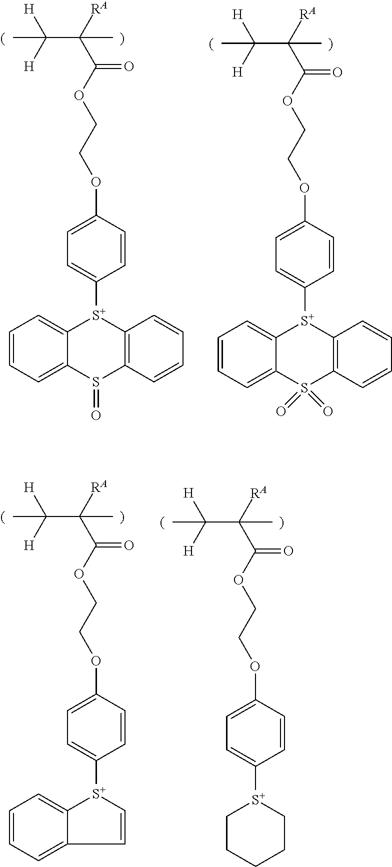

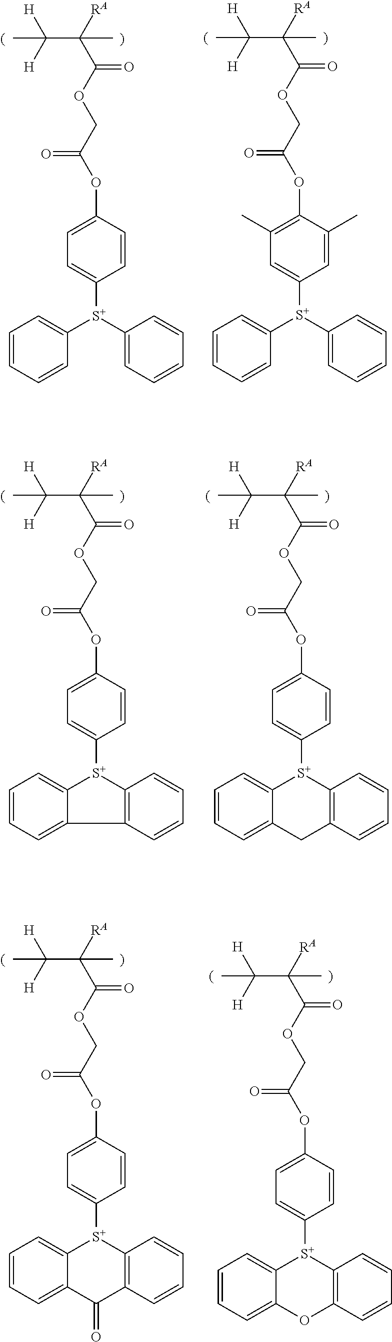

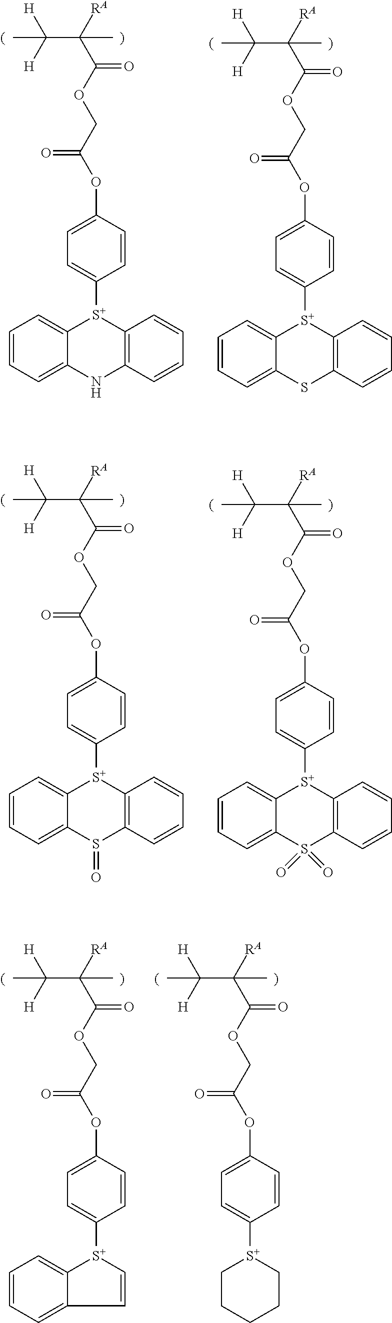

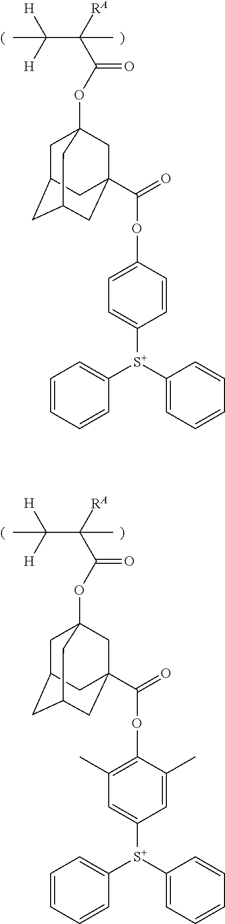

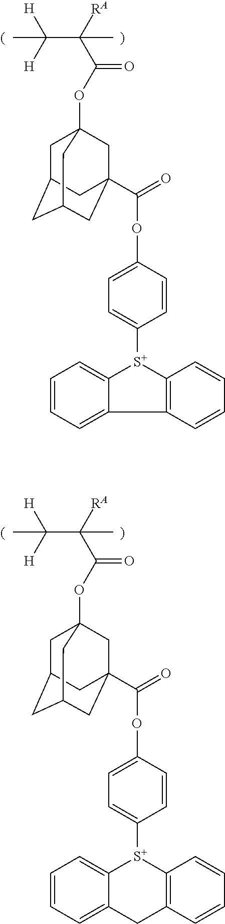

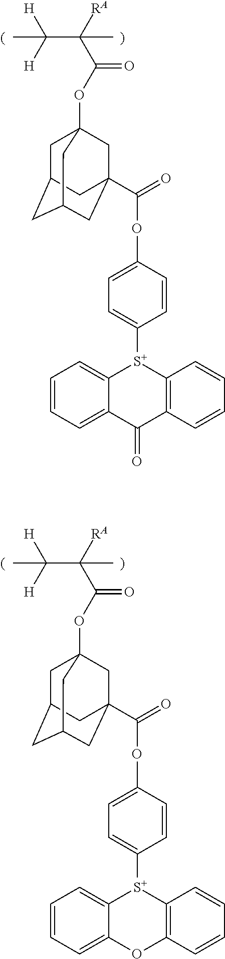

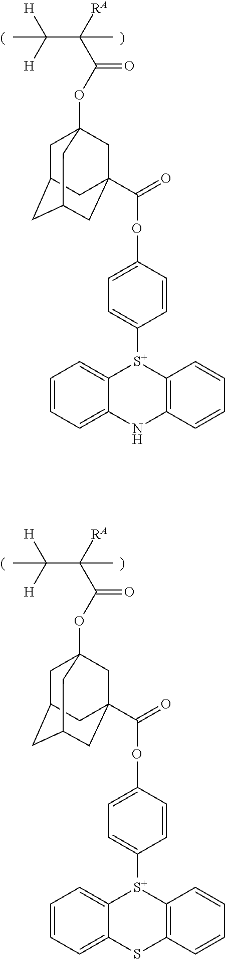

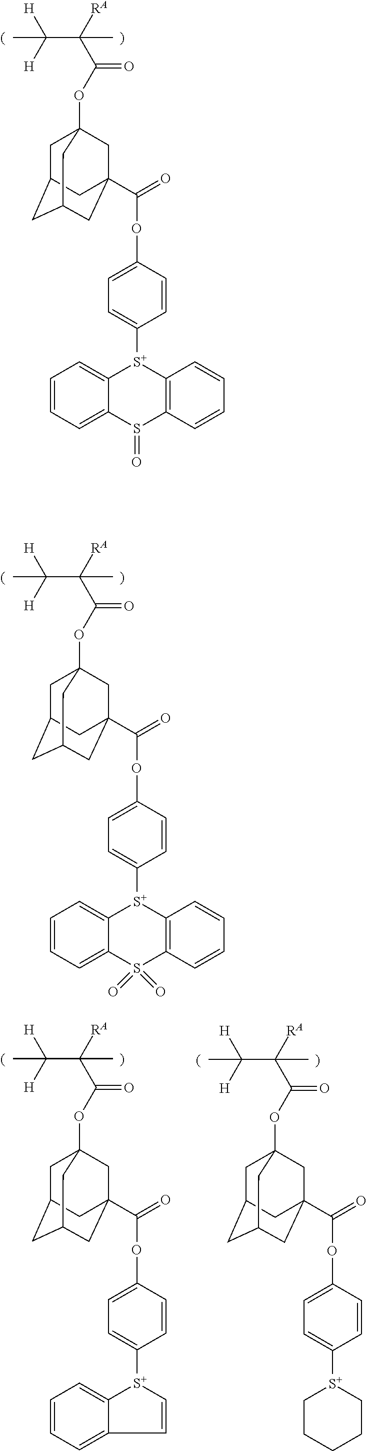

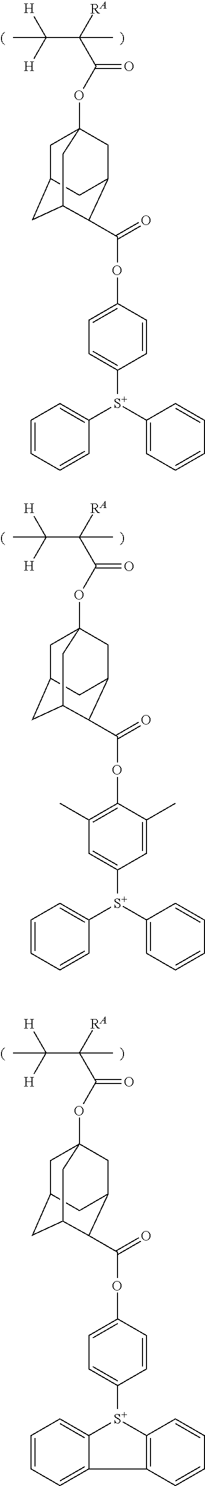

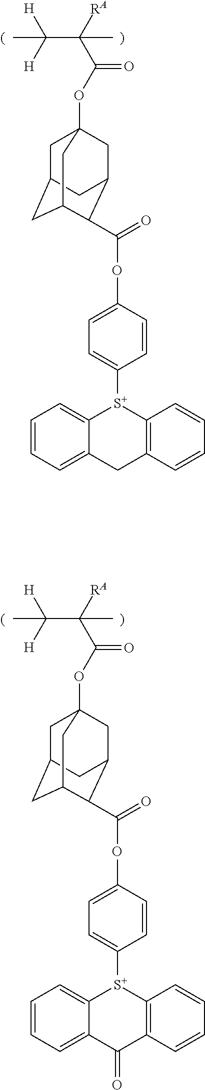

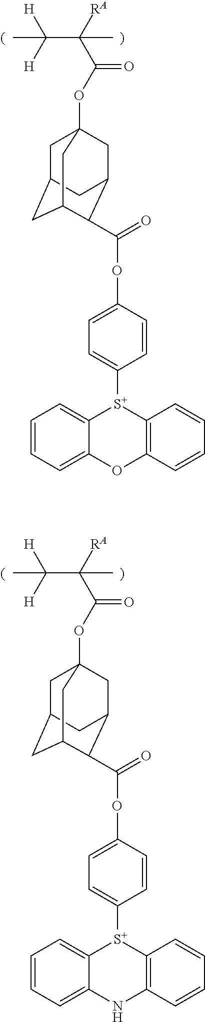

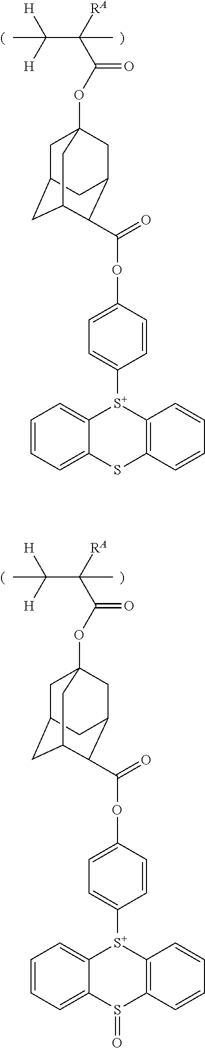

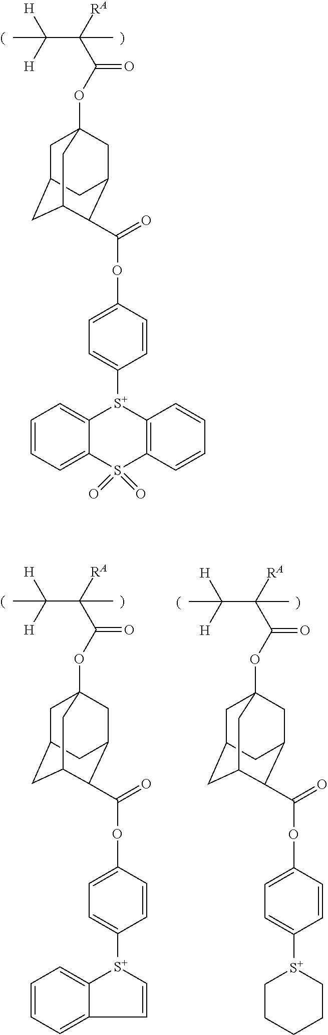

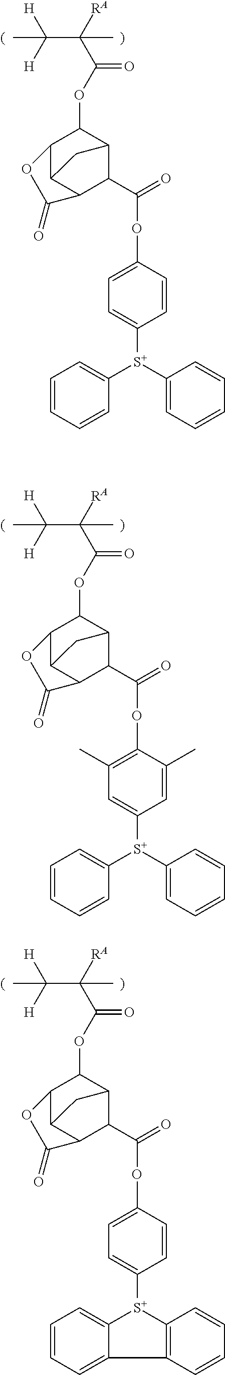

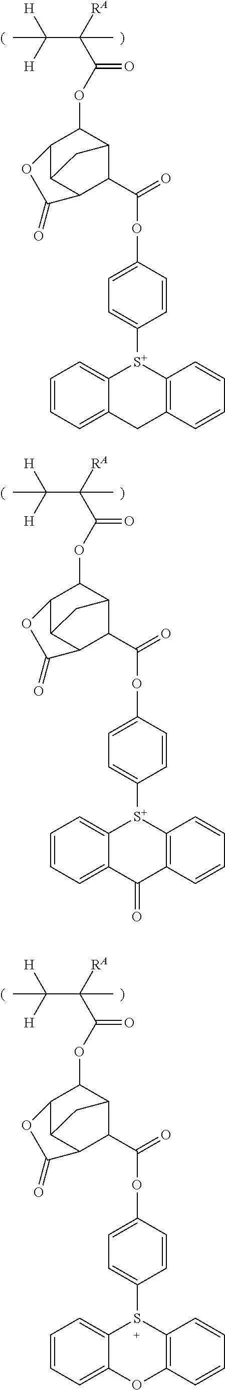

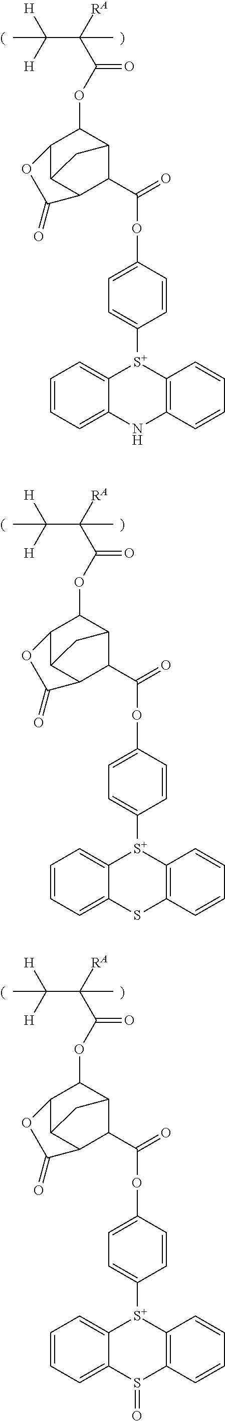

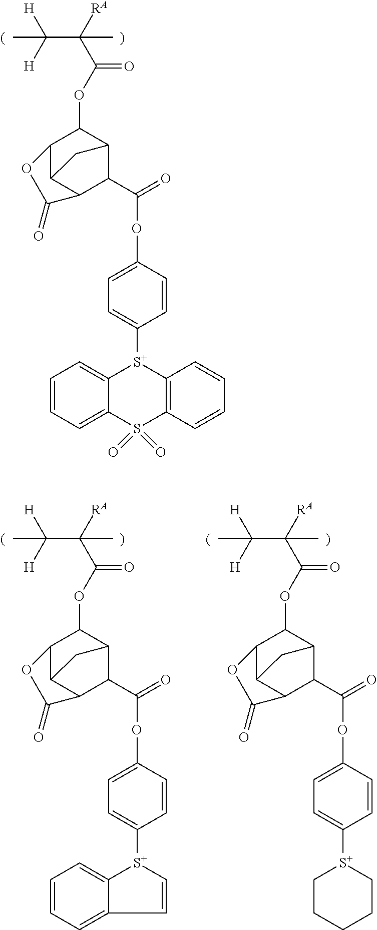

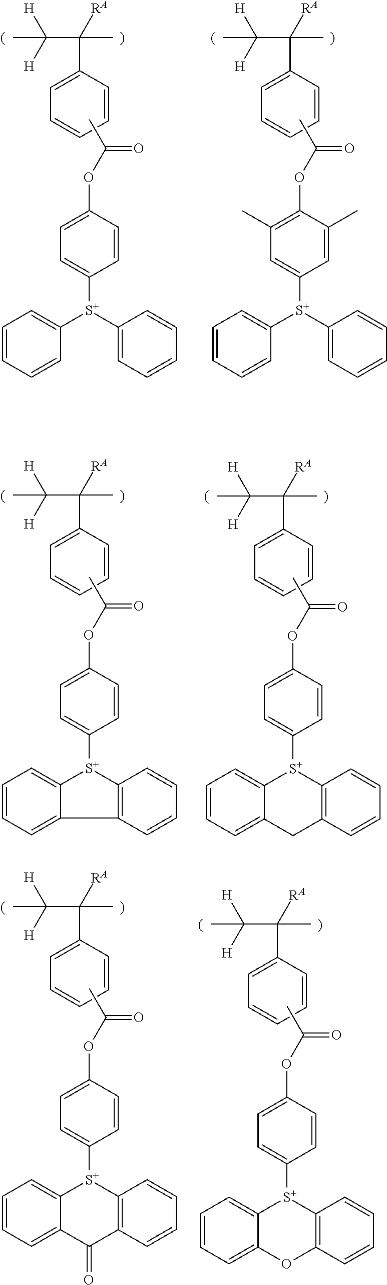

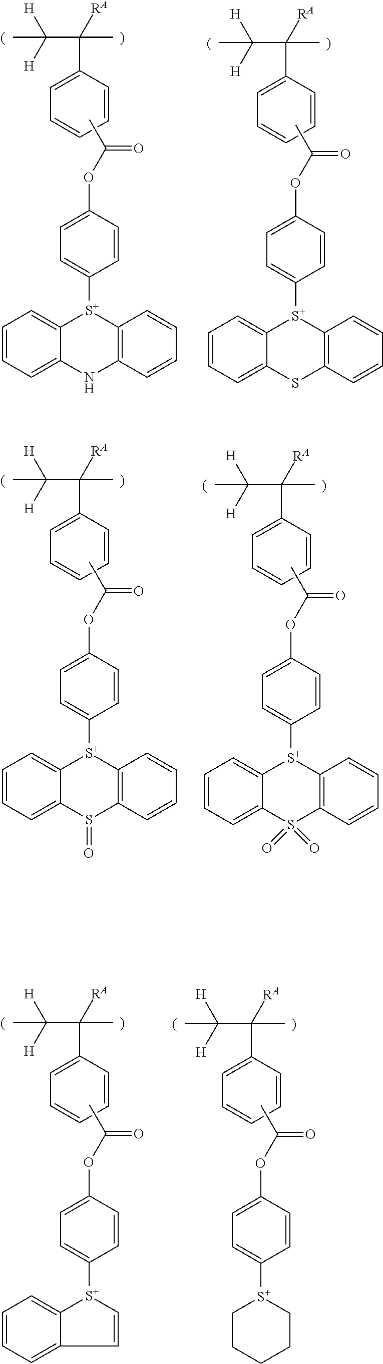

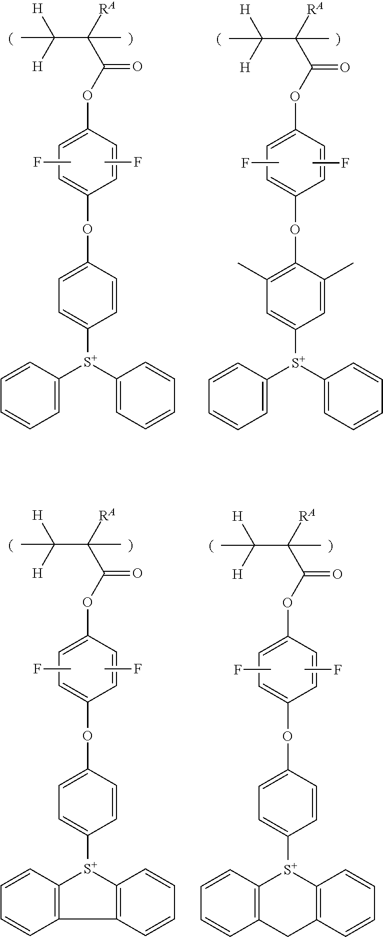

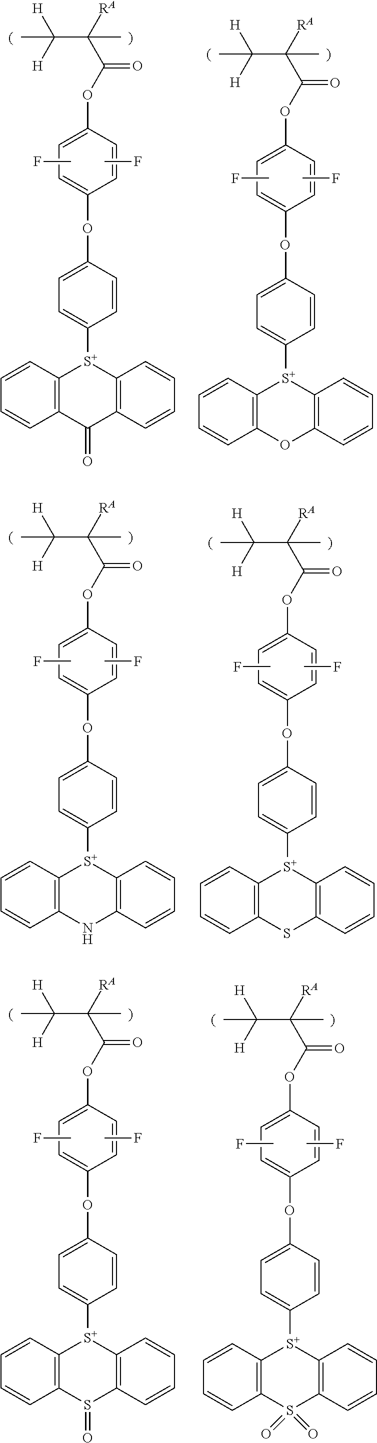

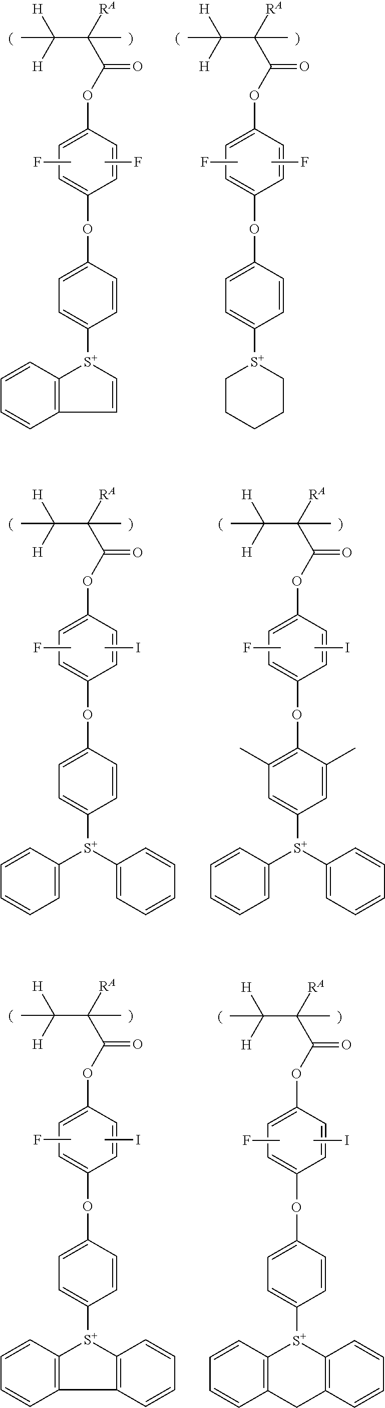

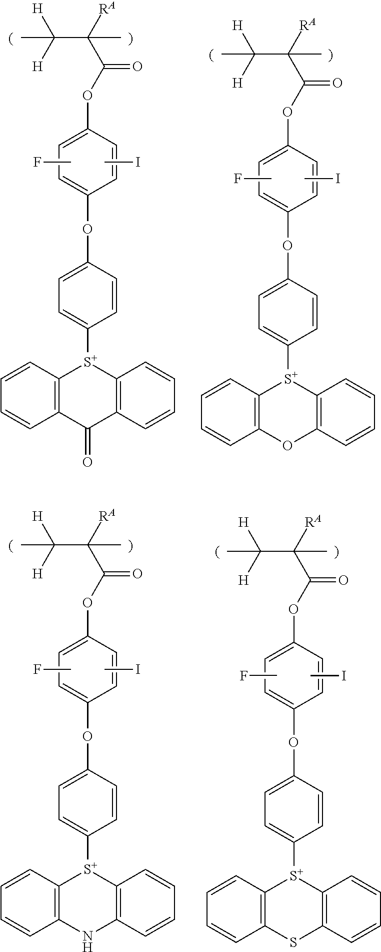

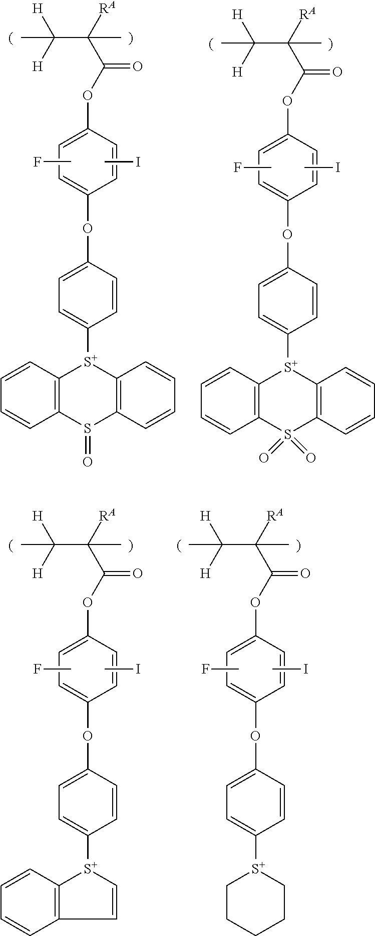

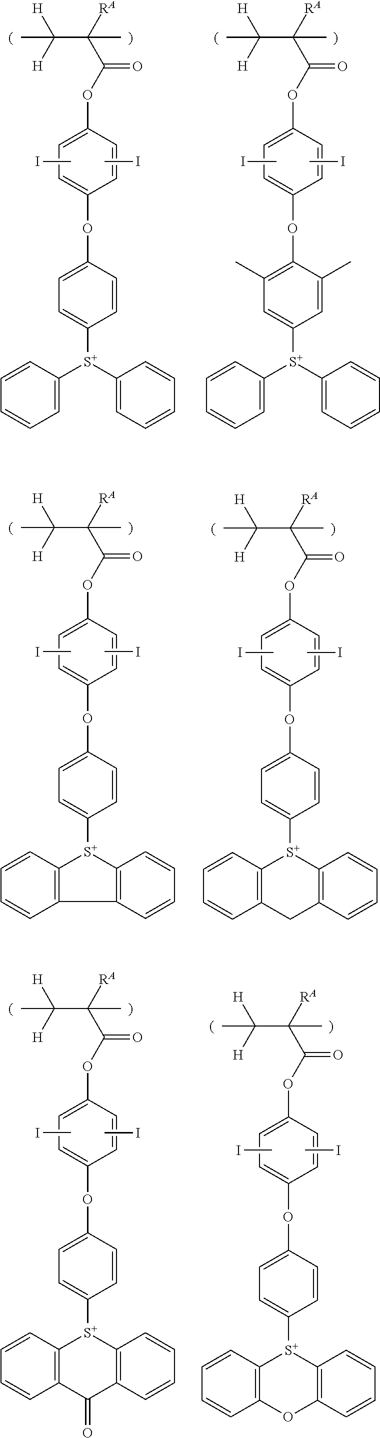

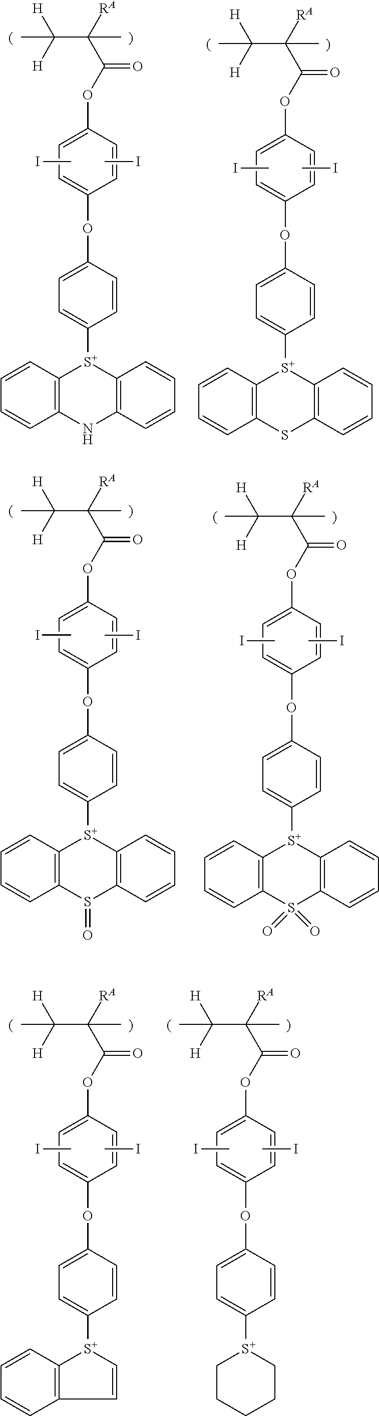

In the general formula (c1), M⁻ is a non-nucleophilic counterion. As the non-nucleophilic counterion, a halide ion, a sulfonate anion, an imidate anion, and a methide anion are preferable. Specific examples of the halide ion include a chloride ion, a bromide ion, etc. Specific examples of the sulfonate anion (sulfonate ion) include fluoroalkylsulfonate ions such as a triflate ion, a 1,1,1-trifluoroethanesulfonate ion, and a nonafluorobutanesulfonate ion; arylsulfonate ions such as a tosylate ion, a benzenesulfonate ion, a 4-fluorobenzenesulfonate ion, and a 1,2,3,4,5-pentafluorobenzenesulfonate ion; and alkylsulfonate ions such as a mesylate ion and a butanesulfonate ion. Specific examples of the imidate anion (imide ion) include a bis(trifluoromethylsulfonyl)imide ion, a bis(perfluoroethylsulfonyl)imide ion, and a bis(perfluorobutylsulfonyl)imide ion. Specific examples of the methide acid anion (methide ion) include a tris(trifluoromethylsulfonyl) methide ion, and a tris(perfluoroethylsulfonyl) methide ion.

Other examples of the non-nucleophilic counterion include anions represented by any of the following general formulae (c1-1) to (c1-4).

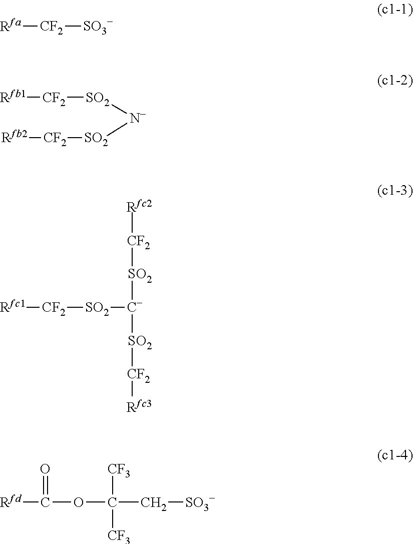

In the above general formula (c1-1), R^{fa} is a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (c1-1-1) described later.

The anion represented by general formula (c1-1) is preferably one represented by the following general formula (c1-1-1).

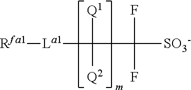

In the general formula (c1-1-1), Q1 and 02 are each independently a hydrogen atom, a fluorine atom, or a fluorinated saturated hydrocarbyl group having 1 to 6 carbon atoms, and at least one of them is preferably a trifluoromethyl group, in order to improve solvent solubility. “m” is an integer from 0 to 4, and is particularly preferably 1. R^{fa1} is a hydrocarbyl group having 1 to 35 carbon atoms and optionally containing a heteroatom. As the heteroatom, an oxygen atom, a nitrogen atom, a sulfur atom, a halogen atom, etc. are preferable, and an oxygen atom is more preferable. As the hydrocarbyl group, from the viewpoint of obtaining high resolution in fine pattern formation, ones having 6 to 30 carbon atoms are particularly preferable.

In the general formula (c1-1-1), the hydrocarbyl group having 1 to 35 carbon atoms and represented by R^{fa1} may be saturated or unsaturated, and may be linear, branched, or cyclic. Specific examples thereof include alkyl groups having 1 to 35 carbon atoms, such as a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups having 3 to 35 carbon atoms, such as a cyclopentyl group, a cyclohexyl group, a 1-adamantyl group, a 2-adamantyl group, a 1-adamantyl methyl group, a norbornyl group, a norbornyl methyl group, a tricyclodecyl group, a tetracyclododecyl group, a tetracyclododecylmethyl group, and a dicyclohexylmethyl group; unsaturated aliphatic hydrocarbyl groups having 2 to 35 carbon atoms, such as a 2-propenyl group and a 3-cyclohexenyl group; aryl groups having 6 to 35 carbon atoms, such as a phenyl group, a 1-naphthyl group, a 2-naphthyl group, and a 9-fluorenyl group; aralkyl groups having 7 to 35 carbon atoms, such as a benzyl group and a diphenylmethyl group; and groups obtained by combining these.

In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. Specific examples of hydrocarbyl groups containing heteroatoms include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

In the general formula (c1-1-1), Lal is a single bond, an ether bond, an ester bond, a sulfonate ester bond, a carbonate bond, or a carbamate bond, but from the viewpoint of synthesis, it is preferably an ether bond or an ester bond, and more preferably an ester bond.

Specific examples of the anion represented by the general formula (c1-1) include, but are not limited to, those shown below. Note that, in the following formulae, Q1 is as defined above, and Ac is an acetyl group.

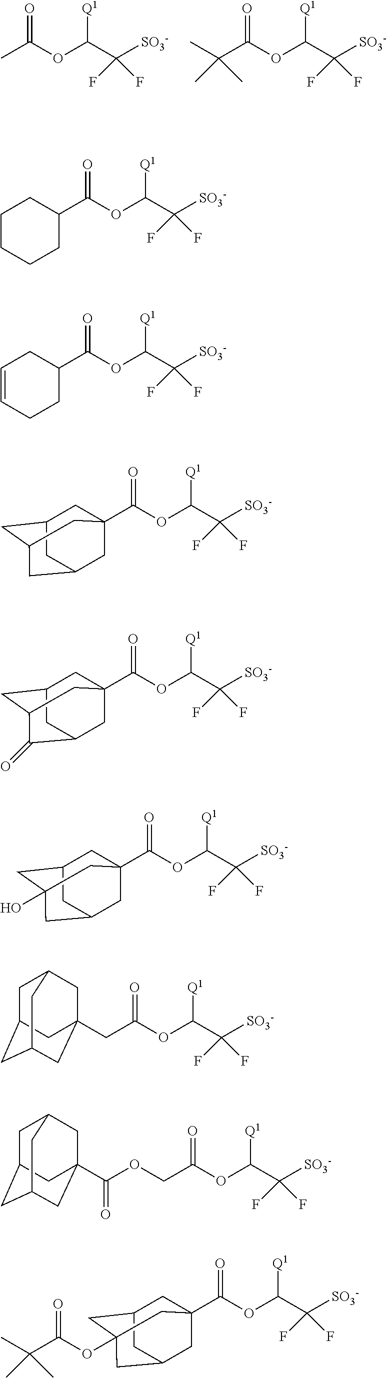

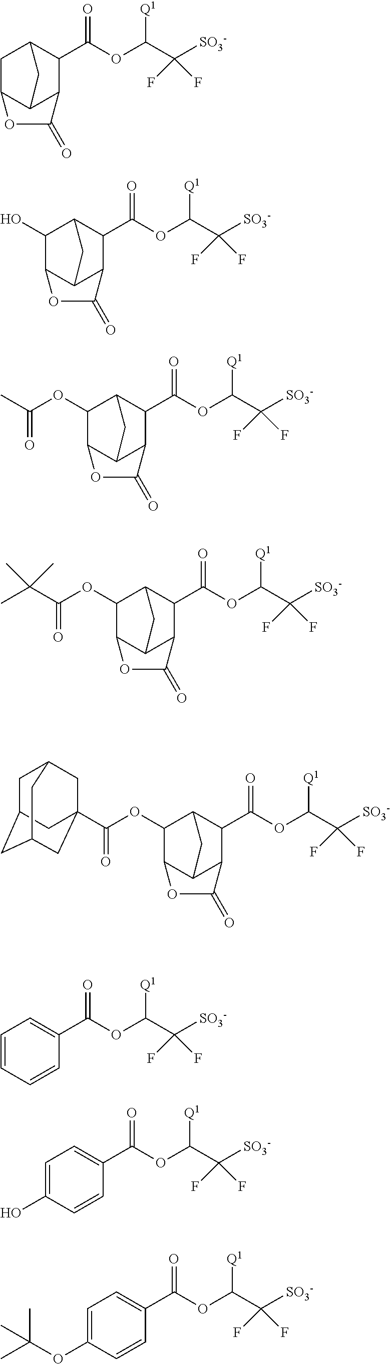

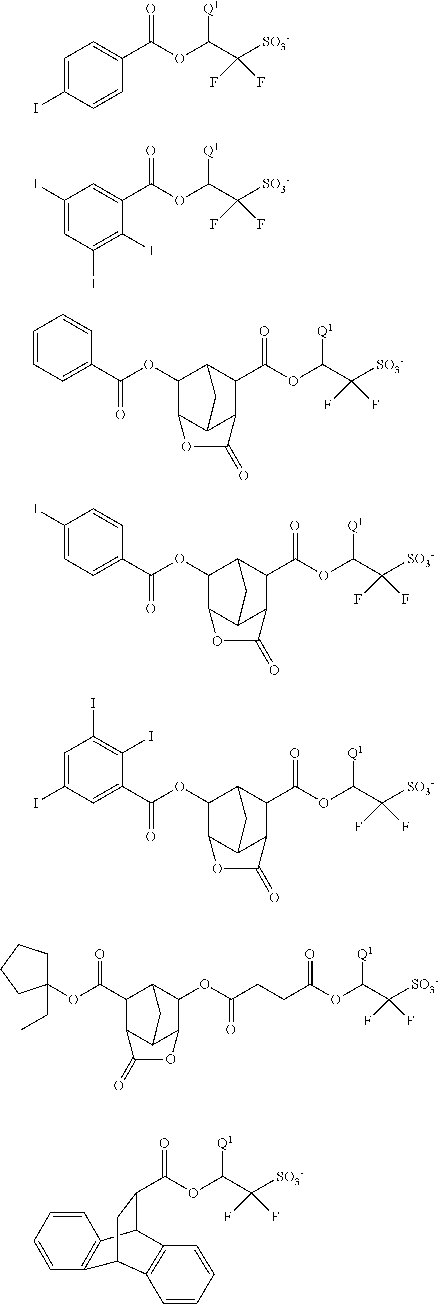

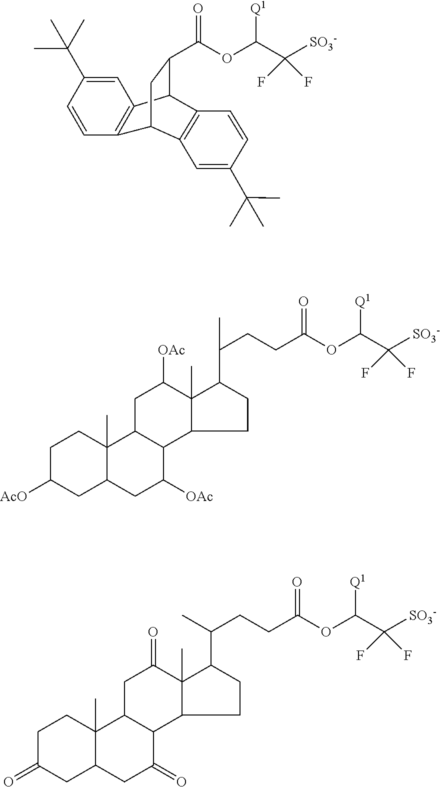

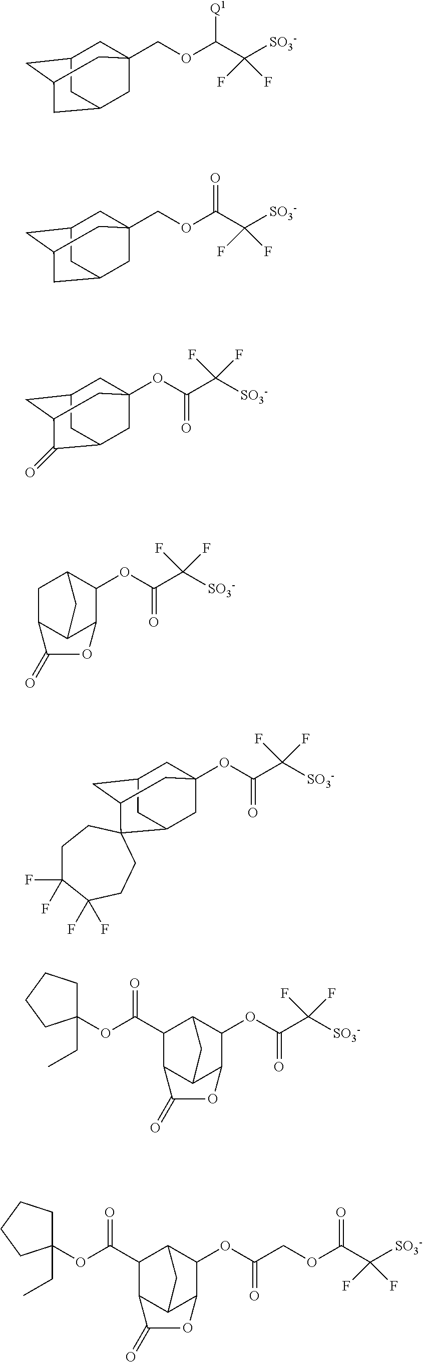

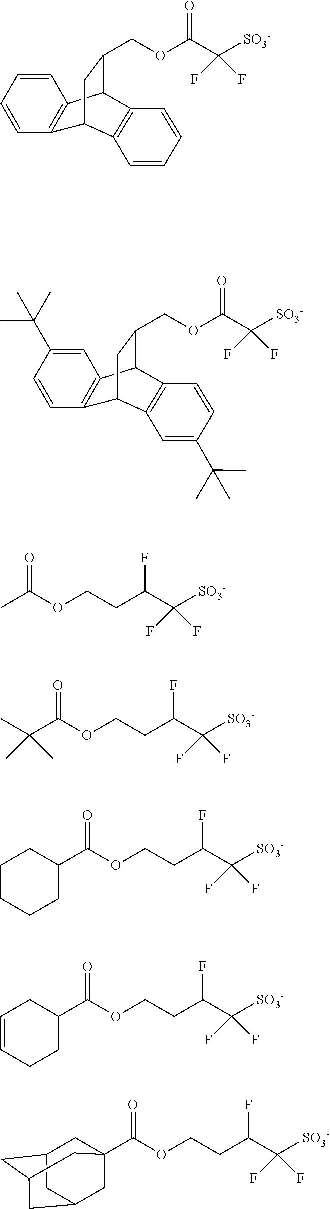

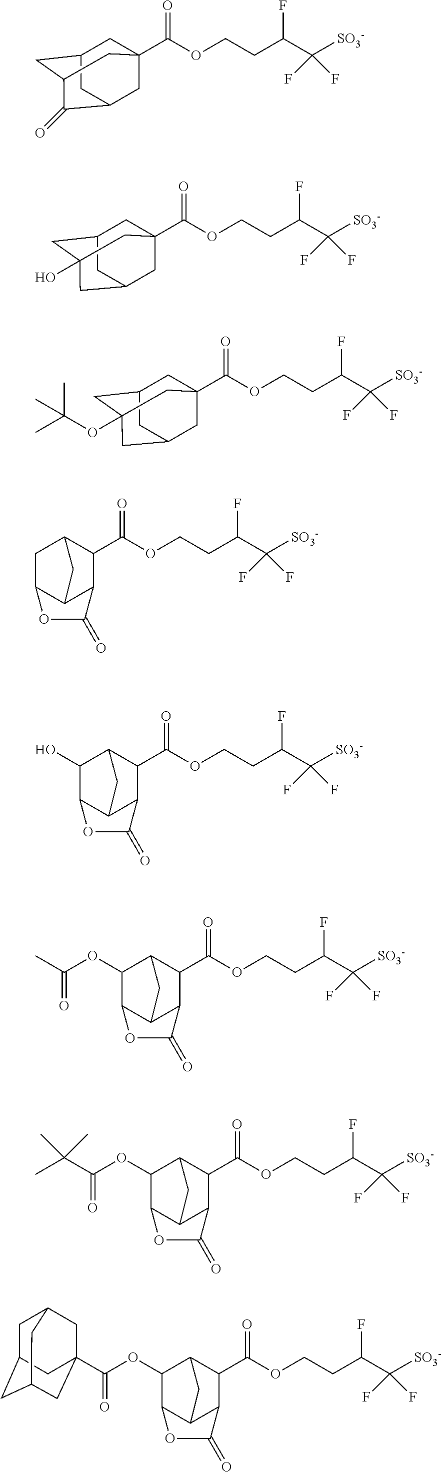

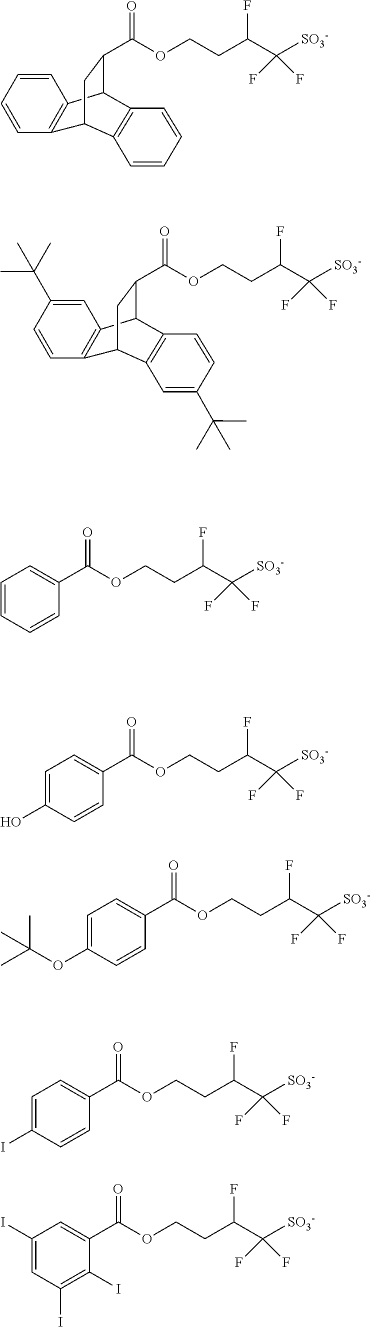

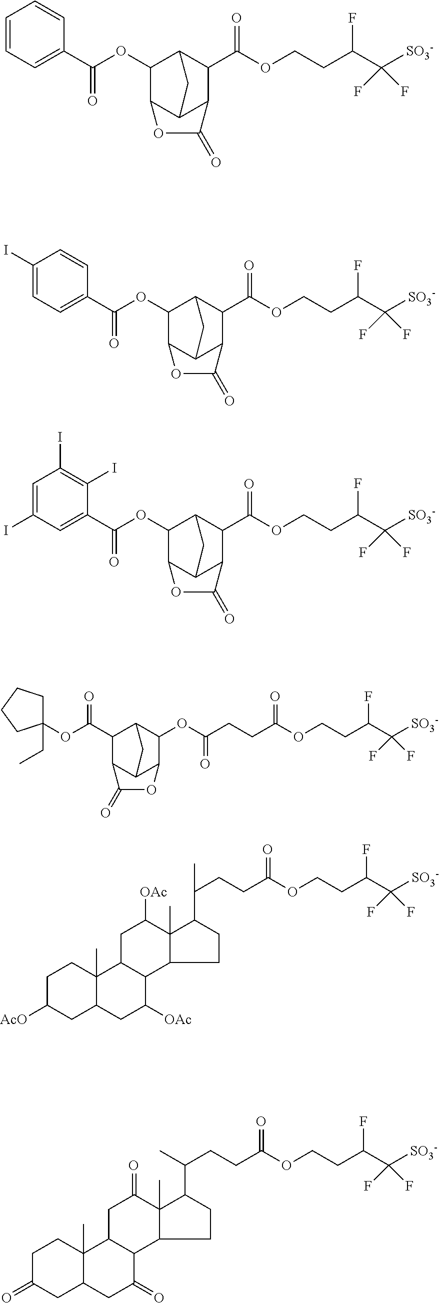

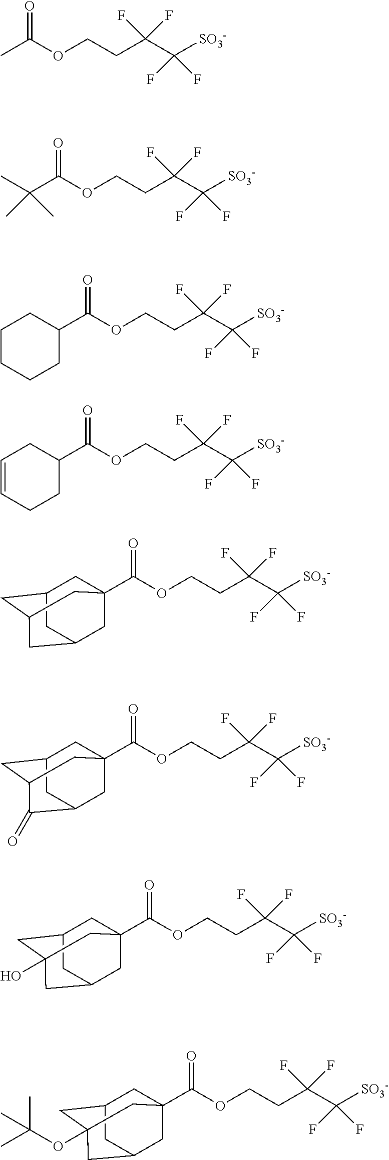

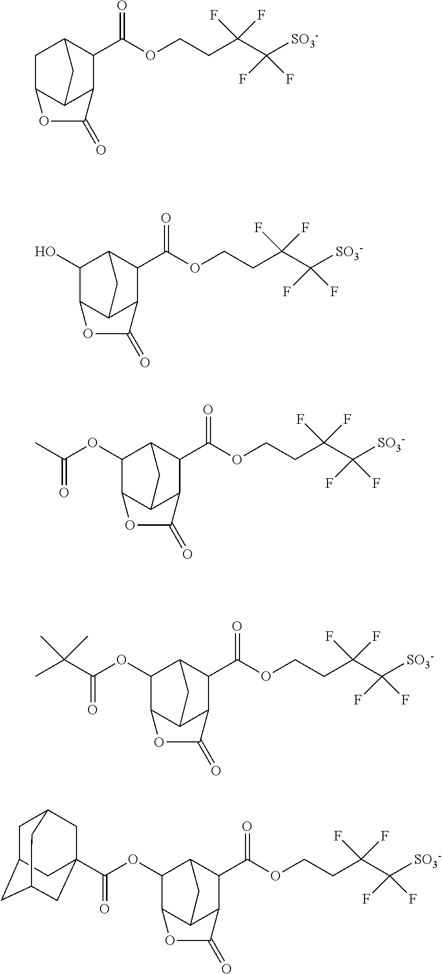

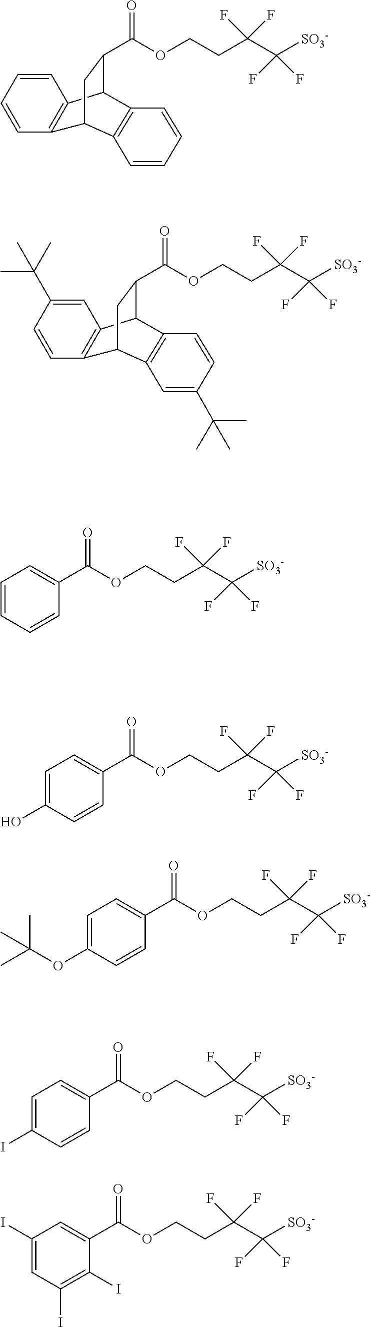

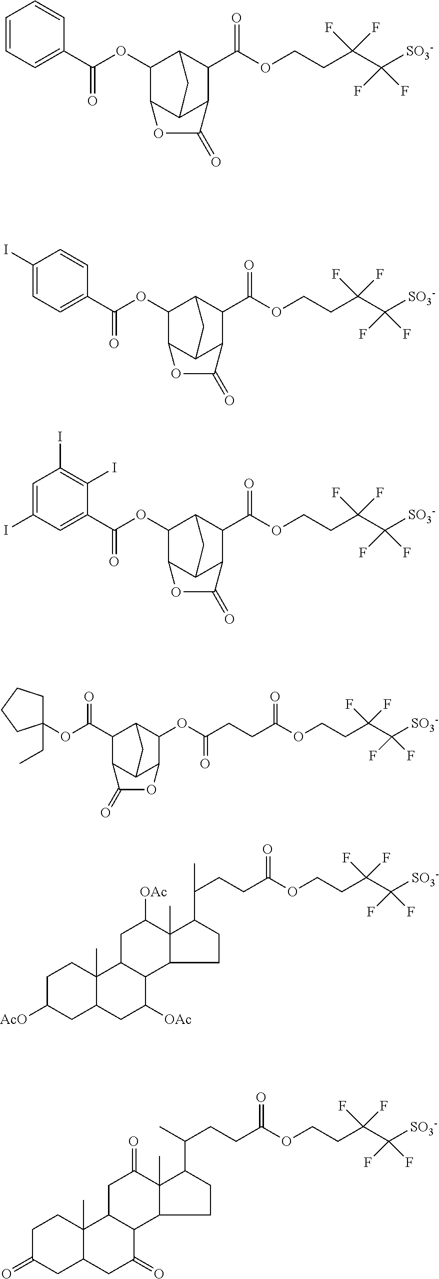

In the general formula (c1-2), R^{fb1} and R^{fb2} are each independently a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (c1-1-1). R^{fb1} and R^{fb2} are preferably a fluorine atom or a linear fluorinated alkyl group having 1 to 4 carbon atoms. In addition, R^{fb1} and R^{fb2} may bonded to each other to form a ring together with the group (—CF₂—SO₂—N⁻—SO₂—CF₂—) to which they are bonded. In this case, the group obtained by R^{fb1} and R^{fb2} being bonded to each other is preferably a fluorinated ethylene group or a fluorinated propylene group.

In the general formula (c1-3), R^{fc1}, R^{fc2}, and R^{fc3} are each independently a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (c1-1-1). R^{fc1}, R^{fc2}, and R^{fc3} are preferably a fluorine atom or a linear fluorinated alkyl group having 1 to 4 carbon atoms. In addition, R^{fc1} and R^{fc2} may be bonded to each other to form a ring together with the group (—CF₂—SO₂—C⁻—SO₂—CF₂—) to which they are bonded. In this case, the group obtained by R^{fc1} and R^{fc2} being bonded to each other is preferably a fluorinated ethylene group or a fluorinated propylene group.

In the general formula (c1-4), R^{fd} is a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (c1-1-1).

Specific examples of the anion represented by the general formula (c1-4) include, but are not limited to, those shown below.

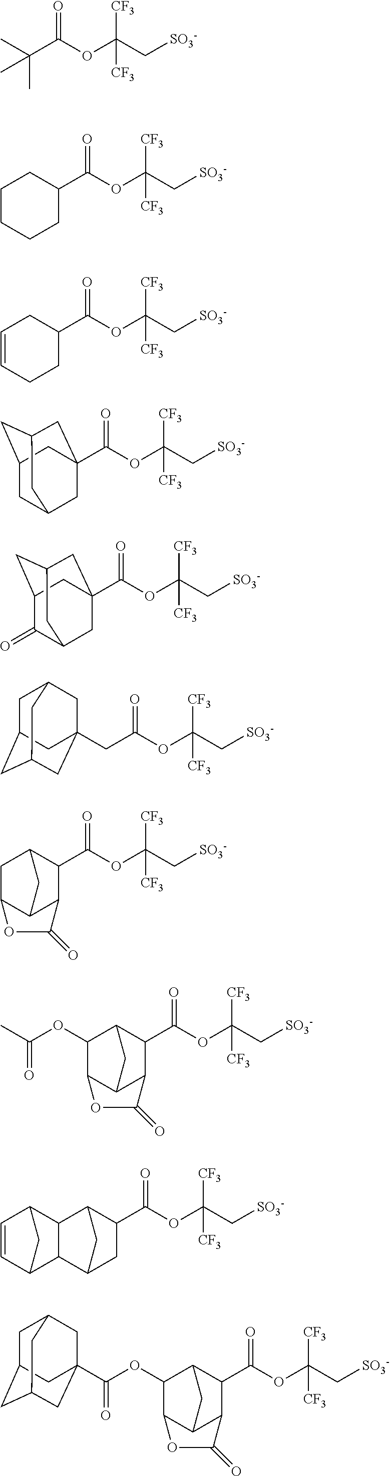

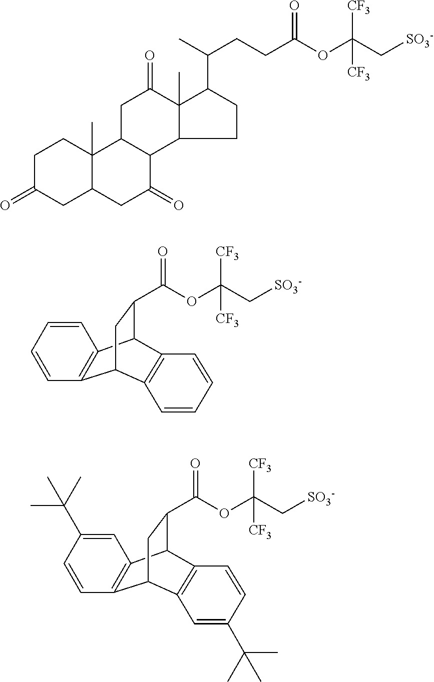

Examples of the non-nucleophilic counterion further include an anion having an aromatic ring substituted with an iodine atom or a bromine atom. Specific examples of such an anion include those represented by the following general formula (c1-5).

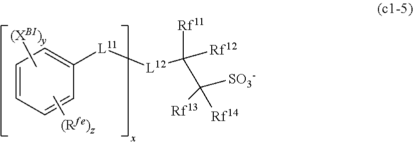

In the general formula (c1-5), “x” is 1, 2, or 3. “y” is an integer from 1 to 5. “z” is an integer from 0 to 3. Note, 1≤y+z≤5. “y” is preferably 1, 2, or 3, and more preferably 2 or 3. “z” is preferably 0, 1, or 2.

In the general formula (c1-5), X^{BI} is an iodine atom or a bromine atom, and may be the same as or different from each other when “x” and/or “y” are 2 or more.

In the general formula (c1-5), L¹¹ is a single bond, an ether bond, an ester bond, or a saturated hydrocarbylene group having 1 to 6 carbon atoms and optionally containing an ether bond or an ester bond. The saturated hydrocarbylene group may be linear, branched, or cyclic.

In the general formula (c1-5), when “x” is 1, 112 is a single bond or a divalent linking group having 1 to 20 carbon atoms, and when “x” is 2 or 3, L¹² is a (x+1)-valent linking group having 1 to 20 carbon atoms, and the linking group may contain an oxygen atom, a sulfur atom, or a nitrogen atom.

In the general formula (c1-5), R^{fe} is a hydroxy group, a carboxy group, a fluorine atom, a chlorine atom, a bromine atom, or an amino group, or a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, or a hydrocarbylsulfonyloxy group having 1 to 20 carbon atoms and optionally containing a fluorine atom, a chlorine atom, a bromine atom, a hydroxy group, an amino group, or a ether bond, or —N(R^{feA})(R^{feB}), —N(R^{feC})—C(═O)—R^{feD}, or —N(R^{feC})—C(═O)—O—R^{feD}. R^{feA} and R^{feB} are each independently a hydrogen atom or a saturated hydrocarbyl group having 1 to 6 carbon atoms. R^{feC} is a hydrogen atom or a saturated hydrocarbyl group having 1 to 6 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{feD} is an aliphatic hydrocarbyl group having 1 to 16 carbon atoms, an aryl group having 6 to 12 carbon atoms, or an aralkyl group having 7 to 15 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When “x” and/or “z” are 2 or more, each R^{fe} may be the same as or different from each other.

Of these, preferred examples of R^{fe} include a hydroxy group, —N(R^{feC})—C(═O)—R^{feD}, —N(R^{feC})—C(═O)—O—R^{feD}, a fluorine atom, a chlorine atom, a bromine atom, a methyl group, and a methoxy group.

In the general formula (c1-5), Rf¹¹ to Rf¹⁴ are each independently a hydrogen atom, a fluorine atom, or a trifluoromethyl group, and at least one of them is a fluorine atom or a trifluoromethyl group. In addition, Rf¹¹ and Rf¹² may be combined to form a carbonyl group. In particular, Rf¹³ and Rf¹⁴ are preferably both fluorine atoms.

Specific examples of the anion represented by the general formula (c1-5) include, but are not limited to, those shown below. Note that, in the following formulae, X^{BI} is as defined above.

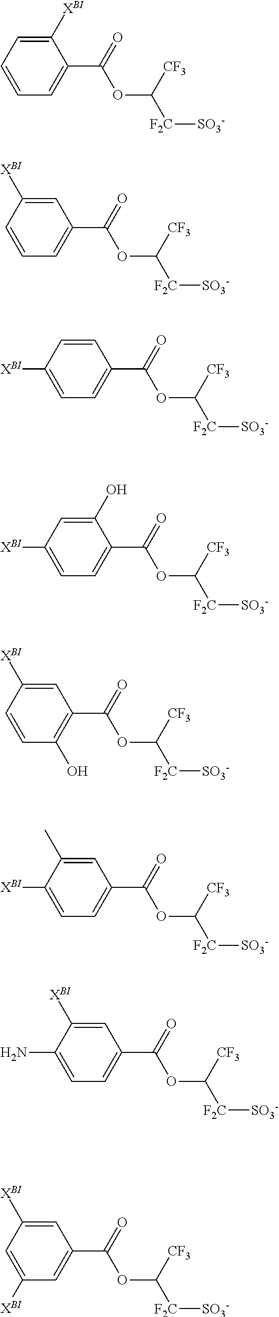

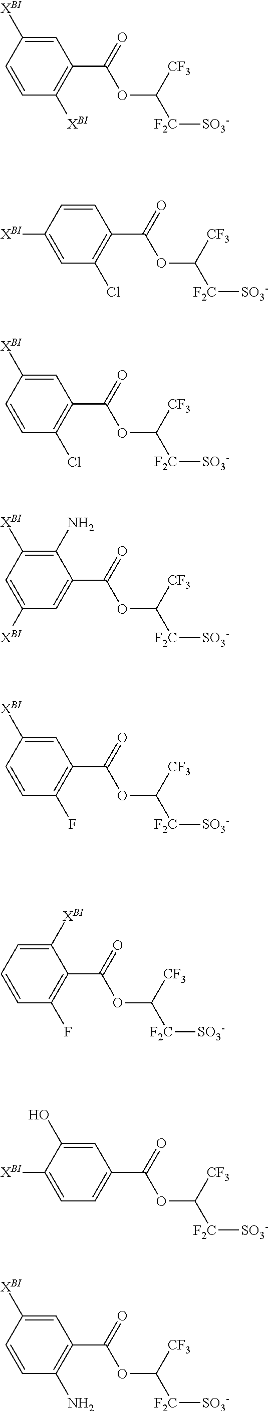

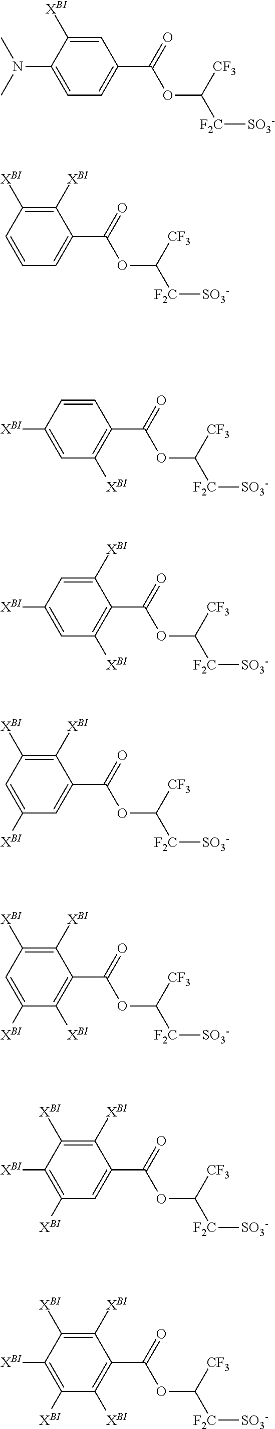

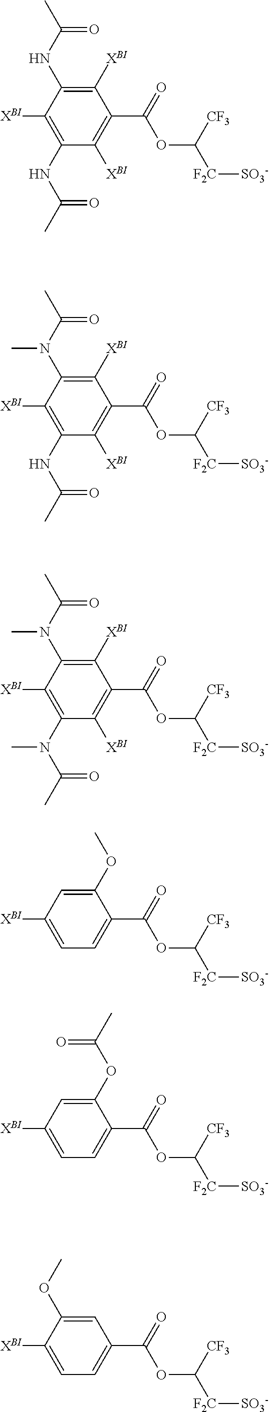

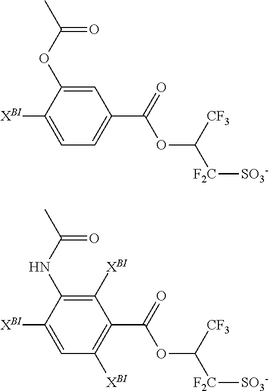

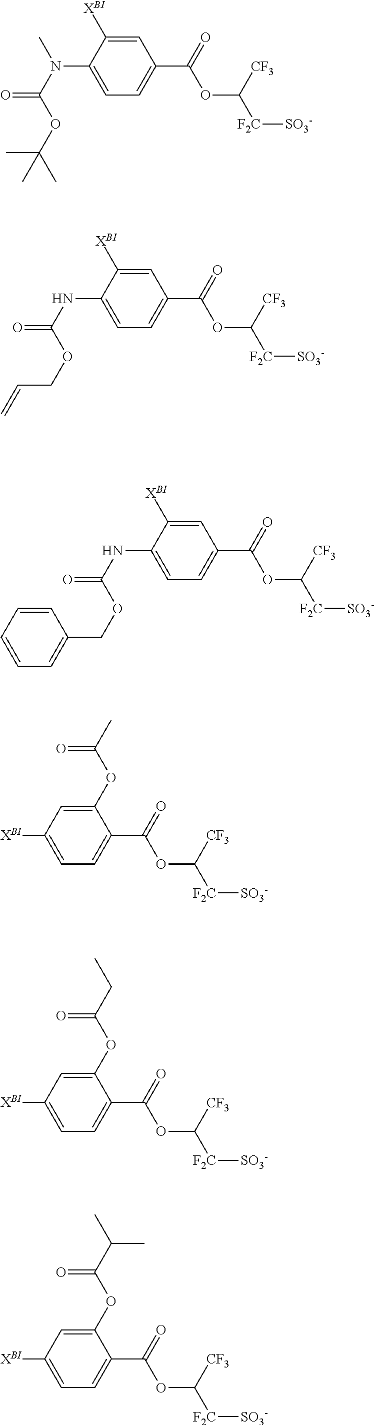

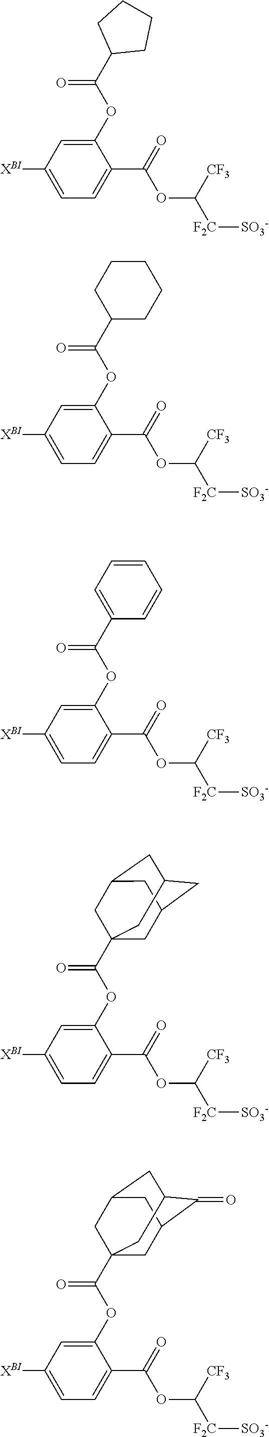

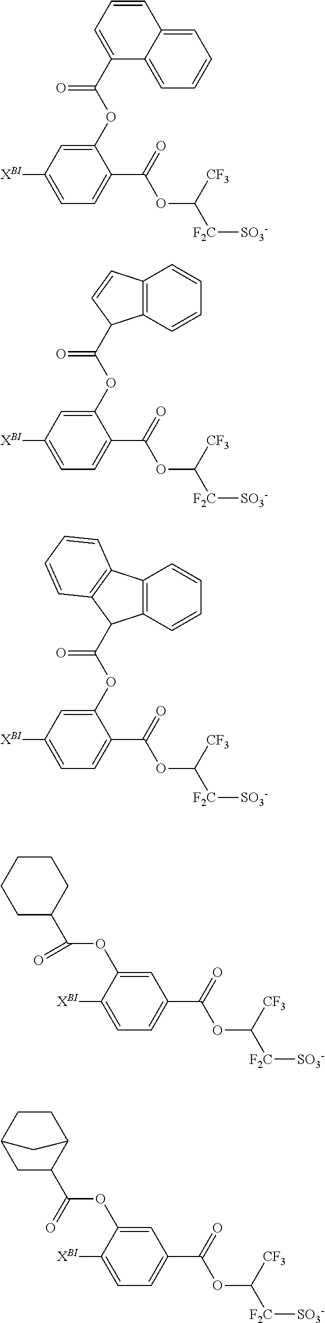

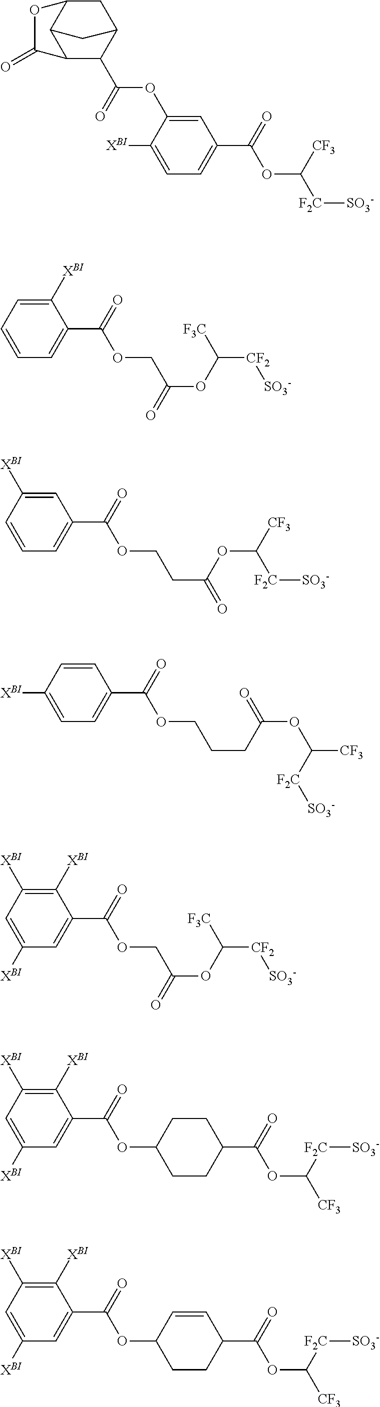

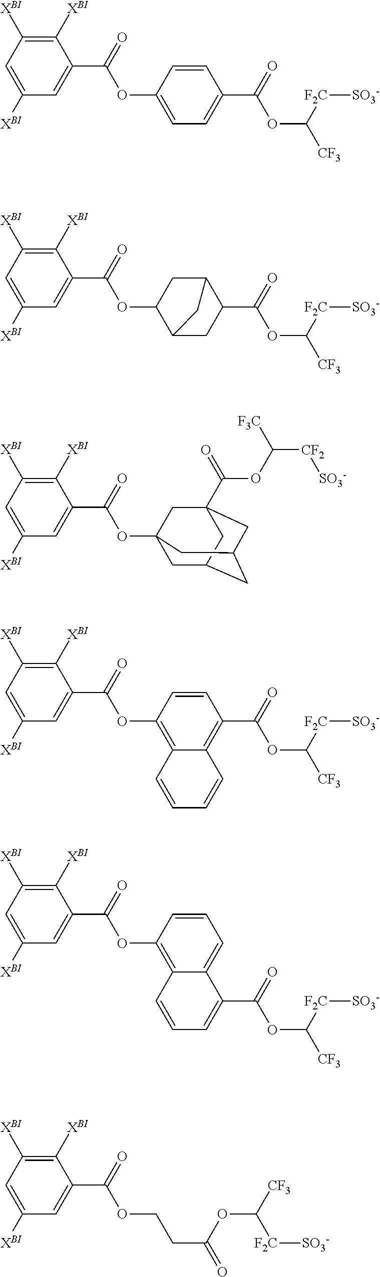

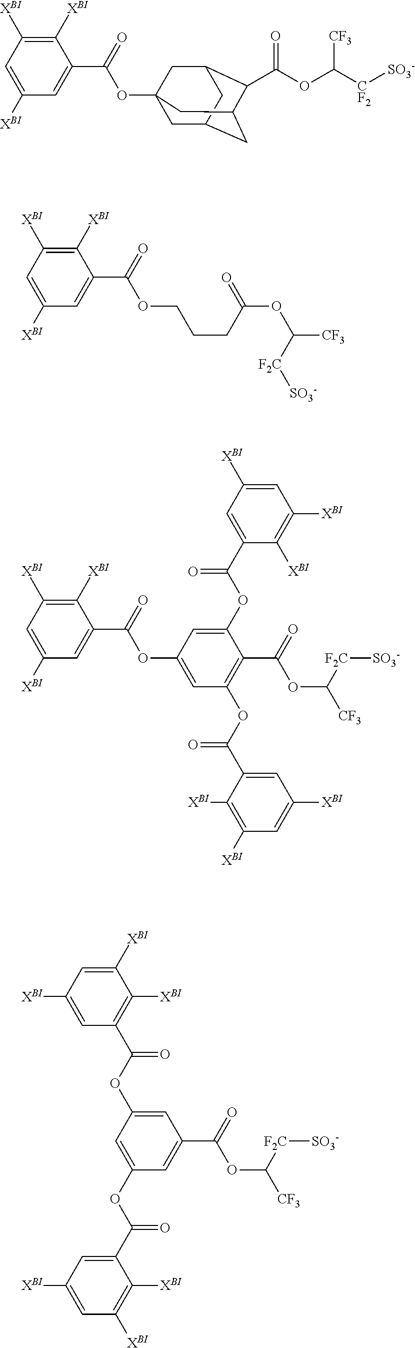

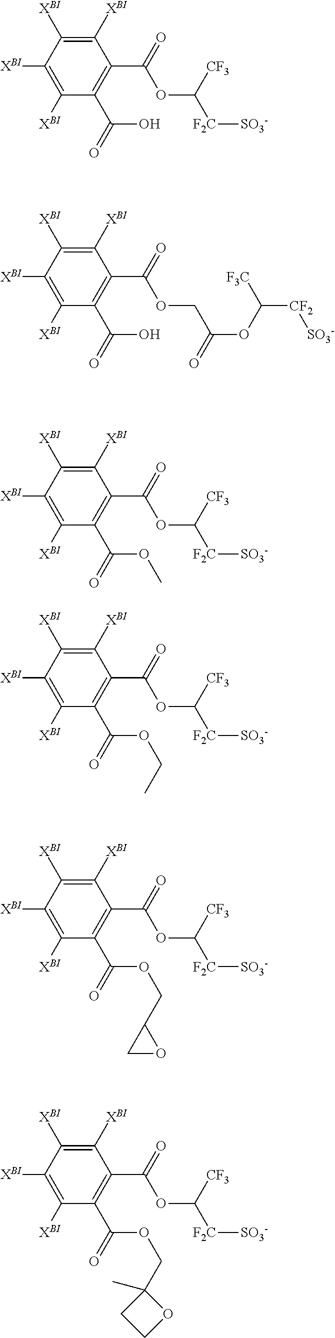

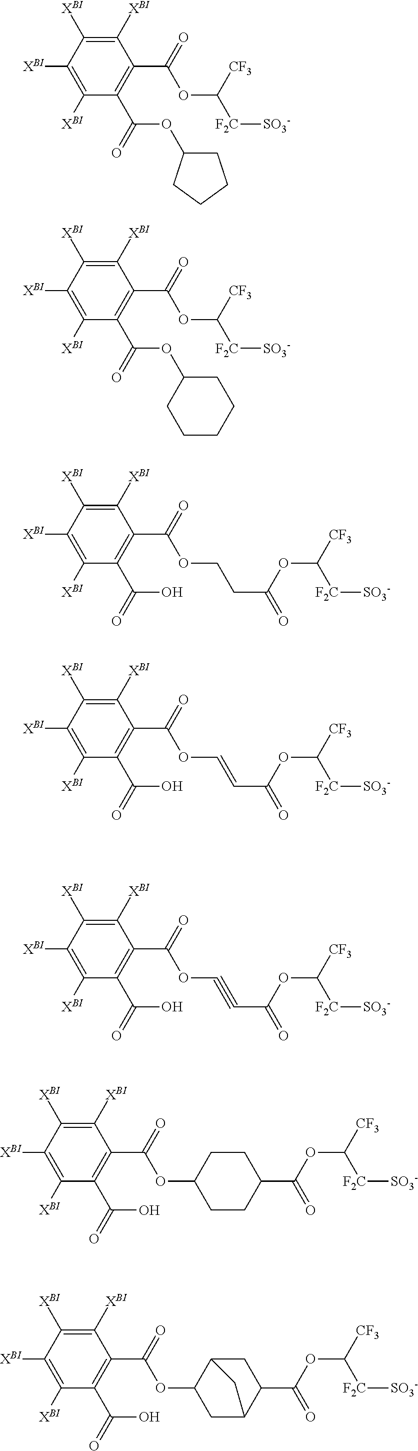

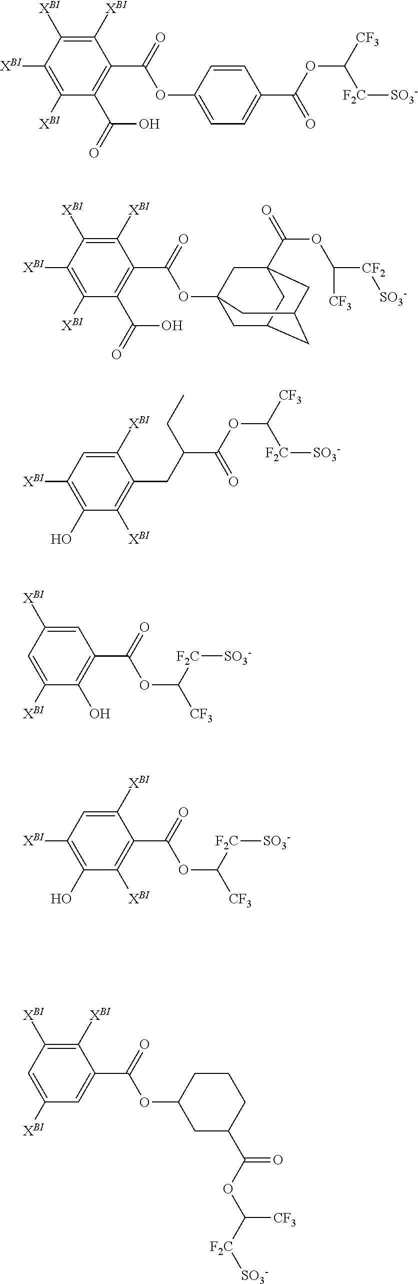

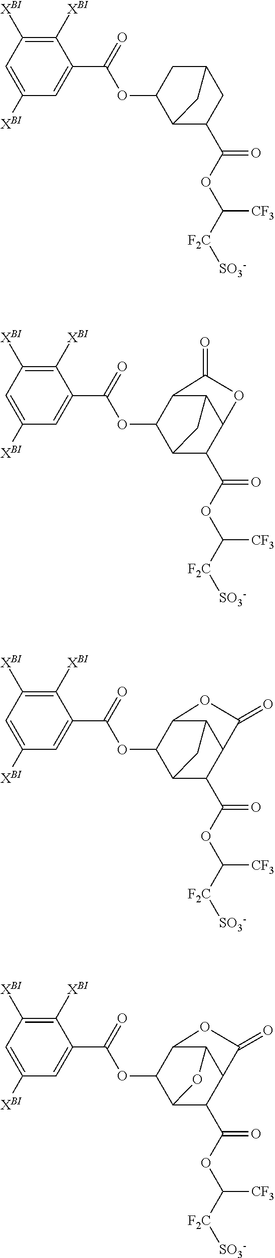

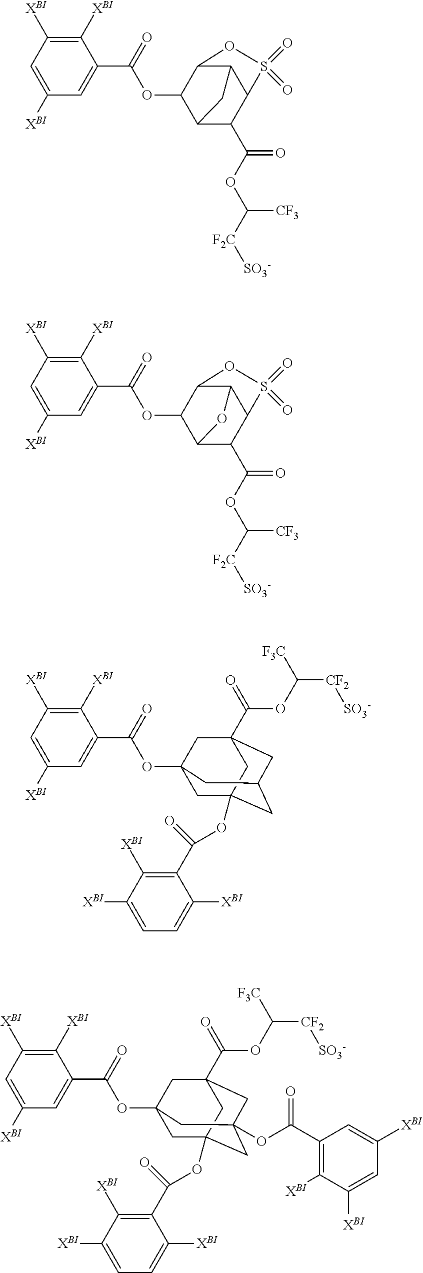

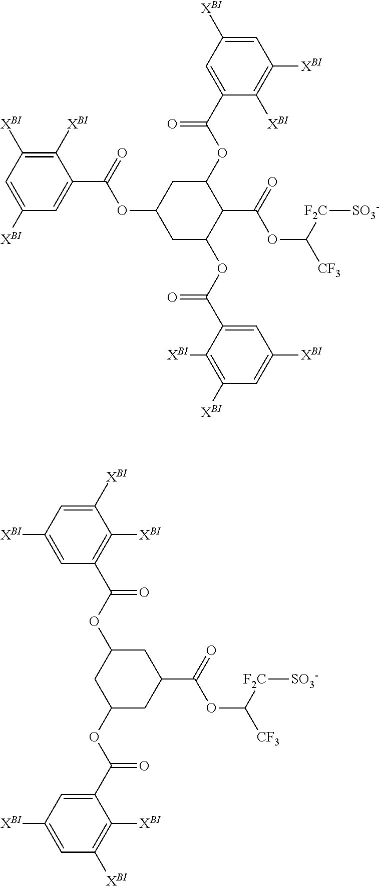

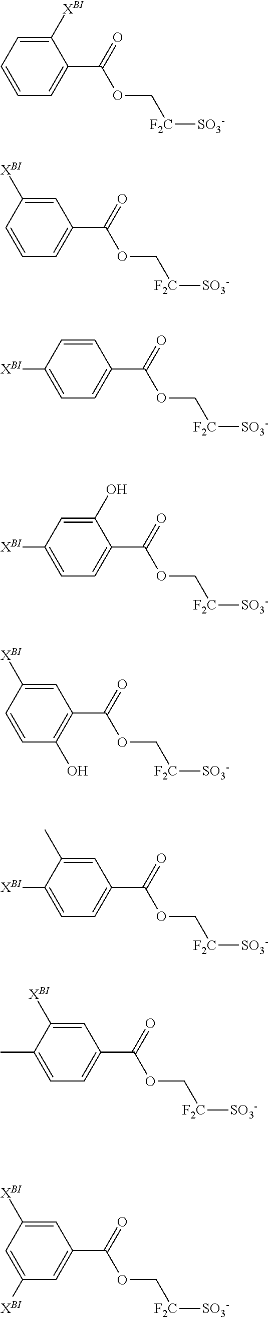

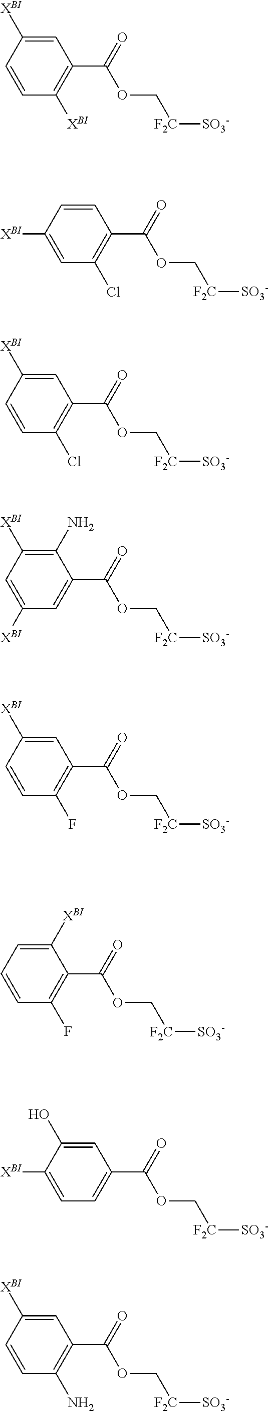

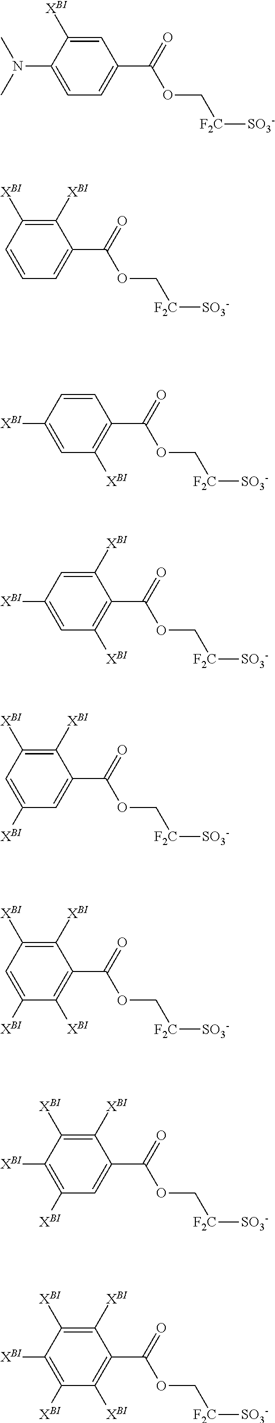

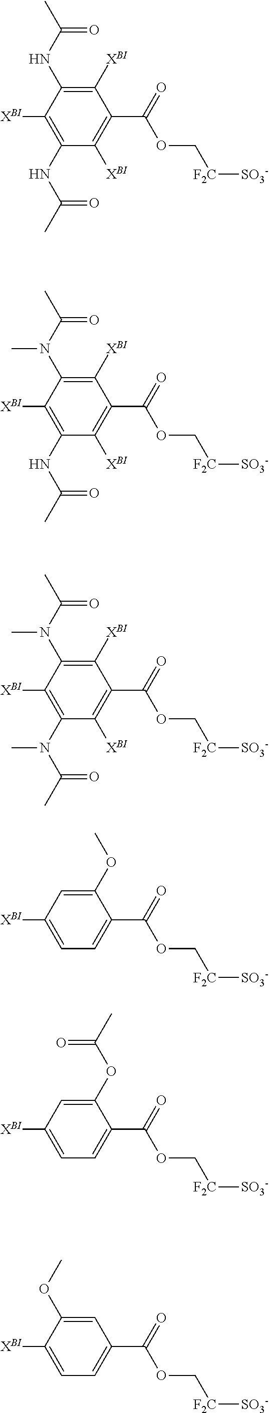

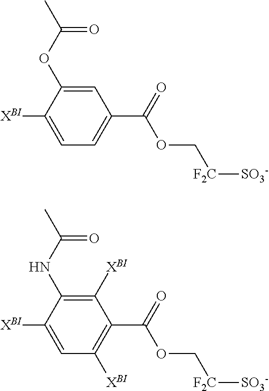

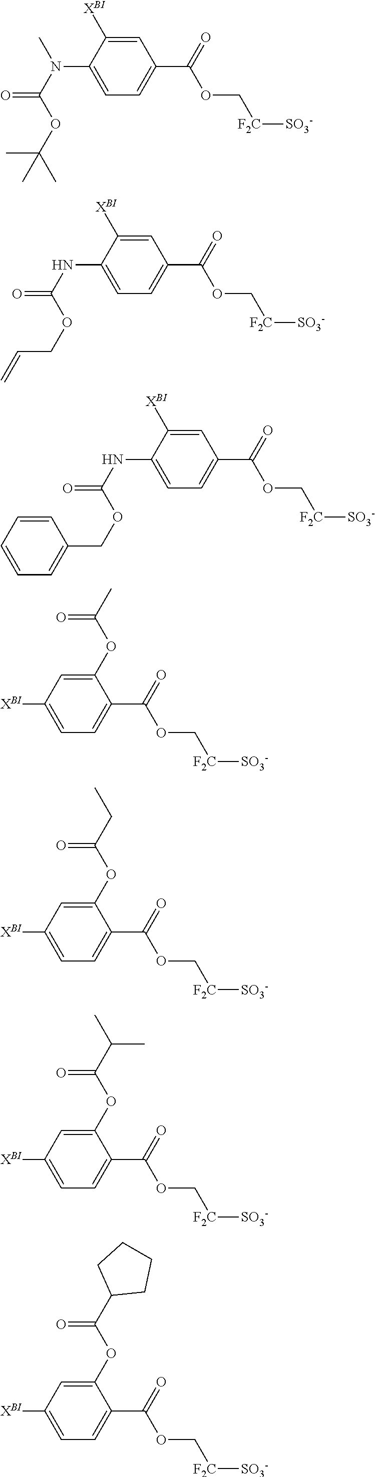

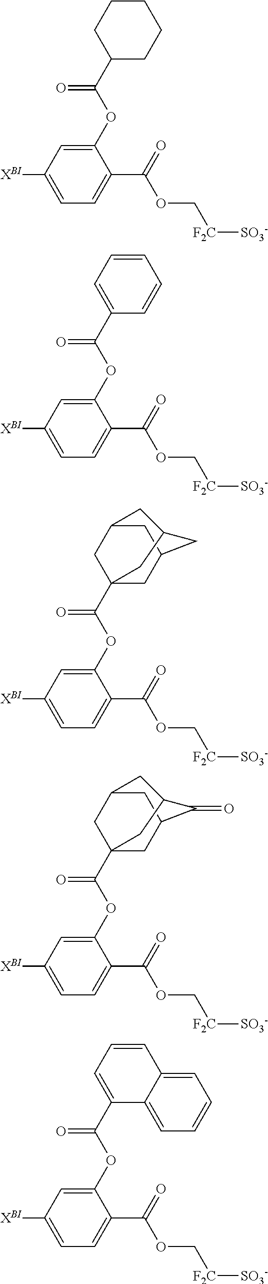

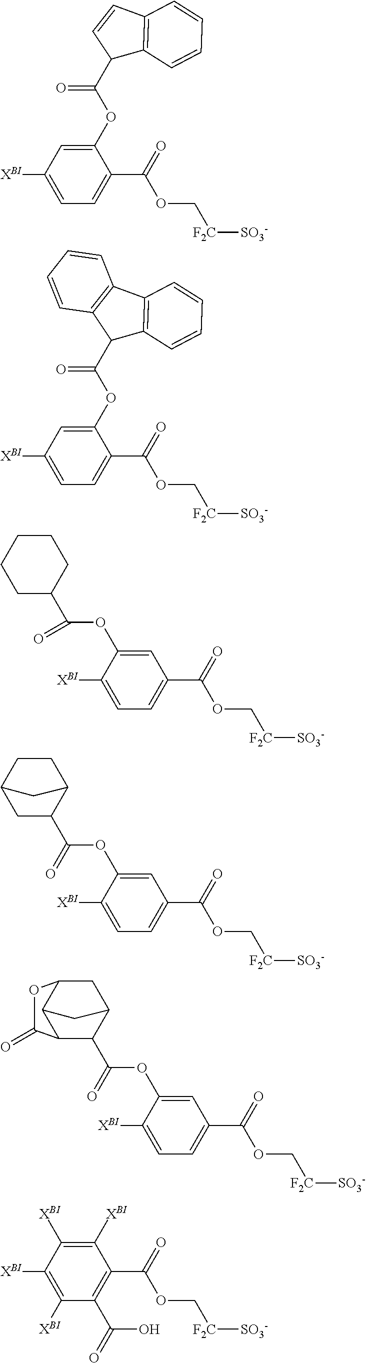

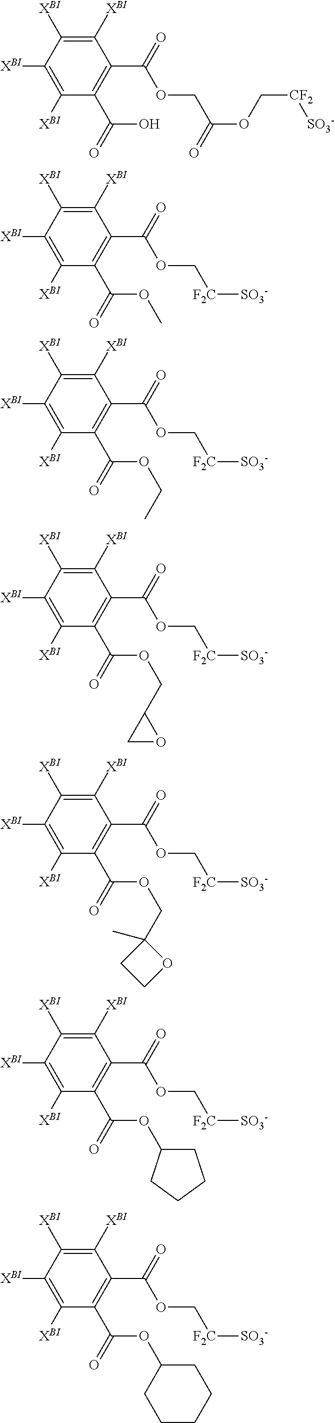

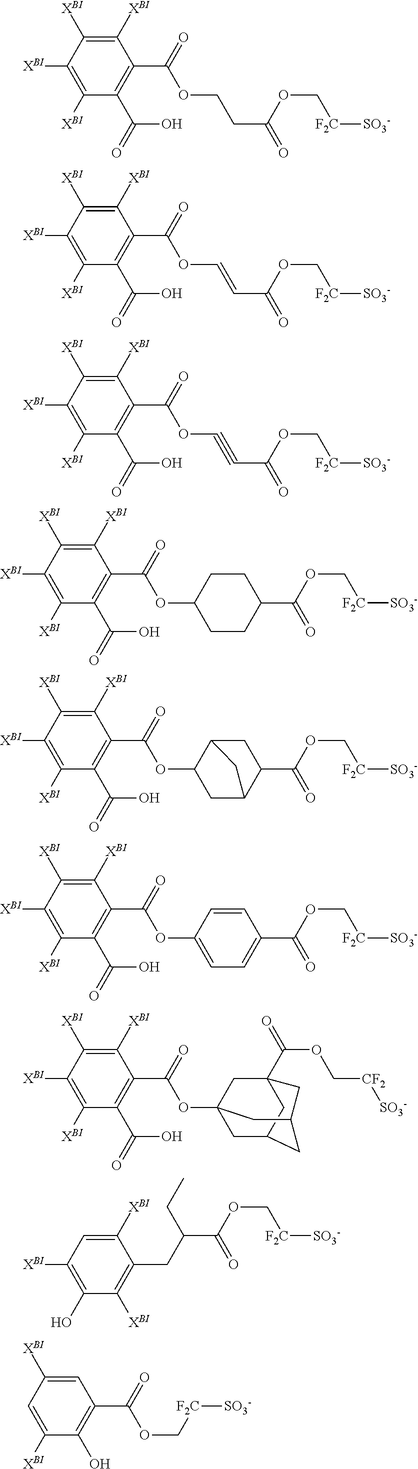

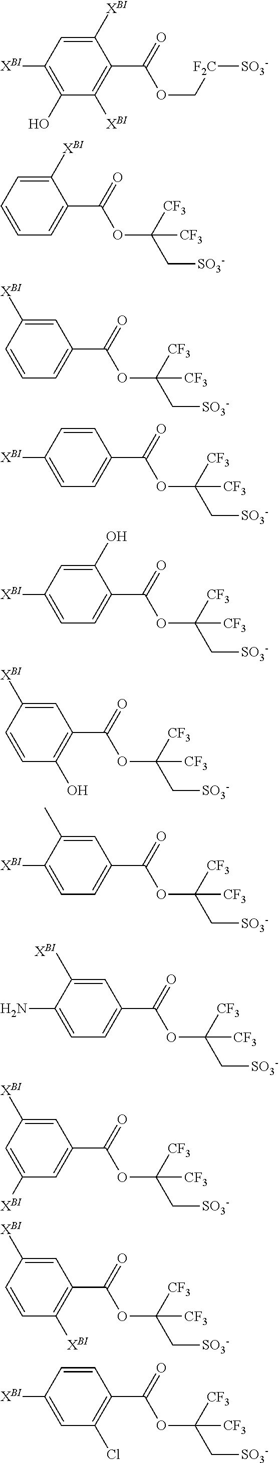

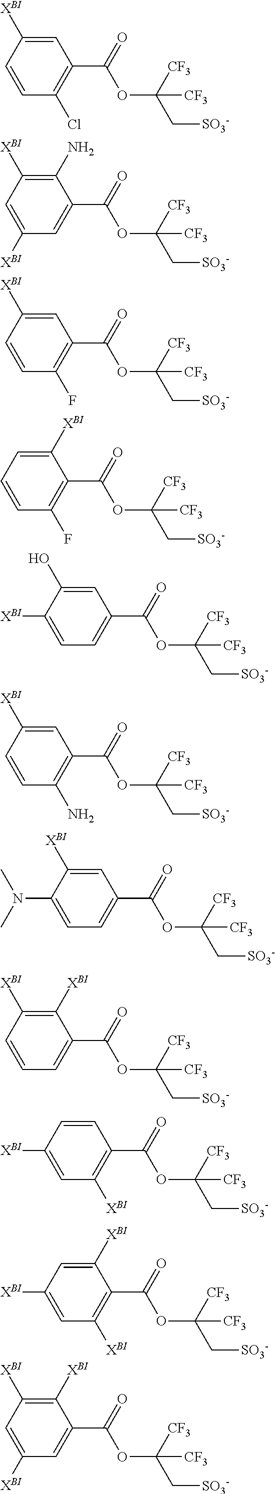

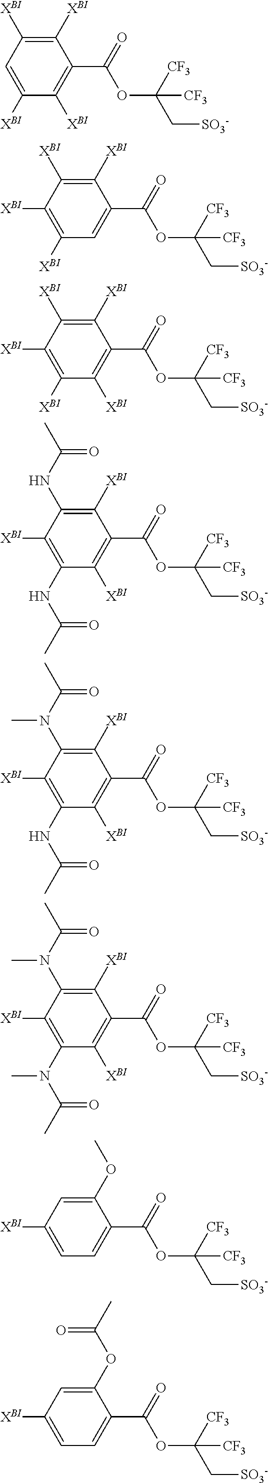

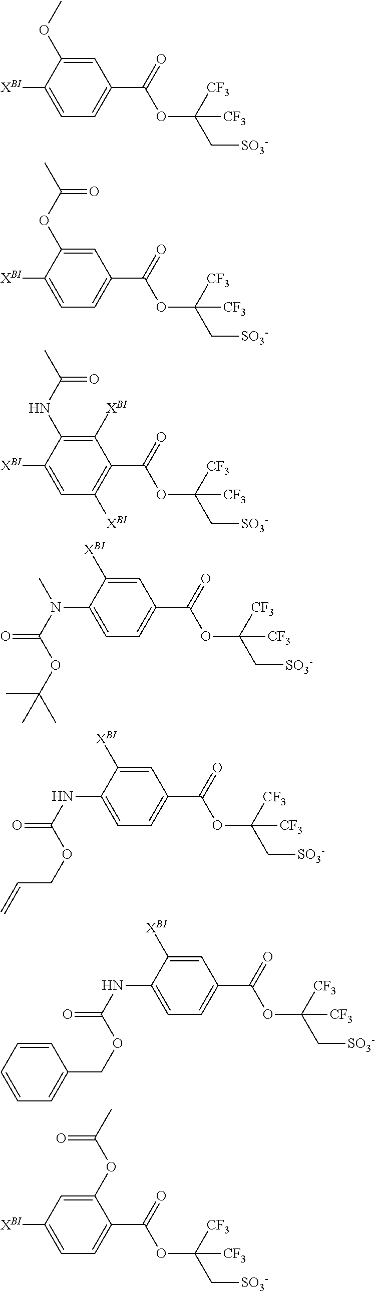

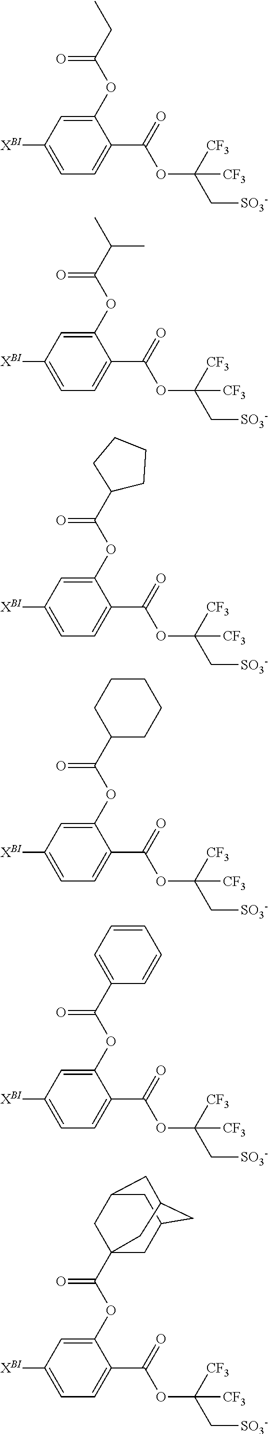

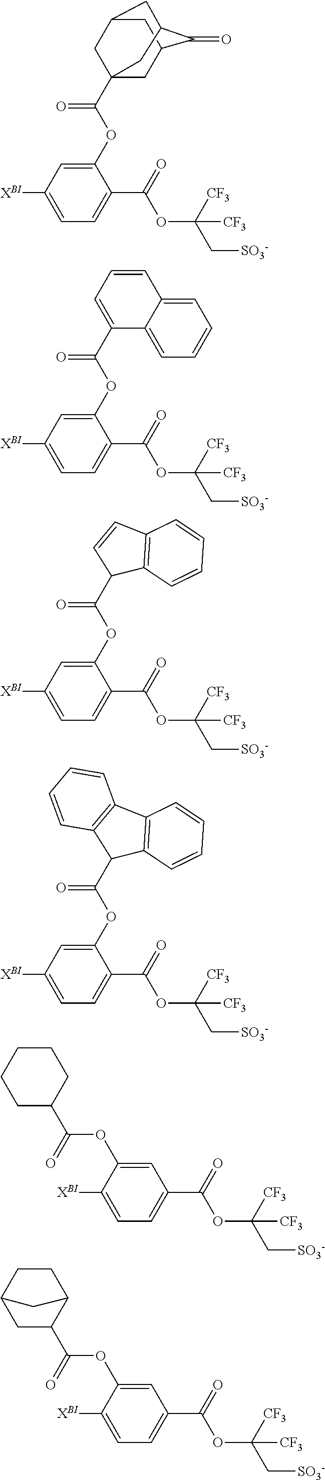

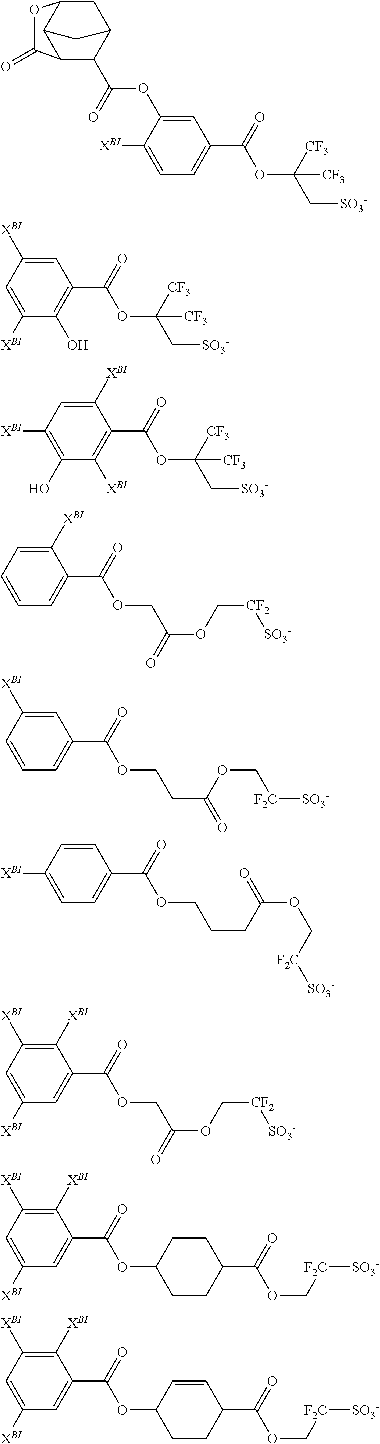

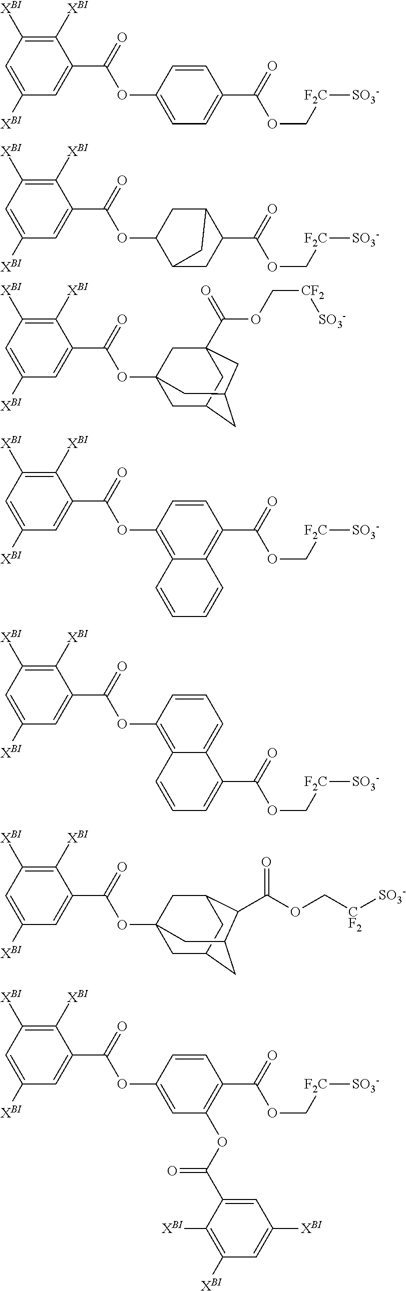

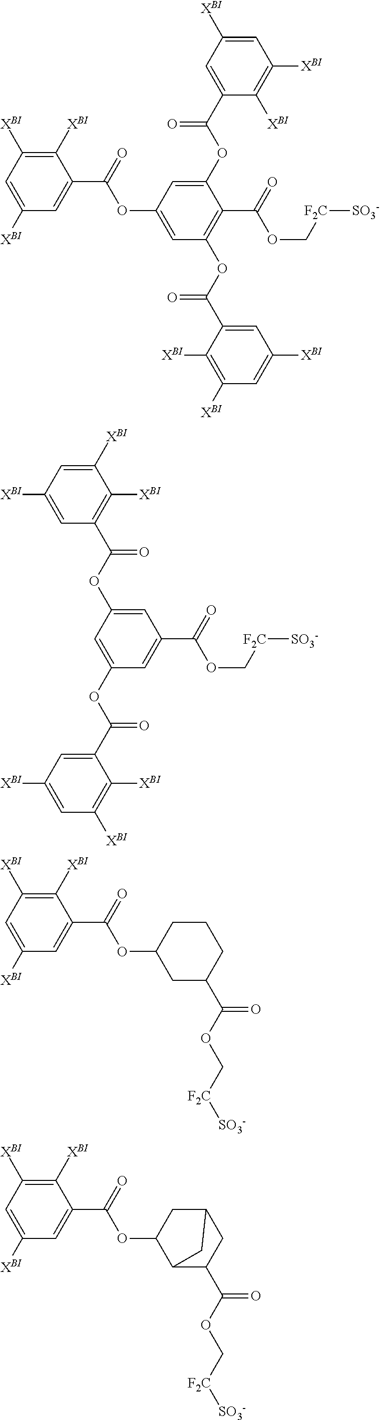

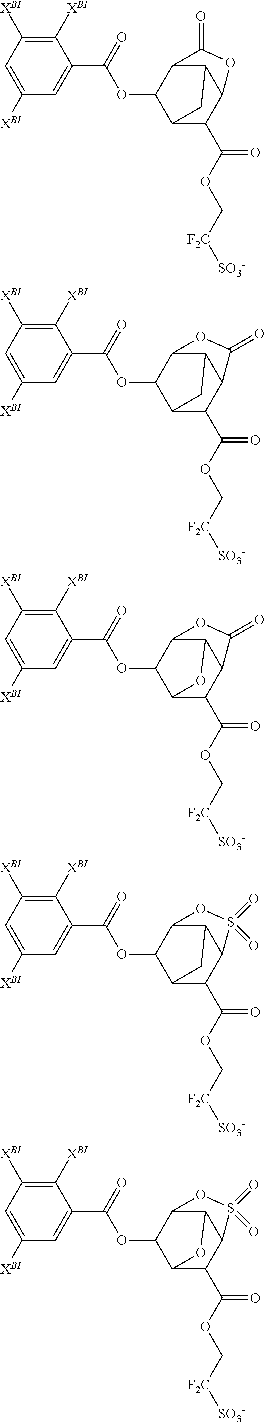

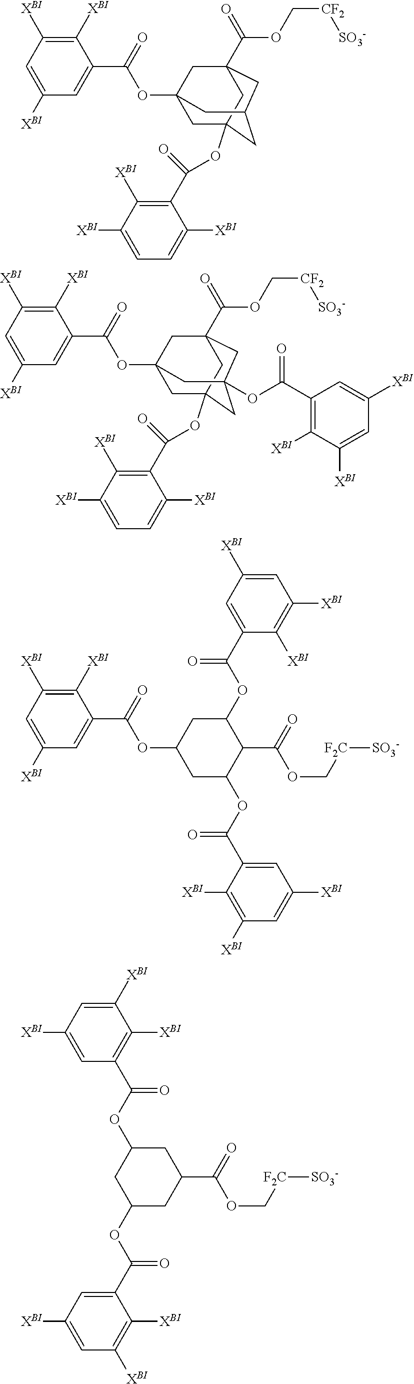

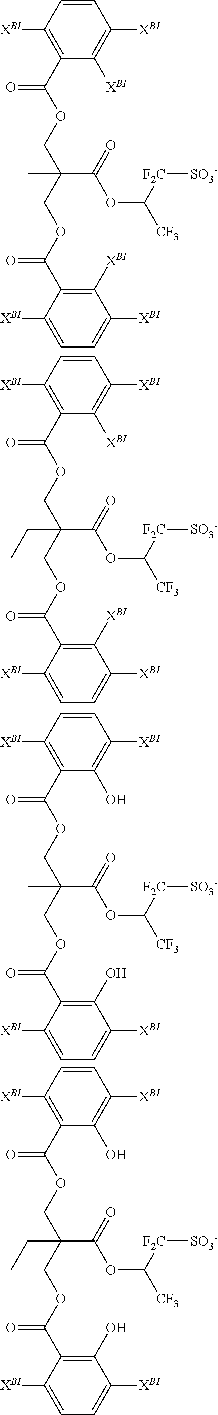

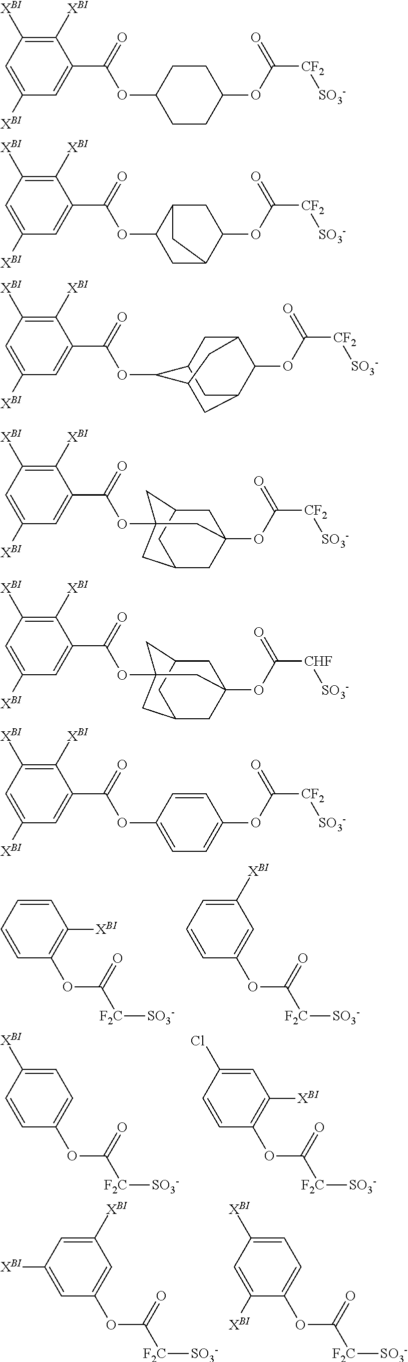

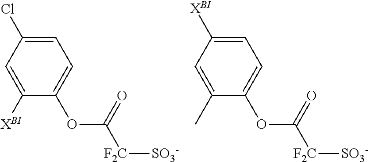

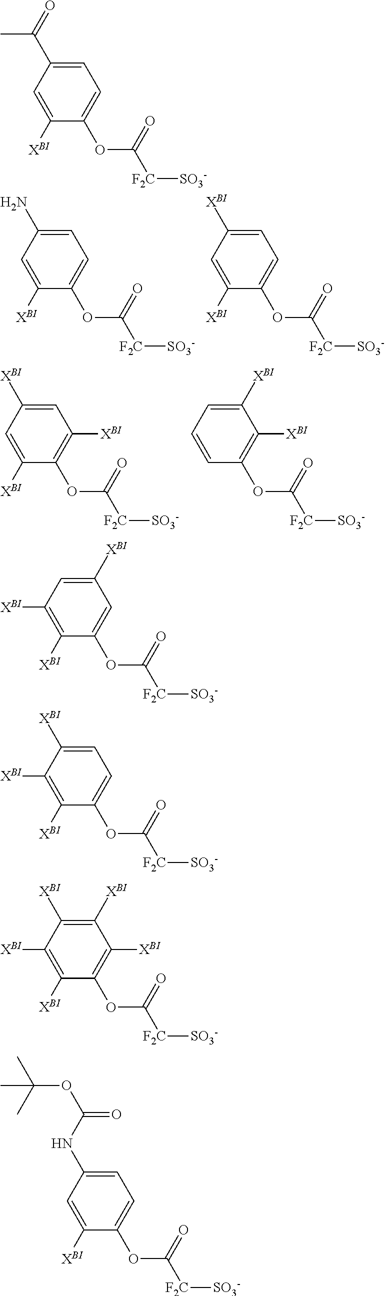

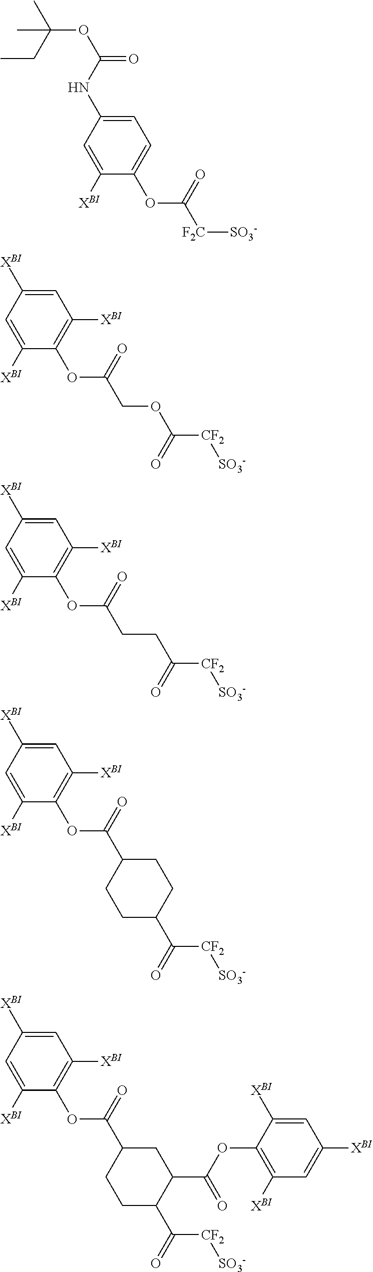

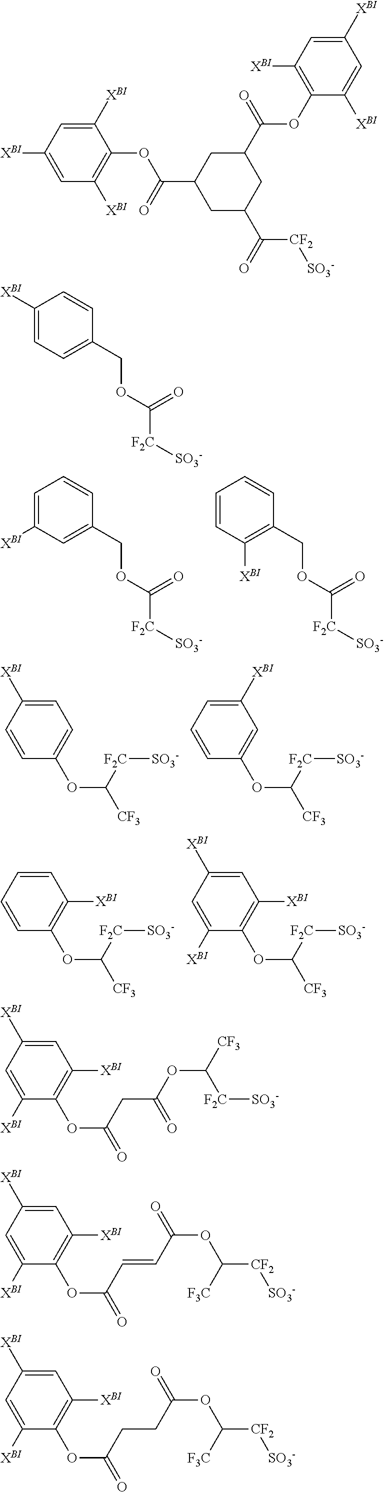

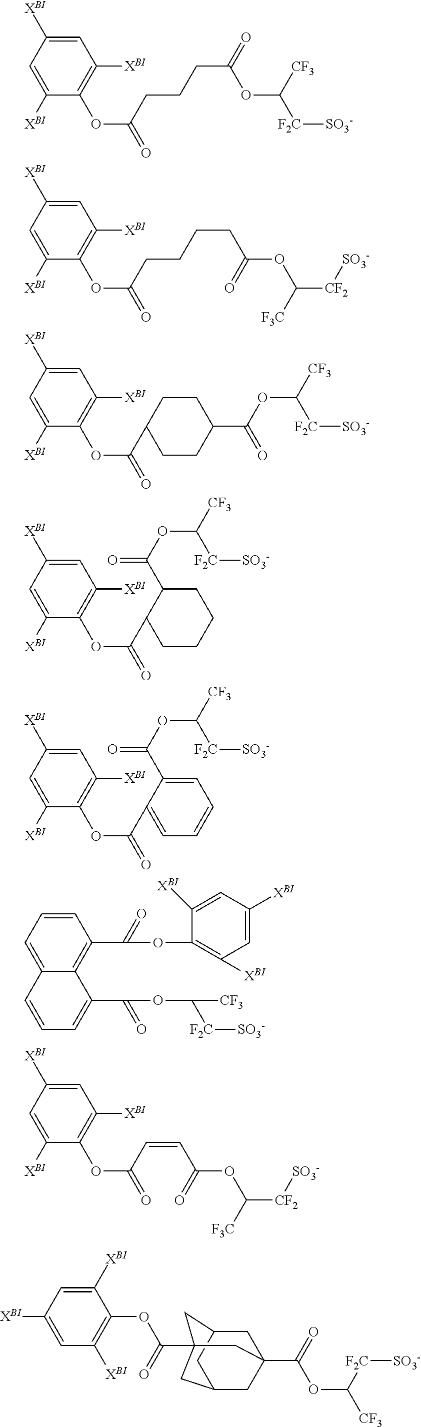

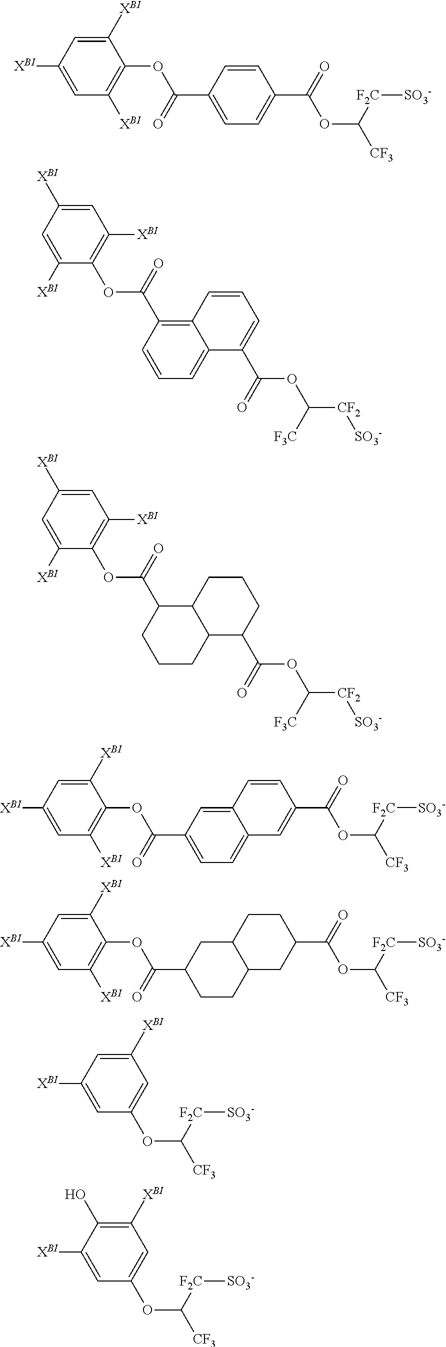

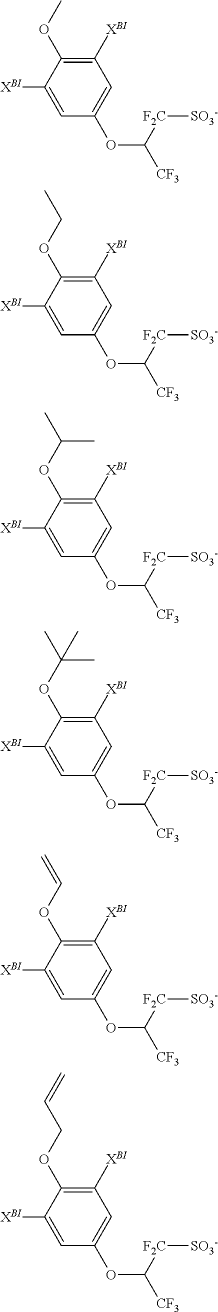

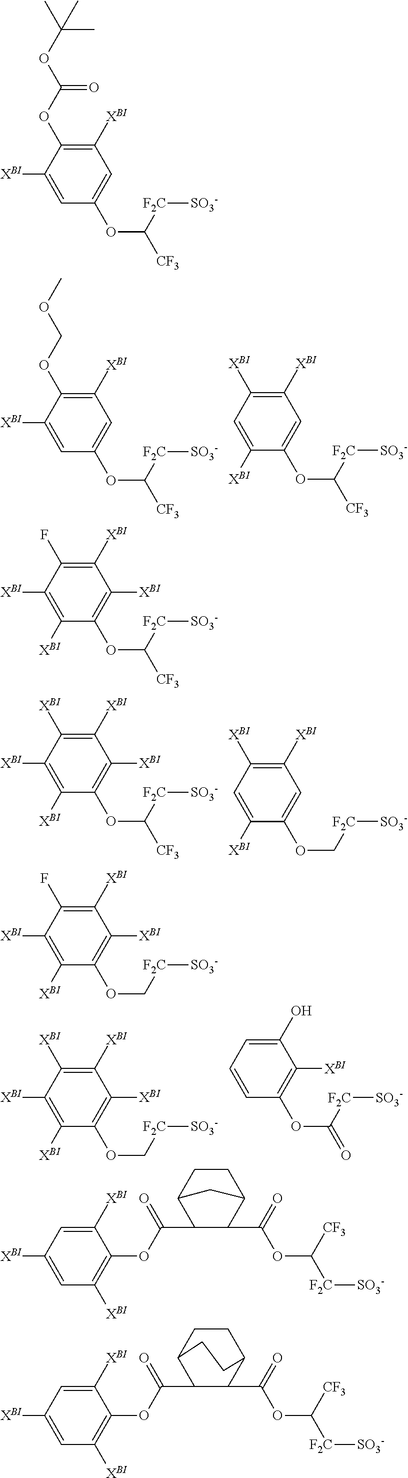

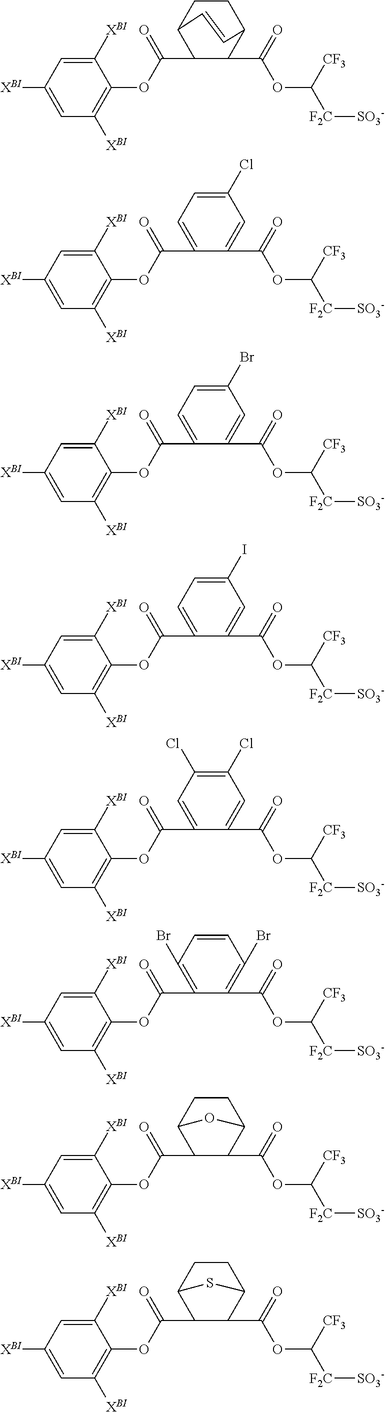

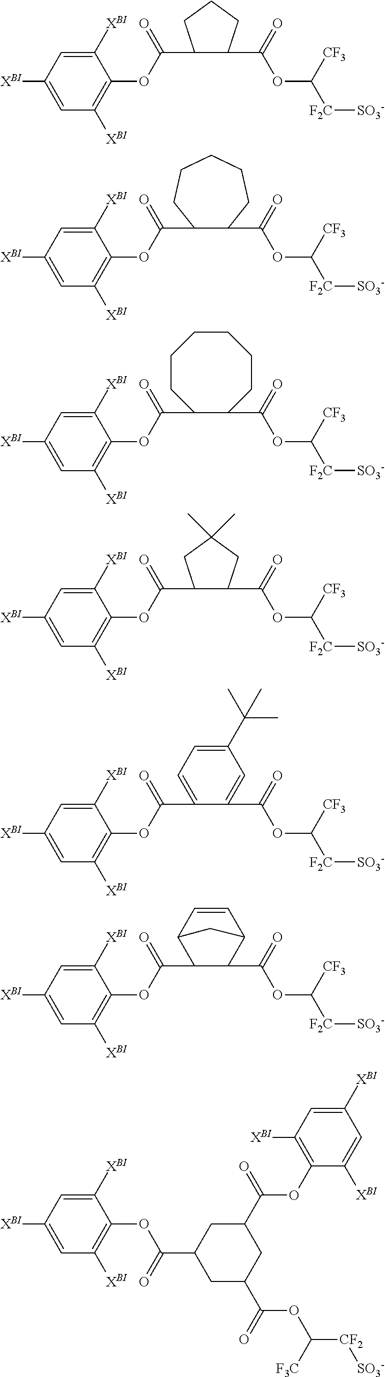

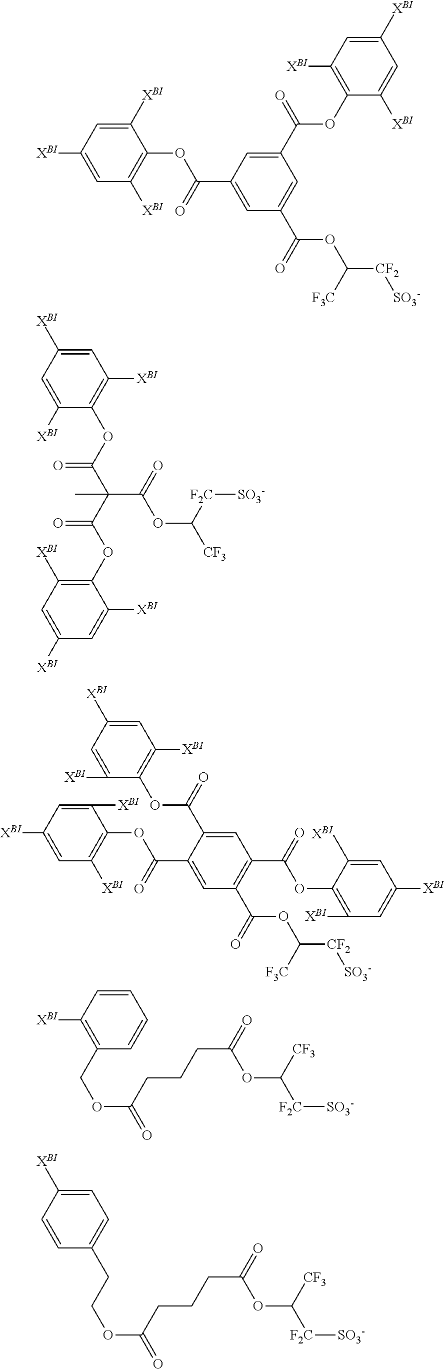

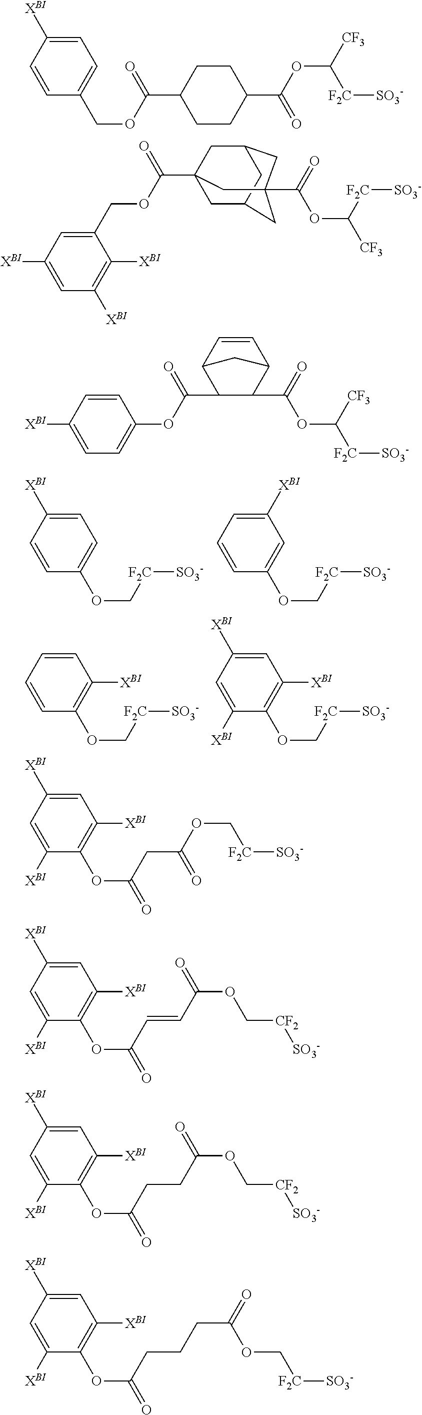

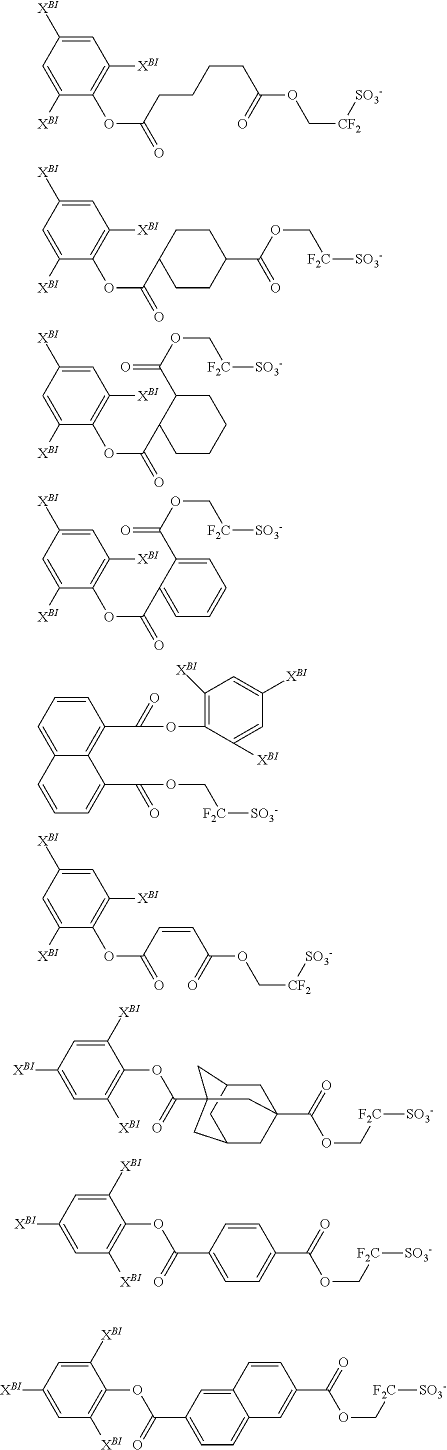

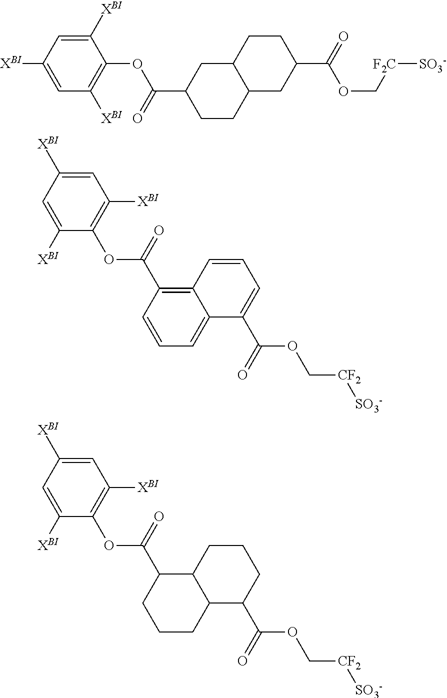

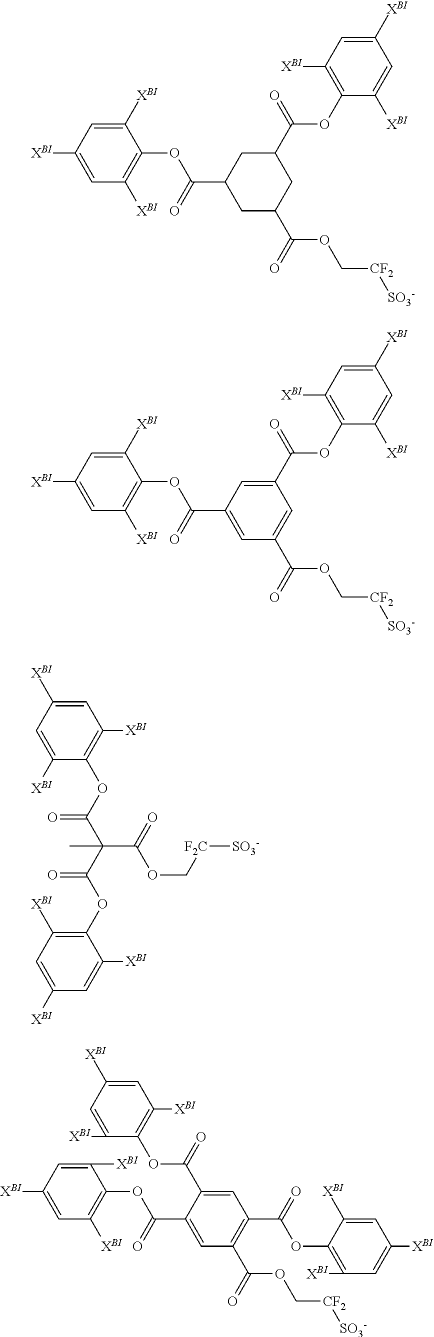

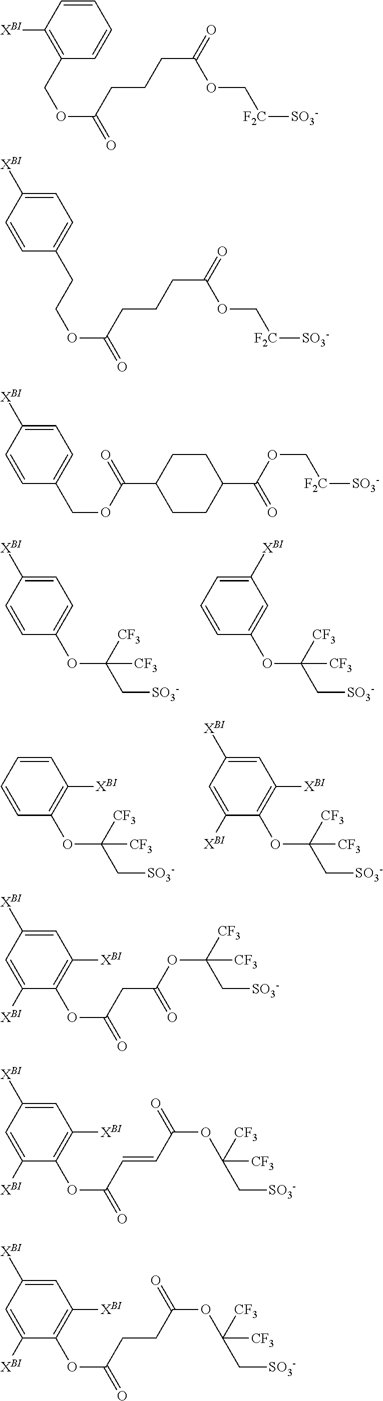

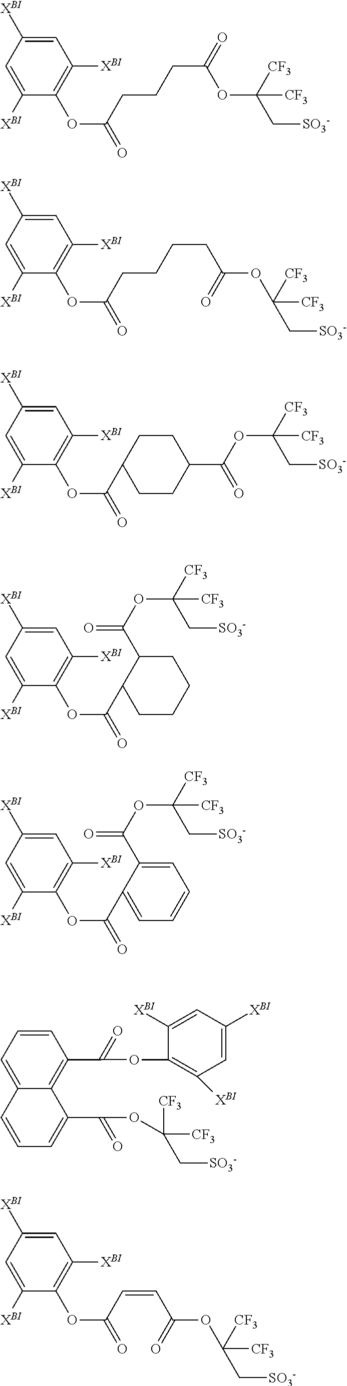

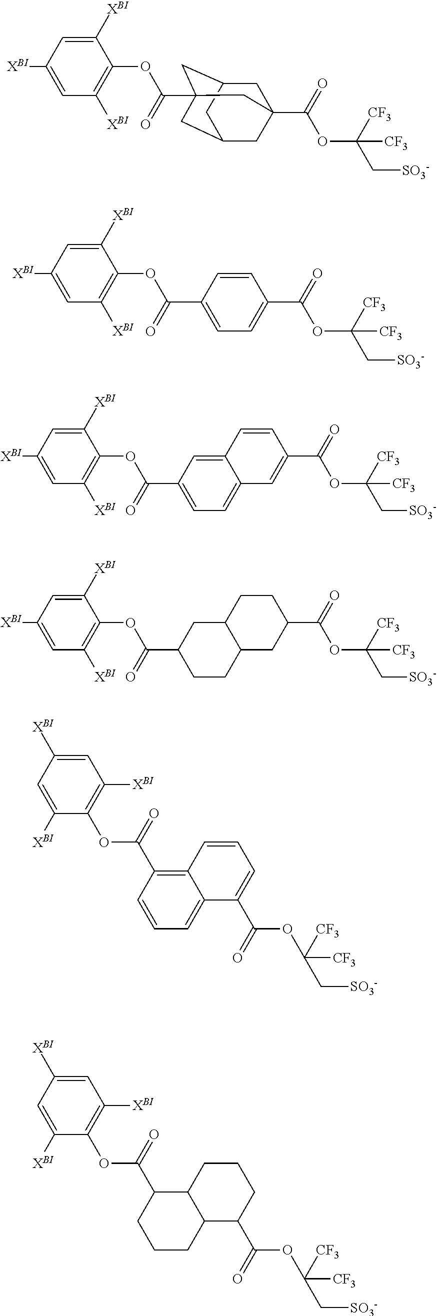

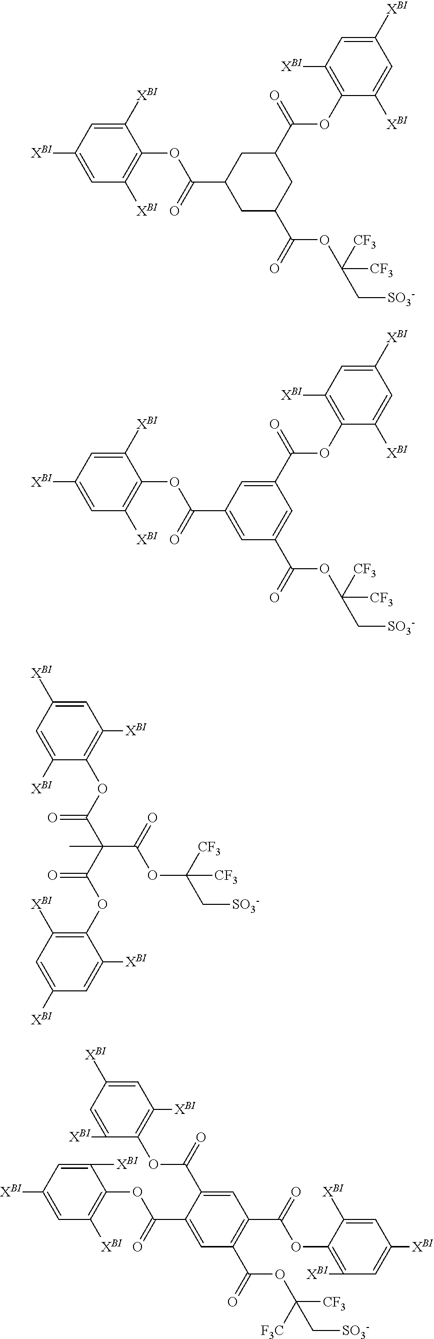

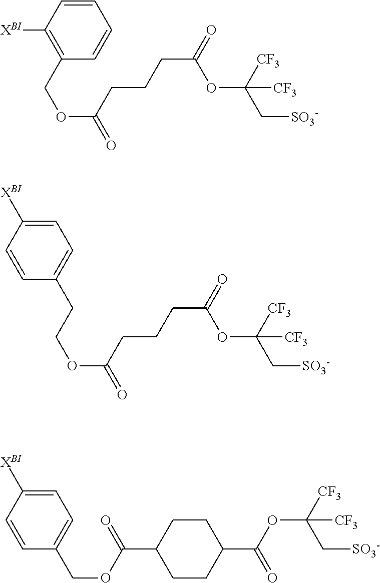

As the non-nucleophilic counterion, a fluorobenzenesulfonate anion bonded to an aromatic group containing an iodine atom as described in JP6648726B2, an anion having a mechanism of decomposition by an acid as described in WO2021/200056A1 and JP2021-070692A, an anion having a cyclic ether group as described in JP2018-180525A and JP2021-035935A, and an anion as described in JP2018-092159A can also be used.

As the non-nucleophilic counterion, further examples that may be used are anions of bulky benzenesulfonic acid derivatives that do not contain fluorine atoms as described in JP2006-276759A, JP2015-117200A, JP2016-065016A, and JP2019-202974A, and benzenesulfonic acid anions that do not contain fluorine atoms bonded to aromatic groups containing iodine atoms and alkylsulfonic acid anions as described in JP6645464B2.

As the non-nucleophilic counterion, further examples that may be used include an anion of a bissulfonic acid as described in JP2015-206932A, an anion of sulfonamide or sulfonimide having a sulfonic acid on one side and a different group on the other side as described in WO2020/158366A1, and an anion having a sulfonic acid on one side and a carboxylic acid on the other side as described in JP2015-024989A can be used.

In the general formulae (c2) and (c3), “dl” and “d2” each independently represent an integer from 0 to 3, and are preferably 1.

In the general formula (c4), “e1” is 0 or 1. “e2” is an integer from 0 to 4. “e3” is an integer from 0 to 4. Provided that when “e1” is 0, 0≤e2+e3≤4, and when “e1” is 1, 0≤e2+e3≤6.

In the general formulae (c2), (c3), and (c4), L¹ is a single bond, an ether bond, an ester bond, a carbonyl group, a sulfonate ester bond, a sulfonamide bond, a carbonate bond, or a carbamate bond. Among these, from the viewpoint of synthesis, an ether bond, an ester bond, and a carbonyl group are preferable, and an ester bond and a carbonyl group are more preferable.

In the general formula (c2), Rf¹ and Rf² are each independently a fluorine atom, or a fluorinated saturated hydrocarbyl group having 1 to 6 carbon atoms. Of these, Rf¹ and Rf² are preferably both fluorine atoms, in order to increase the acid strength of the generated acid. Rf³ and Rf⁴ are each independently a hydrogen atom, a fluorine atom, or a fluorinated saturated hydrocarbyl group having 1 to 6 carbon atoms. Of these, at least one of Rf³ and Rf⁴ is preferably a trifluoromethyl group, in order to improve solvent solubility.

In the general formula (c3), Rf⁵ and Rf⁶ are each independently a hydrogen atom, a fluorine atom, or a fluorinated saturated hydrocarbyl group having 1 to 6 carbon atoms. Provided that all Rf⁵ and Rf⁶ are not simultaneously hydrogen atoms. Of these, at least one of Rf⁵ and Rf⁶ is preferably a trifluoromethyl group, in order to improve solvent solubility.

In the general formula (c4), Rf⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms. Rf⁷ is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. When “f” is 2, 3, or 4, each Rf⁷ may be the same as or different from each other.

In the general formula (c4), R³³ is a halogen atom other than fluorine atom, or a hydrocarbyl group having 1 to 20 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R¹ in the general formula (1) but are not limited thereto. In addition, when “e3” is 2, 3, or 4, each R³³ may be the same as or different from each other.

In addition, when “e3” is 2, 3, or 4, a plurality of R³³ may be bonded to each other to form a ring together with the carbon atoms to which they are bonded. Specific examples of the ring formed in this case include a cyclopropane ring, a cyclobutane ring, a cyclopentane ring, a cyclohexane ring, a norbornane ring, and an adamantane ring. In addition, some or all of the hydrogen atoms in the ring may be substituted with groups containing heteroatoms such as an oxygen atom, a sulfur atom, a nitrogen atom, and a halogen atom, and some of the —CH₂— may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the anion of the repeating unit-c2 include, but are not limited to, those shown below. Note that, in the following formulae, R^{A} is as defined above, and Me is a methyl group.

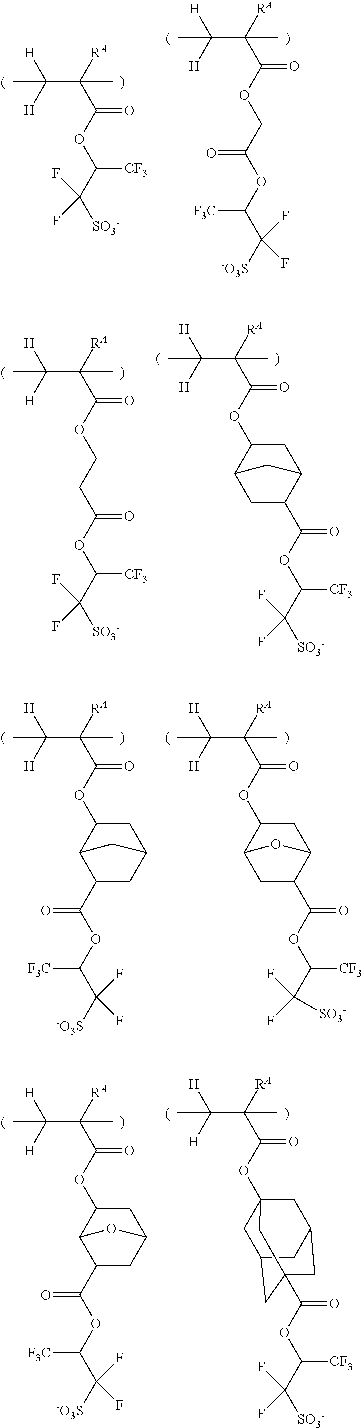

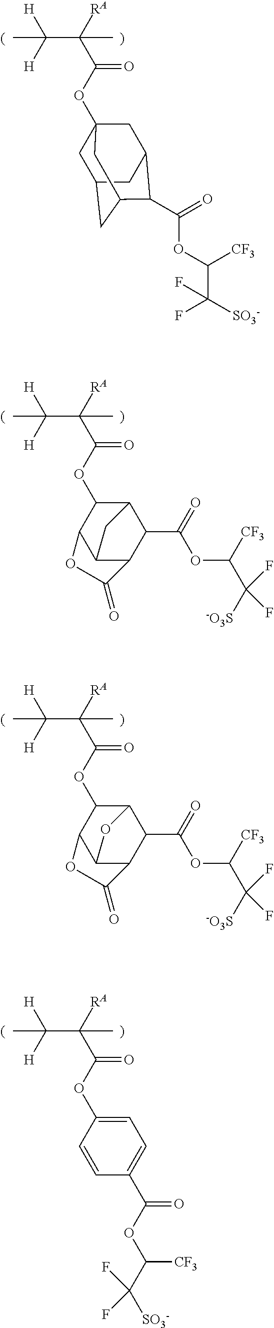

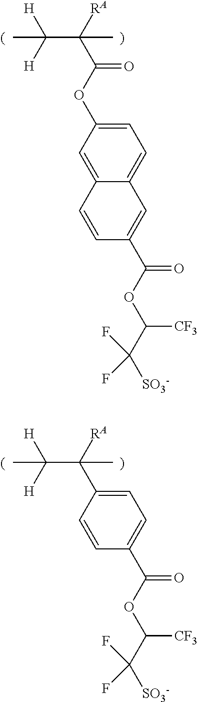

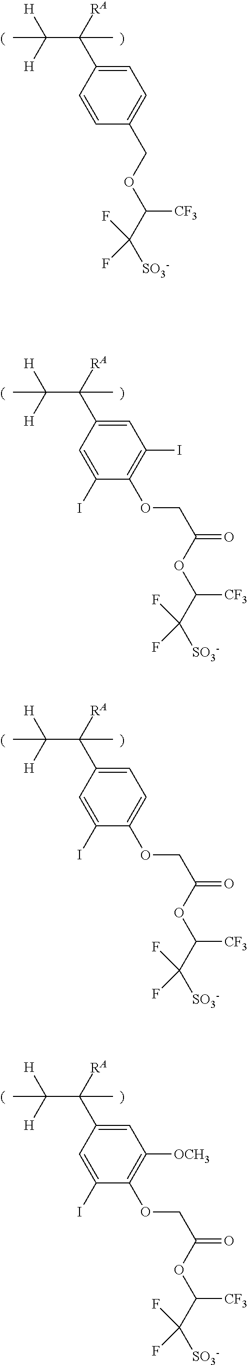

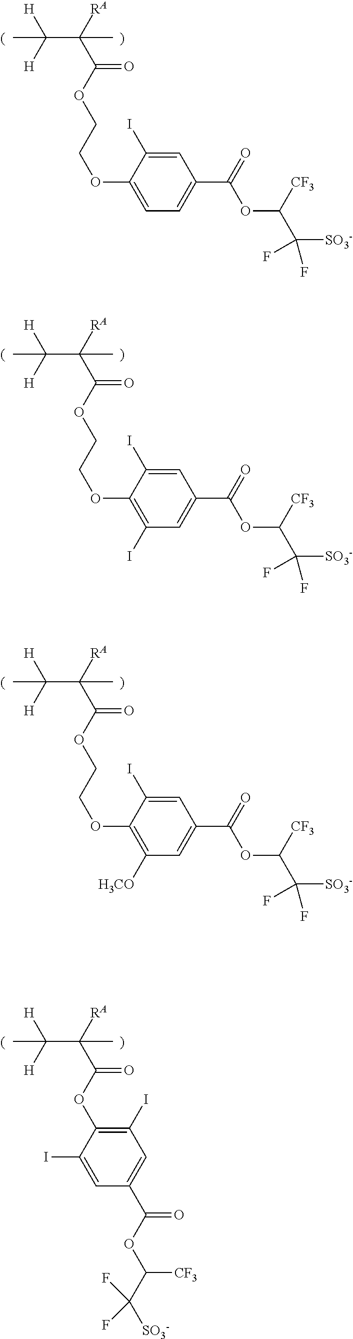

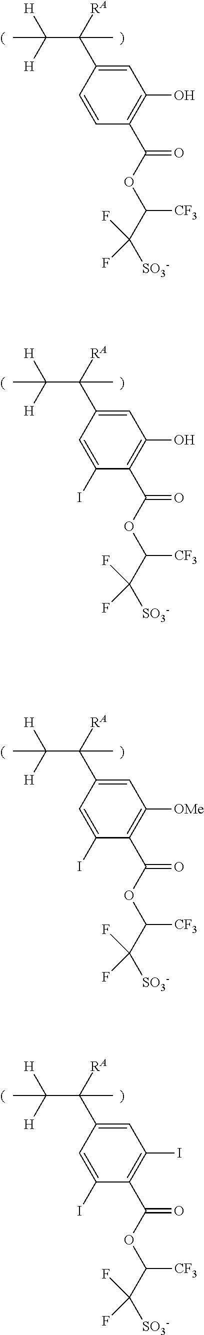

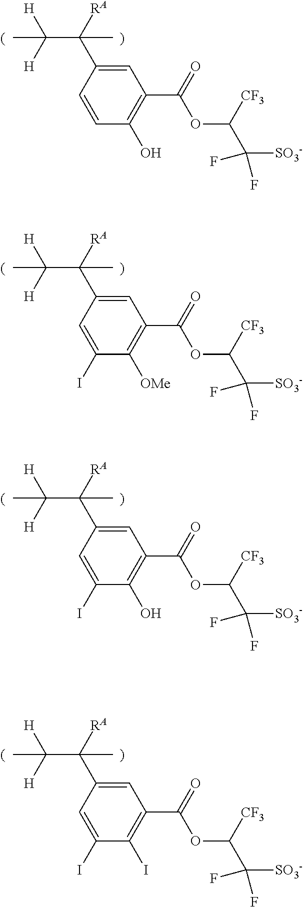

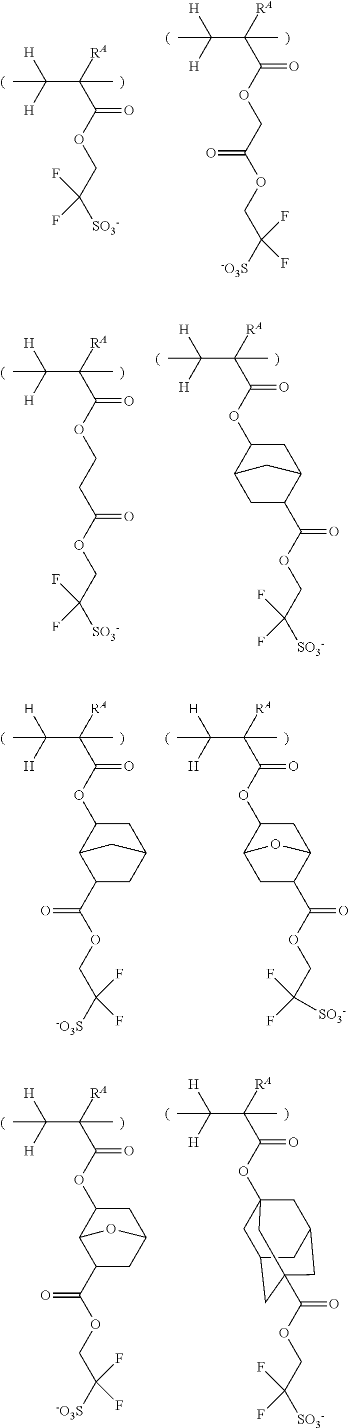

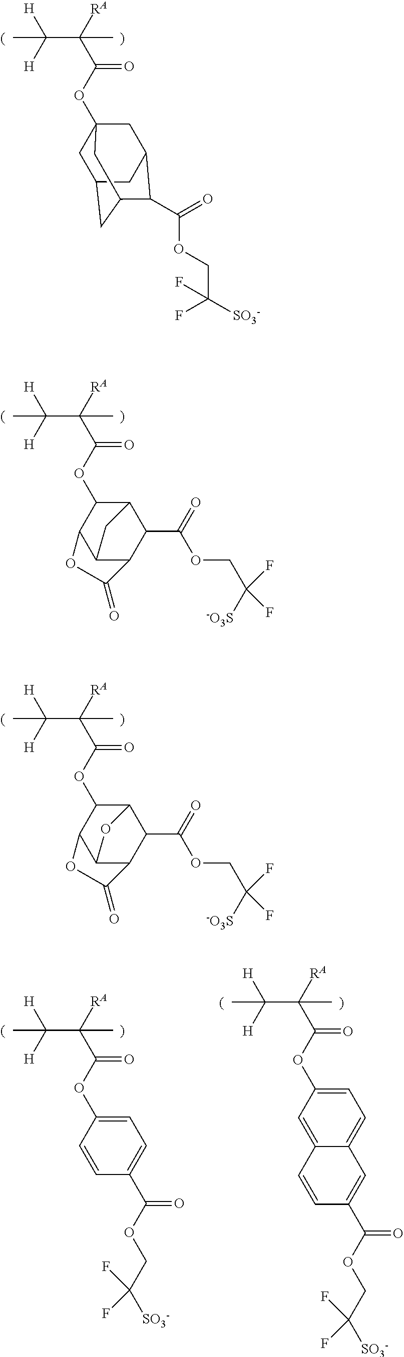

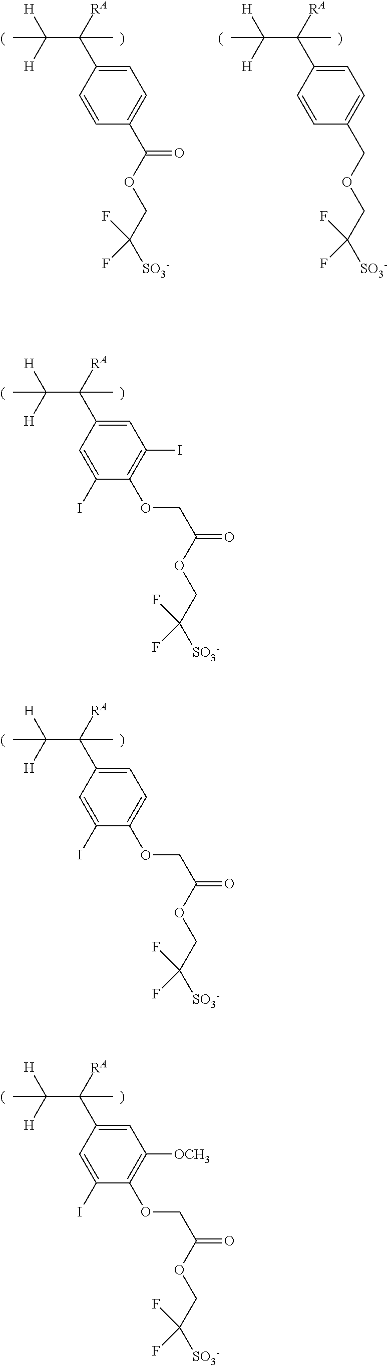

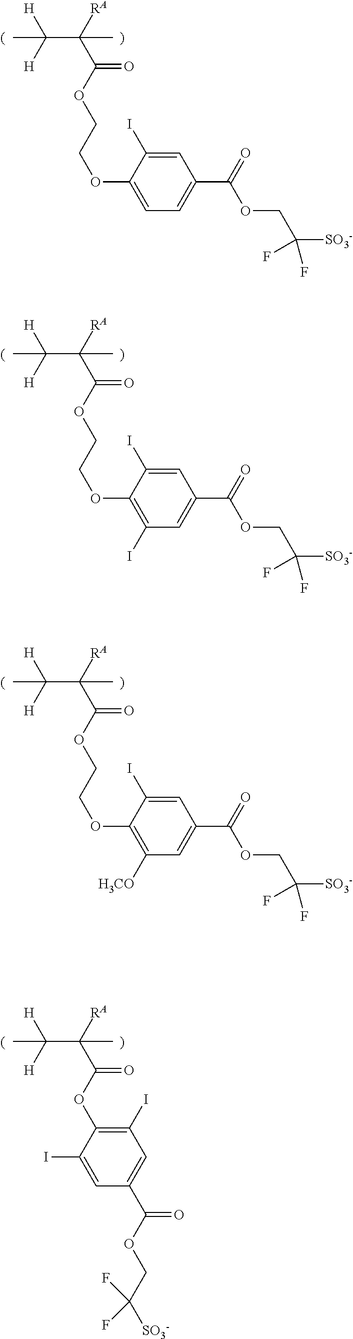

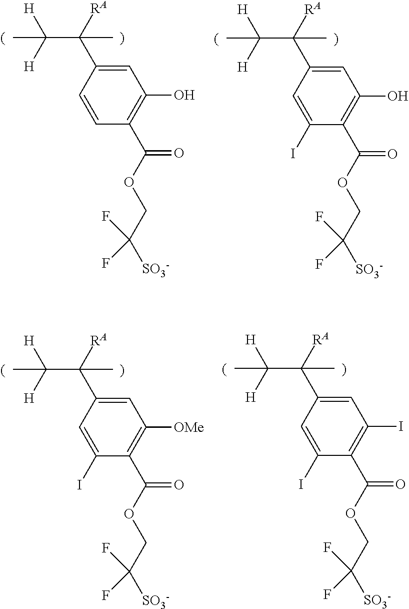

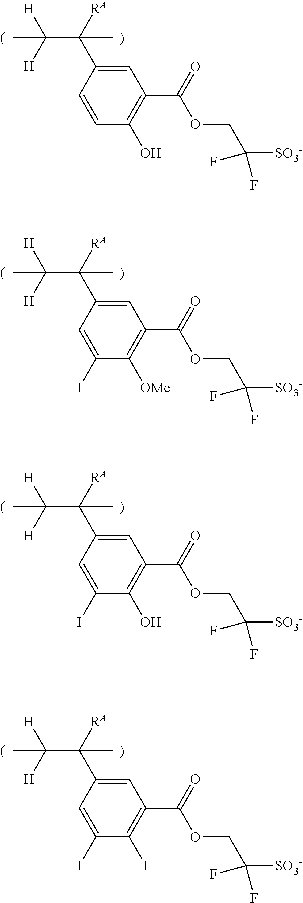

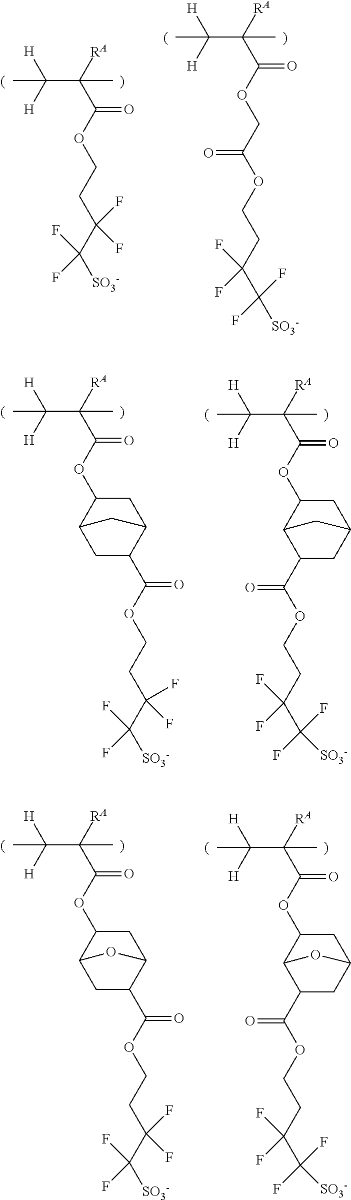

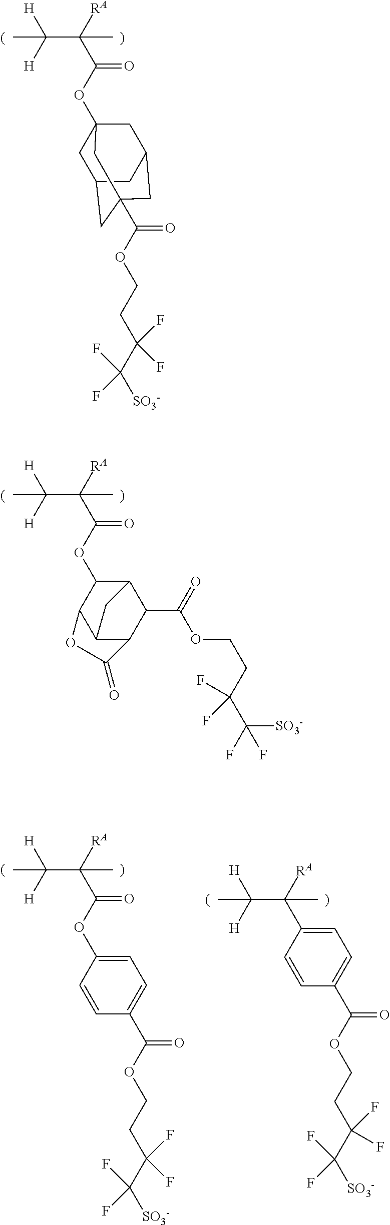

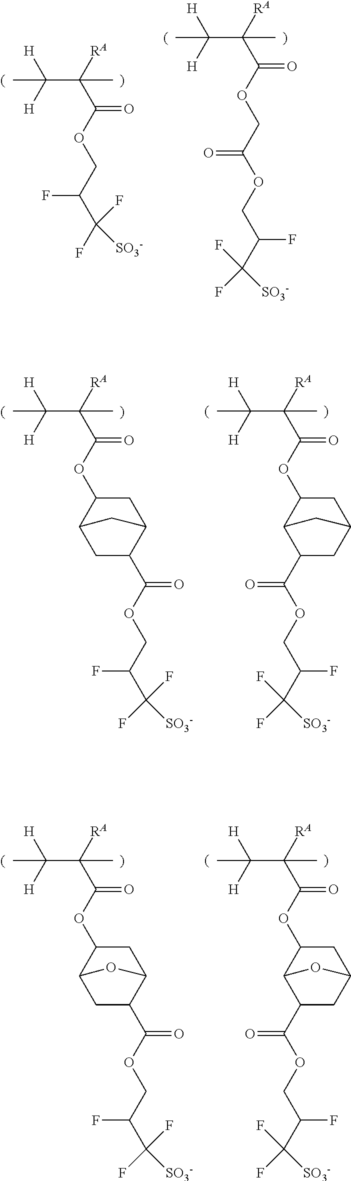

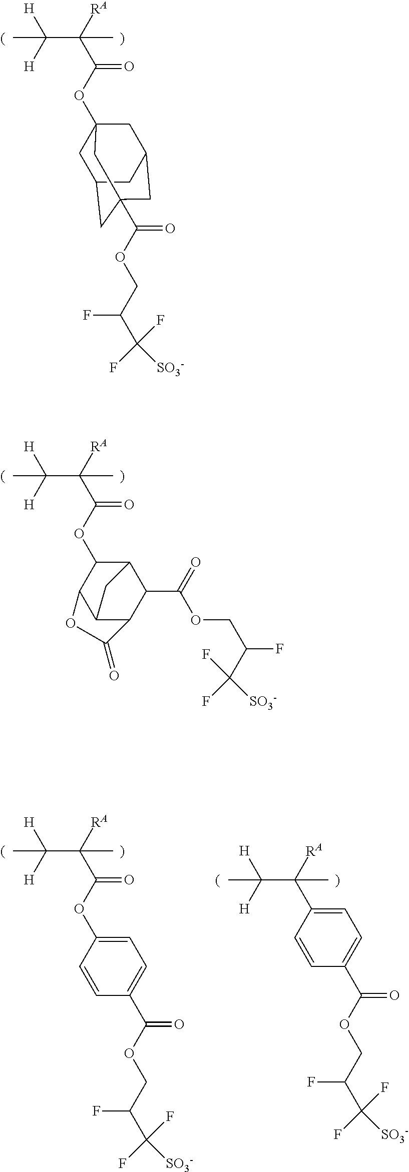

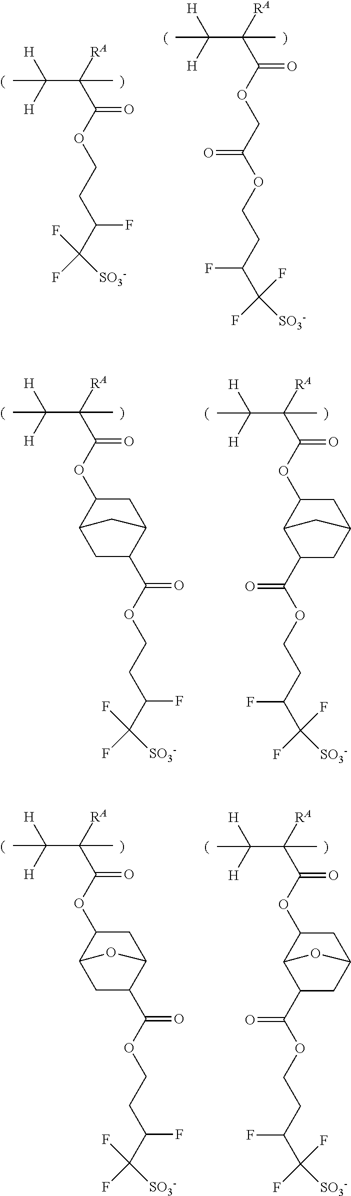

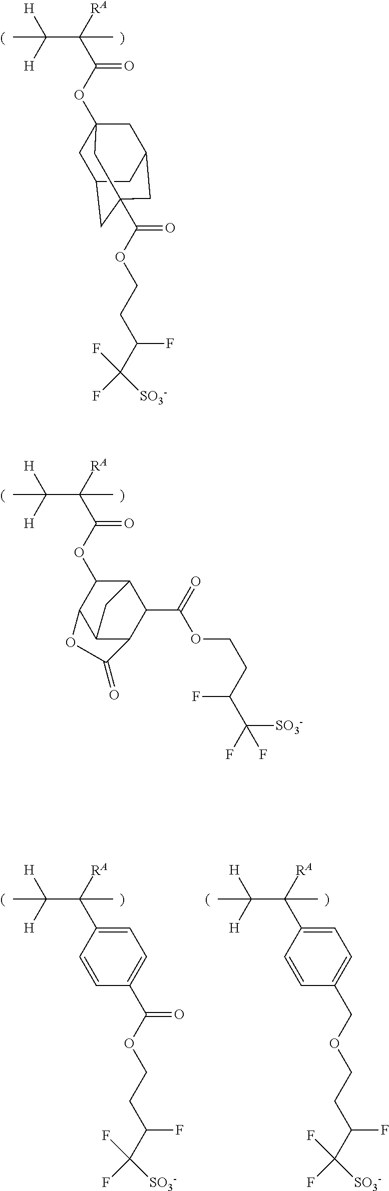

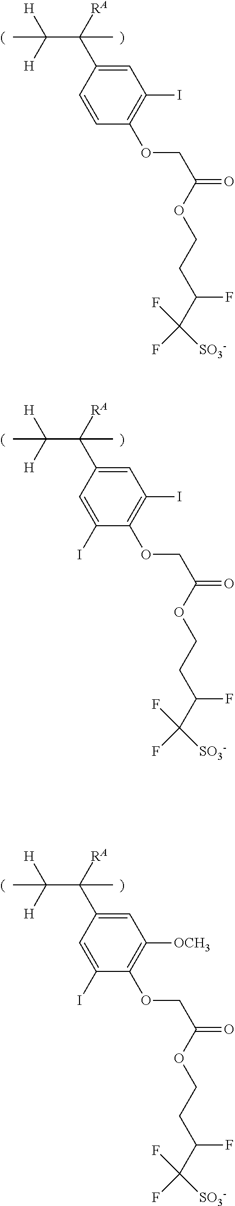

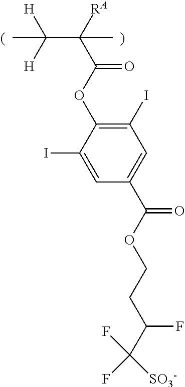

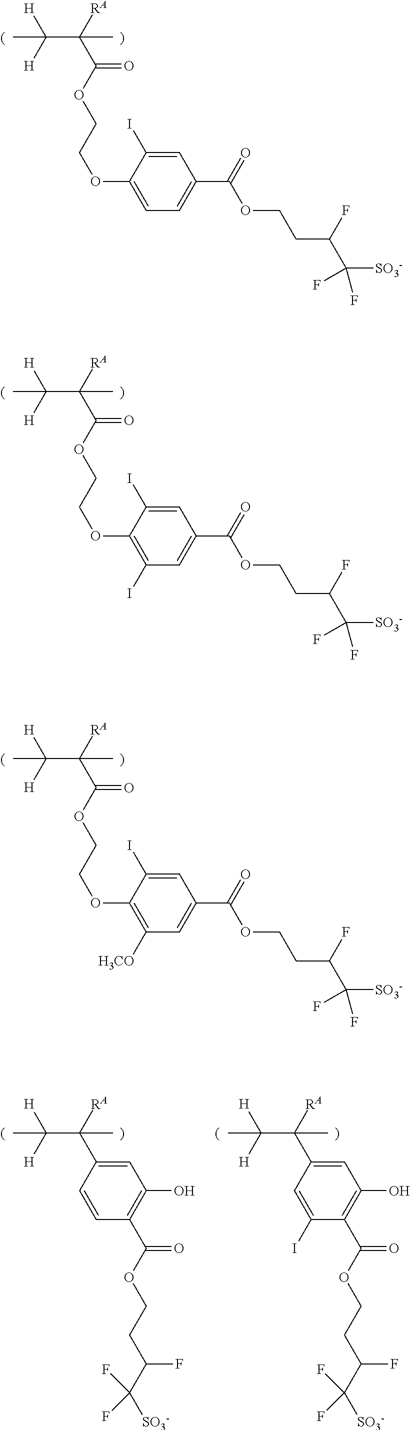

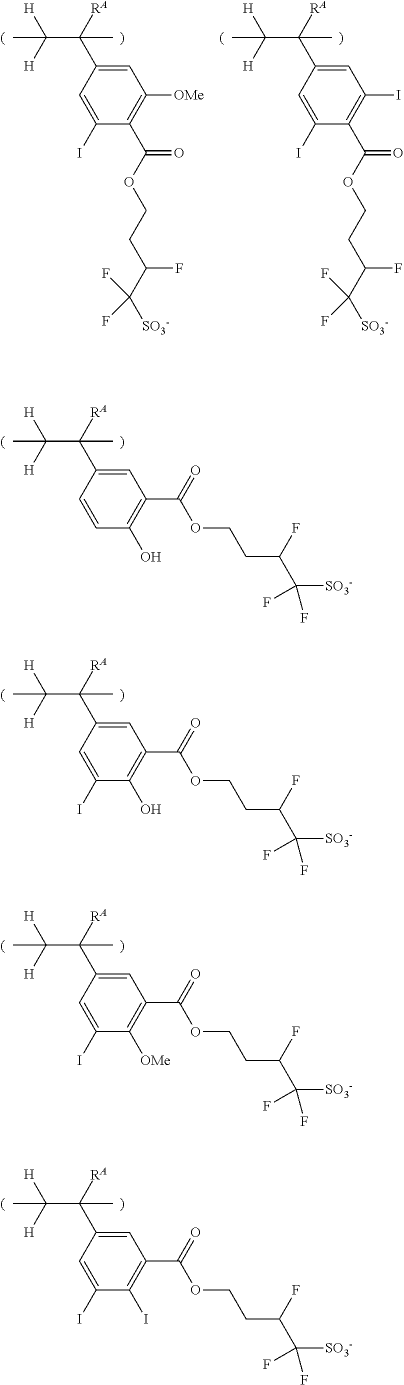

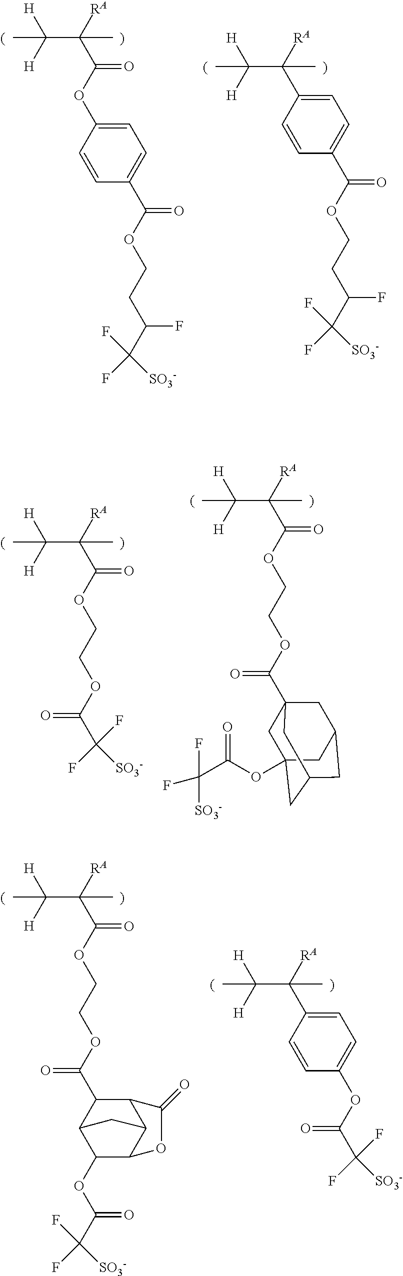

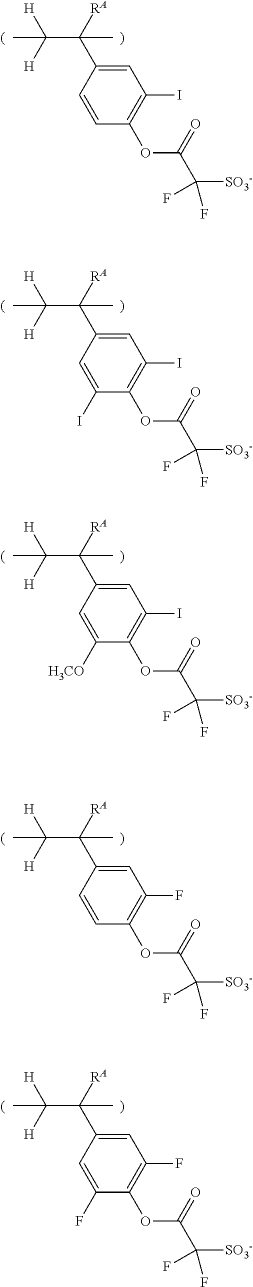

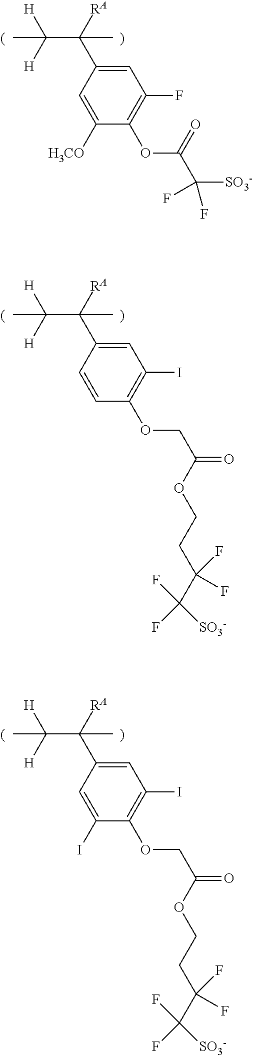

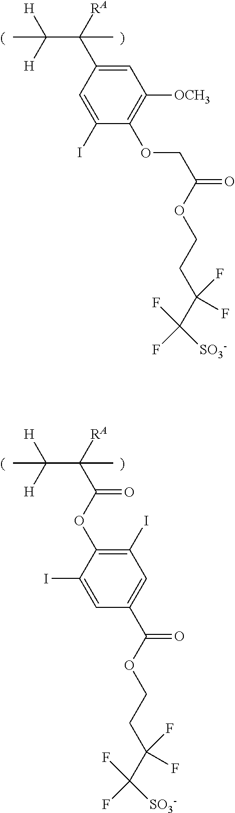

[chemical structure]

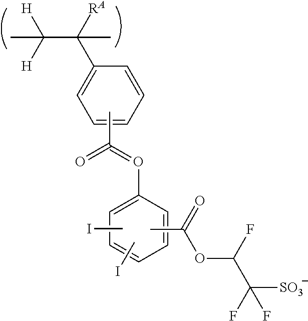

[chemical structure]

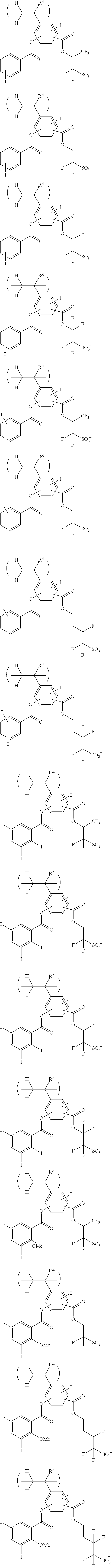

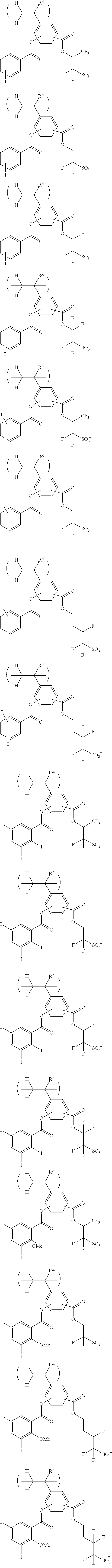

Specific examples of the anion of the repeating unit-c3 include, but are not limited to, those shown below. Note that, in the following formulae, R^{A} is as defined above.

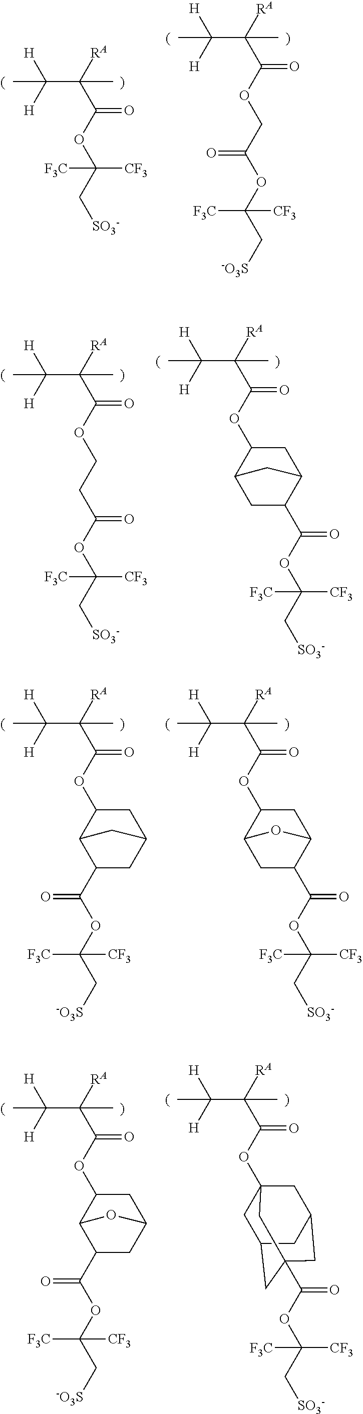

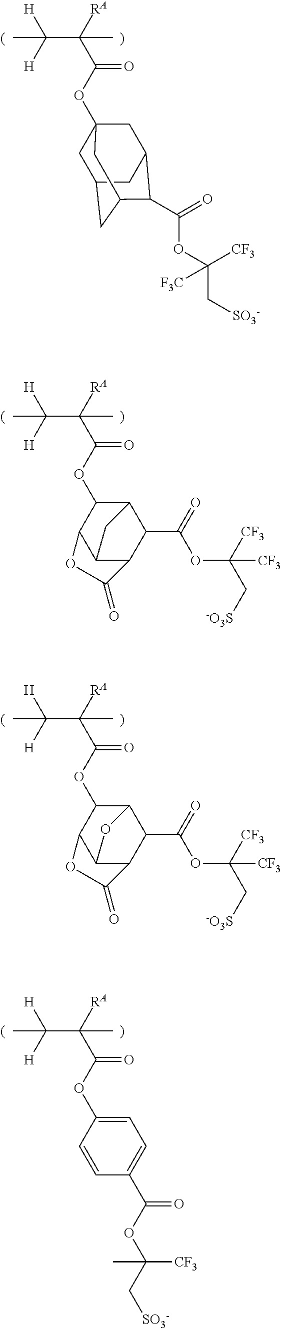

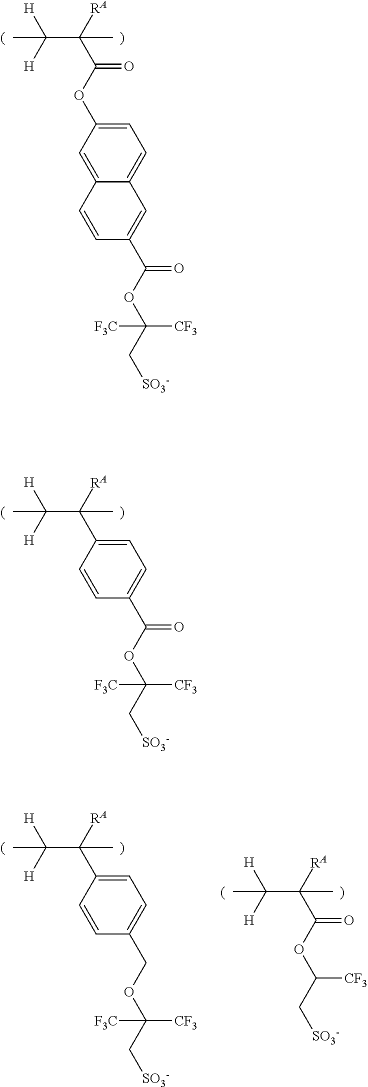

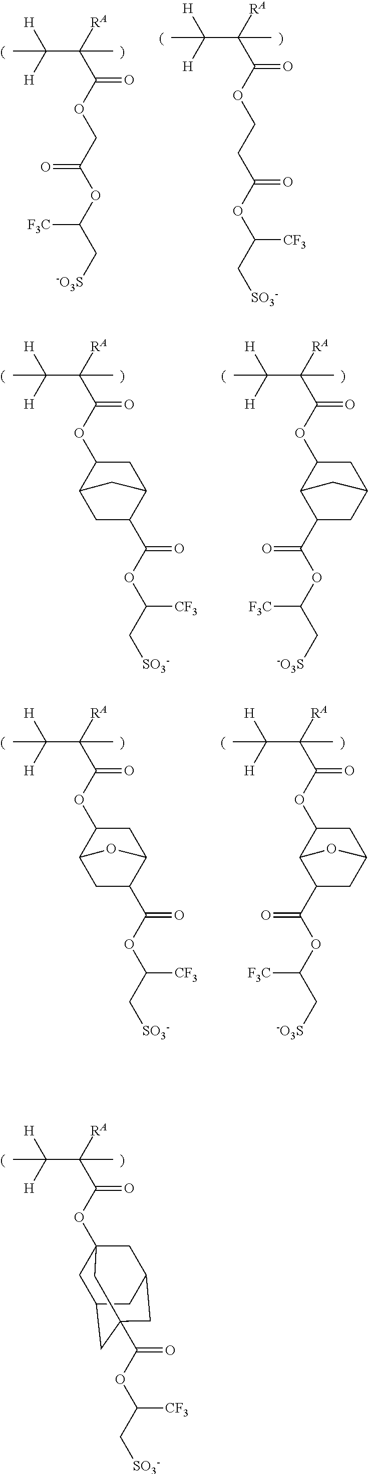

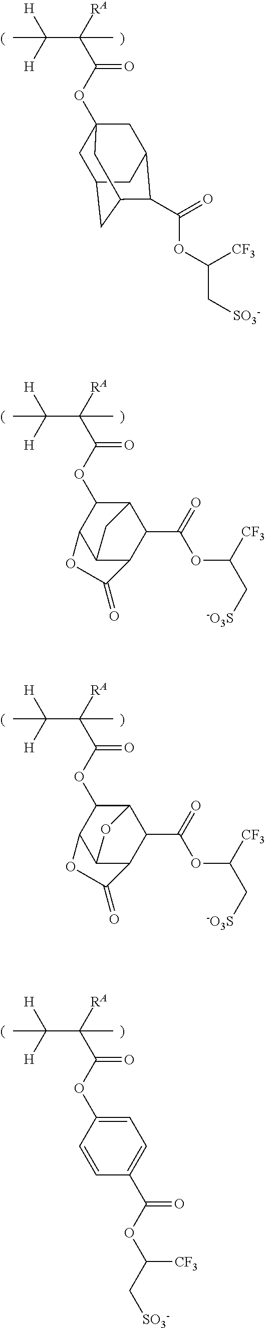

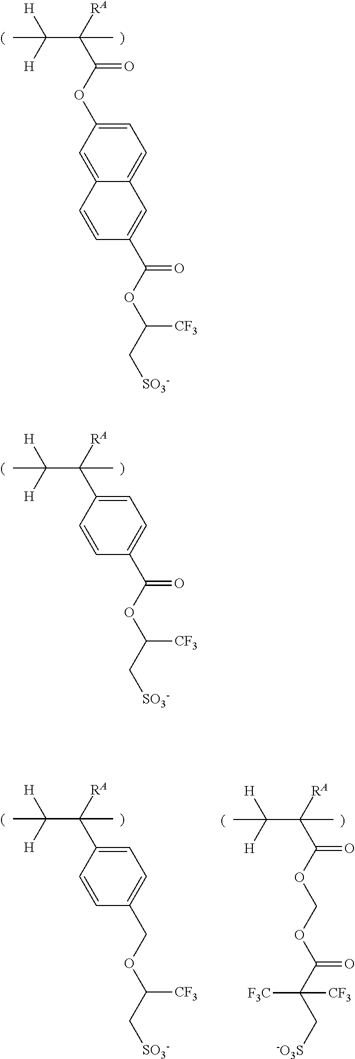

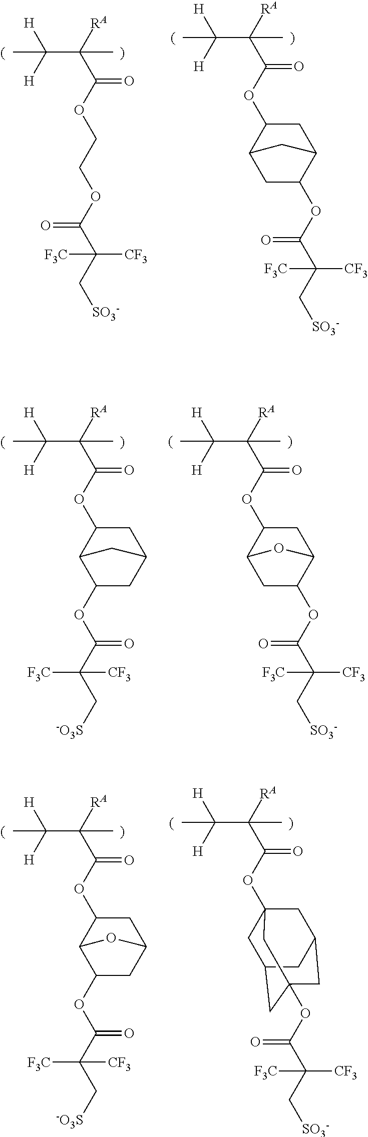

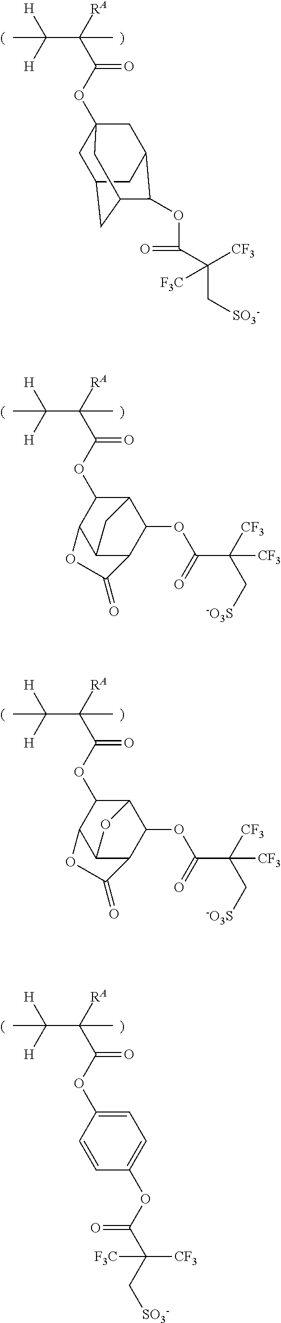

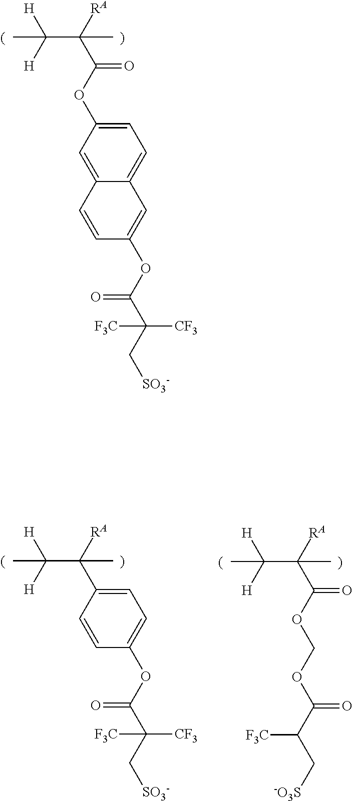

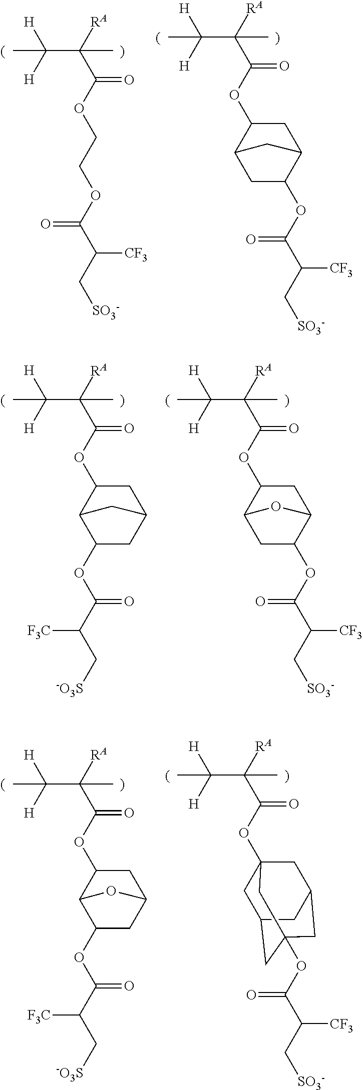

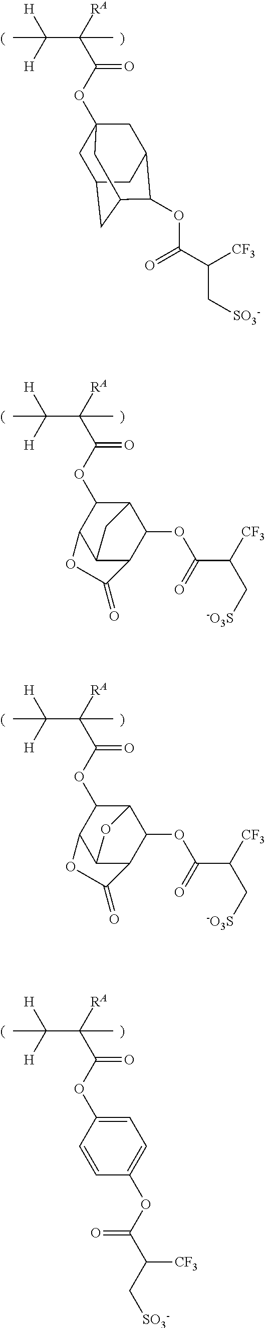

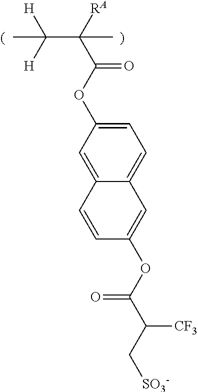

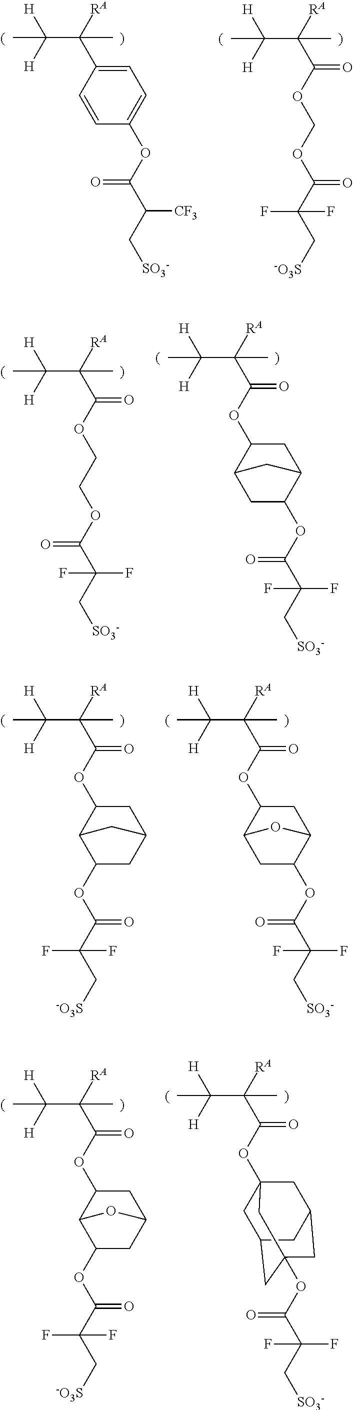

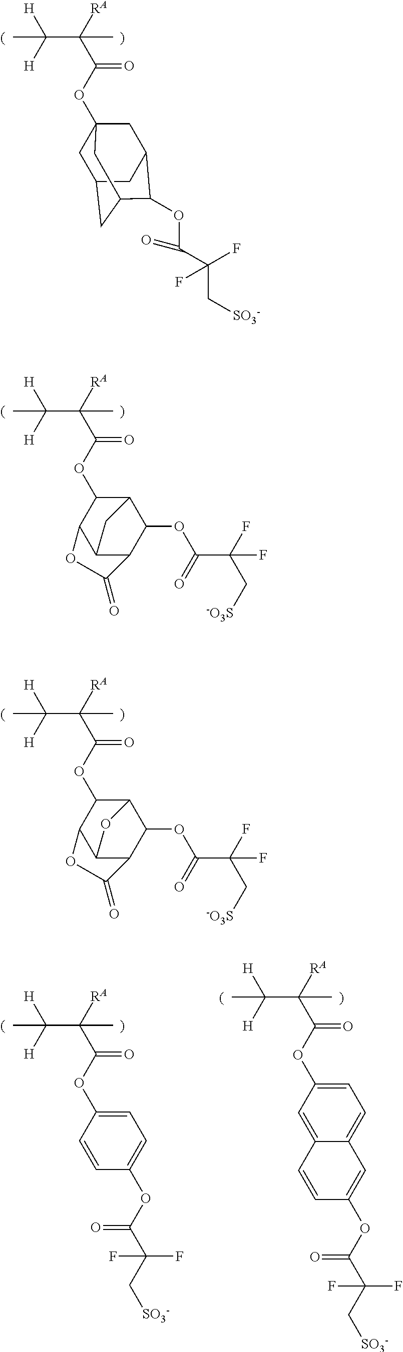

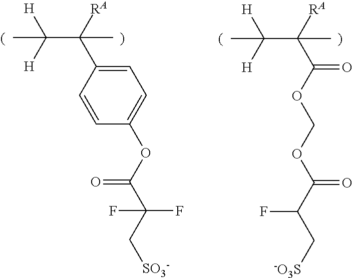

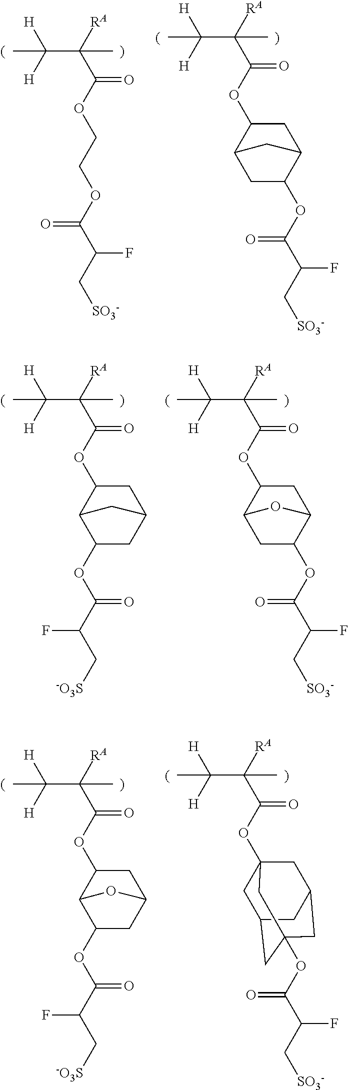

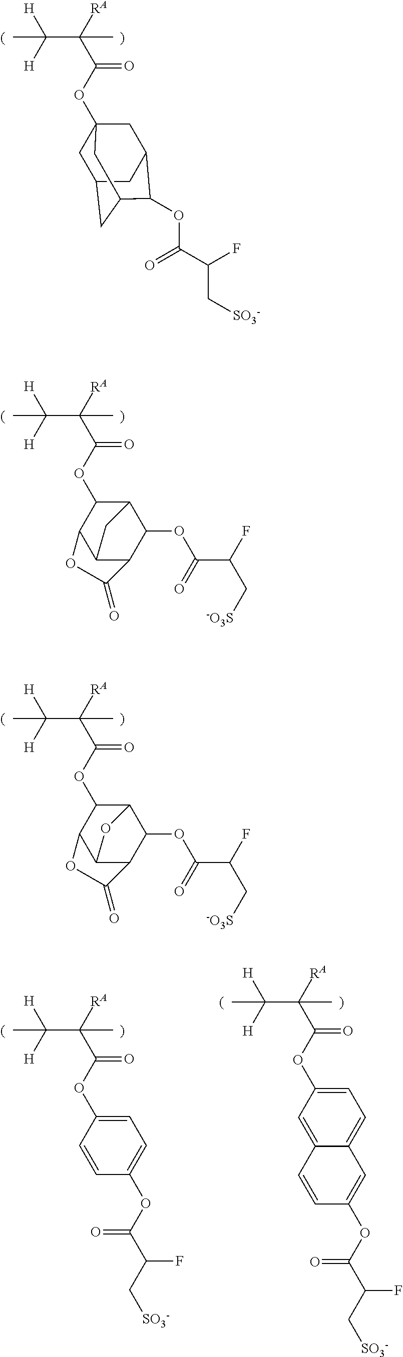

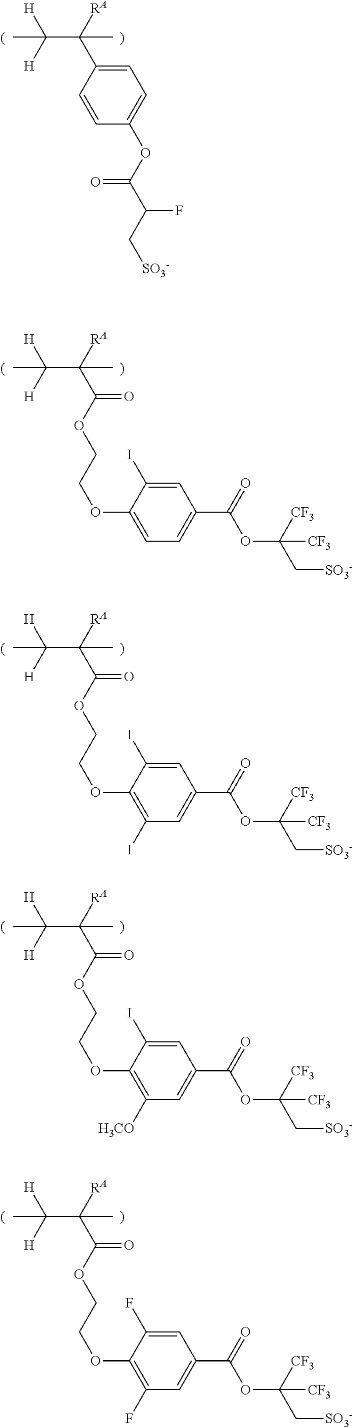

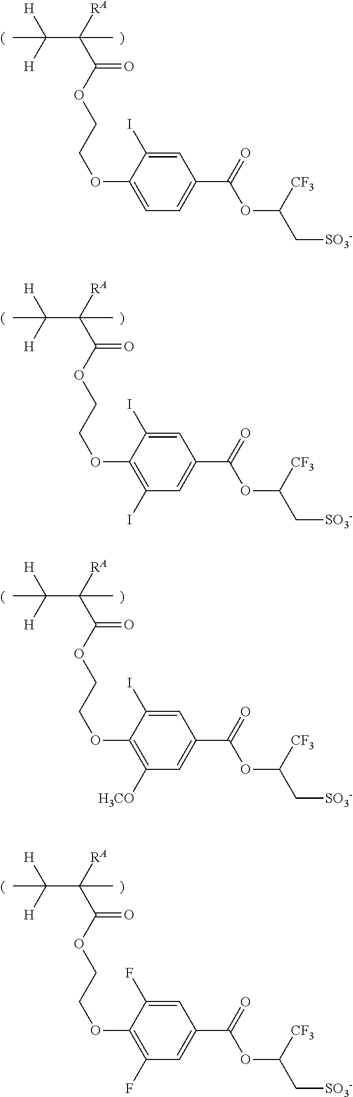

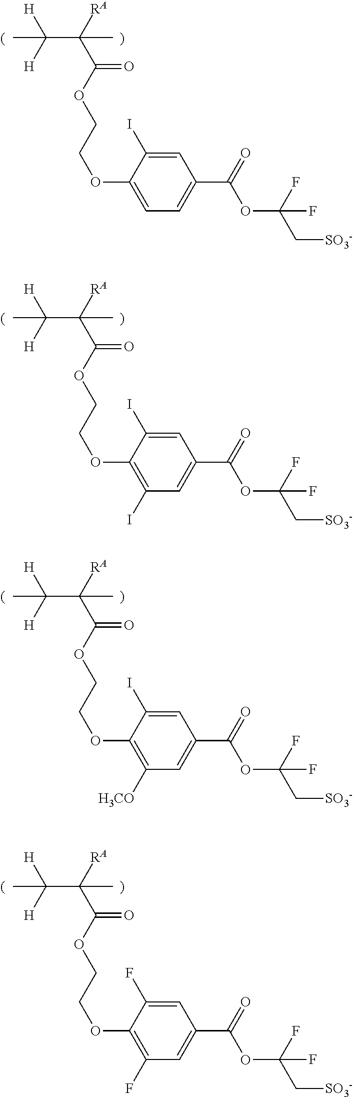

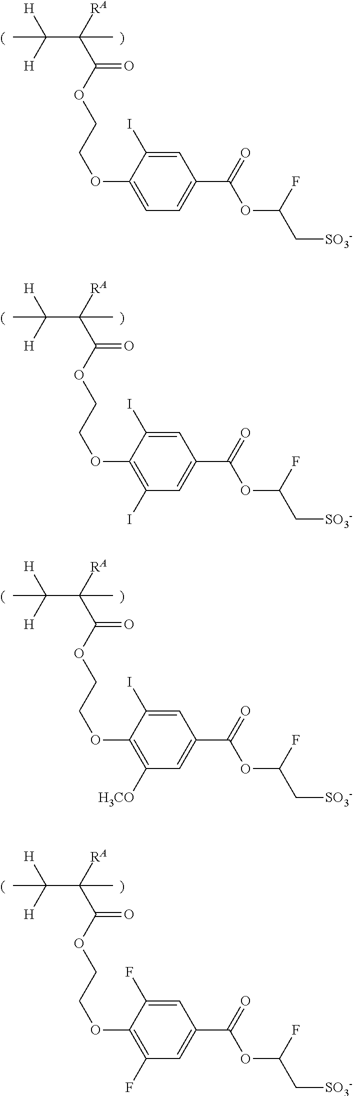

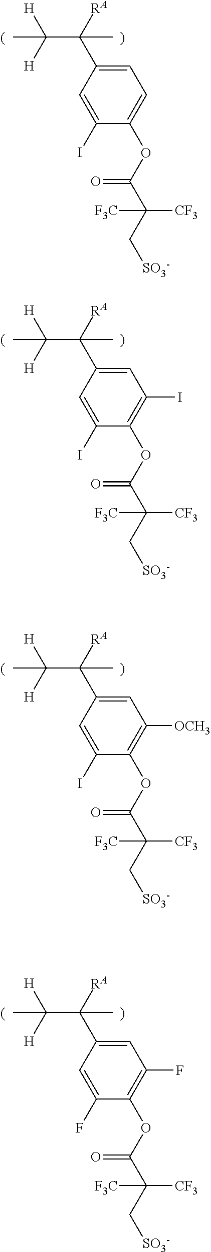

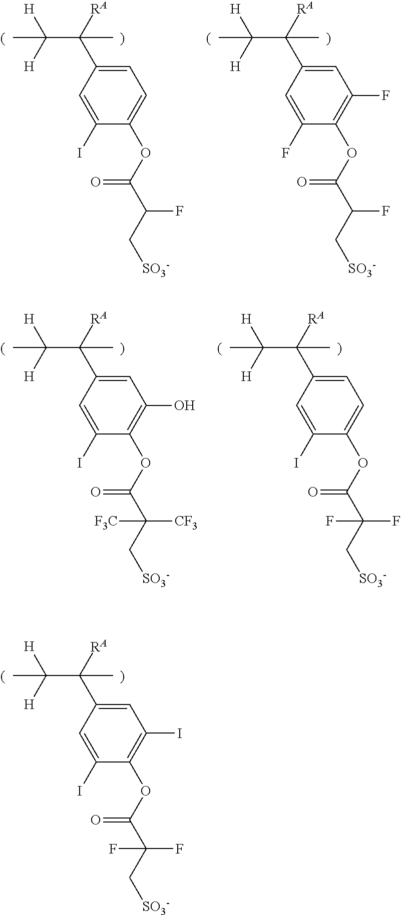

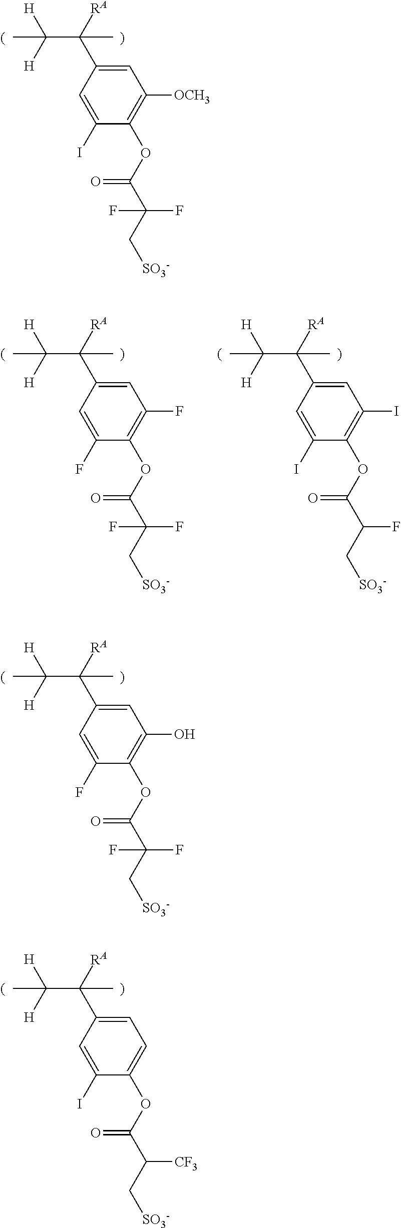

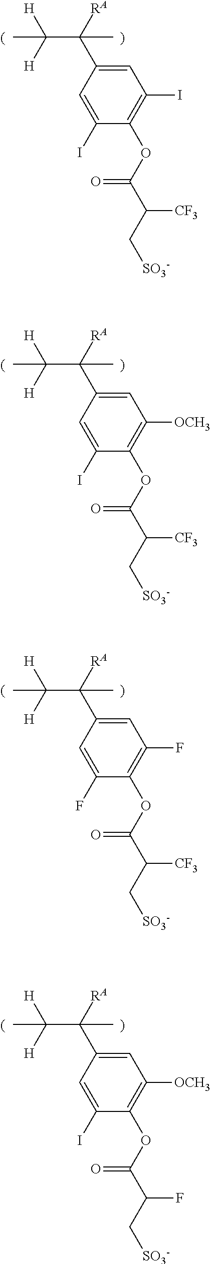

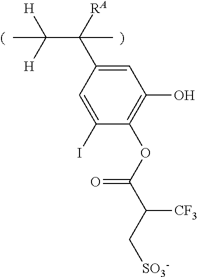

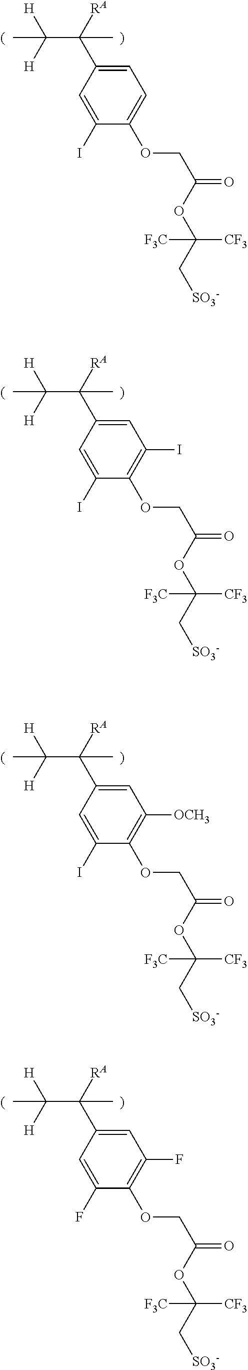

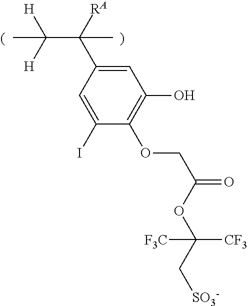

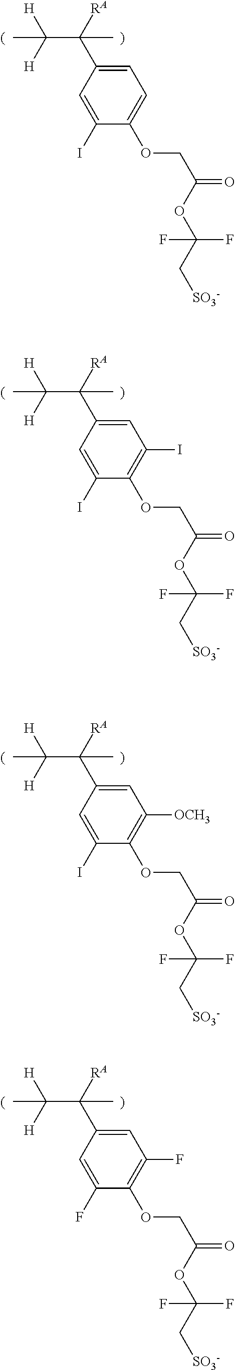

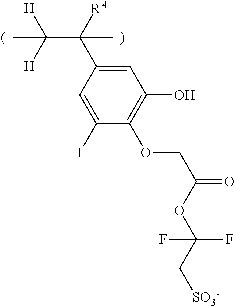

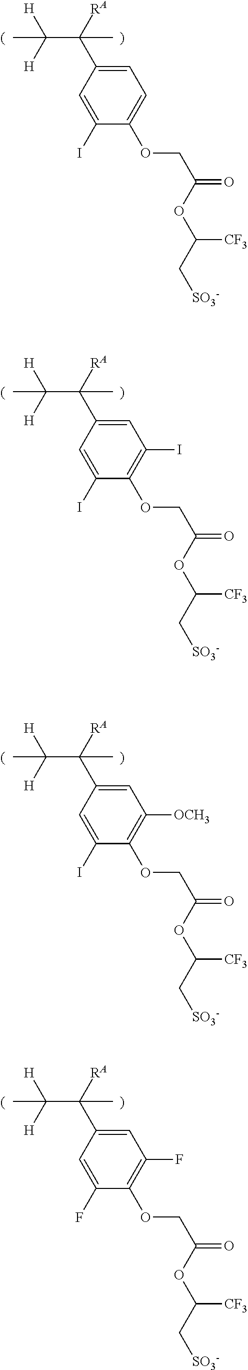

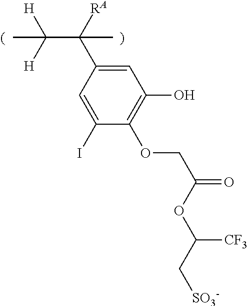

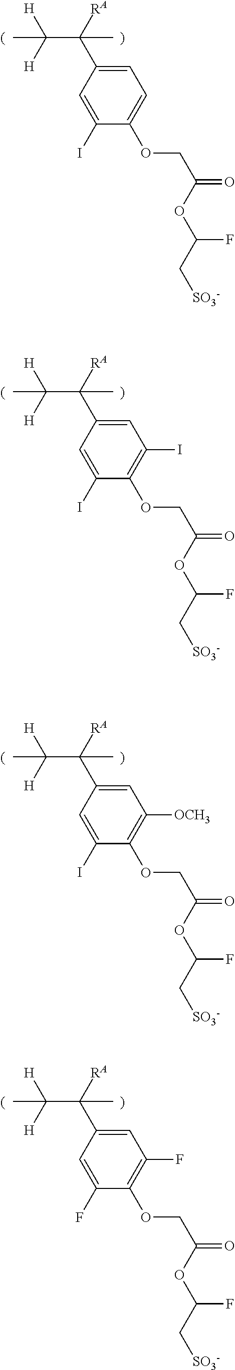

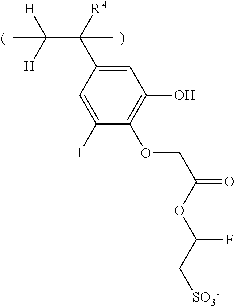

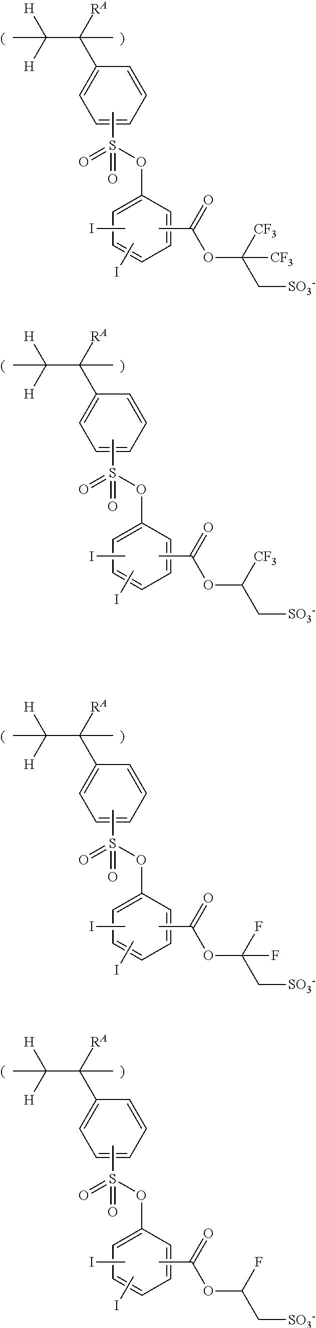

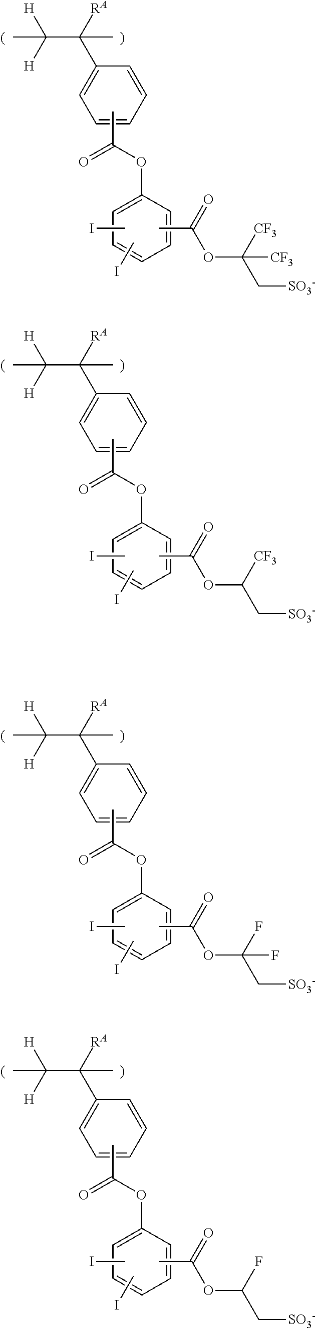

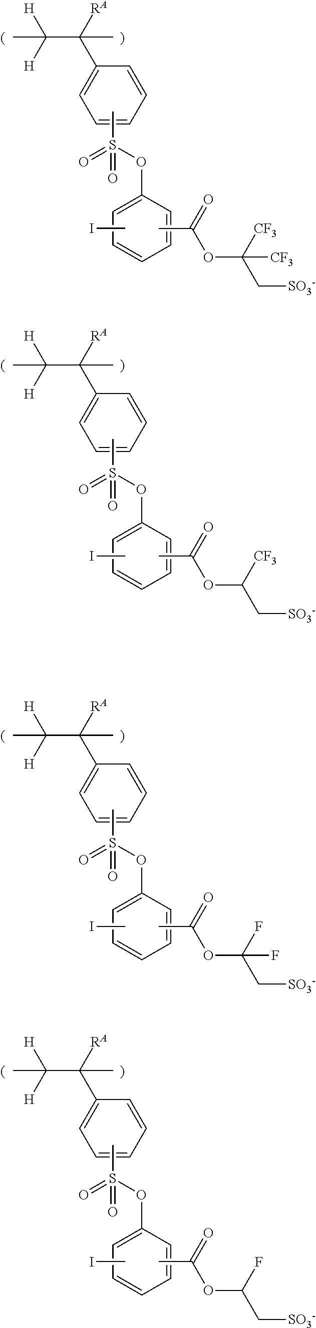

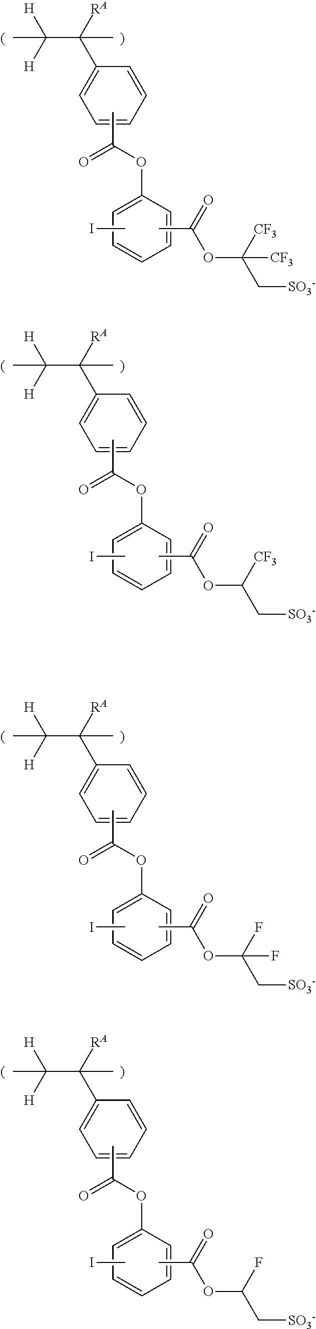

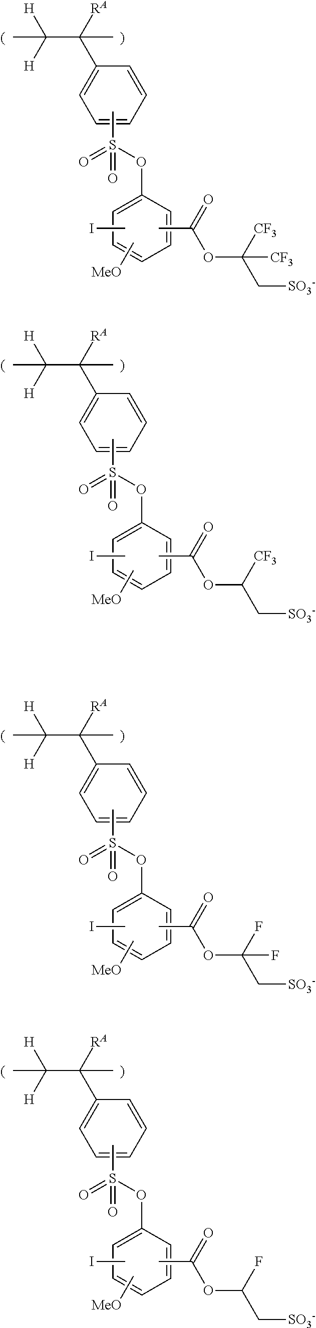

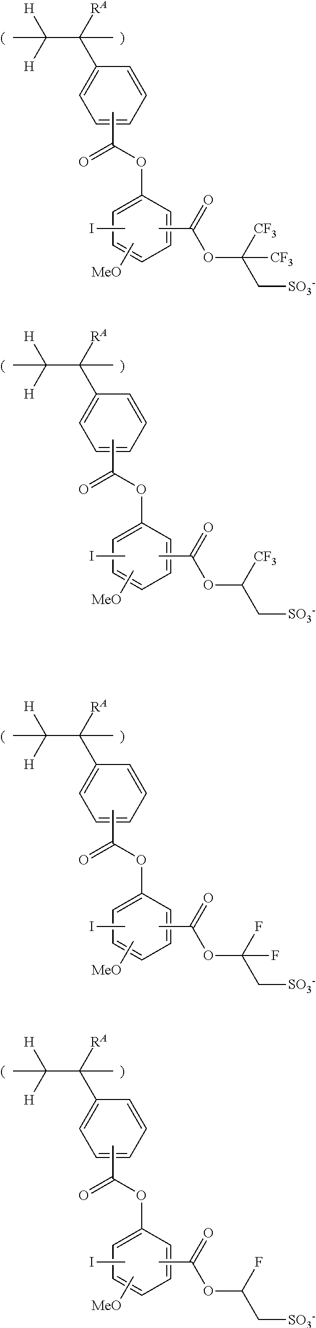

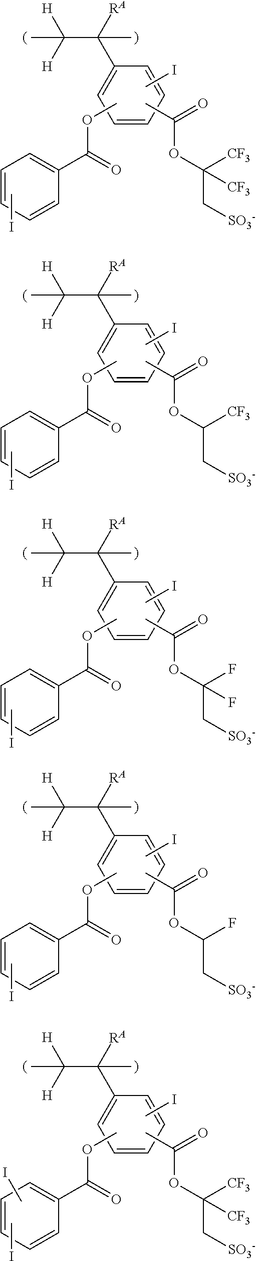

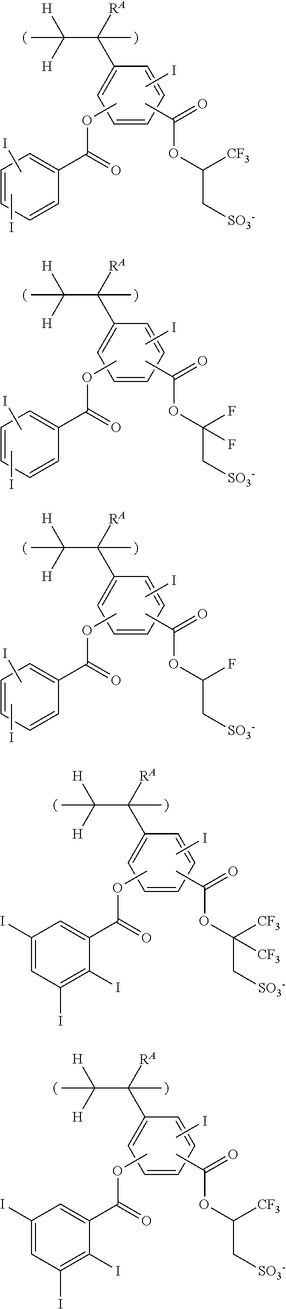

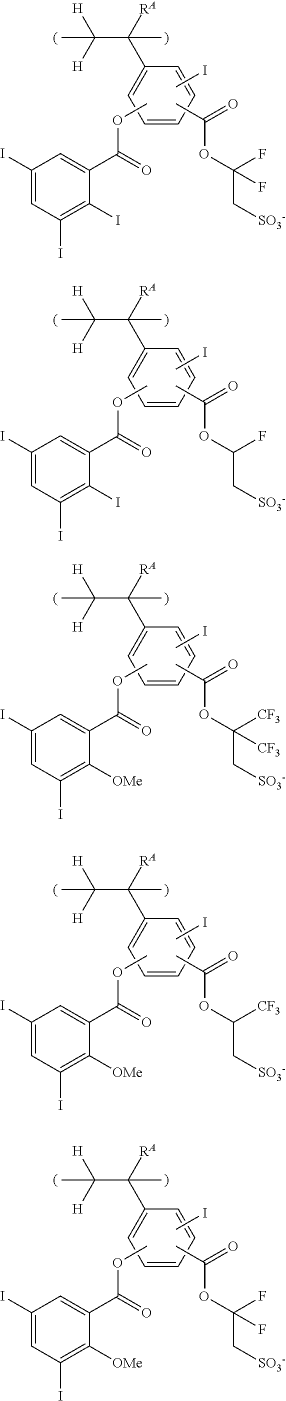

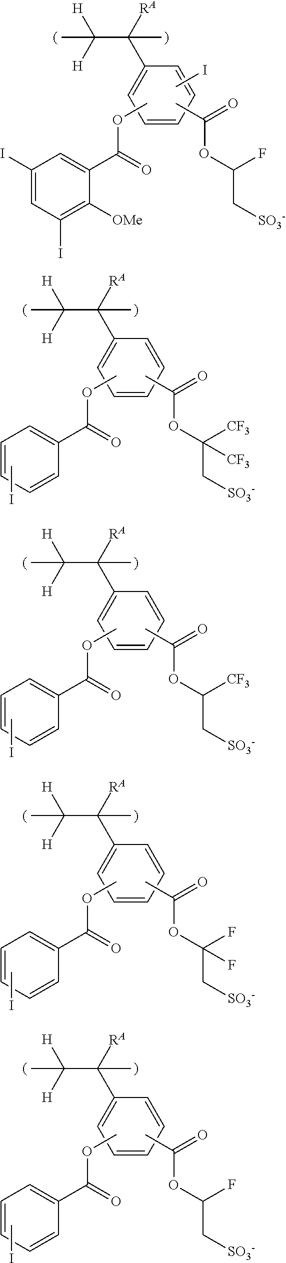

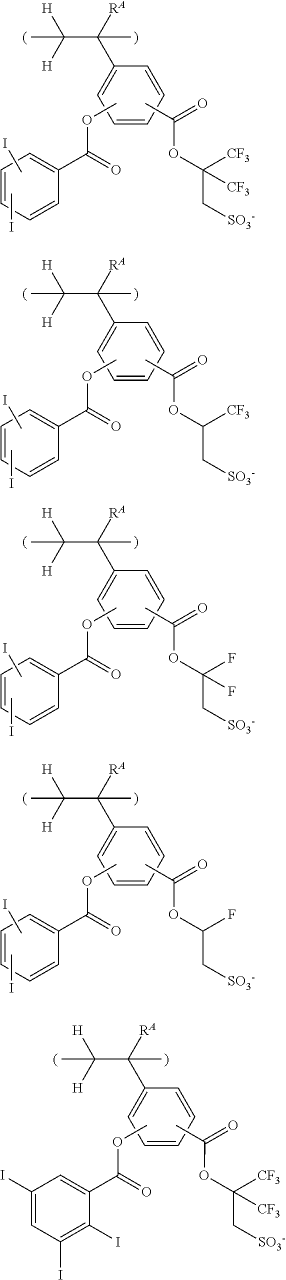

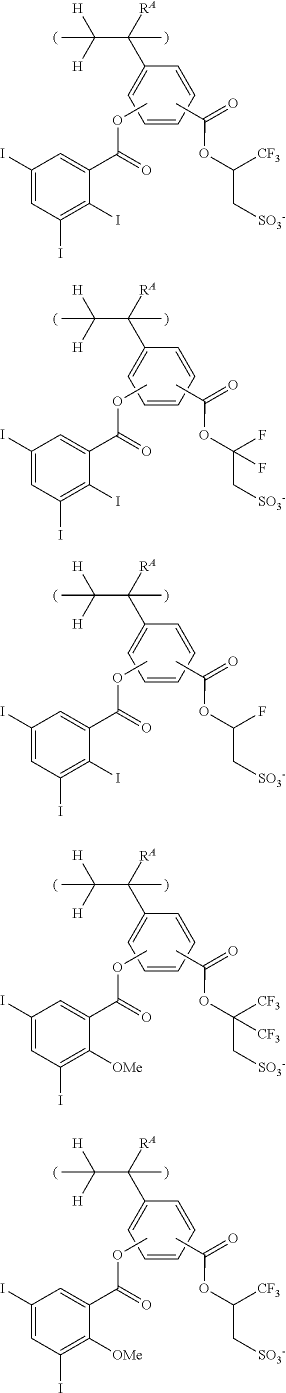

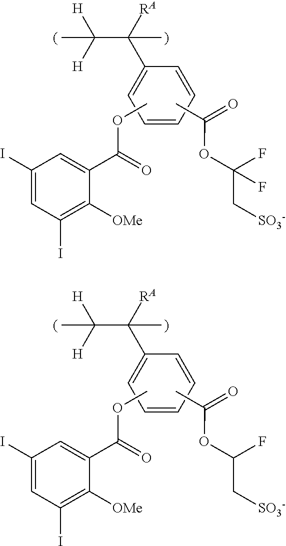

Specific examples of the anion of the repeating unit-c4 include, but are not limited to, those shown below. Note that, in the following formulae, R^{A} is as defined above.

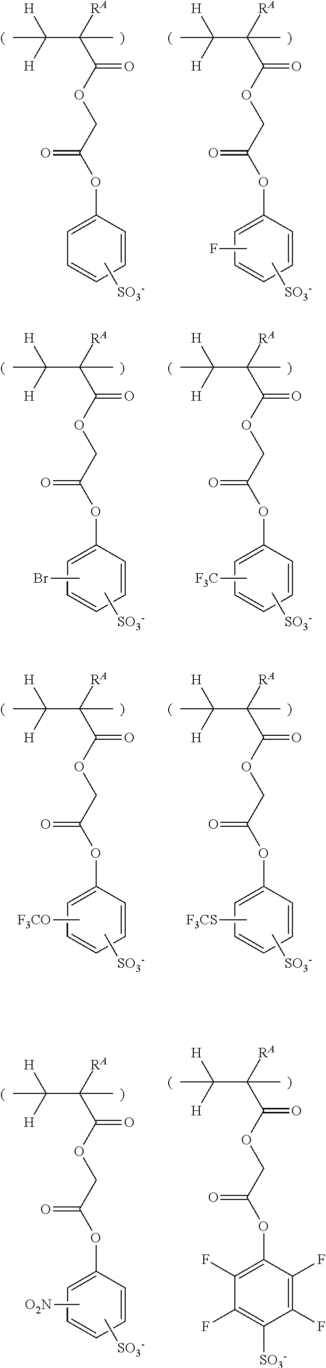

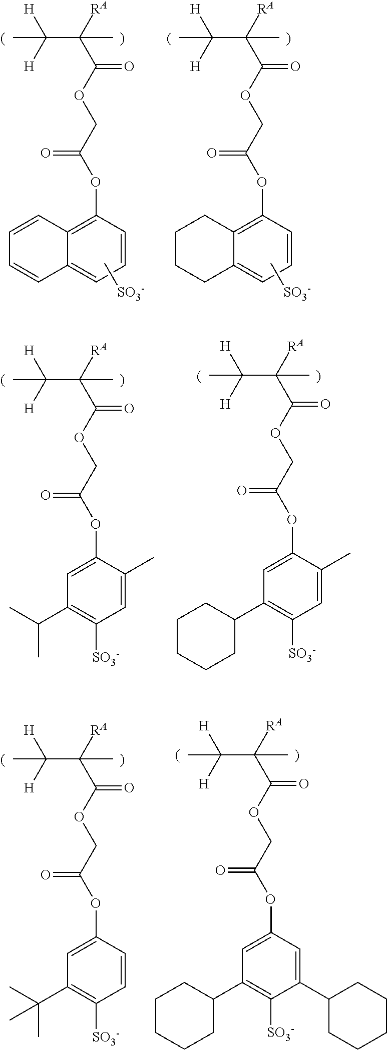

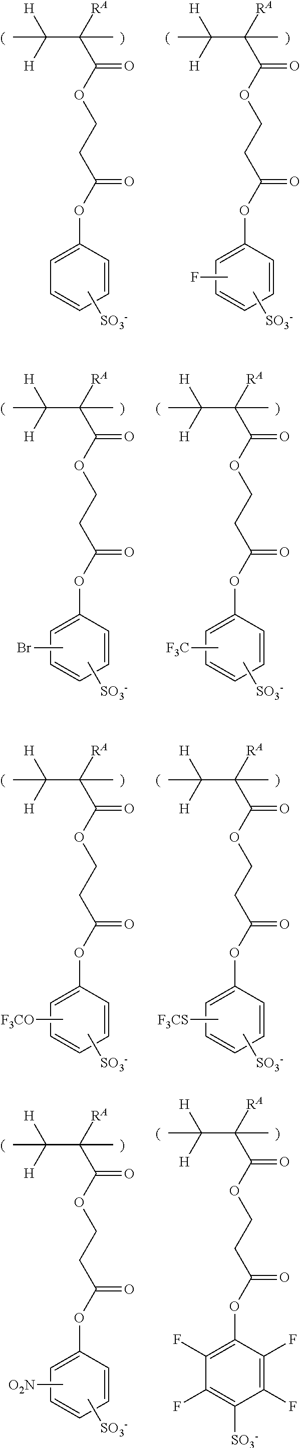

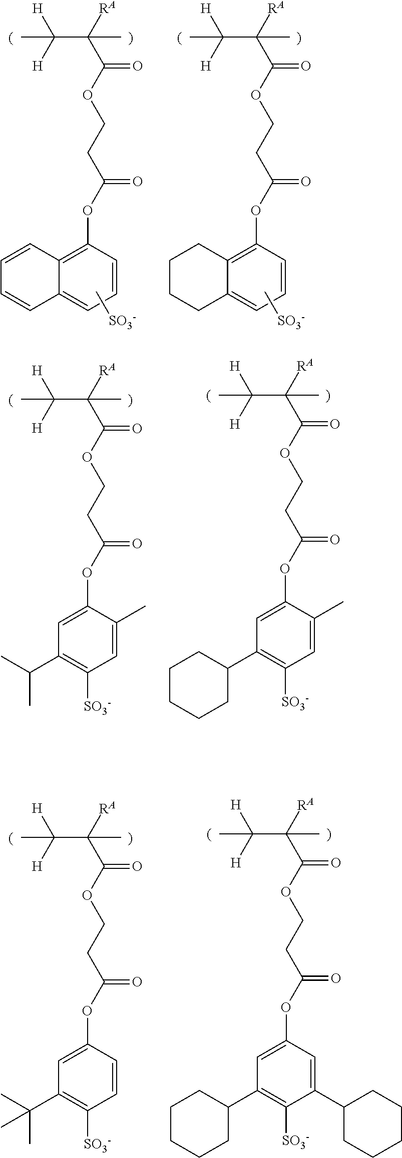

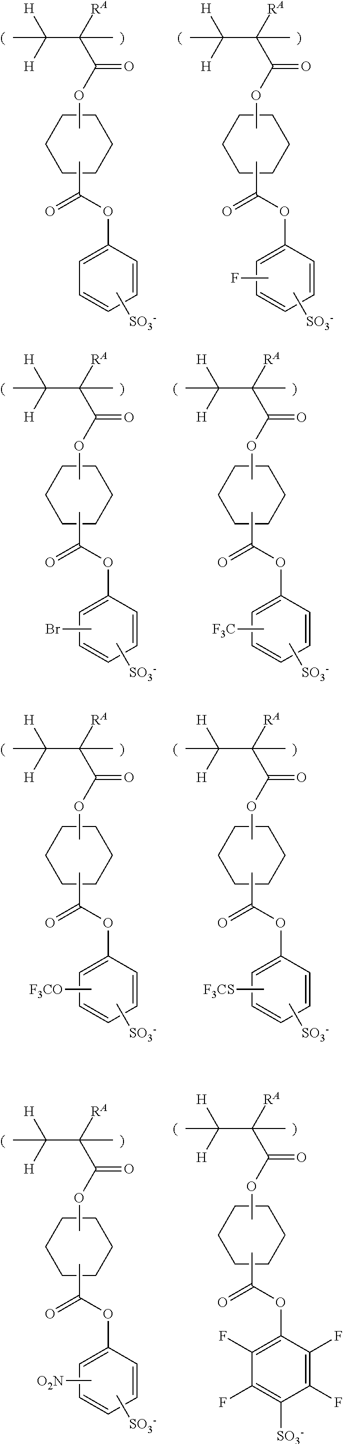

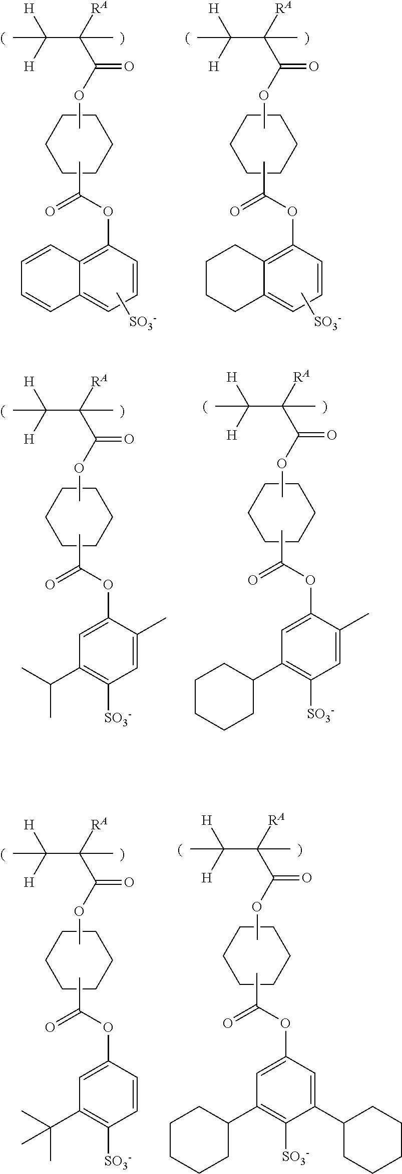

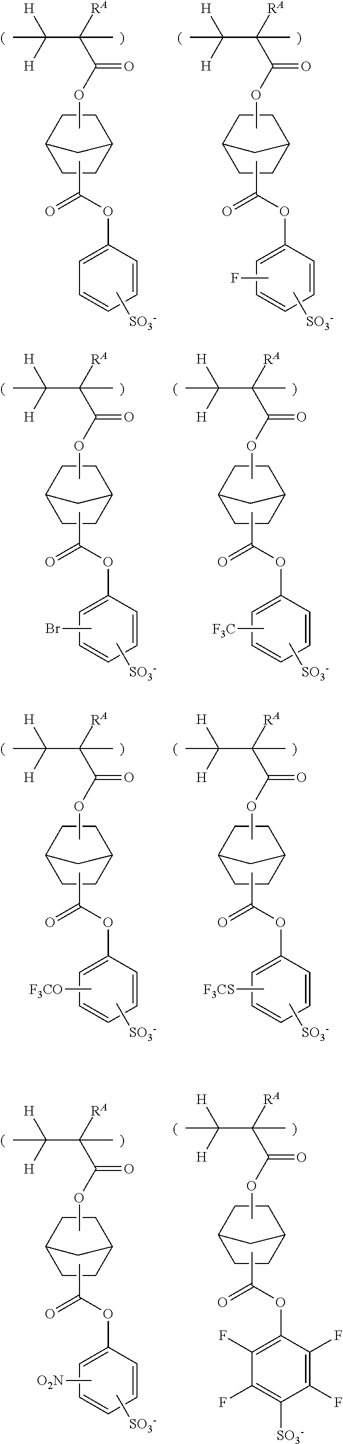

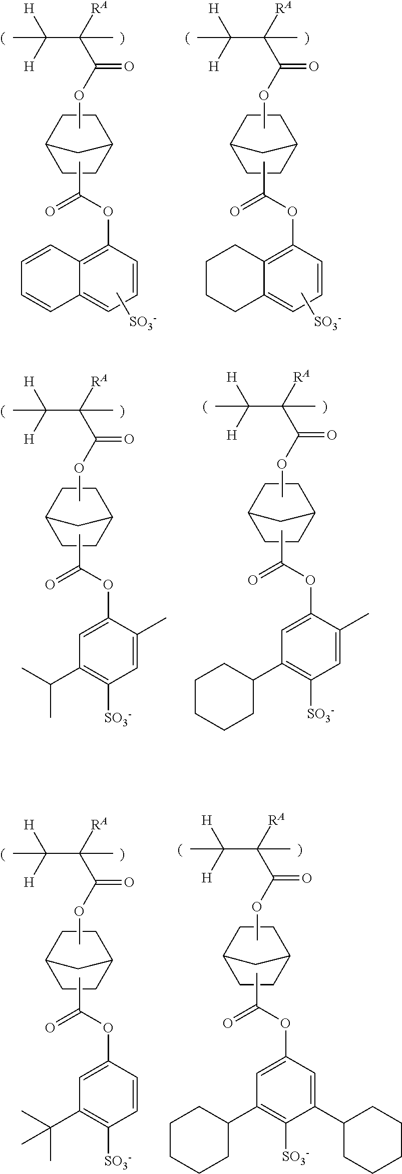

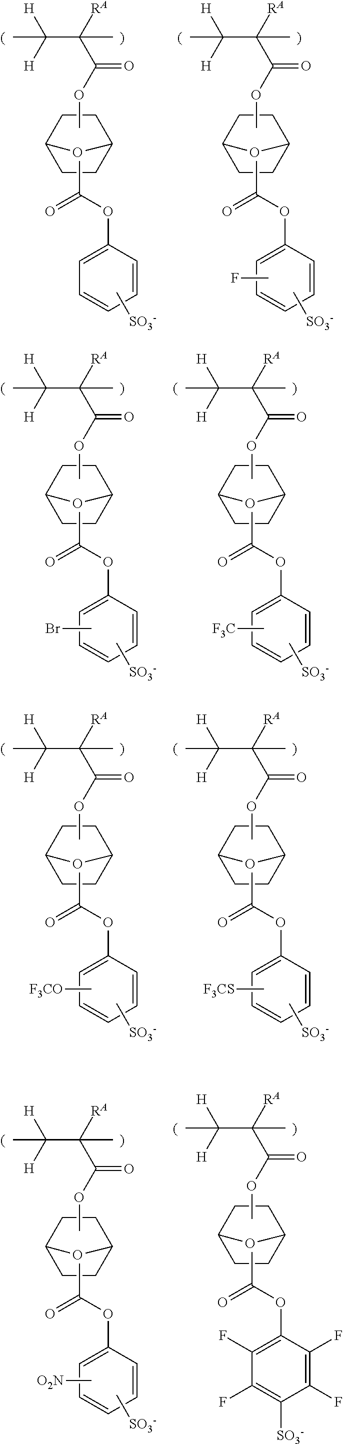

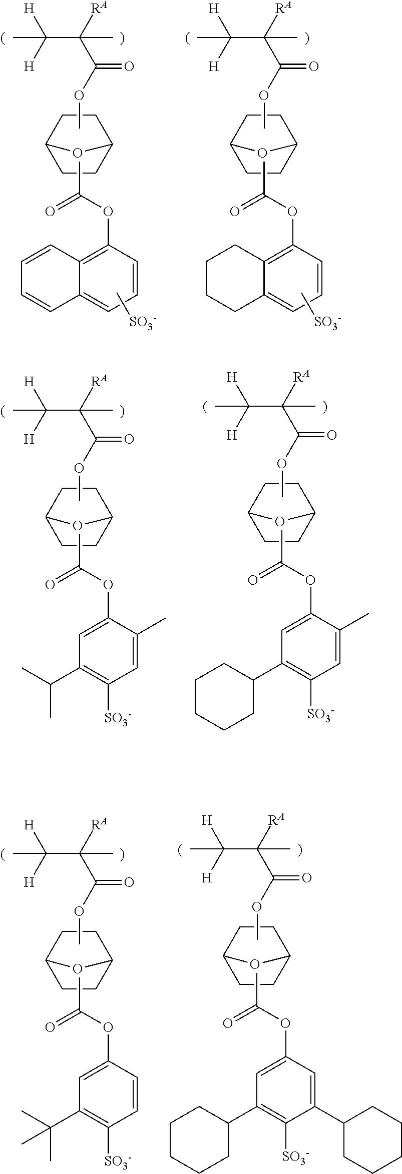

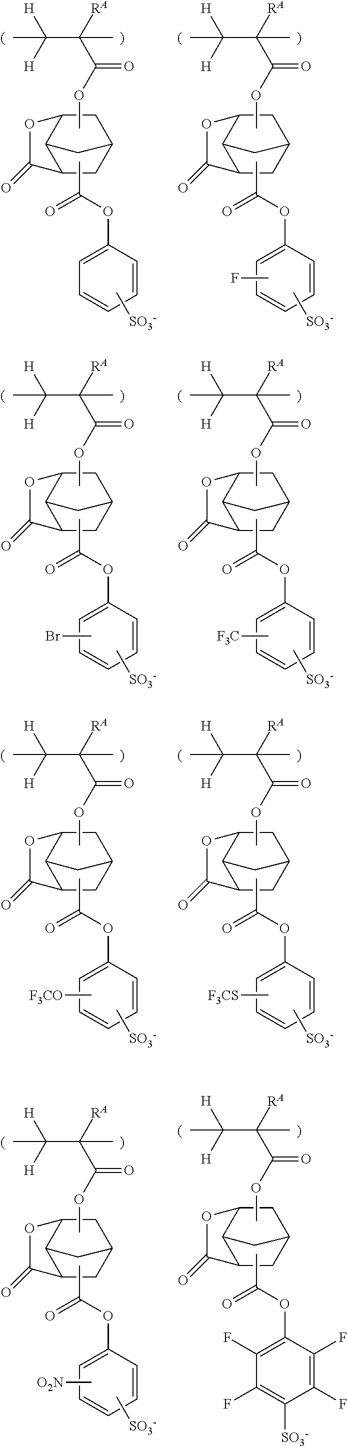

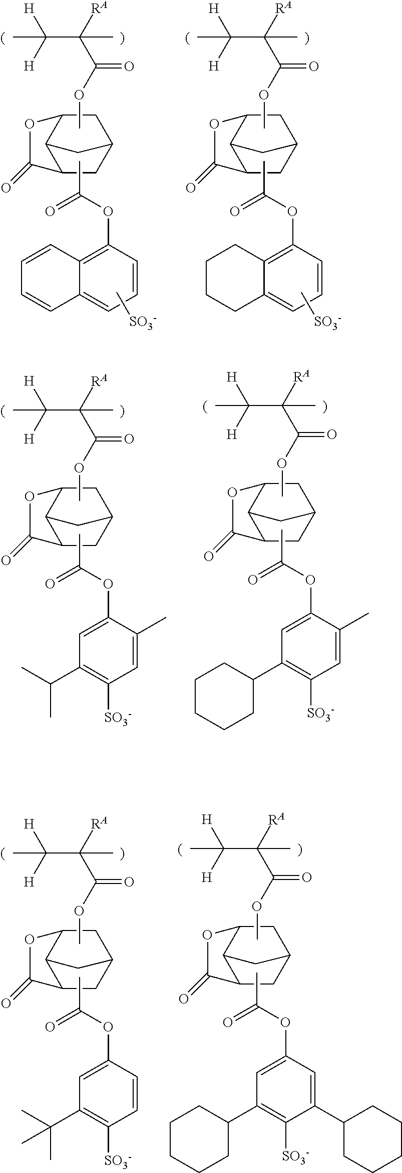

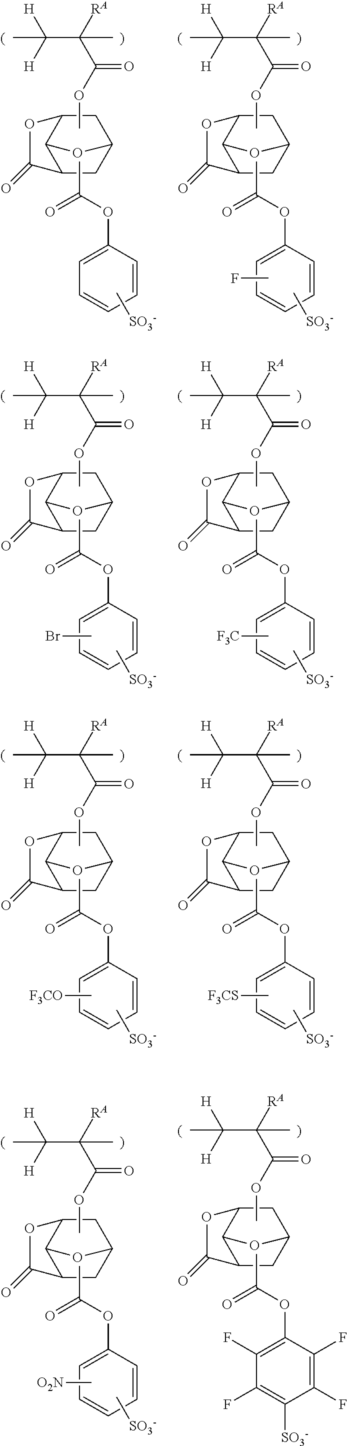

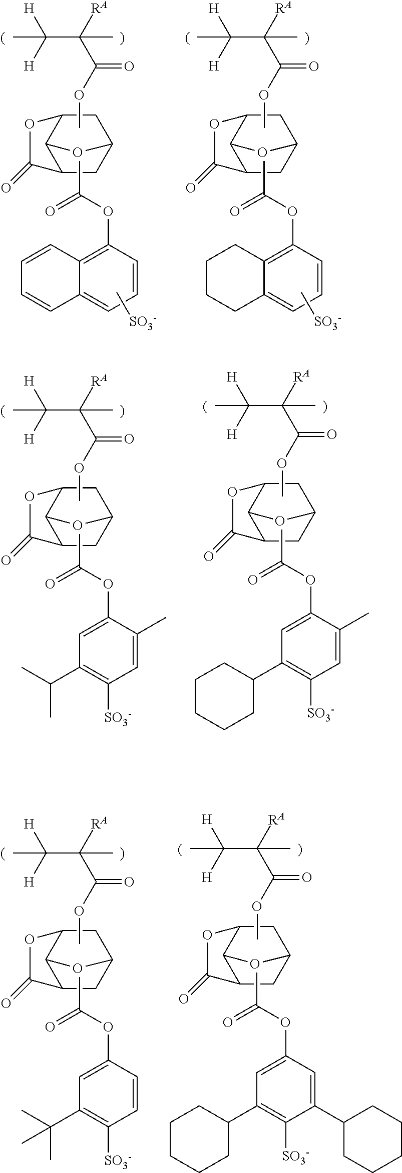

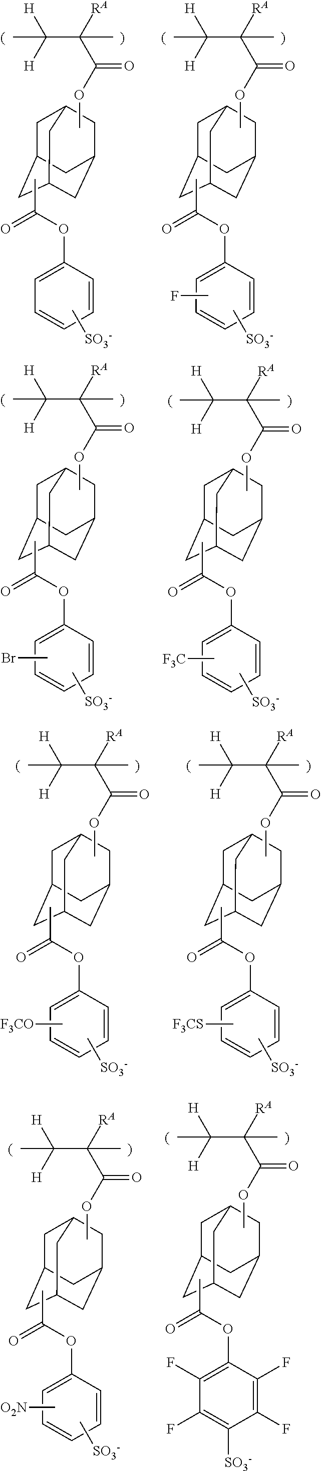

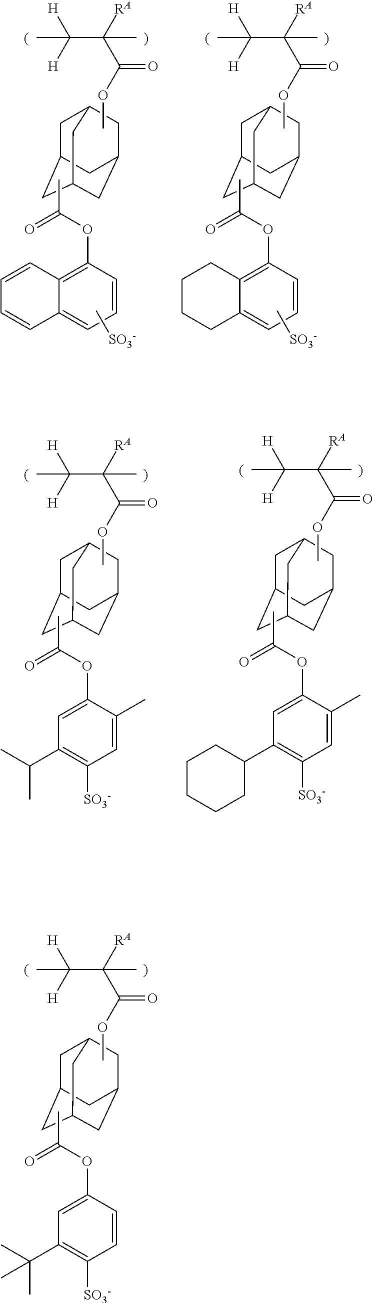

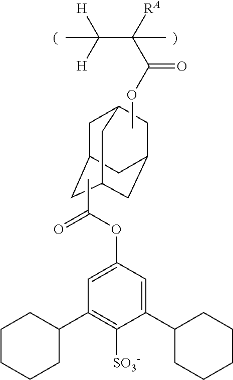

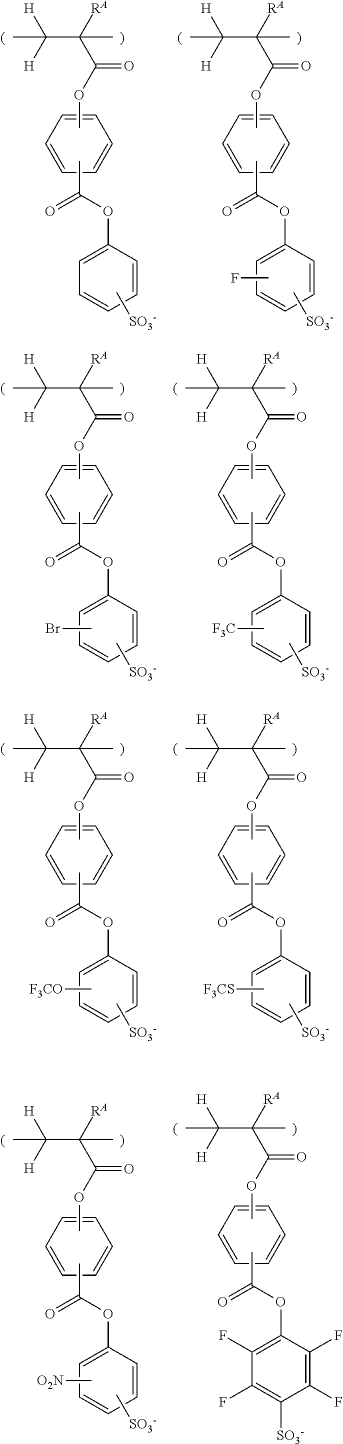

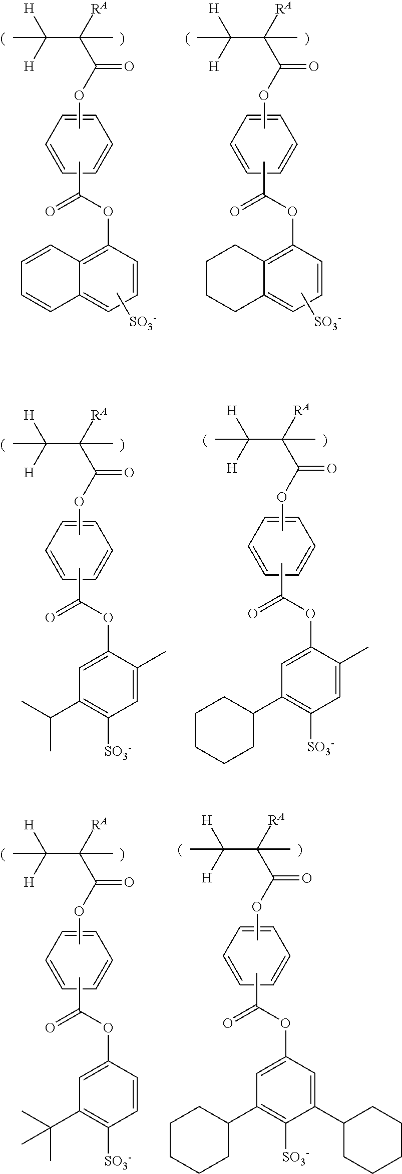

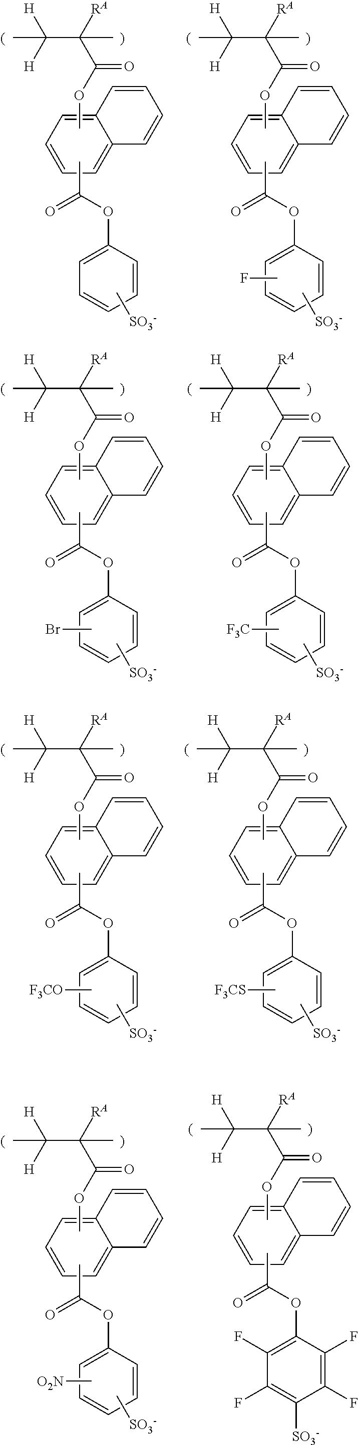

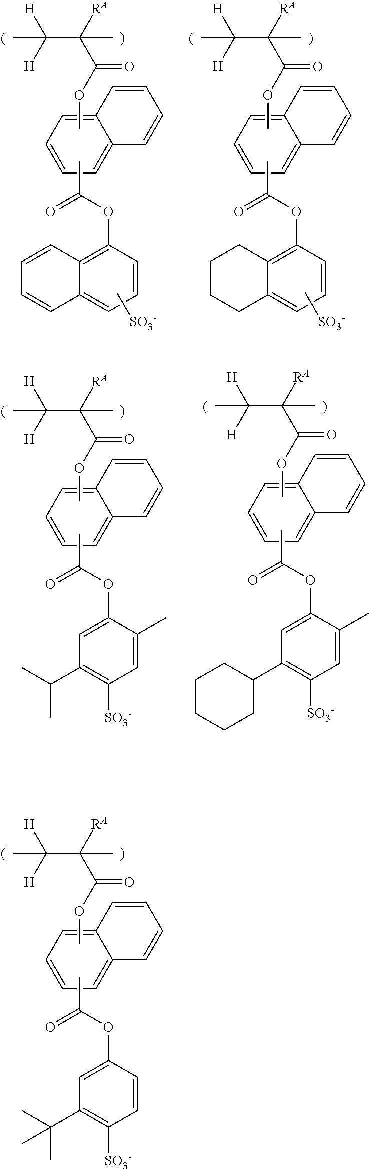

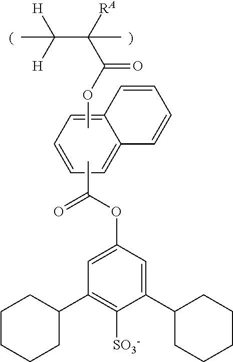

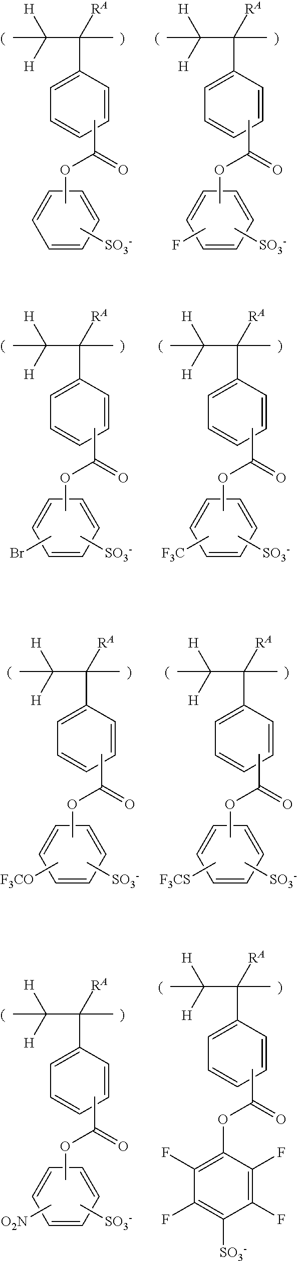

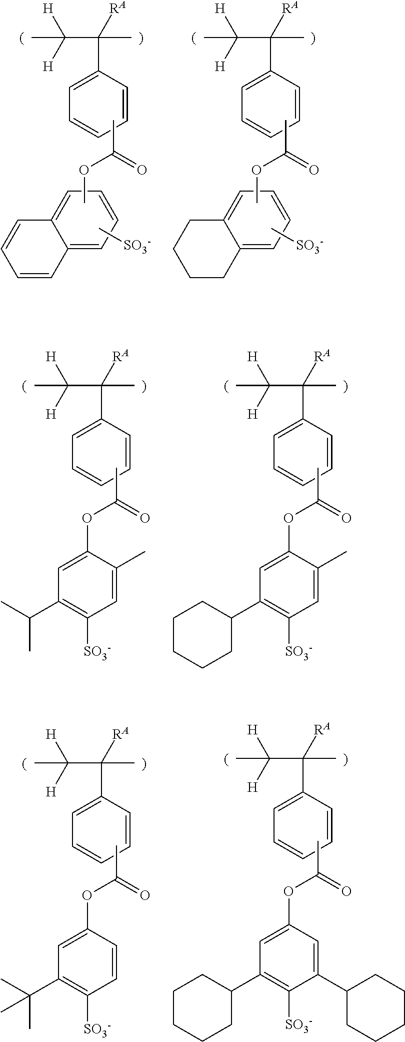

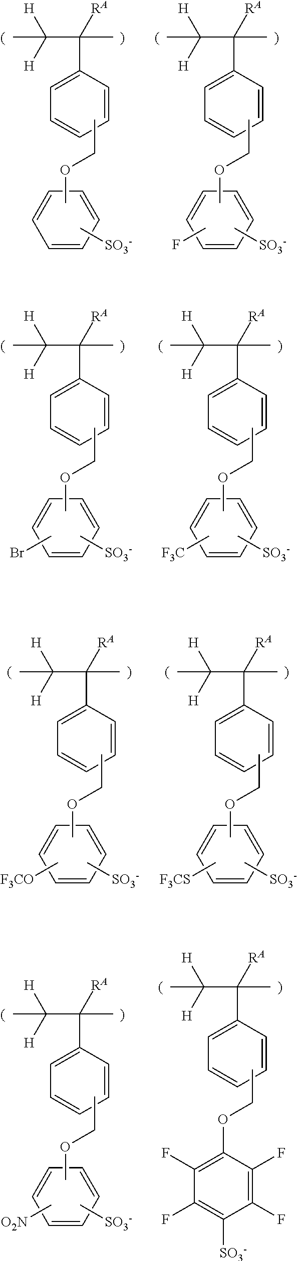

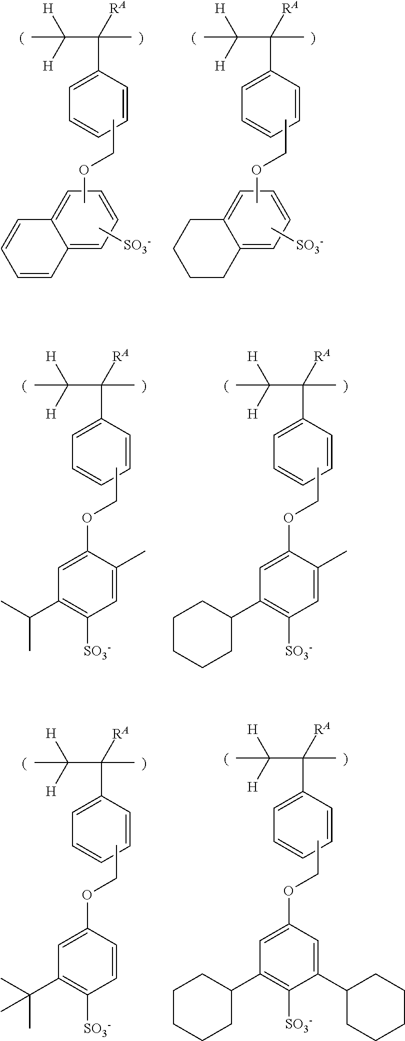

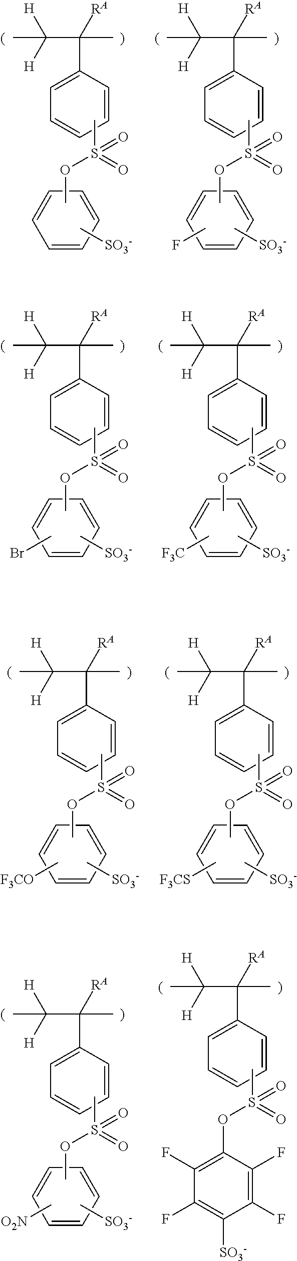

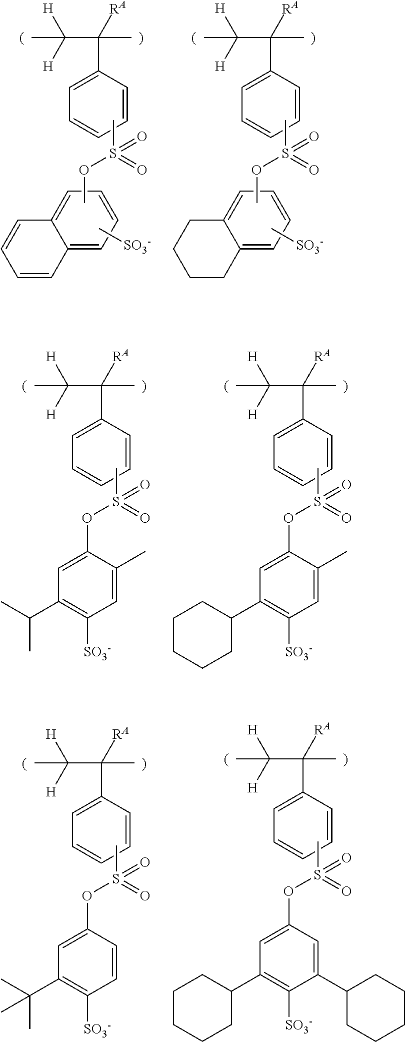

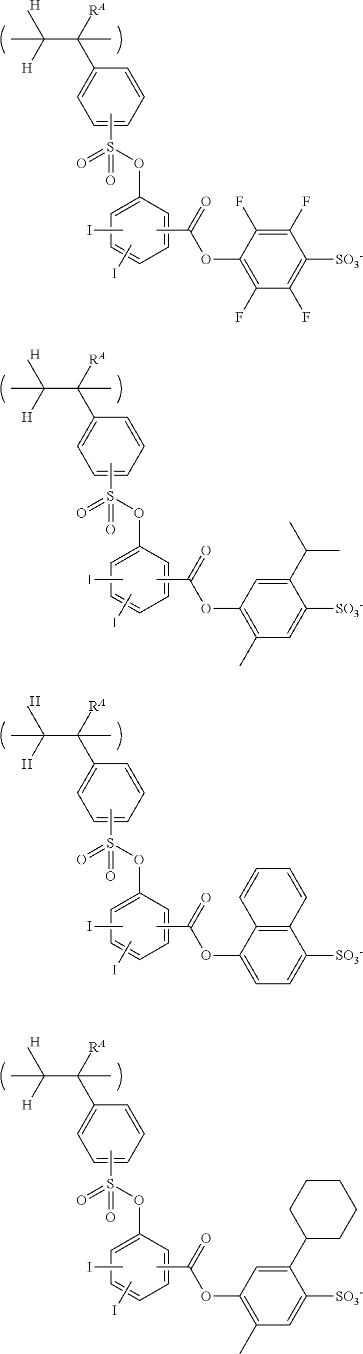

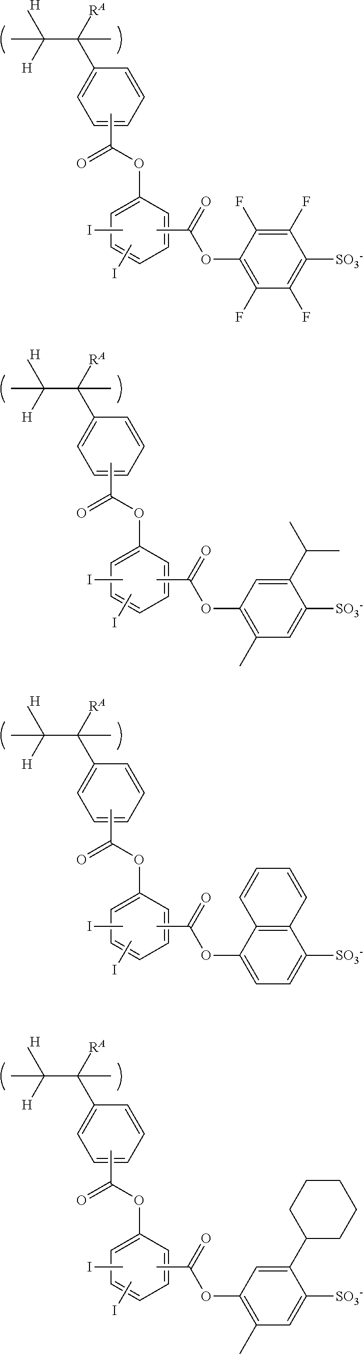

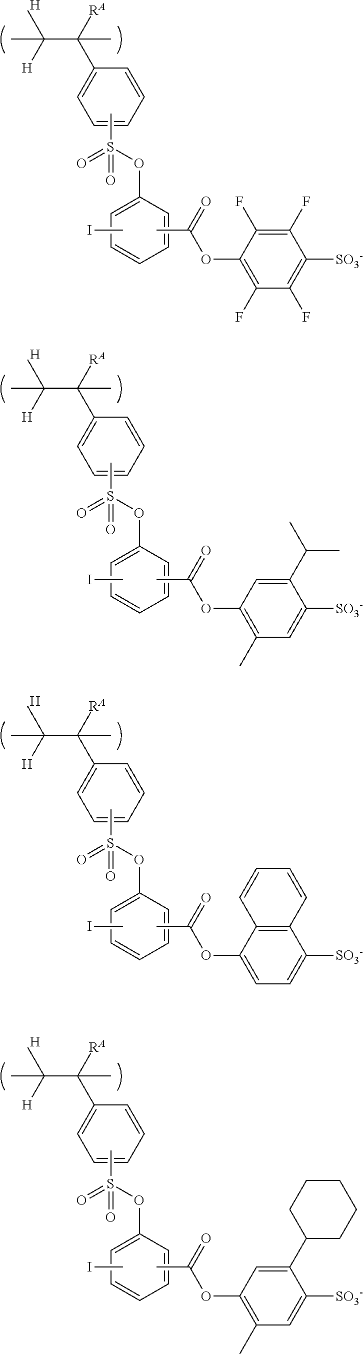

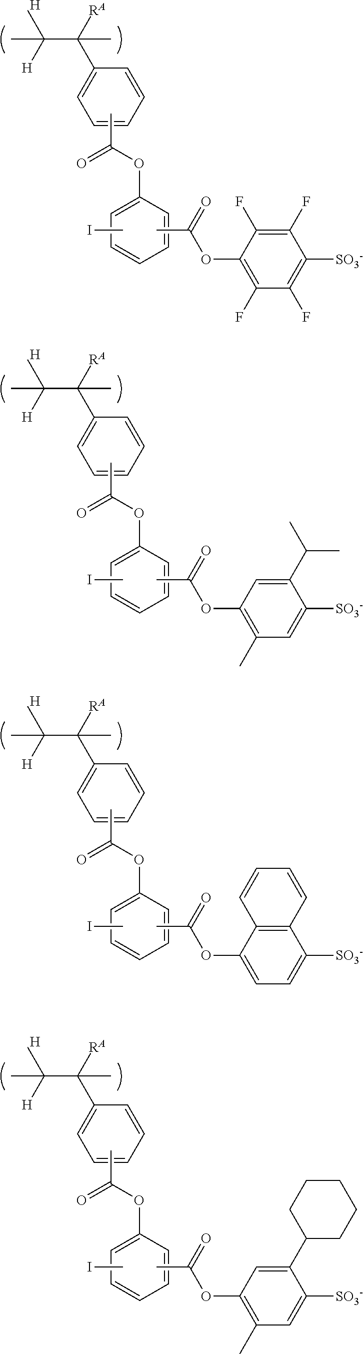

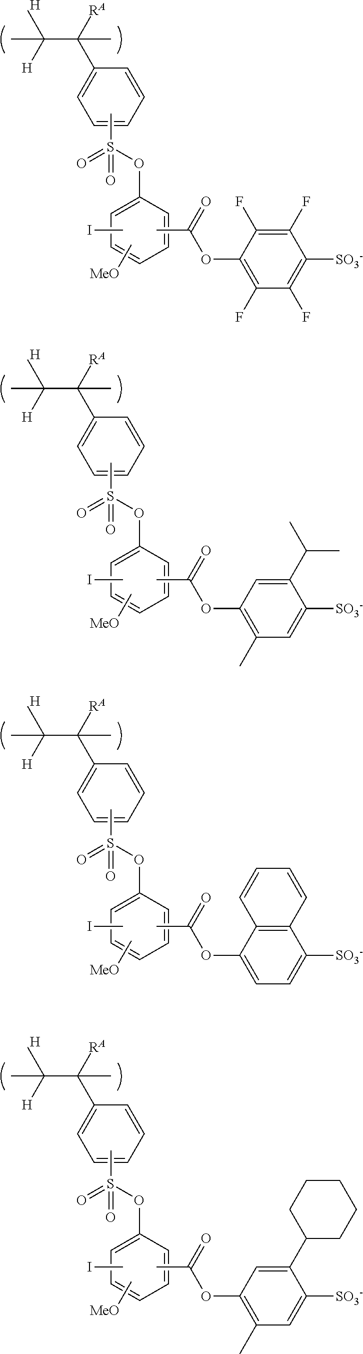

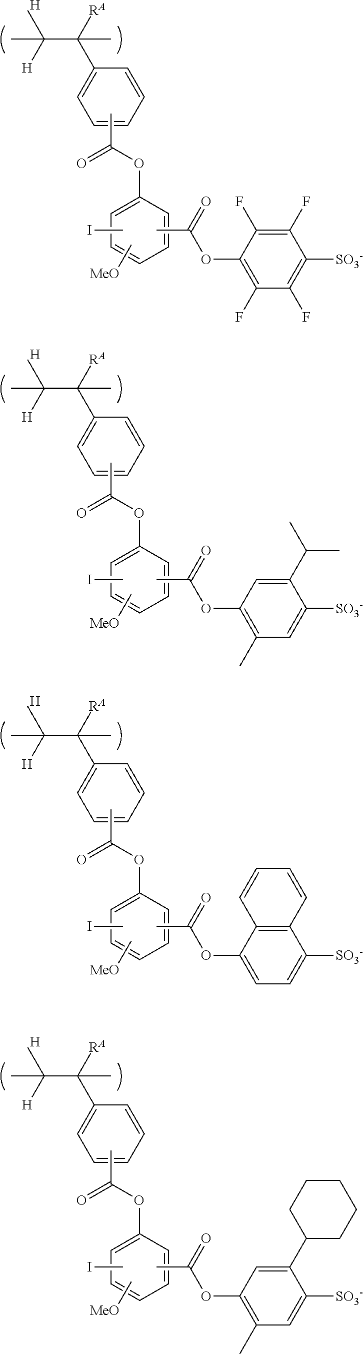

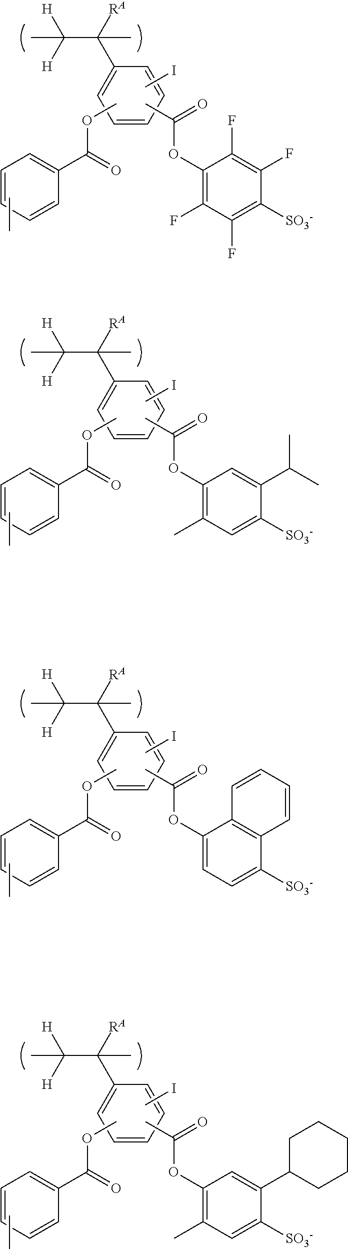

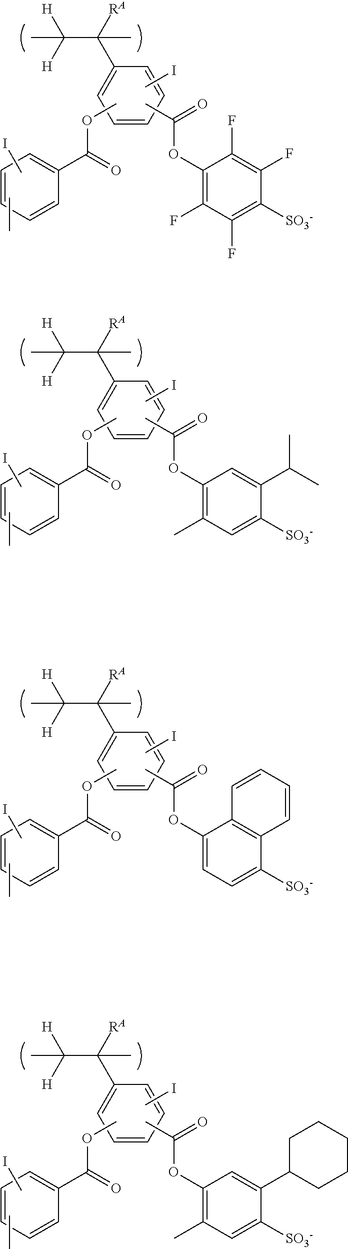

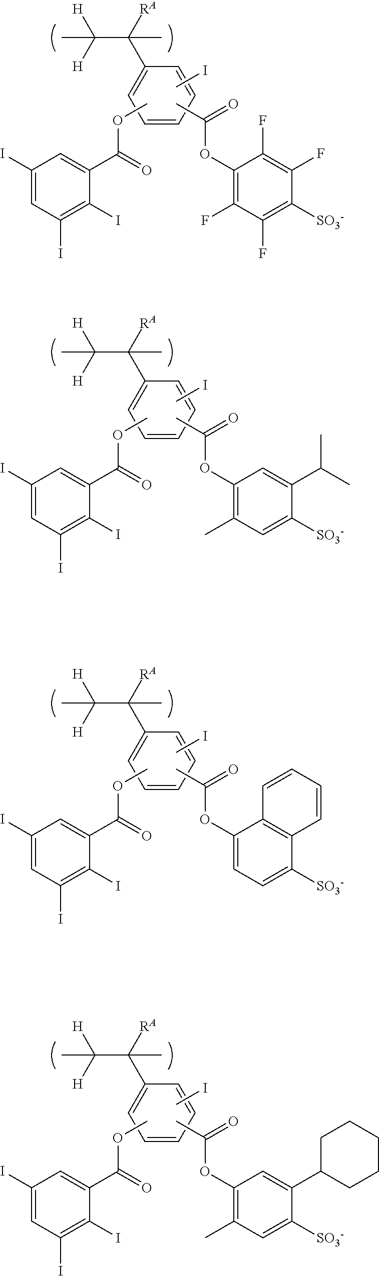

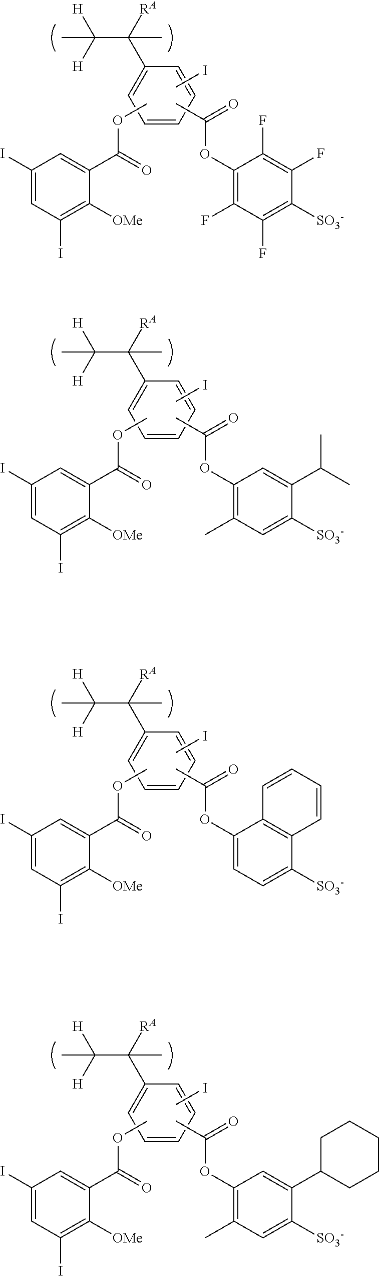

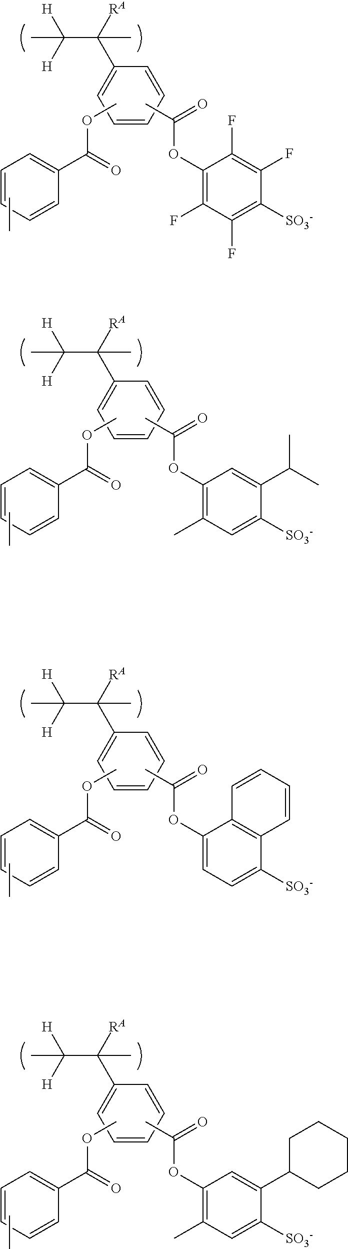

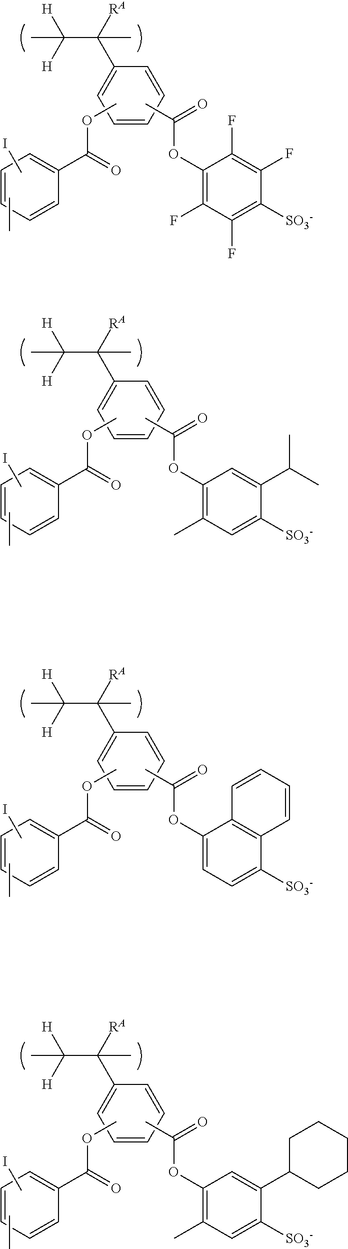

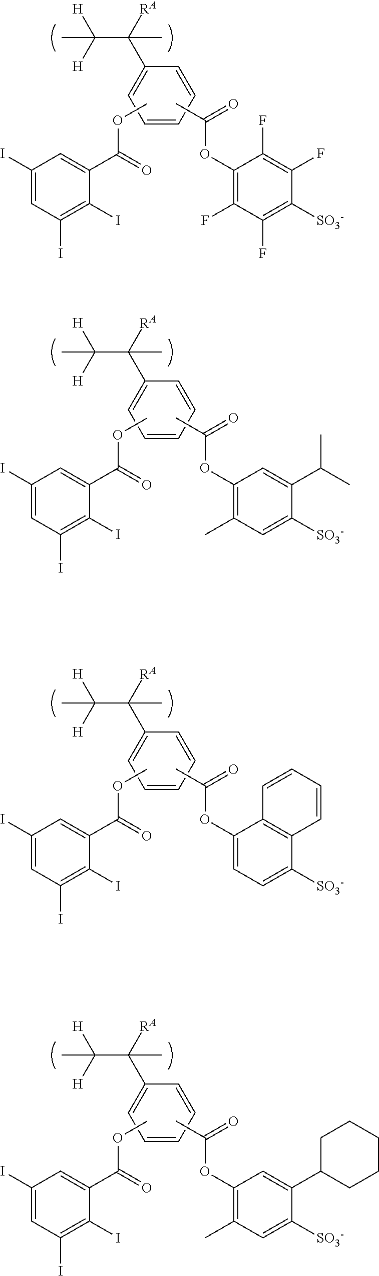

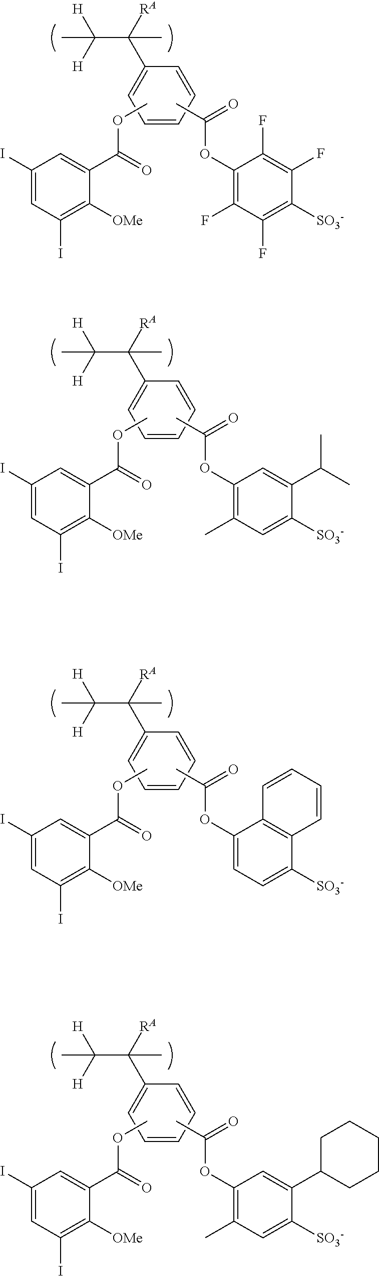

Specific examples of the anion of the repeating unit-c5 include, but are not limited to, those shown below. Note that, in the following formulae, R^{A} is as defined above.

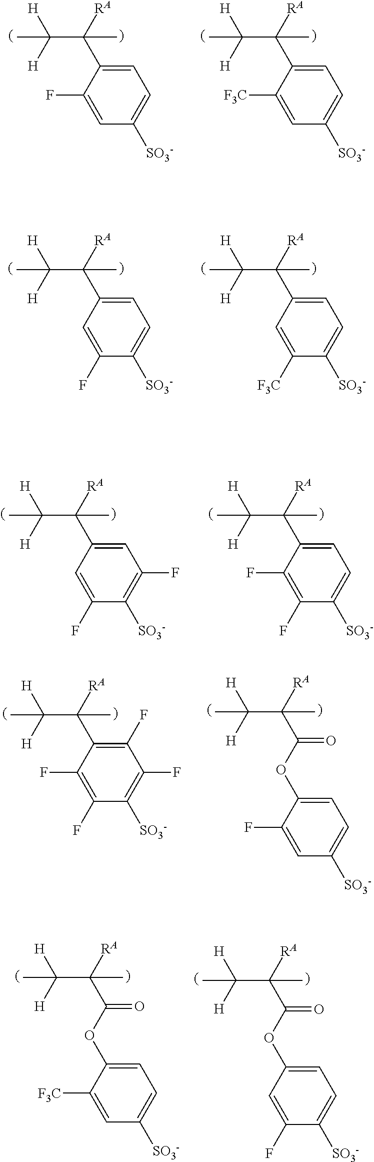

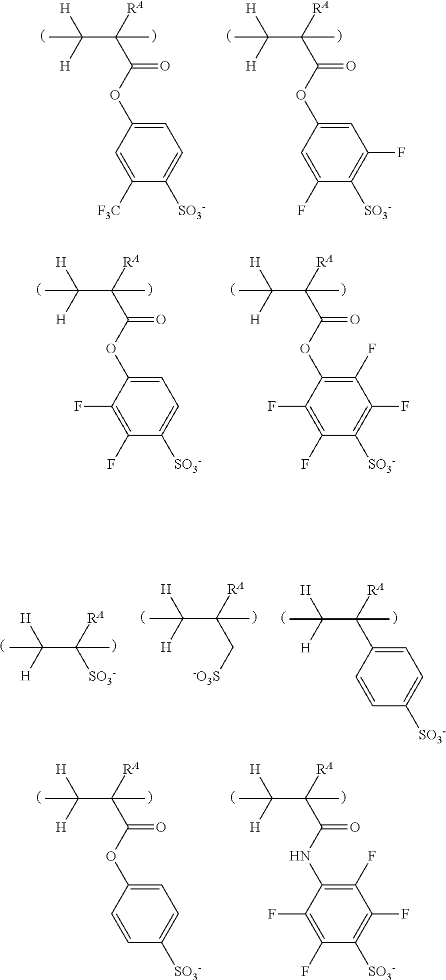

In the general formulae (c2) to (c5), A⁺ is an onium cation. Examples of the onium cation include a sulfonium cation, an iodonium cation, or an ammonium cation, etc., and a sulfonium cation or an iodonium cation is preferable. Specific examples of the sulfonium cation include those represented by the general formula (Z-1), those described in paragraphs to of JP2024-003744A, those described in paragraphs to of JP2023-169812A, and those represented by the general formula (Z-3), but are not limited thereto. Specific examples of the iodonium cation include those represented by the general formula (Z-2) and those described in paragraph of JP2024-000259A, but are not limited thereto. Specific examples of the ammonium cation include, but are not limited to, the same ones as those given as examples of the ammonium cation represented by the general formula (am-1) described later.

Specific structures of the repeating units “c1” to “c5” include any combination of the anions and cations described above.

Of the repeating units-c1 to c5, the repeating units-c2 to c5 are preferably from the viewpoint of suppressing acid diffusion, the repeating units-c2, c4, and c5 are more preferably from the viewpoint of the acid strength of the generated acid, and the repeating unit-c2 is further preferably from the viewpoint of solvent solubility.

The base polymer (B) may further contain a repeating unit having a structure in which a hydroxy group is protected by an acid-labile group (hereinafter, also referred to as repeating unit-d). The repeating unit-d is not particularly limited as long as it has one or more structures in which a hydroxy group is protected and the protecting groups are decomposed by the action of an acid to generate a hydroxy group, but is preferably represented by the following general formula (d1).

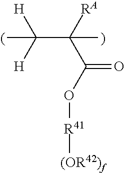

In the general formula (d1), R^{A} is as defined above. R⁴¹ is a hydrocarbon group having 1 to 30 carbon atoms and a valence of (f+1) and optionally containing a heteroatom. R⁴² is an acid-labile group. “f” is an integer from 1 to 4.

In the general formula (d1), the acid-labile group represented by R⁴² may be any group that can be deprotected by the action of an acid to generate a hydroxy group. The structure of R⁴² is not particularly limited, but is preferably an acetal structure, a ketal structure, an alkoxycarbonyl group, or an alkoxymethyl group represented by the following general formula (d2), etc. and, in particular, an alkoxymethyl group represented by the following general formula (d2) is preferable.

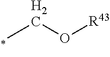

In the formula, “*” represents a bonding point. R⁴³ is a hydrocarbyl group having 1 to 15 carbon atoms.

Specific examples of the acid-labile group represented by R⁴², the alkoxymethyl group represented by the general formula (d2), and the repeating unit-d include the same ones as those given as examples in the description of the repeating unit-d described in JP2020-111564A.

The base polymer (B) may further contain a repeating unit-e derived from indene, benzofuran, benzothiophene, acenaphthylene, chromone, coumarin, norbornadiene, or a derivative thereof. Specific examples of monomers providing the repeating unit-e include, but are not limited to, those shown below.

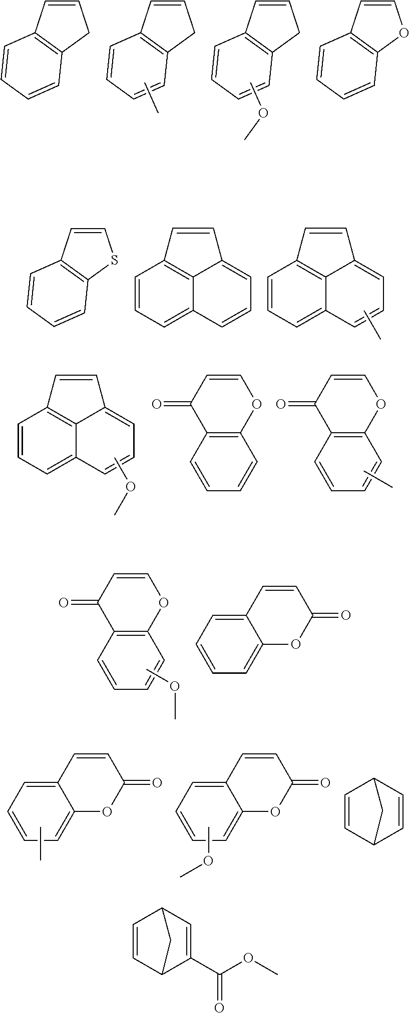

The base polymer (B) may further contain a repeating unit-f derived from indane, vinylpyridine, or vinylcarbazole.

In the base polymer of the present invention, the content ratios of the repeating units-a1, a2, a3, b1, b2, c1 to c5, d, e, and f are preferably 0≤a1≤0.8, 0≤a2≤0.8, 0≤a3≤0.6, 0≤b1≤0.6, 0≤b2≤0.6, 0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3, and more preferably 0≤a1≤0.7, 0≤a2≤0.7, 0≤a3≤0.5, 0≤b1≤0.5, 0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3. Provided that 0<a1+a2+a3≤1.0 and a1+a2+a3+b1+b2+c1+c2+c3+c4+d+e+f≤1.0.

The weight average molecular weight (Mw) of the base polymer is preferably 1,000 to 500,000, and more preferably 3,000 to 100,000. When the Mw is in this range, sufficient etching resistance is obtained, and there is no risk of a decrease in resolution due to an inability to ensure a difference in dissolution rate before and after exposure. In the present invention, the Mw is a polystyrene-equivalent measurement value obtained by gel permeation chromatography (GPC) using THF or N, N-dimethylformamide (DMF) as a solvent.

Furthermore, since the influence of Mw/Mn on the molecular weight distribution of the base polymer tends to become greater as the pattern rules become finer, Mw/Mn is preferably narrowly dispersed with a range of 1.0 to 2.0, in order to obtain a resist composition suitable for use with fine pattern dimensions. Within the above range, there are few low- or high-molecular-weight polymers compared with Mw, and there is no risk of foreign matter being found on the pattern or the shape of the pattern deteriorating after exposure.

To synthesize the base polymer, for example, a monomer that gives the repeating units described above may be polymerized by heating in an organic solvent with the addition of a radical polymerization initiator.

Specific examples of organic solvents used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of these initiators added is preferably 0.01 to 25 mol % per the total amount of monomers to be polymerized. The reaction temperature is preferably 50 to 150° C., and more preferably 60 to 100° C. The reaction time is preferably 2 to 24 hours, and from the viewpoint of production efficiency, more preferably 2 to 12 hours.

The polymerization initiator may be added to the monomer solution and then fed into the reaction vessel, or the polymerization initiator may be prepared separately from the monomer solution, and each of them may be fed independently into the reaction vessel. Since there is a possibility that the polymerization reaction may proceed during the waiting time and result in the formation of super-polymers due to radicals generated from the initiator, and also from the viewpoint of quality control, it is preferable to prepare the monomer solution and the initiator solution independently and add them dropwise. The acid-labile group may be used as is after being introduced into the monomer, or may be protected or partially protected after polymerization. In addition, known chain transfer agents such as dodecyl mercaptan and 2-mercaptoethanol may be used in combination to adjust the molecular weight. In this case, the amount of these chain transfer agents added is preferably 0.01 to 20 mol % per the total amount of monomers to be polymerized.

In the case of monomers containing hydroxy groups, the hydroxy groups may be substituted with acetal groups such as ethoxyethoxy groups, which are easily deprotected by acid, during polymerization, and then deprotected with weak acid and water after polymerization, or the hydroxy groups may be substituted with acetyl groups, formyl groups, pivaloyl groups, etc., and then hydrolyzed with an alkali after polymerization.

When copolymerizing hydroxystyrene or hydroxyvinylnaphthalene, hydroxystyrene or hydroxyvinylnaphthalene and other monomers may be polymerized by heating in an organic solvent with the addition of a radical polymerization initiator, but alternatively, acetoxystyrene or acetoxyvinylnaphthalene can be used, and after polymerization, the acetoxy group may be deprotected by alkaline hydrolysis to produce polyhydroxystyrene or hydroxypolyvinylnaphthalene.

Ammonia water, triethylamine, etc. can be used as the base for alkaline hydrolysis. In addition, the reaction temperature is preferably-20 to 100° C., and more preferably 0 to 60° C. The reaction time is preferably 0.2 to 100 hours, and more preferably 0.5 to 20 hours.

The amount of each monomer in the monomer solution may be appropriately set so as to obtain the preferred content ratio of the repeating units described above.

For the base polymer obtained by the above-mentioned manufacturing method, a reaction solution obtained through a polymerization reaction may be handled as the final product, or a purification process such as a reprecipitation method in which the polymerization solution is added to a poor solvent to obtain a powder may be performed, and then the resulting powder may be handled as the final product. However, from the viewpoint of work efficiency and quality stabilization, it is preferable to dissolve the powder obtained through the purification process in a solvent and handle the base polymer solution thus obtained as the final product.

Specific examples of the solvent used in this case include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high-boiling-point alcohol solvents such as diethylene glycol, propylene glycol, glycerin, 1,4-butanediol, and 1,3-butanediol; and mixed solvents thereof as described in paragraphs to of JP2008-111103A.

The concentration of the polymer in the base polymer solution is preferably 0.01 to 30% by mass, and more preferably 0.1 to 20% by mass.

It is preferable to filter the reaction solution or the base polymer solution. Filtering can remove foreign matter and gels that may cause defects, which is effective for stabilizing quality.

The material of the filter used in the filter filtration may be a fluorocarbon-based, cellulose-based, nylon-based, polyester-based, or hydrocarbon-based material, but in the filtration process of the resist composition, a filter made of a fluorocarbon-based material known as Teflon (registered trademark), a hydrocarbon-based material such as polyethylene or polypropylene, or nylon is preferred. The pore size of the filter can be appropriately selected according to the target degree of cleanliness, but is preferably 100 nm or less, and more preferably 20 nm or less. In addition, these filters may be used alone or in combination. For the filtration method, the solution may be passed through just the filter once, but it is more preferable to circulate the solution and perform filtration a plurality of times. The filtration process may be performed in any order and any number of times in the polymer production process, but it is preferable to filter the reaction solution after the polymerization reaction, the polymer solution, or both.

The base polymer (B) may be used alone or in a combination of two or more different polymers having different composition ratios, Mw, and/or Mw/Mn. In addition to the above-mentioned polymers, the base polymer (B) may also contain a hydrogenated ring-opening metathesis polymer, and in this regard, the polymers described in JP2003-066612A can be used.

In addition, the chemically amplified resist composition of the present invention preferably further contains at least one constituent selected from an organic solvent, a quencher, a photo-acid generator other than the above-mentioned photo-acid generator, and a surfactant. Each component is explained in detail below.

### [Organic Solvent (C)]

The chemically amplified resist composition of the present invention may contain an organic solvent as component (C). The organic solvent (C) is not particularly limited as long as it is capable of dissolving each component described above and below. Specific examples of such organic solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and mixed solvents thereof.

Among these organic solvents, 1-ethoxy-2-propanol, PGMEA, cyclohexanone, GBL, DAA, and mixed solvents thereof are preferable, as they have particularly excellent solubility for the base polymer for component (B).

In the chemically amplified resist composition of the present invention, the content of the organic solvent (C) is preferably 200 to 5,000 parts by mass, and more preferably 400 to 3,500 parts by mass, per 80 parts by mass of the base polymer (B). The organic solvent (C) may be used alone or in a combination of two or more types.

### [Quencher (D)]

The chemically amplified resist composition of the present invention may contain a quencher as component (D). In the present invention, a quencher means a material that traps the acid generated by the photo-acid generator in the chemically amplified resist composition, thereby preventing the acid from diffusing into unexposed areas, and enabling a desired pattern to be formed.

The quencher (D) includes an onium salt represented by the following general formula (2) or (3).

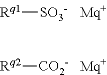

In the general formula (2), R^{q1} is a hydrogen atom, or a hydrocarbyl group having 1 to 40 carbon atoms and optionally containing a heteroatom, except for ones in which the hydrogen atom bonded to the carbon atom at the α-position of the sulfo group is substituted with a fluorine atom or a fluoroalkyl group. In the general formula (3), R^{q2} is a hydrogen atom, or a hydrocarbyl group having 1 to 40 carbon atoms and optionally containing a heteroatom.

Specific examples of the hydrocarbyl group having 1 to 40 carbon atoms represented by Rol include alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbuthyl group, a norbornyl group, a tricyclo[5.2.1.0^{2,6}]decyl group, and an adamantyl group; and aryl groups having 6 to 40 carbon atoms, such as a phenyl group, a naphthyl group, and an anthracenyl group. In addition, some or all of the hydrogen atoms of the hydrocarbyl group may be substituted with a group containing a heteroatom such as an oxygen atom, a sulfur atom, a nitrogen atom, or a halogen atom, and some the —CH₂— of the hydrocarbyl group may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the hydrocarbyl group represented by R^{q2} include, in addition to the substituents given as specific examples of R^{q1}, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt represented by the general formula (2) include, but are not limited to, those shown below.

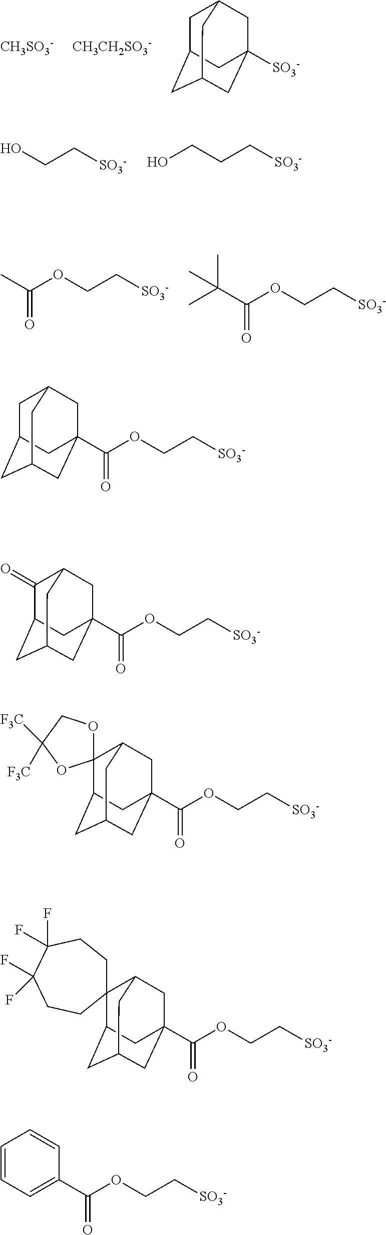

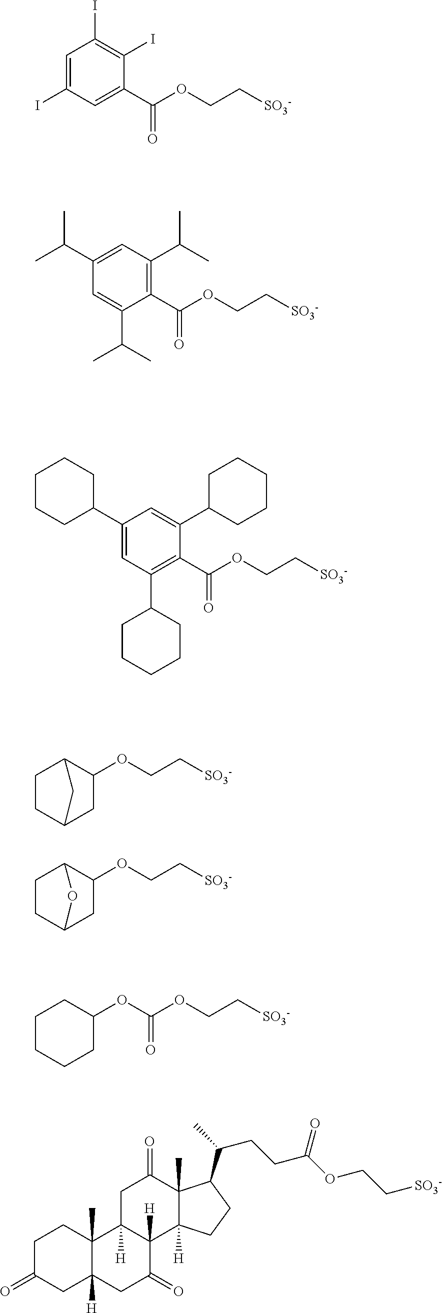

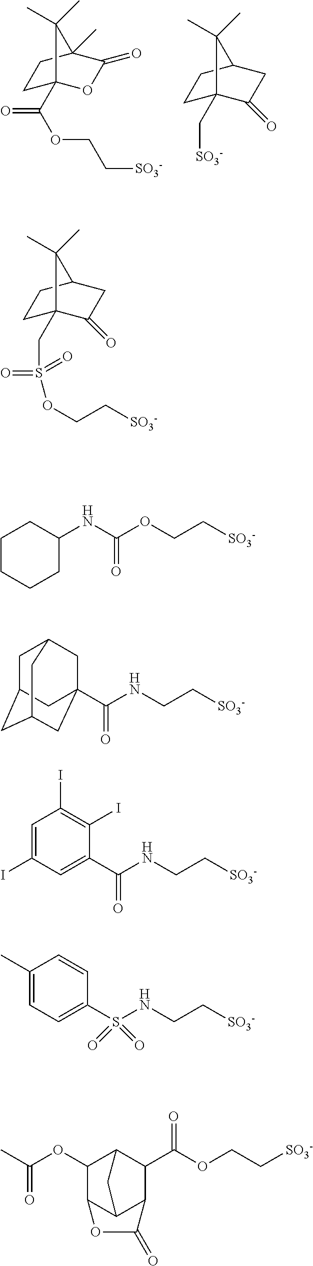

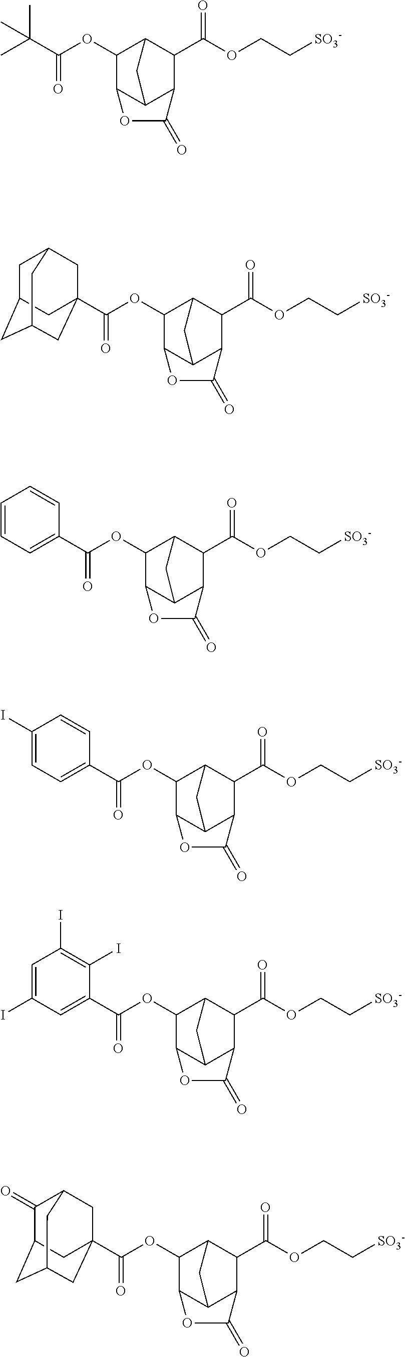

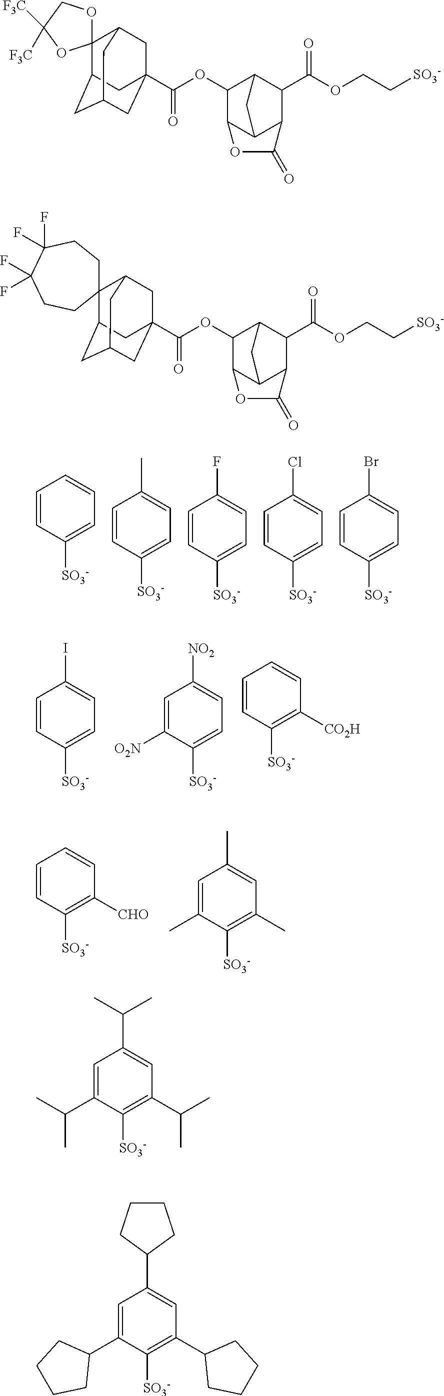

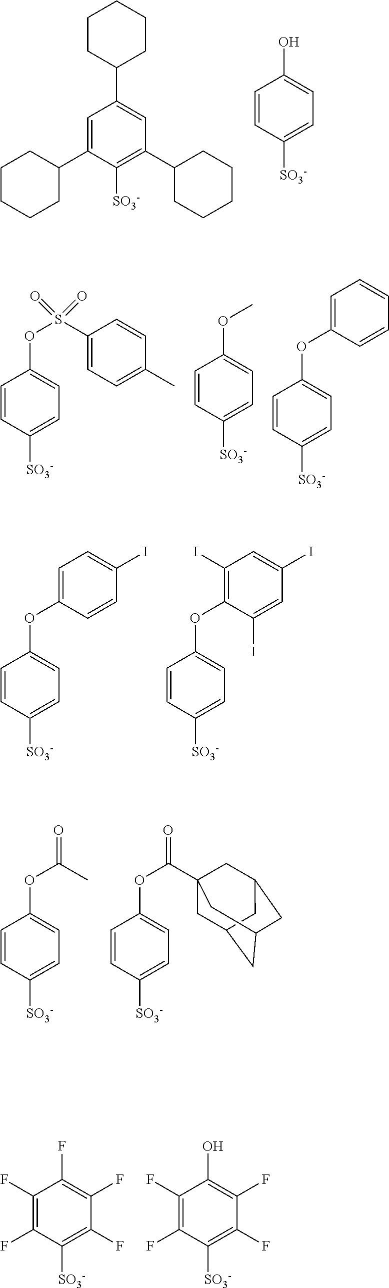

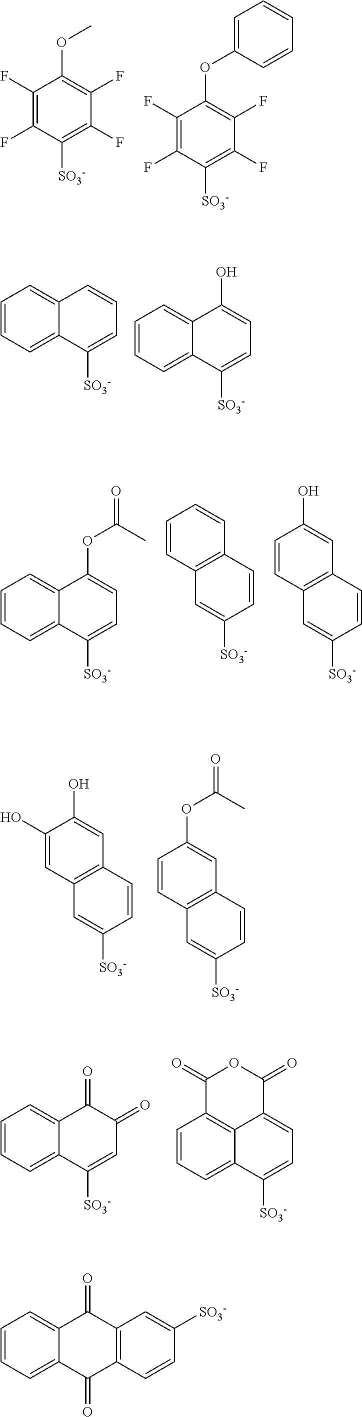

Specific examples of the anion of the onium salt represented by the general formula (3) include, but are not limited to, those shown below.

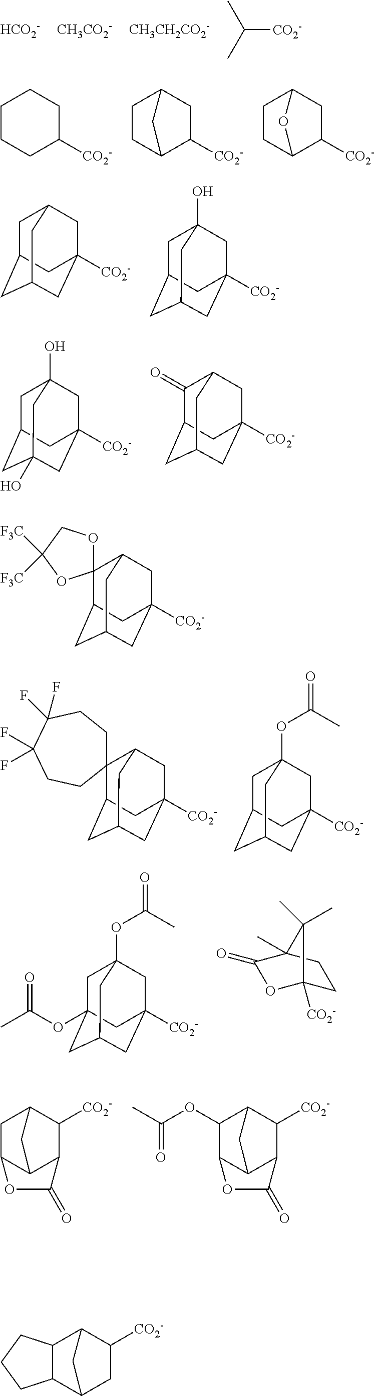

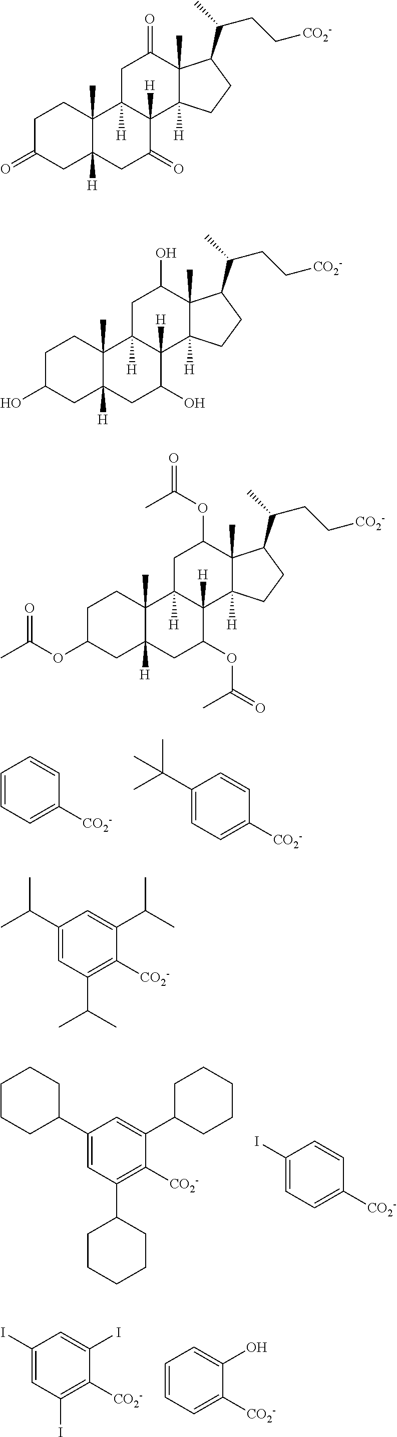

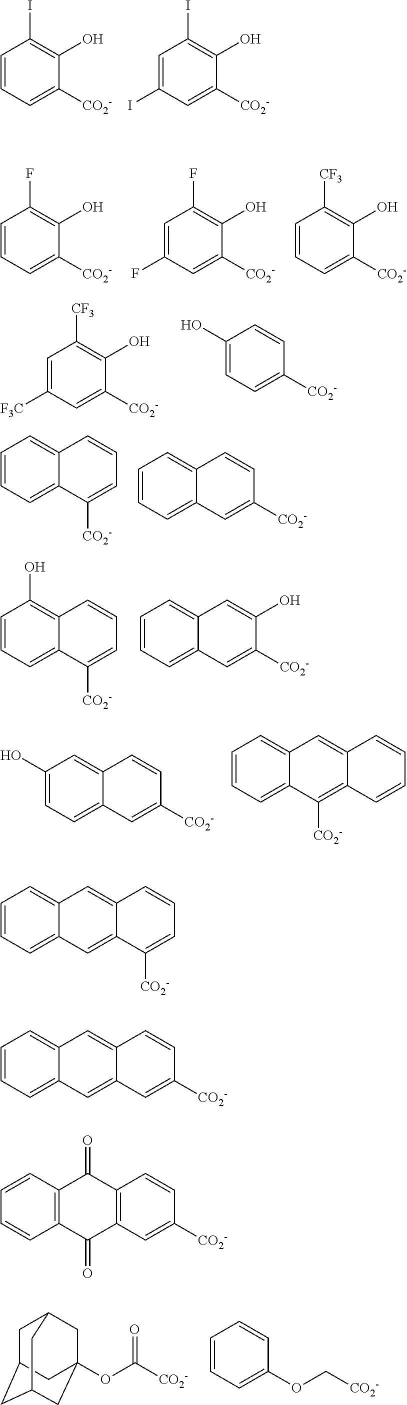

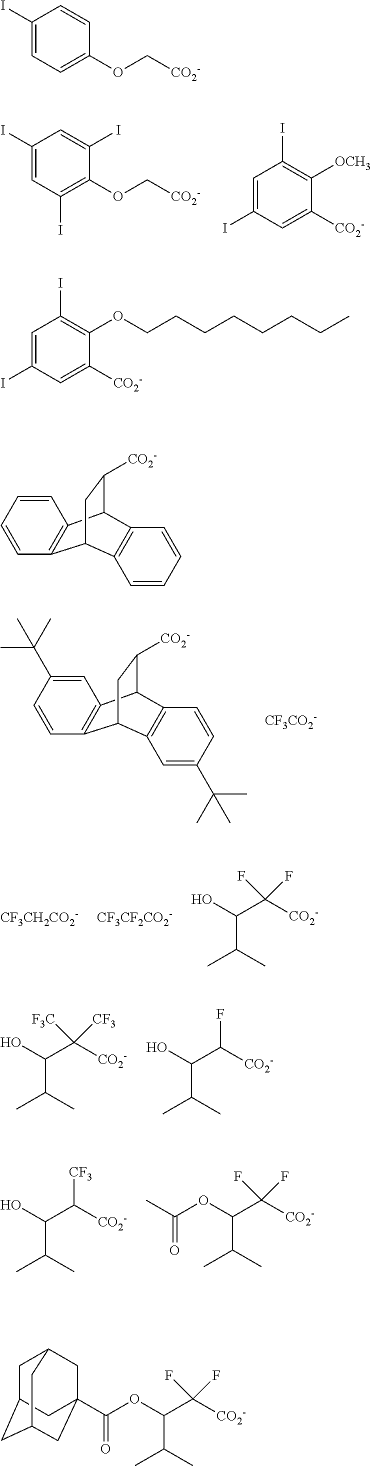

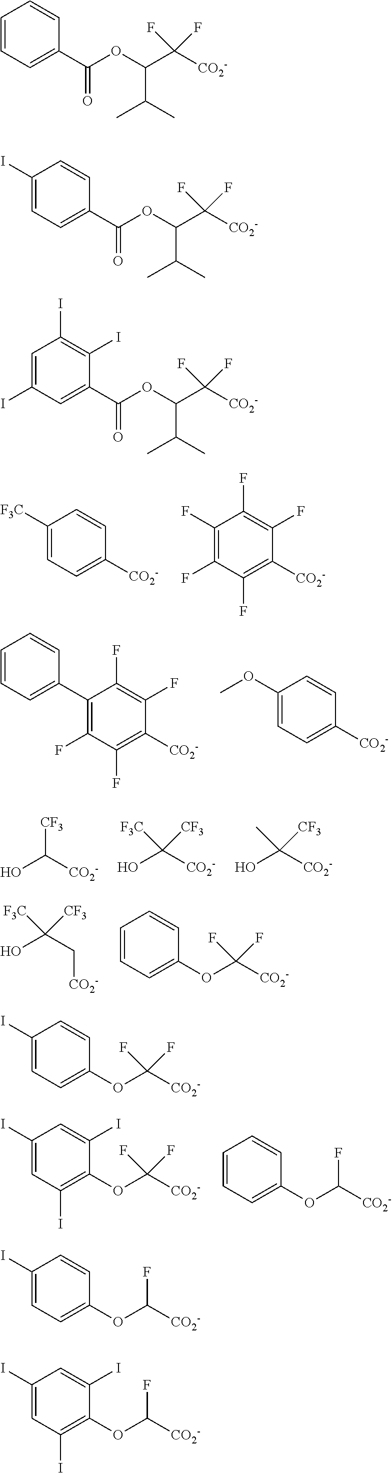

In the general formulae (2) and (3), M^{q+} is an onium cation. Examples of the onium cation include a sulfonium cation, an iodonium cation, an ammonium cation, etc. Specific examples of the sulfonium cation and iodonium cation include, but are not limited to, those represented by Z⁺ and given as specific examples of the sulfonium cation and iodonium cation in the explanation of the general formula (1). Specific examples of the ammonium cation include those represented by the following general formula (am-1).

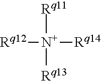

In the general formula (am-1), R^{q11} to R^{q14} are each independently a hydrocarbyl group having 1 to 40 carbon atoms and optionally containing a heteroatom. In addition, R^{q11} and R^{q12} may be bonded to each other to form a ring together with the nitrogen atom to which they are bonded. Specific examples of the hydrocarbyl group include the same ones as those given as examples of the hydrocarbyl group represented by R¹ in the explanation of the general formula (1).

Specific examples of the ammonium cation represented by the general formula (am-1) include, but are not limited to, those shown below.

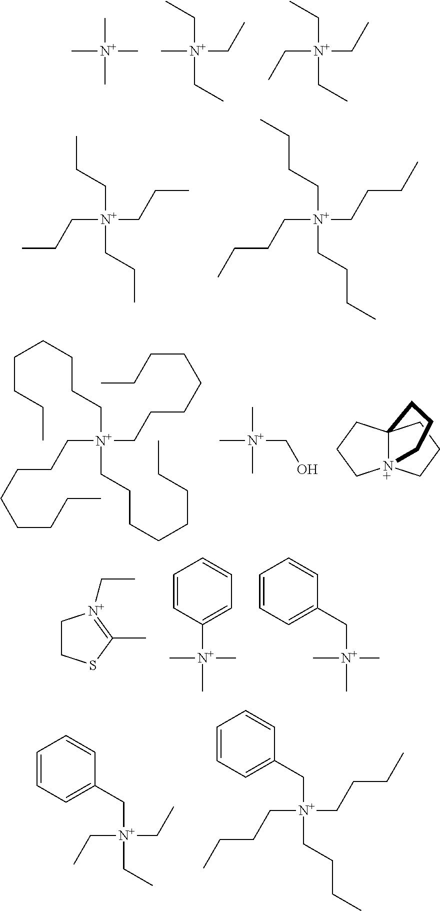

Specific examples of the onium salt represented by the general formula (2) or (3) include any combination of the above-mentioned anions and cations. These onium salts can be easily prepared by ion exchange reactions using known organic chemical methods. For ion exchange reactions, for example, JP2007-145797A can be referred to.

The onium salt represented by the general formula (2) or (3) acts as a quencher in the chemically amplified resist composition of the present invention. This is because each counter anion of the onium salt is a conjugate base of a weak acid. The term “weak acid” used herein means an acidity that is not capable of deprotecting the acid-labile group of the acid-labile-group-containing unit used in the base polymer. The onium salt represented by the general formula (2) or (3) functions as a quencher when used in combination with an onium salt type photo-acid generator having as a counter anion a conjugate base of a strong acid such as a sulfonic acid whose α-position is fluorinated. That is, When an onium salt that generates a strong acid such as a sulfonic acid whose α-position is fluorinated and an onium salt that generates a weak acid such as a non-fluorinated sulfonic acid or carboxylic acid are mixed and used, then when the strong acid generated from the photo-acid generator by irradiation with a high-energy beam collides with an onium salt having an unreacted weak acid anion, the weak acid is released by salt exchange to generate an onium salt having a strong acid anion. In this process, the strong acid is exchanged for a weaker acid with lower catalytic activity, which appears to deactivate the acid and enables suppression of acid diffusion.

In addition, as the quencher (D), onium salts having a sulfonium cation and a phenoxide anion moiety in the same molecule as described in JP6848776B2, onium salts having a sulfonium cation and a carboxylate anion moiety in the same molecule as described in JP6583136B2 and JP2020-200311A, and onium salts having an iodonium cation and a carboxylate anion moiety in the same molecule as described in JP6274755B2 can also be used.

Here, when the photo-acid generator that generates a strong acid is an onium salt, then as described above, the strong acid generated by irradiation with high-energy beams can be exchanged for a weak acid; on the other hand, it is thought that the weak acid generated by irradiation with high-energy beams collides with the onium salt that generates the unreacted strong acid, and is unlikely to undergo salt exchange. This is due to the phenomenon that the onium cation is more likely to form an ion pair with the anion of the strong acid.

When the chemically amplified resist composition of the present invention contains an onium salt represented by general formula (2) or (3) as the quencher (D), the content thereof is preferably 0.1 to 20 parts by mass, and more preferably 0.1 to 10 parts by mass, per 80 parts by mass of the base polymer (B). When the onium salt type quencher for component (D) is within the above range, the resolution is good and there is no significant decrease in sensitivity, so that is preferable. The onium salt represented by the general formula (2) or (3) can be used alone or in a combination of two or more types.

The chemically amplified resist composition of the present invention may contain a nitrogen-containing compound as a quencher (D). Examples of the nitrogen-containing compound for component (D) include primary, secondary, or tertiary amine compounds described in paragraphs to of JP2008-111103A, particularly, amine compounds having a hydroxy group, an ether bond, an ester bond, a lactone ring, a cyano group, or a sulfonate ester bond. In addition, examples include compounds in which a primary or secondary amine is protected with a carbamate group, such as the compounds described in JP3790649B2.

In addition, a sulfonium salt of a sulfonic acid having a nitrogen-containing substituent may be used as the nitrogen-containing compound. Such a compound functions as a quencher in the unexposed areas and loses its quenching ability by neutralization with the acid generated by itself in the exposed areas, functioning as a so-called photodegradable base. By using a photodegradable base, the contrast between the exposed areas and the unexposed areas can be further enhanced. For example, JP2009-109595A and JP2012-046501A can be referred to for photodegradable bases.

When the chemically amplified resist composition of the present invention contains a nitrogen-containing compound as a quencher (D), the content thereof is preferably 0.001 to 12 parts by mass, and more preferably 0.01 to 8 parts by mass, per 80 parts by mass of the base polymer (B). The nitrogen-containing compound may be used alone or in a combination of two or more types.

### [Other Photo-Acid Generators (E)]

The chemically amplified resist composition of the present invention further may contain a photo-acid generator other than the component (A)(hereinafter, also referred to as other photo-acid generators) as component (E). The other photo-acid generators are not particularly limited as long as they are compounds that generate an acid upon irradiation with high-energy beams. Suitable other photo-acid generators include those represented by the following general formula (4) or (5).

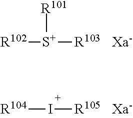

In the general formulae (4) and (5), R¹⁰¹ to R¹⁰⁵ are each independently a hydrocarbyl group having 1 to 20 carbon atoms and optionally containing a heteroatom. In addition, any two of R¹⁰¹, R¹⁰², and R¹⁰³ may be bonded to each other to form a ring together with the sulfur atom to which they are bonded.

Specific examples of the cation of the sulfonium salt represented by the general formula (4) and the cation of the iodonium salt represented by the general formula (5) include the same ones as those given as specific examples of the sulfonium cation and the iodonium salt represented by Z⁺ in the explanation of the general formula (1), but are not limited thereto.

In the above general formulae (4) and (5), Xa⁻ is an anion of a strong acid. Examples of the anion of the strong acid include those represented by any of the general formulae (c1-1) to (c1-5).

As other photo-acid generators for component (E), compounds represented by the following general formula (6) are also preferable.

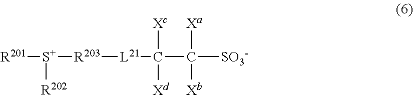

In the general formula (6), R²⁰¹ and R²⁰² are each independently a hydrocarbyl group having 1 to 30 carbon atoms and optionally containing a heteroatom. R²⁰³ is a hydrocarbylene group having 1 to 30 carbon atoms and optionally containing a heteroatom. In addition, any two of R²⁰¹, R²⁰², and R²⁰³ may be bonded to each other to form a ring together with the sulfur atom to which they are bonded.

The hydrocarbyl group having 1 to 30 carbon atoms and represented by R²⁰¹ and R²⁰² may be saturated or unsaturated, and may be linear, branched, or cyclic. Specific examples thereof include alkyl groups having 1 to 30 carbon atoms, such as a methyl group, an ethyl group, an n-propyl group, an isopropyl group, an n-butyl group, an isobutyl group, a sec-butyl group, a tert-butyl group, a tert-pentyl group, an n-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups obtained by combining these. In addition, some or all of the hydrogen atoms of the hydrocarbyl group may be substituted with a group containing a heteroatom such as an oxygen atom, a sulfur atom, a nitrogen atom, or a halogen atom, and some of the —CH₂— of the hydrocarbyl group may be substituted with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

The hydrocarbylene group having 1 to 30 carbon atoms and represented by R²⁰³ may be saturated or unsaturated, and may be linear, branched, or cyclic. Specific examples thereof include alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentadiyl group, a cyclohexanediyl group, a norbornanediyl group, and an adamantanediyl group; and arylene groups, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group. In addition, some or all of the hydrogen atoms of the hydrocarbylene group may be substituted with a group containing a heteroatom such as an oxygen atom, a sulfur atom, a nitrogen atom, or a halogen atom, and some of the —CH₂— of the hydrocarbylene group may be substituted with a group containing a heteroatom such as an oxygen atom, a sulfur atom or a nitrogen atom, and as a result, the hydrocarbylene group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. As the heteroatom, an oxygen atom is preferable.

In the general formula (6), L²¹ is a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include the same ones as those given as examples of the hydrocarbylene group represented by R²⁰³.

In the general formula (6), X^{a}, X^{b}, X^{c}, and X^{d} each independently represent a hydrogen atom, a fluorine atom, or a trifluoromethyl group. Provided that at least one of X^{a}, X^{b}, X^{c}, and X^{d} is a fluorine atom or a trifluoromethyl group.

The photo-acid generator represented by the general formula (6) is preferably one represented by the following general formula (6′).

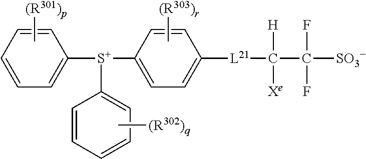

In the general formula (6′), L²¹ is as defined above. Xe is a hydrogen atom or a trifluoromethyl group, and preferably a trifluoromethyl group. R³⁰¹, R³⁰², and R³⁰³ are each independently a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (c1-1-1). “p” and “q” each independently represent an integer from 0 to 5, and “r” represents an integer from 0 to 4.

Examples of photo-acid generators represented by the general formula (6) include the same ones as those given as examples of photo-acid generators represented by formula (2) in JP2017-026980A.

Among the other photo-acid generators, those containing anions represented by the general formula (c1-1-1) or (c1-4) are particularly preferrable because they have low acid diffusion and excellent solubility in solvents. In addition, those represented by the general formula (6′) are particularly preferable because they have extremely low acid diffusion.

When the chemically amplified resist composition of the present invention contains other photo-acid generators (E), the content thereof is preferably 0.1 to 40 parts by mass, and more preferably 0.5 to 20 parts by mass, per 80 parts by mass of base polymer (B). When the amount of the photo-acid generators (E) added is within the above range, the resolution is good and there is no risk of problems with foreign matter occurring after development or during stripping of the resist film, so that is preferable. The other photo-acid generators (E) may be used alone or in a combination of two or more types.

### [Surfactant (F)]

The chemically amplified resist composition of the present invention may further contain a surfactant as component (F). The surfactant (F) is preferably a surfactant that is insoluble or poorly soluble in water and soluble in an alkaline developer, or a surfactant that is insoluble or poorly soluble in water and an alkaline developer. Examples of such surfactants include those described in JP2010-215608A and JP2011-016746A.

Among the surfactants described in the above publications, preferable surfactants that are insoluble or poorly soluble in water and alkaline developers include FC-4430 (manufactured by 3M), Surflon (registered trademark) S-381 (manufactured by AGC Seimi Chemical Co., Ltd.), Olfine (registered trademark) E1004 (manufactured by Nissin Chemical Industry Co., Ltd.), KH-20 and KH-30 (manufactured by AGC Seimi Chemical Co., Ltd.), and oxetane ring-opening polymers represented by the following general formula (surf-1).

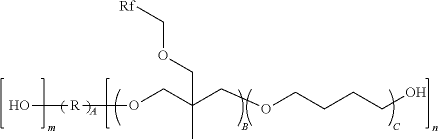

Here, R, Rf, A, B, C, “m”, and “n” apply only to the general formula (surf-1), regardless of the above descriptions. R is a divalent to tetravalent aliphatic group having 2 to 5 carbon atoms. Examples of the aliphatic group include divalent groups such as an ethylene group, a 1,4-butylene group, a 1,2-propylene group, a 2,2-dimethyl-1,3-propylene group, and a 1,5-pentylene group, and examples of trivalent or tetravalent groups include the following.

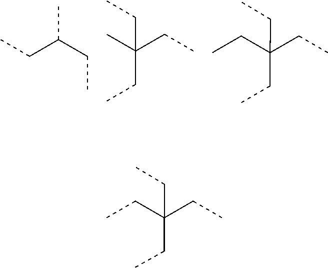

In the formulae, the dashed lines represent bonding points and are partial structures derived from glycerol, trimethylolethane, trimethylolpropane, and pentaerythritol, respectively.

Among these, 1,4-butylene group, 2,2-dimethyl-1,3-propylene group, etc. are preferred.

Rf is a trifluoromethyl group or a pentafluoroethyl group, and preferably a trifluoromethyl group. “m” is an integer from 0 to 3, “n” is an integer from 1 to 4, and the sum of “n” and “m” is the valence of R, and is an integer from 2 to 4. A is 1. B is an integer from 2 to 25, and preferably an integer from 4 to 20. C is an integer from 0 to 10, and preferably 0 or 1. In addition, the order of the constituent units in the general formula (surf-1) is not specified, and they may be bonded in blocks or randomly. The production of partially fluorinated oxetane ring-opening polymer surfactants is described in detail in the specifications of U.S. Pat. No. 5,650,483A, etc.

When no resist protective film is used in ArF immersion lithography, surfactants that are insoluble or poorly soluble in water and soluble in alkaline developers have a function of reducing water penetration and leaching by orienting on the surface of the resist film. Therefore, they are useful for suppressing the elution of water-soluble components from the resist film and reducing damage to the exposure device, and are also useful because they are soluble during development with an alkaline aqueous solution after exposure or post-exposure bake (PEB), making them less likely to become foreign matter that causes defects. Such surfactants are insoluble or poorly soluble in water and soluble in alkaline developers, are polymer type surfactants, are also known as hydrophobic resins, and are particularly preferable for their high water repellency and improved water slippage.

Specific examples of such polymeric surfactants include those containing at least one repeating unit selected from those represented by any one of the following general formulae (7A) to (7E).

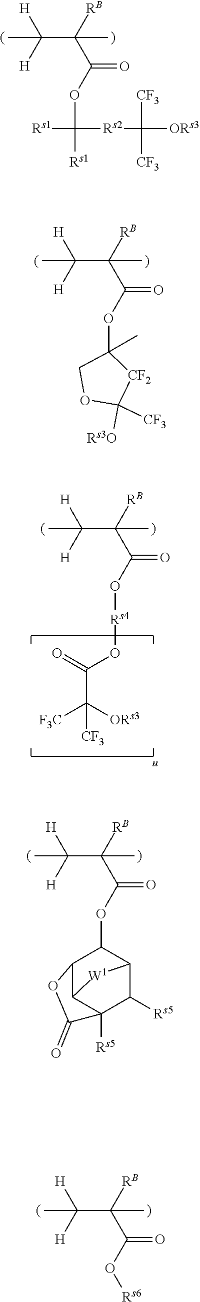

In the general formulae (7A) to (7E), RB is a hydrogen atom, a fluorine atom, a methyl group, or a trifluoromethyl group. W¹ is —CH₂—, —CH₂CH₂—, —O—, or two —H groups separated from each other. R^{s1} is each independently a hydrogen atom, or a hydrocarbyl group having 1 to 10 carbon atoms. R^{s2} is a single bond, or a linear or branched hydrocarbylene group having 1 to 5 carbon atoms. R^{s3} is each independently a hydrogen atom, a hydrocarbyl group or a fluorinated hydrocarbyl group having 1 to 15 carbon atoms, or an acid-labile group. When R^{s3} is a hydrocarbyl group or a fluorinated hydrocarbyl group, an ether bond or a carbonyl group may be present between the carbon-carbon bonds. R^{s4} is a (u+1)-valent hydrocarbon group or a fluorinated hydrocarbon group having 1 to 20 carbon atoms. “u” is 1, 2 or 3. R^{s5} is each independently a hydrogen atom, or a group represented by —C(═O)—O—R^{sa}. R^{sa} is a fluorinated hydrocarbyl group having 1 to 20 carbon atoms. R^{s6} is a hydrocarbyl group or a fluorinated hydrocarbyl group having 1 to 15 carbon atoms, and an ether bond or a carbonyl group may be present between the carbon-carbon bonds.

The hydrocarbyl group having 1 to 10 carbon atoms and represented by R^{s1} is preferably a saturated hydrocarbyl group, and may be linear, branched, or cyclic. Specific examples thereof include alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, and an n-decyl group; and cyclic saturated hydrocarbyl groups having 3 to 10 carbon atoms, such as a cyclopropyl group, a cyclobutyl group, a cyclopentyl group, a cyclohexyl group, an adamantyl group, and a norbornyl group. Of these, those having 1 to 6 carbon atoms are preferable.

The hydrocarbylene group represented by R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group represented by R^{a3} or R^{s6} may be saturated or unsaturated, and may be linear, branched, or cyclic. Specific examples thereof include a saturated hydrocarbyl group, and an aliphatic unsaturated hydrocarbyl group such as an alkenyl group and an alkynyl group, and a saturated hydrocarbyl group is preferable. Examples of the saturated hydrocarbyl group include those given as examples of the hydrocarbyl group represented by R^{s1}, and also an undecyl group, a dodecyl group, a tridecyl group, a tetradecyl group, and a pentadecyl group. Examples of the fluorinated hydrocarbyl group represented by R^{s3} or R^{s6} include groups in which some or all of the hydrogen atoms bonded to the carbon atoms of the hydrocarbyl group described above are substituted with fluorine atoms. As described above, an ether bond or a carbonyl group may be present between these carbon-carbon bonds.

Specific examples of the acid-labile group represented by R^{s3} include the groups represented by the above-mentioned general formulae (AL-3) to (AL-5), a trialkylsilyl group in which each alkyl group has 1 to 6 carbon atoms, and an oxo-group-containing alkyl group having 4 to 20 carbon atoms.

The (u+1)-valent hydrocarbon group or fluorinated hydrocarbon group represented by R^{s4} may be linear, branched, or cyclic, and specific examples thereof include groups obtained by further eliminating “u” hydrogen atoms from the above-mentioned hydrocarbyl group or fluorinated hydrocarbyl group.

The fluorinated hydrocarbyl group represented by R^{sa} is preferably saturated, and may be linear, branched, or cyclic. Specific examples thereof include ones in which some or all of the hydrogen atoms of the hydrocarbyl groups have been substituted with fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeating unit represented by any one of the general formulae (7A) to (7E) include, but are not limited to, those shown below. In addition, in the formulae below, RB is as defined above.

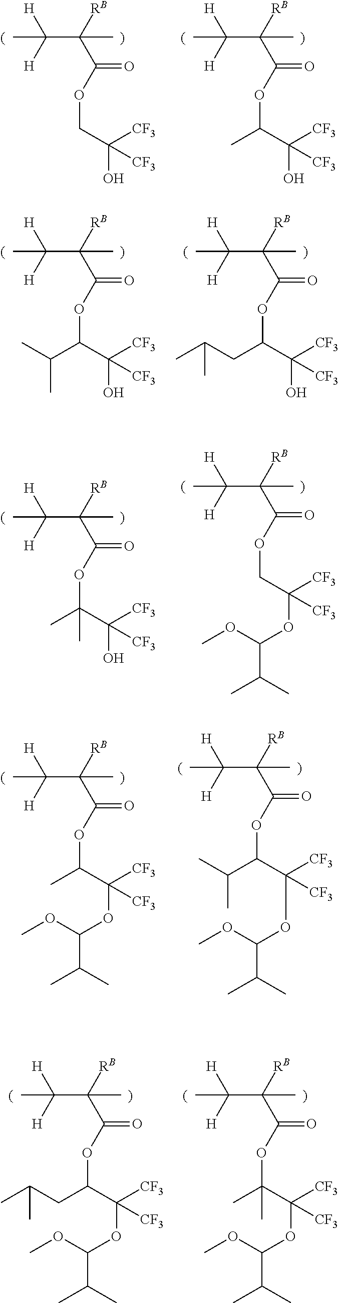

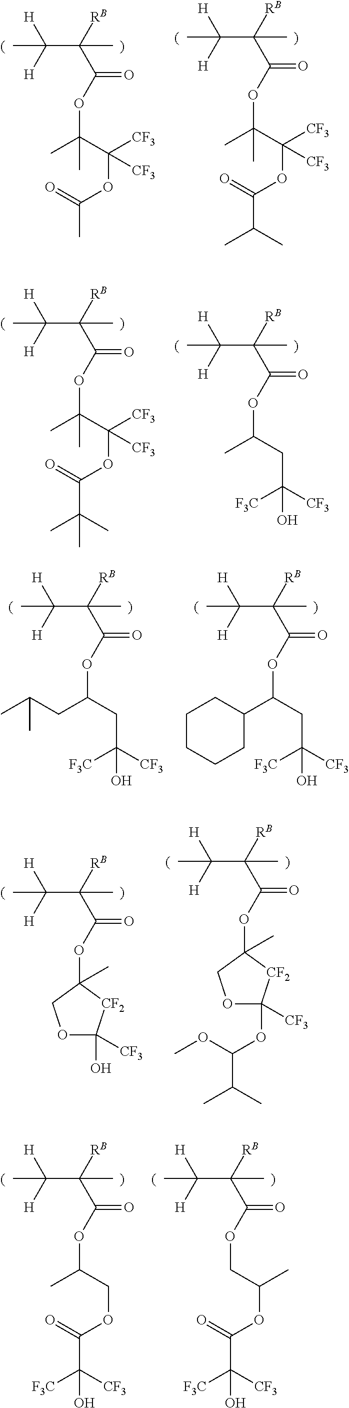

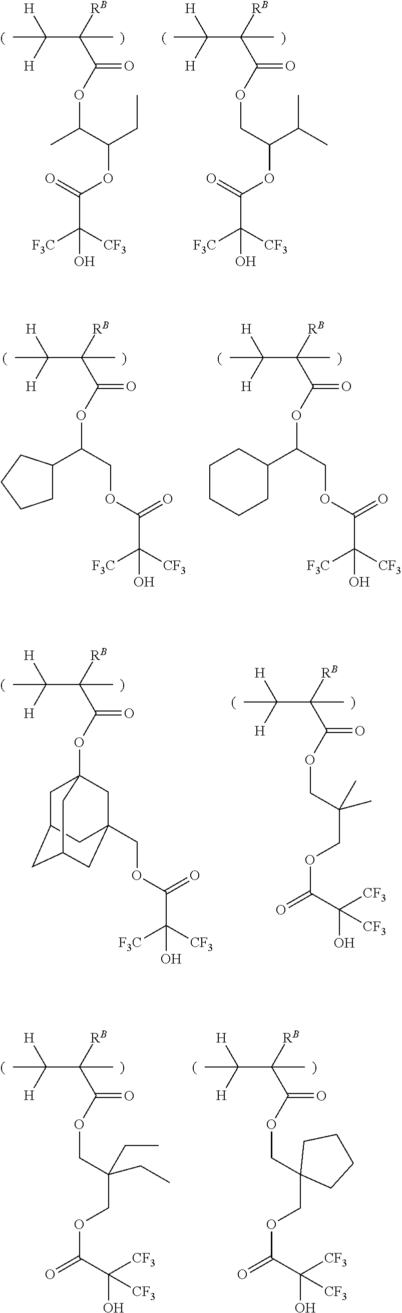

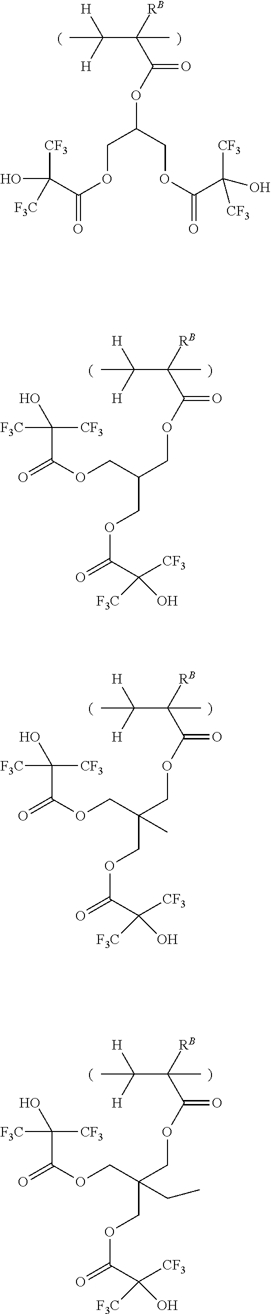

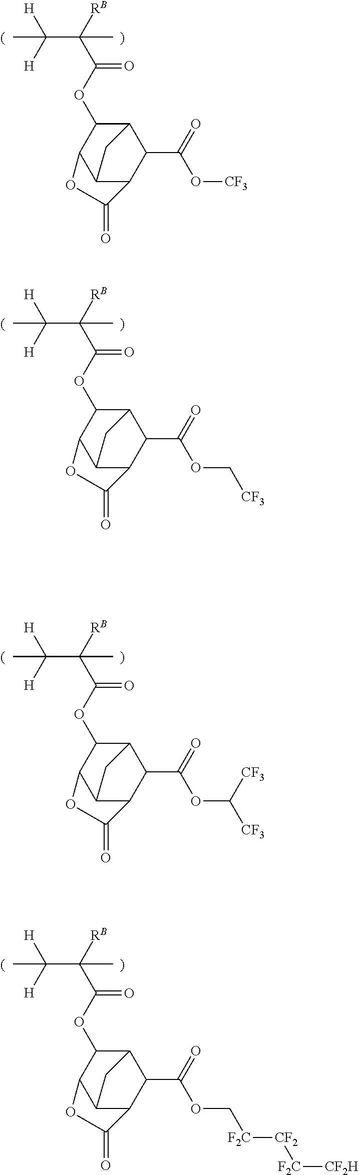

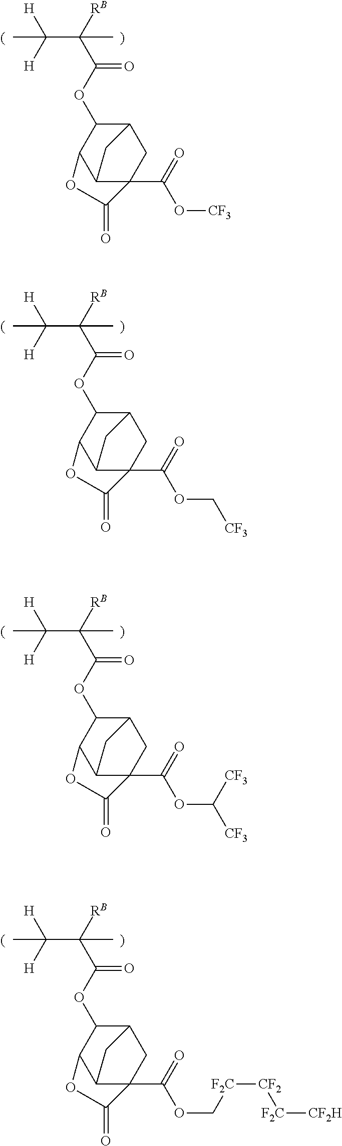

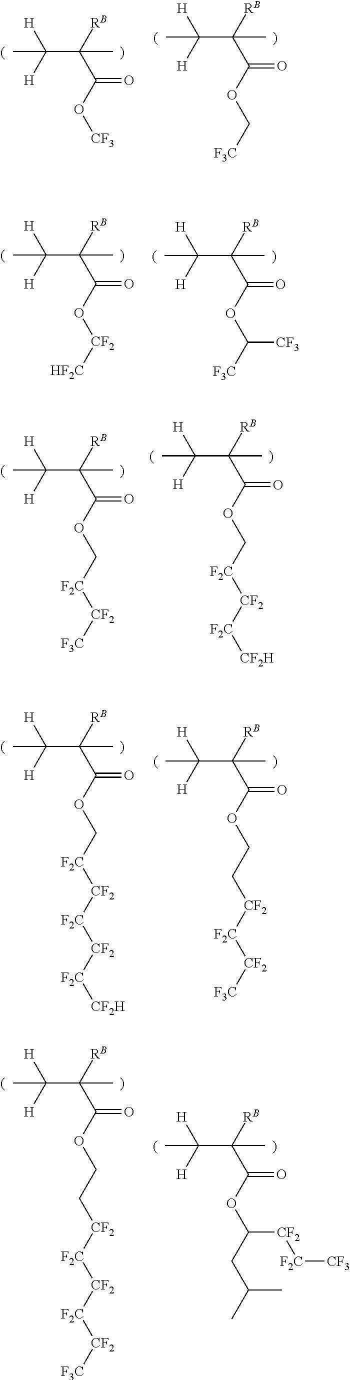

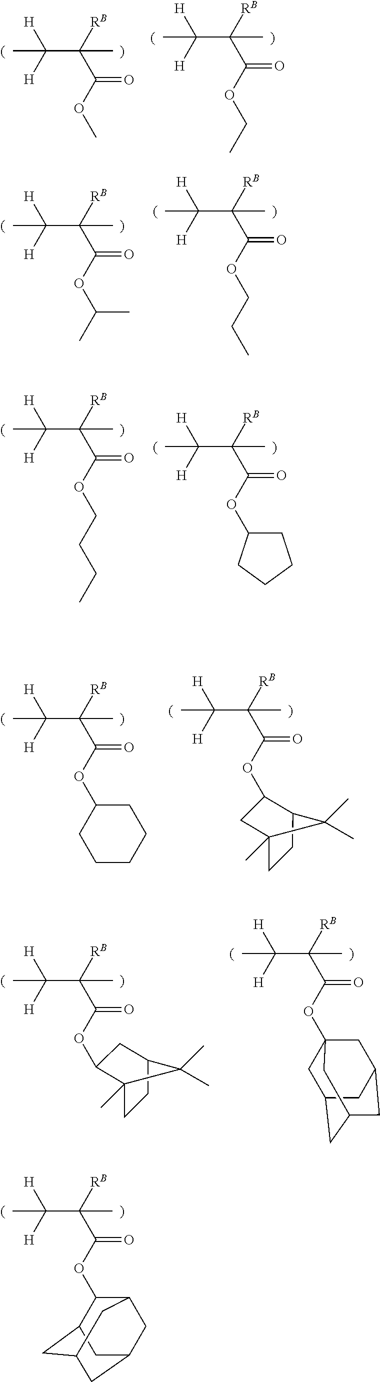

The polymer surfactant may further contain other repeating units in addition to the repeating units represented by the general formulae (7A) to (7E). Examples of other repeating units include repeating units obtained from methacrylic acid and xx-trifluoromethylacrylic acid derivatives. In the polymer surfactant, the content of the repeating units represented by the above general formulae (7A) to (7E) is preferably 20 mol % or more, more preferably 60 mol % or more, and further preferably 100 mol %, of all repeating units.

The Mw of the polymer surfactant is preferably 1,000 to 500,000, and more preferably 3,000 to 100,000. Mw/Mn is preferably 1.0 to 2.0, and more preferably 1.0 to 1.6.

The polymer surfactant can be synthesized by heating a monomer containing an unsaturated bond that gives the repeating units represented by general formulae (7A) to (7E) and other repeating units as necessary in an organic solvent with the addition of a radical initiator to polymerize the monomer. Examples of organic solvents used in polymerization include toluene, benzene, THE, diethyl ether, and dioxane. Examples of polymerization initiators include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid-labile group may be used as is after being introduced into the monomer, or may be protected or partially protected after polymerization.

When synthesizing the polymeric surfactant, known chain transfer agents such as dodecyl mercaptan and 2-mercaptoethanol may be used to adjust the molecular weight. In this case, the amount of these chain transfer agents added is preferably 0.01 to 10 mol % per the total number of moles of the monomers to be polymerized.

When the chemically amplified resist composition of the present invention contains the surfactant (F), the content thereof is preferably 0.1 to 50 parts by mass, and more preferably 0.5 to 10 parts by mass, per 80 parts by mass of the base polymer (B). When the content of the surfactant (F) is 0.1 part by mass or more, the receding contact angle between the resist film surface and water is sufficiently improved, and when the content is 50 parts by mass or less, the dissolution rate of the resist film surface in the developer is low, and the height of the formed fine pattern is sufficiently kept. The surfactant (F) may be used alone or in a combination of two or more types.

### [Other Components (G)]

The chemically amplified resist composition of the present invention may contain, as other components (G), a compound that decomposes with an acid to generate an acid (acid amplifying compound), an organic acid derivative, a fluorine-substituted alcohol, a compound having an Mw of 3,000 or less that changes in solubility in a developer by the action of an acid (dissolution inhibitor), etc. For the acid amplifying compound, the compounds described in JP2009-269953A or JP2010-215608A can be referred to. When the acid amplifying compound is added, the content thereof is preferably 0 to 5 parts by mass, and more preferably 0 to 3 parts by mass, relative to 80 parts by mass of the base polymer (B). When the content falls within this range, suppress acid diffusion can be suppressed, and degradation of resolution and pattern shape is unlikely to occur. For the organic acid derivative, fluorine-substituted alcohols, and dissolution inhibitor, the compounds described in JP2009-269953A or JP2010-215608A can be referred to.

### [Patterning Process]

In addition, in the present invention, a patterning process is provided, wherein the patterning process contains a step of forming a resist film on a substrate using the chemically amplified resist composition described above, a step of exposing the resist film with a high-energy beam, and a step of developing the exposed resist film using a developer.

The substrate may be, for example, a substrate for integrated circuit manufacturing (Si, SiO₂, SiN, SiON, TiN, WSi, BPSG, SOG, organic anti-reflective film, etc.) or a substrate for mask circuit manufacturing (Cr, Cro, CrON, MoSi₂, SiO₂, etc.).

The resist film can be formed, for example, by applying the chemically amplified resist composition onto a substrate by a method such as spin coating so that the film thickness is preferably 0.05 to 2 μm, and then pre-baking the composition on a hot plate, preferably at 60 to 150° C. for 1 to 10 minutes, and more preferably at 80 to 140° C. for 1 to 5 minutes.

Examples of high-energy beams used for exposing the resist film include KrF excimer laser light, ArF excimer laser light, an electron beam (EB), and extreme ultraviolet (EUV) with a wavelength of 3 to 15 nm. When KrF excimer laser light, ArF excimer laser light, or EUV is used, the exposure can be performed by irradiating using a mask for forming a desired pattern so that the exposure amount is preferably 1 to 200 mJ/cm², and more preferably 10 to 100 mJ/cm². When an EB is used, irradiation is performed using a mask for forming a desired pattern or directly so that the exposure amount is preferably 1 to 300 μC/cm², and more preferably 10 to 200 μC/cm².

In addition to the usual exposure method, the immersion method can also be used, in which a liquid with a refractive index of 1.0 or more is placed between the resist film and the projection lens. In this case, a water-insoluble protective film can also be used.

The water-insoluble protective film is used to prevent elution from the resist film and to increase the water-sliding properties of the film surface, and is roughly divided into two types. One is an organic solvent stripping type that needs to be stripped before alkaline aqueous solution development using an organic solvent that does not dissolve the resist film, and the other is an alkaline aqueous solution soluble type that is soluble in an alkaline developer and removes the protective film along with removing the soluble part of the resist film. The latter is based on a polymer having a 1,1,1,3,3,3-hexafluoro-2-propanol residue that is insoluble in water and soluble in an alkaline developer, and is preferably dissolved in an alcohol solvent with 4 or more carbon atoms, an ether solvent with 8 to 12 carbon atoms, or a mixed solvent of these. The above-mentioned water-insoluble and alkaline-develope-soluble surfactant can also be dissolved in an alcohol solvent with 4 or more carbon atoms, an ether solvent with 8 to 12 carbon atoms, or a mixed solvent of these.

After exposure, PEB may be performed. PEB can be performed, for example, by heating on a hot plate, preferably at 60 to 150° C. for 1 to 5 minutes, and more preferably at 80 to 140° C. for 1 to 3 minutes.

For development, for example, an alkaline aqueous developer such as preferably 0.1 to 5% by mass, and more preferably 2 to 3% by mass of tetramethylammonium hydroxide (TMAH) is used, and development is carried out by a conventional method such as a dip method, a puddle method, or a spray method for preferably 0.1 to 3 minutes, and more preferably 0.5 to 2 minutes, until the exposed areas dissolve and the desired pattern is formed on the substrate.

After the resist film is formed, a pure water rinse may be performed to extract the acid generator and other substances from the film surface or to wash away particles, or a rinse may be performed to remove water remaining on the film after exposure.

Furthermore, the pattern may be formed by a double patterning method. Examples of double patterning methods include a trench method in which a 1:3 trench pattern is processed by a first exposure and etching, and then a 1:3 trench pattern is formed by a second exposure with a shifted position to form a 1:1 pattern, and a line method in which a first base with a 1:3 isolated leave pattern is processed by a first exposure and etching, and then a 1:3 isolated leave pattern is formed under the first base with a second exposure with a shifted position to form a 1:1 pattern with half the pitch.

In the patterning process of the present invention, a negative tone development method may be used in which an organic solvent is used as a developer instead of the alkaline aqueous solution to dissolve the unexposed areas.

For the organic solvent development, the developer may be 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, etc. These organic solvents may be used alone or in a combination of two or more types.

### EXAMPLE

The present invention will be specifically described below with reference to Synthesis Examples, Examples, and Comparative Examples. However, the present invention is not limited thereto. The apparatus used is as follows.

### MALDI TOF-MS: JEOL S3000

### [1] Synthesis of Onium Salt

### [Example 1-1] Synthesis of PAG-1

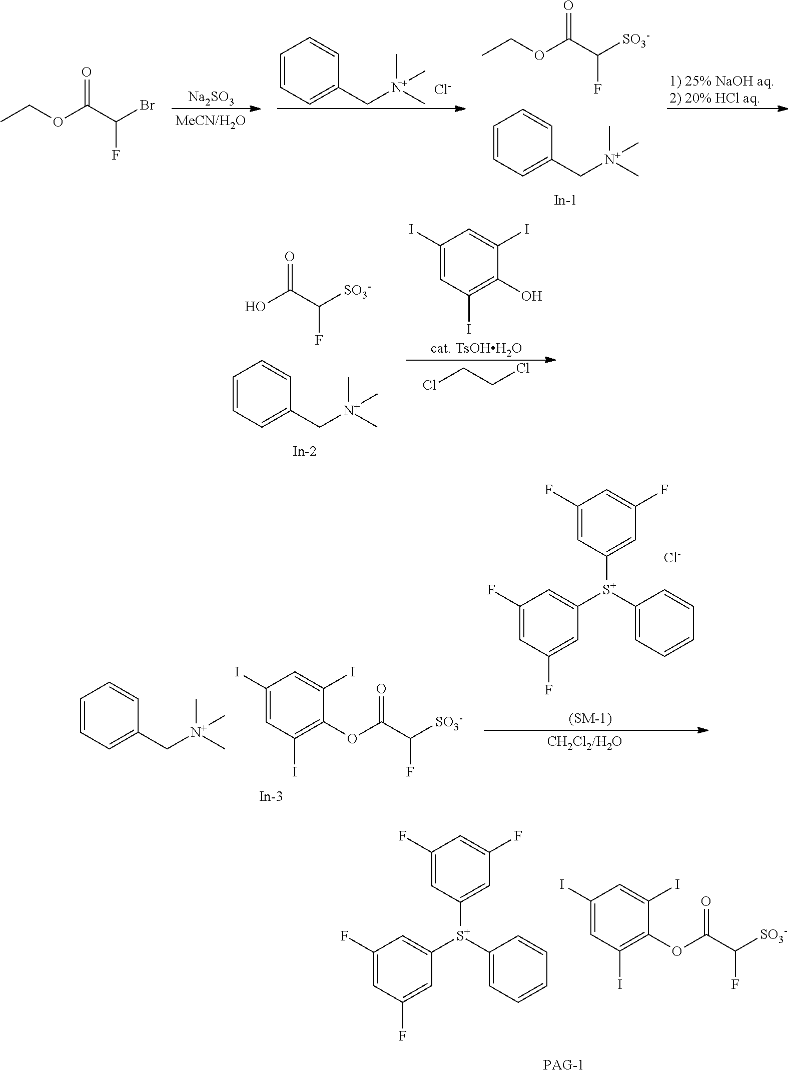

### (1) Synthesis of Intermediate In-1

Under a nitrogen atmosphere, ethyl bromofluoroacetate (30.0 g), sodium sulfite (20.4 g), acetonitrile (120 g), and water (60 g) were added and stirred at 60° C. for 5 hours. The reaction solution was transferred to a separatory funnel, and the aqueous layer was washed twice with hexane (50 g).

Benzyltrimethylammonium chloride (30.1 g) and methylene chloride (150 g) were added to the resulting aqueous solution and stirred at room temperature for 1 hour. The reaction solution was transferred to a separatory funnel, and the organic layer was washed once with water (50 g). After separating the organic layer, the solvent was concentrated to obtain 42.4 g of intermediate In-1 as an oil (yield 78%).

### (2) Synthesis of Intermediate In-2

Under a nitrogen atmosphere, intermediate In-1 (42.4 g) was dissolved in a 25% by mass aqueous solution of sodium hydroxide (22.2 g) and stirred at 40° C. for 6 hours. Then, 20% by mass hydrochloric acid (26.5 g) was added to neutralize the reaction solution. Then, acetonitrile (100 g) was added and azeotropic dehydration was performed to obtain 31.8 g of intermediate In-2 as an oil (yield 82%).

### (3) Synthesis of Intermediate In-3

Under a nitrogen atmosphere, intermediate In-2 (10.1 g), 2,4,6-triiodophenol (14.1 g), p-toluenesulfonic acid monohydrate (0.5 g), and 1,2-dichloroethane (60 g) were mixed and heated to reflux at 90° C. for 6 hours. The reaction mixture was then cooled and water (20 g) was added to stop the reaction. After that, the usual aqueous work-up and solvent distillation were performed, followed by recrystallization with diisopropyl ether, yielding 18.0 g of intermediate In-3 as white crystals (79% yield).

### (4) Synthesis of PAG-1

Under a nitrogen atmosphere, intermediate In-3 (7.6 g), raw material SM-1 (4.5 g), methylene chloride (30 g), and water (15 g) were added and stirred for 30 minutes, and then the organic layer was separated and washed with water, and then concentrated under reduced pressure. Diisopropyl ether was added to the concentrated solution and recrystallized, yielding 8.8 g of the target product, PAG-1, as white crystals (yield 93%).

The TOF-MS results for PAG-1 are shown below.
MALDI TOF-MS: POSITIVE M⁺ 335 (C₁₈H₁₁F₄S⁺-equivalent) NEGATIVE M⁻ 610 (C₈H₃FI₃O₅S⁻-equivalent)

### [Examples 1-2 to 1-10] Synthesis of PAG-2 to PAG-10

Various onium salts were synthesized using the corresponding raw materials and various organic synthesis reactions. The structures of the onium salts used as the photo-acid generator in the chemically amplified resist composition are shown below.

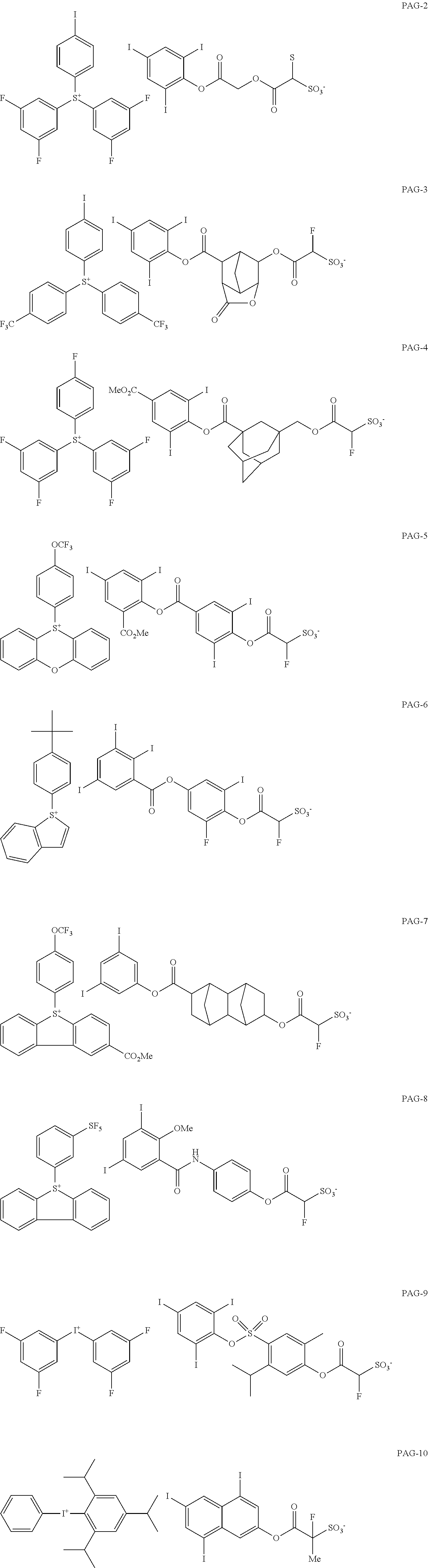

### [Synthesis Examples 1 to 5] Synthesis of Base Polymers (P-1 to P-5)

Each monomer was combined and copolymerized in MEK solvent, the reaction solution was poured into hexane, and the precipitated solid was washed with hexane, isolated, and dried to obtain base polymers (P-1 to P-5) with the compositions shown below. The compositions of the obtained base polymers were confirmed by 1H-NMR, and their Mw and Mw/Mn were confirmed by GPC (solvent: THE, standard: polystyrene).

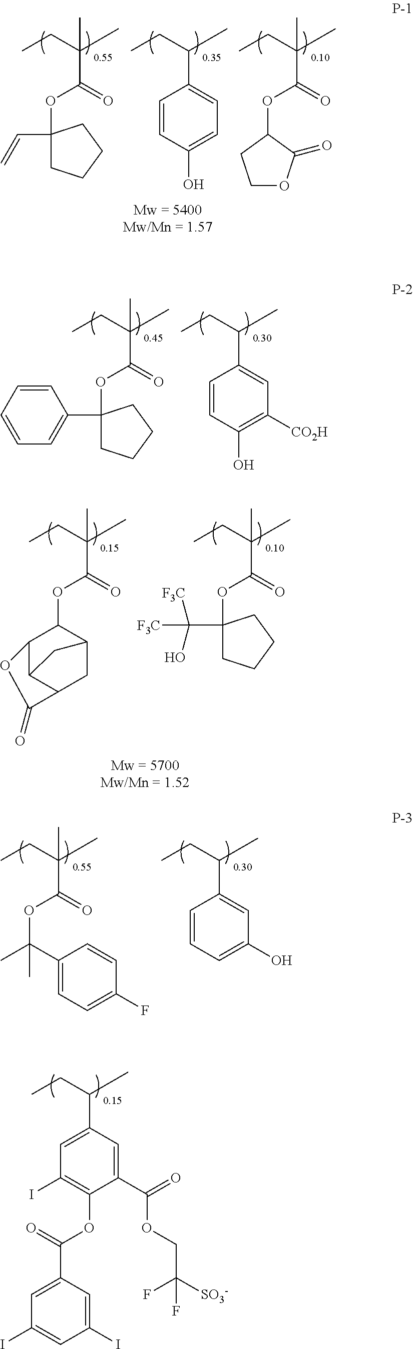

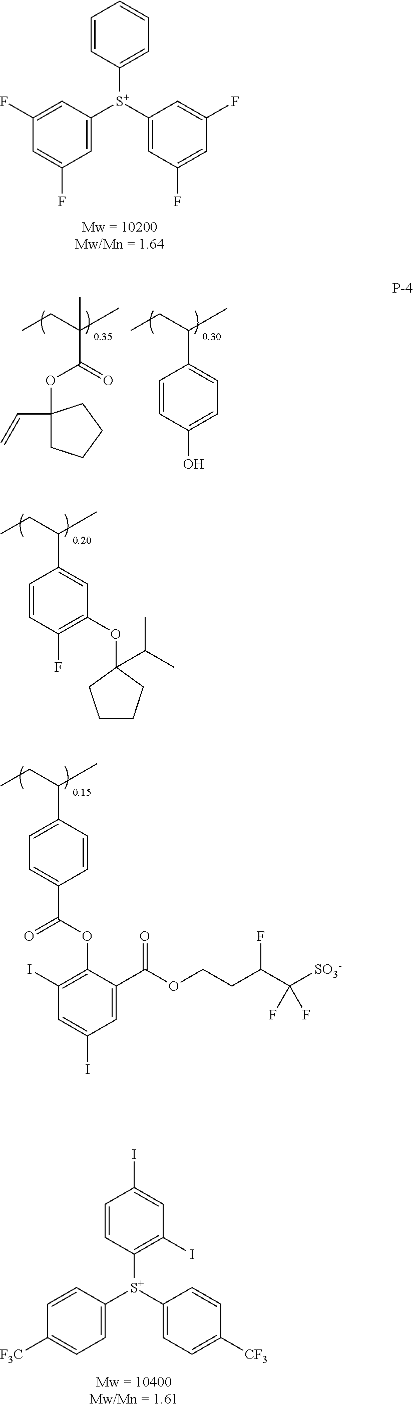

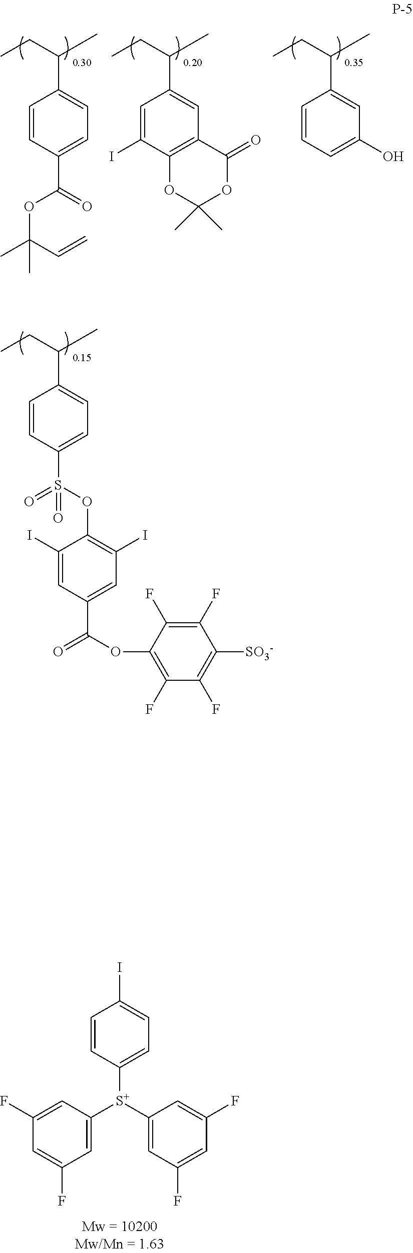

### [3] Preparation of Chemically Amplified Resist Composition

### Examples 2-1 to 2-35, Comparative Examples 1-1 to 1-21

The sulfonium salts of the present invention (PAG-1 to PAG-10), comparative photo-acid generators (PAG-A to PAG-D), other photo-acid generators (PAG-X, PAG-Y), base polymers (P-1 to P-5) and quenchers (Q-1 to Q-4) were dissolved in a solvent containing 0.01 mass % of surfactant A (Omnova) with the compositions shown in Tables 1 and 2 below to prepare solutions, and the solutions were then filtered through a 0.2 μm Teflon (registered trademark) type filter to prepare chemically amplified resist compositions (R-1 to R-35 and CR-1 to CR-21).

TABLE 1

| | | | Photo- | | Other | | | |
|---|---|---|---|---|---|---|---|---|
| | | Base | acid | | Photo-acid | Solvent | Solvent | Solvent |
| | | polymer | generator | Quencher | generator | 1 | 2 | 3 |
| | Resist | (parts | (parts | (parts | (parts | (parts | (parts | (parts |
| | composition | by mass) | by mass) | by mass) | by mass) | by mass) | by mass) | by mass ) |
| Example | R-1 | P-1 | PAG-1 | Q-1 (8.0) | — | PGMEA | EL | DAA |
| 2-1 | | (80) | (24) | | | (2250) | (2800) | (550) |
| Example | R-2 | P-1 | PAG-2 | Q-1 (8.2) | — | PGMEA | EL | DAA |
| 2-2 | | (80) | (24) | | | (2250) | (2800) | (550) |
| Example | R-3 | P-1 | PAG-3 | Q-1 (8.0) | — | PGMEA | EL | DAA |
| 2-3 | | (80) | (24) | | | (2250) | (2800) | (550) |
| Example | R-4 | P-1 | PAG-4 | Q-1 (7.8) | — | PGMEA | EL | DAA |
| 2-4 | | (80) | (24) | | | (2250) | (2800) | (550) |
| Example | R-5 | P-1 | PAG-5 | Q-1 (8.0) | — | PGMEA | EL | DAA |
| 2-5 | | (80) | (24) | | | (2250) | (2800) | (550) |
| Example | R-6 | P-1 | PAG-6 | Q-1 (8.0) | — | PGMEA | EL | DAA |
| 2-6 | | (80) | (24) | | | (2250) | (2800) | (550) |
| Example | R-7 | P-1 | PAG-7 | Q-1 (8.2) | — | PGMEA | EL | DAA |
| 2-7 | | (80) | (24) | | | (2250) | (2800) | (550) |
| Example | R-8 | P-1 | PAG-8 | Q-1 (8.0) | — | PGMEA | EL | DAA |
| 2-8 | | (80) | (24) | | | (2250) | (2800) | (550) |
| Example | R-9 | P-1 | PAG-9 | Q-1 (7.6) | — | PGMEA | EL | DAA |
| 2-9 | | (80) | (24) | | | (2250) | (2800) | (550) |
| Example | R-10 | P-1 | PAG-10 | Q-1 (8.0) | — | PGMEA | EL | DAA |
| 2-10 | | (80) | (24) | | | (2250) | (2800) | (550) |
| Example | R-11 | P-1 | PAG-1 | Q-2 (8.0) | — | PGMEA | EL | DAA |
| 2-11 | | (80) | (24) | | | (2250) | (2800) | (550) |
| Example | R-12 | P-1 | PAG-1 | Q-3 (8.2) | — | PGMEA | EL | DAA |
| 2-12 | | (80) | (24) | | | (2250) | (2800) | (550) |
| Example | R-13 | P-1 | PAG-1 | Q-1 (4.0) | — | PGMEA | EL | DAA |
| 2-13 | | (80) | (24) | Q-4 (3.8) | | (2250) | (2800) | (550) |
| Example | R-14 | P-1 | PAG-1 | Q-3 (8.0) | PAG-X (6) | PGMEA | EL | DAA |
| 2-14 | | (80) | (16) | | | (2250) | (2800) | (550) |
| Example | R-15 | P-1 | PAG-2 | Q-1 (8.0) | PAG-Y (8) | PGMEA | EI | DAA |
| 2-15 | | (80) | (14) | | | (2250) | (2800) | (550) |
| Example | R-16 | P-2 | PAG-1 | Q-1 (8.2) | — | PGMEA | EL | DAA |
| 2-16 | | (80) | (24) | | | (2250) | (2800) | (550) |
| Example | R-17 | P-2 | PAG-2 | Q-1 (8.0) | — | PGMEA | EL | DAA |
| 2-17 | | (80) | (24) | | | (2250) | (2800) | (550) |
| Example | R-18 | P-2 | PAG-4 | Q-3 (7.8) | — | PGMEA | EL | DAA |
| 2-18 | | (80) | (24) | | | (2250) | (2800) | (550) |
| Example | R-19 | P-2 | PAG-6 | Q-2 (8.0) | — | PGMEA | EL | DAA |
| 2-19 | | (80) | (24) | | | (2250) | (2800) | (550) |
| Example | R-20 | P-2 | PAG-9 | Q-1 (8.0) | PAG-Y (12) | PGMEA | EI | DAA |
| 2-20 | | (80) | (12) | | | (2250) | (2800) | (550) |
| Example | R-21 | P-3 | PAG-1 | Q-1 (7.8) | — | PGMEA | EL | DAA |
| 2-21 | | (80) | (24) | | | (2250) | (2800) | (550) |
| Example | R-22 | P-3 | PAG-3 | Q-2 (8.0) | — | PGMEA | EL | DAA |
| 2-22 | | (80) | (24) | | | (2250) | (2800) | (550) |
| Example | R-23 | P-3 | PAG-5 | Q-3 (7.8) | — | PGMEA | EL | DAA |
| 2-23 | | (80) | (24) | | | (2250) | (2800) | (550) |
| Example | R-24 | P-3 | PAG-7 | Q-3 (4.0) | PAG-X (12) | PGMEA | EL | DAA |
| 2-24 | | (80) | (12) | Q-4 (3.8) | | 12250 | (2800) | (550) |
| Example | R-25 | P-3 | PAG-10 | Q-1 (8.2) | — | PGMEA | EL | DAA |
| 2-25 | | (80) | (24) | | | (2250) | (2800) | (550) |
| Example | R-26 | P-4 | PAG-1 | Q-3 (7.8) | — | PGMEA | EL | DAA |
| 2-26 | | (80) | (24) | | | (2250) | (2800) | (550) |
| Example | R-27 | P-4 | PAG-3 | Q-1 (8.0) | — | PGMEA | EL | DAA |
| 2-27 | | (80) | (24) | | | (2250) | (2800) | (550) |
| Example | R-28 | P-4 | PAG-4 | Q-2 (8.2) | — | PGMEA | EL | DAA |
| 2-28 | | (80) | (24) | | | (2250) | (2800) | (550) |
| Example | R-29 | P-4 | PAG-7 | Q-2 (4.0) | — | PGMEA | EL | DAA |
| 2-29 | | (80) | (24) | Q-4 (3.8) | | (2250) | (2800) | (550) |
| Example | R-30 | P-4 | PAG-9 | Q-3 (7.8) | — | PGMEA | EL | DAA |
| 2-30 | | (80) | (24) | | | (2250) | (2800) | (550) |
| Example | R-31 | P-5 | PAG-1 | Q-1 (8.0) | — | PGMEA | EL | DAA |
| 2-31 | | (80) | (24) | | | (2250) | (2800) | (550) |
| Example | R-32 | P-5 | PAG-2 | Q-3 (7.8) | — | PGMEA | EL | DAA |
| 2-32 | | (80) | (24) | | | (2250) | (2800) | (550) |
| Example | R-33 | P-5 | PAG-5 | Q-1 (8.0) | — | PGMEA | EL | DAA |
| 2-33 | | (80) | (24) | | | (2250) | (2800) | (550) |
| Example | R-34 | P-5 | PAG-8 | Q-2 (8.2) | — | PGMEA | EL | DAA |
| 2-34 | | (80) | (24) | | | (2250) | (2800) | (550) |
| Example | R-35 | P-5 | PAG-10 | Q-1 (8.0) | — | PGMEA | EL | DAA |
| 2-35 | | (80) | (24) | | | (2250) | (2800) | (550) |

TABLE 2

| | | | | | Other | | | |
|---|---|---|---|---|---|---|---|---|
| | | | Photo- | | Photo- | | | |
| | | Base | acid | | acid | Solvent | Solvent | Solvent |
| | | polymer | generator | Quencher | generator | 1 | 2 | 3 |
| | Resist | (parts | (parts | (parts | (parts | (parts | (parts | (parts |
| | composition | by mass) | by mass) | by mass) | by mass) | by mass) | by mass) | by mass) |
| Comparative | CR-1 | P-1 | PAG-A | Q-1 (8.0) | — | PGMEA | EL | DAA |
| Example 1-1 | | (80) | (24) | | | (2250) | (2800) | (550) |
| Comparative | CR-2 | P-1 | PAG-B | Q-1 (8.2) | — | PGMEA | EL | DAA |
| Example 1-2 | | (80) | (24) | | | (2250) | (2800) | (550) |
| Comparative | CR-3 | P-1 | PAG-C | Q-1 (8.0) | — | PGMEA | EL | DAA |
| Example 1-3 | | (80) | (24) | | | (2250) | (2800) | (550) |
| Comparative | CR-4 | P-1 | PAG-D | Q-1 (7.8) | — | PGMEA | EL | DAA |
| Example 1-4 | | (80) | (24) | | | (2250) | (2800) | (550) |
| Comparative | CR-5 | P-1 | PAG-A | Q-2 (8.0) | — | PGMEA | EL | DAA |
| Example 1-5 | | (80) | (24) | | | (2250) | (2800) | (550) |
| Comparative | CR-6 | P-1 | PAG-A | Q-3 (8.2) | — | PGMEA | EL | DAA |
| Example 1-6 | | (80) | (24) | | | (2250) | (2800) | (550) |
| Comparative | CR-7 | P-1 | PAG-A | Q-1 (4.0) | — | PGMEA | EL | DAA |
| Example 1-7 | | (80) | (24) | Q-4 (3.8) | | (2250) | (2800) | (550) |
| Comparative | CR-8 | P-1 | PAG-A | Q-3 (8.0) | PAG-X (6) | PGMEA | EL | DAA |
| Example 1-8 | | (80) | (16) | | | (2250) | (2800) | (550) |
| Comparative | CR-9 | P-1 | PAG-B | Q-1 (8.0) | PAG-Y (8) | PGMEA | EL | DAA |
| Example 1-9 | | (80) | (14) | | | (2250) | (2800) | (550) |
| Comparative | CR-10 | P-2 | PAG-A | Q-1 (8.2) | — | PGMEA | EL | DAA |
| Example 1-10 | | (80) | (24) | | | (2250) | (2800) | (550) |
| Comparative | CR-11 | P-2 | PAG-B | Q-2 (8.0) | — | PGMEA | EL | DAA |
| Example 1-11 | | (80) | (24) | | | (2250) | (2800) | (550) |
| Comparative | CR-12 | P-2 | PAG-C | Q-1 (8.0) | PAG-Y (12) | PGMEA | EL | DAA |
| Example 1-12 | | (80) | (12) | | | (2250) | (2800) | (550) |
| Comparative | CR-13 | P-3 | PAG-A | Q-1 (7.8) | — | PGMEA | EL | DAA |
| Example 1-13 | | (80) | (24) | | | (2250) | (2800) | (550) |
| Comparative | CR-14 | P-3 | PAG-C | Q-3 (7.8) | — | PGMEA | EL | DAA |
| Example 1-14 | | (80) | (24) | | | (2250) | (2800) | (550) |
| Comparative | CR-15 | P-3 | PAG-D | Q-3 (4.0) | PAG-X (12) | PGMEA | EL | DAA |
| Example 1-15 | | (80) | (12) | Q-4 (3.8) | | (2250) | (2800) | (550) |
| Comparative | CR-16 | P-4 | PAG-A | Q-3 (7.8) | — | PGMEA | EL | DAA |
| Example 1-16 | | (80) | (24) | | | (2250) | (2800) | (550) |
| Comparative | CR-17 | P-4 | PAG-B | Q-2 (4.0) | — | PGMEA | EL | DAA |
| Example 1-17 | | (80) | (24) | Q-4 (3.8) | | (2250) | (2800) | (550) |
| Comparative | CR-18 | P-4 | PAG-C | Q-3 (7.8) | — | PGMEA | EL | DAA |
| Example 1-18 | | (80) | (24) | | | (2250) | (2800) | (550) |
| Comparative | CR-19 | P-5 | PAG-A | Q-1 (8.0) | — | PGMEA | EL | DAA |
| Example 1-19 | | (80) | (24) | | | (2250) | (2800) | (550) |
| Comparative | CR-20 | P-5 | PAG-C | Q-3 (7.8) | — | PGMEA | EL | DAA |
| Example 1-20 | | (80) | (24) | | | (2250) | (2800) | (550) |
| Comparative | CR-21 | P-5 | PAG-D | Q-1 (8.0) | — | PGMEA | EL | DAA |
| Example 1-21 | | (80) | (24) | | | (2250) | (2800) | (550) |

In Tables 1 and 2, the solvents, other photoacid generators PAG-X and PAG-Y, comparative photo-acid generators PAG-A to PAG-D, quenchers Q-1 to Q-4, and surfactant A are as follows.
Solvent: PGMEA (propylene glycol monomethyl ether acetate)
   EL (Ethyl lactate)
   DAA (Diacetone alcohol)
Other photo-acid generators: PAG-X, PAG-Y

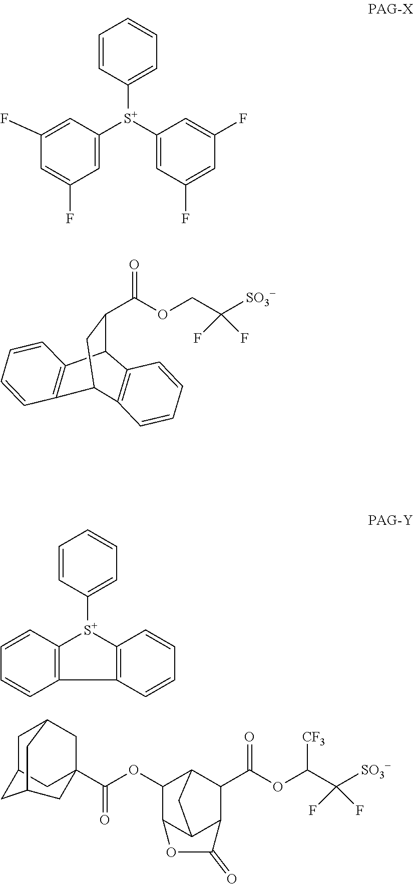

Comparative photo-acid generators: PAG-A to PAG-D

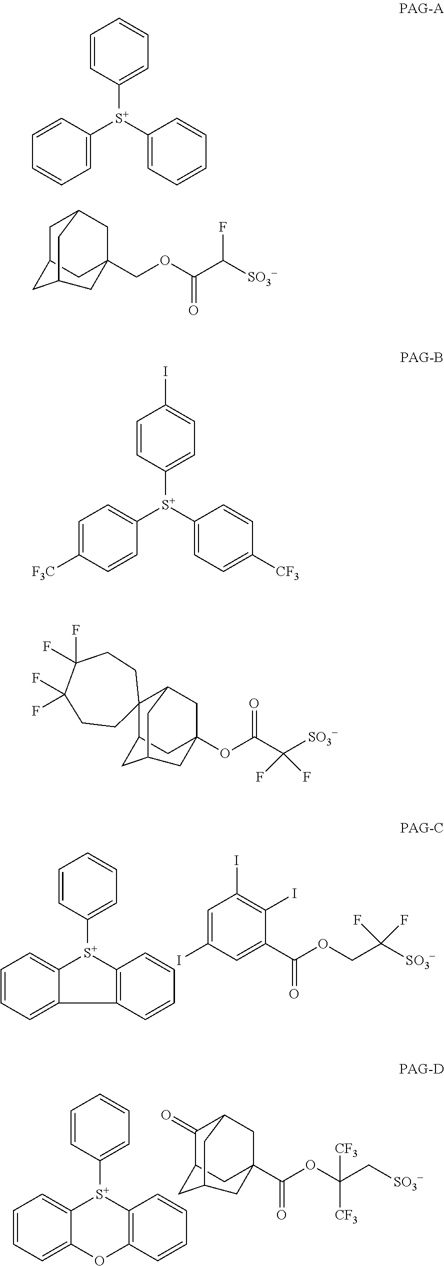

Quenchers: Q-1 to Q-4

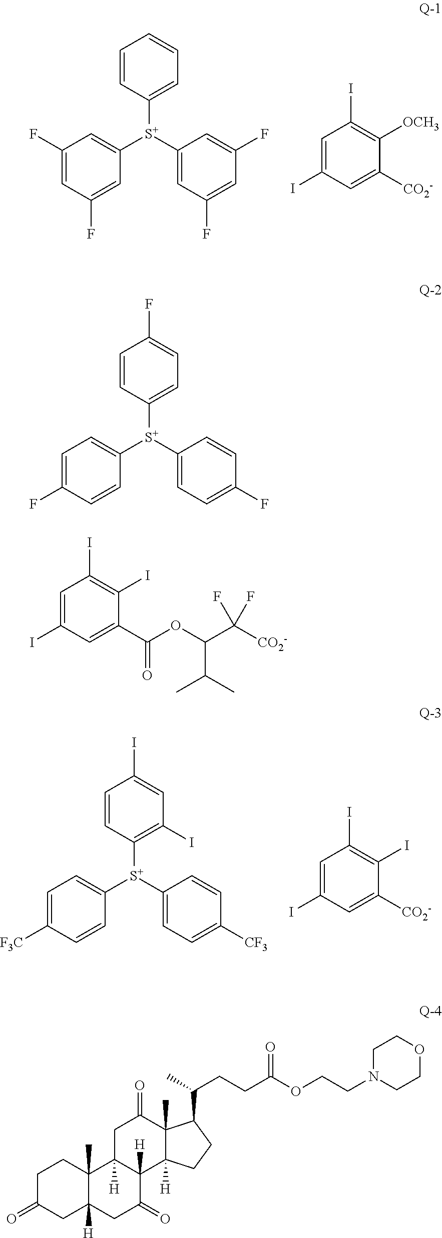

Surfactant A: 3-methyl-3-(2,2,2-trifluoroethoxymethyl) oxetane/tetrahydrofuran/2,2-dimethyl-1,3-propanediol copolymer (manufactured by Omnova)

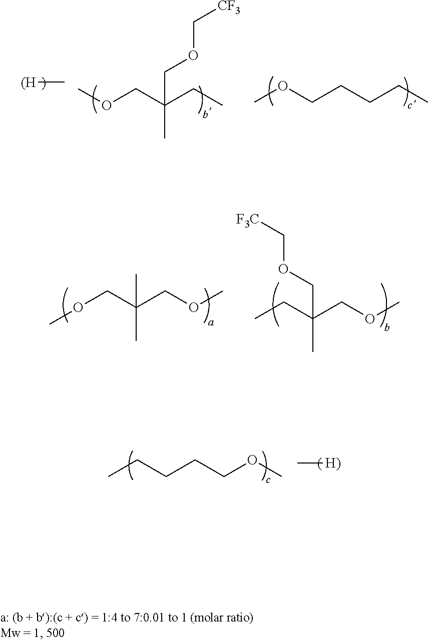

### [4] EUV Lithography Evaluation (1)

### Examples 3-1 to 3-35, Comparative Examples 2-1 to 2-21

Each chemically amplified resist composition (R-1 to R-35, CR-1 to CR-21) shown in Tables 1 and 2 was spin-coated onto an Si substrate on which a silicon-containing spin-on hard mask SHB-A940 (silicon content: 43% by mass) manufactured by Shin-Etsu Chemical Co., Ltd. had been formed to a film thickness of 20 nm, and the substrate was pre-baked at 100° C. for 60 seconds using a hot plate to prepare a resist film with a thickness of 50 nm. The resist film was exposed to an LS pattern with an on-wafer dimension of 18 nm and a pitch of 36 nm using an EUV scanner NXE3400 (NA 0.33, σ 0.9/0.6, dipole illumination) made by ASML, while varying the exposure dose and focus (exposure dose pitch: 1 mJ/cm², focus pitch: 0.020 μm), and after exposure, PEB was performed for 60 seconds at the temperatures shown in Tables 3 and 4. Thereafter, paddle development was carried out with a 2.38% by mass TMAH aqueous solution for 30 seconds, followed by rinsing with a surfactant-containing rinsing material and spin drying to obtain a positive pattern.

The obtained LS pattern was observed with a Hitachi High-Technologies Corporation CD-SEM (CG6300), and the sensitivity, EL, LWR, depth of focus (DOF), and collapse limit were evaluated according to the following methods. The results are shown in Tables 3 and 4.

### [Sensitivity Evaluation]

The optimum exposure dose Eop (mJ/cm²) at which an LS pattern with a line width of 18 nm and a pitch of 36 nm was obtained was used as the sensitivity. The smaller this value, the higher the sensitivity.

### [EL Evaluation]

From the exposure amount formed within a range of ±10% (16.2 to 19.8 nm) of the 18 nm space width in the LS pattern, the EL (unit: %) was calculated by the following formula. The larger this value, the better the performance.

EL(%)=(|E₁−E₂|/Eop)×100
E₁: Optimum exposure dose for providing an LS pattern with a line width of 16.2 nm and a pitch of 36 nm.
E₂: Optimum exposure dose for providing an LS pattern with a line width of 19.8 nm and a pitch of 36 nm.
Eop: Optimum exposure dose for providing an LS pattern with a line width of 18 nm and a pitch of 36 nm.

### [LWR Evaluation]

The LS pattern obtained by irradiation using Eop was measured at 10 points in the longitudinal direction of the lines, then the standard deviation (o) was calculated, and triple its value (30) was used as the LWR. The smaller this value, the smaller the roughness and the more uniform the line width of the pattern obtained.

### [DOF Evaluation]

The depth of focus was evaluated by determining the focus range formed within a range of +10% (16.2 to 19.8 nm) of the 18 nm dimension of the LS pattern. The larger this value, the wider the depth of focus.

### [Collapse Limit Evaluation of Line Pattern]

The line dimension of the LS pattern at each exposure dose at the optimum focus was measured at 10 points in the longitudinal direction. The thinnest line dimension obtained without collapse was used as the collapse limit dimension. The smaller this value, the better the collapse limit.

TABLE 3

| | | PEB | Optimum | | | | Collapse |
|---|---|---|---|---|---|---|---|
| | Resist | temperature | exposure | EL | LWR | DOF | limit |
| | composition | (°C) | (mJ/cm²) | (%) | (nm) | (nm) | (nm) |
| Example 3-1 | R-1 | 95 | 34 | 18 | 2.2 | 110 | 10.6 |
| Example 3-2 | R-2 | 100 | 33 | 19 | 2.3 | 120 | 10.8 |
| Example 3-3 | R-3 | 100 | 34 | 17 | 2.3 | 100 | 10.9 |
| Example 3-4 | R- 4 | 95 | 33 | 18 | 2.3 | 110 | 10.6 |
| Example 3-5 | R-5 | 105 | 32 | 17 | 2.4 | 110 | 11.2 |
| Example 3-6 | R- 6 | 100 | 33 | 18 | 2.2 | 100 | 11.2 |
| Example 3-7 | R-7 | 95 | 34 | 18 | 2.4 | 120 | 10.9 |
| Example 3-8 | R-8 | 95 | 34 | 17 | 2.2 | 110 | 10.9 |
| Example 3-9 | R-9 | 100 | 32 | 16 | 2.3 | 110 | 11.2 |
| Example 3-10 | R-10 | 100 | 33 | 19 | 2.4 | 120 | 11.2 |
| Example 3-11 | R-11 | 100 | 33 | 17 | 2.5 | 110 | 11.0 |
| Example 3-12 | R-12 | 95 | 32 | 18 | 2.3 | 100 | 11.1 |
| Example 3-13 | R-13 | 105 | 34 | 17 | 2.2 | 110 | 10.8 |
| Example 3-14 | R-14 | 100 | 32 | 18 | 2.3 | 120 | 11.1 |
| Example 3-15 | R-15 | 95 | 33 | 18 | 2.3 | 110 | 10.8 |
| Example 3-16 | R-16 | 95 | 33 | 17 | 2.4 | 120 | 10.9 |
| Example 3-17 | R-17 | 100 | 32 | 19 | 2.3 | 120 | 11.3 |
| Example 3-18 | R-18 | 95 | 34 | 16 | 2.3 | 110 | 11.2 |
| Example 3-19 | R-19 | 95 | 32 | 17 | 2.3 | 110 | 11.1 |
| Example 3-20 | R-20 | 100 | 32 | 18 | 2.3 | 120 | 10.8 |
| Example 3-21 | R-21 | 100 | 33 | 17 | 2.4 | 110 | 10.9 |
| Example 3-22 | R-22 | 100 | 33 | 19 | 2.2 | 120 | 11.2 |
| Example 3-23 | R-23 | 95 | 33 | 18 | 2.5 | 100 | 11.2 |
| Example 3-24 | R-24 | 95 | 33 | 18 | 2.3 | 120 | 10.8 |
| Example 3-25 | R-25 | 100 | 33 | 17 | 2.2 | 100 | 11.2 |
| Example 3-26 | R-26 | 100 | 34 | 17 | 2.3 | 110 | 10.9 |
| Example 3-27 | R-27 | 100 | 33 | 18 | 2.4 | 120 | 11.0 |
| Example 3-28 | R-28 | 95 | 33 | 17 | 2.3 | 110 | 11.2 |
| Example 3-29 | R-29 | 95 | 34 | 19 | 2.5 | 100 | 11.1 |
| Example 3-30 | R-30 | 100 | 32 | 18 | 2.5 | 120 | 10.9 |
| Example 3-31 | R-31 | 100 | 34 | 18 | 2.3 | 110 | 10.9 |
| Example 3-32 | R-32 | 100 | 32 | 18 | 2.3 | 110 | 10.8 |
| Example 3-33 | R-33 | 95 | 33 | 17 | 2.4 | 120 | 11.2 |
| Example 3-34 | R-34 | 100 | 32 | 19 | 2.3 | 110 | 10.9 |
| Example 3-35 | R-35 | 100 | 33 | 18 | 2.3 | 110 | 11.3 |

TABLE 4

| | | PEB | Optimum | | | | Collapse |
|---|---|---|---|---|---|---|---|
| | Resist | temperature | exposure | EL | LWR | DOF | limit |
| | composition | (°C) | (mJ/cm²) | (%) | (nm) | (nm) | (nm) |
| Comparative | CR-1 | 95 | 36 | 14 | 2.9 | 100 | 11.9 |
| Example 2-1 | | | | | | | |
| Comparative | CR-2 | 100 | 35 | 13 | 2.8 | 90 | 12.1 |
| Example 2-2 | | | | | | | |
| Comparative | CR-3 | 100 | 35 | 14 | 3.0 | 80 | 12.2 |
| Example 2-3 | | | | | | | |
| Comparative | CR-4 | 100 | 34 | 13 | 3.2 | 80 | 12.1 |
| Example 2-4 | | | | | | | |
| Comparative | CR-5 | 95 | 34 | 15 | 2.8 | 90 | 11.7 |
| Example 2-5 | | | | | | | |
| Comparative | CR-6 | 100 | 35 | 14 | 2.7 | 80 | 11.6 |
| Example 2-6 | | | | | | | |
| Comparative | CR-7 | 100 | 34 | 15 | 2.8 | 80 | 11.6 |
| Example 2-7 | | | | | | | |
| Comparative | CR-8 | 100 | 34 | 14 | 2.8 | 90 | 11.6 |
| Example 2-8 | | | | | | | |
| Comparative | CR-9 | 95 | 35 | 13 | 3.1 | 80 | 12.2 |
| Example 2-9 | | | | | | | |
| Comparative | CR-10 | 100 | 34 | 12 | 2.8 | 70 | 12.1 |
| Example 2-10 | | | | | | | |
| Comparative | CR-11 | 100 | 33 | 15 | 2.7 | 80 | 11.7 |
| Example 2-11 | | | | | | | |
| Comparative | CR-12 | 100 | 34 | 14 | 2.8 | 90 | 11.5 |
| Example 2-12 | | | | | | | |
| Comparative | CR-13 | 95 | 33 | 13 | 2.7 | 80 | 11.6 |
| Example 2-13 | | | | | | | |
| Comparative | CR-14 | 105 | 34 | 14 | 3.1 | 70 | 11.8 |
| Example 2-14 | | | | | | | |
| Comparative | CR-15 | 100 | 35 | 15 | 2.7 | 90 | 11.6 |
| Example 2-15 | | | | | | | |
| Comparative | CR-16 | 95 | 34 | 14 | 2.8 | 90 | 11.5 |
| Example 2-16 | | | | | | | |
| Comparative | CR-17 | 95 | 34 | 13 | 2.8 | 80 | 12.1 |
| Example 2-17 | | | | | | | |
| Comparative | CR-18 | 100 | 34 | 14 | 2.7 | 100 | 12.0 |
| Example 2-18 | | | | | | | |
| Comparative | CR-19 | 95 | 35 | 14 | 2.8 | 90 | 11.7 |
| Example 2-19 | | | | | | | |
| Comparative | CR-20 | 95 | 35 | 13 | 3.0 | 80 | 11.6 |
| Example 2-20 | | | | | | | |
| Comparative | CR-21 | 100 | 34 | 14 | 2.8 | 70 | 11.7 |
| Example 2-21 | | | | | | | |

The results shown in Tables 3 and 4 indicate that the chemically amplified resist composition containing a photo-acid generator consisting of an onium salt of the present invention has good sensitivity and is excellent in terms of EL, LWR, and DOF. In addition, it was also confirmed that the collapse limit value was small and that the composition was resistant to pattern collapse even in fine pattern formation. Therefore, it was demonstrated that the chemically amplified resist composition of the present invention is suitable as a material for EUV lithography.

### [5] EUV Lithography Evaluation (2)

### Examples 4-1 to 4-35, Comparative Examples 3-1 to 3-21

Each chemically amplified resist composition (R-1 to R-35, CR-1 to CR-21) shown in Tables 1 and 2 was spin-coated onto an Si substrate on which a silicon-containing spin-on hard mask SHB-A940 (silicon content: 43% by mass) manufactured by Shin-Etsu Chemical Co., Ltd. had been formed to a film thickness of 20 nm, and the substrate was pre-baked at 105° C. for 60 seconds using a hot plate to produce a resist film with a film thickness of 50 nm. The resist film was exposed using an ASML EUV scanner NXE3400 (NA 0.33, σ 0.9/0.6, quadruple pole illumination, a hole pattern mask with a pitch of 46 nm and a +20% bias on the wafer), PEB was performed for 60 seconds at the temperatures listed in Tables 5 and 6 using a hot plate, and development was performed for 30 seconds with a 2.38% by mass TMAH aqueous solution to form a hole pattern with a dimension of 23 nm.

Using a Hitachi High-Technologies Corporation length-measuring SEM (CG6300), the exposure dose when a hole dimension of 23 nm was formed was measured and used as the sensitivity, and the dimensions of 50 holes were also measured, and the standard deviation (o) calculated from the results was tripled (30) and used as the CDU. The results are shown in Tables 5 and 6.

TABLE 5

| | | PEB | Optimum | |
|---|---|---|---|---|
| | Resist | temperature | exposure | CDU |
| | composition | (°C) | (mJ/cm²) | (nm) |
| Example 4-1 | R-1 | 90 | 23 | 2.2 |
| Example 4-2 | R-2 | 95 | 23 | 2.3 |
| Example 4-3 | R-3 | 90 | 24 | 2.4 |
| Example 4-4 | R-4 | 90 | 24 | 2.3 |
| Example 4-5 | R-5 | 90 | 23 | 2.2 |
| Example 4-6 | R-6 | 95 | 25 | 2.5 |
| Example 4-7 | R-7 | 95 | 24 | 2.3 |
| Example 4-8 | R-8 | 90 | 23 | 2.2 |
| Example 4-9 | R-9 | 95 | 23 | 2.2 |
| Example 4-10 | R-10 | 95 | 24 | 2.3 |
| Example 4-11 | R-11 | 95 | 22 | 2.4 |
| Example 4-12 | R-12 | 90 | 24 | 2.5 |
| Example 4-13 | R-13 | 90 | 25 | 2.4 |
| Example 4-14 | R-14 | 90 | 24 | 2.4 |
| Example 4-15 | R-15 | 90 | 24 | 2.4 |
| Example 4-16 | R-16 | 85 | 24 | 2.3 |
| Example 4-17 | R-17 | 95 | 23 | 2.2 |
| Example 4-18 | R-18 | 95 | 23 | 2.4 |
| Example 4-19 | R-19 | 90 | 24 | 2.5 |
| Example 4-20 | R-20 | 95 | 24 | 2.4 |
| Example 4-21 | R-21 | 95 | 23 | 2.2 |
| Example 4-22 | R-22 | 95 | 22 | 2.4 |
| Example 4-23 | R-23 | 95 | 25 | 2.3 |
| Example 4-24 | R-24 | 90 | 24 | 2.3 |
| Example 4-25 | R-25 | 95 | 23 | 2.4 |
| Example 4-26 | R-26 | 95 | 22 | 2.3 |
| Example 4-27 | R-27 | 95 | 23 | 2.3 |
| Example 4-28 | R-28 | 95 | 24 | 2.4 |
| Example 4-29 | R-29 | 90 | 23 | 2.5 |
| Example 4-30 | R-30 | 95 | 22 | 2.3 |
| Example 4-31 | R-31 | 95 | 25 | 2.4 |
| Example 4-32 | R-32 | 90 | 24 | 2.4 |
| Example 4-33 | R-33 | 95 | 23 | 2.3 |
| Example 4-34 | R-34 | 90 | 23 | 2.4 |
| Example 4-35 | R-35 | 90 | 24 | 2.2 |

TABLE 6

| | | PEB | Optimum | |
|---|---|---|---|---|
| | Resist | temperature | exposure | CDU |
| | composition | (°C) | (mJ/cm²) | (nm) |
| Comparative | CR-1 | 90 | 28 | 3.0 |
| Example 3-1 | | | | |
| Comparative | CR-2 | 95 | 26 | 2.9 |
| Example 3-2 | | | | |
| Comparative | CR-3 | 95 | 26 | 2.7 |
| Example 3-3 | | | | |
| Comparative | CR-4 | 90 | 25 | 2.7 |
| Example 3-4 | | | | |
| Comparative | CR-5 | 90 | 26 | 2.8 |
| Example 3-5 | | | | |
| Comparative | CR-6 | 95 | 27 | 2.7 |
| Example 3-6 | | | | |
| Comparative | CR-7 | 90 | 26 | 2.8 |
| Example 3-7 | | | | |
| Comparative | CR-8 | 90 | 26 | 2.7 |
| Example 3-8 | | | | |
| Comparative | CR-9 | 90 | 27 | 2.8 |
| Example 3-9 | | | | |
| Comparative | CR-10 | 95 | 26 | 2.7 |
| Example 3-10 | | | | |
| Comparative | CR-11 | 95 | 26 | 2.8 |
| Example 3-11 | | | | |
| Comparative | CR-12 | 95 | 25 | 2.8 |
| Example 3-12 | | | | |
| Comparative | CR-13 | 90 | 27 | 3.0 |
| Example 3-13 | | | | |
| Comparative | CR-14 | 95 | 27 | 2.8 |
| Example 3-14 | | | | |
| Comparative | CR-15 | 95 | 27 | 2.7 |
| Example 3-15 | | | | |
| Comparative | CR-16 | 95 | 27 | 2.7 |
| Example 3-16 | | | | |
| Comparative | CR-17 | 95 | 28 | 2.8 |
| Example 3-17 | | | | |
| Comparative | CR-18 | 95 | 26 | 2.8 |
| Example 3-18 | | | | |
| Comparative | CR-19 | 90 | 27 | 2.7 |
| Example 3-19 | | | | |
| Comparative | CR-20 | 95 | 25 | 2.8 |
| Example 3-20 | | | | |
| Comparative | CR-21 | 95 | 26 | 2.7 |
| Example 3-21 | | | | |

The results shown in Tables 5 and 6 confirm that the chemically amplified resist composition containing a photo-acid generator consisting of an onium salt of the present invention has good sensitivity and excellent CDU.

The present description includes the following embodiments.
[1]: An onium salt, wherein the onium salt is represented by the following general formula (1),

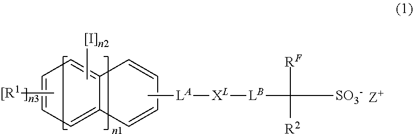

wherein “n1” is 0 or 1; “n2” is an integer from 1 to 4; “n3” is an integer from 0 to 3; provided that when “n1” is 0, 1≤n2+n3≤4, and when “n1” is 1, 1≤n2+n3≤6; R¹ is a halogen atom other than an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” is 2 or 3, each R¹ may be the same as or different from each other, and a plurality of R¹ may be bonded to each other to form a ring together with the carbon atoms to which they are bonded; R² is a hydrogen atom, or a hydrocarbyl group having 1 to 20 carbon atoms and optionally containing a heteroatom; L^{A} and L^{B} are each independently a single bond, an ether bond, an ester bond, a sulfonate ester bond, an amide bond, a sulfonamide bond, a carbonate bond, or a carbamate bond; X^{L} is a single bond, or a hydrocarbylene group having 1 to 40 carbon atoms and optionally containing a heteroatom; RE is a fluorine atom, or a fluorinated saturated hydrocarbyl group having 1 to 6 carbon atoms; and Z⁺ represents an onium
[2]: The onium salt according to [1], wherein the onium salt is represented by the following general formula (1A),

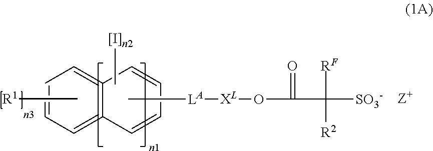

wherein “n1” to “n3”, R¹, R², R^{F}, L^{A}, X^{L}, and Z⁺ are the same as defined above.
[3]: The onium salt according to [2], wherein the onium salt represented by the general formula (1A) is represented by the following general formula (1B),

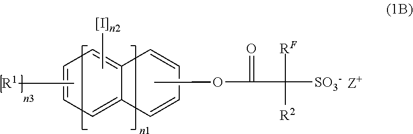

wherein “n1” to “n3”, R¹, R², R^{F}, and Z⁺ are as defined above.
[4]: The onium salt according to any one of [1] to [3], wherein Z⁺ is a sulfonium cation represented by the following general formula (Z-1) or an iodonium cation represented by the following general formula (Z-2),

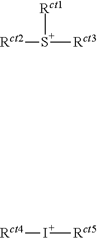

wherein R^{ct1} to R^{ct5} are each independently a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.
[5]: The onium salt according to any one of [1] to [3], wherein Z⁺ is a sulfonium cation represented by the following general formula (Z-3),

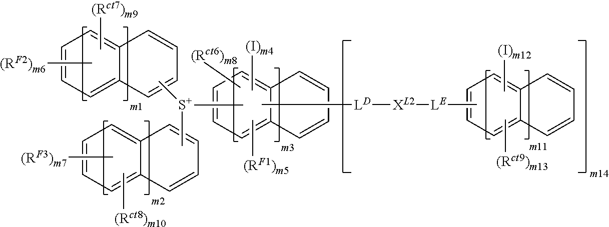

wherein “m1” is 0 or 1; “m2” is 0 or 1; “m3” is 0 or 1; “m4” is an integer from 0 to 4; “m5” is an integer from 0 to 4; “m6” is an integer from 0 to 6; “m7” is an integer from 0 to 6; “m8” is 0, 1 or 2; “m9” is 0, 1 or 2; “m10” is 0, 1 or 2; “m11” is 0 or 1; “m12” is an integer from 0 to 4; “m13” is 0, 1 or 2; “m14” is 0, 1 or 2; provided that when “m1” is 0, 0≤m6+m9≤4, and when “m1” is 1, 0≤m6+m9≤6; when “m2” is 0, 0≤m7+m1054, and when “m2” is 1, 0≤m7+m10≤6; when “m3” is 0, 1≤m4+m5+m8+m14≤4, and when “m3” is 1, 1≤m4+m5+m8+m14≤6; when “m11” is 0, 0≤m12+m13≤4, and when “m11” is 1, 0≤m12+m13≤6; also, 1≤m4+m12;
R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms; when “m5” is 2 or more, each REL may be the same as different from each other, when “m6” is 2 or more, each R^{F2} may be the same as or different from each other, and when “m7” is 2 or more, each R^{F3} may be the same as or different from each other;
R^{ct6} to R^{ct9} are a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “m8” is 2, two R^{ct6} may be the same as or different from each other, and two R^{ct6} may be bonded to each other to form a ring together with the carbon atoms to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two R^{ct9} may be bonded to each other to form a ring together with the carbon atoms to which they are bonded;
also, the aromatic rings directly bonded to S⁺ in the sulfonium cation may be bonded to each other to form a ring together with S⁺;
L^{D} and L^{E} are each independently a single bond, an ether bond, an ester bond, an amide bond, a sulfonate ester bond, a sulfonamide bond, a carbonate bond, or a carbamate bond; and
X¹² is a single bond, or a hydrocarbylene group having 1 to 40 carbon atoms and optionally containing a heteroatom.
[6]: A photo-acid generator, wherein the photo-acid generator comprises the onium salt according to any one of [1] to [5].
[7]: A chemically amplified resist composition, wherein the chemically amplified resist composition comprises the photo-acid generator according to [6].
[8]: The chemically amplified resist composition according to [7], wherein the chemically amplified resist composition further comprises a base polymer containing at least one repeating unit selected from the group consisting of a repeating unit represented by the following general formula (a1), a repeating unit represented by the following general formula (a2), and a repeating unit represented by the following general formula (a3),

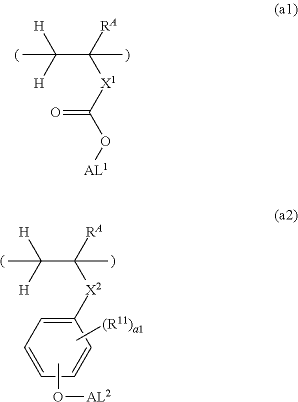

wherein “a1” is an integer from 0 to 4;
R^{A} is each independently a hydrogen atom, a fluorine atom, a methyl group, or a trifluoromethyl group;
X¹ is a single bond, a phenylene group, a naphthylene group, *—C(═O)—OX¹¹—, or *—C(═O)—NH—X¹¹, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ is a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² is a single bond, *—C(═O)—O—, or *—C(═O)—NH—;
“*” represents a bond to a main chain carbon atom;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2, 3, or 4, each R¹¹ may be the same as or different from each other; and
AL¹ and AL² are each independently an acid-labile group,

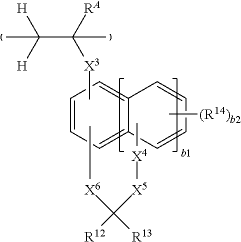

wherein “b1” is 0 or 1; “b2” is an integer from 0 to 3 when “b1” is 0, and an integer from 0 to 5 when “b1” is 1;
R^{A} is a hydrogen atom, a fluorine atom, a methyl group, or a trifluoromethyl group;
X³ is a single bond, *—C(═O)—O—, or *—C(═O)—NH—; “*” represents a bond to a main chain carbon atom;
X⁴ is a single bond, an aliphatic hydrocarbylene group having 1 to 4 carbon atoms, a carbonyl group, a sulfonyl group, or a group obtained by combining these;
X⁵ and X⁶ are each independently an oxygen atom or a sulfur atom; provided that X⁴ and X⁶ are bonded to adjacent carbon atoms of an aromatic ring;
R¹² and R¹³ are each independently a hydrogen atom or a hydrocarbyl group having 1 to 20 carbon atoms and optionally containing a heteroatom; also, R¹² and R¹³ may be bonded to each other to form a ring together with the carbon atom to which they are bonded;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, or a hydrocarbylthio group having 1 to 20 carbon atoms and optionally containing a heteroatom, or —N(R^{14A})(R^{14B}); R^{14A} and R^{14B} are each independently a hydrogen atom or a hydrocarbyl group having 1 to 6 carbon atoms; and when “b2” is 2 or more, each R¹⁴ may be the same as or different from each other, and a plurality of R¹⁴ may be bonded to each other to form a ring together with the carbon atoms of the aromatic ring to which they are bonded.
[9]: The chemically amplified resist composition according to [8], wherein the base polymer further comprises at least one repeating unit selected from the group consisting of a repeating unit represented by the following general formula (b1) and a repeating unit represented by the following general formula (b2),

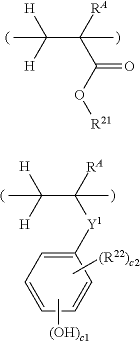

wherein “c1” is an integer from 1 to 4; “c2” is an integer from 0 to 4; and 1≤c1+c2≤5;
R^{A} is each independently a hydrogen atom, a fluorine atom, a methyl group, or a trifluoromethyl group;
Y¹ is a single bond or *—C(═O)—O—; “*” represents a bond to a main chain carbon atom;
R²¹ is a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R²² is a halogen atom, a hydroxy group, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” is 2, 3, or 4, each R²² may be the same as or different from each other.
[10]: The chemically amplified resist composition according to [8] or [9], wherein the base polymer further comprises at least one selected from a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5),

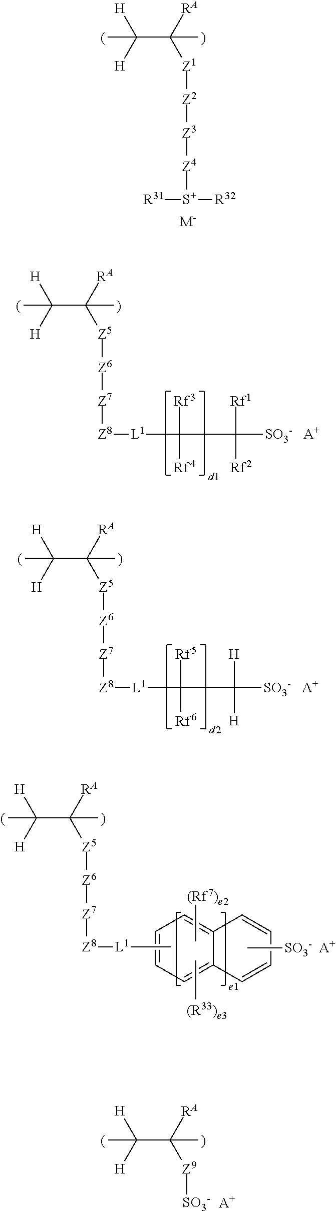

wherein “dl” and “d2” each independently represent an integer from 0 to 3; “e1” is 0 or 1; “e2” is an integer from 0 to 4; “e3” is an integer from 0 to 4; provided that when “e1” is 0, 0≤e2+e354, and when “e1” is 1, 0≤e2+e356;
R^{A} is each independently a hydrogen atom, a fluorine atom, a methyl group, or a trifluoromethyl group;
Z¹ is a single bond, or a phenylene group optionally containing a substituent;
Z² is a single bond, **—C(═O)—O—Z²¹—, **—C(═O)—NH—Z²¹—, or **—O—Z²¹—; Z²¹ is an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ is a single bond, an ether bond, an ester bond, an amide bond, a sulfonate ester bond, a sulfonamide bond, a carbonate bond, or a carbamate bond;
Z⁴ is a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ is each independently a single bond, a phenylene group or a naphthylene group optionally containing a substituent, or *—C(═O)—O—Z⁵¹—; Z⁵¹ is an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ is a single bond, an ether bond, an ester bond, an amide bond, a sulfonate ester bond, a sulfonamide bond, a carbonate bond, or a carbamate bond;
Z⁷ is each independently a single bond, ***—271-C(═O)—O—, ***—C(═O)—NH—Z⁷¹—, or ***—O—Z⁷¹—; Z⁷¹ is a hydrocarbylene group having 1 to 20 carbon atoms and optionally containing a heteroatom;
Z⁸ is each independently a single bond, ****—781-C(═O)—O—, ****—C(═O)—NH—Z⁸¹— or ****—O—Z⁸¹—; Z⁸¹ is a hydrocarbylene group having 1 to 20 carbon atoms and optionally containing a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹— or *—O—Z⁹¹—; Z⁹¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
“*” represents a bond to a main chain carbon atom; “**” represents a bond to Z¹; “***” represents a bond to Z⁶; “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom, or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms; provided that all Rf⁵ and Rf⁶ are not simultaneously hydrogen atoms;
Rf⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R³¹ and R³² are each independently a hydrocarbyl group having 1 to 20 carbon atoms and optionally containing a heteroatom; also, R³¹ and R³² may be bonded to each other to form a ring together with the sulfur atom to which they are bonded;
R³³ is a halogen atom other than a fluorine atom, or hydrocarbyl group having 1 to 20 carbon atoms and optionally containing a heteroatom; when “e3” is 2, 3, or 4, each R³³ may be the same as or different from each other, and a plurality of R³³ may be bonded to each other to form a ring together with the carbon atoms to which they are bonded;
M⁻ is a non-nucleophilic counter ion; and
A⁺ is an onium cation.
[11]: The chemically amplified resist composition according to any one of [7] to [10], wherein the chemically amplified resist composition further comprises at least one selected from the group consisting of an organic solvent, a quencher, a photo-acid generator other than the photo-acid generator, and a surfactant.
[12]: A patterning process, wherein the pattering process comprises a step of forming a resist film on a substrate using the chemically amplified resist composition according to any one of [7] to [11], a step of exposing the resist film with a high-energy beam, and a step of developing the exposed resist film using a developer.
[13]: The patterning method according to [12], wherein the high-energy beam is KrF excimer laser light, ArF excimer laser light, an electron beam, or extreme ultraviolet having a wavelength of 3 to 15 nm.

It should be noted that the present invention is not limited to the embodiments described above. The embodiments described above are just examples, and any examples that have substantially the same features and demonstrate the same functions and effects as those in the technical concepts disclosed in the claims of the present invention are included in the technical scope of the present invention.

1. An onium salt, wherein the onium salt is represented by the following general formula (1), 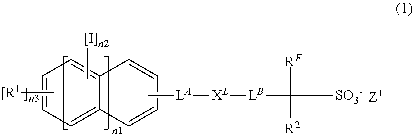
wherein “n1” is 0 or 1; “n2” is an integer from 1 to 4; “n3” is an integer from 0 to 3; provided that when “n1” is 0, 1≤n2+n3≤4, and when “n1” is 1, 1≤n2+n3≤6; R¹ is a halogen atom other than an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “n3” is 2 or 3, each R¹ may be the same as or different from each other, and a plurality of R¹ may be bonded to each other to form a ring together with the carbon atoms to which they are bonded; R² is a hydrogen atom, or a hydrocarbyl group having 1 to 20 carbon atoms and optionally containing a heteroatom; L^{A} and L^{B} are each independently a single bond, an ether bond, an ester bond, a sulfonate ester bond, an amide bond, a sulfonamide bond, a carbonate bond, or a carbamate bond; X^{L} is a single bond, or a hydrocarbylene group having 1 to 40 carbon atoms and optionally containing a heteroatom; R^{F} is a fluorine atom, or a fluorinated saturated hydrocarbyl group having 1 to 6 carbon atoms; and Z⁺ represents an onium cation.

2. The onium salt according to claim 1, wherein the onium salt is represented by the following general formula (1A), 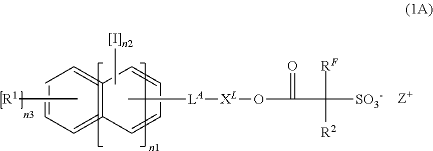
wherein “n1” to “n3”, R¹, R², R^{F}, L^{A}, X^{L}, and Z⁺ are the same as defined above.

3. The onium salt according to claim 2, wherein the onium salt represented by the general formula (1A) is represented by the following general formula (1B), 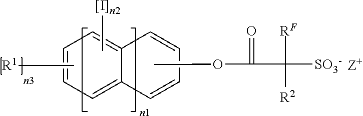
wherein “n1” to “n3”, R¹, R², R^{F}, and Z⁺ are as defined above.

4. The onium salt according to claim 1, wherein Z⁺ is a sulfonium cation represented by the following general formula (Z-1) or an iodonium cation represented by the following general formula (Z-2), 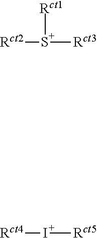
wherein R^{ct1} to R^{ct5} are each independently a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

5. The onium salt according to claim 1, wherein Z⁺ is a sulfonium cation represented by the following general formula (Z-3), 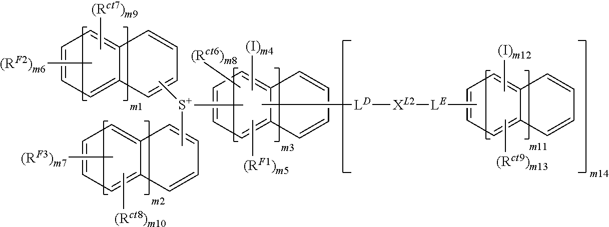
wherein “m1” is 0 or 1; “m2” is 0 or 1; “m3” is 0 or 1; “m4” is an integer from 0 to 4; “m5” is an integer from 0 to 4; “m6” is an integer from 0 to 6; “m7” is an integer from 0 to 6; “m8” is 0, 1 or 2; “m9” is 0, 1 or 2; “m10” is 0, 1 or 2; “m11” is 0 or 1; “m12” is an integer from 0 to 4; “m13” is 0, 1 or 2; “m14” is 0, 1 or 2; provided that when “m1” is 0, 0≤m6+m9≤4, and when “m1” is 1, 0≤m6+m9≤6; when “m2” is 0, 0≤m7+m10≤4, and when “m2” is 1, 0≤m7+m10≤6; when “m3” is 0, 1≤m4+m5+m8+m14≤4, and when “m3” is 1, 1≤m4+m5+m8+m14≤6; when “m11” is 0, 0≤m12+m13≤4, and when “m11” is 1, 0≤m12+m13≤6; also, 1≤m4+m12;
R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms; when “m5” is 2 or more, each REL may be the same as different from each other, when “m6” is 2 or more, each R^{F2} may be the same as or different from each other, and when “m7” is 2 or more, each R^{F3} may be the same as or different from each other;
R^{ct6} to R^{ct9} are a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; when “m8” is 2, two R^{ct6} may be the same as or different from each other, and two R^{ct6} may be bonded to each other to form a ring together with the carbon atoms to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two R^{ct9} may be bonded to each other to form a ring together with the carbon atoms to which they are bonded;
also, the aromatic rings directly bonded to S⁺ in the sulfonium cation may be bonded to each other to form a ring together with S⁺;
L^{D} and L^{E} are each independently a single bond, an ether bond, an ester bond, an amide bond, a sulfonate ester bond, a sulfonamide bond, a carbonate bond, or a carbamate bond; and
X^{L2} is a single bond, or a hydrocarbylene group having 1 to 40 carbon atoms and optionally containing a heteroatom.

6. A photo-acid generator, wherein the photo-acid generator comprises the onium salt according to claim 1.

7. A chemically amplified resist composition, wherein the chemically amplified resist composition comprises the photo-acid generator according to claim 6.

8. The chemically amplified resist composition according to claim 7, wherein the chemically amplified resist composition further comprises a base polymer containing at least one repeating unit selected from the group consisting of a repeating unit represented by the following general formula (a1), a repeating unit represented by the following general formula (a2), and a repeating unit represented by the following general formula (a3), 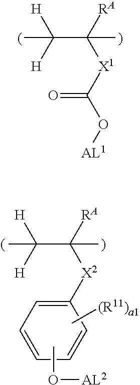
wherein “a1” is an integer from 0 to 4;
R^{A} is each independently a hydrogen atom, a fluorine atom, a methyl group, or a trifluoromethyl group;
X¹ is a single bond, a phenylene group, a naphthylene group, *—C(═O)—OX¹¹—, or *—C(═O)—NH—X¹¹, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ is a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² is a single bond, *—C(═O)—O—, or *—C(═O)—NH—;
“*” represents a bond to a main chain carbon atom;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2, 3, or 4, each R¹¹ may be the same as or different from each other; and
A^{L1} and A^{L2} are each independently an acid-labile group, 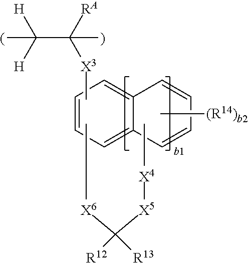
wherein “b1” is 0 or 1; “b2” is an integer from 0 to 3 when “b1” is 0, and an integer from 0 to 5 when “b1” is 1;
R^{A} is a hydrogen atom, a fluorine atom, a methyl group, or a trifluoromethyl group;
X³ is a single bond, *—C(═O)—O—, or *—C(═O)—NH—; “*” represents a bond to a main chain carbon atom;
X⁴ is a single bond, an aliphatic hydrocarbylene group having 1 to 4 carbon atoms, a carbonyl group, a sulfonyl group, or a group obtained by combining these;
X⁵ and X⁶ are each independently an oxygen atom or a sulfur atom; provided that X⁴ and X⁶ are bonded to adjacent carbon atoms of an aromatic ring;
R¹² and R¹³ are each independently a hydrogen atom or a hydrocarbyl group having 1 to 20 carbon atoms and optionally containing a heteroatom; also, R¹² and R¹³ may be bonded to each other to form a ring together with the carbon atom to which they are bonded;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, or a hydrocarbylthio group having 1 to 20 carbon atoms and optionally containing a heteroatom, or —N(R^{14A})(R^{14B}); R^{14A} and R^{14B} are each independently a hydrogen atom or a hydrocarbyl group having 1 to 6 carbon atoms; and when “b2” is 2 or more, each R¹⁴ may be the same as or different from each other, and a plurality of R¹⁴ may be bonded to each other to form a ring together with the carbon atoms of the aromatic ring to which they are bonded.

9. The chemically amplified resist composition according to claim 8, wherein the base polymer further comprises at least one repeating unit selected from the group consisting of a repeating unit represented by the following general formula (b1) and a repeating unit represented by the following general formula (b2), 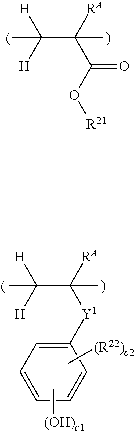
wherein “c1” is an integer from 1 to 4; “c2” is an integer from 0 to 4; and 1≤c1+c2≤5;
R^{A} is each independently a hydrogen atom, a fluorine atom, a methyl group, or a trifluoromethyl group;
Y¹ is a single bond or *—C(═O)—O—; “*” represents a bond to a main chain carbon atom;
R²¹ is a hydrogen atom, or a group having 1 to 20 carbon atoms containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R²² is a halogen atom, a hydroxy group, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” is 2, 3, or 4, each R²² may be the same as or different from each other.

10. The chemically amplified resist composition according to claim 8, wherein the base polymer further comprises at least one selected from a repeating unit represented by the following general formula (c1), a repeating unit represented by the following general formula (c2), a repeating unit represented by the following general formula (c3), a repeating unit represented by the following general formula (c4), and a repeating unit represented by the following general formula (c5), 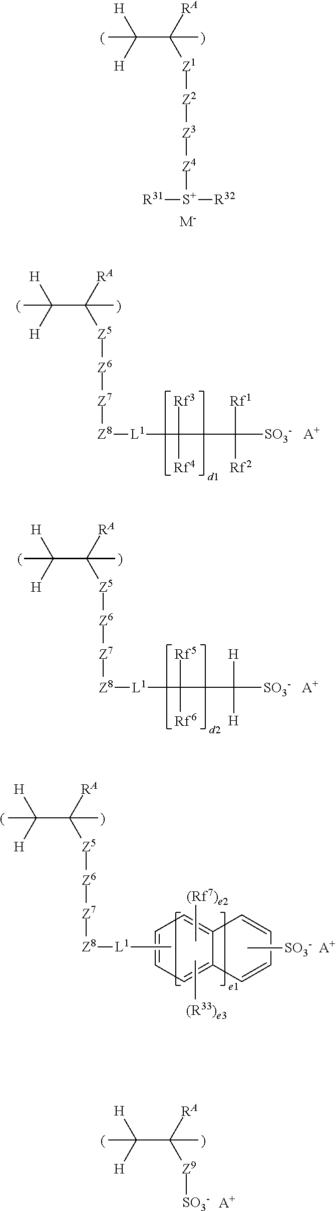
wherein “d1” and “d2” each independently represent an integer from 0 to 3; “e1” is 0 or 1; “e2” is an integer from 0 to 4; “e3” is an integer from 0 to 4; provided that when “e1” is 0, 0≤e2+e354, and when “e1” is 1, 0≤e2+e356;
R^{A} is each independently a hydrogen atom, a fluorine atom, a methyl group, or a trifluoromethyl group;
Z¹ is a single bond, or a phenylene group optionally containing a substituent;
Z² is a single bond, **—C(═O)—O—Z²¹—, **—C(═O)—NH—Z²¹—, or **—O—Z²¹—; Z²¹ is an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z³ is a single bond, an ether bond, an ester bond, an amide bond, a sulfonate ester bond, a sulfonamide bond, a carbonate bond, or a carbamate bond;
Z⁴ is a single bond, or an aliphatic hydrocarbylene group having 1 to 6 carbon atoms, a phenylene group, or a divalent group obtained by combining these, and may contain a halogen atom, a carbonyl group, an ester bond, an ether bond, or a hydroxy group;
Z⁵ is each independently a single bond, a phenylene group or a naphthylene group optionally containing a substituent, or *—C(═O)—O—Z⁵¹—; Z⁵¹ is an aliphatic hydrocarbylene group having 1 to 10 carbon atoms, a phenylene group, or a naphthylene group, and the aliphatic hydrocarbylene group may contain a halogen atom, a hydroxy group, an ether bond, an ester bond, or a lactone ring;
Z⁶ is a single bond, an ether bond, an ester bond, an amide bond, a sulfonate ester bond, a sulfonamide bond, a carbonate bond, or a carbamate bond;
Z⁷ is each independently a single bond, ***—Z⁷¹—C(═O)—O—, ***—C(═O)—NH—Z⁷¹—, or ***—O—Z⁷¹—; Z⁷¹ is a hydrocarbylene group having 1 to 20 carbon atoms and optionally containing a heteroatom;
Z⁸ is each independently a single bond, ****—Z⁸¹—C(═O)—O—, ****—C(═O)—NH—Z⁸¹— or ****—O—Z⁸¹—; Z⁸¹ is a hydrocarbylene group having 1 to 20 carbon atoms and optionally containing a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹— or *—O—Z⁹¹—; Z⁹¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond, or a hydroxy group;
“*” represents a bond to a main chain carbon atom; “**” represents a bond to Z¹; “***” represents a bond to Z⁶; “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom, or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms; provided that all Rf⁵ and Rf⁶ are not simultaneously hydrogen atoms;
Rf⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R³¹ and R³² are each independently a hydrocarbyl group having 1 to 20 carbon atoms and optionally containing a heteroatom; also, R³¹ and R³² may be bonded to each other to form a ring together with the sulfur atom to which they are bonded;
R³³ is a halogen atom other than a fluorine atom, or hydrocarbyl group having 1 to 20 carbon atoms and optionally containing a heteroatom; when “e3” is 2, 3, or 4, each R³³ may be the same as or different from each other, and a plurality of R³³ may be bonded to each other to form a ring together with the carbon atoms to which they are bonded;
M⁻ is a non-nucleophilic counter ion; and
A⁺ is an onium cation.

11. The chemically amplified resist composition according to claim 7, wherein the chemically amplified resist composition further comprises at least one selected from the group consisting of an organic solvent, a quencher, a photo-acid generator other than the photo-acid generator, and a surfactant.

12. A patterning process, wherein the pattering process comprises a step of forming a resist film on a substrate using the chemically amplified resist composition according to claim 7, a step of exposing the resist film with a high-energy beam, and a step of developing the exposed resist film using a developer.

13. The patterning method according to claim 12, wherein the high-energy beam is KrF excimer laser light, ArF excimer laser light, an electron beam, or extreme ultraviolet having a wavelength of 3 to 15 nm.
